SEQUENCE LISTING (40 sequences)

SEQ ID NO: 1 - RNA, 60 nt, synthetic construct
  source 1..60: mol_type = other RNA
  modified_base 1: mod_base = OTHER; note = 5' Fluorescein modified nucleotide
agggaagggctgggatggcagtagacttggctttcccattactcttttctaaaaaaaaaa

SEQ ID NO: 2 - RNA, 50 nt, synthetic construct
  source 1..50: mol_type = other RNA
  modified_base 1: mod_base = OTHER; note = 5' Fluorescein modified nucleotide
agggaagggctgggatggcagtagacttggctttcccattactcttttct

SEQ ID NO: 3 - DNA, 14 bp, synthetic construct
  source 1..14: mol_type = other DNA
  modified_base 1: mod_base = OTHER; note = Nucleotide with phosphorothioate linkage
  modified_base 2: mod_base = OTHER; note = Locked nucleic acid with phosphorothioate linkage
  modified_base 3: mod_base = OTHER; note = Nucleotide with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Locked nucleic acid with phosphorothioate linkage
  modified_base 7: mod_base = OTHER; note = Nucleotide with phosphorothioate linkage
  modified_base 8: mod_base = OTHER; note = Locked nucleic acid with phosphorothioate linkage
  modified_base 9: mod_base = OTHER; note = Nucleotide with phosphorothioate linkage
  modified_base 10: mod_base = OTHER; note = Locked nucleic acid with phosphorothioate linkage
  modified_base 11: mod_base = OTHER; note = Nucleotide with phosphorothioate linkage
  modified_base 12: mod_base = OTHER; note = Locked nucleic acid with phosphorothioate linkage
  modified_base 13: mod_base = OTHER; note = Nucleotide with phosphorothioate linkage
  modified_base 14: mod_base = OTHER; note = Locked nucleic acid
gaagtacacattgg

SEQ ID NO: 4 - DNA, 15 bp, synthetic construct
  source 1..15: mol_type = other DNA
  modified_base 1: mod_base = OTHER; note = Nucleotide with phosphorothioate linkage
  modified_base 2: mod_base = OTHER; note = Locked nucleic acid with phosphorothioate linkage
  modified_base 3: mod_base = OTHER; note = Nucleotide with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Locked nucleic acid with phosphorothioate linkage
  modified_base 7: mod_base = OTHER; note = Nucleotide with phosphorothioate linkage
  modified_base 8: mod_base = OTHER; note = Locked nucleic acid with phosphorothioate linkage
  modified_base 9: mod_base = OTHER; note = Nucleotide with phosphorothioate linkage
  modified_base 10: mod_base = OTHER; note = Locked nucleic acid with phosphorothioate linkage
  modified_base 11: mod_base = OTHER; note = Nucleotide with phosphorothioate linkage
  modified_base 12: mod_base = OTHER; note = Locked nucleic acid with phosphorothioate linkage
  modified_base 13: mod_base = OTHER; note = Nucleotide with phosphorothioate linkage
  modified_base 14: mod_base = OTHER; note = Locked nucleic acid with phosphorothioate linkage
gactgtaggtttatc

SEQ ID NO: 5 - DNA, 20 bp, synthetic construct
  source 1..20: mol_type = other DNA
tggcctgtgttgcttatgaa

SEQ ID NO: 6 - DNA, 18 bp, synthetic construct
  source 1..18: mol_type = other DNA
ccaatgtgatctggaact

SEQ ID NO: 7 - DNA, 18 bp, synthetic construct
  source 1..18: mol_type = other DNA
ccaatgtgatctggaact

SEQ ID NO: 8 - DNA, 20 bp, synthetic construct
  source 1..20: mol_type = other DNA
cgtaccaggtagcgagaaag

SEQ ID NO: 9 - DNA, 20 bp, synthetic construct
  source 1..20: mol_type = other DNA
tgatctttgcagtgacccag

SEQ ID NO: 10 - DNA, 16 bp, synthetic construct
  source 1..16: mol_type = other DNA
cttggcgtgtgaagat

SEQ ID NO: 11 - DNA, 16 bp, synthetic construct
  source 1..16: mol_type = other DNA
cttggcgtgtgaagat

SEQ ID NO: 12 - DNA, 20 bp, synthetic construct
  source 1..20: mol_type = other DNA
ctagagcatctccagcacac

SEQ ID NO: 13 - DNA, 24 bp, synthetic construct
  source 1..24: mol_type = other DNA
gaaactcttgggacctggtgtgaa

SEQ ID NO: 14 - DNA, 20 bp, synthetic construct
  source 1..20: mol_type = other DNA
ttattcagggtgtcagatca

SEQ ID NO: 15 - DNA, 25 bp, synthetic construct
  source 1..25: mol_type = other DNA
cttgccagccatgttttctttgcaa

SEQ ID NO: 16 - DNA, 105 bp, synthetic construct
  source 1..105: mol_type = other DNA
  misc_difference 1..105: note = This sequence may encompass 6-35 cag repeating units
cagcagcagcagcagcagcagcagcagcagcagcagcagcagcagcagcagcagcagcagcagcagcagcagcagcagcagcagcagcagcagcagcagcagcag

SEQ ID NO: 17 - DNA, 108 bp, synthetic construct
  source 1..108: mol_type = other DNA
  misc_difference 1..108: note = This sequence may encompass 9-36 cag repeating units
cagcagcagcagcagcagcagcagcagcagcagcagcagcagcagcagcagcagcagcagcagcagcagcagcagcagcagcagcagcagcagcagcagcagcagcag

SEQ ID NO: 18 - DNA, 96 bp, synthetic construct
  source 1..96: mol_type = other DNA
  misc_difference 1..96: note = This sequence may encompass 14-32 cag repeating units
cagcagcagcagcagcagcagcagcagcagcagcagcagcagcagcagcagcagcagcagcagcagcagcagcagcagcagcagcagcagcagcag

SEQ ID NO: 19 - DNA, 120 bp, synthetic construct
  source 1..120: mol_type = other DNA
  misc_difference 1..120: note = This sequence may encompass 12-40 cag repeating units
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 20 - DNA, 54 bp, synthetic construct
  source 1..54: mol_type = other DNA
  misc_difference 1..54: note = This sequence may encompass 4-18 cag repeating units
cagcagcagcagcagcagcagcagcagcagcagcagcagcagcagcagcagcag

SEQ ID NO: 21 - DNA, 51 bp, synthetic construct
  source 1..51: mol_type = other DNA
  misc_difference 1..51: note = This sequence may encompass 7-17 cag repeating units
cagcagcagcagcagcagcagcagcagcagcagcagcagcagcagcagcag

SEQ ID NO: 22 - DNA, 126 bp, synthetic construct
  source 1..126: mol_type = other DNA
  misc_difference 1..126: note = This sequence may encompass 25-42 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 23 - DNA, 750 bp, synthetic construct
  source 1..750: mol_type = other DNA
  misc_difference 1..750: note = This sequence may encompass 36-250 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 24 - DNA, 264 bp, synthetic construct
  source 1..264: mol_type = other DNA
  misc_difference 1..264: note = This sequence may encompass 49-88 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 25 - DNA, 186 bp, synthetic construct
  source 1..186: mol_type = other DNA
  misc_difference 1..186: note = This sequence may encompass 38-62 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 26 - DNA, 231 bp, synthetic construct
  source 1..231: mol_type = other DNA
  misc_difference 1..231: note = This sequence may encompass 33-77 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 27 - DNA, 258 bp, synthetic construct
  source 1..258: mol_type = other DNA
  misc_difference 1..258: note = This sequence may encompass 55-86 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 28 - DNA, 90 bp, synthetic construct
  source 1..90: mol_type = other DNA
  misc_difference 1..90: note = This sequence may encompass 21-30 cag repeating units
cagcagcagcagcagcagcagcagcagcagcagcagcagcagcagcagcagcagcagcagcagcagcagcagcagcagcagcagcagcag

SEQ ID NO: 29 - DNA, 360 bp, synthetic construct
  source 1..360: mol_type = other DNA
  misc_difference 1..360: note = This sequence may encompass 38-120 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 30 - DNA, 189 bp, synthetic construct
  source 1..189: mol_type = other DNA
  misc_difference 1..189: note = This sequence may encompass 47-63 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 31 - DNA, 159 bp, synthetic construct
  source 1..159: mol_type = other DNA
  misc_difference 1..159: note = This sequence may encompass 6-53 cgg repeating units
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 32 - DNA, 111 bp, synthetic construct
  source 1..111: mol_type = other DNA
  misc_difference 1..111: note = This sequence may encompass 5-37 ctg repeating units
ctgctgctgctgctgctgctgctgctgctgctgctgctgctgctgctgctgctgctgctgctgctgctgctgctgctgctgctgctgctgctgctgctgctgctgctgctg

SEQ ID NO: 33 - DNA, 102 bp, synthetic construct
  source 1..102: mol_type = other DNA
  misc_difference 1..102: note = This sequence may encompass 7-34 gaa repeating units
gaagaagaagaagaagaagaagaagaagaagaagaagaagaagaagaagaagaagaagaagaagaagaagaagaagaagaagaagaagaagaagaagaagaa

SEQ ID NO: 34 - DNA, 111 bp, synthetic construct; identical to SEQ ID NO: 32
  source 1..111: mol_type = other DNA
  misc_difference 1..111: note = This sequence may encompass 16-37 ctg repeating units

SEQ ID NO: 35 - DNA, 160 bp, synthetic construct
  source 1..160: mol_type = other DNA
  misc_difference 1..160: note = This sequence may encompass 9-32 attct repeating units
attctattctattctattctattctattctattctattctattctattctattctattctattctattctattctattctattctattctattctattctattctattctattctattctattctattctattctattctattctattctattctattct

SEQ ID NO: 36 - DNA, 84 bp, synthetic construct
  source 1..84: mol_type = other DNA
  misc_difference 1..84: note = This sequence may encompass 7-28 cag repeating units
cagcagcagcagcagcagcagcagcagcagcagcagcagcagcagcagcagcagcagcagcagcagcagcagcagcagcagcag

SEQ ID NO: 37 - DNA, 750 bp, synthetic construct
  source 1..750: mol_type = other DNA
  misc_difference 1..750: note = This sequence may encompass 110-250 ctg repeating units
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 38 - DNA, 22500 bp, synthetic construct
  source 1..22500: mol_type = other DNA
  misc_difference 1..22500: note = This sequence may encompass 800-4500 attct repeating units
attctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattct

SEQ ID NO: 39 - DNA, 234 bp, synthetic construct
  source 1..234: mol_type = other DNA
  misc_difference 1..234: note = This sequence may encompass 66-78 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 40 - RNA, 10 nt, synthetic construct
  source 1..10: mol_type = other RNA
aaaaaaaaaa

(19) United States
(12) Patent Application Publication
(10) Pub. No.: US 2026/0199485 A1
(43) Pub. Date: Jul. 16, 2026

(54) RNA DEGRADERS AND USES THEREOF

(71) Applicant: Arrakis Therapeutics, Inc., Waltham, MA (US)

(72) Inventors: Robin Prince, Waltham, MA (US); Wilnelly Martinez Ortiz, New York, NY (US); Elias Ndaru, Waltham, MA (US); Lee Roberts, Belmont, MA (US)

(21) Appl. No.: 19/132,496

(22) PCT Filed: Nov. 22, 2023
(86) PCT No.: PCT/US2023/080959

§ 371 Date: May 23, 2025

Related U.S. Application Data
(60) Provisional application No. 63/384,839, filed on Nov. 23, 2022.
(60) Provisional application No. 63/489,644, filed on Mar. 10, 2023.
(60) Provisional application No. 63/499,879, filed on May 3, 2023.
(60) Provisional application No. 63/589,907, filed on Oct. 12, 2023.

(51) Int. Cl. A61K 47/55; A61K 47/54; A61K 47/62

(52) U.S. Cl. CPC A61K 47/55; A61K 47/545; A61K 47/549; A61K 47/62

(57) ABSTRACT

The present invention includes compounds and compositions, and methods of use thereof for modulating an RNA transcript, or a precursor, isoform, fragment, or mutant thereof by degradation of the RNA transcript via recruitment or binding of one or more decay factors (e.g., an RNA binding protein).

### CROSS-REFERENCE TO RELATED APPLICATIONS

This application claims the benefit of U.S. Provisional Application Nos. U.S. 63/589,907, filed Oct. 12, 2023; U.S. 63/499,879, filed May 3, 2023; U.S. 63/489,644, filed Mar. 10, 2023; and U.S. 63/384,839, filed Nov. 23, 2022; the entire contents of each of which are incorporated herein by reference.

### TECHNICAL FIELD OF THE INVENTION

The present invention relates to compounds and methods of use thereof for modulating the activity of RNA transcripts, as well as isoforms, mutants, and fragments thereof, via modulating their degradation and/or otherwise modulating their activity. The invention also provides methods of treating various diseases and conditions mediated by a target RNA transcript, such as those described herein.

### SEQUENCE LISTING

This application contains a Sequence Listing which has been submitted in .xml format via EFS and is hereby incorporated by reference. The ST.26 copy, created on Oct. 26, 2023, is named 394457-013WO_SL.xml, and is 89,934 bytes in size.

### BACKGROUND OF THE INVENTION

RNA, both coding and messenger RNA (mRNA), as well as non-coding RNA (ncRNA), play a multitude of critical regulatory roles in the cell. The total of all RNAs transcribed from DNA—both coding and non-coding—comprise the transcriptome and all cellular biology flows from the transcriptome. All endogenous mammalian diseases are ultimately derived from or modulated by the transcriptome, either directly by RNA or through expressed proteins. Thus, there is the potential to intervene in all human diseases that are protein-mediated or RNA-mediated by modulating the translation or regulatory function of the corresponding mRNAs or ncRNAs.

RNA quality control (QC) mechanisms are varied and ubiquitous. After transcription, RNAs must undergo processing to produce their active forms. RNA processing includes a variety of endo- and exonucleolytic cleavage of sequences at either end of the initial transcript, cleavage of internal sequences (e.g., internal transcribed spacers and introns), nucleotide editing, and various types of functionalization via chemical modification. Notably, most cellular RNAs undergo multiple processing reactions, with alternate pathways (e.g., alternative splicing) leading to distinct products. Multiple RNAs from otherwise similar or identical RNA primary transcripts result in an increase in the functional diversity of RNA and protein species encoded by individual genes.

mRNA decay is the process that causes programmed nucleolytic degradation of the mRNA. The process is enabled by the association of mRNAs with specific RNA-binding proteins (RBPs). Thus, mRNA decay has the potential to directly influence the steady state levels of a translatable pool of mRNAs in vivo. Eukaryotic mRNA decay occurs primarily by enzymatic removal of nucleotides in the 5′-3′ direction and is catalyzed by Xrn1. mRNAs are also degraded in the 3′-5′ direction by the multi-subunit protein complex called the exosome, the catalytic subunit of which is Rrp44. The contribution of 3′-5′ decay to global mRNA turnover is higher in metazoans as compared to lower eukaryotes.

RNA QC mechanisms normally operate to eliminate incorrectly or incompletely processed RNAs. However, if the normal activity of these nucleases and QC pathways could be harnessed to selectively degrade (or not degrade) a disease-causing (or disease-treating) RNA target, it would lead to novel and indeed transformative modes of treating a variety of diseases.

Thus, there is a broad need for agents that selectively inhibit or eradicate target RNAs. The present invention achieves this using bifunctional or chimeric molecules and compositions that both (i) bind to target RNA transcripts and (ii) recruit decay factors, such as RNA-binding proteins (RBPs), that activate an RNA degradation mechanism to degrade the target RNAs or otherwise abrogate the function of the target RNAs (e.g., the availability of the RNA for translation into an active protein). The compounds of this invention and pharmaceutically acceptable compositions thereof meet these requirements and provide other related benefits, as described herein.

### SUMMARY OF THE INVENTION

In one aspect, the present invention provides a compound of Formula B.

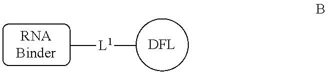

or a pharmaceutically acceptable salt thereof, wherein:
RNA Binder is a moiety that binds to a target RNA transcript;
DFL is a Decay Factor-recruiting Ligand; and
-L¹- is a bivalent linker group that covalently connects the RNA Binder to the DFL;
wherein the DFL binds to or recruits a decay factor.

In some embodiments, the RNA Binder is an oligonucleotide, a polypeptide or an RNA-binding small molecule (rSM). In some embodiments, the RNA Binder is an oligonucleotide. In some embodiments, the RNA Binder is an rSM.

In one aspect, the present invention provides a compound of Formula A:

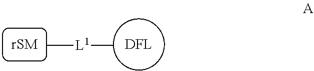

or a pharmaceutically acceptable salt thereof, wherein:
rSM is an RNA-binding small molecule that binds to a target RNA transcript;
DFL is a Decay Factor-recruiting Ligand; and
L¹ is a bivalent linker group that covalently connects the rSM to the DFL;
wherein the DFL binds to or recruits a decay factor.

In some embodiments,

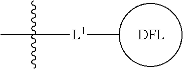

is a compound of Formula I-a:

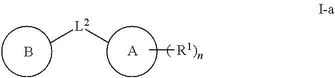

or a pharmaceutically acceptable salt thereof, wherein:
Ring A is a 3-8 membered saturated or partially unsaturated monocyclic carbocyclic ring, phenyl,
an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
Ring B is

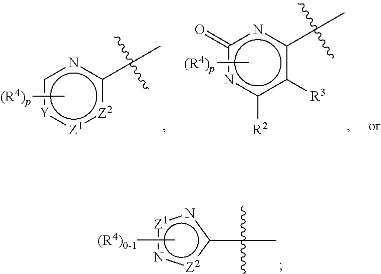

Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is

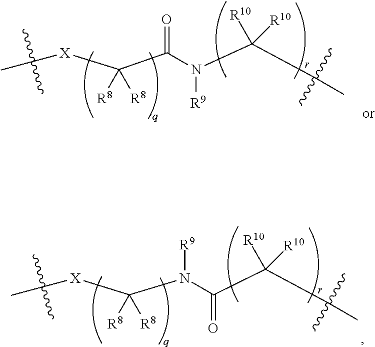

wherein —X— is covalently bound to Ring B; —X— is NR⁶, —O—, —CR⁶R⁷—, or —S—; and one instance of —C(R¹)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or -L¹-;
R⁹ is —R, halogen, —CN, —NC, —C(O)OR, —OC(O)R, —OR, —N(R)₂, or —SR; or R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur; each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R⁹ and R¹⁰ or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-; wherein one and only one of R¹, R², R³, or R⁸ is -L¹- and one end of -L¹- is covalently bound to rSM;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
m is 0, 1, 2, 3, or 4;
n is 0, 1, 2, 3, or 4;
p is 0, 1, 2, or 3;
q is 0, 1, 2, 3, or 4; and
r is 0, 1, 2, 3, or 4.

In some embodiments, Ring A is selected from:

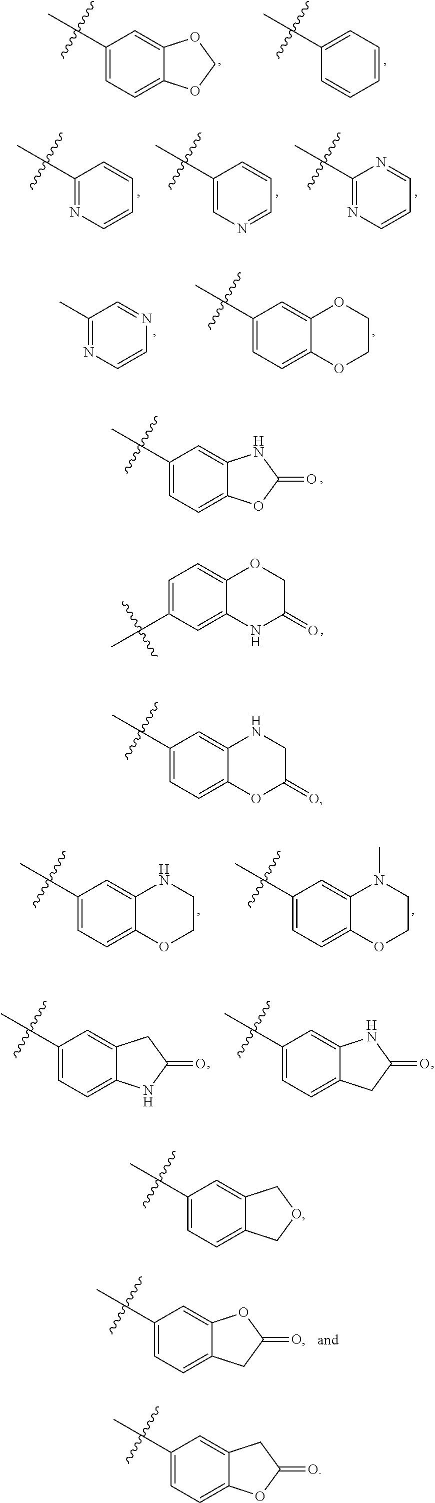

In some embodiments, Ring B is

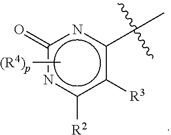

In some embodiments, R¹ is -L¹-.

In some embodiments, R² is -L¹-.

In some embodiments, R³ is -L¹-.

In some embodiments, R⁴ is selected from H, ═O, Me, Et, iPr,

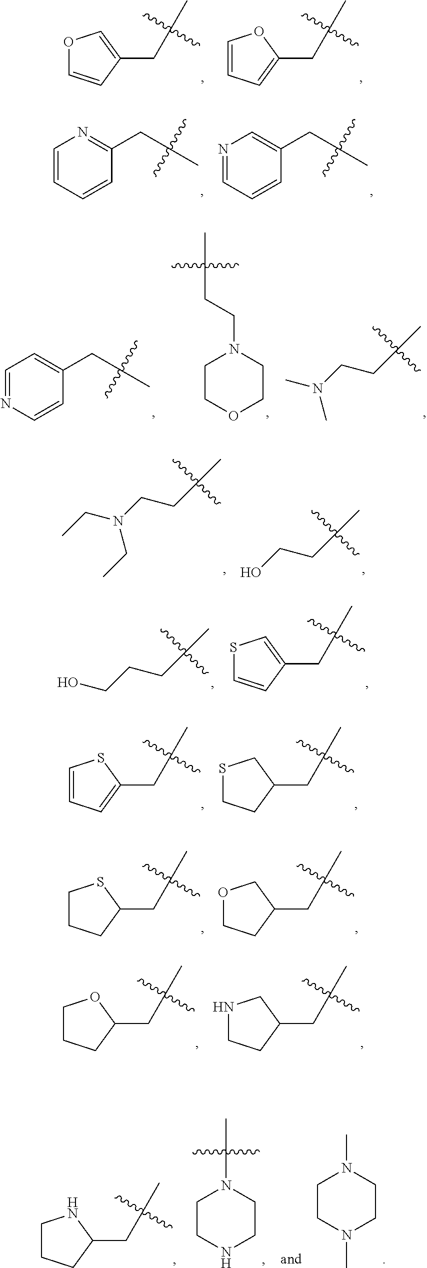

In some embodiments, R⁸ is -L¹-.

In some embodiments, -L²- is selected from

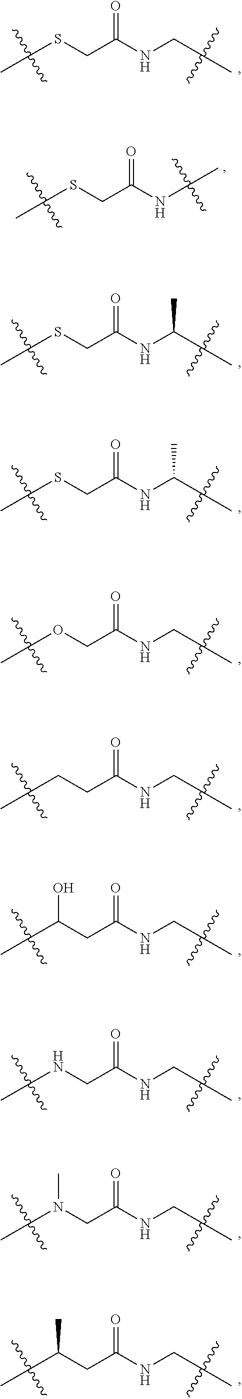

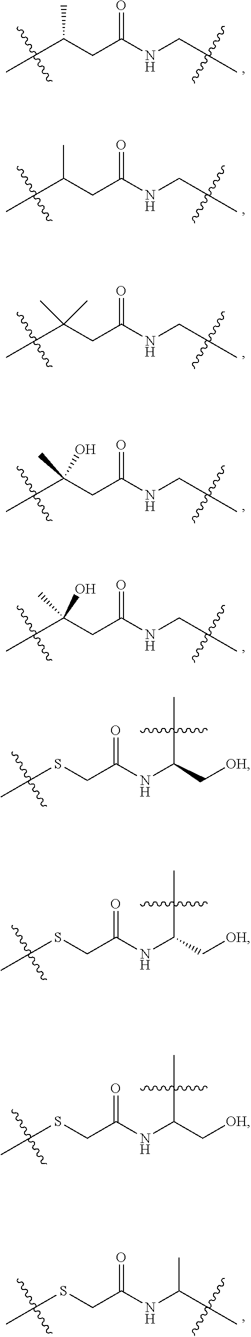

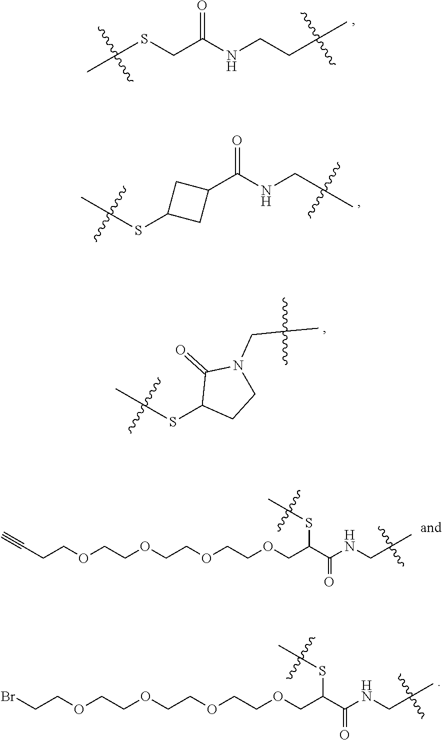 [SMILES: *CCNC(=O)CS* ; *CNC(=O)C1CC(S*)C1 ; *CN1CCC(S*)C1=O ; *CNC(=O)C(COCCOCCOCCOCCC#C)S* ; *CNC(=O)C(COCCOCCOCCOCCBr)S*]

In some embodiments, the compound is of Formula II-a or II-b:

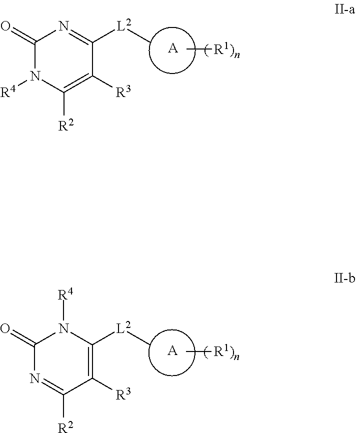

or a pharmaceutically acceptable salt thereof.

In some embodiments, the compound is of Formula IIIa:

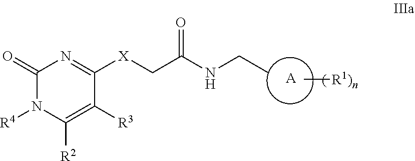

or a pharmaceutically acceptable salt thereof.

In some embodiments, the compound is of Formula IV-a

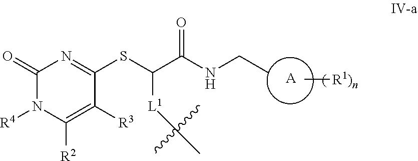

or a pharmaceutically acceptable salt thereof.

In some embodiments,

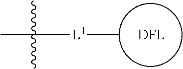

is a compound of Formula I-c:

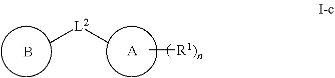

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur,
Ring B is

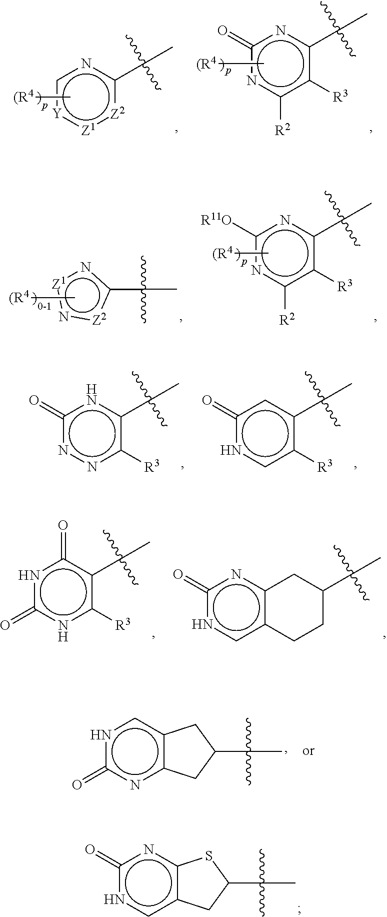

Y is N or CH;
Z¹ is N, C═O or CR²;
Z² is N, C═O, or CR³; provided that Z¹ and Z² are not both N or C═O; each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is

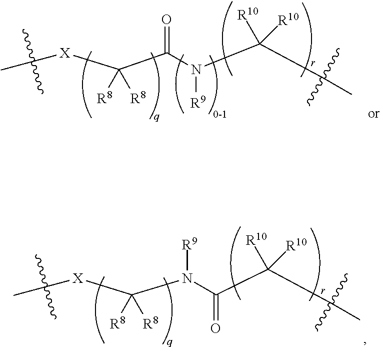

wherein —X— is covalently bound to Ring B; —X— is a bond, —NR⁶, —O—, —CR⁶R⁷—, —C(O)—, —S—, or —S(O)₂—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or two R⁸, taken together with the carbon atom to which they are attached, form a 3-6 membered carbocyclic ring;
R⁹ is —R, halogen, —CN, —NC, —C(O)OR, —OC(O)R, —OR, —N(R)₂, or —SR; or R⁸ and R⁹, taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur;
each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or two R¹⁰, taken together with the carbon atom to which they are attached, form a 3-6 membered carbocyclic ring; or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a bivalent linker group that covalently connects the RNA Binder to the DFL; wherein one and only one of R¹, R², R³, R⁸, or R¹¹ is -L¹- and one end of -L¹- is covalently bound to the RNA Binder;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
R¹¹ is H, C₁₋₃ alkyl, or -L¹-;
m is 0, 1, 2, 3, or 4;
n is 0, 1, 2, 3, 4, or 5;
p is 0, 1, 2, or 3;
q is 0, 1, 2, 3, or 4; and
r is 0, 1, 2, 3, or 4.

In some embodiments, Ring A is selected from:

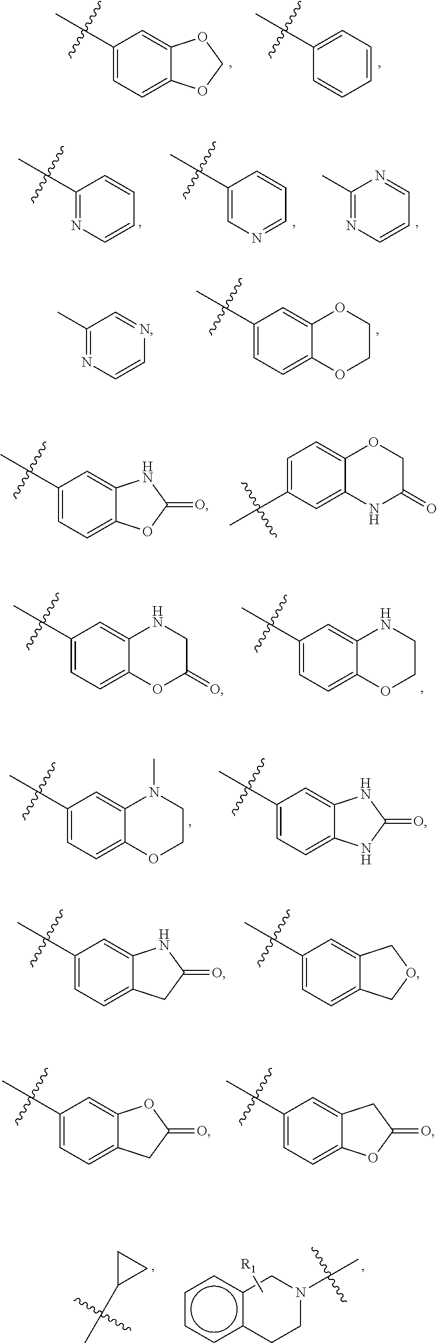

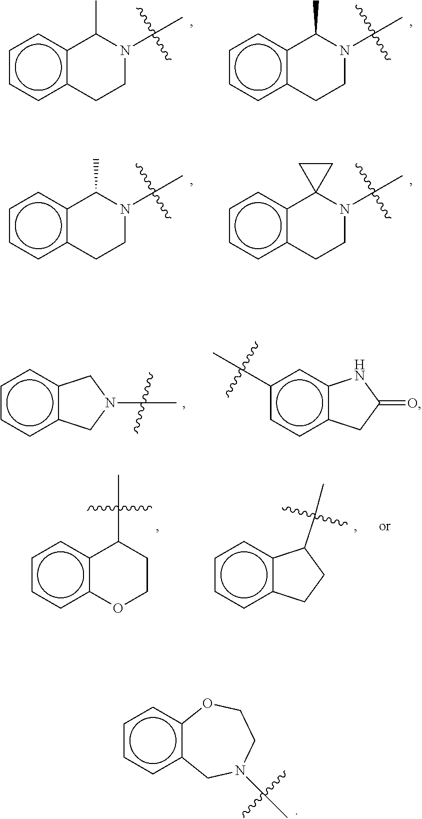

In some embodiments, Ring B is

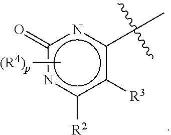

In some embodiments, R¹ is -L¹-.

In some embodiments, R² is -L¹-.

In some embodiments, R³ is -L¹-.

In some embodiments, R⁴ is selected from H, ═O, Me, Et, iPr

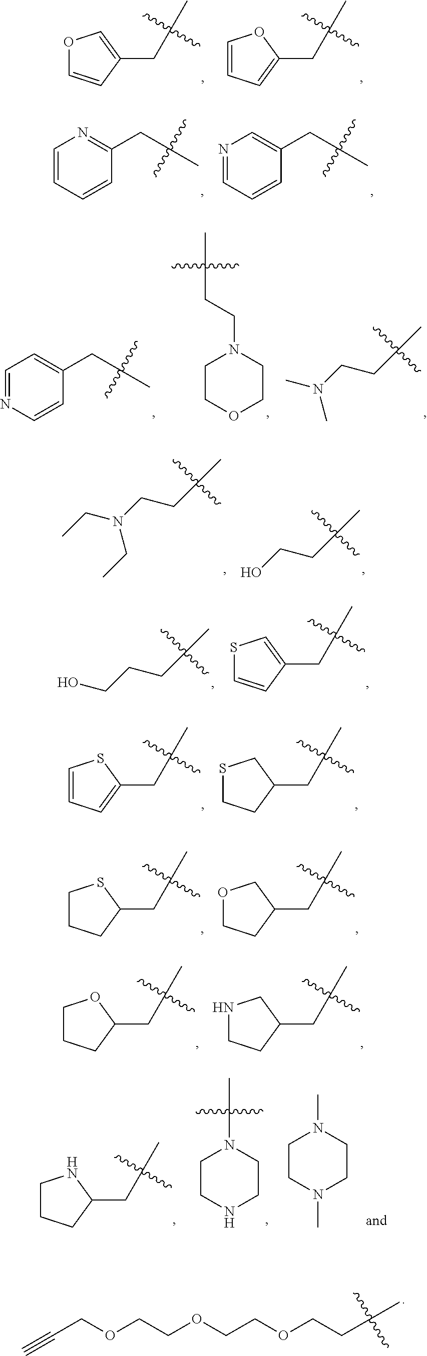

In some embodiments, R⁸ is -L¹-.

In some embodiments, R¹¹ is H, C₁₋₃ alkyl, or -L¹-.

In some embodiments, -L²- is selected from

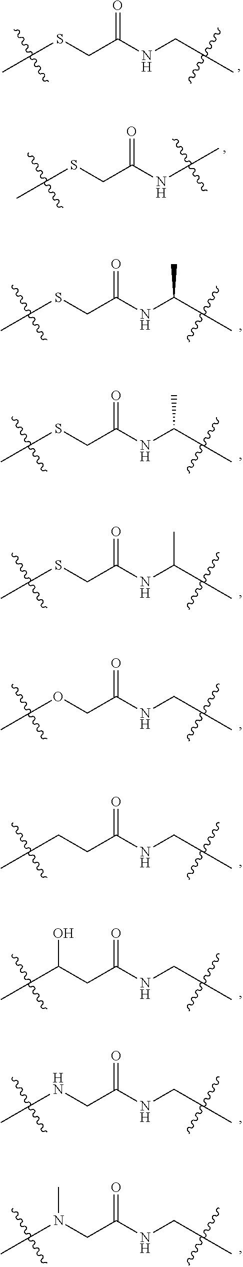

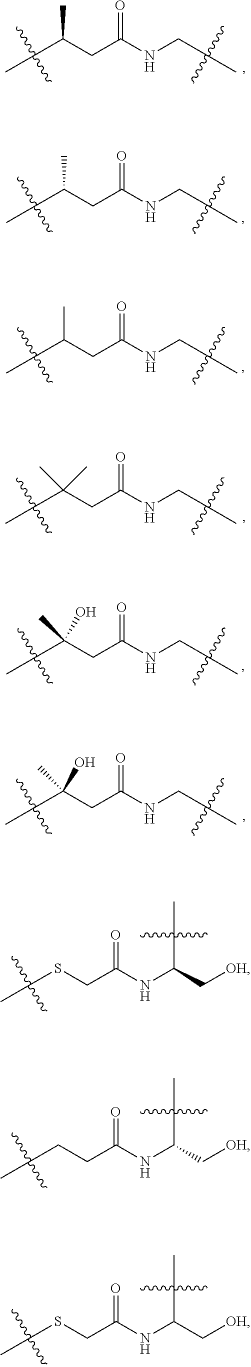

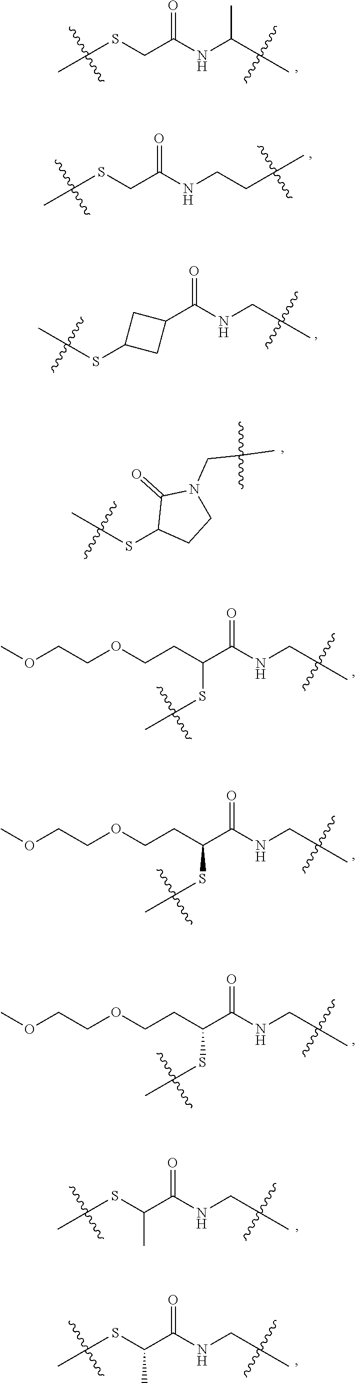

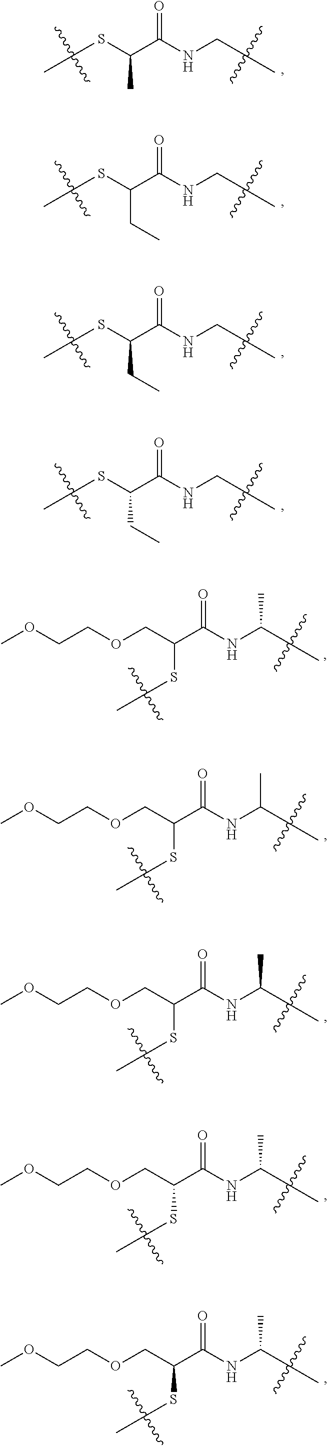

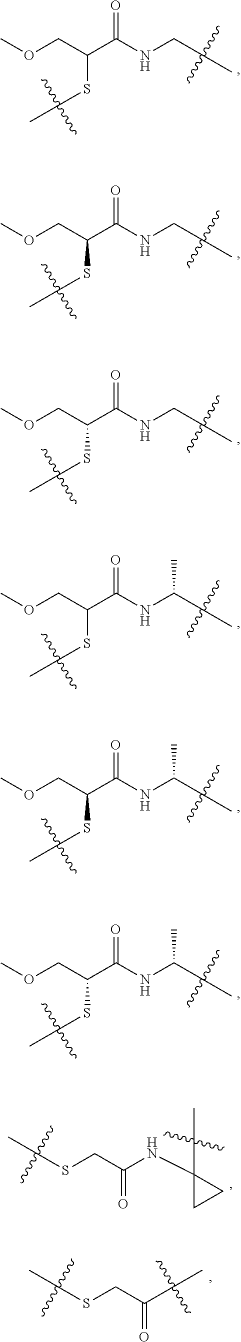

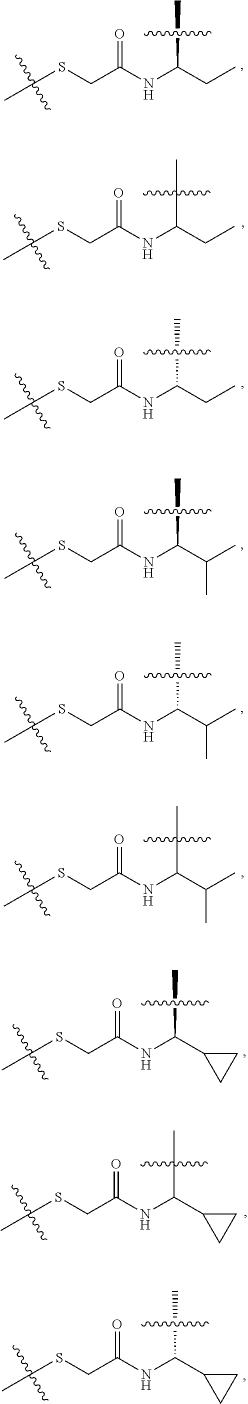

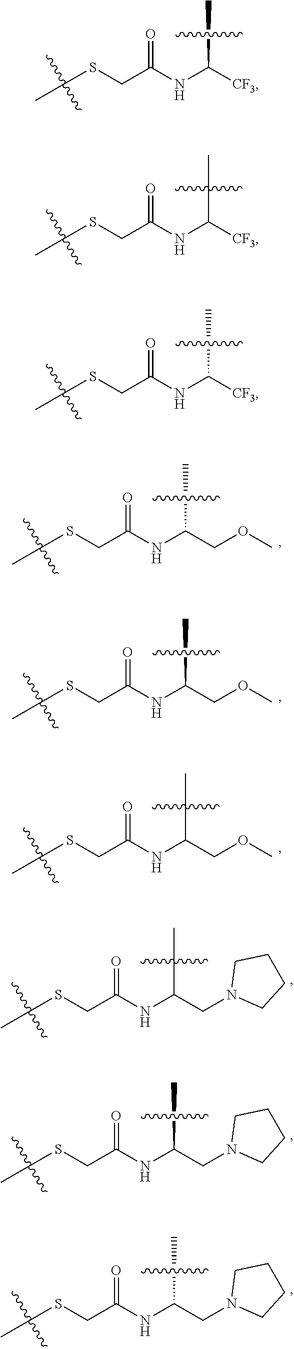

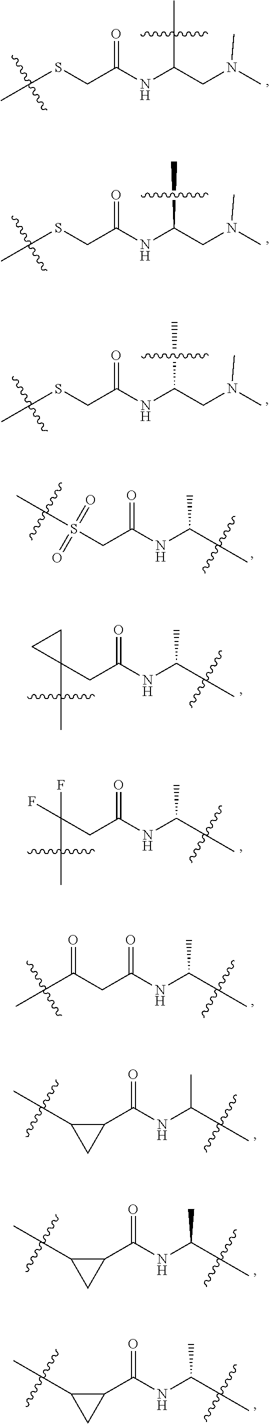

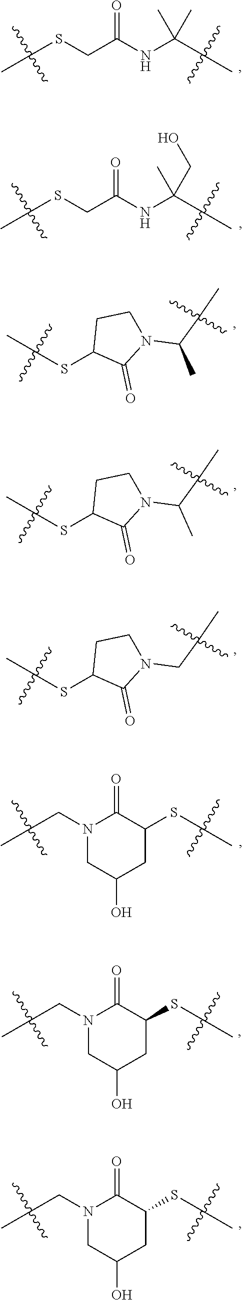

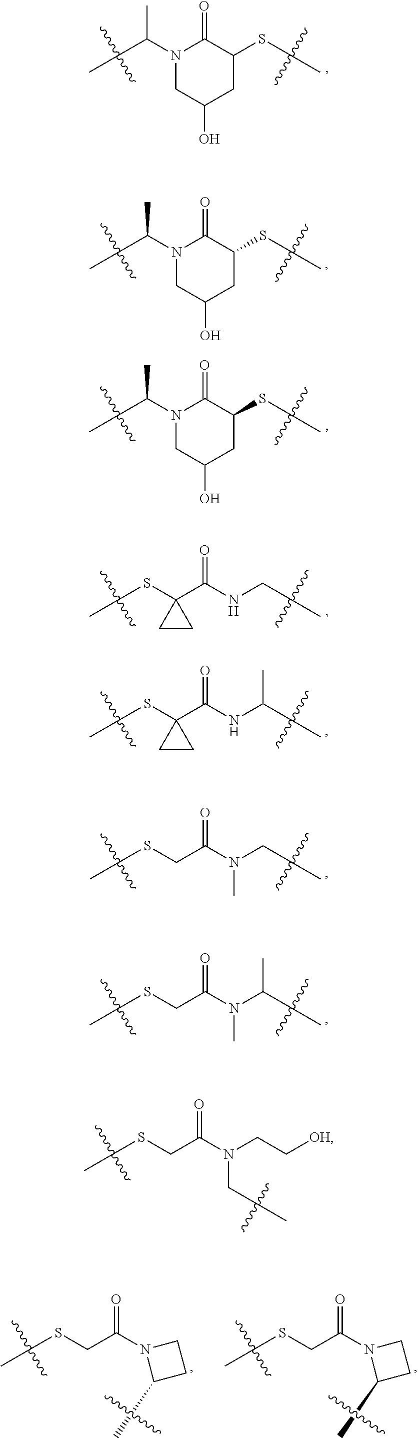

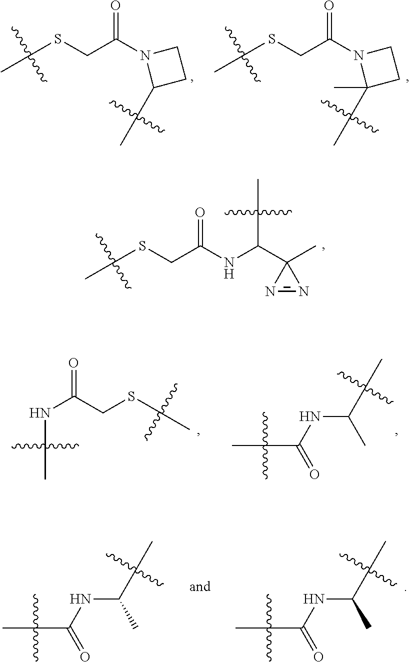

In some embodiments, the compound is of Formula IX-a, IX-b, IX-c, XVI-a, XVI-b or XVI-c:

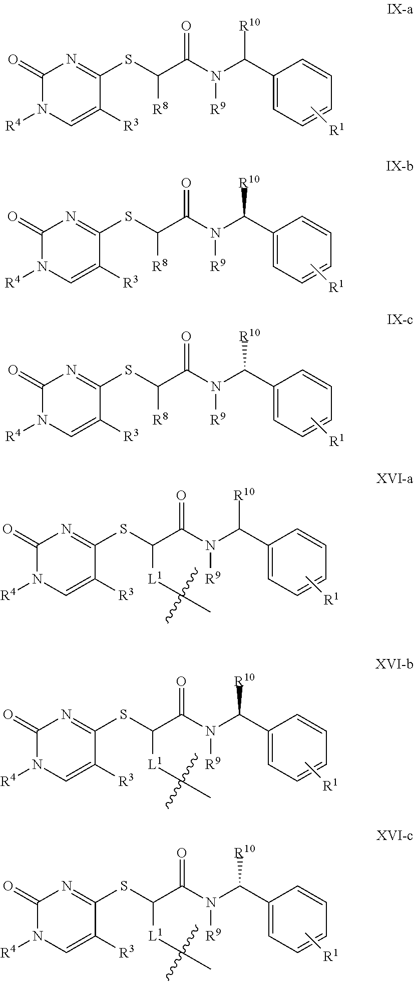

or a pharmaceutically acceptable salt thereof.

In some embodiments, the compound is of Formula X-a, X-b, X-c, XVII-a, XVII-b, or XVII-c:

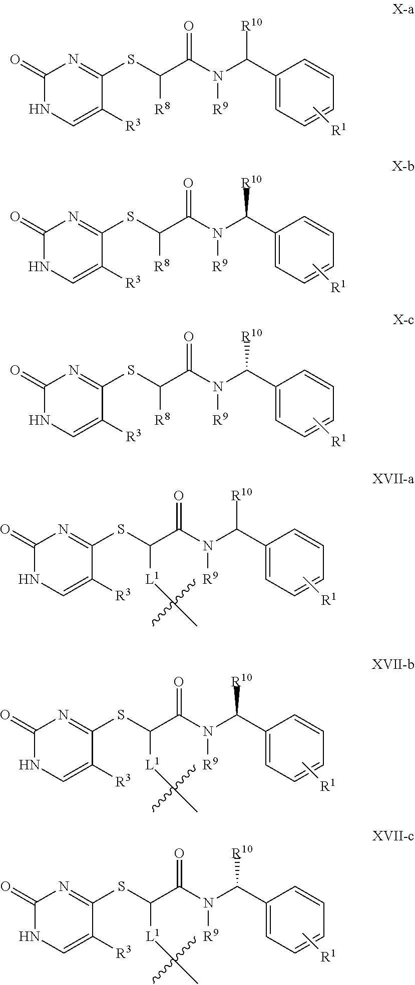

or a pharmaceutically acceptable salt thereof.

In some embodiments, the compound is of Formula XI-a, XI-b, XI-c, XVIII-a, XVIII-b, or XVIII-c:

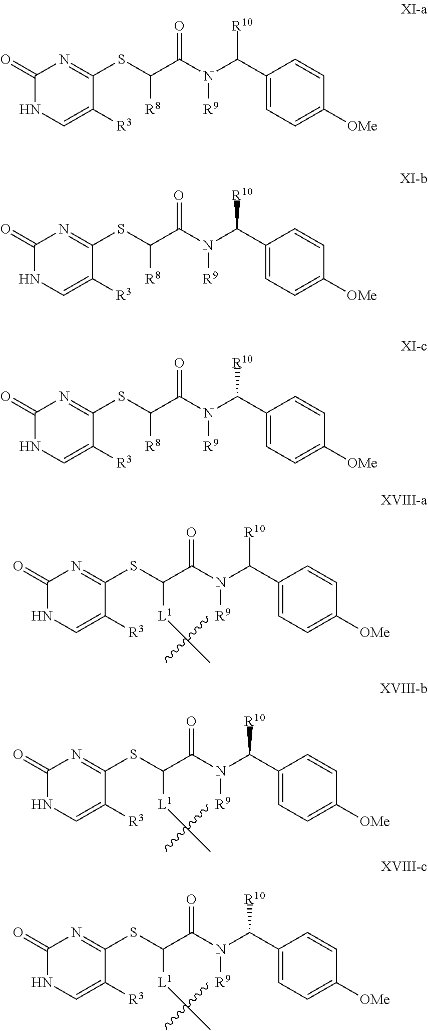

or a pharmaceutically acceptable salt thereof.

In some embodiments, the compound is of Formula XII-a, XII-b, XII-c, XIX-a, XIX-b or XIX-c:

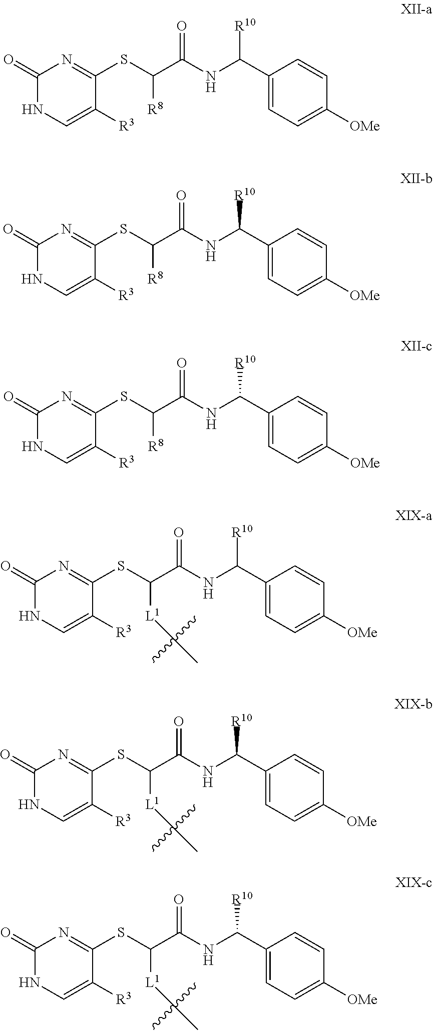

or a pharmaceutically acceptable salt thereof.

In some embodiments, the compound is of Formula XTII-a, XIII-b, XIII-c, XX-a, XX-b or XX-c:

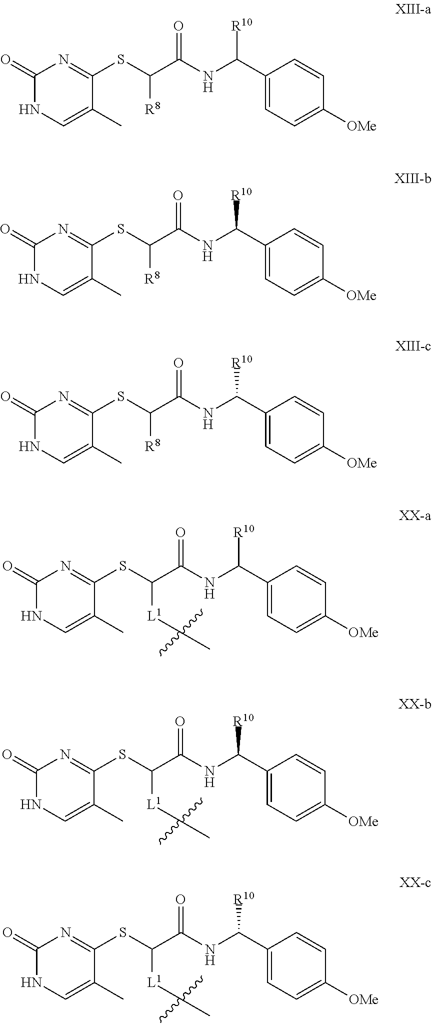

or a pharmaceutically acceptable salt thereof.

In some embodiments, the compound is of Formula XIV-a, XIV-b XIV-c, XXI-a, XXI-b or XXI-c:

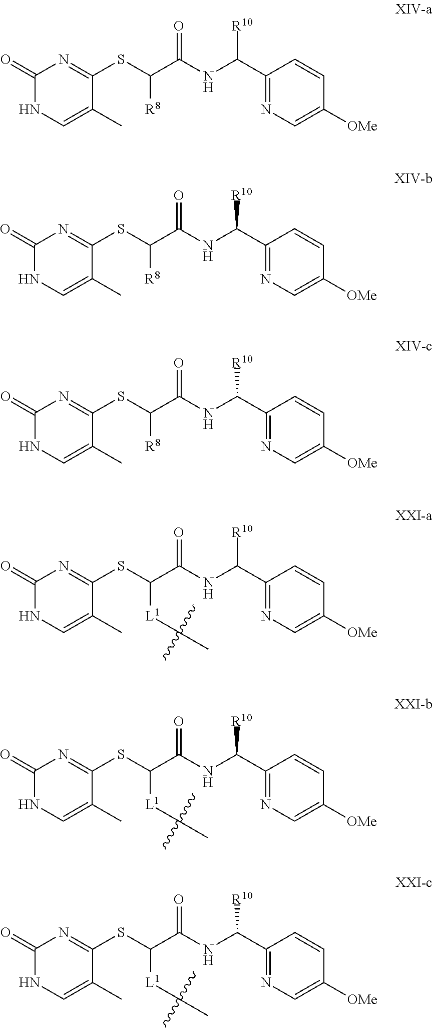

or a pharmaceutically acceptable salt thereof.

In some embodiments, the compound is of Formula XV-a or XXII-a:

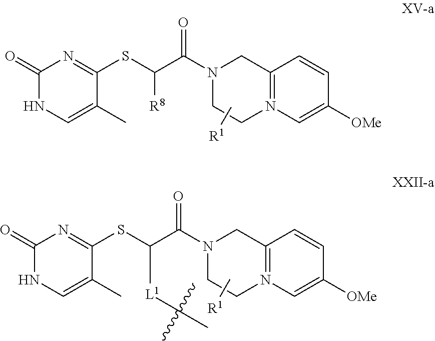

or a pharmaceutically acceptable salt thereof.

In some embodiments, the compound is of Formula XXIII-a, Formula XXIII-b or Formula XXIII-c:

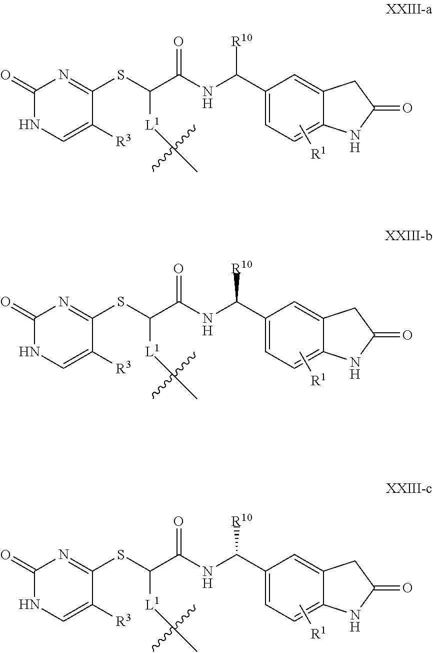

or a pharmaceutically acceptable salt thereof.

In some embodiments, the compound is of Formula XXIII-d, Formula XXIII-e or Formula XXIII-f:

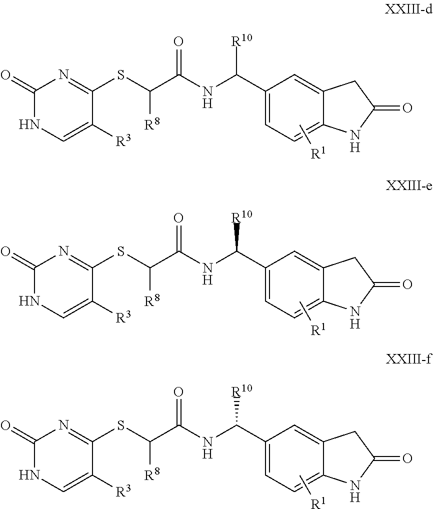

or a pharmaceutically acceptable salt thereof.

In some embodiments, the compound is of Formula XXIV-a:

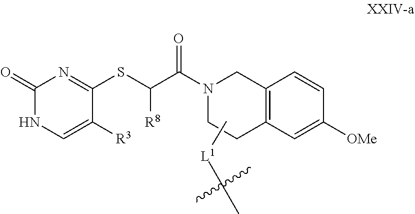

or a pharmaceutically acceptable salt thereof.

In some embodiments, the compound is of Formula XXIV-b:

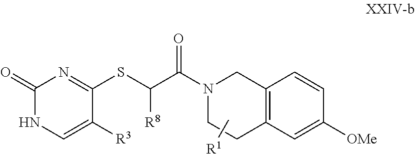

or a pharmaceutically acceptable salt thereof.

In some embodiments, the compound is of Formula XXV-a:

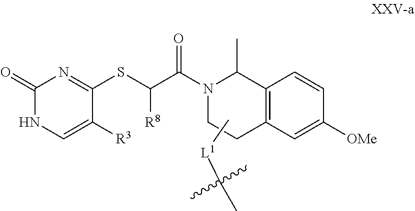

or a pharmaceutically acceptable salt thereof.

In some embodiments, the compound is of Formula XXV-b:

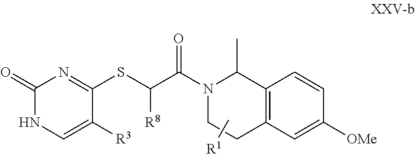

or a pharmaceutically acceptable salt thereof.

In some embodiments, the decay factor is a protein that binds or interacts with RNA (an RBP) and wherein the interaction of the RBP with the RNA leads to modulation of the target RNA transcript in vivo.

In some embodiments, the RBP is part of the CCR4-NOT (Carbon Catabolite Repression-Negative On TATA-less) complex.

In some embodiments, the RBP is CNOT7.

In some embodiments, the DFL does not bind to the active site of CNOT7.

In some embodiments, the DFL binds CNOT7 without abrogating the enzymatic activity of the CNOT7 and/or the CCR4-NOT complex.

In some embodiments, the target RNA transcript is an mRNA or a precursor, isoform, unspliced isoform, splicing intermediate, fragment, or mutant thereof.

In some embodiments, the target RNA transcript is selected from one of those listed in Table C or D; or a precursor, isoform, unspliced isoform, splicing intermediate, fragment, or mutant thereof.

In some embodiments, the rSM is selected from any one of those described in the section entitled exemplary rSMs.

In some embodiments, the rSM is one of those shown in Table 2.

In some embodiments, the present invention provides a pharmaceutical composition comprising the compound described herein, or a pharmaceutically acceptable salt thereof, and a pharmaceutically acceptable carrier.

Another aspect of the present invention provides a method of modifying the amount of a protein in a cell, the method comprising administering the compound or composition described herein, or a pharmaceutically acceptable salt thereof, that acts on a target RNA transcript or a precursor, isoform, fragment, or mutant thereof, in an amount sufficient to modify the amount of the protein in the cell.

In some embodiments, modifying the amount of a protein in a cell is reducing the amount of protein in the cell.

Another aspect of the present invention provides a method of modulating the availability for protein translation of a target RNA transcript or a precursor, isoform, fragment, or mutant thereof, comprising contacting the target RNA transcript or a precursor, isoform, fragment, or mutant thereof with the compound or composition described herein, or a pharmaceutically acceptable salt thereof, that binds to the target RNA transcript or an isoform, fragment, or mutant thereof.

Another aspect of the present invention provides a method of modulating the translation of a target protein or mutant thereof, comprising contacting a target RNA transcript or a precursor, isoform, fragment, or mutant thereof with the compound or composition described herein, or a pharmaceutically acceptable salt thereof.

Another aspect of the present invention provides a method of decreasing the half-life or increasing degradation of a target RNA transcript or a precursor, isoform, fragment, or mutant thereof, comprising contacting the target RNA transcript or the precursor, isoform, fragment, or mutant thereof with the compound or composition described herein, or a pharmaceutically acceptable salt thereof.

Another aspect of the present invention provides a method of treating a disease, comprising administering to a subject in need thereof the compound or composition described herein, or a pharmaceutically acceptable salt thereof.

In some embodiments, the disease is characterized by an aberrant level of a protein in a cell.

In some embodiments, the disease is one of those listed in Table C or D.

In some embodiments, the disease is a cancer.

In some embodiments, the RBP is CNOT7.

### BRIEF DESCRIPTION OF THE DRAWINGS

FIG. 1 shows a schematic of the CCR4-NOT complex, the major mediator of deadenylation.

FIG. 2 shows an exemplary compound docked to the CNOT7 protein. Exit vectors that allow for the attachment of a linker are shown.

FIG. 3 shows the binding of compound I-1 to CNOT7 as determined by SPR.

FIG. 4 shows the binding of selected compounds to CNOT7 as determined by SPR.

FIG. 5 provides a schematic of a surface plasmon resonance (SPR) experiment to interrogate if a CNOT7 binding compound binds in the active site of CNOT7.

FIG. 6 shows exemplary results of an AMP competition assay by SPR to investigate CNOT7 binding characteristics.

FIG. 7 shows exemplary results of an AMP competition assay by SPR to investigate CNOT7 binding characteristics.

FIG. 8 shows the degradation of RNA by CNOT7 and CNOT7 mutant proteins.

FIG. 9 shows a schematic of biochemical assays to evaluate bifunctional degraders. Included therein is SEQ ID NO: 40.

FIG. 10 shows increased RNA degradation through the action of bifunctional degraders.

FIG. 11 shows that CNOT7 binders do not abrogate CNOT7 functioning.

FIG. 12 shows a schematic of reporter constructs for cellular bifunctional degrader assays.

FIG. 13 shows a schematic of a cellular assay to evaluate bifunctional degraders.

FIG. 14 shows the intracellular degradation of RNA through the action of CNOT7.

FIG. 15 shows a schematic of a cellular assay to evaluate bifunctional degraders for the degradation of an endogenous protein.

FIG. 16 shows the degradation of endogenous RNA through the action of CNOT7.

FIG. 17 shows the structures of I-329 and I-371.

FIG. 18 shows heterobifunctional molecules that can facilitate the degradation of RNA by CNOT7.

FIG. 19A shows heterobifunctional molecules that can facilitate the degradation of RNA by CNOT7. FIG. 19B shows heterobifunctional molecules that can facilitate the degradation of RNA by CNOT7.

FIG. 20 shows additional biochemical degrader data for compounds I-371 and I-397.

FIGS. 21A and 21B show the results of a CNOT7 inhibition assay. Two of the three CNOT7 binding moieties (i.e., baits) that were evaluated (I-243, and I-330) slightly inhibited CNOT7 activity, while the CNOT7 binding moiety I-198 did not inhibit CNOT7 activity.

FIG. 22 shows the results of a CNOT7:CNOT1 recombinant assay. The data show that neither the CNOT7 binding moieties (i.e., baits) that were evaluated nor the three CNOT7 heterobifunctionals, in which the CNOT7 binding moiety was linked to an rSM (I-317, I-496 and ARK I-397) inhibited or accelerated the deadenylation activity of the CNOT7:CNOT1 complex. As expected, the known CNOT7 active site binder Compound A (See FIG. 11) was able to inhibit the CNOT7 activity. It should be noted that the CCR4NOT complex includes proteins other than CNOT7, for instance CNOT6, that have deadenylation activity.

FIGS. 23A and 23B show the results of a biochemical AMP-Glo assay to measure deadenylation. The data show that AMP increases over the time course in all conditions for the heterobifunctional compounds, as expected, and that more AMP is generated for the heterobifunctional compounds compared to DMSO background.

FIG. 24 shows results from a stable CNOT7 cell line assay (see Example 23).

FIG. 25 shows results from a PC9 HiBiT cell line assay (see Example 24).

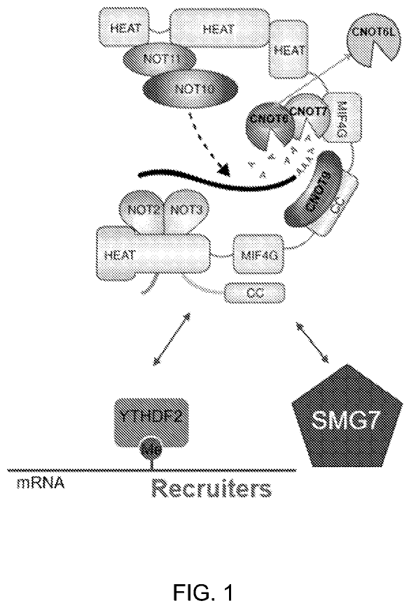

### DETAILED DESCRIPTION OF CERTAIN EMBODIMENTS

### 1. General Description of Certain Embodiments of the Invention; Definitions

In one aspect, the present invention provides a bifunctional compound of Formula B:

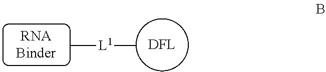

or a pharmaceutically acceptable salt thereof, wherein:
RNA Binder is a moiety that binds to a target RNA transcript;
DFL is a Decay Factor-recruiting Ligand; and
L¹- is a bivalent linker group that covalently connects the RNA Binder to the DFL;
wherein the DFL binds to or recruits a decay factor;
wherein the DFL binds to or recruits one or more decay factors that degrade the target RNA transcript.

In some embodiments, the RNA binder is an oligonucleotide, peptide, oligosaccharide or an RNA-binding small molecule (rSM). In some embodiments, the RNA binder is an oligonucleotide. In some embodiments, the RNA binder is an rSM. In some embodiments, the DFL binds an RBP. In some embodiments, the present invention provides a bifunctional composition comprising an RNA binder and a DFL useful as a modulator of targeted degradation of a variety of target RNA transcripts, which are then degraded and/or otherwise inhibited by the bifunctional composition as described herein. An advantage of the composition provided herein is that a broad range of pharmacological activities is possible, consistent with the degradation/inhibition of a target RNA transcript from virtually any RNA class or family.

In some embodiments, the composition includes an RNA binder, such as an oligonucleotide, and the composition binds the RNA through its oligonucleotide. Oligonucleotides that bind RNA are well known. Generally, the oligonucleotide that binds the target RNA will have a nucleic acid sequence that is complementary to a nucleic acid sequence in the target RNA. The binding of an oligonucleotide with a complimentary sequence to a target RNA sequence is stable and highly specific. In some embodiments, the composition including an RNA binder, such as an oligonucleotide, is optimized for intracellular delivery. Optimization of oligonucleotides and compositions comprising oligonucleotides for intracellular delivery is well established.

In some embodiments, the composition comprises an RNA binder. In some embodiments, the RNA binder is an oligonucleotide. In some embodiments, the oligonucleotide can specifically bind an RNA target. In some embodiments, the oligonucleotide comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. In some embodiments, the oligonucleotide consists of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. In some embodiments, the oligonucleotide has been modified for therapeutic delivery.

In some embodiments, the oligonucleotide is an antisense oligonucleotide (ASO). In some embodiments, the ASO is a therapeutic ASO. Non-limiting examples of therapeutic ASOs include Mipomersen, Custirsen, Fomivirsen, Oblimersen, Eteplirsen, Nusinersen, Inotersen, Givosiran, Golodirsen and Viltolarsen.

In one aspect, the present invention provides a bifunctional compound of Formula A:

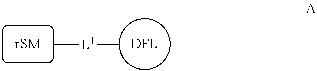

or a pharmaceutically acceptable salt thereof, wherein:
rSM is an RNA-binding small molecule that binds to a target RNA transcript;
DFL is a Decay Factor-recruiting Ligand; and
-L¹- is a bivalent linker group that covalently connects the rSM to the DFL;
wherein the DFL binds to or recruits one or more decay factors that degrade the target RNA transcript.

In some embodiments,

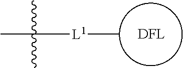

is a compound of Formula I-a:

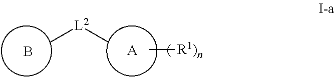

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
Ring B is

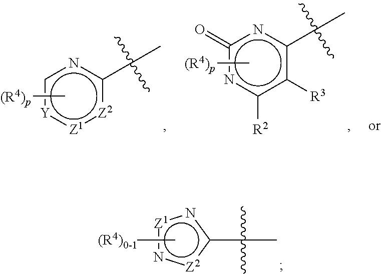

Y is N or CH;
Z¹ is N, C═O or CR²;
Z² is N, C═O, or CR³; provided that Z¹ and Z² are not both N or C═O; each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R,
-L²- is

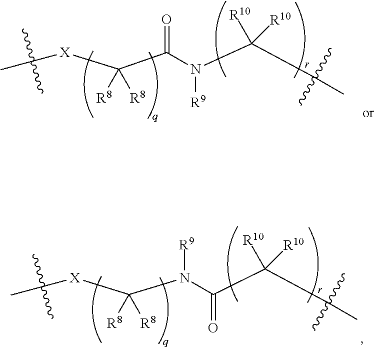

wherein —X— is covalently bound to Ring B; —X— is NR⁶, —O—, —CR⁶R⁷—, or —S—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or -L¹-;
R⁹ is —R, halogen, —CN, —NC, —C(O)OR, —OC(O)R, —OR, —N(R)₂, or —SR; or R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur; each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R⁸ and R¹⁰ or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-; wherein one and only one of R¹, R², R³, or R⁸ is -L¹- and one end of -L¹- is covalently bound to rSM;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
m is 0, 1, 2, 3, or 4;
n is 0, 1, 2, 3, or 4;
p is 0, 1, 2, or 3;
q is 0, 1, 2, 3, or 4; and
r is 0, 1, 2, 3, or 4.

As defined generally above, Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, Ring A is a 3-8 membered saturated or partially unsaturated monocyclic carbocyclic ring. In some embodiments, Ring A is phenyl. In some embodiments, Ring A is an 8-10 membered bicyclic aromatic carbocyclic ring. In some embodiments, Ring A is a 4-8 membered saturated or partially unsaturated monocyclic heterocyclic ring having 1-3 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is an 8-10 membered bicyclic heteroaromatic ring having 1-5 heteroatoms independently selected from nitrogen, oxygen, and sulfur.

In some embodiments, Ring A is selected from:

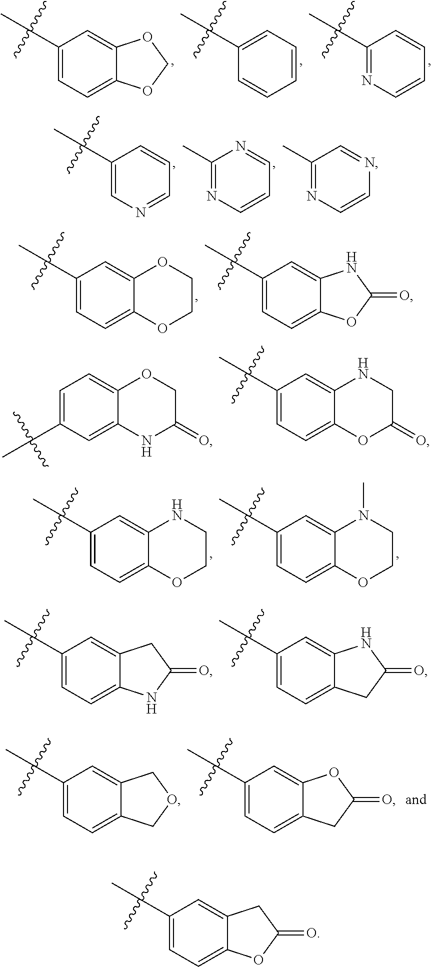

In some embodiments, Ring A is selected from

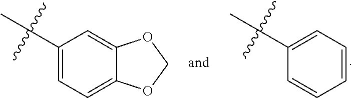 [SMILES: *c1ccc2c(c1)OCO2 ; *c1ccccc1]

In some embodiments, Ring A is selected from those depicted in Table 1, below.

As defined generally above, Ring B is

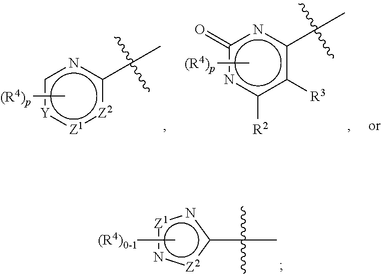

In some embodiments, Ring B is

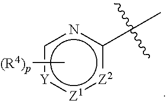

In some embodiments, Ring B is

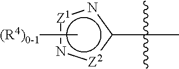

In some embodiments, Ring B is

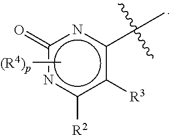

In some embodiments, Ring B is

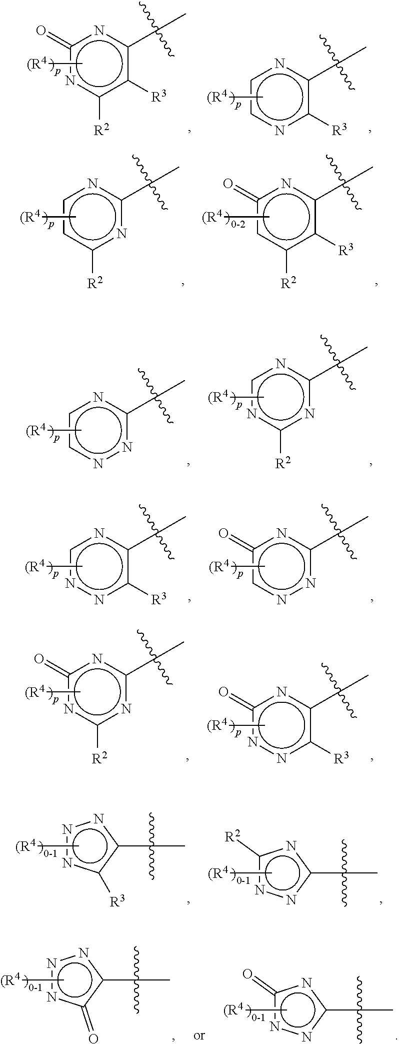

In some embodiments, Ring B is selected from

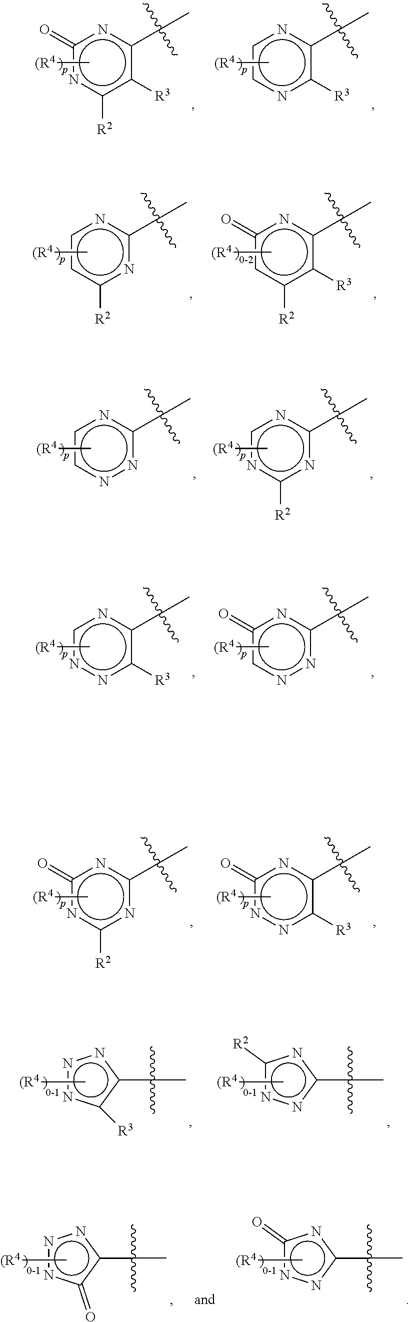

In some embodiments, Ring B is

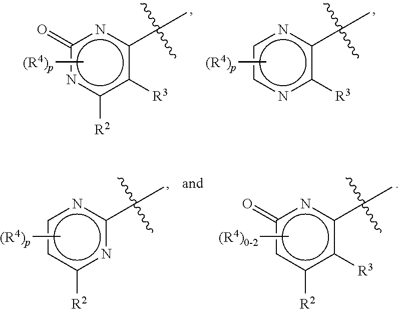

In some embodiments, Ring B is selected from

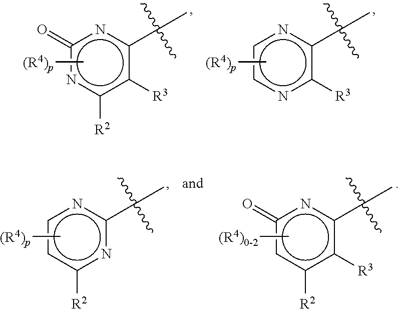

In some embodiments, Ring B is

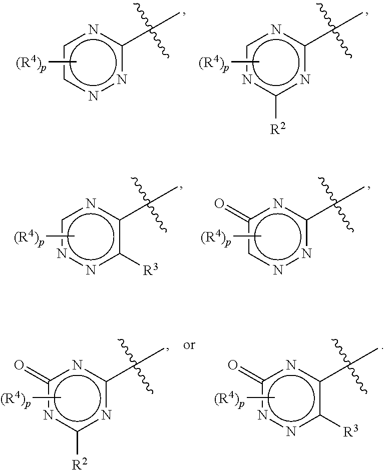

In some embodiments, Ring B is selected from

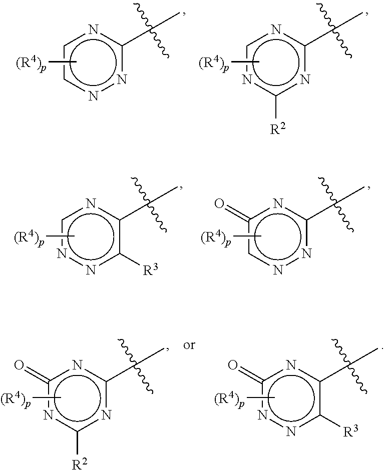

In some embodiments, Ring B is

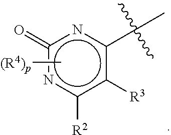

In some embodiments, Ring B is

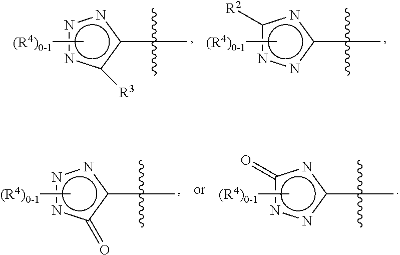

In some embodiments, Ring B is selected from

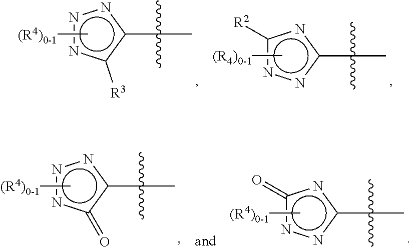

In some embodiments, Ring B is selected from those depicted in Table 1, below.

As defined generally above, Y is N or CH.

In some embodiments, Y is N. In some embodiments, Y is CH.

In some embodiments, Y is CH. In some embodiments, Y is substituted with R⁴.

In some embodiments, Y is selected from those depicted in Table 1, below.

As defined generally above, Z¹ is N, C═O, or CR².

In some embodiments, Z¹ is N. In some embodiments, Z¹ is C═O. In some embodiments, Z¹ is CR².

In some embodiments, Z¹ is selected from those depicted in Table 1, below.

As defined generally above, Z² is N, C═O, or CR³.

In some embodiments, Z² is N. In some embodiments, Z² is C═O. In some embodiments, Z² is CR³.

In some embodiments, Z² is selected from those depicted in Table 1, below.

As defined generally above, each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-.

In some embodiments, R¹ is R. In some embodiments, R¹ is halogen. In some embodiments, R¹ is —CN. In some embodiments, R¹ is —NC. In some embodiments, R¹ is —C(O)OR. In some embodiments, R¹ is —OC(O)R. In some embodiments, R¹ is —C(O)N(R)₂. In some embodiments, R¹ is —N(R)C(O)R. In some embodiments, R¹ is —N(R)C(O)N(R)₂. In some embodiments, R¹ is —OC(O)N(R)₂. In some embodiments, R¹ is —N(R)C(O)OR. In some embodiments, R¹ is —OR. In some embodiments, R¹ is —N(R)₂. In some embodiments, R¹ is —NO₂. In some embodiments, R¹ is —N₃. In some embodiments, R¹ is —SR. In some embodiments, R¹ is —S(O)R. In some embodiments, R¹ is —S(O)₂R. In some embodiments, R¹ is —S(O)₂N(R)₂. In some embodiments, R¹ is —NRS(O)₂R. In some embodiments, R¹ is -L¹-.

In some embodiments, R¹ is hydrogen. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, and sulfur. In some embodiments, R¹ is an optionally substituted 5-6 membered monocyclic heteroaromatic ring having 1-4 heteroatoms independently selected from nitrogen, oxygen, and sulfur. In some embodiments, R¹ is an optionally substituted 8-10 membered bicyclic heteroaromatic ring having 1-5 heteroatoms independently selected from nitrogen, oxygen, and sulfur.

In some embodiments, R¹ is a C₁₋₈ bivalent straight or branched hydrocarbon chain. In some embodiments, R¹ is a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R¹ is selected from H, F, Cl, OH, Me, Et, i-Pr,

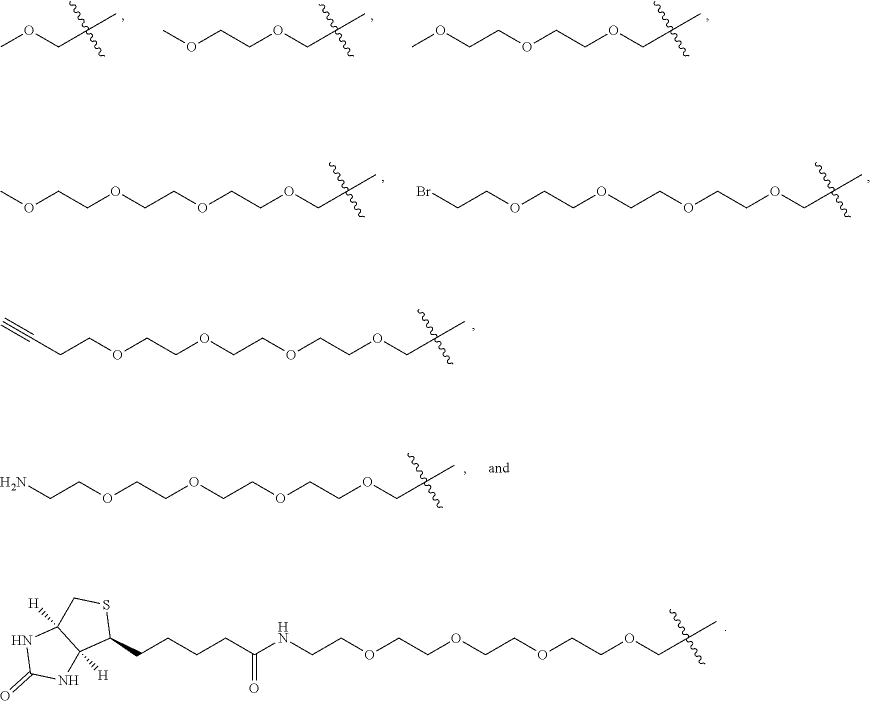

In some embodiments, R¹ is selected from those depicted in Table 1, below.

As defined generally above, each R² and R³ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² is R. In some embodiments, R² is halogen. In some embodiments, R² is —CN. In some embodiments, R² is —NC. In some embodiments, R² is —C(O)OR. In some embodiments, R² is —OC(O)R. In some embodiments, R² is —C(O)N(R)₂. In some embodiments, R² is —N(R)C(O)R. In some embodiments, R² is —N(R)C(O)N(R)₂. In some embodiments, R² is —OC(O)N(R)₂. In some embodiments, R² is —N(R)C(O)OR. In some embodiments, R² is —OR. In some embodiments, R² is —N(R)₂. In some embodiments, R² is —NO₂. In some embodiments, R² is —N₃. In some embodiments, R² is —SR. In some embodiments, R² is —S(O)R. In some embodiments, R² is —S(O)₂R. In some embodiments, R² is —S(O)₂N(R)₂. In some embodiments, R² is —NRS(O)₂R. In some embodiments, R² is -L¹-.

In some embodiments, R² is hydrogen. In some embodiments, R² is an optionally substituted C₁₋₆ aliphatic group. In some embodiments, R² is an optionally substituted 4-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, and sulfur. In some embodiments, R² is an optionally substituted 5-6 membered monocyclic heteroaromatic ring having 1-4 heteroatoms independently selected from nitrogen, oxygen, and sulfur. In some embodiments, R² is an optionally substituted 8-10 membered bicyclic heteroaromatic ring having 1-5 heteroatoms independently selected from nitrogen, oxygen, and sulfur.

In some embodiments, R² is a C₁₋₈ bivalent straight or branched hydrocarbon chain. In some embodiments, R² is a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R² is selected from H, F, Cl, OH, Me, Et, i-Pr,

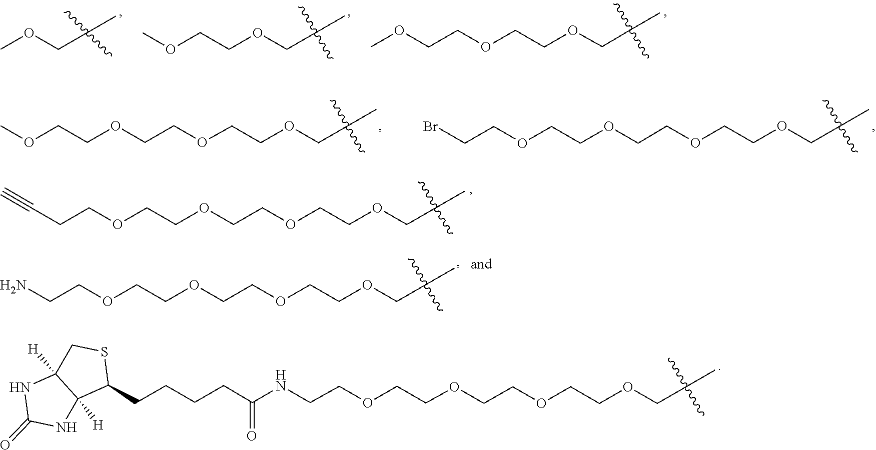

In some embodiments, R³ is R. In some embodiments, R³ is halogen. In some embodiments, R³ is —CN. In some embodiments, R³ is —NC. In some embodiments, R³ is —C(O)OR. In some embodiments, R³ is —OC(O)R. In some embodiments, R³ is —C(O)N(R)₂. In some embodiments, R³ is —N(R)C(O)R. In some embodiments, R³ is —N(R)C(O)N(R)₂. In some embodiments, R³ is —OC(O)N(R)₂. In some embodiments, R³ is —N(R)C(O)OR. In some embodiments, R³ is —OR. In some embodiments, R³ is —N(R)₂. In some embodiments, R³ is —NO₂. In some embodiments, R³ is —N₃. In some embodiments, R³ is —SR. In some embodiments, R³ is —S(O)R. In some embodiments, R³ is —S(O)₂R. In some embodiments, R³ is —S(O)₂N(R)₂. In some embodiments, R³ is —NRS(O)₂R. In some embodiments, R³ is -L¹-.

In some embodiments, R³ is hydrogen. In some embodiments, R³ is an optionally substituted C₁₋₆ aliphatic group. In some embodiments, R³ is an optionally substituted 4-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, and sulfur. In some embodiments, R³ is an optionally substituted 5-6 membered monocyclic heteroaromatic ring having 1-4 heteroatoms independently selected from nitrogen, oxygen, and sulfur. In some embodiments, R³ is an optionally substituted 8-10 membered bicyclic heteroaromatic ring having 1-5 heteroatoms independently selected from nitrogen, oxygen, and sulfur.

In some embodiments, R³ is a C₁₋₈ bivalent straight or branched hydrocarbon chain. In some embodiments, R³ is a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R³ is selected from H, F, Cl, OH, Me, Et, i-Pr,

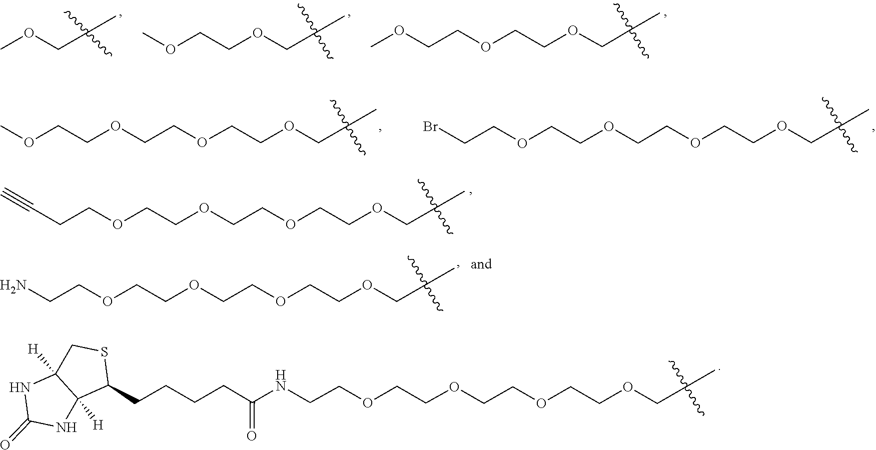

In some embodiments, R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² and R³ taken together with the carbons to which they are attached form a 4-8 membered saturated or partially unsaturated monocyclic carbocyclic ring. In some embodiments, R² and R³ taken together with the carbons to which they are attached form phenyl. In some embodiments, R² and R³ taken together with the carbons to which they are attached form an 8-10 membered bicyclic aromatic carbocyclic ring. In some embodiments, R² and R³ taken together with the carbons to which they are attached form a 4-8 membered saturated or partially unsaturated monocyclic heterocyclic ring having 1-2 heteroatoms independently selected from nitrogen, oxygen, and sulfur. In some embodiments, R² and R³ taken together with the carbons to which they are attached form a 5-6 membered monocyclic heteroaromatic ring having 1-4 heteroatoms independently selected from nitrogen, oxygen, and sulfur. In some embodiments, R² and R³ taken together with the carbons to which they are attached form an 8-10 membered bicyclic heteroaromatic ring having 1-5 heteroatoms independently selected from nitrogen, oxygen, and sulfur.

In some embodiments, R² and R³ taken together with the carbons to which they are attached form a cyclohexane ring. In some embodiments, R² and R³ taken together with the carbons to which they are attached form a cyclopentane ring.

In some embodiments, R² and R³ are selected from those depicted in Table 1, below.

As defined generally above, each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁴ is R. In some embodiments, R⁴ is halogen. In some embodiments, R⁴ is ═O. In some embodiments, R⁴ is —CN. In some embodiments, R⁴ is —NC. In some embodiments, R⁴ is —C(O)OR. In some embodiments, R⁴ is —OC(O)R. In some embodiments, R⁴ is —C(O)N(R)₂. In some embodiments, R⁴ is —N(R)C(O)R. In some embodiments, R⁴ is —N(R)C(O)N(R)₂. In some embodiments, R⁴ is —OC(O)N(R)₂. In some embodiments, R⁴ is —N(R)C(O)OR. In some embodiments, R⁴ is —OR. In some embodiments, R⁴ is —N(R)₂. In some embodiments, R⁴ is —NO₂. In some embodiments, R⁴ is —N₃. In some embodiments, R⁴ is —SR. In some embodiments, R⁴ is —S(O)R. In some embodiments, R⁴ is —S(O)₂R. In some embodiments, R⁴ is —S(O)₂N(R)₂. In some embodiments, R⁴ is —NRS(O)₂R.

In some embodiments, R⁴ is hydrogen. In some embodiments, R⁴ is an optionally substituted C₁₋₆ aliphatic group. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, and sulfur. In some embodiments, R⁴ is an optionally substituted 5-6 membered monocyclic heteroaromatic ring having 1-4 heteroatoms independently selected from nitrogen, oxygen, and sulfur. In some embodiments, R⁴ is an optionally substituted 8-10 membered bicyclic heteroaromatic ring having 1-5 heteroatoms independently selected from nitrogen, oxygen, and sulfur.

In some embodiments, R⁴ is selected from H, ═O, Me, Et, iPr,

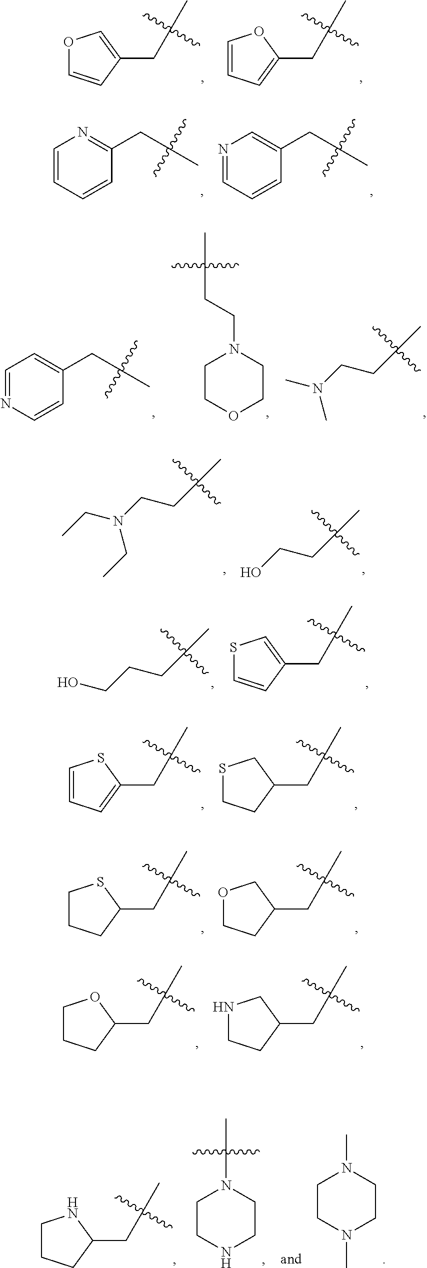

In some embodiments, R⁴ is selected from those depicted in Table 1, below.

As defined generally above, each R⁵ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁵ is R. In some embodiments, R⁵ is halogen. In some embodiments, R⁵ is —CN. In some embodiments, R⁵ is —NC. In some embodiments, R⁵ is —C(O)OR. In some embodiments, R⁵ is —OC(O)R. In some embodiments, R⁵ is —C(O)N(R)₂. In some embodiments, R⁵ is —N(R)C(O)R. In some embodiments, R⁵ is —N(R)C(O)N(R)₂. In some embodiments, R⁵ is —OC(O)N(R)₂. In some embodiments, R⁵ is —N(R)C(O)OR. In some embodiments, R⁵ is —OR. In some embodiments, R⁵ is —N(R)₂. In some embodiments, R⁵ is —NO₂. In some embodiments, R⁵ is —N₃. In some embodiments, R⁵ is —SR. In some embodiments, R⁵ is —S(O)R. In some embodiments, R⁵ is —S(O)₂R. In some embodiments, R⁵ is —S(O)₂N(R)₂. In some embodiments, R⁵ is —NRS(O)₂R.

In some embodiments, R⁵ is selected from those depicted in Table 1, below.

As defined generally above, -L²- is

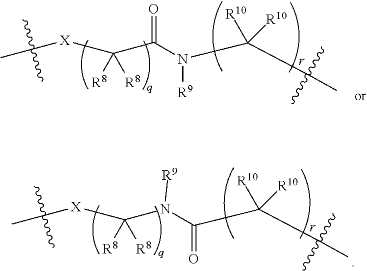

In some embodiments, -L²- is

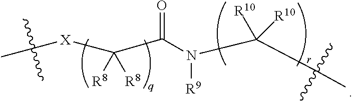

In some embodiments, -L²- is

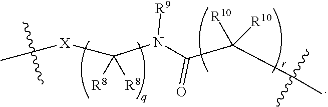

As defined generally above, X is NR⁶, O, CR⁶R⁷ or S.

As defined generally above, each R⁶ and R⁷ is independently —R, halogen, —CN, —NC, —C(O)OR, —OC(O)R, —OR, —N(R)₂, or —SR.

In some embodiments, R⁶ is R. In some embodiments, R⁶ is halogen. In some embodiments, R⁶ is —CN. In some embodiments, R⁶ is —NC. In some embodiments, R⁶ is —C(O)OR. In some embodiments, R⁶ is —OC(O)R. In some embodiments, R⁶ is —OR. In some embodiments, R⁶ is —N(R)₂. In some embodiments, R⁶ is —SR.

In some embodiments, R⁶ is hydrogen. In some embodiments, R⁶ is an optionally substituted C₁₋₆ aliphatic group. In some embodiments, R⁶ is an optionally substituted 3-8 membered saturated or partially unsaturated monocyclic carbocyclic ring. In some embodiments, RP is an optionally substituted phenyl. In some embodiments, R⁶ is an optionally substituted 8-10 membered bicyclic aromatic carbocyclic ring. In some embodiments, R⁶ is an optionally substituted 4-8 membered saturated or partially unsaturated monocyclic heterocyclic ring having 1-2 heteroatoms independently selected from nitrogen, oxygen, and sulfur. In some embodiments, R⁶ is an optionally substituted 5-6 membered monocyclic heteroaromatic ring having 1-4 heteroatoms independently selected from nitrogen, oxygen, and sulfur. In some embodiments, R⁶ is an optionally substituted 8-10 membered bicyclic heteroaromatic ring having 1-5 heteroatoms independently selected from nitrogen, oxygen, and sulfur.

In some embodiments, R⁶ is selected from H, Me, Et, iPr and OH.

In some embodiments, R⁶ is selected from those depicted in Table 1, below.

In some embodiments, R⁷ is R. In some embodiments, R⁷ is halogen. In some embodiments, R⁷ is —CN. In some embodiments, R⁷ is —NC. In some embodiments, R⁷ is —C(O)OR. In some embodiments, R⁷ is —OC(O)R. In some embodiments, R⁷ is —OR. In some embodiments, R⁷ is —N(R)₂. In some embodiments, R⁷ is —SR.

In some embodiments, R⁷ is hydrogen. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, and sulfur. In some embodiments, R⁷ is an optionally substituted 5-6 membered monocyclic heteroaromatic ring having 1-4 heteroatoms independently selected from nitrogen, oxygen, and sulfur. In some embodiments, R⁷ is an optionally substituted 8-10 membered bicyclic heteroaromatic ring having 1-5 heteroatoms independently selected from nitrogen, oxygen, and sulfur.

In some embodiments, R⁷ is selected from H, Me, Et, iPr and OH.

In some embodiments, R⁷ is selected from those depicted in Table 1, below.

In some embodiments, X is selected from NH, O, S, NMe, CHMe, CHOH, C(Me)₂ and C(Me)OH.

In some embodiments, X is selected from those depicted in Table 1, below.

As defined generally above, each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or -L¹-.

In some embodiments, R⁸ is R. In some embodiments, R⁸ is halogen. In some embodiments, R⁸ is —CN. In some embodiments, R⁸ is —NC. In some embodiments, R⁸ is —C(O)OR. In some embodiments, R⁸ is —OC(O)R. In some embodiments, R⁸ is —C(O)N(R)₂. In some embodiments, R⁸ is —N(R)C(O)R. In some embodiments, R⁸ is —N(R)C(O)N(R)₂. In some embodiments, R⁸ is —OC(O)N(R)₂. In some embodiments, R⁸ is —N(R)C(O)OR. In some embodiments, R⁸ is —OR. In some embodiments, R⁸ is —N(R)₂. In some embodiments, R⁸ is —NO₂. In some embodiments, R⁸ is —N₃. In some embodiments, R⁸ is —SR. In some embodiments, R⁸ is —S(O)R. In some embodiments, R⁸ is —S(O)₂R. In some embodiments, R⁸ is —S(O)₂N(R)₂. In some embodiments, R⁸ is —NRS(O)₂R. In some embodiments, R⁸ is -L¹-.

In some embodiments, R⁸ is hydrogen. In some embodiments, R⁸ is an optionally substituted C₁₋₆ aliphatic group. In some embodiments, R⁸ is an optionally substituted 3-8 membered saturated or partially unsaturated monocyclic carbocyclic ring. In some embodiments, R⁸ is an optionally substituted phenyl. In some embodiments, R⁸ is an optionally substituted 8-10 membered bicyclic aromatic carbocyclic ring. In some embodiments, R⁸ is an optionally substituted 4-8 membered saturated or partially unsaturated monocyclic heterocyclic ring having 1-2 heteroatoms independently selected from nitrogen, oxygen, and sulfur. In some embodiments, R⁸ is an optionally substituted 5-6 membered monocyclic heteroaromatic ring having 1-4 heteroatoms independently selected from nitrogen, oxygen, and sulfur. In some embodiments, R⁸ is an optionally substituted 8-10 membered bicyclic heteroaromatic ring having 1-5 heteroatoms independently selected from nitrogen, oxygen, and sulfur.

In some embodiments, R⁸ is a C₁₋₈ bivalent straight or branched hydrocarbon chain. In some embodiments, R⁸ is a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, one instance of —C(R⁸)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring. In some embodiments, one instance of —C(R⁸)₂— is optionally replaced by phenyl. In some embodiments, one instance of —C(R⁸)₂— is optionally replaced by an 8-10 membered bicyclic aromatic carbocyclic ring. In some embodiments, one instance of —C(R⁸)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R⁸)₂— is optionally replaced by a 5-6 membered monocyclic heteroaromatic ring having 1-4 heteroatoms independently selected from nitrogen, oxygen, and sulfur. In some embodiments, one instance of —C(R⁸)₂— is optionally replaced by an 8-10 membered bicyclic heteroaromatic ring having 1-5 heteroatoms independently selected from nitrogen, oxygen, and sulfur.

In some embodiments, one instance of —C(R⁸)₂— is optionally replaced by

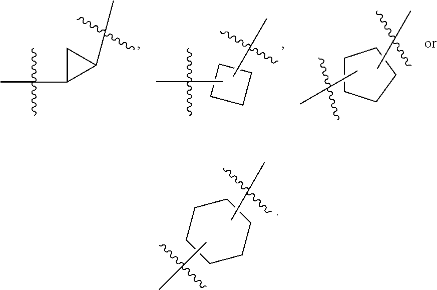

In some embodiments, R⁸ is selected from H, F, Cl, OH, Me, Et, i-Pr

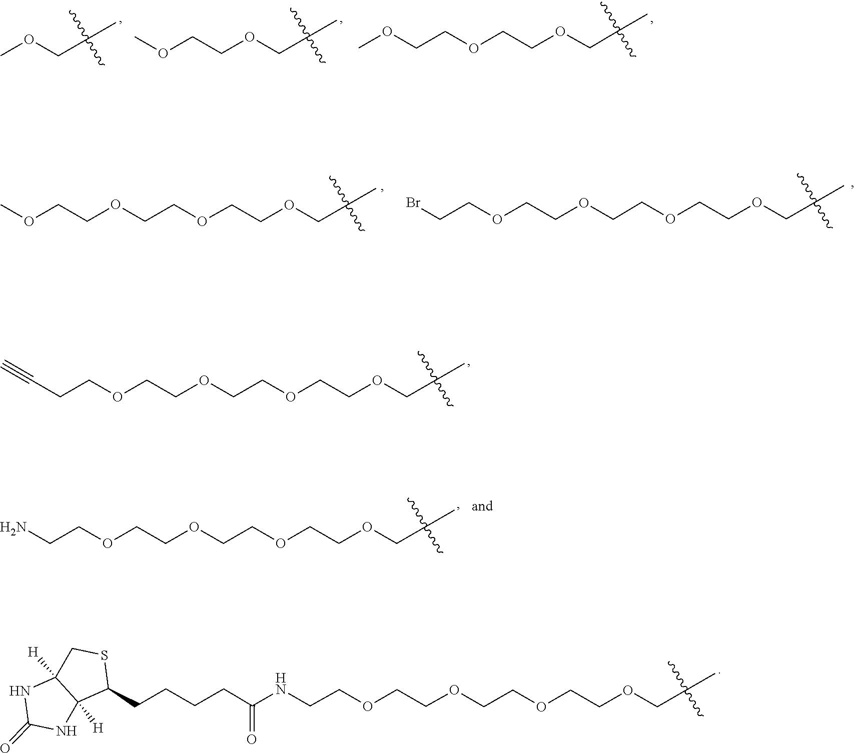

In some embodiments, R⁸ is selected from those depicted in Table 1, below.

As defined generally above, R⁹ is —R, halogen, —CN, —NC, —C(O)OR, —OC(O)R, —OR, —N(R)₂, or —SR.

In some embodiments, R⁹ is R. In some embodiments, R⁹ is halogen. In some embodiments, R⁹ is —CN. In some embodiments, R⁹ is —NC. In some embodiments, R⁹ is —C(O)OR. In some embodiments, R⁹ is —OC(O)R. In some embodiments, R⁹ is —OR. In some embodiments, R⁹ is —N(R)₂. In some embodiments, R⁹ is —SR.

In some embodiments, R⁹ is hydrogen. In some embodiments, R⁹ is an optionally substituted C₁₋₆ aliphatic group. In some embodiments, R⁹ is an optionally substituted 3-8 membered saturated or partially unsaturated monocyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 4-8 membered saturated or partially unsaturated monocyclic heterocyclic ring having 1-2 heteroatoms independently selected from nitrogen, oxygen, and sulfur. In some embodiments, R⁹ is an optionally substituted 5-6 membered monocyclic heteroaromatic ring having 1-4 heteroatoms independently selected from nitrogen, oxygen, and sulfur. In some embodiments, R⁹ is an optionally substituted 8-10 membered bicyclic heteroaromatic ring having 1-5 heteroatoms independently selected from nitrogen, oxygen, and sulfur.

In some embodiments, R⁹ is selected from H, Me, Et, iPr and OH.

In some embodiments, R⁹ is selected from those depicted in Table 1, below.

In some embodiments, R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur.

As defined generally above, R¹⁰ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹⁰ is R. In some embodiments, R¹⁰ is halogen. In some embodiments, R¹⁰ is —CN. In some embodiments, R¹⁰ is —NC. In some embodiments, R¹⁰ is —C(O)OR. In some embodiments, R¹⁰ is —OC(O)R. In some embodiments, R¹⁰ is —C(O)N(R)₂. In some embodiments, R¹⁰ is —N(R)C(O)R. In some embodiments, R¹⁰ is —N(R)C(O)N(R)₂. In some embodiments, R¹⁰ is —OC(O)N(R)₂. In some embodiments, R¹⁰ is —N(R)C(O)OR. In some embodiments, R¹⁰ is —OR. In some embodiments, R¹⁰ is —N(R)₂. In some embodiments, R¹⁰ is —NO₂. In some embodiments, R¹⁰ is —N₃. In some embodiments, R¹⁰ is —SR. In some embodiments, R¹⁰ is —S(O)R. In some embodiments, R¹⁰ is —S(O)₂R. In some embodiments, R¹⁰ is —S(O)₂N(R)₂. In some embodiments, R¹⁰ is —NRS(O)₂R.

In some embodiments, R¹⁰ is hydrogen. In some embodiments, R¹⁰ is an optionally substituted C₁₋₆ aliphatic group. In some embodiments, R¹⁰ is an optionally substituted 3-8 membered saturated or partially unsaturated monocyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 4-8 membered saturated or partially unsaturated monocyclic heterocyclic ring having 1-2 heteroatoms independently selected from nitrogen, oxygen, and sulfur. In some embodiments, R¹⁰ is an optionally substituted 5-6 membered monocyclic heteroaromatic ring having 1-4 heteroatoms independently selected from nitrogen, oxygen, and sulfur. In some embodiments, R¹⁰ is an optionally substituted 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring. In some embodiments, one instance of —C(R¹⁰)₂— is optionally replaced by phenyl. In some embodiments, one instance of —C(R¹⁰)₂— is optionally replaced by an 8-10 membered bicyclic aromatic carbocyclic ring. In some embodiments, one instance of —C(R¹⁰)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R¹⁰)₂— is optionally replaced by a 5-6 membered monocyclic heteroaromatic ring having 1-4 heteroatoms independently selected from nitrogen, oxygen, and sulfur. In some embodiments, one instance of —C(R¹⁰)₂— is optionally replaced by an 8-10 membered bicyclic heteroaromatic ring having 1-5 heteroatoms independently selected from nitrogen, oxygen, and sulfur.

In some embodiments, one instance of —C(R¹⁰)₂— is optionally replaced by

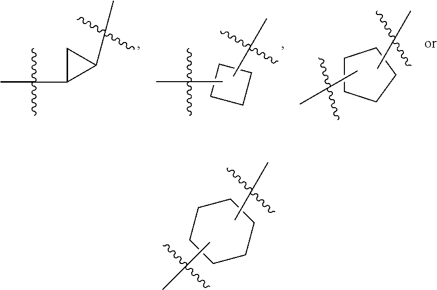

In some embodiments, R¹⁰ is selected from H, Me, Et, iPr and CH₃OH.

In some embodiments, R¹⁰ is selected from those depicted in Table 1, below.

In some embodiments, R⁸ and R¹⁰ taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur.

In some embodiments, R⁹ and R¹⁰ taken together with the atoms to which they are attached, form a ring substituted with m instances of R⁵; wherein the ring is a 4-8 membered saturated or partially unsaturated monocyclic heterocyclic ring having 1-2 heteroatoms independently selected from nitrogen, oxygen, and sulfur.

In some embodiments, -L²- is selected from

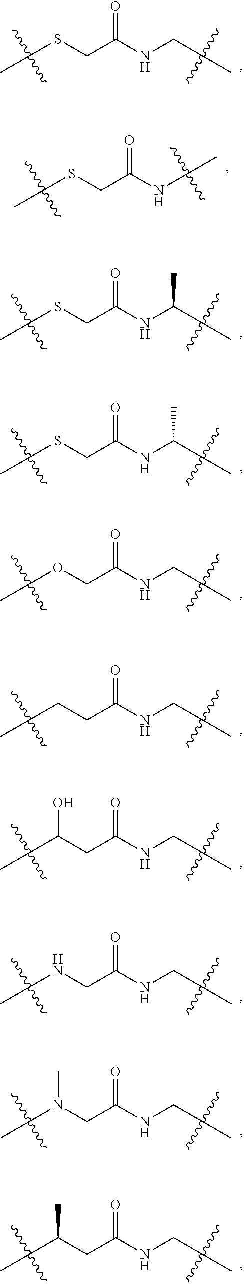

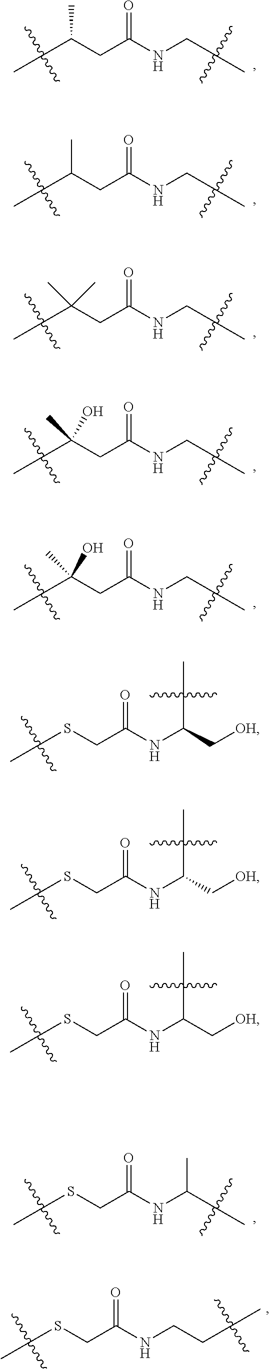

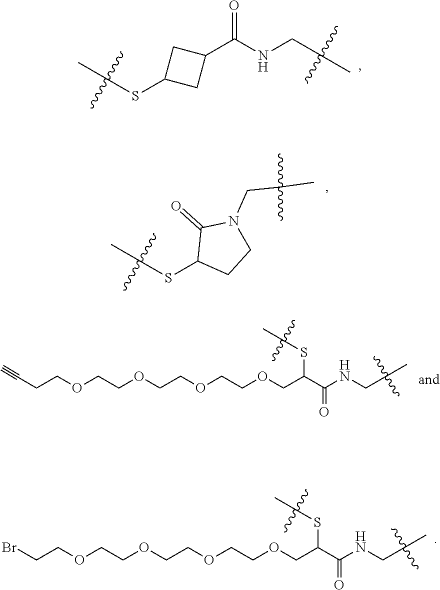 [SMILES: *CNC(=O)C1CC(S*)C1 ; *CN1CCC(S*)C1=O ; *CNC(=O)C(COCCOCCOCCOCCC#C)S* ; *CNC(=O)C(COCCOCCOCCOCCBr)S*]

In some embodiments, -L²- is selected from those depicted in Table 1, below.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0, 1, 2, or 3. In some embodiments, m is 0, 1, or 2. In some embodiments, m is 1, 2, or 3.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0, 1, 2, or 3. In some embodiments, n is 0, 1, or 2. In some embodiments, n is 1, 2, or 3.

As defined generally above, p is 0, 1, 2, or 3. In some embodiments, p is 0. In some embodiments, p is 1. In some embodiments, p is 2. In some embodiments, p is 3. In some embodiments, p is 0, 1, 2, or 3. In some embodiments, p is 0, 1, or 2. In some embodiments, p is 1, 2, or 3.

As defined generally above, q is 0, 1, 2, 3, or 4. In some embodiments, q is 0. In some embodiments, q is 1. In some embodiments, q is 2. In some embodiments, q is 3. In some embodiments, q is 4. In some embodiments, q is 0, 1, 2, or 3. In some embodiments, q is 0, 1, or 2. In some embodiments, q is 1, 2, or 3.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0, 1, 2, or 3. In some embodiments, r is 0, 1, or 2. In some embodiments, r is 1, 2, or 3.

Exemplary

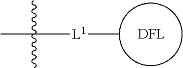

compounds of the invention are set forth in Table 1 and Table 1A, below.

Exemplary

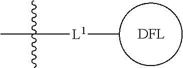

compounds of the invention are set forth in Table 1, Table 1A, and Table 1B below.

Exemplary

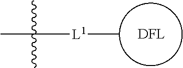

compounds of the invention are set forth in Table 1, Table 1A, Table 1B, and Table 1C below.

In some embodiments,

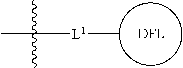

is a compound of Formula I-c:

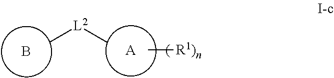

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur;
Ring B is

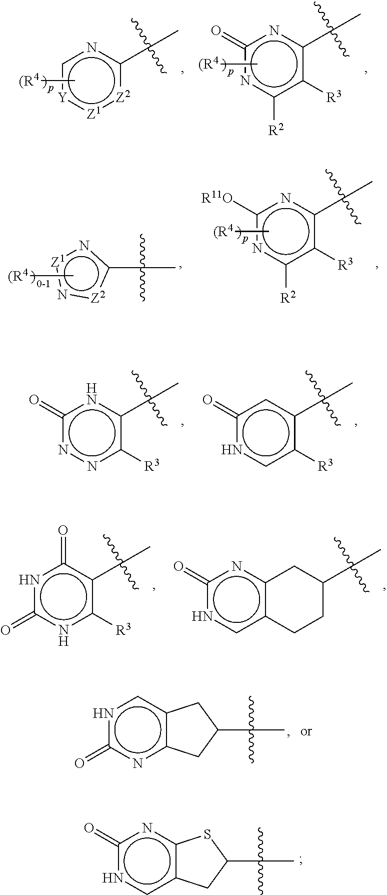

Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is

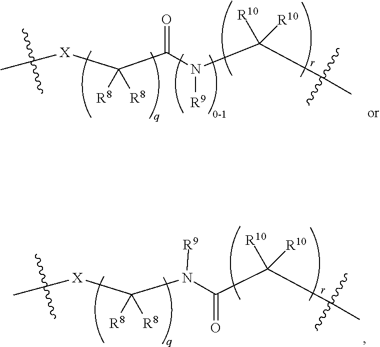

wherein —X— is covalently bound to Ring B; —X— is a bond, —NR⁶, —O—, —CR⁶R⁷—, —C(O)—, —S—, or —S(O)₂—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or two R⁸, taken together with the carbon atom to which they are attached, form a 3-6 membered carbocyclic ring;
R⁹ is —R, halogen, —CN, —NC, —C(O)OR, —OC(O)R, —OR, —N(R)₂, or —SR; or R⁸ and R⁹, taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur;
each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or two R¹⁰, taken together with the carbon atom to which they are attached, form a 3-6 membered carbocyclic ring; or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a bivalent linker group that covalently connects the RNA Binder to the DFL; wherein one and only one of R¹, R², R³, R⁸, or R¹¹ is -L¹- and one end of -L¹- is covalently bound to the RNA Binder;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
R¹¹ is H, C₁₋₃ alkyl, or -L¹-;
m is 0, 1, 2, 3, or 4;
n is 0, 1, 2, 3, 4, or 5;
p is 0, 1, 2, or 3;
q is 0, 1, 2, 3, or 4; and
r is 0, 1, 2, 3, or 4.

As defined generally above, Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur.

In some embodiments, Ring A is a 3-8 membered saturated or partially unsaturated monocyclic carbocyclic ring. In some embodiments, Ring A is phenyl. In some embodiments, Ring A is an 8-10 membered bicyclic aromatic carbocyclic ring. In some embodiments, Ring A is a 4-8 membered saturated or partially unsaturated monocyclic heterocyclic ring having 1-3 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is an 8-10 membered bicyclic heteroaromatic ring having 1-5 heteroatoms independently selected from nitrogen, oxygen, and sulfur. In some embodiments, Ring A is an 8-12 membered bicyclic heterocyclic ring having 1-5 heteroatoms independently selected from nitrogen, oxygen, and sulfur. In some embodiments, Ring A is an 8-12 membered tricyclic heterocyclic ring having 1-5 heteroatoms independently selected from nitrogen, oxygen, and sulfur.

In some embodiments, Ring A is selected from:

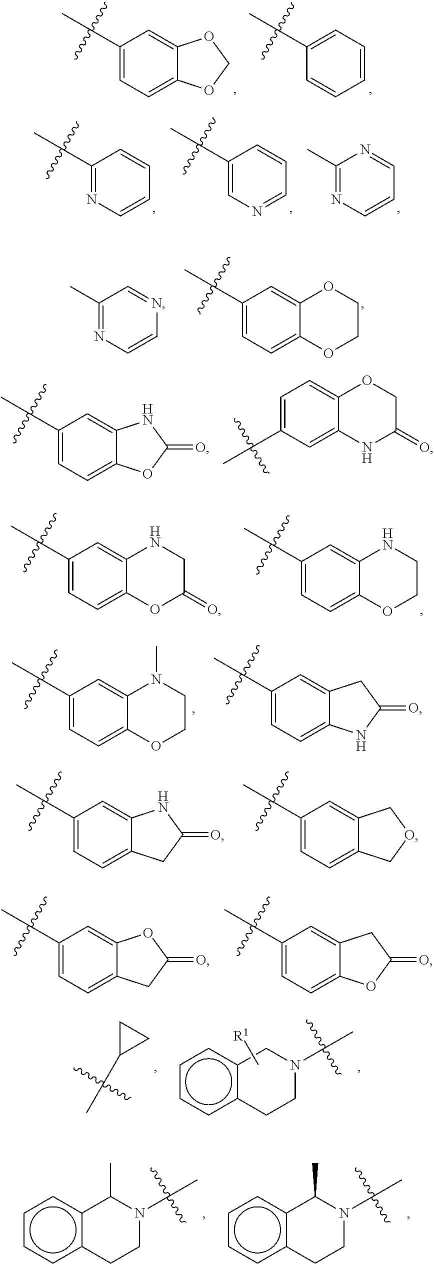

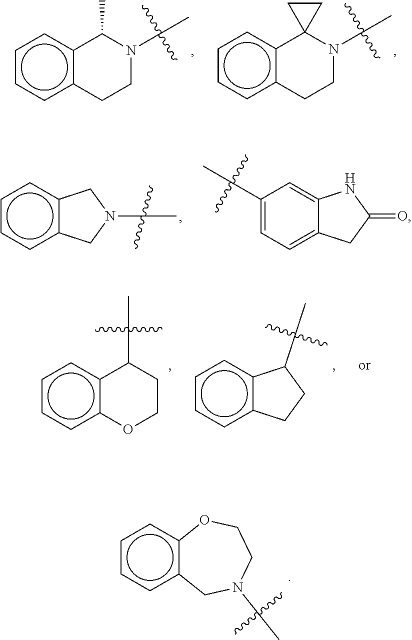

In some embodiments, Ring A is selected from

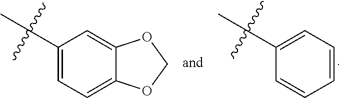 [SMILES: *c1ccc2c(c1)OCO2 ; *c1ccccc1]

In some embodiments, Ring A is selected from those depicted in Table 1, below.

In some embodiments, Ring A is selected from those depicted in Table 1A, below.

In some embodiments, Ring A is selected from those depicted in Table 1B, below.

In some embodiments, Ring A is selected from those depicted in Table 1C, below.

As defined generally above, Ring B is

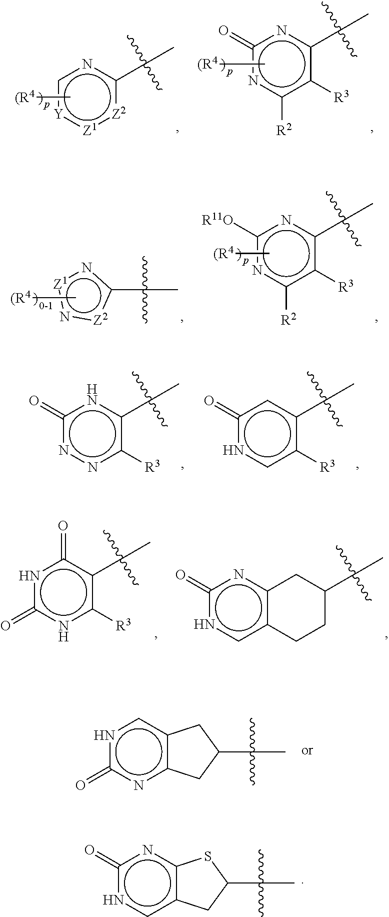

In some embodiments, Ring B is

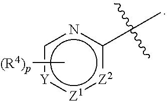

In some embodiments, Ring B is

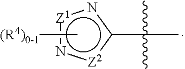

In some embodiments, Ring B is

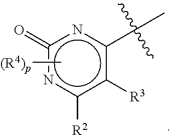

In some embodiments, Ring B is

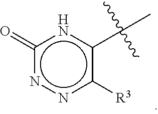

In some embodiments, Ring B is

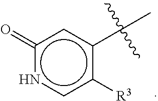

In some embodiments, Ring B is

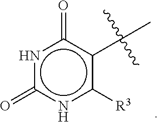

In some embodiments, Ring B is

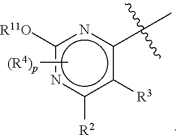

In some embodiments, Ring B is

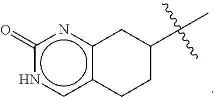

In some embodiments, Ring B is

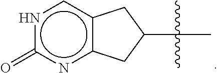

In some embodiments, Ring B is

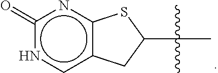

In some embodiments, Ring B is

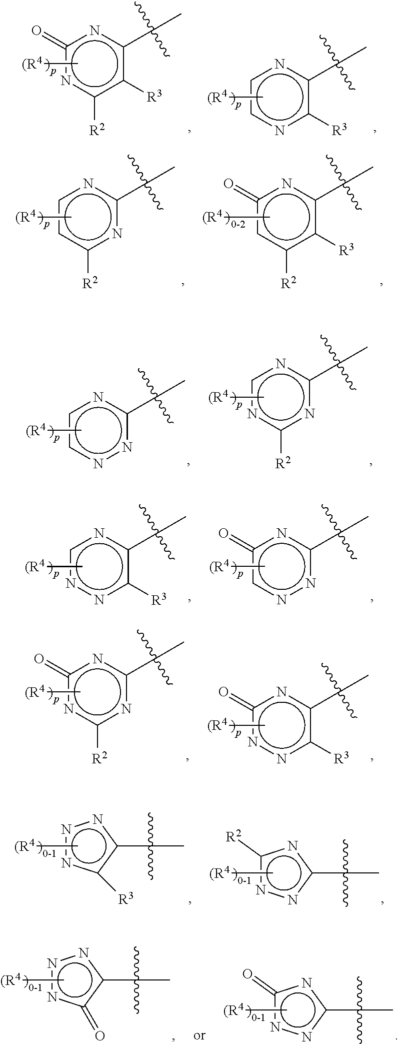

In some embodiments, Ring B is selected from

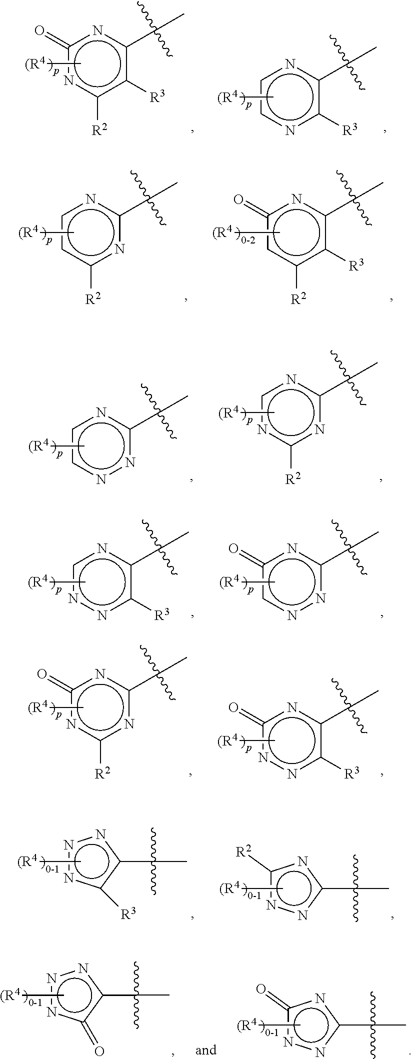

In some embodiments, Ring B is

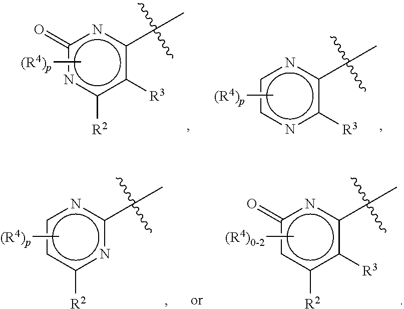

In some embodiments, Ring B is selected from

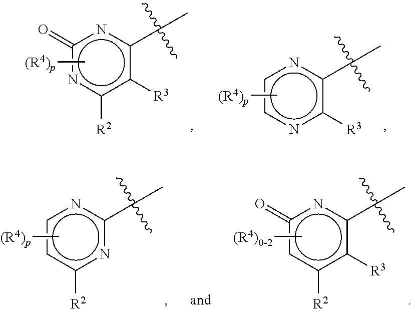

In some embodiments, Ring B is

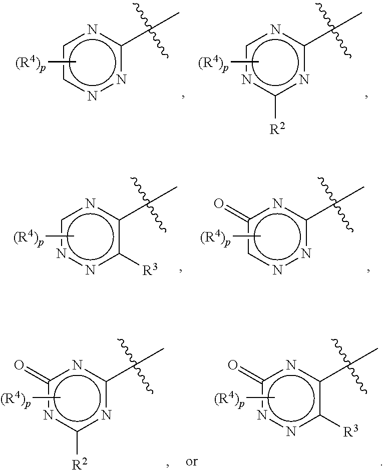

In some embodiments, Ring B is selected from

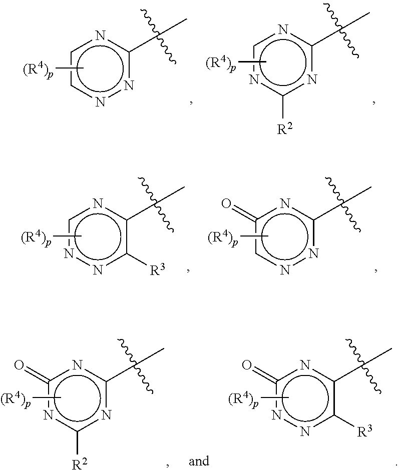

In some embodiments, Ring B is

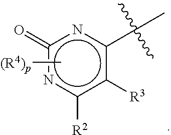

In some embodiments, Ring B is

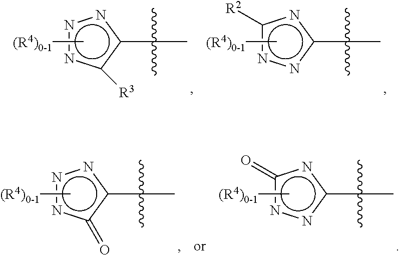

In some embodiments, Ring B is selected from

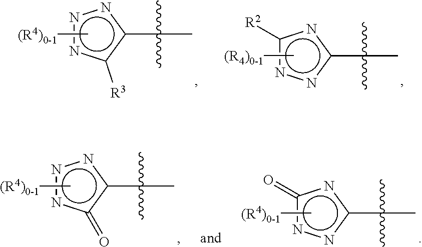

In some embodiments, Ring B is selected from those depicted in Table 1, below.

In some embodiments, Ring B is selected from those depicted in Table 1A, below.

In some embodiments, Ring B is selected from those depicted in Table 1B, below.

In some embodiments, Ring B is selected from those depicted in Table 1C, below.

As defined generally above, Y is N or CH.

In some embodiments, Y is N. In some embodiments, Y is CH.

In some embodiments, Y is CH. In some embodiments, Y is substituted with R⁴.

In some embodiments, Y is selected from those depicted in Table 1, below.

In some embodiments, Y is selected from those depicted in Table 1A, below.

In some embodiments, Y is selected from those depicted in Table 1B, below.

In some embodiments, Y is selected from those depicted in Table 1C, below.

As defined generally above, Z¹ is N, C═O, or CR².

In some embodiments, Z¹ is N. In some embodiments, Z¹ is C═O. In some embodiments, Z¹ is CR².

In some embodiments, Z¹ is selected from those depicted in Table 1, below.

In some embodiments, Z¹ is selected from those depicted in Table 1A, below.

In some embodiments, Z¹ is selected from those depicted in Table 1B, below.

In some embodiments, Z¹ is selected from those depicted in Table 1C, below.

As defined generally above, Z² is N, C═O, or CR³.

In some embodiments, Z² is N. In some embodiments, Z² is C═O. In some embodiments, Z² is CR³.

In some embodiments, Z² is selected from those depicted in Table 1, below.

In some embodiments, Z² is selected from those depicted in Table 1A, below.

In some embodiments, Z² is selected from those depicted in Table 1B, below.

In some embodiments, Z² is selected from those depicted in Table 1C, below.

As defined generally above, each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-.

In some embodiments, R¹ is R. In some embodiments, R¹ is halogen. In some embodiments, R¹ is —CN. In some embodiments, R¹ is —NC. In some embodiments, R¹ is —C(O)OR. In some embodiments, R¹ is —OC(O)R. In some embodiments, R¹ is —C(O)N(R)₂. In some embodiments, R¹ is —N(R)C(O)R. In some embodiments, R¹ is —N(R)C(O)N(R)₂. In some embodiments, R¹ is —OC(O)N(R)₂. In some embodiments, R¹ is —N(R)C(O)OR. In some embodiments, R¹ is —OR. In some embodiments, R¹ is —N(R)₂. In some embodiments, R¹ is —NO₂. In some embodiments, R¹ is —N₃. In some embodiments, R¹ is —SR. In some embodiments, R¹ is —S(O)R. In some embodiments, R¹ is —S(O)₂R. In some embodiments, R¹ is —S(O)₂N(R)₂. In some embodiments, R¹ is —NRS(O)₂R. In some embodiments, R¹ is -L¹-.

In some embodiments, R¹ is hydrogen. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, and sulfur. In some embodiments, R¹ is an optionally substituted 5-6 membered monocyclic heteroaromatic ring having 1-4 heteroatoms independently selected from nitrogen, oxygen, and sulfur. In some embodiments, R¹ is an optionally substituted 8-10 membered bicyclic heteroaromatic ring having 1-5 heteroatoms independently selected from nitrogen, oxygen, and sulfur.

In some embodiments, R¹ is a C₁₋₈ bivalent straight or branched hydrocarbon chain. In some embodiments, R¹ is a C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R¹ is a C₁₋₁₆ bivalent straight or branched hydrocarbon chain. In some embodiments, R¹ is a C₁₋₁₆ bivalent straight or branched hydrocarbon chain wherein 1, 2, 3, 4, 5, or 6 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R¹ is selected from H, F, Cl, —CN, OH, OMe, Me, Et, i-Pr,

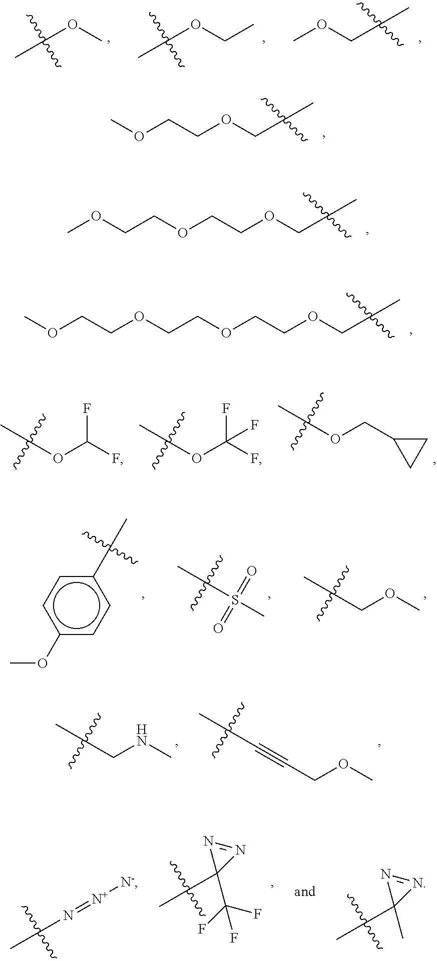

In some embodiments, R¹ is selected from H, F, Cl, —CN, OH, OMe, Me, Et, i-Pr

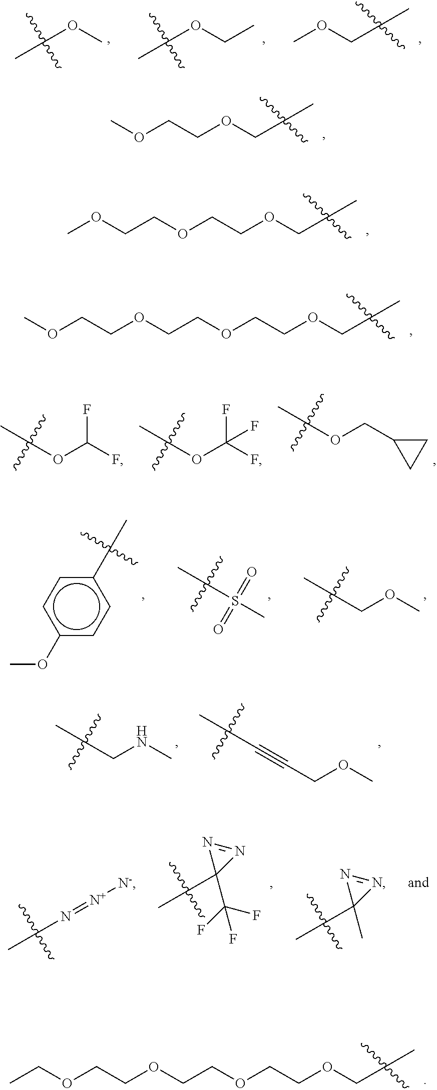

In some embodiments, R¹ is selected from H, F, Cl, —CN, OH, OMe, Me, Et, i-Pr

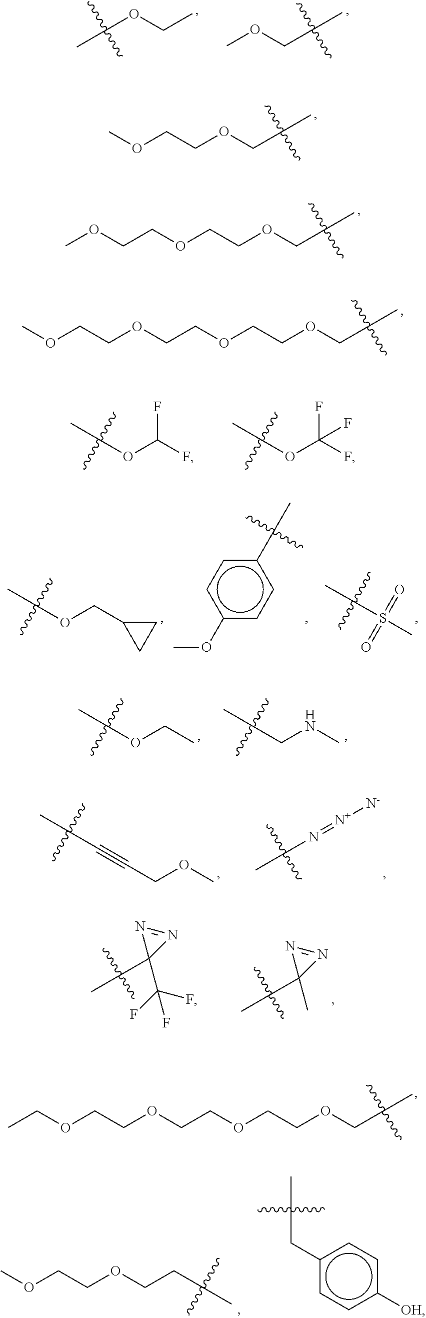

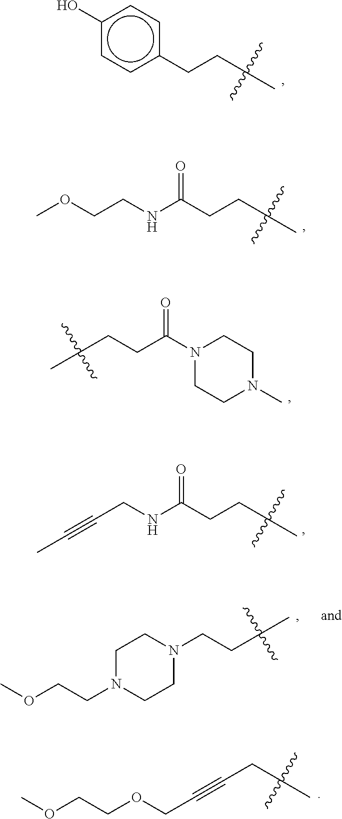

In some embodiments, R¹ is -L¹- and is selected from

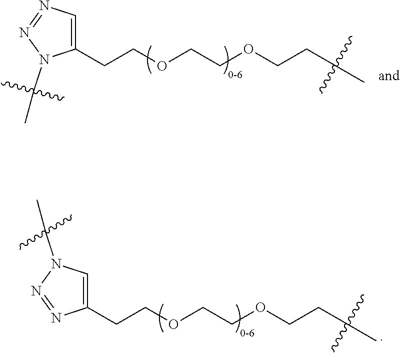

In some embodiments, R¹ is -L¹- and is selected from

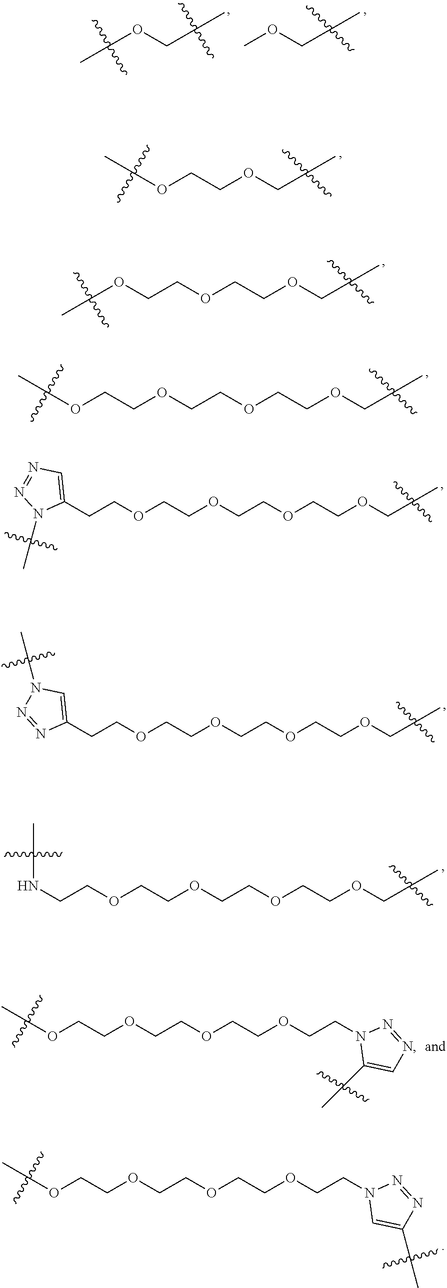

In some embodiments, R¹ is -L¹- and is selected from

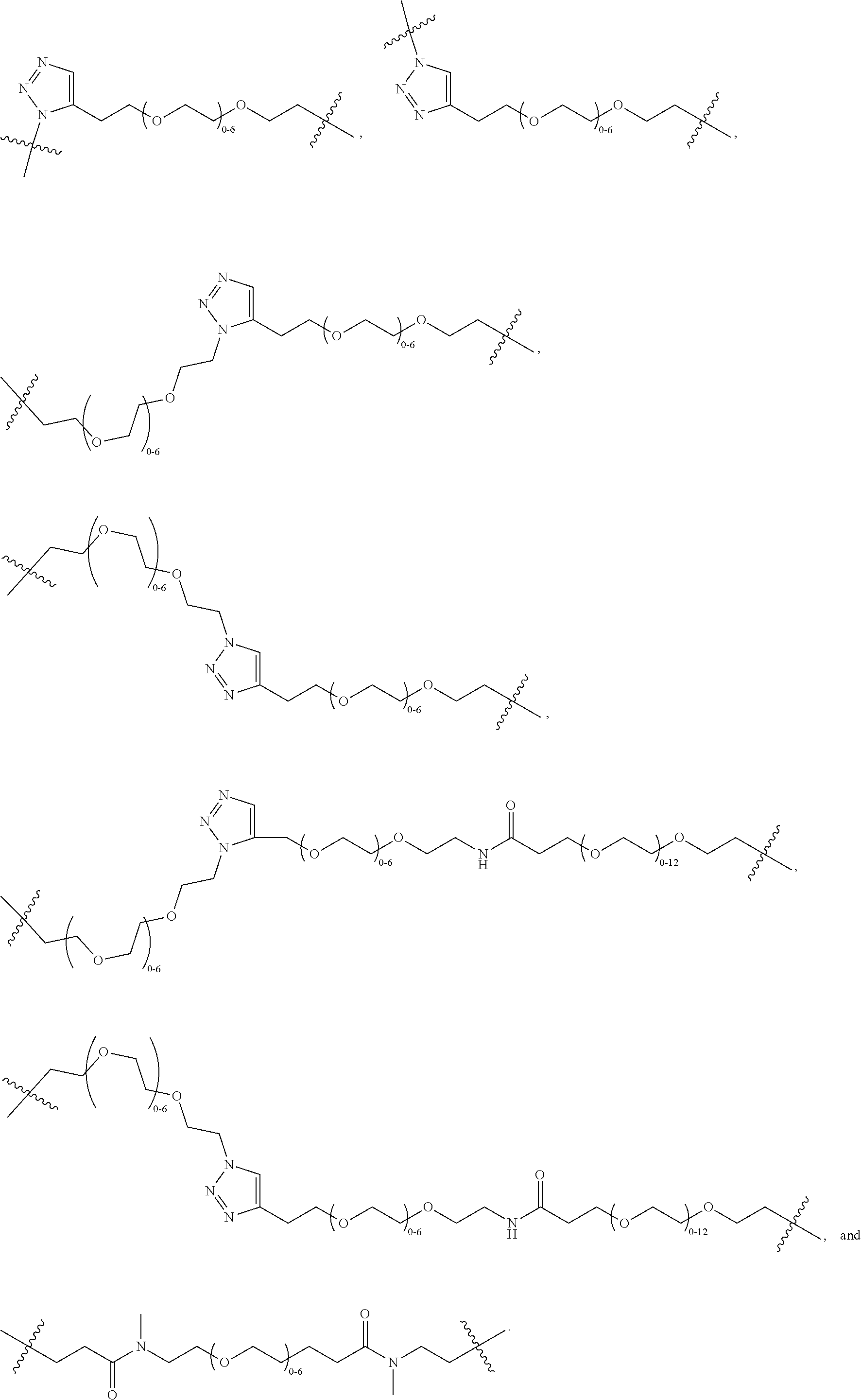

In some embodiments, R¹ is -L¹- and is selected from

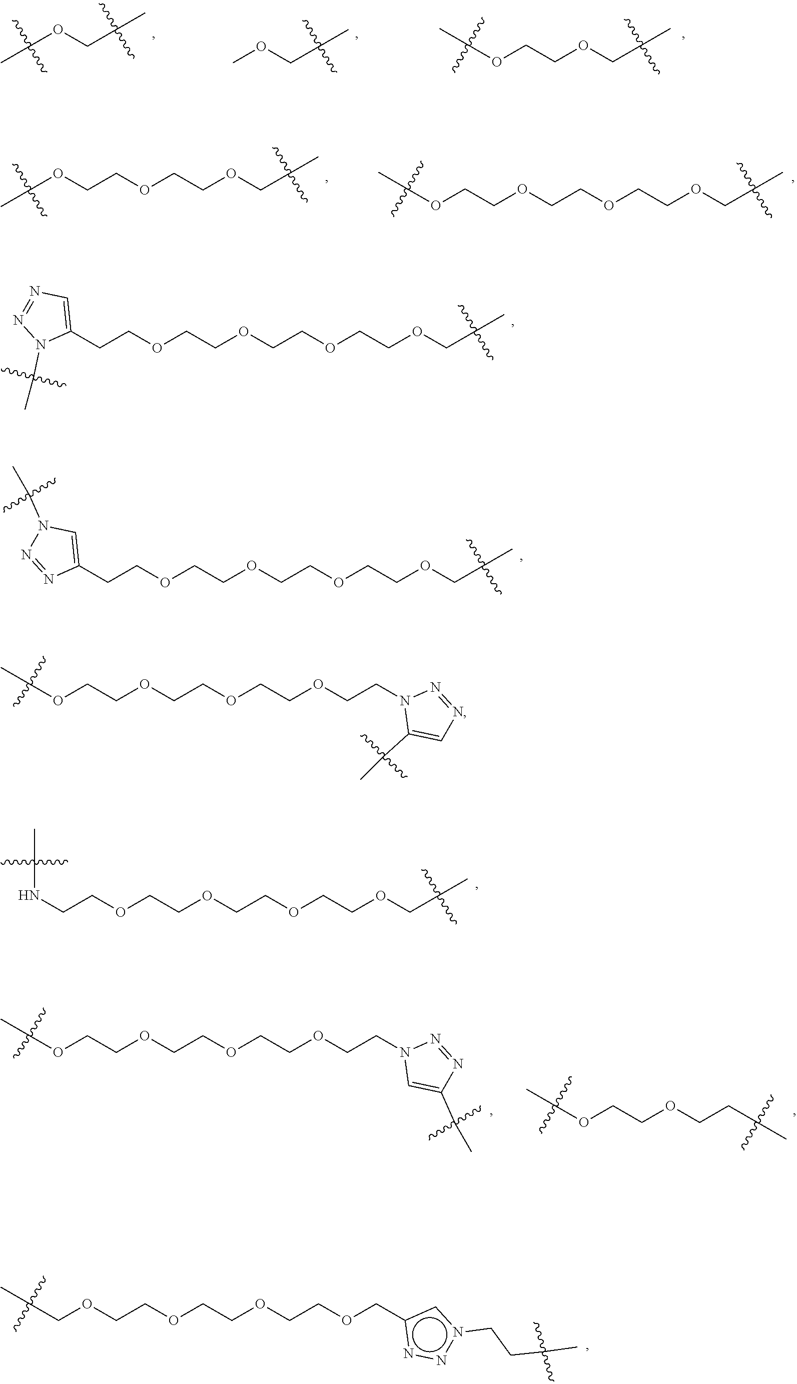

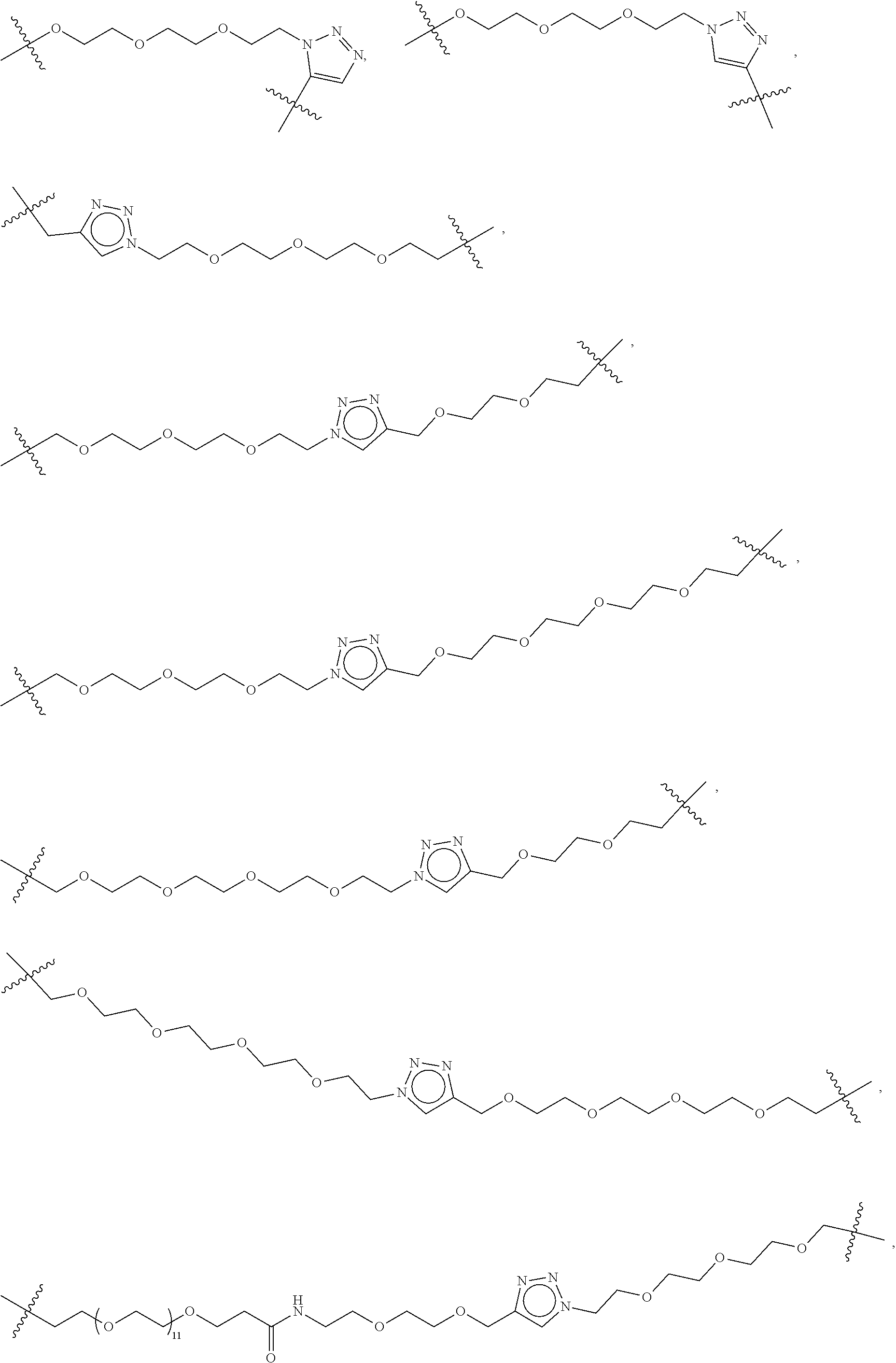

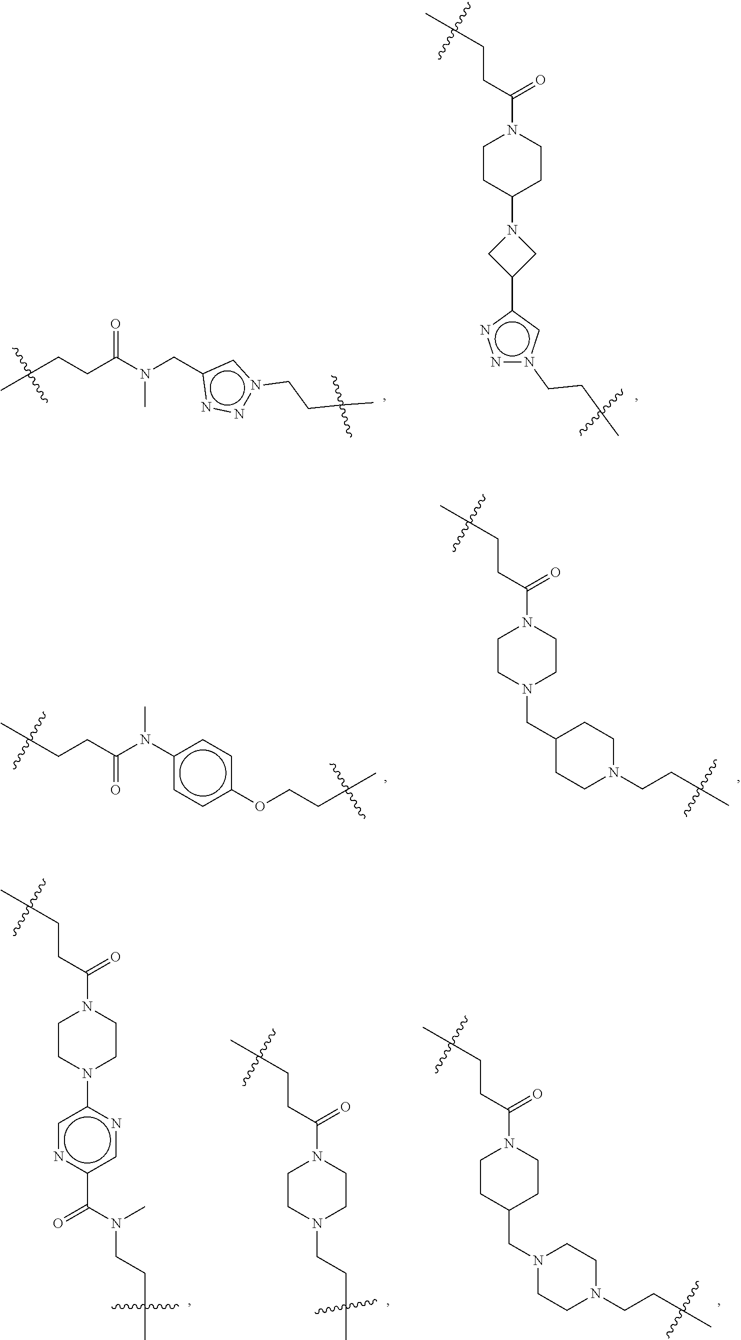

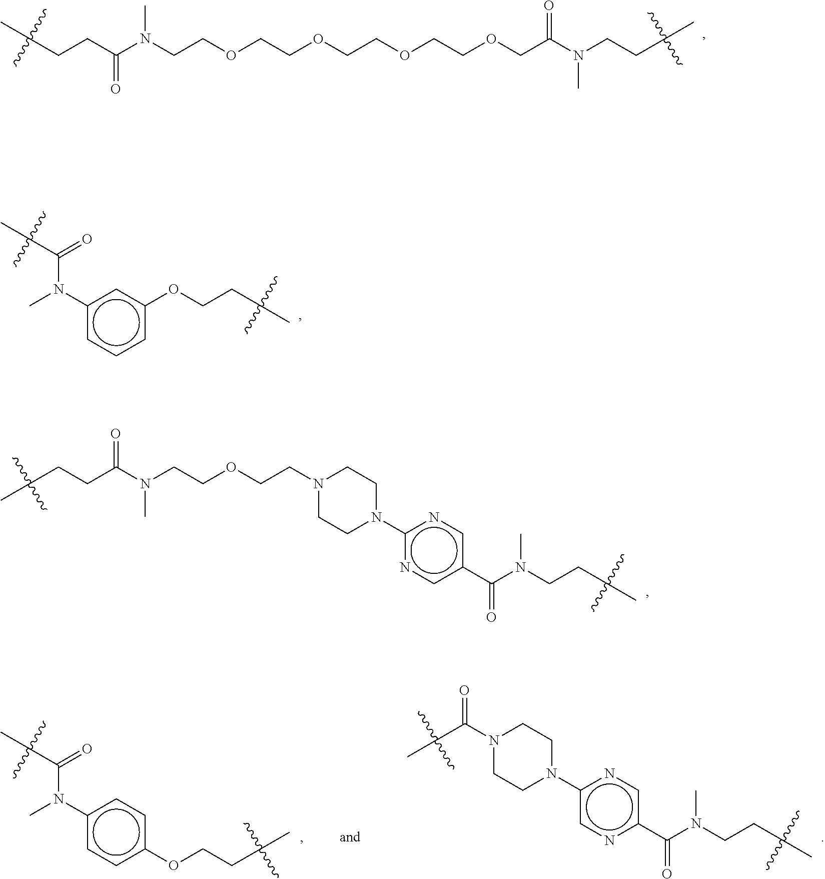

In some embodiments, R¹ is -L¹- and is selected from

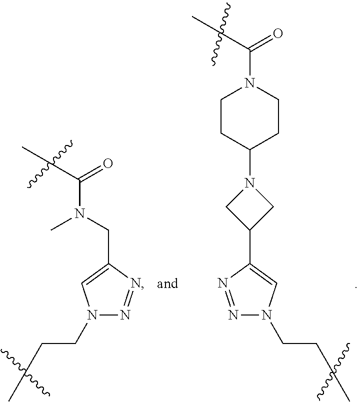 [SMILES: *CCn1cc(CN(C)C(*)=O)nn1 ; *CCn1cc(C2CN(C3CCN(C(*)=O)CC3)C2)nn1]

In some embodiments, R¹ is -L¹- and is selected from one of those in Table 3, below.

In some embodiments, R is selected from those depicted in Table 1, below.

In some embodiments, R¹ is selected from those depicted in Table 1A, below.

In some embodiments, R¹ is selected from those depicted in Table 1B, below.

In some embodiments, R¹ is selected from those depicted in Table 1C, below.

As defined generally above, each R² and R³ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² is R. In some embodiments, R² is halogen. In some embodiments, R² is —CN. In some embodiments, R² is —NC. In some embodiments, R² is —C(O)OR. In some embodiments, R² is —OC(O)R. In some embodiments, R² is —C(O)N(R)₂. In some embodiments, R² is —N(R)C(O)R. In some embodiments, R² is —N(R)C(O)N(R)₂. In some embodiments, R² is —OC(O)N(R)₂. In some embodiments, R² is —N(R)C(O)OR. In some embodiments, R² is —OR. In some embodiments, R² is —N(R)₂. In some embodiments, R² is —NO₂. In some embodiments, R² is —N₃. In some embodiments, R² is —SR. In some embodiments, R² is —S(O)R. In some embodiments, R² is —S(O)₂R. In some embodiments, R² is —S(O)₂N(R)₂. In some embodiments, R² is —NRS(O)₂R. In some embodiments, R² is -L¹-.

In some embodiments, R² is hydrogen. In some embodiments, R² is an optionally substituted C₁₋₆ aliphatic group. In some embodiments, R² is an optionally substituted 4-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, and sulfur. In some embodiments, R² is an optionally substituted 5-6 membered monocyclic heteroaromatic ring having 1-4 heteroatoms independently selected from nitrogen, oxygen, and sulfur. In some embodiments, R² is an optionally substituted 8-10 membered bicyclic heteroaromatic ring having 1-5 heteroatoms independently selected from nitrogen, oxygen, and sulfur.

In some embodiments, R² is a C₁₋₈ bivalent straight or branched hydrocarbon chain. In some embodiments, R² is a C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R² is a C₁₋₁₆ bivalent straight or branched hydrocarbon chain. In some embodiments, R² is a C₁₋₁₆ bivalent straight or branched hydrocarbon chain wherein 1, 2, 3, 4, 5 or 6 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R² is selected from H, F, Cl, OH, OMe, Me, Et, i-Pr,

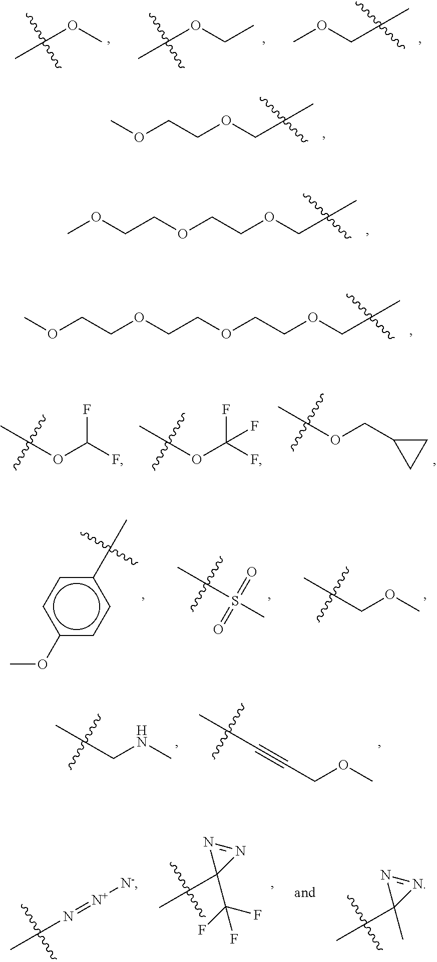

In some embodiments, R² is selected from H, F, Cl, OH, OMe, Me, Et, i-Pr,

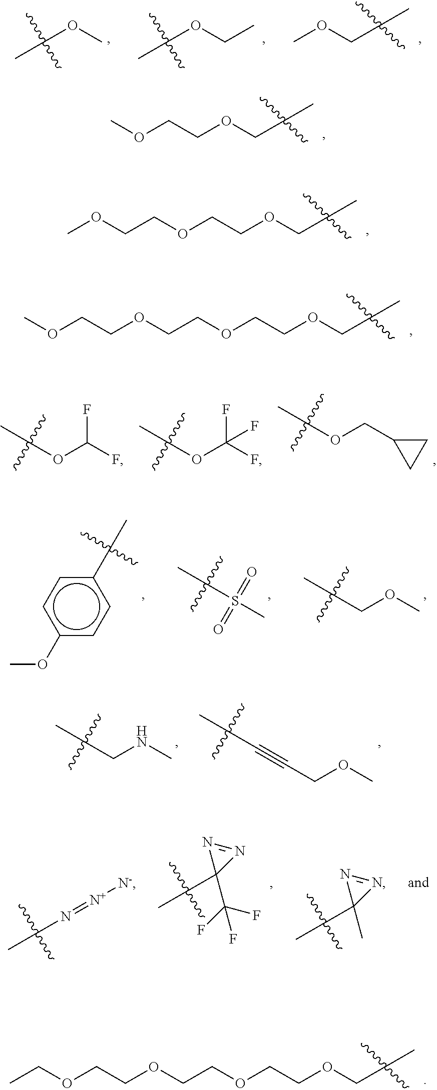

In some embodiments, R² is selected from H, F, Cl, OH, OMe, Me, Et, i-Pr,

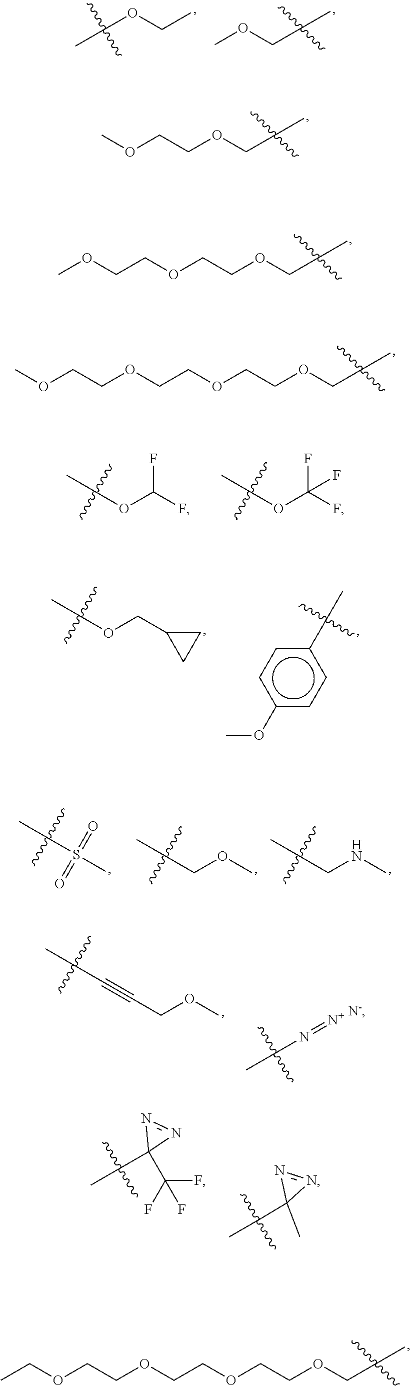

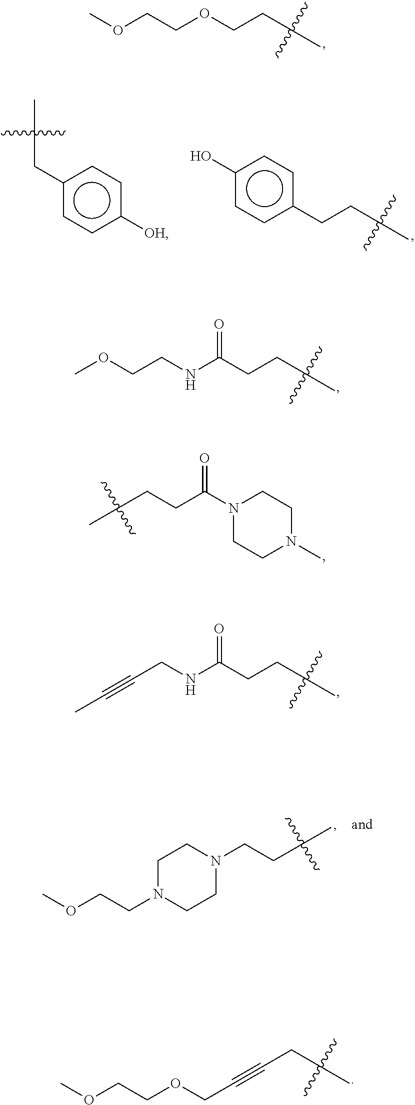

In some embodiments, R² is -L¹- and is selected from

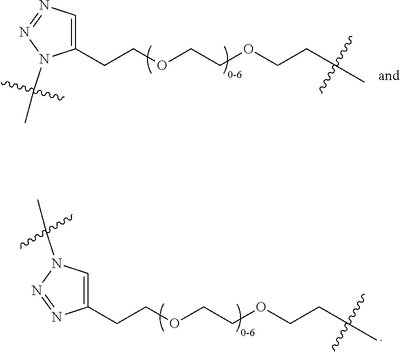

In some embodiments, R² is -L¹- and is selected from

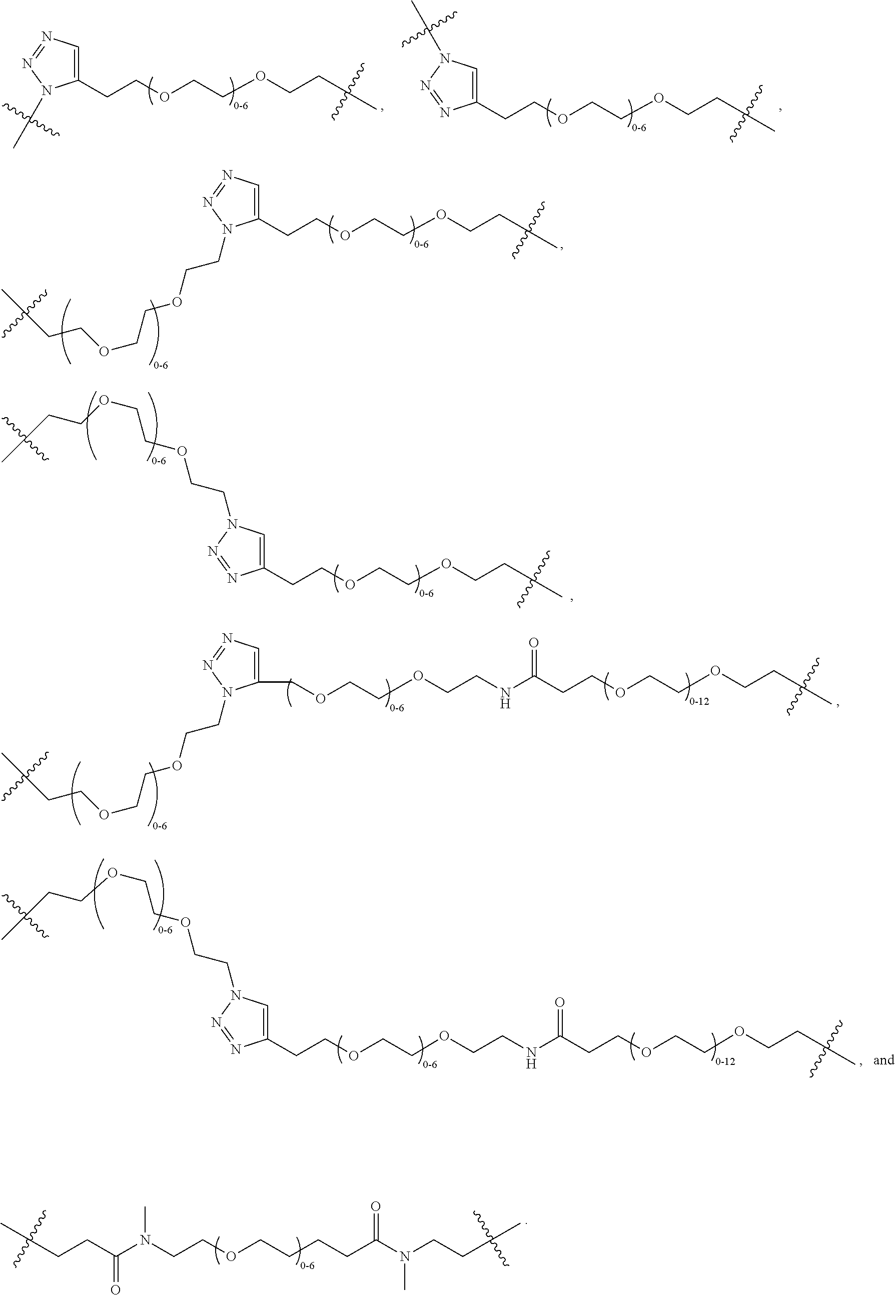

In some embodiments, R² is -L¹- and is selected from

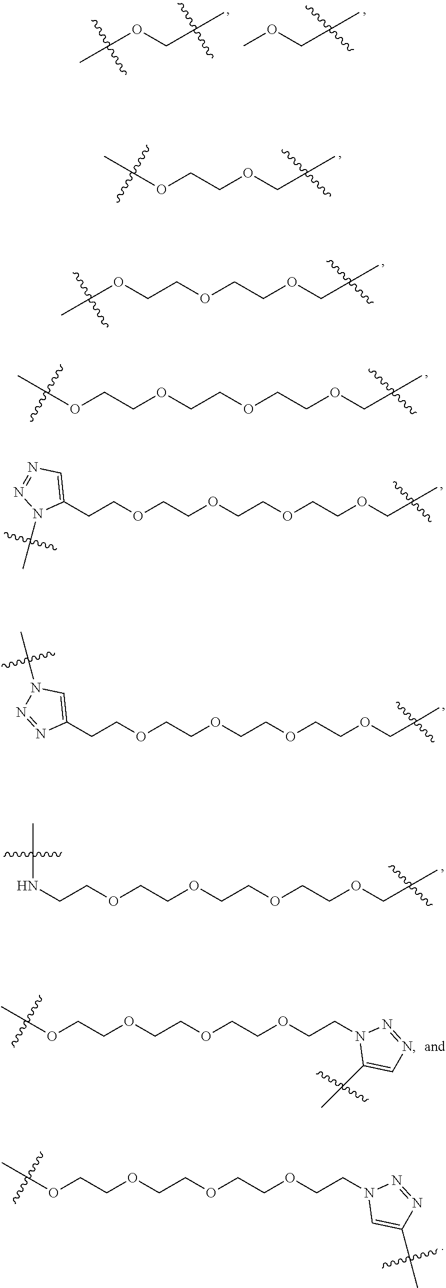

In some embodiments, R² is -L¹- and is selected from

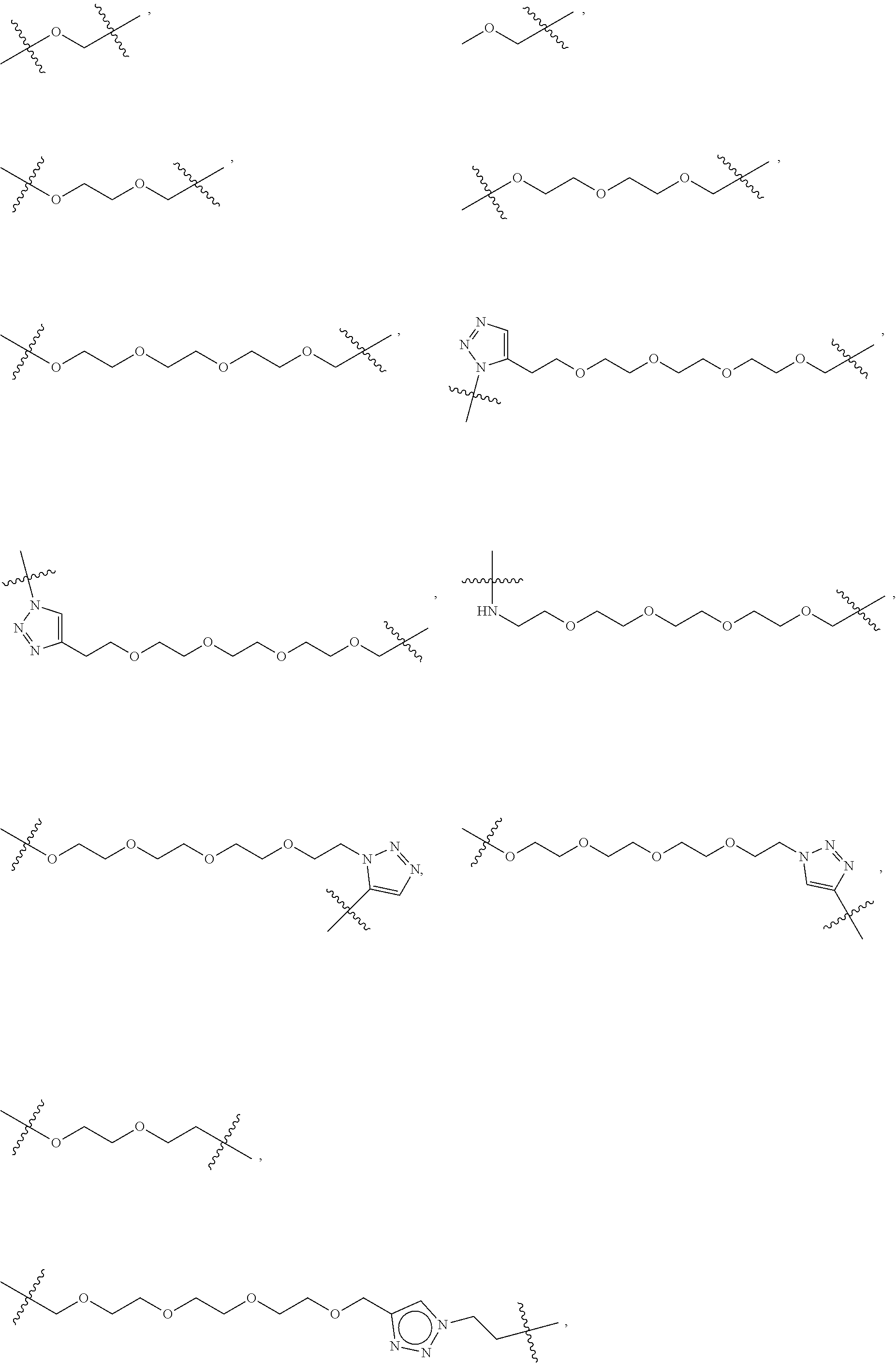

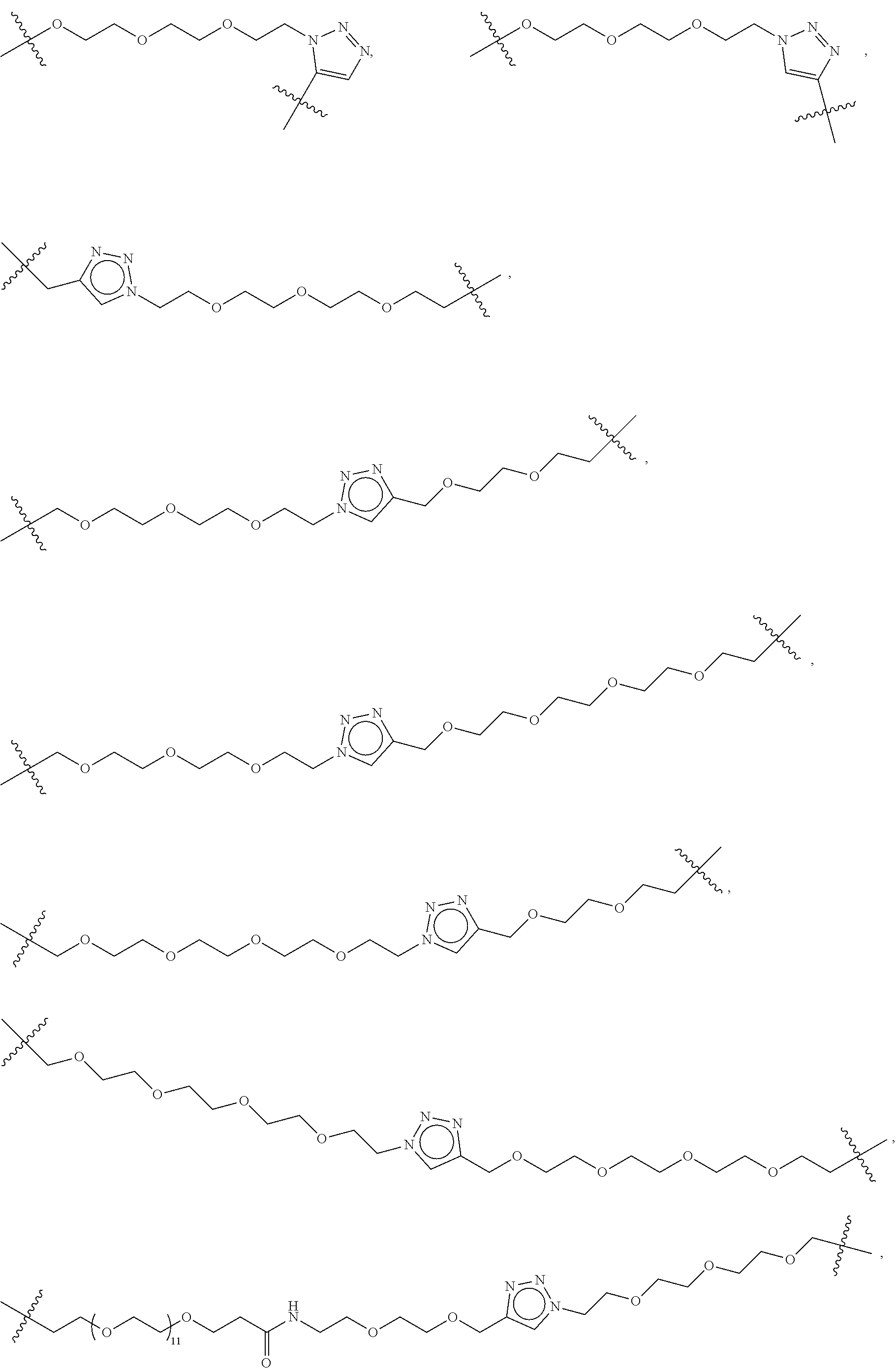

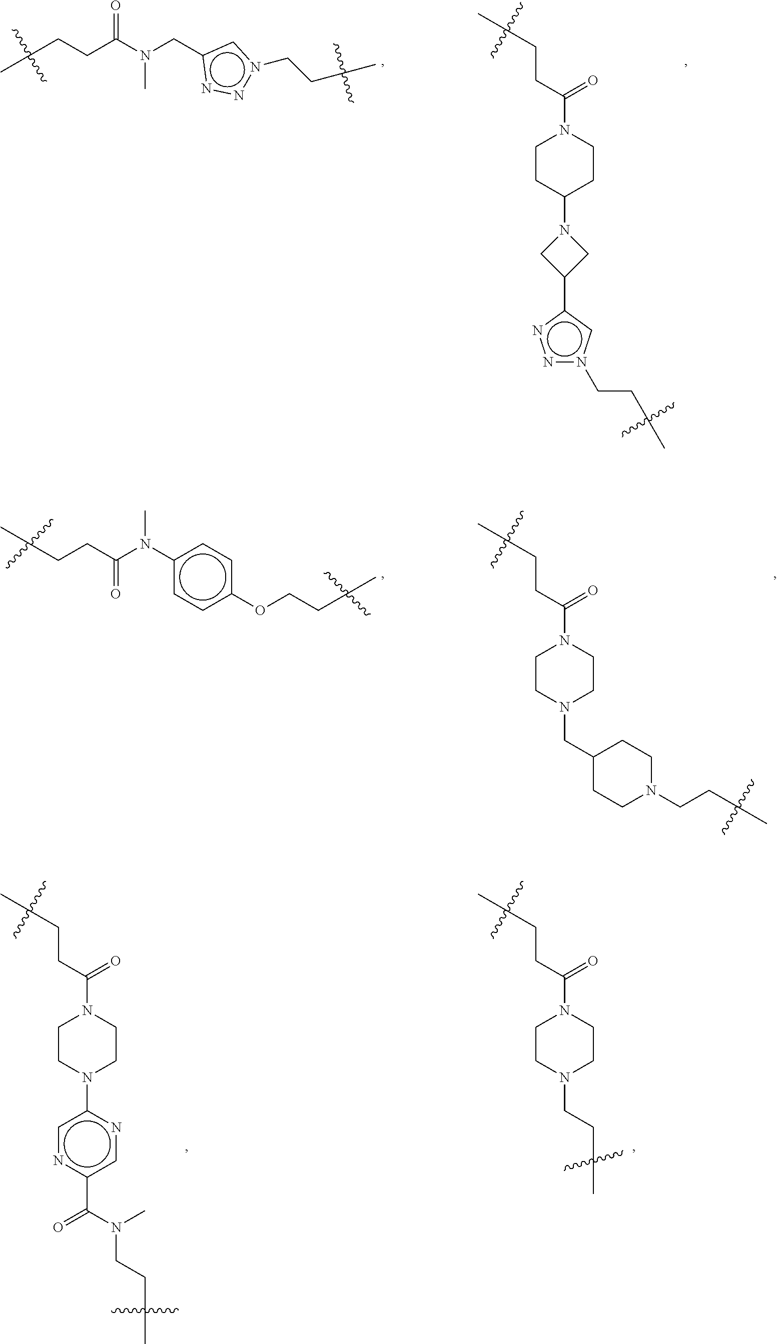

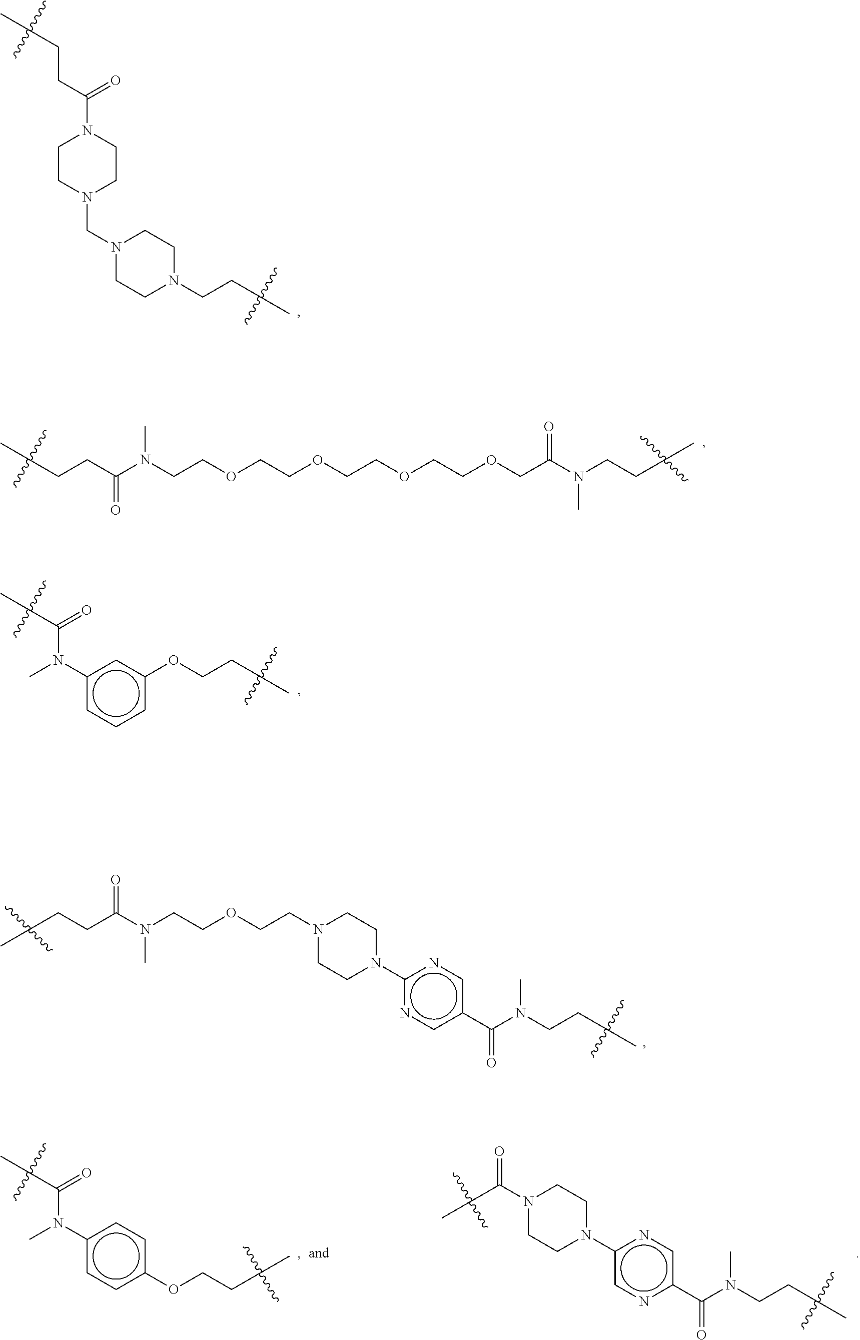

In some embodiments, R² is -L¹- and is selected from one of those in Table 3, below.

In some embodiments, R³ is R. In some embodiments, R³ is halogen. In some embodiments, R³ is —CN. In some embodiments, R³ is —NC. In some embodiments, R³ is —C(O)OR. In some embodiments, R³ is —OC(O)R. In some embodiments, R³ is —C(O)N(R)₂. In some embodiments, R³ is —N(R)C(O)R. In some embodiments, R³ is —N(R)C(O)N(R)₂. In some embodiments, R³ is —OC(O)N(R)₂. In some embodiments, R³ is —N(R)C(O)OR. In some embodiments, R³ is —OR. In some embodiments, R³ is —N(R)₂. In some embodiments, R³ is —NO₂. In some embodiments, R³ is —N₃. In some embodiments, R³ is —SR. In some embodiments, R³ is —S(O)R. In some embodiments, R³ is —S(O)₂R. In some embodiments, R³ is —S(O)₂N(R)₂. In some embodiments, R³ is —NRS(O)₂R. In some embodiments, R³ is -L¹-.

In some embodiments, R³ is hydrogen. In some embodiments, R³ is an optionally substituted C₁₋₆ aliphatic group. In some embodiments, R³ is an optionally substituted 4-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, and sulfur. In some embodiments, R³ is an optionally substituted 5-6 membered monocyclic heteroaromatic ring having 1-4 heteroatoms independently selected from nitrogen, oxygen, and sulfur. In some embodiments, R³ is an optionally substituted 8-10 membered bicyclic heteroaromatic ring having 1-5 heteroatoms independently selected from nitrogen, oxygen, and sulfur.

In some embodiments, R³ is a C₁₋₈ bivalent straight or branched hydrocarbon chain. In some embodiments, R³ is a C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R³ is a C₁₋₁₆ bivalent straight or branched hydrocarbon chain. In some embodiments, R³ is a C₁₋₁₆ bivalent straight or branched hydrocarbon chain wherein 1, 2, 3, 4, 5 or 6 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R³ is selected from H, F, Cl, OH, —OMe, Me, Et, CF₃, CN, —C(O)NH₂, i-Pr,

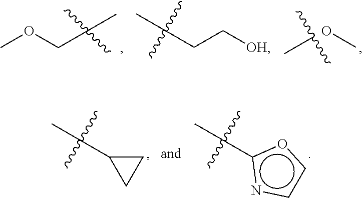

In some embodiments, R³ is selected from

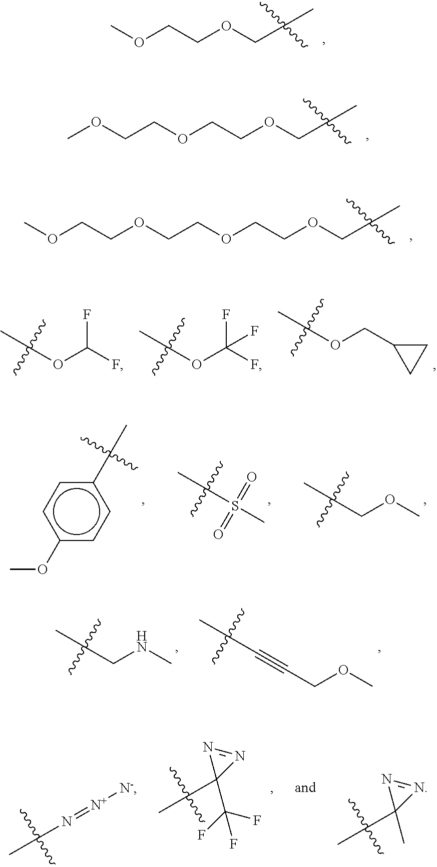

In some embodiments, R³ is selected from

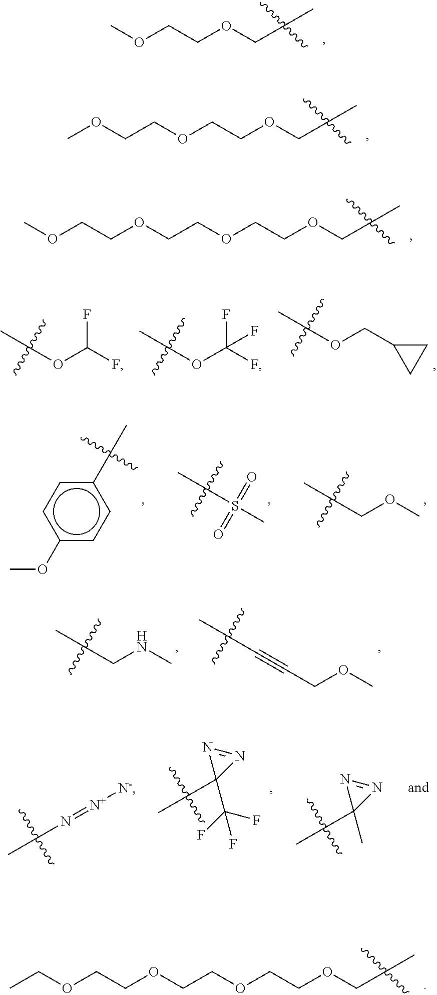

In some embodiments, R³ is selected from

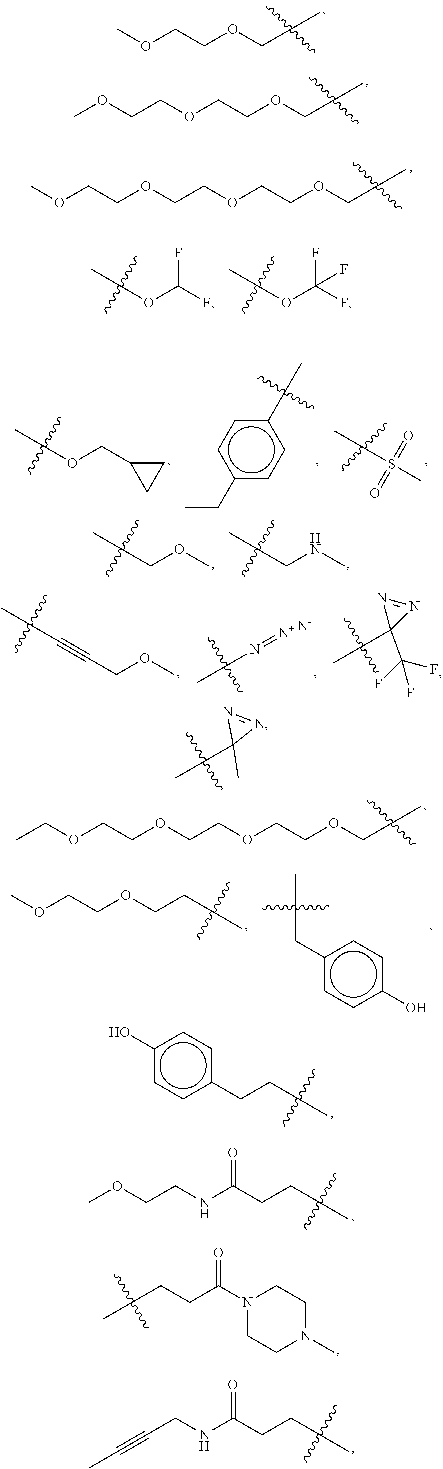

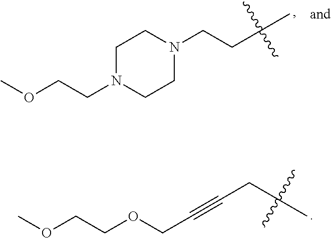

In some embodiments, R³ is -L¹- and is selected from

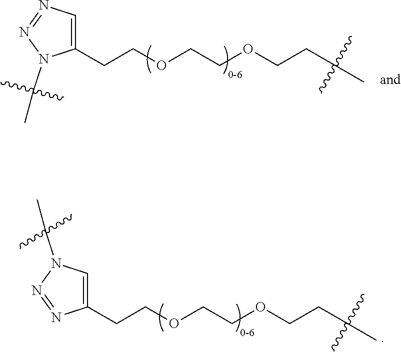

In some embodiments, R³ is -L¹- and is selected from

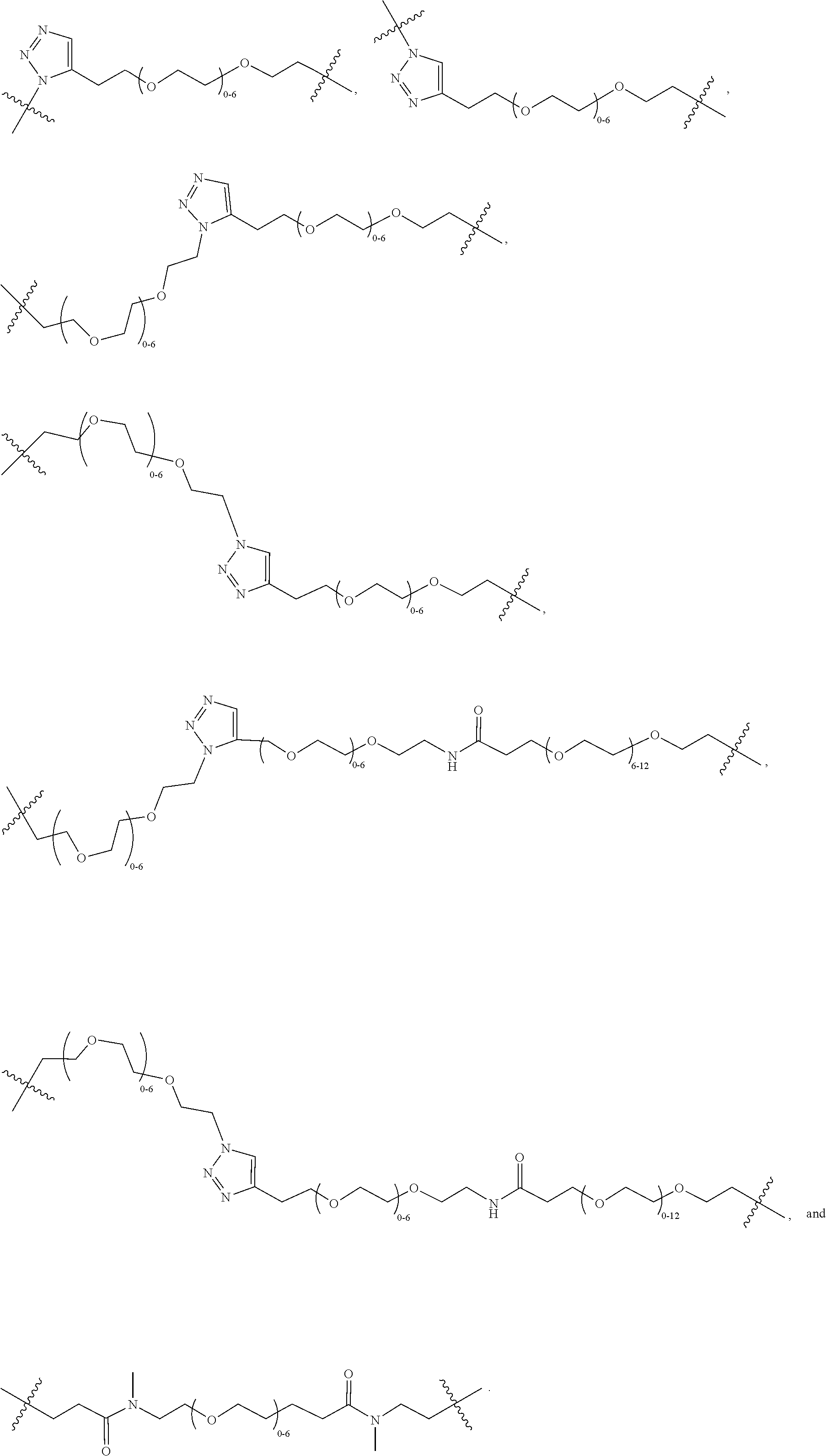

In some embodiments R³ is -L- and is selected from

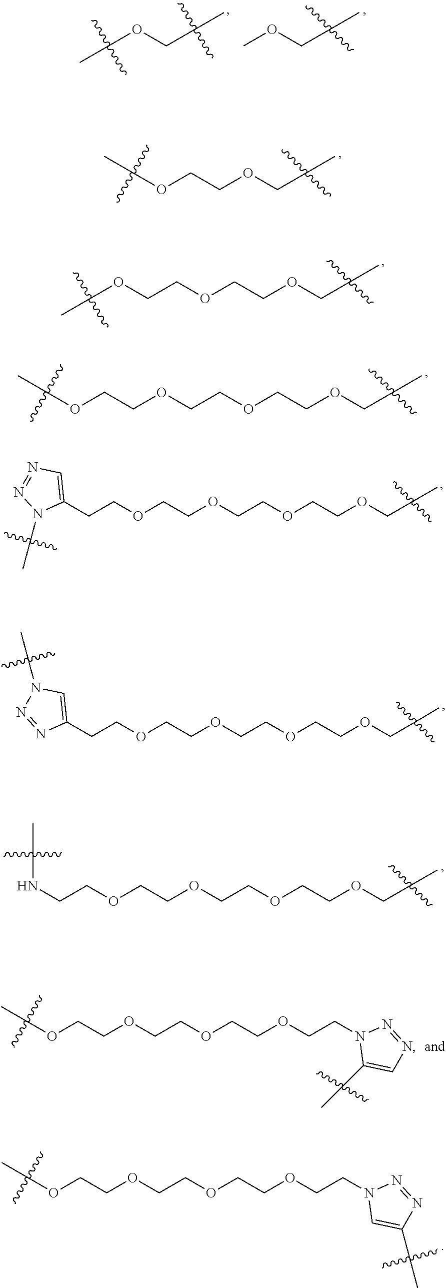

In some embodiments, R³ is -L¹- and is selected from

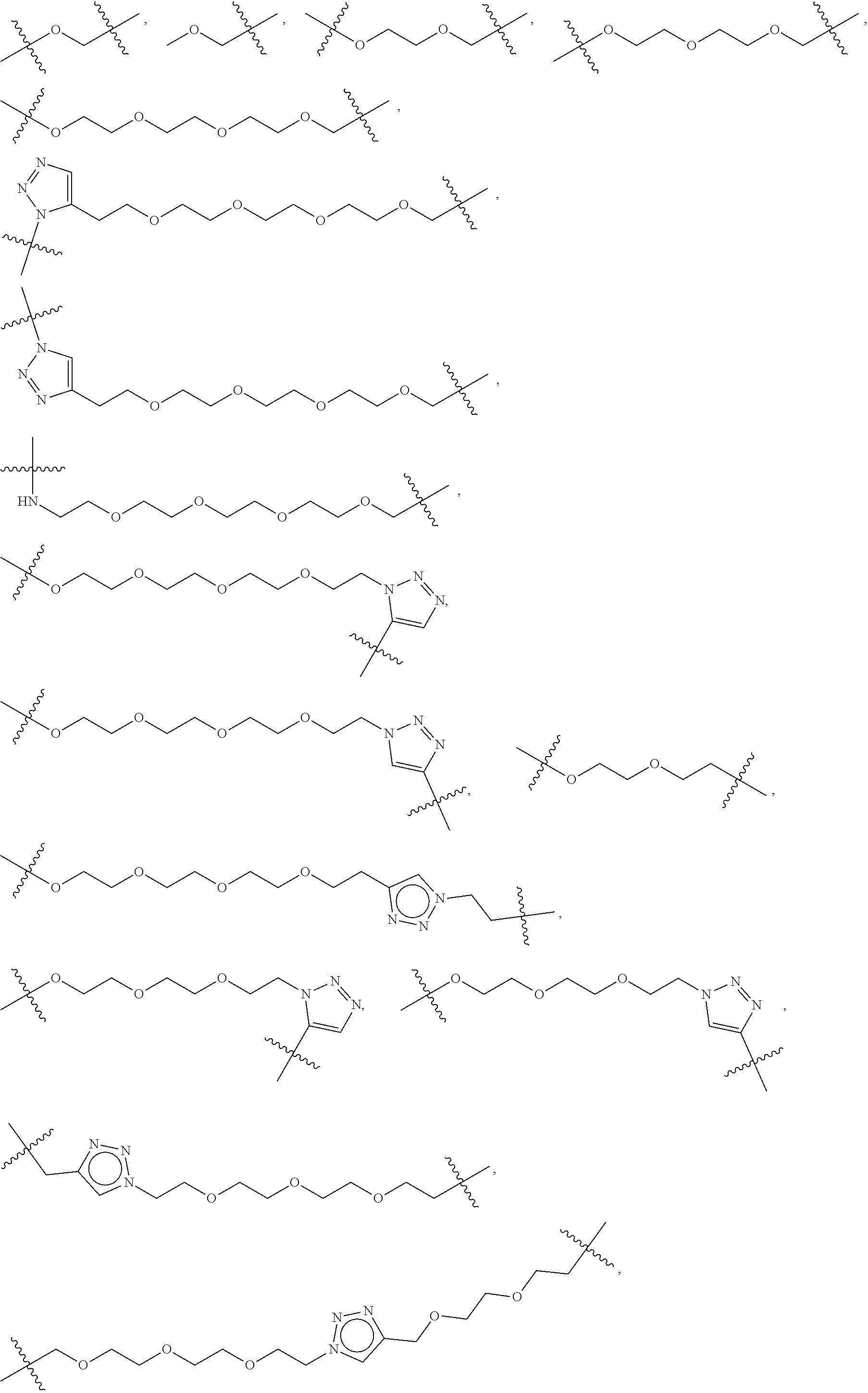

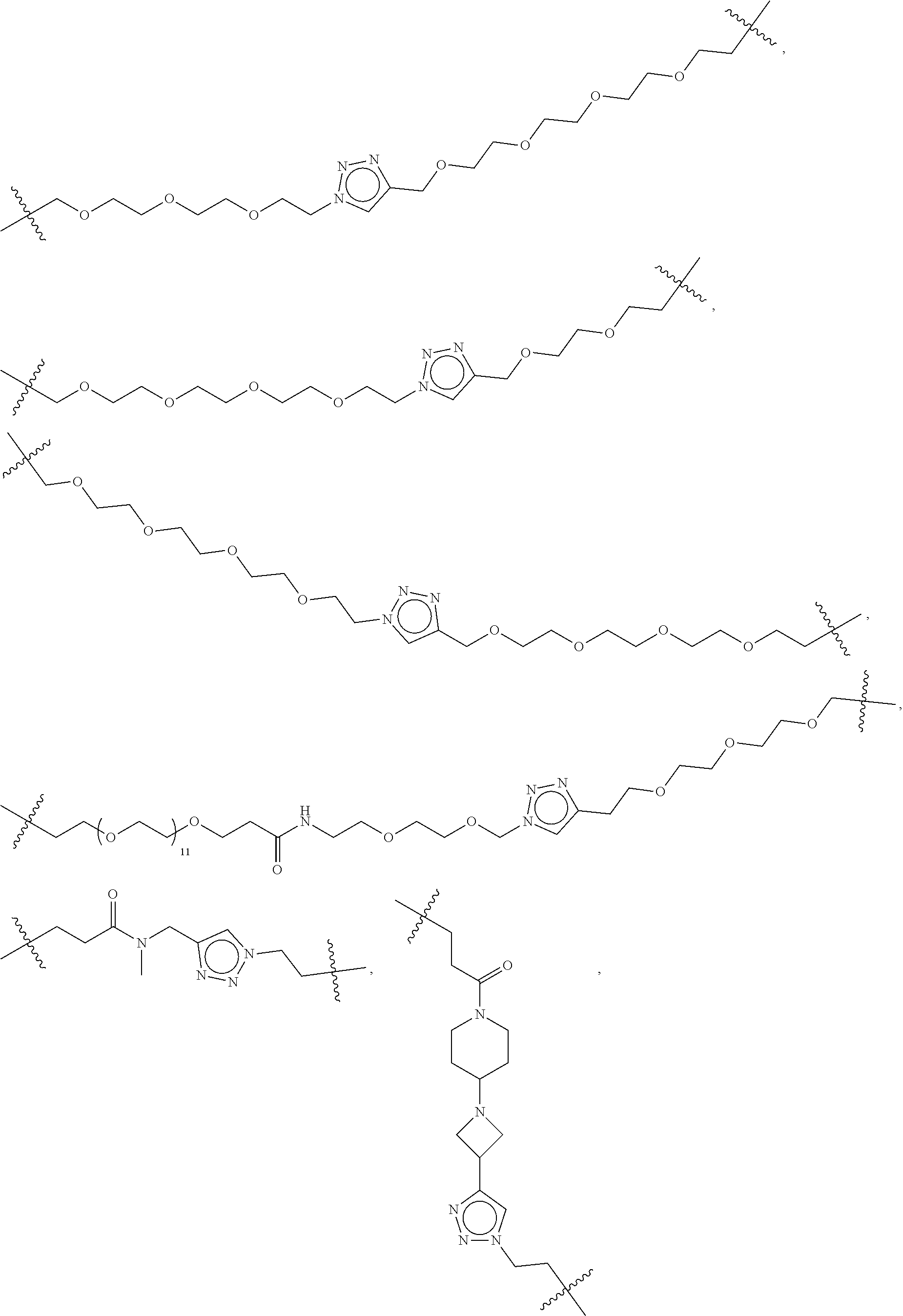

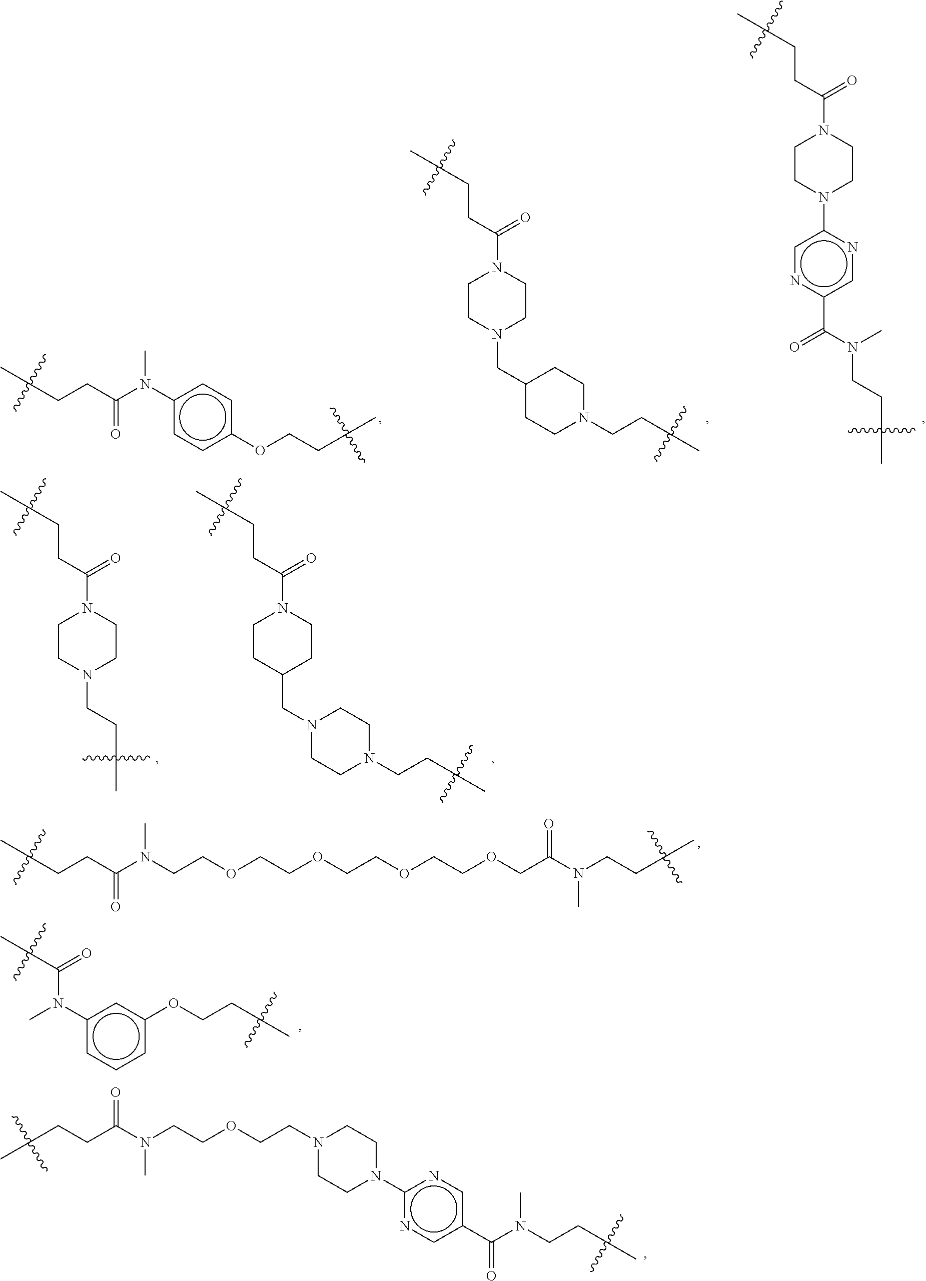

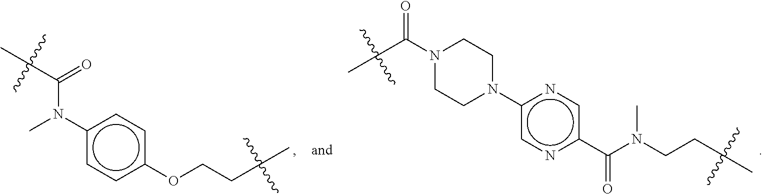

In some embodiments, R³ is -L¹- and is selected from one of those in Table 3, below.

In some embodiments, R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² and R³ taken together with the carbons to which they are attached form a 4-8 membered saturated or partially unsaturated monocyclic carbocyclic ring. In some embodiments, R² and R³ taken together with the carbons to which they are attached form phenyl. In some embodiments, R² and R³ taken together with the carbons to which they are attached form an 8-10 membered bicyclic aromatic carbocyclic ring. In some embodiments, R² and R³ taken together with the carbons to which they are attached form a 4-8 membered saturated or partially unsaturated monocyclic heterocyclic ring having 1-2 heteroatoms independently selected from nitrogen, oxygen, and sulfur. In some embodiments, R² and R³ taken together with the carbons to which they are attached form a 5-6 membered monocyclic heteroaromatic ring having 1-4 heteroatoms independently selected from nitrogen, oxygen, and sulfur. In some embodiments, R² and R³ taken together with the carbons to which they are attached form an 8-10 membered bicyclic heteroaromatic ring having 1-5 heteroatoms independently selected from nitrogen, oxygen, and sulfur.

In some embodiments, R² and R³ taken together with the carbons to which they are attached form a cyclohexanylene ring. In some embodiments, R² and R³ taken together with the carbons to which they are attached form a cyclopentanylene ring.

In some embodiments, R² and R³ are selected from those depicted in Table 1, below.

In some embodiments, R² and R³ are selected from those depicted in Table 1A, below.

In some embodiments, R² and R³ are selected from those depicted in Table 1B, below.

In some embodiments, R² and R³ are selected from those depicted in Table 1C, below.

As defined generally above, each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R⁴ is R. In some embodiments, R⁴ is halogen. In some embodiments, R⁴ is ═O. In some embodiments, R⁴ is —CN. In some embodiments, R⁴ is —NC. In some embodiments, R⁴ is —C(O)OR. In some embodiments, R⁴ is —OC(O)R. In some embodiments, R⁴ is —C(O)N(R)₂. In some embodiments, R⁴ is —N(R)C(O)R. In some embodiments, R⁴ is —N(R)C(O)N(R)₂. In some embodiments, R⁴ is —OC(O)N(R)₂. In some embodiments, R⁴ is —N(R)C(O)OR. In some embodiments, R⁴ is —OR. In some embodiments, R⁴ is —N(R)₂. In some embodiments, R⁴ is —NO₂. In some embodiments, R⁴ is —N₃. In some embodiments, R⁴ is —SR. In some embodiments, R⁴ is —S(O)R. In some embodiments, R⁴ is —S(O)₂R. In some embodiments, R⁴ is —S(O)₂N(R)₂. In some embodiments, R⁴ is —NRS(O)₂R. In some embodiments, R⁴ is C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R⁴ is hydrogen. In some embodiments, R⁴ is an optionally substituted C₁₋₆ aliphatic group. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, and sulfur. In some embodiments, R⁴ is an optionally substituted 5-6 membered monocyclic heteroaromatic ring having 1-4 heteroatoms independently selected from nitrogen, oxygen, and sulfur. In some embodiments, R⁴ is an optionally substituted 8-10 membered bicyclic heteroaromatic ring having 1-5 heteroatoms independently selected from nitrogen, oxygen, and sulfur.

In some embodiments, R⁴ is selected from H, ═O, Me, Et, iPr,

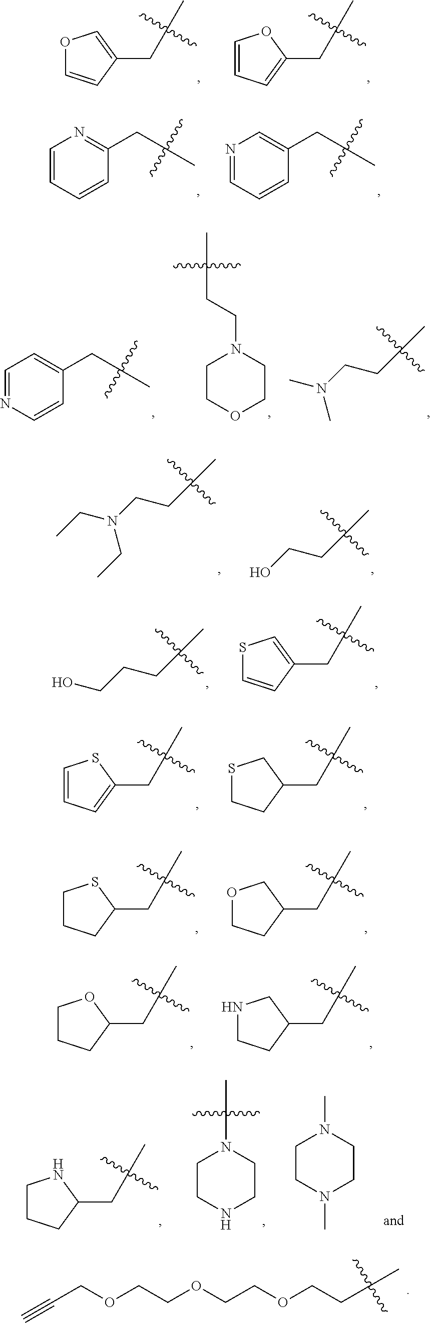

In some embodiments, R⁴ is selected from those depicted in Table 1, below.

In some embodiments, R⁴ is selected from those depicted in Table 1A, below.

In some embodiments, R⁴ is selected from those depicted in Table 1B, below.

In some embodiments, R⁴ is selected from those depicted in Table 1C, below.

As defined generally above, each R⁵ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁵ is R. In some embodiments, R⁵ is halogen. In some embodiments, R⁵ is —CN. In some embodiments, R⁵ is —NC. In some embodiments, R⁵ is —C(O)OR. In some embodiments, R⁵ is —OC(O)R. In some embodiments, R⁵ is —C(O)N(R)₂. In some embodiments, R⁵ is —N(R)C(O)R. In some embodiments, R⁵ is —N(R)C(O)N(R)₂. In some embodiments, R⁵ is —OC(O)N(R)₂. In some embodiments, R⁵ is —N(R)C(O)OR. In some embodiments, R⁵ is —OR. In some embodiments, R⁵ is —N(R)₂. In some embodiments, R⁵ is —NO₂. In some embodiments, R⁵ is —N₃. In some embodiments, R⁵ is —SR. In some embodiments, R⁵ is —S(O)R. In some embodiments, R⁵ is —S(O)₂R. In some embodiments, R⁵ is —S(O)₂N(R)₂. In some embodiments, R⁵ is —NRS(O)₂R.

In some embodiments, R⁵ is selected from those depicted in Table 1, below.

In some embodiments, R⁵ is selected from those depicted in Table 1A, below.

In some embodiments, R⁵ is selected from those depicted in Table 1B, below.

In some embodiments, R⁵ is selected from those depicted in Table 1C, below.

As defined generally above, -L²- is

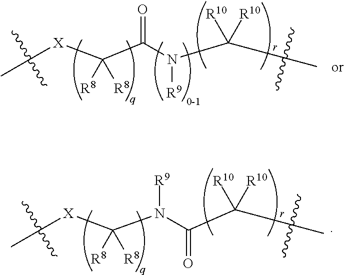

In some embodiments, -L²- is

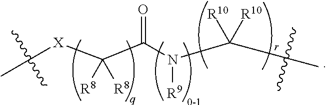

In some embodiments, -L²- is

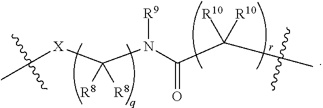

In some embodiments, -L²- is

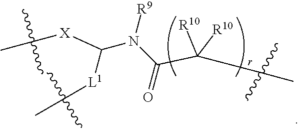

In some embodiments, -L²- is

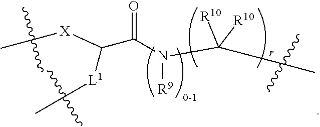

In some embodiments, -L²- is

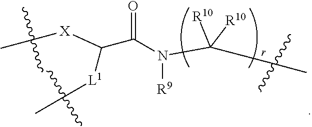

As defined generally above, each R⁶ and R⁷ is independently —R, halogen, —CN, —NC, —C(O)OR, —OC(O)R, —OR, —N(R)₂, or —SR.

In some embodiments, R⁶ is R. In some embodiments, R⁶ is halogen. In some embodiments, R⁶ is —CN. In some embodiments, R⁶ is —NC. In some embodiments, R⁶ is —C(O)OR. In some embodiments, R⁶ is —OC(O)R. In some embodiments, R⁶ is —OR. In some embodiments, R⁶ is —N(R)₂. In some embodiments, R⁶ is —SR.

In some embodiments, R⁶ is hydrogen. In some embodiments, R⁶ is an optionally substituted C₁₋₆ aliphatic group. In some embodiments, R⁶ is an optionally substituted 3-8 membered saturated or partially unsaturated monocyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 4-8 membered saturated or partially unsaturated monocyclic heterocyclic ring having 1-2 heteroatoms independently selected from nitrogen, oxygen, and sulfur. In some embodiments, R⁶ is an optionally substituted 5-6 membered monocyclic heteroaromatic ring having 1-4 heteroatoms independently selected from nitrogen, oxygen, and sulfur. In some embodiments, R⁶ is an optionally substituted 8-10 membered bicyclic heteroaromatic ring having 1-5 heteroatoms independently selected from nitrogen, oxygen, and sulfur.

In some embodiments, R⁶ is selected from H, Me, Et, iPr, F, and OH.

In some embodiments, R⁶ is selected from those depicted in Table 1, below.

In some embodiments, R⁶ is selected from those depicted in Table 1A, below.

In some embodiments, R⁶ is selected from those depicted in Table 1B, below.

In some embodiments, R⁶ is selected from those depicted in Table 1C, below.

In some embodiments, R⁷ is R. In some embodiments, R⁷ is halogen. In some embodiments, R⁷ is —CN. In some embodiments, R⁷ is —NC. In some embodiments, R⁷ is —C(O)OR. In some embodiments, R⁷ is —OC(O)R. In some embodiments, R⁷ is —OR. In some embodiments, R⁷ is —N(R)₂. In some embodiments, R⁷ is —SR.

In some embodiments, R⁷ is hydrogen. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, and sulfur. In some embodiments, R⁷ is an optionally substituted 5-6 membered monocyclic heteroaromatic ring having 1-4 heteroatoms independently selected from nitrogen, oxygen, and sulfur. In some embodiments, R⁷ is an optionally substituted 8-10 membered bicyclic heteroaromatic ring having 1-5 heteroatoms independently selected from nitrogen, oxygen, and sulfur.

In some embodiments, R⁷ is selected from H, Me, Et, iPr, F and OH.

In some embodiments, R⁷ is selected from those depicted in Table 1, below.

In some embodiments, R⁷ is selected from those depicted in Table 1A, below.

In some embodiments, R⁷ is selected from those depicted in Table 1B, below.

In some embodiments, R⁷ is selected from those depicted in Table 1C, below.

As defined generally above, X is a bond, NR, O, CR⁶R⁷—C(O)—, —S—, or —S(O)₂—.

In some embodiments, X is selected from a bond, NH, O, S, SO₂, C(O), NMe, CF₂, CHMe, CHOH, C(Me)₂ and C(Me)OH.

In some embodiments, X is selected from those depicted in Table 1, below.

In some embodiments, X is selected from those depicted in Table 1A, below.

In some embodiments, X is selected from those depicted in Table 1B, below.

In some embodiments, X is selected from those depicted in Table 1C, below.

As defined generally above, each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, -L¹- or two R¹ taken together with the carbon atom to which they are attached, form a 3-6 membered carbocyclic ring.

In some embodiments, R⁸ is R. In some embodiments, R⁸ is halogen. In some embodiments, R⁸ is —CN. In some embodiments, R⁸ is —NC. In some embodiments, R⁸ is —C(O)OR. In some embodiments, R⁸ is —OC(O)R. In some embodiments, R⁸ is —C(O)N(R)₂. In some embodiments, R⁸ is —N(R)C(O)R. In some embodiments, R⁸ is —N(R)C(O)N(R)₂. In some embodiments, R⁸ is —OC(O)N(R)₂. In some embodiments, R⁸ is —N(R)C(O)OR. In some embodiments, R⁸ is —OR. In some embodiments, R⁸ is —N(R)₂. In some embodiments, R⁸ is —NO₂. In some embodiments, R⁸ is —N₃. In some embodiments, R⁸ is —SR. In some embodiments, R⁸ is —S(O)R. In some embodiments, R⁸ is —S(O)₂R. In some embodiments, R⁸ is —S(O)₂N(R)₂. In some embodiments, R⁸ is —NRS(O)₂R. In some embodiments, R⁸ is -L¹-. In some embodiments, two R⁸ taken together with the carbon atom to which they are attached, form a 3-6 membered carbocyclic ring.

In some embodiments, R⁸ is hydrogen. In some embodiments, R⁸ is an optionally substituted C₁₋₆ aliphatic group. In some embodiments, R⁸ is an optionally substituted 3-8 membered saturated or partially unsaturated monocyclic carbocyclic ring. In some embodiments, R⁸ is an optionally substituted phenyl. In some embodiments, R⁸ is an optionally substituted 8-10 membered bicyclic aromatic carbocyclic ring. In some embodiments, R⁸ is an optionally substituted 4-8 membered saturated or partially unsaturated monocyclic heterocyclic ring having 1-2 heteroatoms independently selected from nitrogen, oxygen, and sulfur. In some embodiments, R⁸ is an optionally substituted 5-6 membered monocyclic heteroaromatic ring having 1-4 heteroatoms independently selected from nitrogen, oxygen, and sulfur. In some embodiments, R⁸ is an optionally substituted 8-10 membered bicyclic heteroaromatic ring having 1-5 heteroatoms independently selected from nitrogen, oxygen, and sulfur.

In some embodiments, R⁸ is a C₁₋₈ bivalent straight or branched hydrocarbon chain. In some embodiments, R⁸ is a C₁₋₈ bivalent straight or branched hydrocarbon chain wherein 1, 2, 3 or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, R⁸ is a C₁₋₁₆ bivalent straight or branched hydrocarbon chain. In some embodiments, R₈ is a C₁₋₁₆ bivalent straight or branched hydrocarbon chain wherein 1, 2, 3, 4, 5 or 6 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-.

In some embodiments, one instance of —C(R⁸)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring. In some embodiments, one instance of —C(R⁸)₂— is optionally replaced by phenyl. In some embodiments, one instance of —C(R⁸)₂— is optionally replaced by an 8-10 membered bicyclic aromatic carbocyclic ring. In some embodiments, one instance of —C(R⁸)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R⁸)₂— is optionally replaced by a 5-6 membered monocyclic heteroaromatic ring having 1-4 heteroatoms independently selected from nitrogen, oxygen, and sulfur. In some embodiments, one instance of —C(R⁸)₂— is optionally replaced by an 8-10 membered bicyclic heteroaromatic ring having 1-5 heteroatoms independently selected from nitrogen, oxygen, and sulfur.

In some embodiments, one instance of —C(R⁸)₂— is optionally replaced by

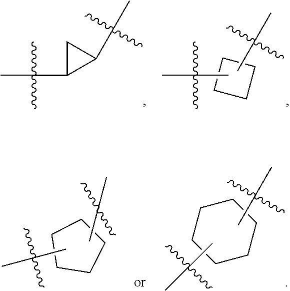

In some embodiments, R⁸ is selected from H, F, Cl, OH, —OMe, Me, Et, i-Pr,

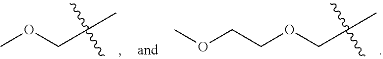

In some embodiments, R⁸ is selected from H, F, Cl, OH, —OMe, Me, Et, i-Pr,

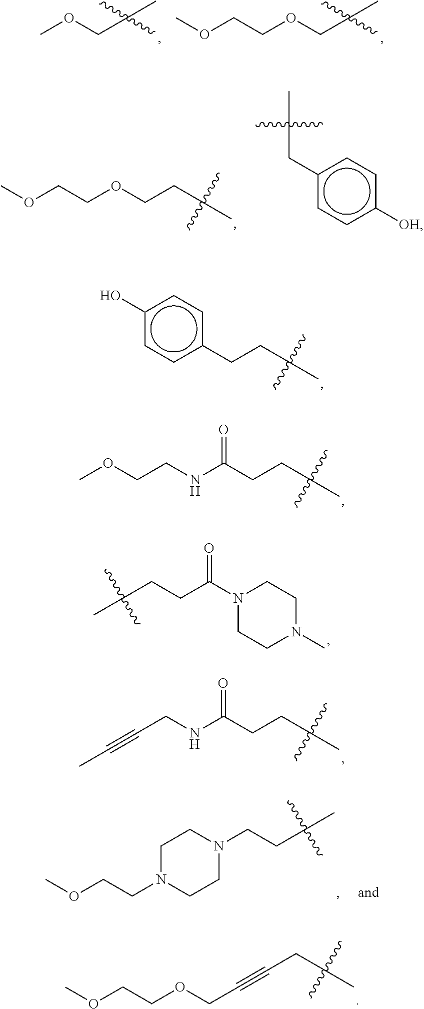

In some embodiments, R⁸ is -L¹- and is selected from

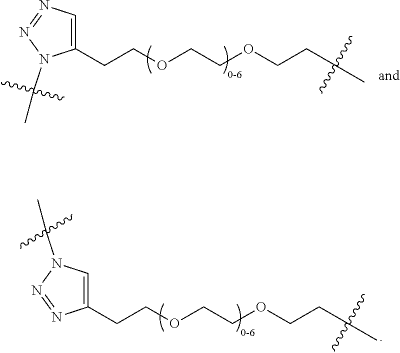

In some embodiments, R⁸ is -L¹- and is selected from

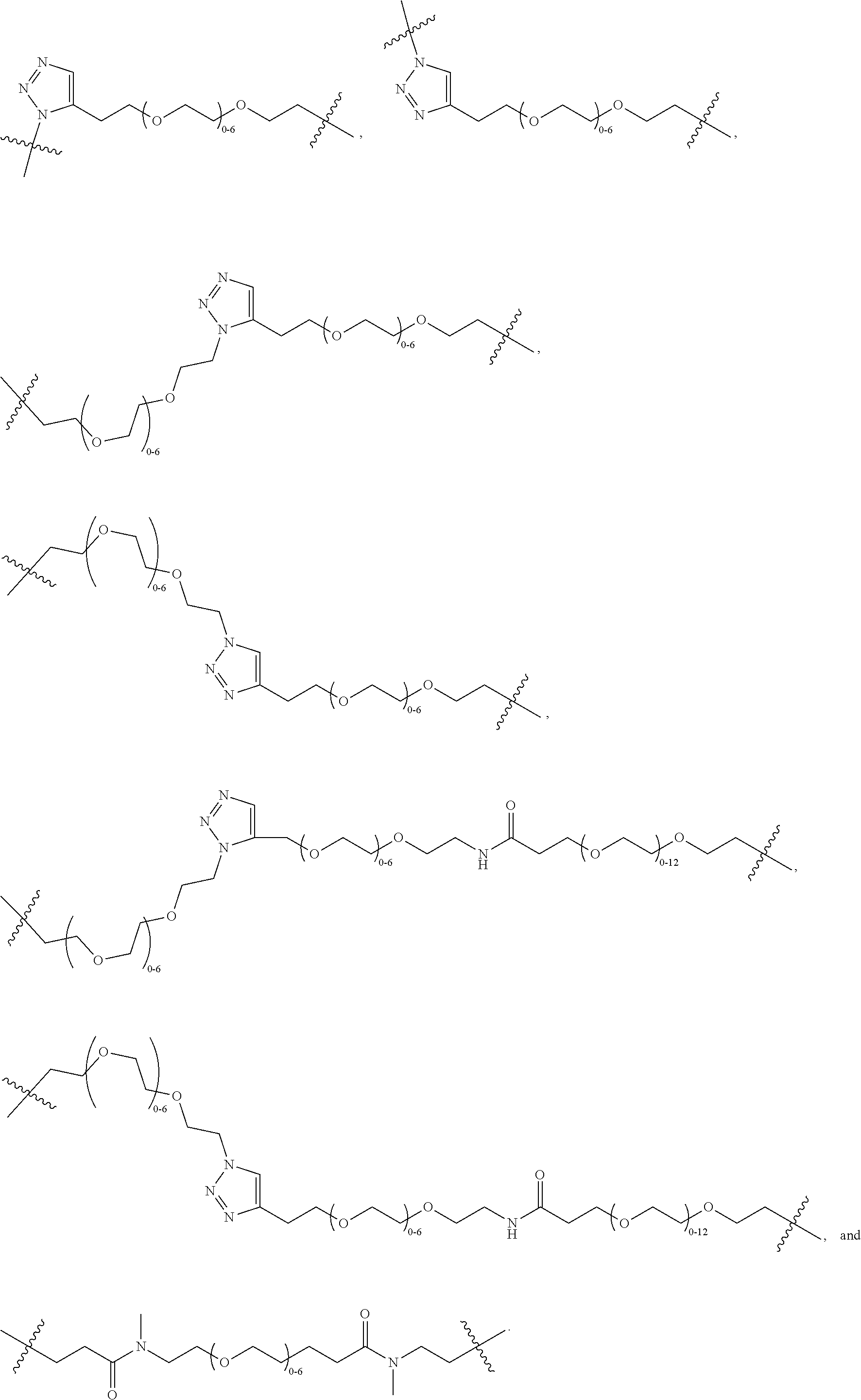

In some embodiments, R⁸ is -L¹- and is selected from

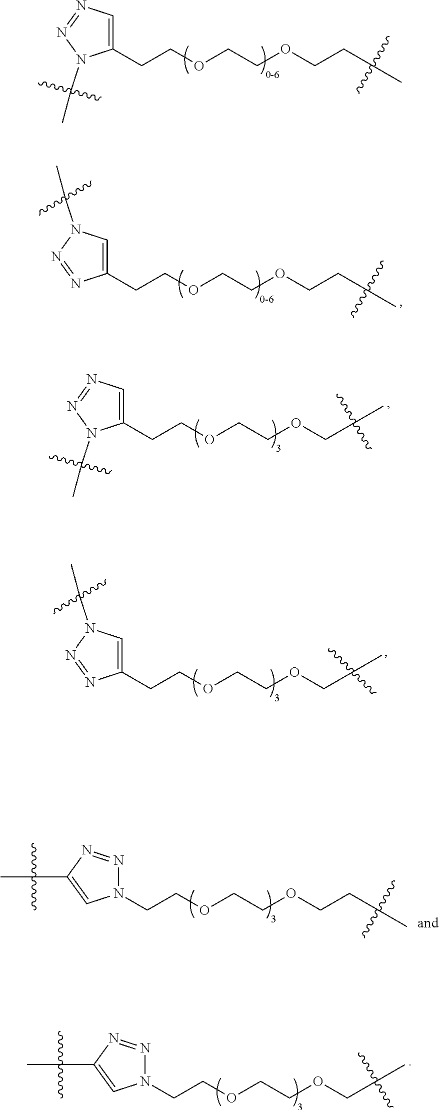

In some embodiments, R⁸ is -L¹- and is selected from

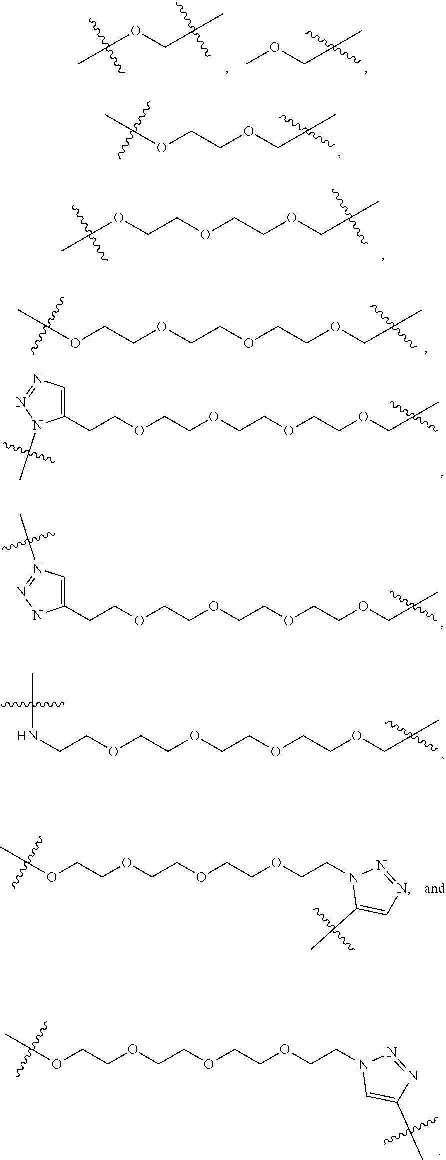

In some embodiments, R⁸ is -L¹- and is selected from

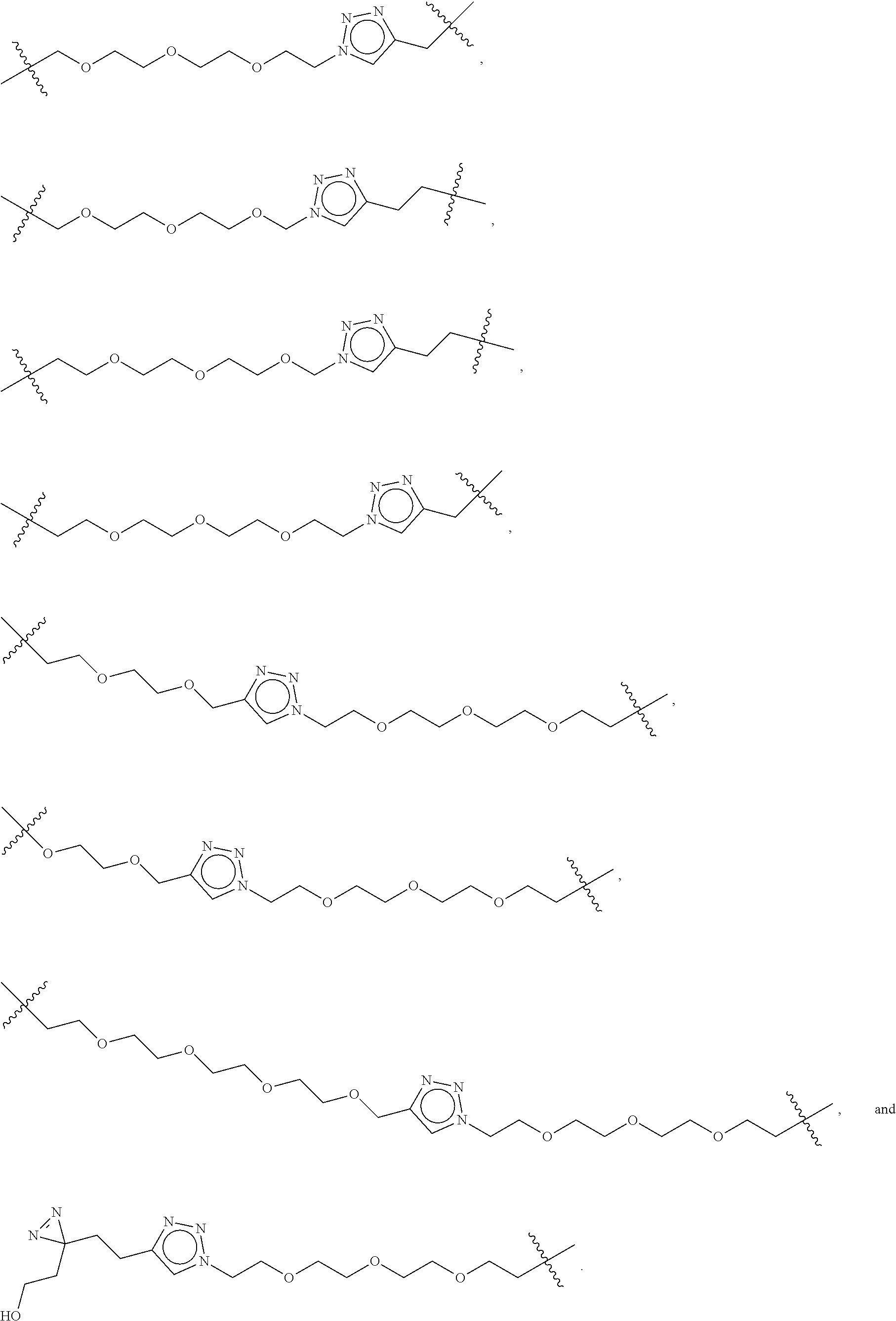

In some embodiments, R⁸ is -L¹- and is selected from

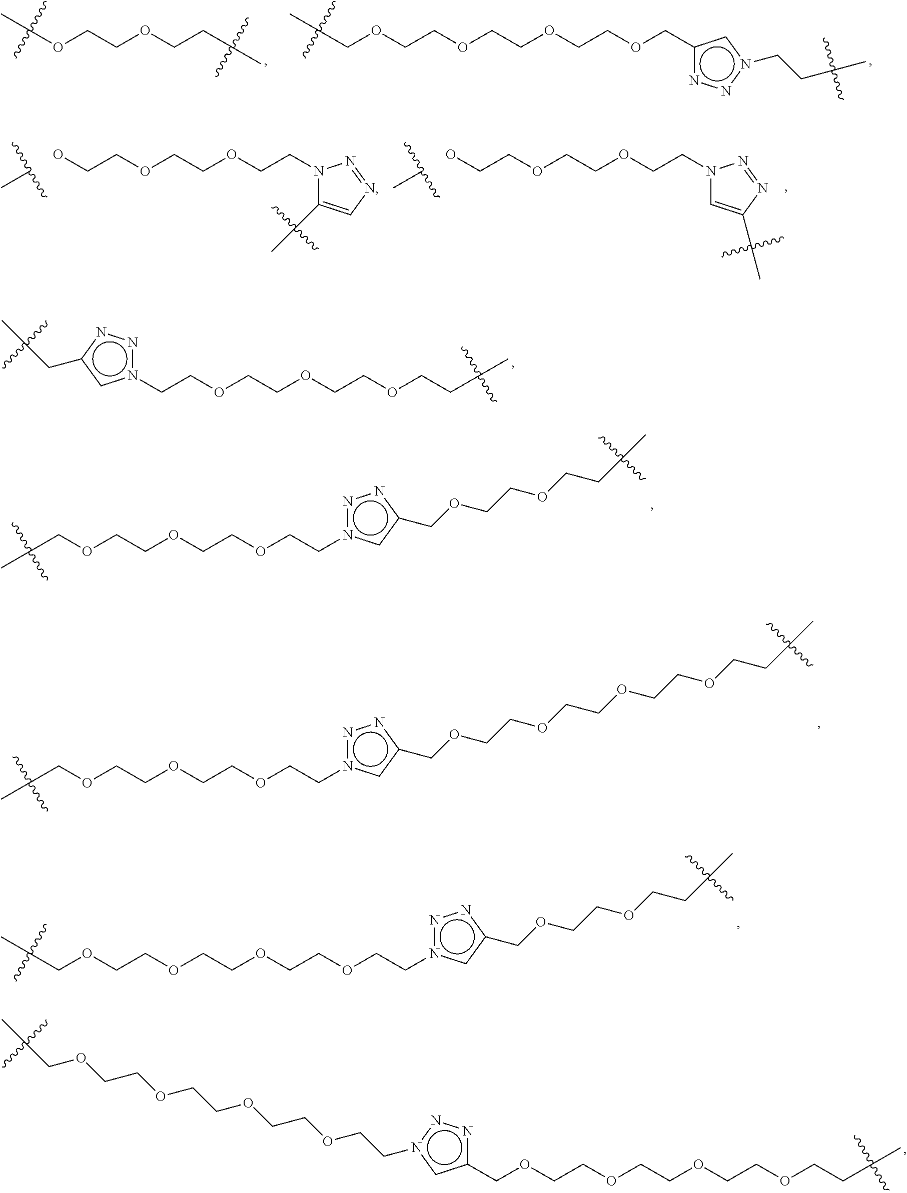

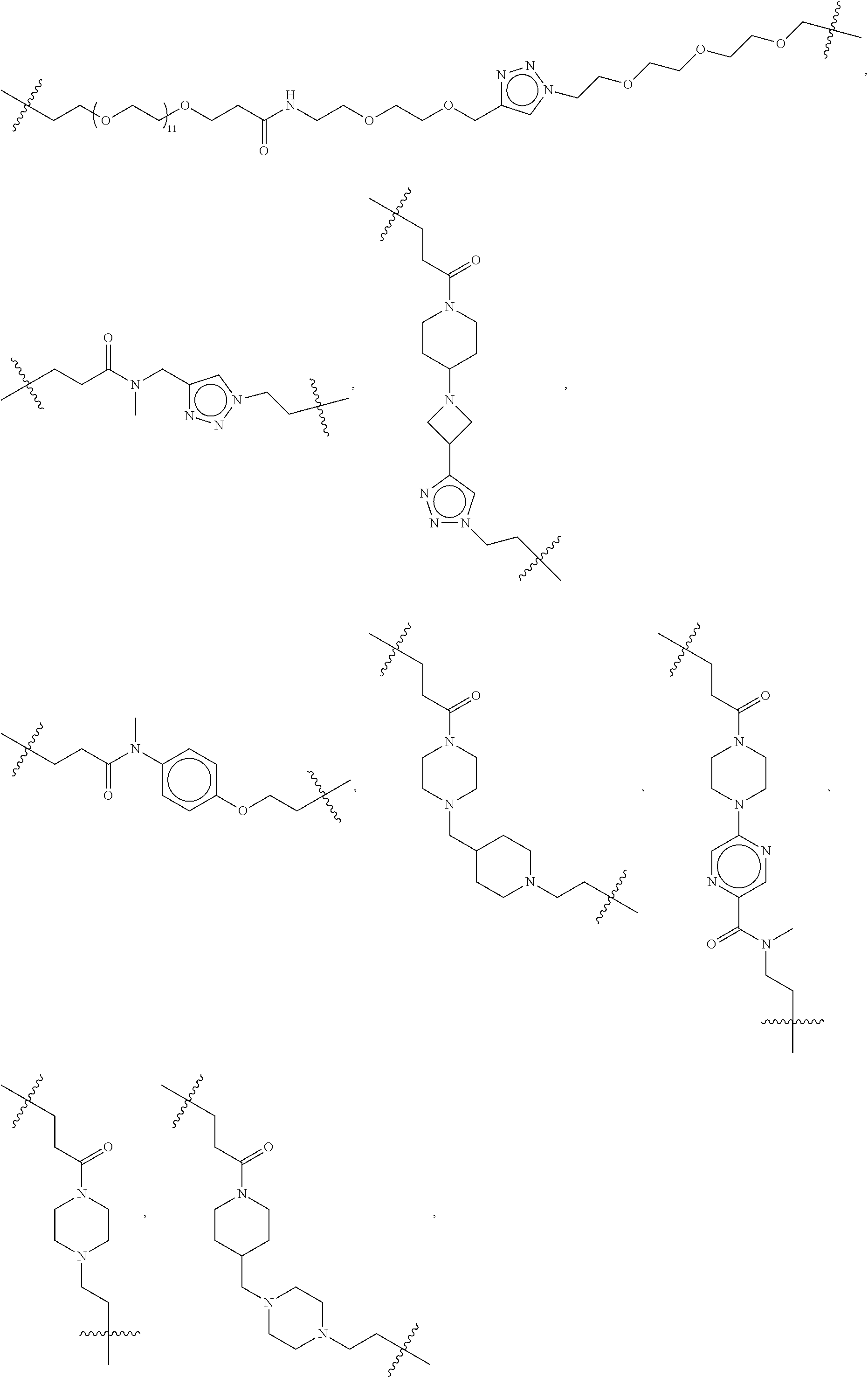

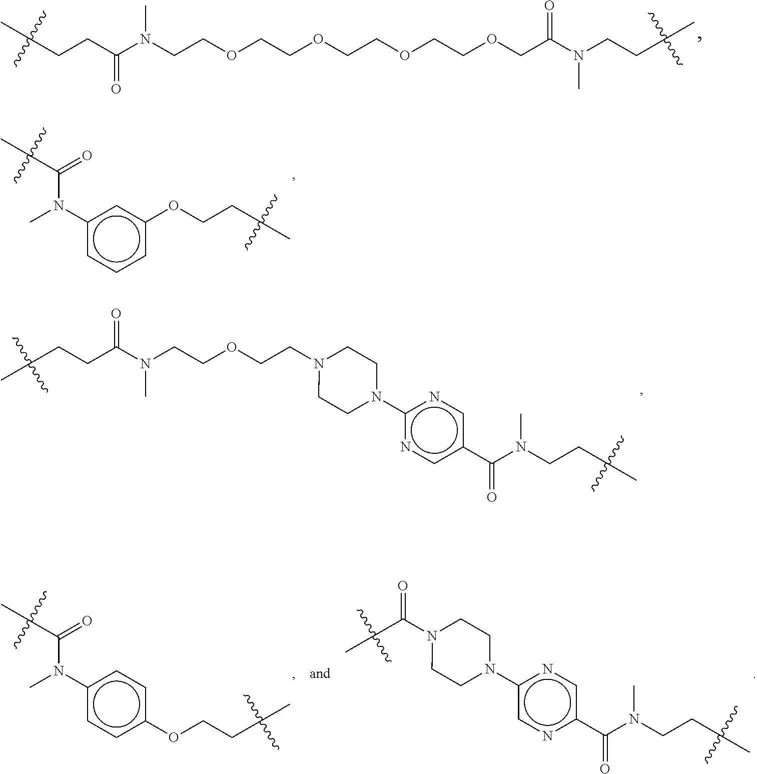

In some embodiments, -L¹- is selected from

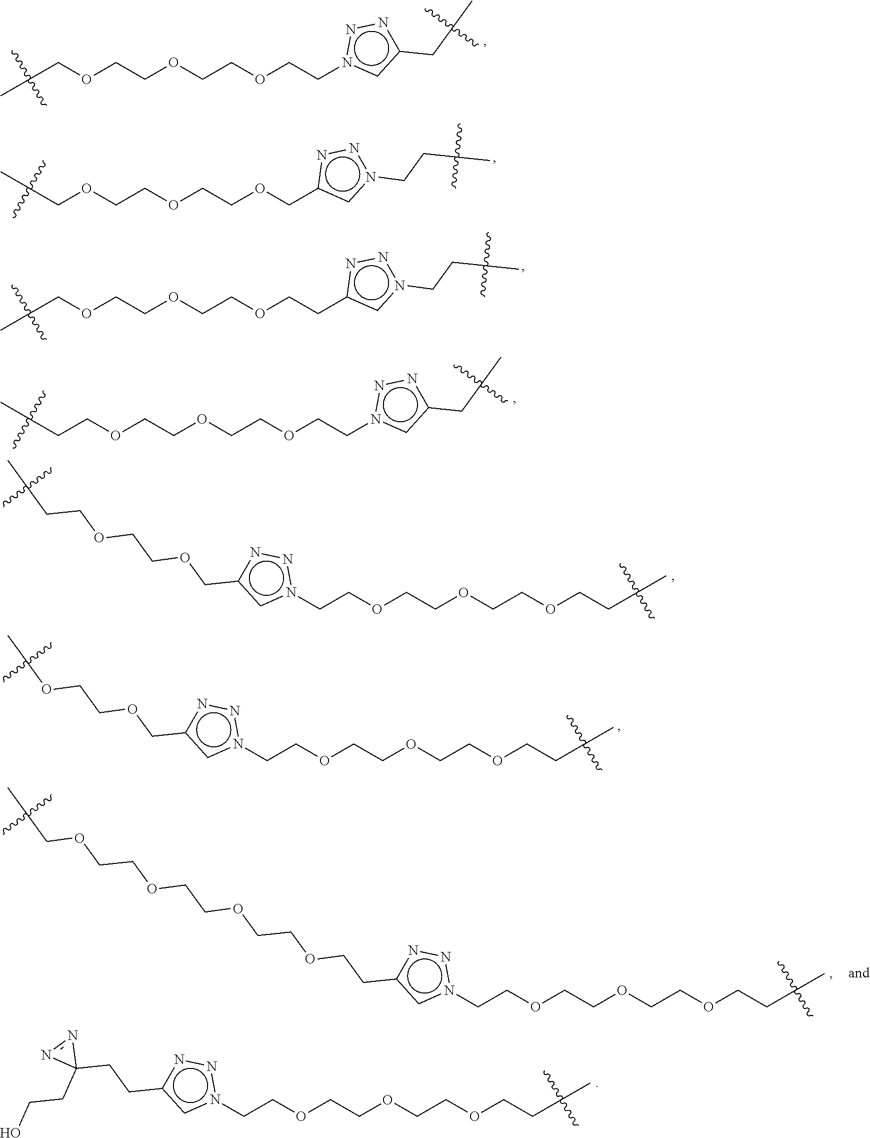

In some embodiments, -L¹- is selected from

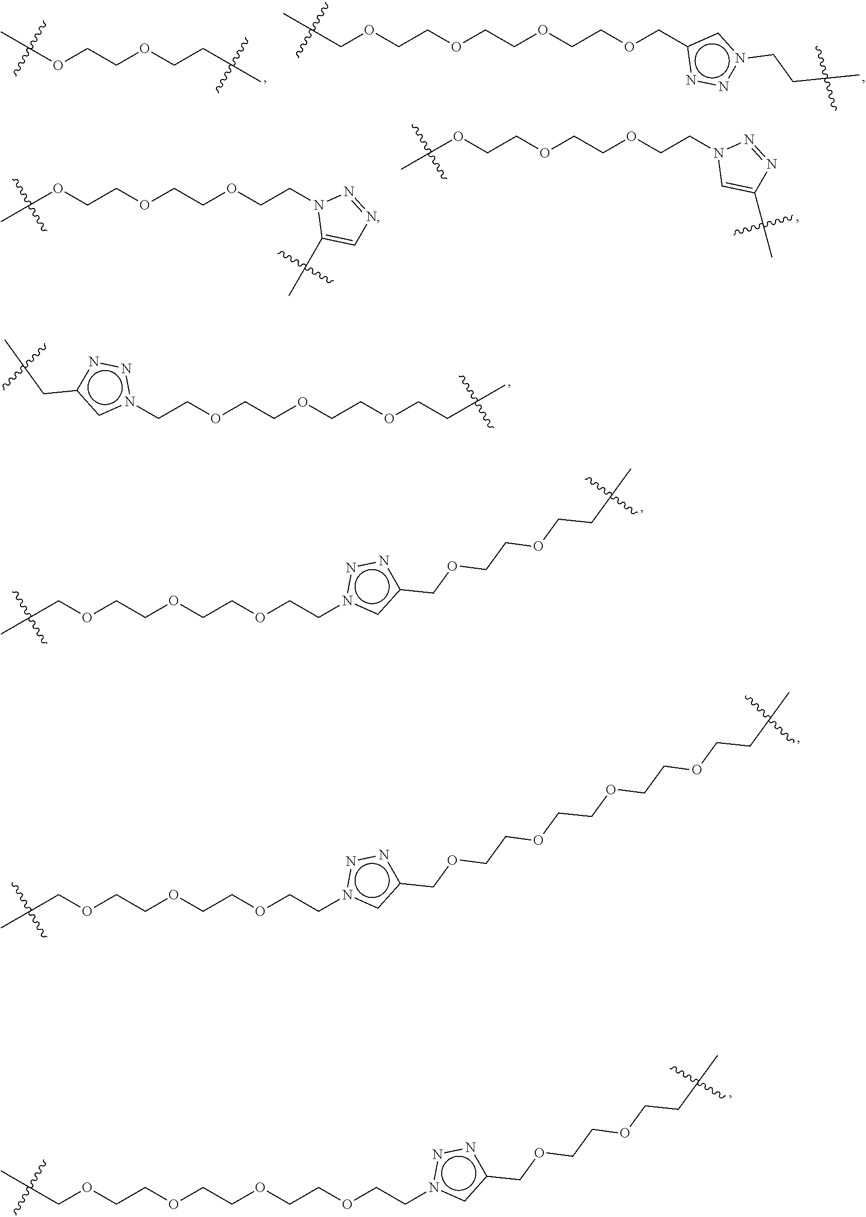

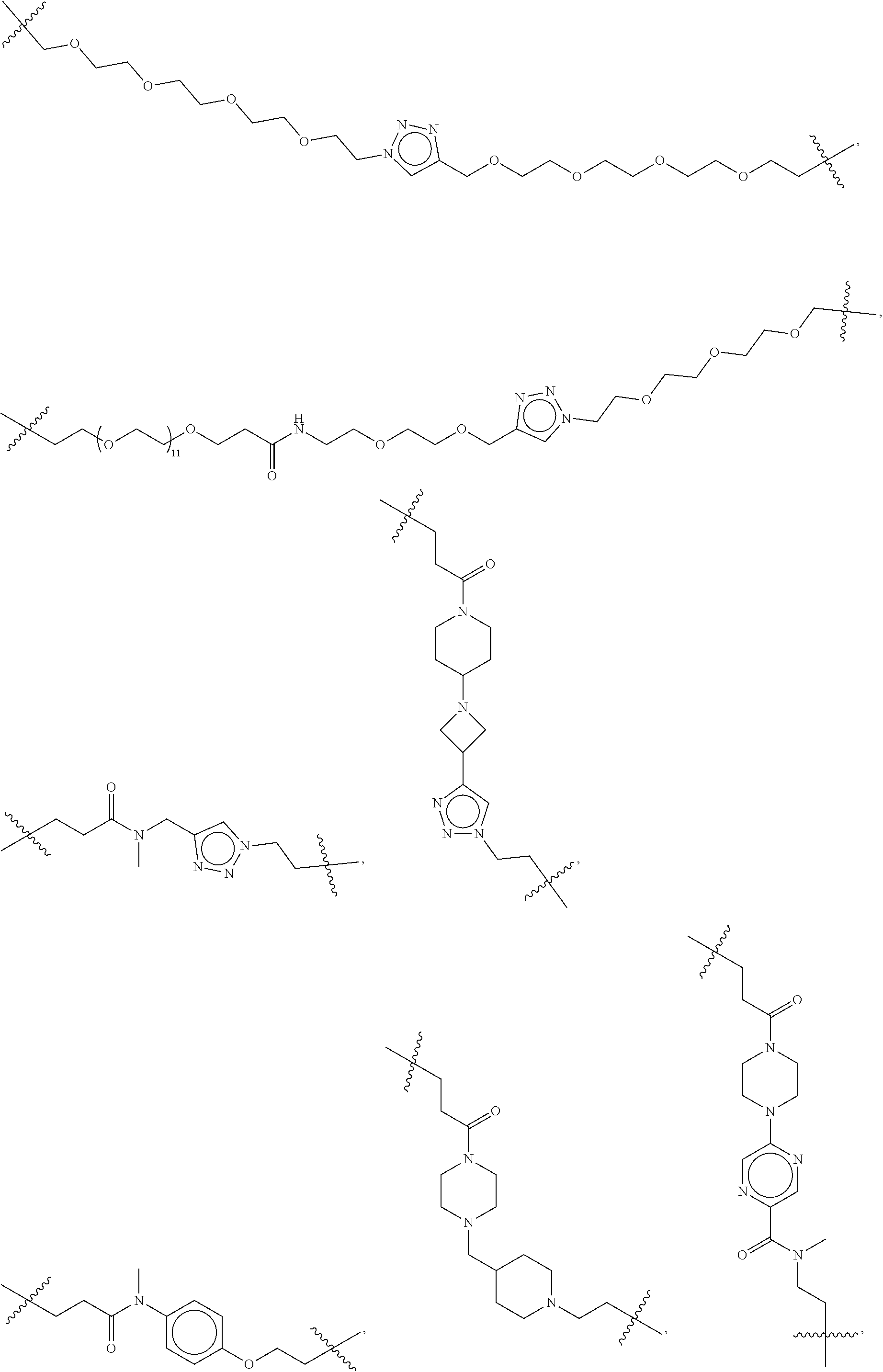

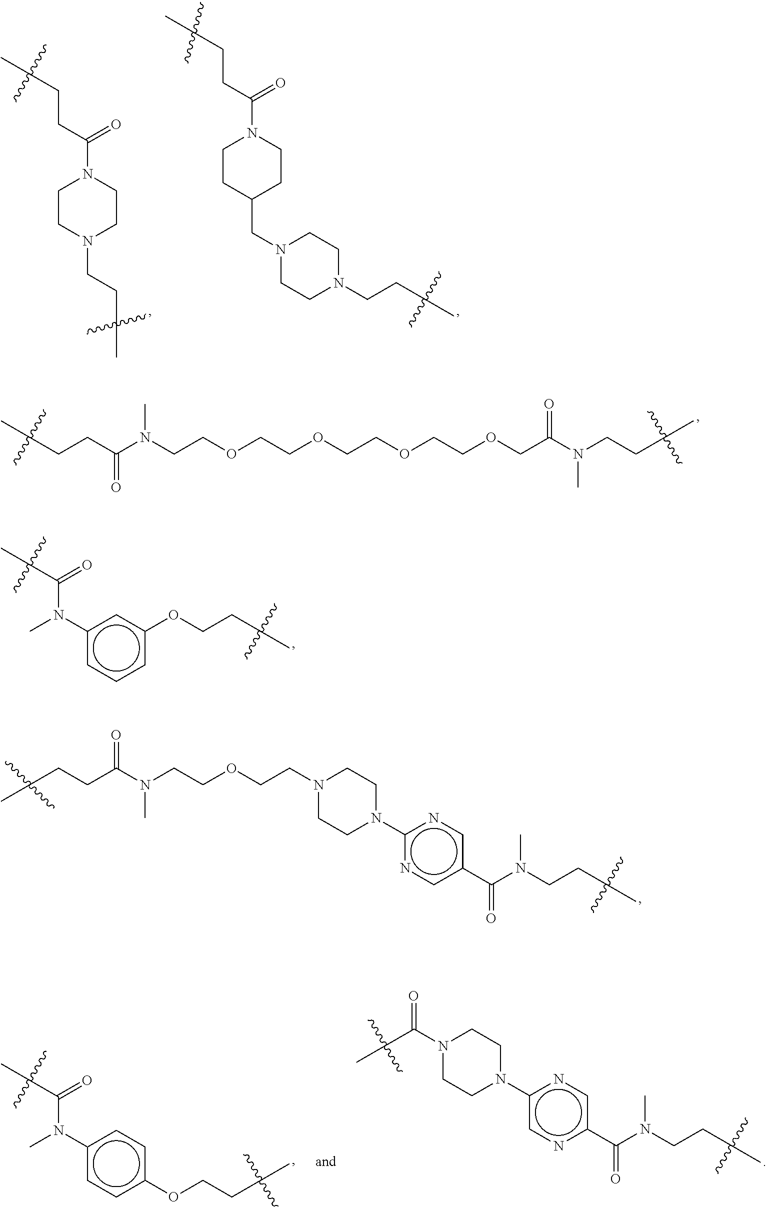

In some embodiments, -L¹- and is selected from

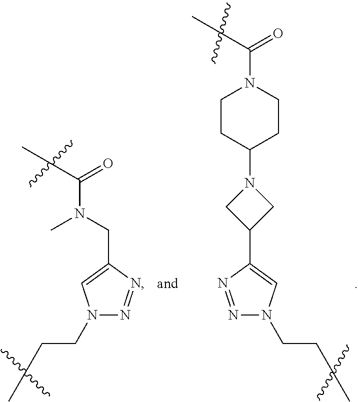 [SMILES: *CCn1cc(CN(C)C(*)=O)nn1 ; *CCn1cc(C2CN(C3CCN(C(*)=O)CC3)C2)nn1]

In some embodiments, R⁸ is -L¹- and is selected from one of those in Table 3, below.

In some embodiments, two R⁸ taken together with the carbon atom to which they are attached, form a cyclopropane ring.

In some embodiments, two R⁸ taken together with the carbon atom to which they are attached, form a cyclobutane ring.

In some embodiments, R⁸ is selected from those depicted in Table 1, below.

In some embodiments, R⁸ is selected from those depicted in Table 1A, below.

In some embodiments, R⁸ is selected from those depicted in Table 1B, below.

In some embodiments, R⁸ is selected from those depicted in Table 1C, below.

As defined generally above, R⁹ is —R, halogen, —CN, —NC, —C(O)OR, —OC(O)R, —OR, —N(R)₂, or —SR.

In some embodiments, R⁹ is R. In some embodiments, R⁹ is halogen. In some embodiments, R⁹ is —CN. In some embodiments, R⁹ is —NC. In some embodiments, R⁹ is —C(O)OR. In some embodiments, R⁹ is —OC(O)R. In some embodiments, R⁹ is —OR. In some embodiments, R⁹ is —N(R)₂. In some embodiments, R⁹ is —SR.

In some embodiments, R⁹ is hydrogen. In some embodiments, R⁹ is an optionally substituted C₁₋₆ aliphatic group. In some embodiments, R⁹ is an optionally substituted 3-8 membered saturated or partially unsaturated monocyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 4-8 membered saturated or partially unsaturated monocyclic heterocyclic ring having 1-2 heteroatoms independently selected from nitrogen, oxygen, and sulfur. In some embodiments, R⁹ is an optionally substituted 5-6 membered monocyclic heteroaromatic ring having 1-4 heteroatoms independently selected from nitrogen, oxygen, and sulfur. In some embodiments, R⁹ is an optionally substituted 8-10 membered bicyclic heteroaromatic ring having 1-5 heteroatoms independently selected from nitrogen, oxygen, and sulfur.

In some embodiments, R⁹ is selected from H, Me, Et, iPr,

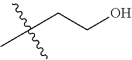 [SMILES: *CCO]

### and OH.

In some embodiments, R⁹ is selected from those depicted in Table 1, below.

In some embodiments, R⁹ is selected from those depicted in Table 1A, below.

In some embodiments, R⁹ is selected from those depicted in Table 1B, below.

In some embodiments, R⁹ is selected from those depicted in Table 1C, below.

In some embodiments, R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur.

As defined generally above, R¹⁰ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹⁰ is R. In some embodiments, R¹⁰ is halogen. In some embodiments, R¹⁰ is —CN. In some embodiments, R¹⁰ is —NC. In some embodiments, R¹⁰ is —C(O)OR. In some embodiments, R¹⁰ is —OC(O)R. In some embodiments, R¹⁰ is —C(O)N(R)₂. In some embodiments, R¹⁰ is —N(R)C(O)R. In some embodiments, R¹⁰ is —N(R)C(O)N(R)₂. In some embodiments, R¹⁰ is —OC(O)N(R)₂. In some embodiments, R¹⁰ is —N(R)C(O)OR. In some embodiments, R¹⁰ is —OR. In some embodiments, R¹⁰ is —N(R)₂. In some embodiments, R¹⁰ is —NO₂. In some embodiments, R¹⁰ is —N₃. In some embodiments, R¹⁰ is —SR. In some embodiments, R¹⁰ is —S(O)R. In some embodiments, R¹⁰ is —S(O)₂R. In some embodiments, R¹⁰ is —S(O)₂N(R)₂. In some embodiments, R¹⁰ is —NRS(O)₂R.

In some embodiments, R¹⁰ is hydrogen. In some embodiments, R¹⁰ is an optionally substituted C₁₋₆ aliphatic group. In some embodiments, R¹⁰ is an optionally substituted 3-8 membered saturated or partially unsaturated monocyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 4-8 membered saturated or partially unsaturated monocyclic heterocyclic ring having 1-2 heteroatoms independently selected from nitrogen, oxygen, and sulfur. In some embodiments, R¹⁰ is an optionally substituted 5-6 membered monocyclic heteroaromatic ring having 1-4 heteroatoms independently selected from nitrogen, oxygen, and sulfur. In some embodiments, R¹⁰ is an optionally substituted 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring. In some embodiments, one instance of —C(R¹⁰)₂— is optionally replaced by phenyl. In some embodiments, one instance of —C(R¹⁰)₂— is optionally replaced by an 8-10 membered bicyclic aromatic carbocyclic ring. In some embodiments, one instance of —C(R¹⁰)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R¹⁰)₂— is optionally replaced by a 5-6 membered monocyclic heteroaromatic ring having 1-4 heteroatoms independently selected from nitrogen, oxygen, and sulfur. In some embodiments, one instance of —C(R¹⁰)₂— is optionally replaced by an 8-10 membered bicyclic heteroaromatic ring having 1-5 heteroatoms independently selected from nitrogen, oxygen, and sulfur.

In some embodiments, one instance of —C(R¹⁰)₂— is optionally replaced by

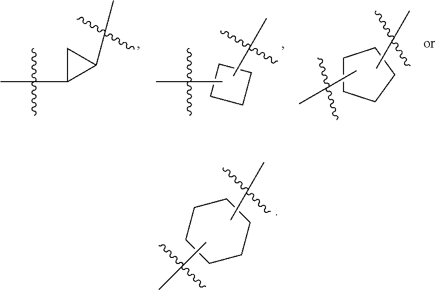

In some embodiments, two R¹⁰ taken together with the carbon atom to which they are attached, form a cyclopropane ring.

In some embodiments, R¹⁰ is selected from H, Me, Et, iPr,

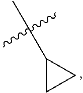 [SMILES: *C1CC1]

### CF₃,

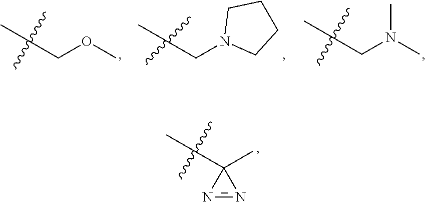

### and CH₃OH.

In some embodiments, R¹⁰ is -L¹- and is selected from

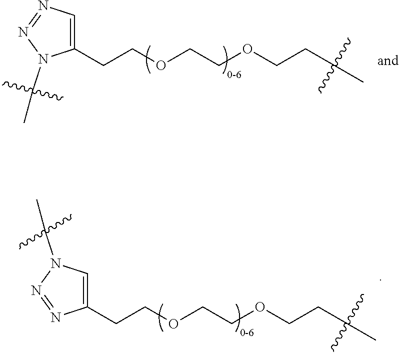

In some embodiments, R¹⁰ is -L¹- and is selected from

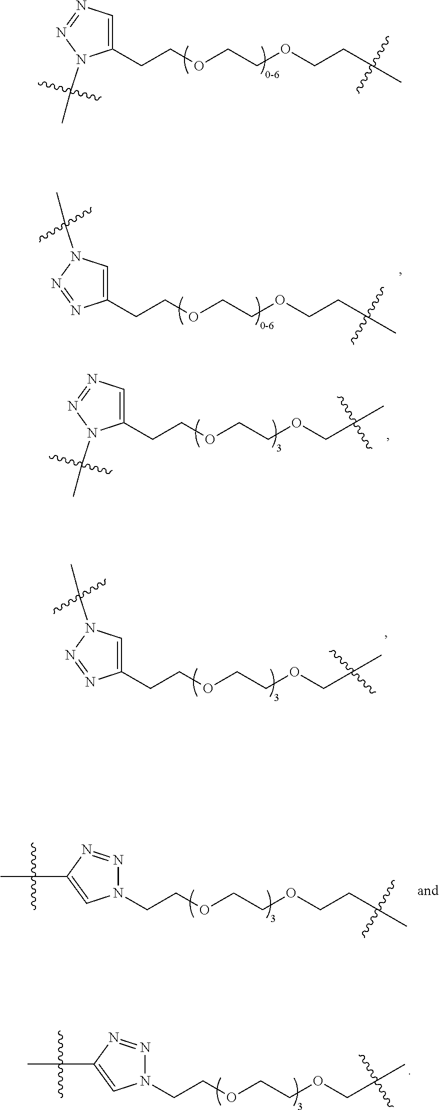

In some embodiments, R¹⁰ is -L¹- and is selected from

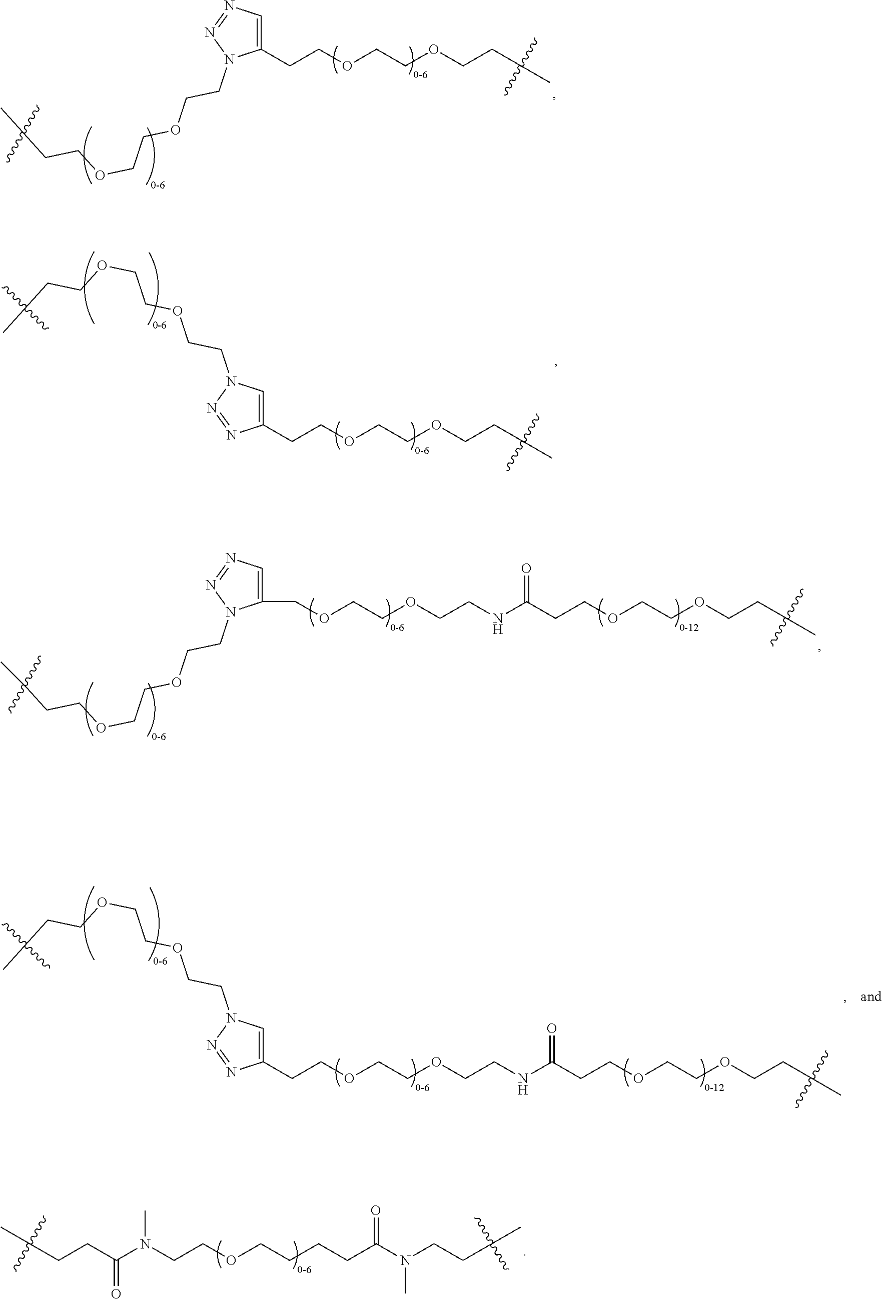

In some embodiments R¹⁰ is -L¹- and is selected from

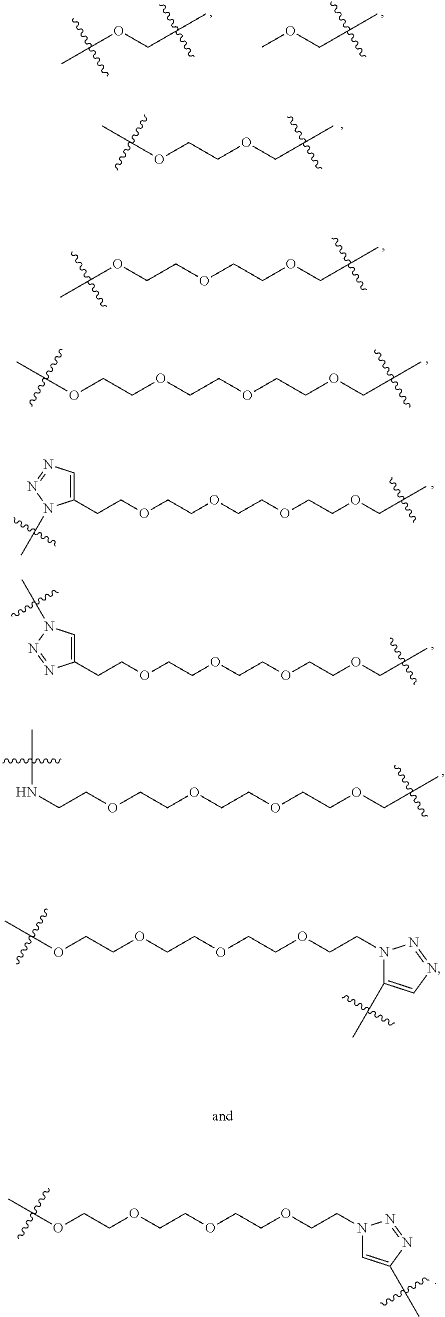

In some embodiments, R¹⁰ is -L¹- and is selected from

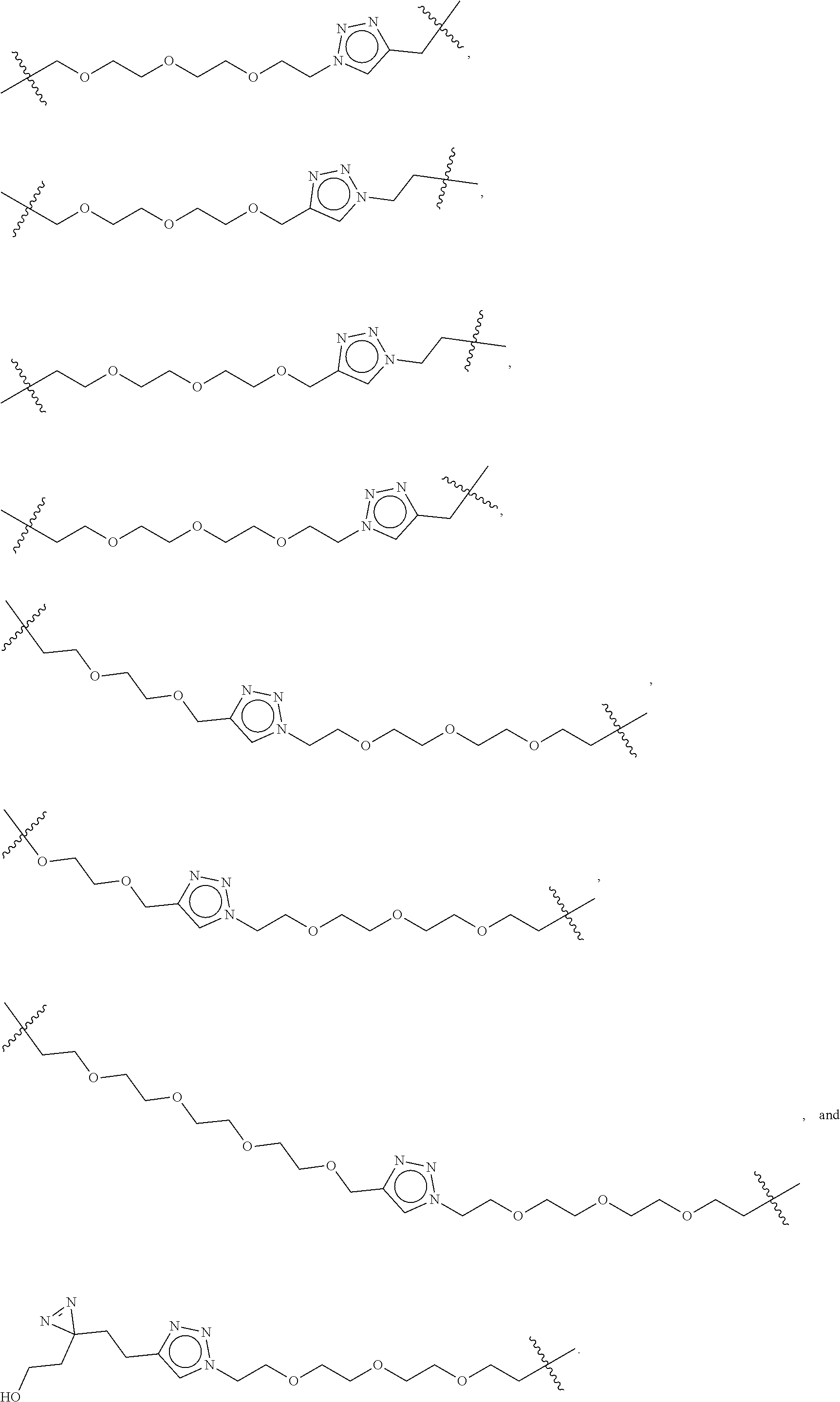

In some embodiments, R¹⁰ is -L¹- and is selected from

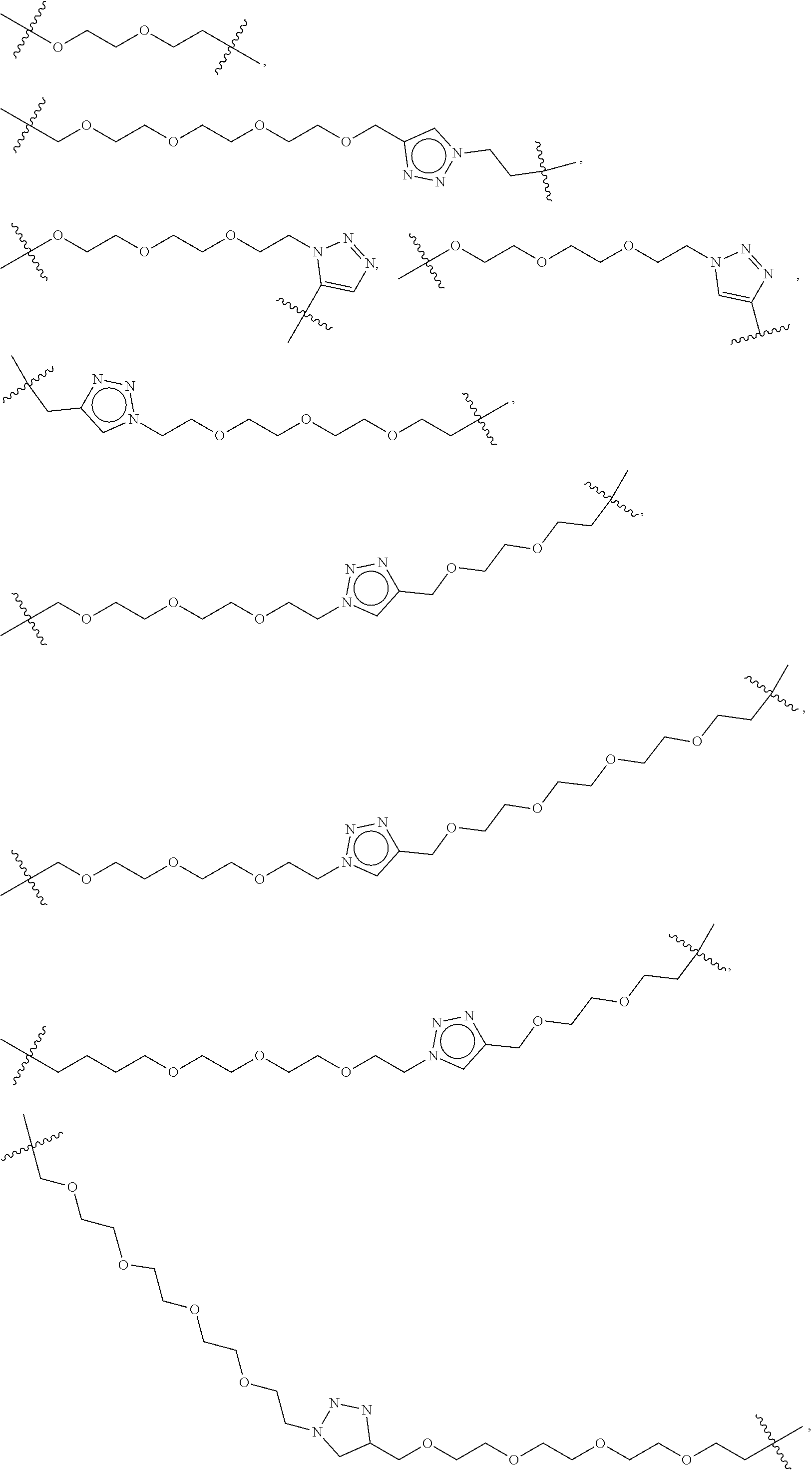

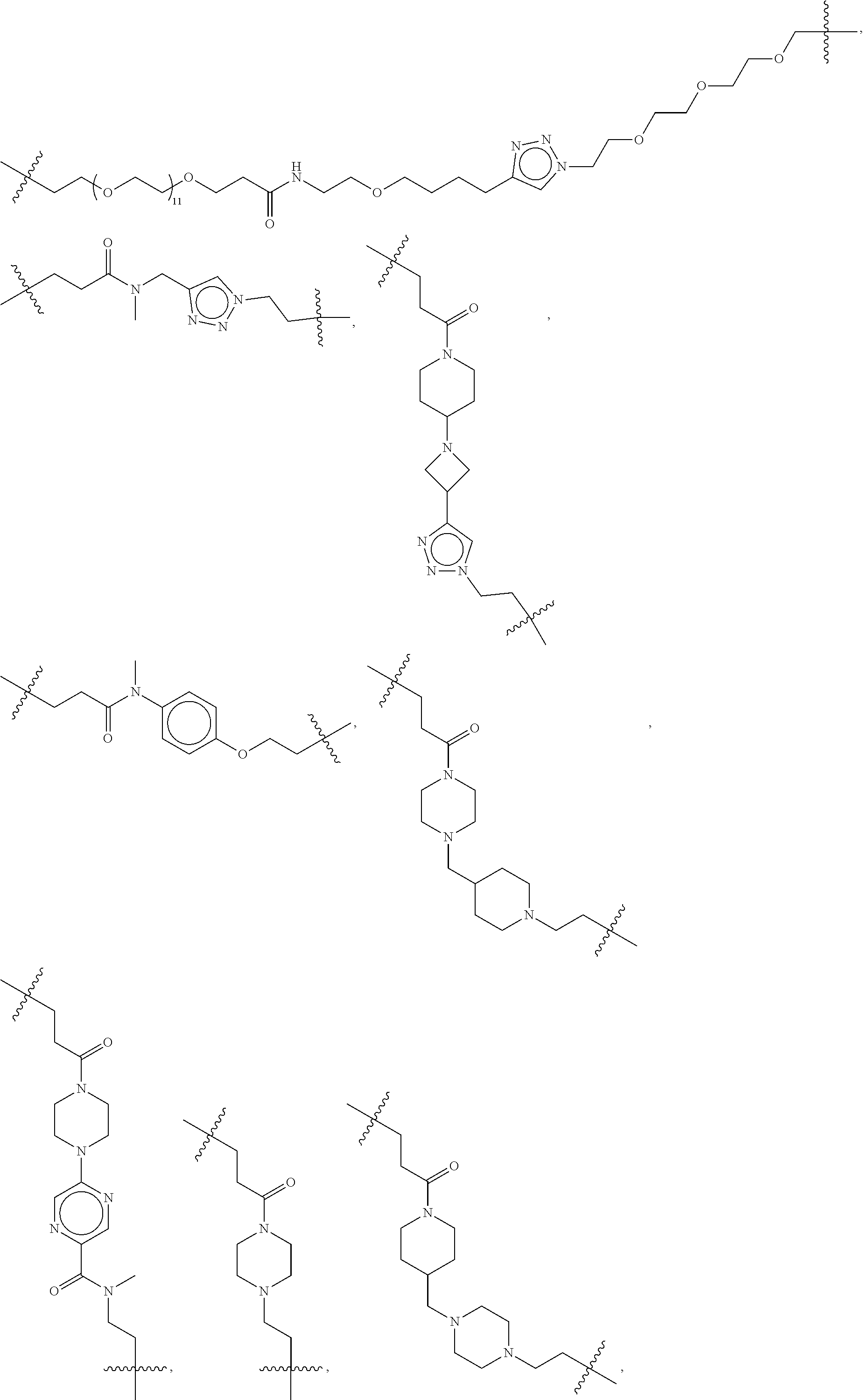

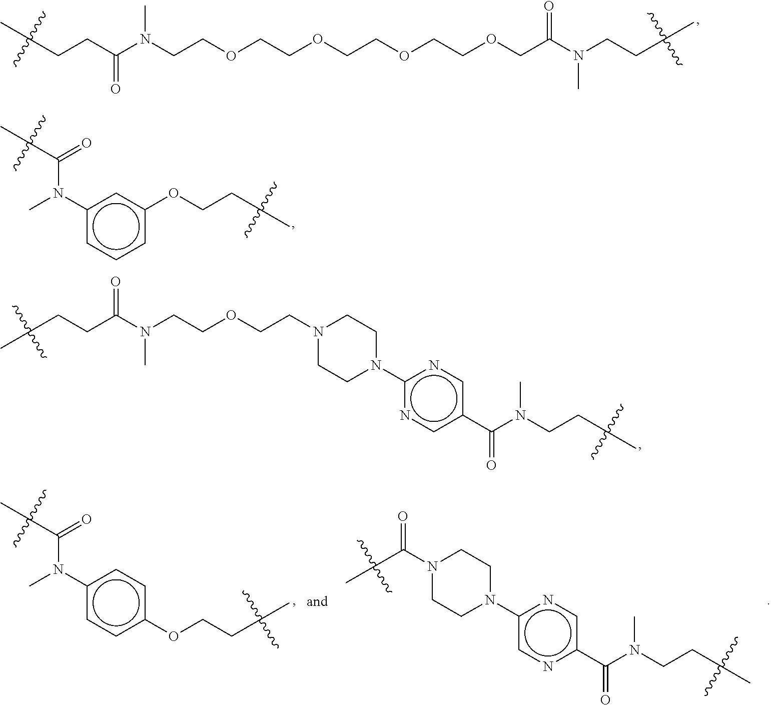

In some embodiments, R¹⁰ is -L¹- and is selected from one of those in Table 3, below.

In some embodiments, R¹⁰ is selected from those depicted in Table 1, below.

In some embodiments, R¹⁰ is selected from those depicted in Table 1A, below.

In some embodiments, R¹⁰ is selected from those depicted in Table 1B, below.

In some embodiments, R¹⁰ is selected from those depicted in Table 1C, below.

In some embodiments, R⁸ and R¹⁰ taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur.

In some embodiments, R⁹ and R¹⁰ taken together with the atoms to which they are attached, form a ring substituted with m instances of R⁵; wherein the ring is a 4-8 membered saturated or partially unsaturated monocyclic heterocyclic ring having 1-2 heteroatoms independently selected from nitrogen, oxygen, and sulfur.

As defined generally above, -L¹- is a bivalent linker group that covalently connects the RNA Binder to the DFL; wherein one and only one of R¹, R², R³, R⁸, or R¹¹ is -L¹- and one end of -L¹- is covalently bound to rSM.

In some embodiments, -L¹- is defined in an embodiment described in the section entitled “Linkers” below.

In some embodiments, -L¹- is a covalent bond or a C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-, and 1, 2, 3, 4, 5, 6, or 7 methylene units are optionally replaced with —OCH₂CH₂— or —CH₂CH₂O—; wherein one and only one of R¹, R², R³, R⁸, or R¹¹ is -L¹- and one end of -L¹- is covalently bound to rSM.

In some embodiments, -L¹- is a covalent bond or a C₁₋₁₆ bivalent straight or branched hydrocarbon chain wherein 1, 2, 3, 4, 5 or 6 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-, and 1, 2, 3, 4, 5, 6, or 7 methylene units are optionally replaced with —OCH₂CH₂— or —CH₂CH₂O—; wherein one and only one of R¹, R², R³, R⁸, or R¹¹ is -L¹- and one end of -L¹- is covalently bound to rSM.

As defined generally above, R¹¹ is H, C₁₋₃ alkyl, or -L¹-.

In some embodiments, R¹¹ is H. In some embodiments, R¹¹ is C₁₋₃ alkyl. In some embodiments, R¹¹ is -L¹-.

In some embodiments, R¹¹ is -L¹- and is selected from

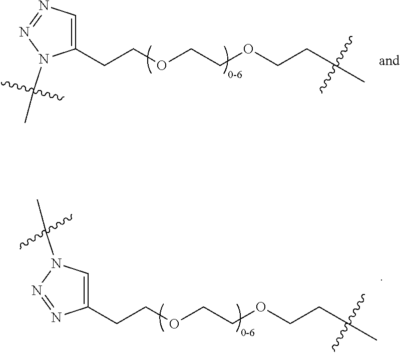

In some embodiments, R¹¹ is -L¹- and is selected from

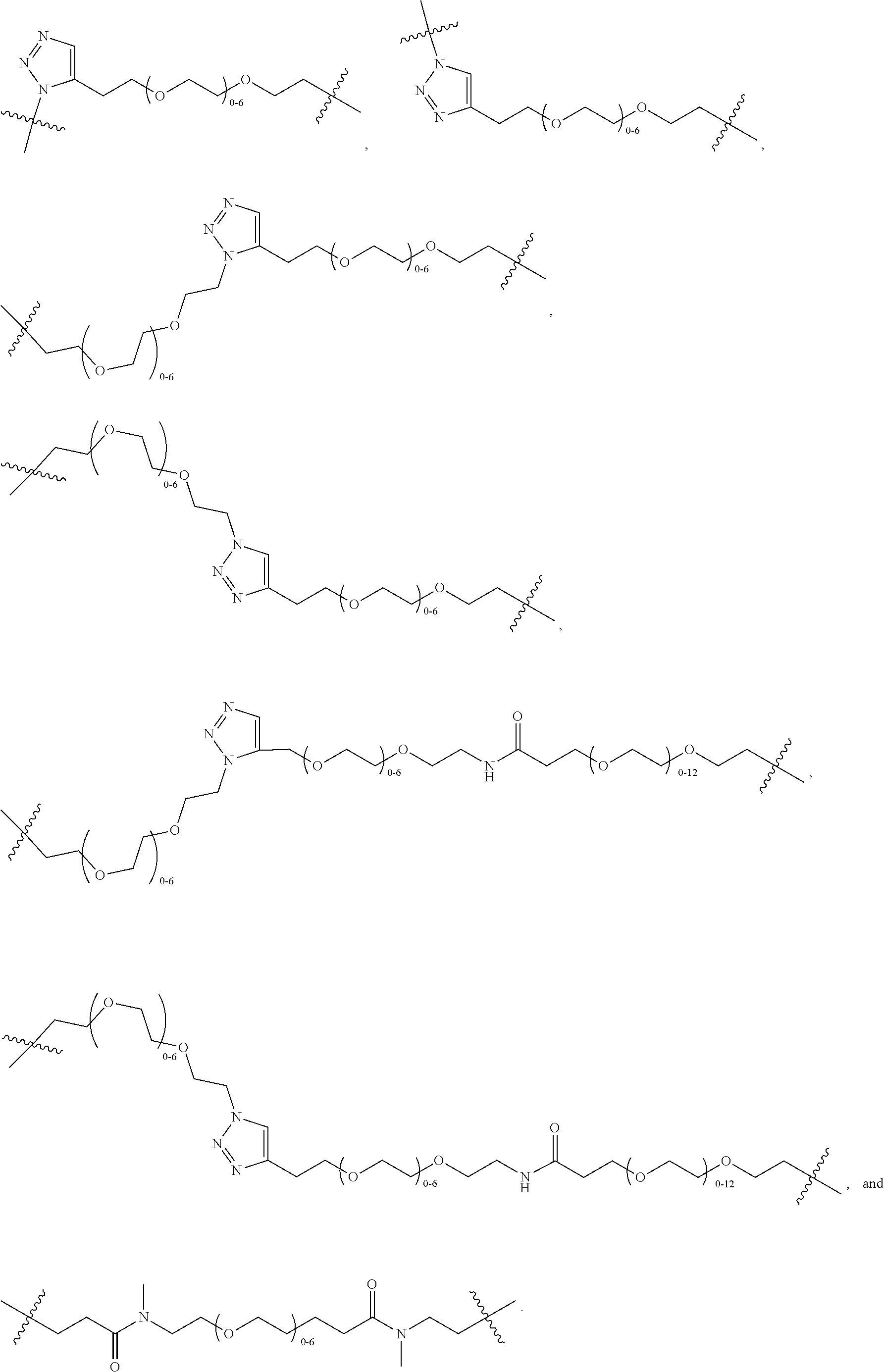

In some embodiments R¹ is -L¹- and is selected from

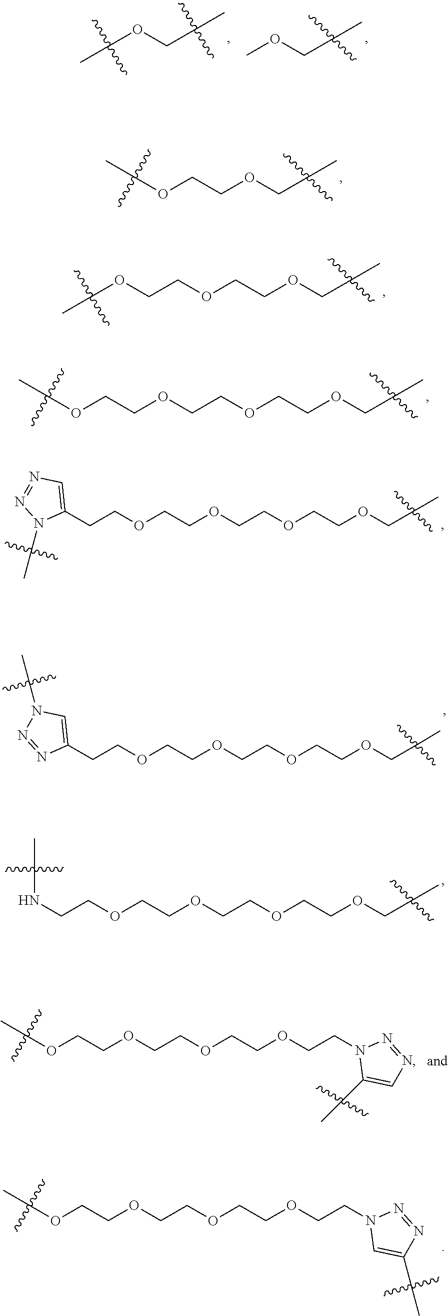

In some embodiments, R¹¹ is -L¹- and is selected from

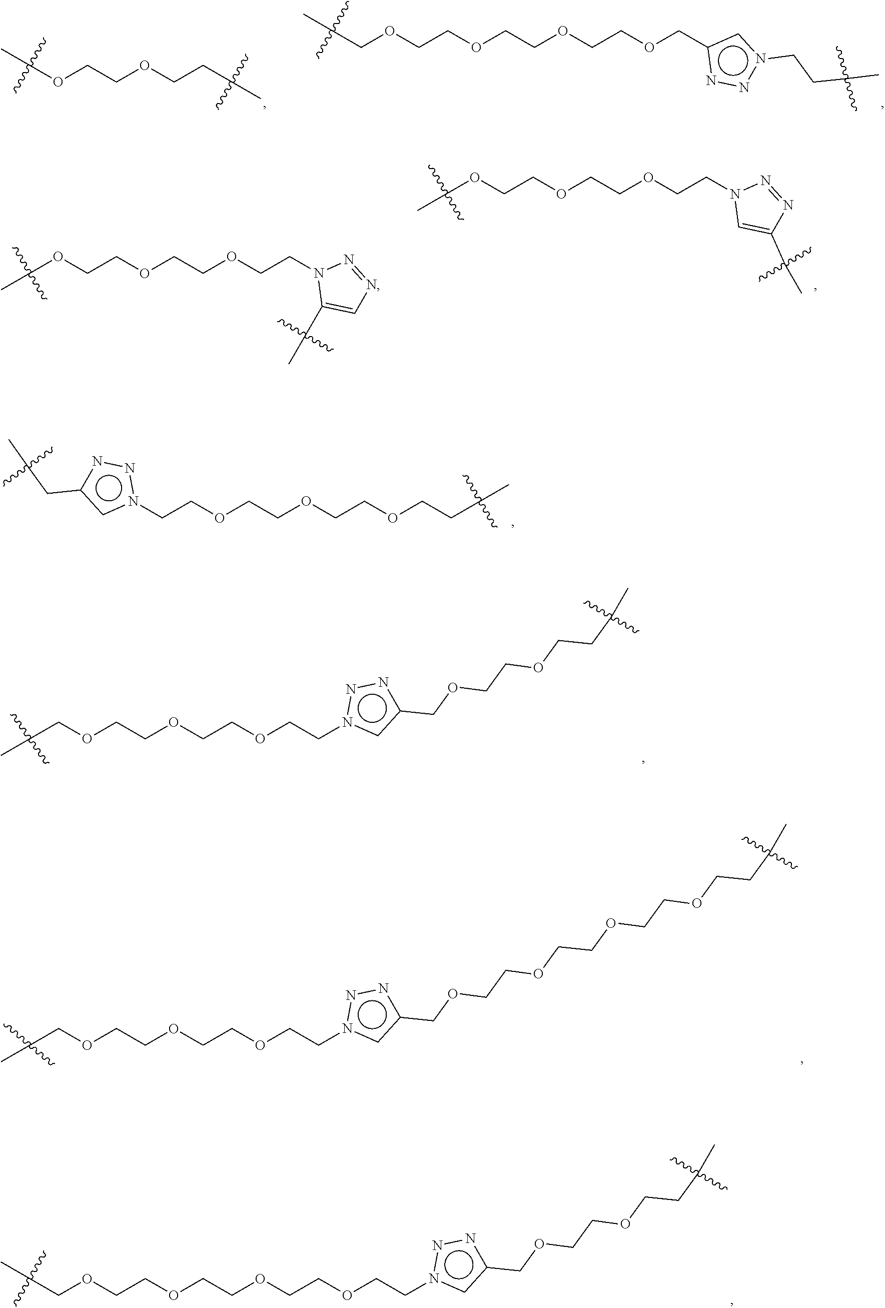

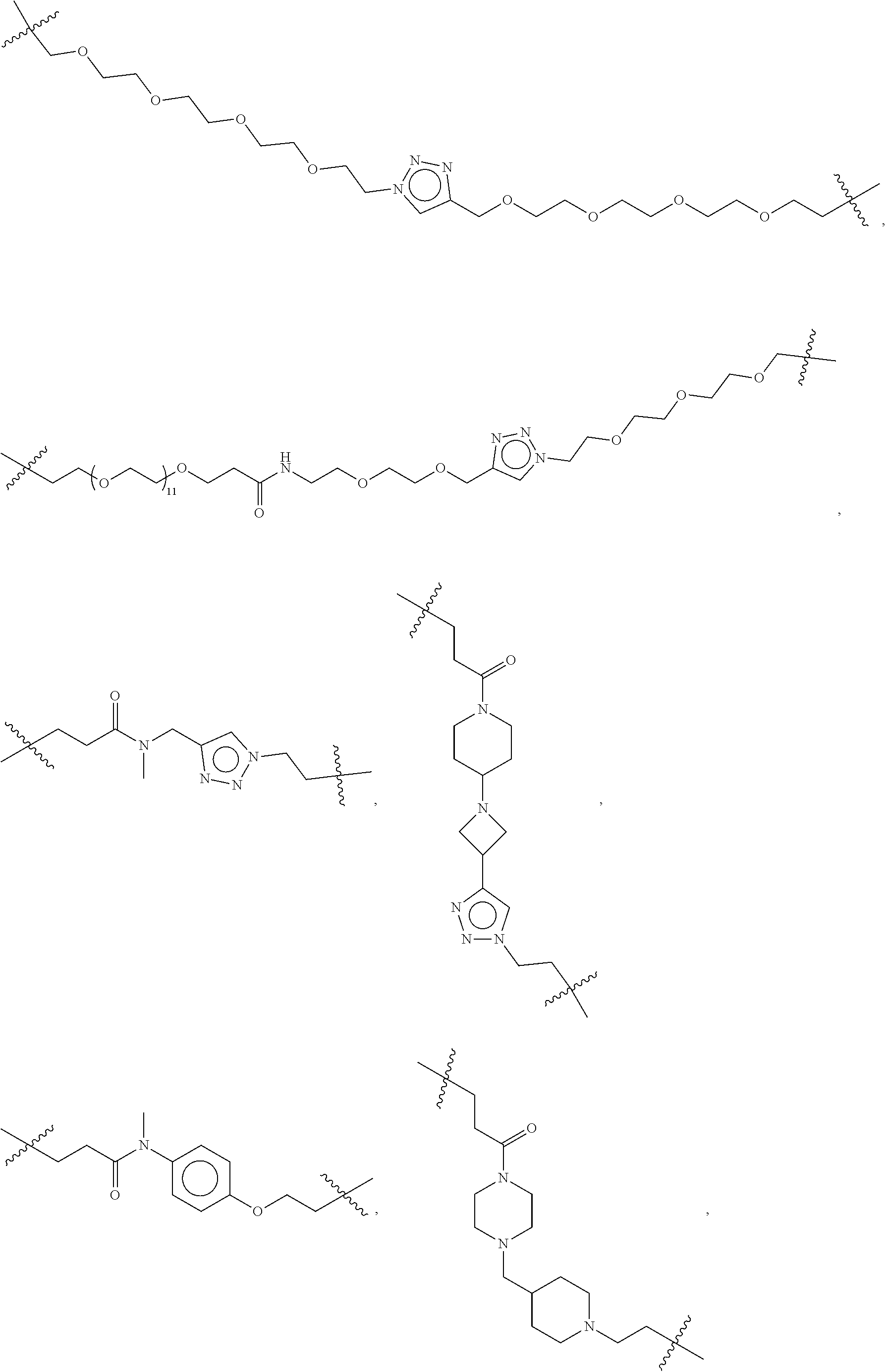

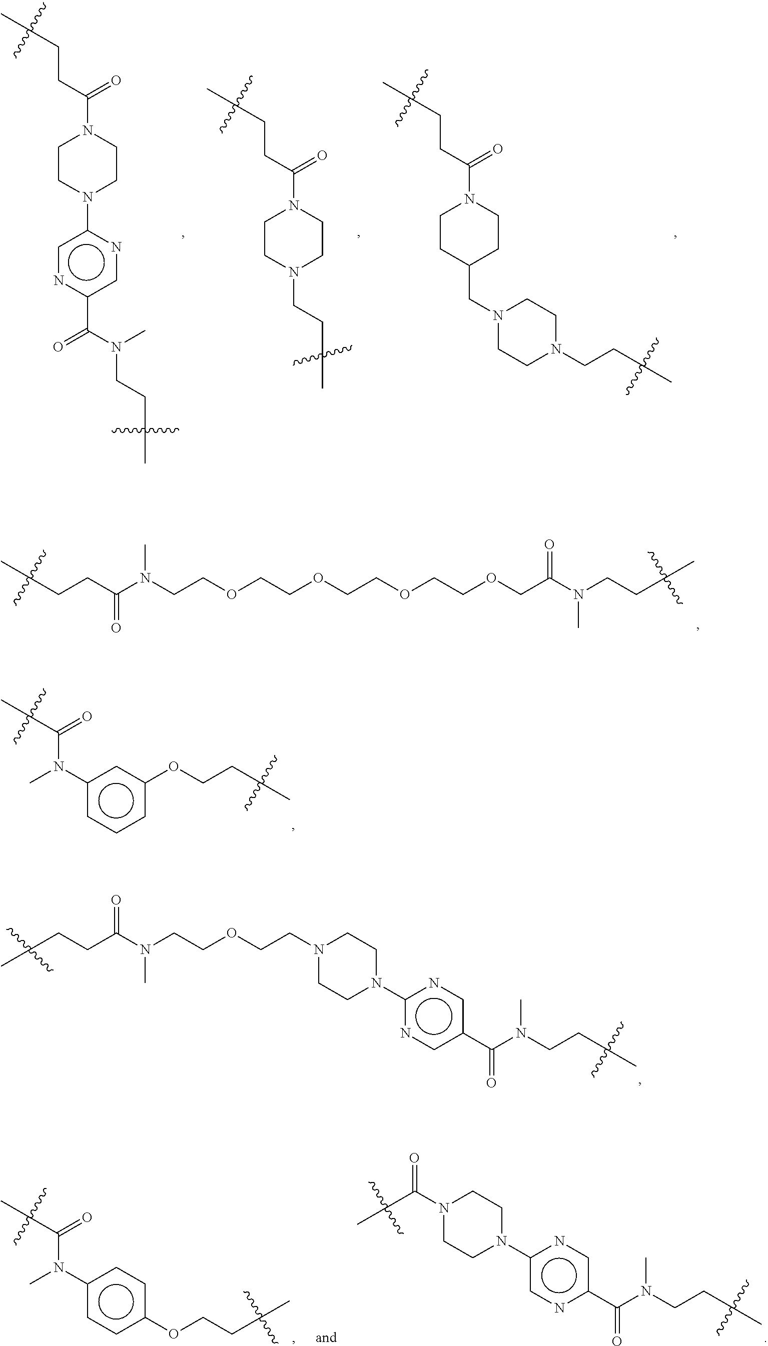

In some embodiments, R¹¹ is -L¹- and is selected from one of those in Table 3, below.

In some embodiments, R¹¹ is selected from those depicted in Table 1A, below.

In some embodiments, R¹¹ is selected from those depicted in Table 1B, below.

In some embodiments, R¹¹ is selected from those depicted in Table 1C, below.

In some embodiments, -L²- is selected from

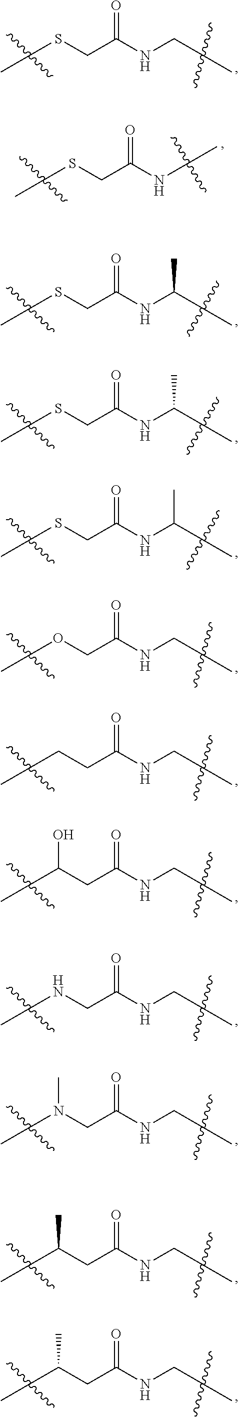

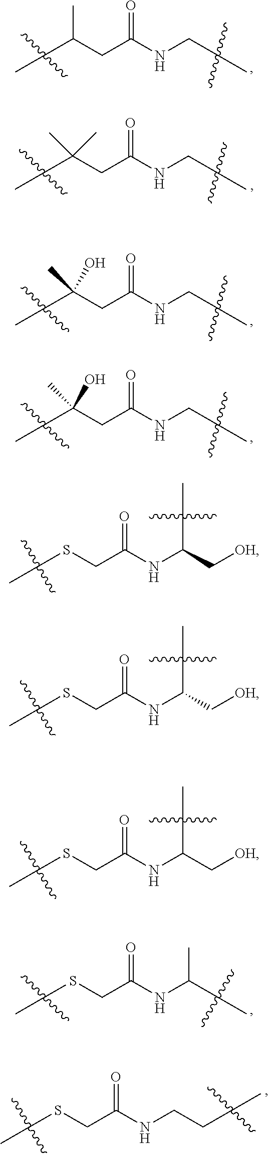

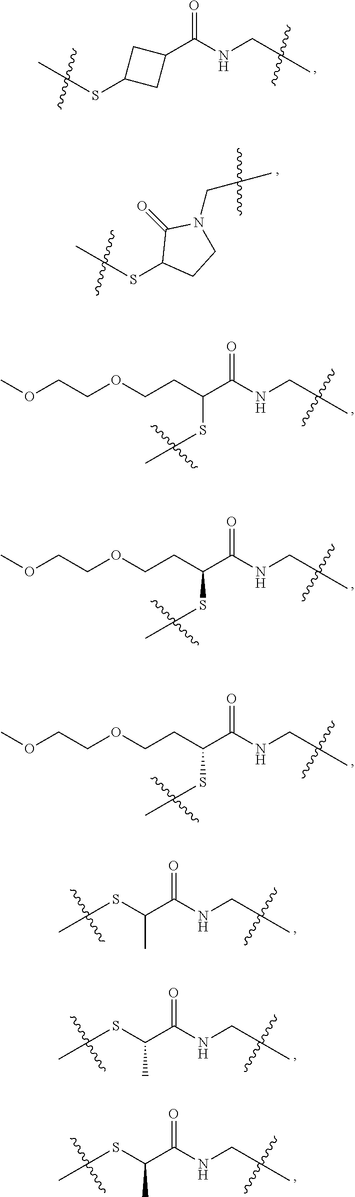

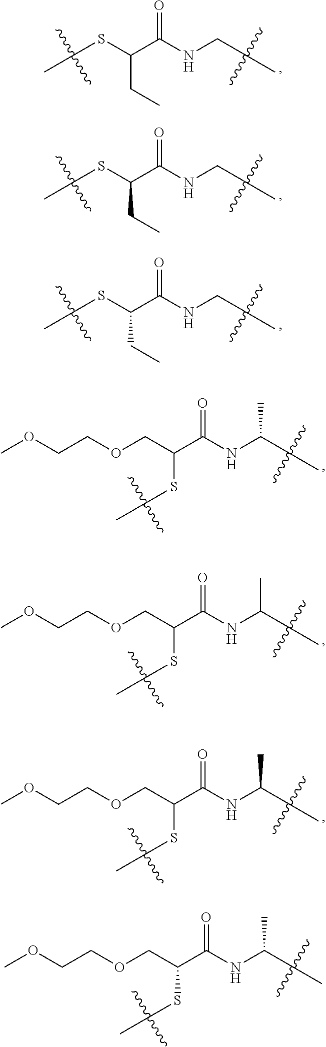

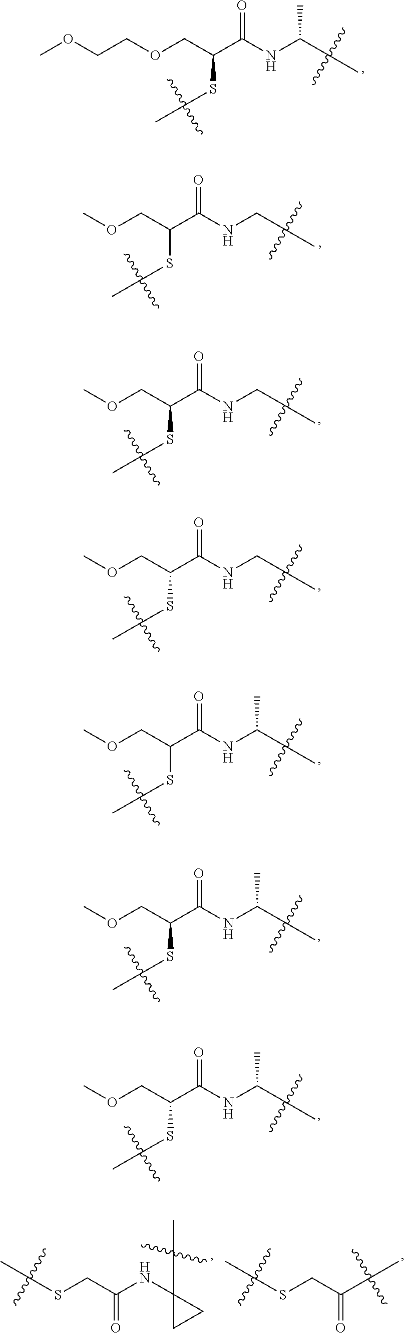

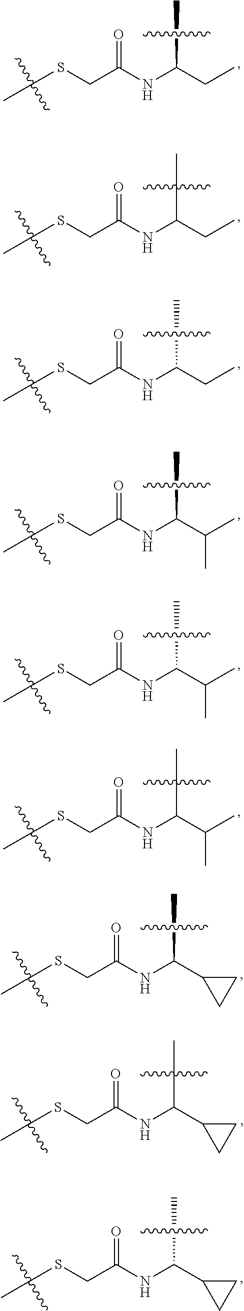

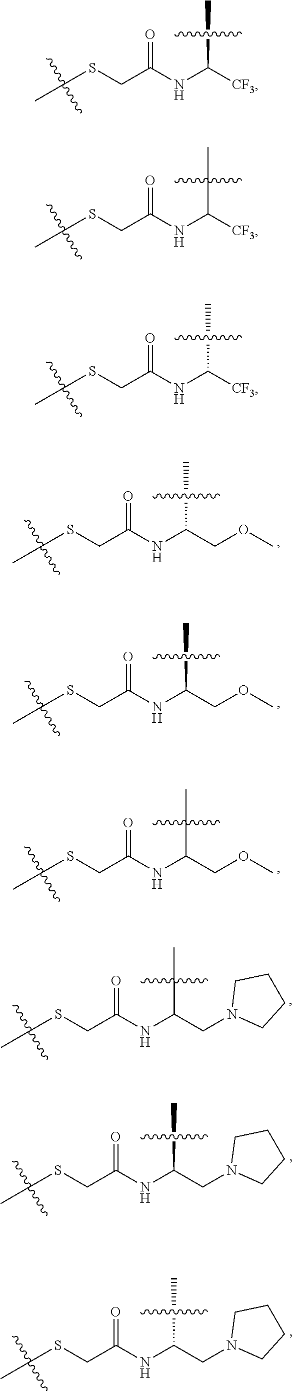

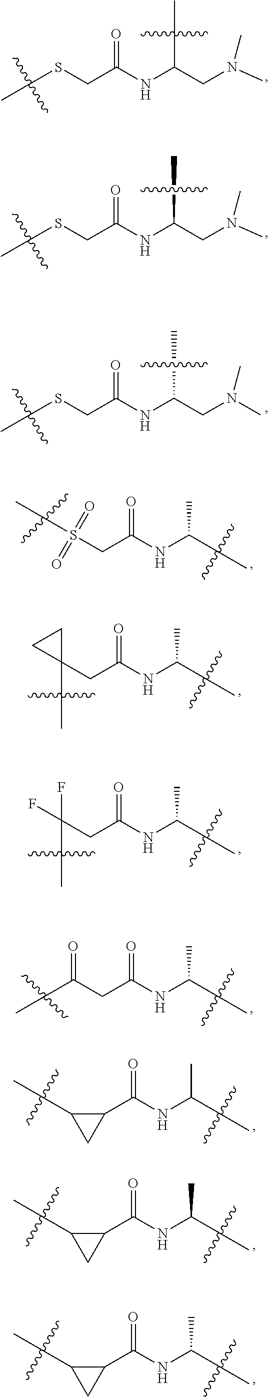

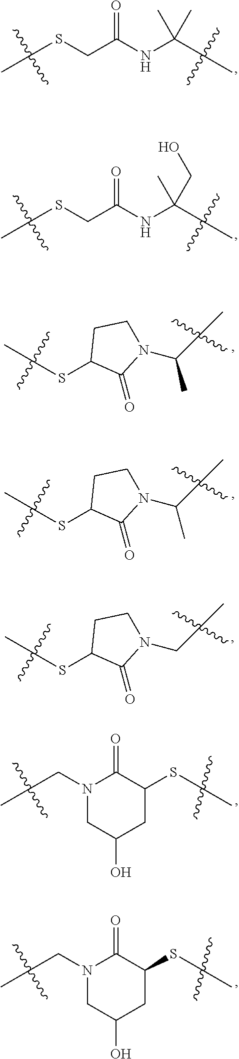

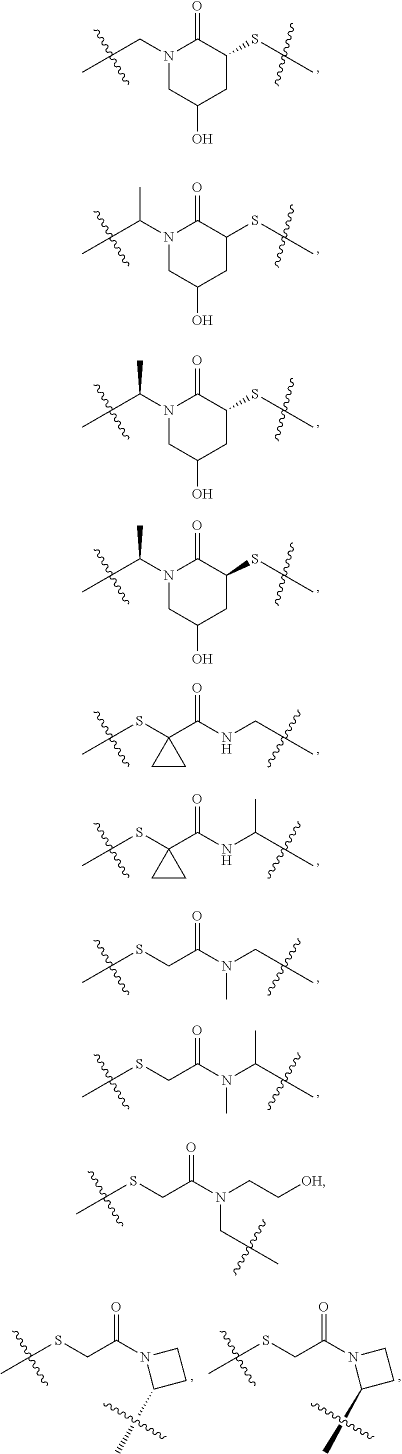

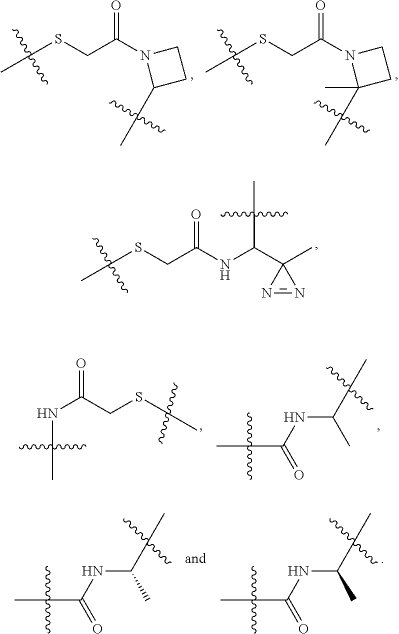

In some embodiments, -L²- is selected from

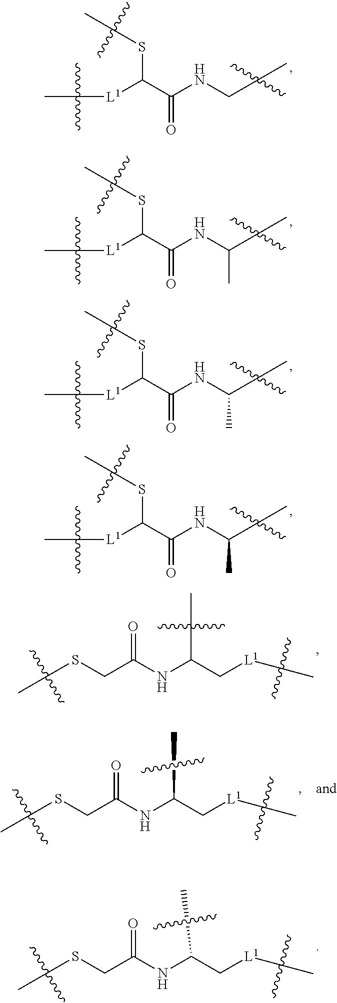

In some embodiments, -L²- is selected from those depicted in Table 1, below.

In some embodiments, -L²- is selected from those depicted in Table 1A, below.

In some embodiments, -L²- is selected from those depicted in Table 1B, below.

In some embodiments, -L²- is selected from those depicted in Table 1C, below.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0, 1, 2, or 3. In some embodiments, m is 0, 1, or 2. In some embodiments, m is 1, 2, or 3.

As defined generally above, n is 0, 1, 2, 3, 4 or 5. In some embodiments, n is 0. In some embodiments, n is 1. In some embodiments, n is 2. In some embodiments, n is 3. In some embodiments, n is 4. In some embodiments, n is 5. In some embodiments, n is 0, 1, 2, 3 or 4. In some embodiments, n is 0, 1, 2, or 3. In some embodiments, n is 0, 1, or 2. In some embodiments,
n is 1, 2, or 3.

As defined generally above, p is 0, 1, 2, or 3. In some embodiments, p is 0. In some embodiments, p is 1. In some embodiments, p is 2. In some embodiments, p is 3. In some embodiments, p is 0, 1, 2, or 3. In some embodiments, p is 0, 1, or 2. In some embodiments, p is 1, 2, or 3.

As defined generally above, q is 0, 1, 2, 3, or 4. In some embodiments, q is 0. In some embodiments, q is 1. In some embodiments, q is 2. In some embodiments, q is 3. In some embodiments, q is 4. In some embodiments, q is 0, 1, 2, or 3. In some embodiments, q is 0, 1, or 2. In some embodiments, q is 1, 2, or 3.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0, 1, 2, or 3. In some embodiments, r is 0, 1, or 2. In some embodiments, r is 1, 2, or 3.

Exemplary

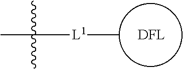

compounds of the invention are set forth in Table 1, below.

Exemplary

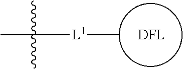

compounds of the invention are set forth in Table 1A, below.

Exemplary

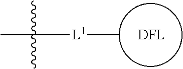

compounds of the invention are set forth in Table 1B, below.

Exemplary

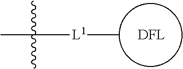

compounds of the invention are set forth in Table 1C, below.

Further exemplary compounds of the invention are set forth below, wherein the

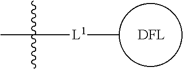

portion of the compound of Formula A or B is depicted in the following formulae.

In some embodiments, the present invention provides a compound of Formula II-a or II-b:

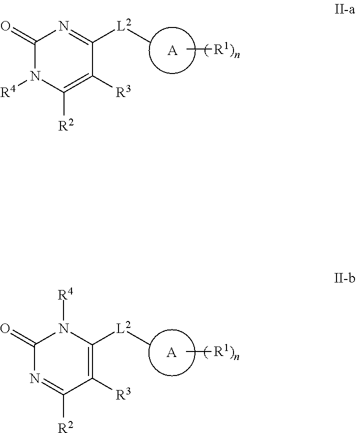

or a pharmaceutically acceptable salt thereof, wherein:
each of Ring A, R, R¹, R², R³, R⁴, R⁶, R⁷, R⁸, R⁹, R¹⁰, -L²-, -L¹-, X, -Cy-, n, q, and r is as defined above and described in embodiments herein, both singly and in combination.

In some embodiments, the present invention provides a compound of Formula III-a:

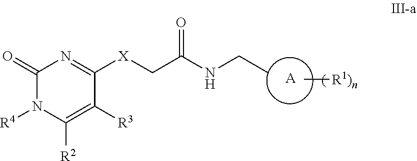

or a pharmaceutically acceptable salt thereof, wherein:
each of Ring A, R, R¹, R², R³, R⁴, R⁶, R⁷, -L¹-, X, -Cy-, and n is as defined above and described in embodiments herein, both singly and in combination.

In some embodiments, the present invention provides a compound of Formula IV-a or IV-b:

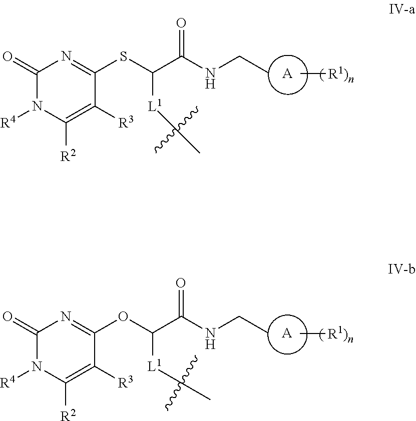

or a pharmaceutically acceptable salt thereof, wherein:
each of Ring A, R, R¹, R², R³, R⁴, -L¹-, -Cy- and n is as defined above and described in embodiments herein, both singly and in combination.

In some embodiments, the present invention provides a compound of Formula IV-c or IV-d:

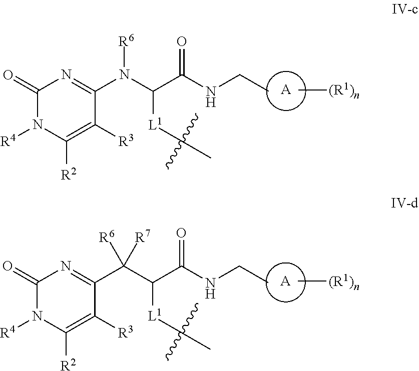

or a pharmaceutically acceptable salt thereof, wherein:
each of Ring A, R, R¹, R², R³, R⁴, R⁶, R⁷, -L¹-, -Cy-, and n is as defined above and described in embodiments herein, both singly and in combination.

In some embodiments, the present invention provides a compound of Formula V-a:

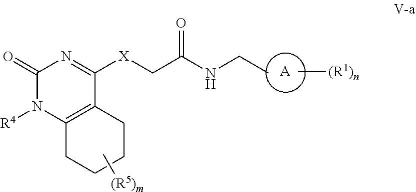

or a pharmaceutically acceptable salt thereof, wherein:
each of Ring A, R, R¹, R⁴, R⁵, R⁶, R⁷, -L¹-, X, -Cy-, m and n is as defined above and described in embodiments herein, both singly and in combination.

In some embodiments, the present invention provides a compound of Formula VI-a or VI-b:

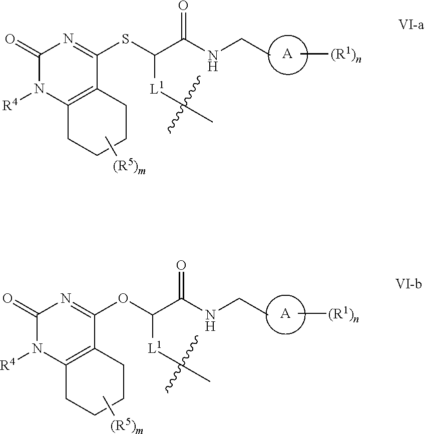

or a pharmaceutically acceptable salt thereof, wherein:
each of Ring A, R, R¹, R², R³, R⁴, R⁵, -L¹-, -Cy-, m and n is as defined above and described in embodiments herein, both singly and in combination.

In some embodiments, the present invention provides a compound of Formula VI-c or VI-d:

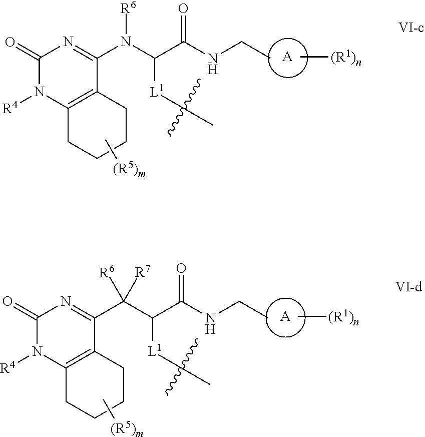

or a pharmaceutically acceptable salt thereof, wherein:
each of Ring A, R, R¹, R², R³, R⁴, R⁵, R⁶, R⁷, -L¹-, -Cy-, m and n is as defined above and described in embodiments herein, both singly and in combination.

In some embodiments, the present invention provides a compound of Formula VII-a:

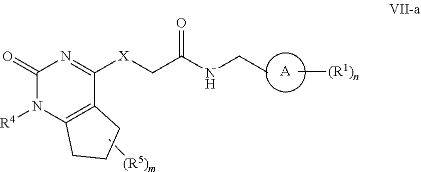

or a pharmaceutically acceptable salt thereof, wherein:
each of Ring A, R, R¹, R⁴, R⁵, R⁶, R⁷, -L¹-, X, -Cy-, m and n is as defined above and described in embodiments herein, both singly and in combination.

In some embodiments, the present invention provides a compound of Formula VIII-a or VIII-b:

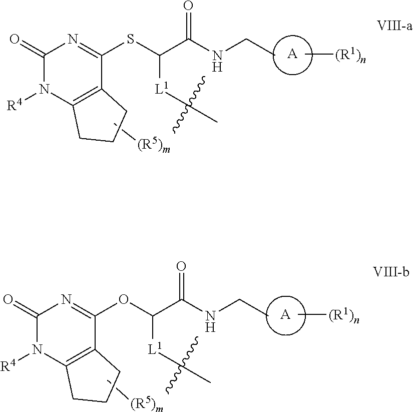

or a pharmaceutically acceptable salt thereof, wherein:
each of Ring A, R, R¹, R², R³, R⁴, R⁵, -L¹-, -Cy-, m and n is as defined above and described in embodiments herein, both singly and in combination.

In some embodiments, the present invention provides a compound of Formula VIII-c or VIII-d:

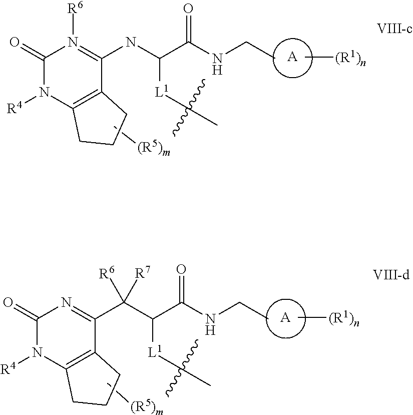

or a pharmaceutically acceptable salt thereof, wherein each of Ring A, R, R¹, R², R³, R⁴, R⁵, R⁶,
R⁷, -L¹-, -Cy-, m and n is as defined above and described in embodiments herein, both singly and in combination.

Further exemplary compounds of the invention are set forth below, wherein the

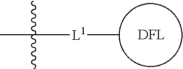

portion of the compound of Formula A or B is depicted in the following formulae.

In some embodiments, the present invention provides a compound of Formula IX-a, Formula IX-b or Formula IX-c:

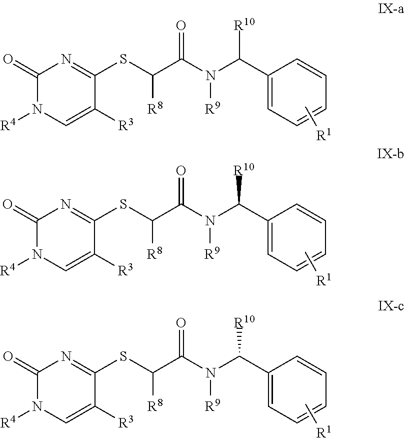

or a pharmaceutically acceptable salt thereof, wherein:
each of R, R¹, R³, R⁴, R⁸, R⁹, R¹⁰, -L¹-, -Cy-, is as defined above and described in embodiments herein, both singly and in combination.

In some embodiments, the present invention provides a compound of Formula X-a, Formula X-b or Formula X-c:

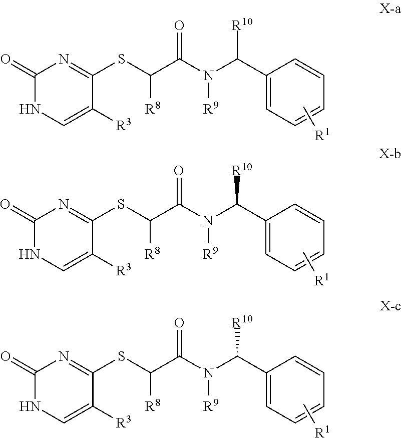

or a pharmaceutically acceptable salt thereof, wherein:
each of R, R¹, R³, R⁸, R⁹, R¹⁰, -L¹-, -Cy-, is as defined above and described in embodiments herein, both singly and in combination.

In some embodiments, the present invention provides a compound of Formula XI-a, Formula XI-b or Formula XI-c:

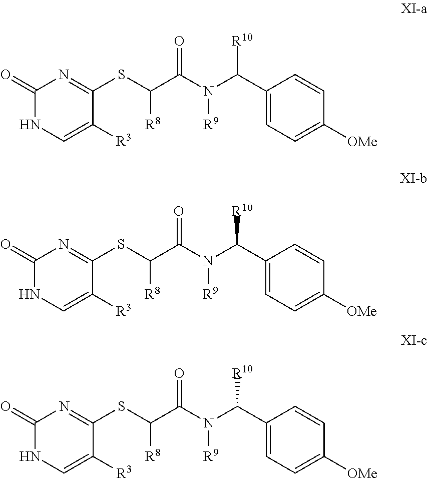

or a pharmaceutically acceptable salt thereof, wherein:
each of R, R³, R⁸, R⁹, R¹⁰, -L¹-, -Cy-, is as defined above and described in embodiments herein, both singly and in combination.

In some embodiments, the present invention provides a compound of Formula XII-a, Formula XII-b or Formula XII-c:

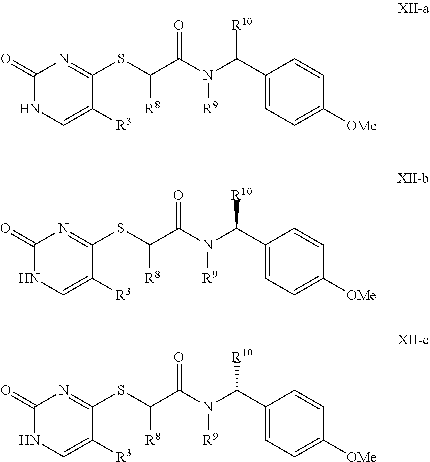

or a pharmaceutically acceptable salt thereof, wherein:
each of R, R³, R⁸, R¹⁰, -L¹-, -Cy-, is as defined above and described in embodiments herein, both singly and in combination.

In some embodiments, the present invention provides a compound of Formula XIII-a, Formula XIII-b or Formula XIII-c:

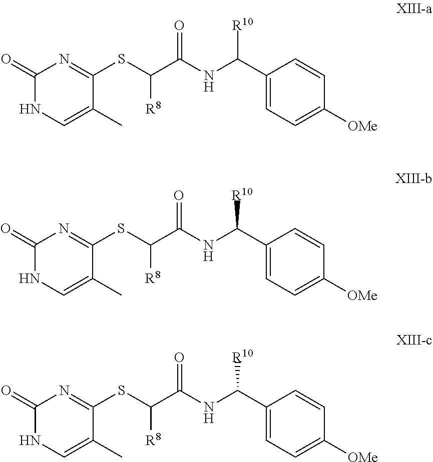

or a pharmaceutically acceptable salt thereof, wherein:
each of R, R⁸, R¹⁰, -L¹-, -Cy-, is as defined above and described in embodiments herein, both singly and in combination.

In some embodiments, the present invention provides a compound of Formula XIV-a, Formula XIV-b or Formula XIV-c:

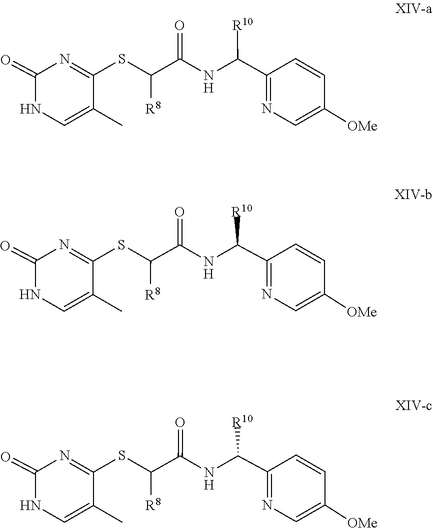

or a pharmaceutically acceptable salt thereof, wherein:
each of R, R¹, R¹⁰, -L¹-, -Cy-, is as defined above and described in embodiments herein, both singly and in combination.

In some embodiments, the present invention provides a compound of Formula XV-a:

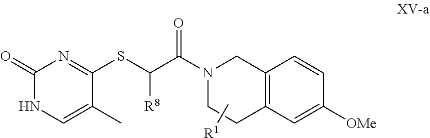

or a pharmaceutically acceptable salt thereof, wherein:
each of R⁸ and R¹ is as defined above and described in embodiments herein, both singly and in combination.

In some embodiments, the present invention provides a compound of Formula XVI-a, Formula XVI-b or Formula XVI-c:

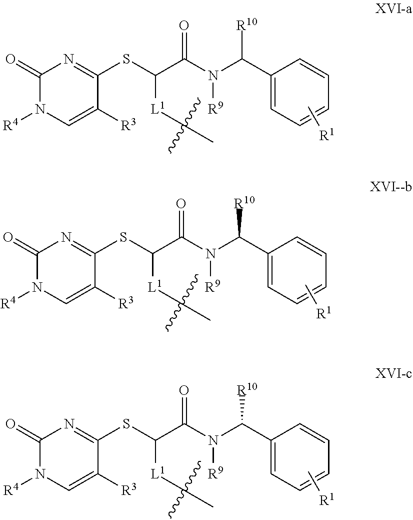

or a pharmaceutically acceptable salt thereof, wherein:
each of R, R¹, R³, R⁴, R⁹, R¹⁰, -L¹-, -Cy-, is as defined above and described in embodiments herein, both singly and in combination.

In some embodiments, the present invention provides a compound of Formula XVII-a, Formula XVII-b or Formula XVII-c:

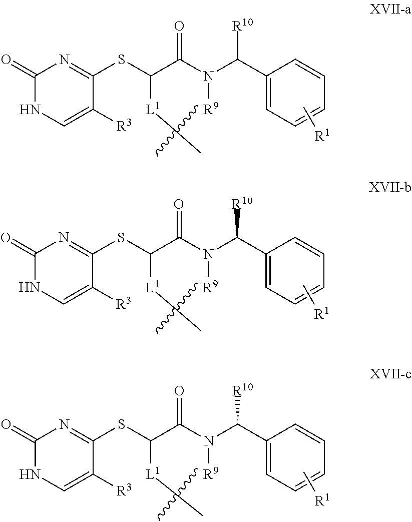

or a pharmaceutically acceptable salt thereof, wherein:
each of R, R¹, R³, R⁹, R¹⁰, -L¹-, -Cy-, is as defined above and described in embodiments herein, both singly and in combination.

In some embodiments, the present invention provides a compound of Formula XVIII-a, Formula XVIII-b or Formula XVIII-c:

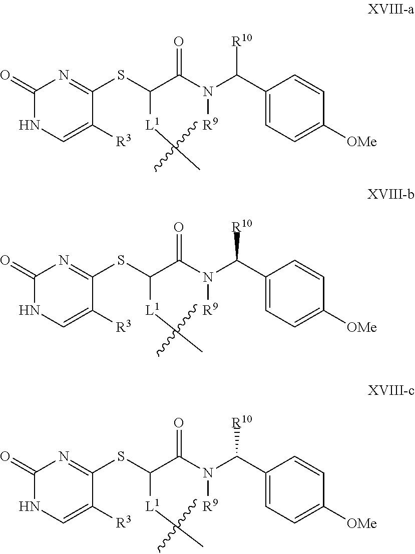

or a pharmaceutically acceptable salt thereof, wherein:
each of R, R³, R⁹, R¹⁰, -L¹-, -Cy-, is as defined above and described in embodiments herein, both singly and in combination.

In some embodiments, the present invention provides a compound of Formula XIX-a, Formula XIX-b or Formula XIX-c:

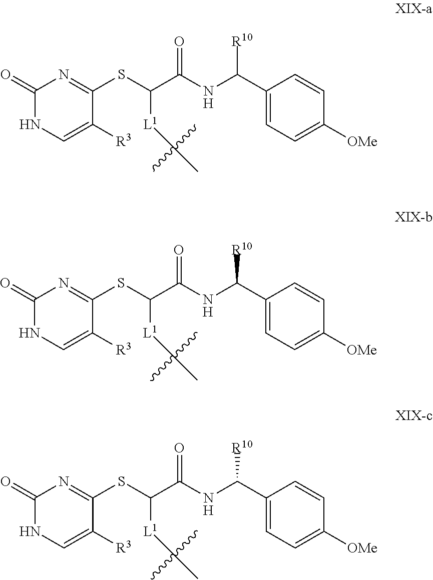

or a pharmaceutically acceptable salt thereof, wherein:
each of R, R³, R¹⁰, -L¹-, -Cy-, is as defined above and described in embodiments herein, both singly and in combination.

In some embodiments, the present invention provides a compound of Formula XX-a, Formula XX-b or Formula XX-c:

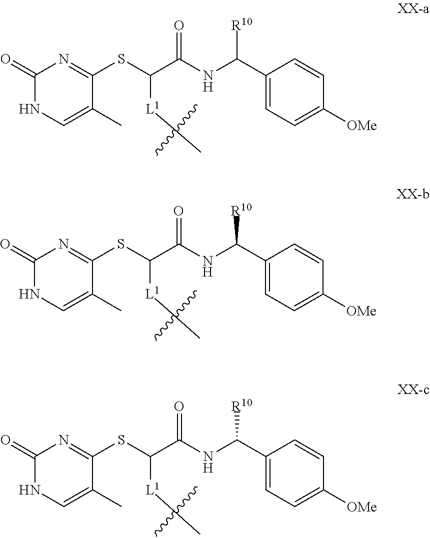

or a pharmaceutically acceptable salt thereof, wherein:
each of R, R¹⁰, -L¹-, -Cy-, is as defined above and described in embodiments herein, both singly and in combination.

In some embodiments, the present invention provides a compound of Formula XXI-a, Formula XXI-b or Formula XXI-c:

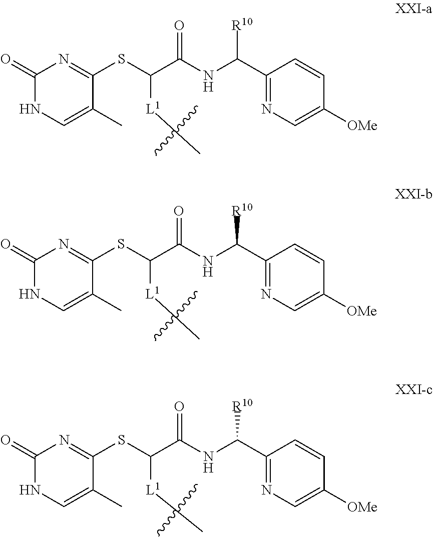

or a pharmaceutically acceptable salt thereof, wherein:
each of R, R¹⁰, -L¹-, -Cy-, is as defined above and described in embodiments herein, both singly and in combination.

In some embodiments, the present invention provides a compound of Formula XXII-a:

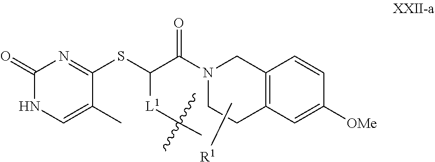

or a pharmaceutically acceptable salt thereof, wherein:
each of -L¹-, -Cy-, and R¹ is as defined above and described in embodiments herein, both singly and in combination.

In some embodiments, the present invention provides a compound of Formula XXIII-a, Formula XXIII-b or Formula XXIII-c:

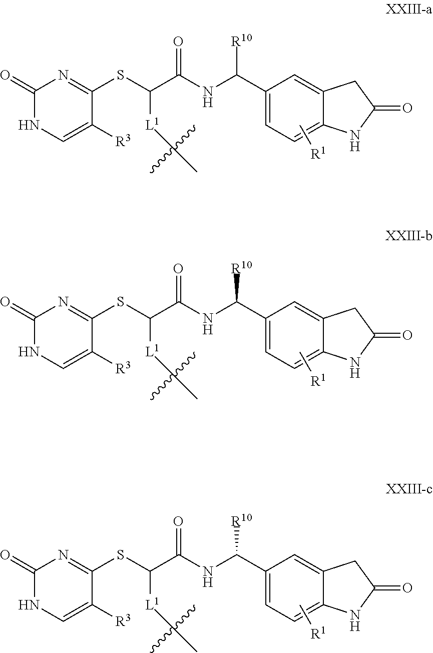

or a pharmaceutically acceptable salt thereof, wherein:
each of R, R¹, R³, R¹⁰, -L¹-, -Cy-, is as defined above and described in embodiments herein, both singly and in combination.

In some embodiments, the present invention provides a compound of Formula XXIV-a:

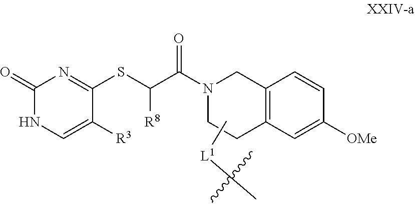

or a pharmaceutically acceptable salt thereof, wherein:
each of -L¹-, -Cy-, R³ and R⁸ is as defined above and described in embodiments herein, both singly and in combination.

In some embodiments, the present invention provides a compound of Formula XXV-a:

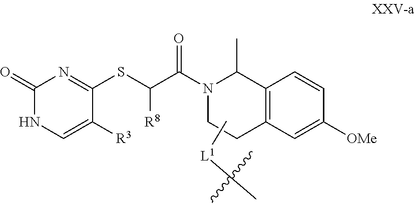

or a pharmaceutically acceptable salt thereof, wherein:
each of -L¹-, -Cy-, R³ and R⁸ is as defined above and described in embodiments herein, both singly and in combination.

In another aspect, the present invention provides a compound of Formula I-b:

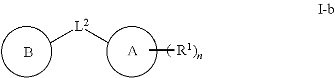

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
Ring B is

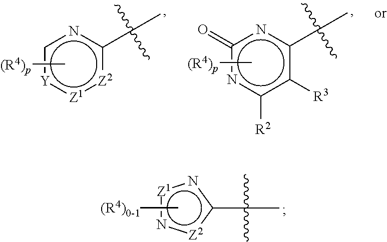

Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R,
-L²- is

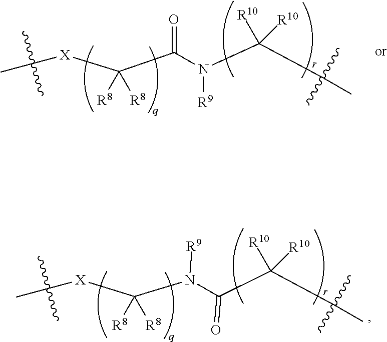

wherein —X— is covalently bound to Ring B; —X— is NR⁶, —O—, —CR⁶R⁷—, or —S—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
R⁹ is —R, halogen, —CN, —NC, —C(O)OR, —OC(O)R, —OR, —N(R)₂, or —SR; or R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur; each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R⁹ and R¹⁰ or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
m is 0, 1, 2, 3, or 4;
n is 0, 1, 2, 3, or 4;
p is 0, 1, 2, or 3;
q is 0, 1, 2, 3, or 4; and
r is 0, 1, 2, 3, or 4.

As defined generally above, Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, Ring A is a 3-8 membered saturated or partially unsaturated monocyclic carbocyclic ring. In some embodiments, Ring A is phenyl. In some embodiments, Ring A is an 8-10 membered bicyclic aromatic carbocyclic ring. In some embodiments, Ring A is a 4-8 membered saturated or partially unsaturated monocyclic heterocyclic ring having 1-3 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is an 8-10 membered bicyclic heteroaromatic ring having 1-5 heteroatoms independently selected from nitrogen, oxygen, and sulfur.

In some embodiments, Ring A is selected from:

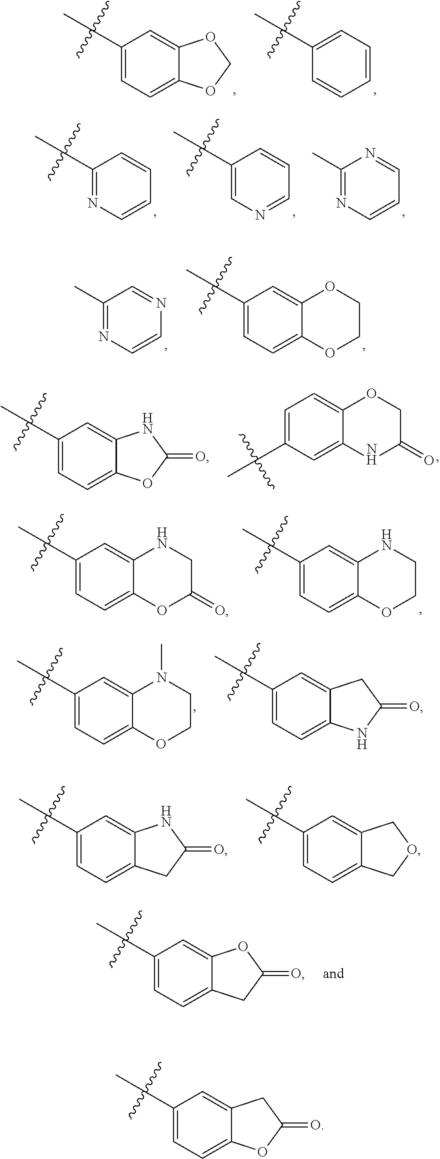

In some embodiments, Ring A is selected from

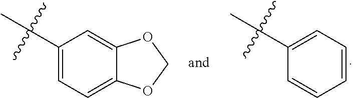 [SMILES: *c1ccc2c(c1)OCO2 ; *c1ccccc1]

In some embodiments, Ring A is selected from those depicted in Table 1, below.

As defined generally above, Ring B is

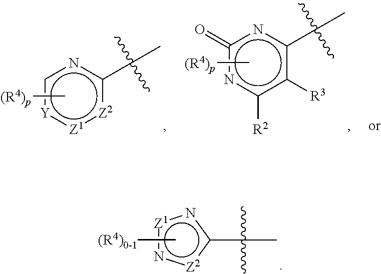

In some embodiments, Ring B is

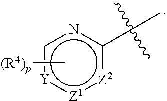

In some embodiments, Ring B is

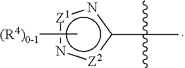

In some embodiments, Ring B is

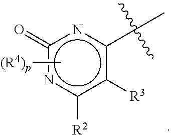

In some embodiments, Ring B is

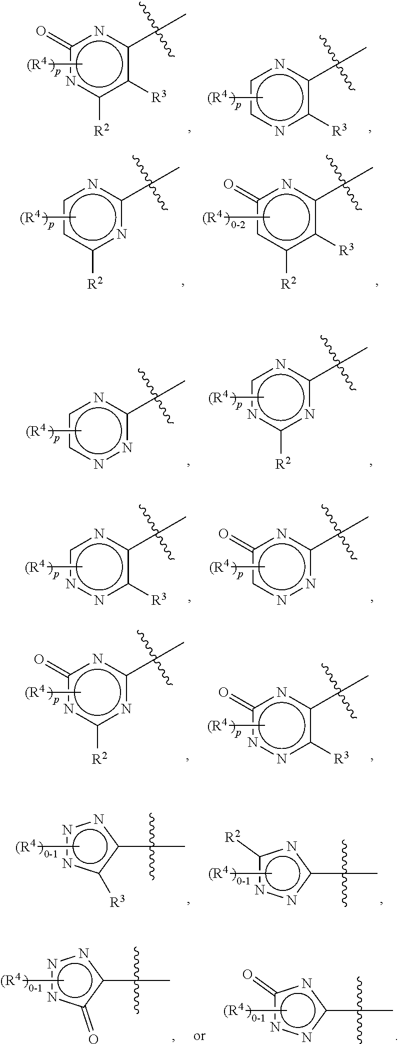

In some embodiments, Ring B is selected from

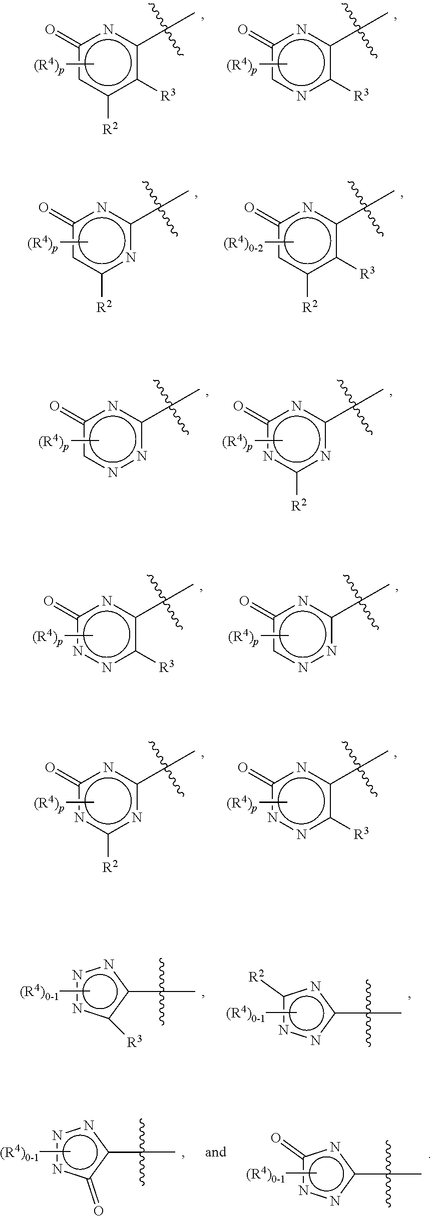

In some embodiments, Ring B is

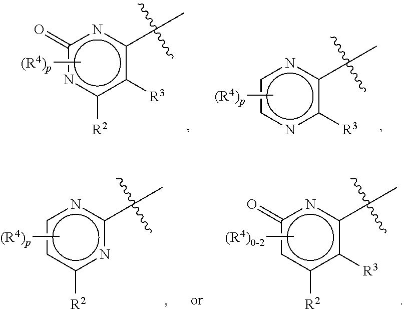

In some embodiments, Ring B is selected from

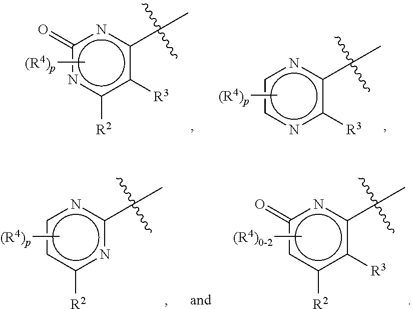

In some embodiments, Ring B is

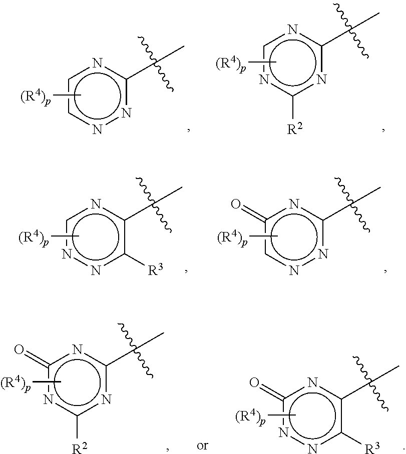

In some embodiments, Ring B is selected from

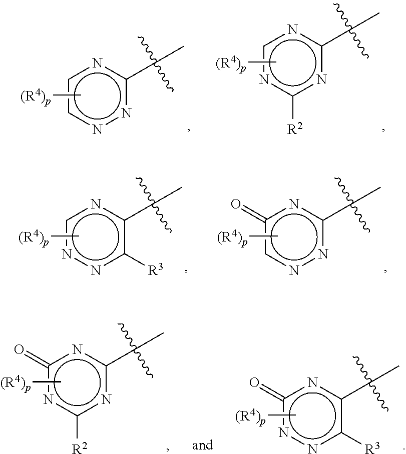

In some embodiments, Ring B is

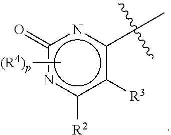

In some embodiments, Ring B is

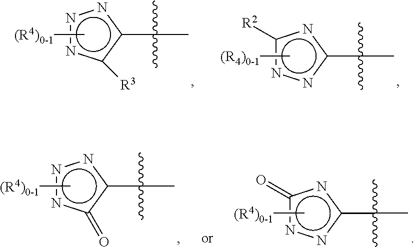

In some embodiments, Ring B is selected from

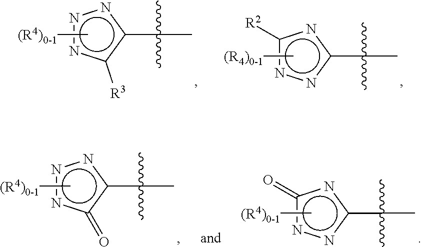

In some embodiments, Ring B is selected from those depicted in Table 1, below.

As defined generally above, Y is N or CH.

In some embodiments, Y is N. In some embodiments, Y is CH.

In some embodiments, Y is CH. In some embodiments, Y is substituted with R⁴.

In some embodiments, Y is selected from those depicted in Table 1, below.

As defined generally above, Z¹ is N, C═O, or CR².

In some embodiments, Z¹ is N. In some embodiments, Z¹ is C═O. In some embodiments, Z¹ is CR².

In some embodiments, Z¹ is selected from those depicted in Table 1, below.

As defined generally above, Z² is N, C═O, or CR³.

In some embodiments, Z² is N. In some embodiments, Z² is C═O. In some embodiments, Z² is CR³.

In some embodiments, Z² is selected from those depicted in Table 1, below.

As defined generally above, each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹ is R. In some embodiments, R¹ is halogen. In some embodiments, R¹ is —CN. In some embodiments, R¹ is —NC. In some embodiments, R¹ is —C(O)OR. In some embodiments, R¹ is —OC(O)R. In some embodiments, R¹ is —C(O)N(R)₂. In some embodiments, R¹ is —N(R)C(O)R. In some embodiments, R¹ is —N(R)C(O)N(R)₂. In some embodiments, R¹ is —OC(O)N(R)₂. In some embodiments, R¹ is —N(R)C(O)OR. In some embodiments, R¹ is —OR. In some embodiments, R¹ is —N(R)₂. In some embodiments, R¹ is —NO₂. In some embodiments, R¹ is —N₃. In some embodiments, R¹ is —SR. In some embodiments, R¹ is —S(O)R. In some embodiments, R is —S(O)₂R. In some embodiments, R¹ is —S(O)₂N(R)₂. In some embodiments, R¹ is —NRS(O)₂R.

In some embodiments, R¹ is hydrogen. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, and sulfur. In some embodiments, R¹ is an optionally substituted 5-6 membered monocyclic heteroaromatic ring having 1-4 heteroatoms independently selected from nitrogen, oxygen, and sulfur. In some embodiments, R¹ is an optionally substituted 8-10 membered bicyclic heteroaromatic ring having 1-5 heteroatoms independently selected from nitrogen, oxygen, and sulfur.

In some embodiments, R¹ is selected from H, F, Cl, OH, Me, Et, i-Pr,

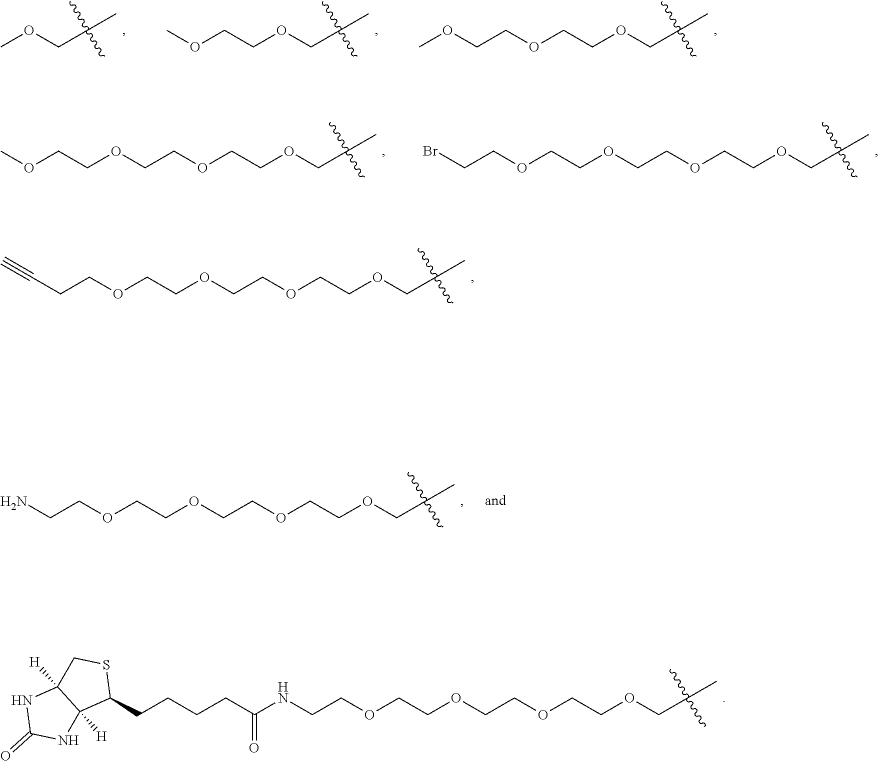

In some embodiments, R¹ is selected from those depicted in Table 1, below.

As defined generally above, each R² and R³ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² is R. In some embodiments, R² is halogen. In some embodiments, R² is —CN. In some embodiments, R² is —NC. In some embodiments, R² is —C(O)OR. In some embodiments, R² is —OC(O)R. In some embodiments, R² is —C(O)N(R)₂. In some embodiments, R² is —N(R)C(O)R. In some embodiments, R² is —N(R)C(O)N(R)₂. In some embodiments, R² is —OC(O)N(R)₂. In some embodiments, R² is —N(R)C(O)OR. In some embodiments, R² is —OR. In some embodiments, R² is —N(R)₂. In some embodiments, R² is —NO₂. In some embodiments, R² is —N₃. In some embodiments, R² is —SR. In some embodiments, R² is —S(O)R. In some embodiments, R² is —S(O)₂R. In some embodiments, R² is —S(O)₂N(R)₂. In some embodiments, R² is —NRS(O)₂R.

In some embodiments, R² is hydrogen. In some embodiments, R² is an optionally substituted C₁₋₆ aliphatic group. In some embodiments, R² is an optionally substituted 4-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, and sulfur. In some embodiments, R² is an optionally substituted 5-6 membered monocyclic heteroaromatic ring having 1-4 heteroatoms independently selected from nitrogen, oxygen, and sulfur. In some embodiments, R² is an optionally substituted 8-10 membered bicyclic heteroaromatic ring having 1-5 heteroatoms independently selected from nitrogen, oxygen, and sulfur.

In some embodiments, R² is selected from H, F, Cl, OH, Me, Et, i-Pr,

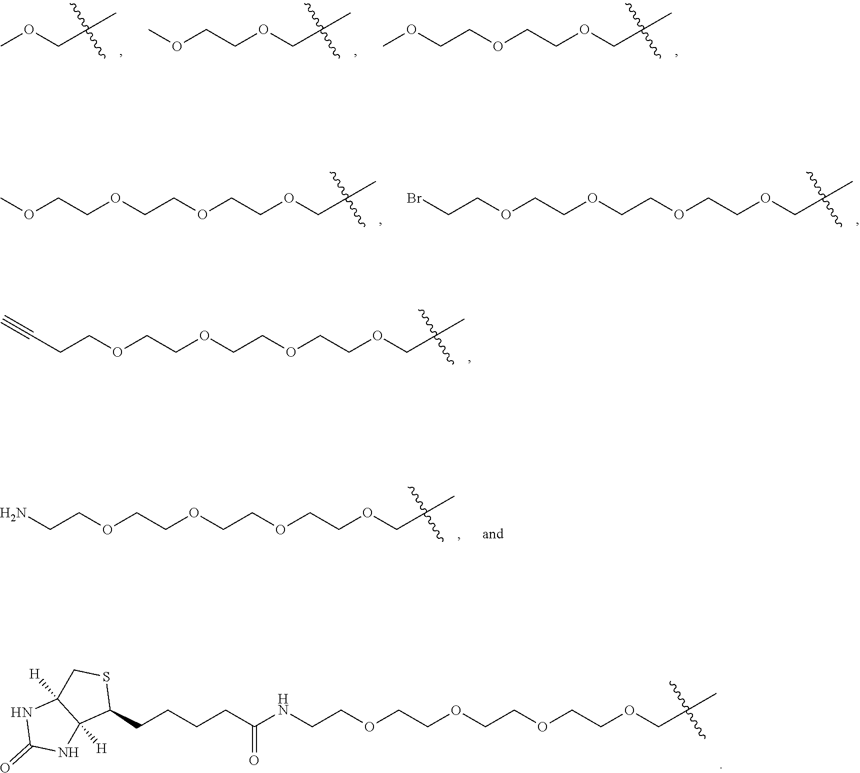

In some embodiments, R³ is R. In some embodiments, R³ is halogen. In some embodiments, R³ is —CN. In some embodiments, R³ is —NC. In some embodiments, R³ is —C(O)OR. In some embodiments, R³ is —OC(O)R. In some embodiments, R³ is —C(O)N(R)₂. In some embodiments, R³ is —N(R)C(O)R. In some embodiments, R³ is —N(R)C(O)N(R)₂. In some embodiments, R³ is —OC(O)N(R)₂. In some embodiments, R³ is —N(R)C(O)OR. In some embodiments, R³ is —OR. In some embodiments, R³ is —N(R)₂. In some embodiments, R³ is —NO₂. In some embodiments, R³ is —N₃. In some embodiments, R³ is —SR. In some embodiments, R³ is —S(O)R. In some embodiments, R³ is —S(O)₂R. In some embodiments, R³ is —S(O)₂N(R)₂. In some embodiments, R³ is —NRS(O)₂R.

In some embodiments, R³ is hydrogen. In some embodiments, R³ is an optionally substituted C₁₋₆ aliphatic group. In some embodiments, R³ is an optionally substituted 4-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, and sulfur. In some embodiments, R³ is an optionally substituted 5-6 membered monocyclic heteroaromatic ring having 1-4 heteroatoms independently selected from nitrogen, oxygen, and sulfur. In some embodiments, R³ is an optionally substituted 8-10 membered bicyclic heteroaromatic ring having 1-5 heteroatoms independently selected from nitrogen, oxygen, and sulfur.

In some embodiments, R³ is selected from H, F, Cl, OH, Me, Et, i-Pr,

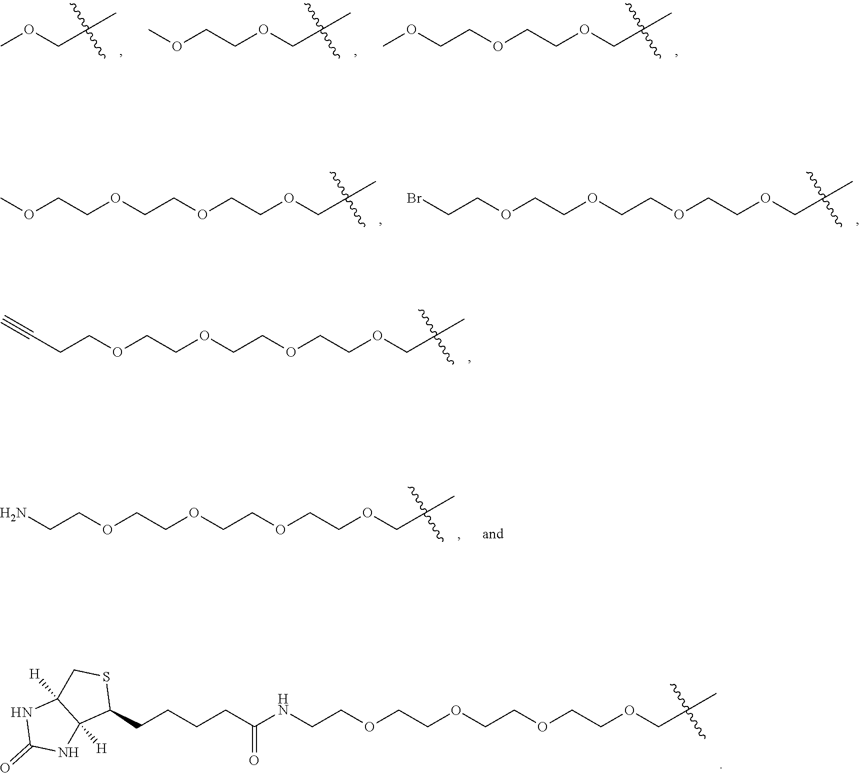

In some embodiments, R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² and R³ taken together with the carbons to which they are attached form a 4-8 membered saturated or partially unsaturated monocyclic carbocyclic ring. In some embodiments, R² and R³ taken together with the carbons to which they are attached form phenyl. In some embodiments, R² and R³ taken together with the carbons to which they are attached form an 8-10 membered bicyclic aromatic carbocyclic ring. In some embodiments, R² and R³ taken together with the carbons to which they are attached form a 4-8 membered saturated or partially unsaturated monocyclic heterocyclic ring having 1-2 heteroatoms independently selected from nitrogen, oxygen, and sulfur. In some embodiments, R² and R³ taken together with the carbons to which they are attached form a 5-6 membered monocyclic heteroaromatic ring having 1-4 heteroatoms independently selected from nitrogen, oxygen, and sulfur. In some embodiments, R² and R³ taken together with the carbons to which they are attached form an 8-10 membered bicyclic heteroaromatic ring having 1-5 heteroatoms independently selected from nitrogen, oxygen, and sulfur.

In some embodiments, R² and R³ taken together with the carbons to which they are attached form a cyclohexane ring. In some embodiments, R² and R³ taken together with the carbons to which they are attached form a cyclopentane ring.

In some embodiments, R² and R³ are selected from those depicted in Table 1, below.

As defined generally above, each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁴ is R. In some embodiments, R⁴ is halogen. In some embodiments, R⁴ is ═O. In some embodiments, R⁴ is —CN. In some embodiments, R⁴ is —NC. In some embodiments, R⁴ is —C(O)OR. In some embodiments, R⁴ is —OC(O)R. In some embodiments, R⁴ is —C(O)N(R)₂. In some embodiments, R⁴ is —N(R)C(O)R. In some embodiments, R⁴ is —N(R)C(O)N(R)₂. In some embodiments, R⁴ is —OC(O)N(R)₂. In some embodiments, R⁴ is —N(R)C(O)OR. In some embodiments, R⁴ is —OR. In some embodiments, R⁴ is —N(R)₂. In some embodiments, R⁴ is —NO₂. In some embodiments, R⁴ is —N₃. In some embodiments, R⁴ is —SR. In some embodiments, R⁴ is —S(O)R. In some embodiments, R⁴ is —S(O)₂R. In some embodiments, R⁴ is —S(O)₂N(R)₂. In some embodiments, R⁴ is —NRS(O)₂R.

In some embodiments, R⁴ is hydrogen. In some embodiments, R⁴ is an optionally substituted C₁₋₆ aliphatic group. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, and sulfur. In some embodiments, R⁴ is an optionally substituted 5-6 membered monocyclic heteroaromatic ring having 1-4 heteroatoms independently selected from nitrogen, oxygen, and sulfur. In some embodiments, R⁴ is an optionally substituted 8-10 membered bicyclic heteroaromatic ring having 1-5 heteroatoms independently selected from nitrogen, oxygen, and sulfur.

In some embodiments, R⁴ is selected from H, ═O, Me, Et, iPr,

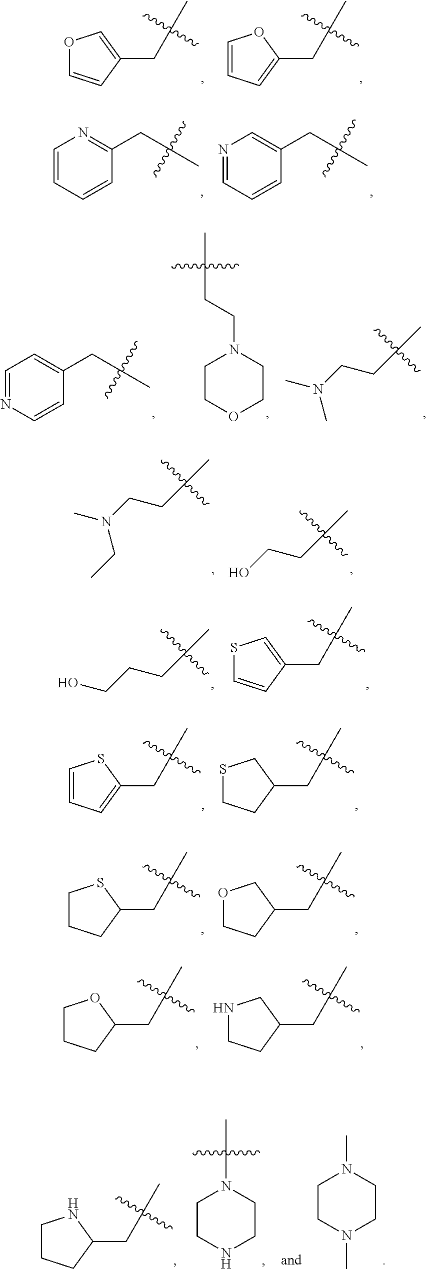

In some embodiments, R⁴ is selected from those depicted in Table 1, below.

As defined generally above, each R⁵ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁵ is R. In some embodiments, R⁵ is halogen. In some embodiments, R⁵ is —CN. In some embodiments, R⁵ is —NC. In some embodiments, R⁵ is —C(O)OR. In some embodiments, R⁵ is —OC(O)R. In some embodiments, R⁵ is —C(O)N(R)₂. In some embodiments, R⁵ is —N(R)C(O)R. In some embodiments, R⁵ is —N(R)C(O)N(R)₂. In some embodiments, R⁵ is —OC(O)N(R)₂. In some embodiments, R⁵ is —N(R)C(O)OR. In some embodiments, R⁵ is —OR. In some embodiments, R⁵ is —N(R)₂. In some embodiments, R⁵ is —NO₂. In some embodiments, R⁵ is —N₃. In some embodiments, R⁵ is —SR. In some embodiments, R⁵ is —S(O)R. In some embodiments, R⁵ is —S(O)₂R. In some embodiments, R⁵ is —S(O)₂N(R)₂. In some embodiments, R⁵ is —NRS(O)₂R.

In some embodiments, R⁵ is selected from those depicted in Table 1, below.

As defined generally above, -L²- is

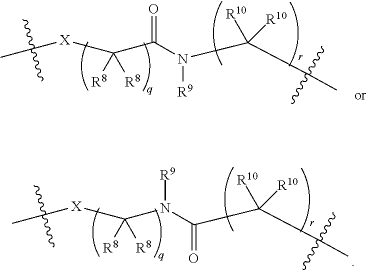

In some embodiments, -L²- is

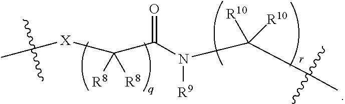

In some embodiments, -L²- is

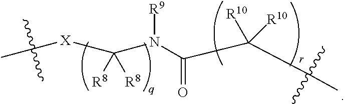

As defined generally above, X is NR⁶, O, CR⁶R⁷ or S.

As defined generally above, each R⁶ and R⁷ is independently —R, halogen, —CN, —NC, —C(O)OR, —OC(O)R, —OR, —N(R)₂, or —SR.

In some embodiments, R⁶ is R. In some embodiments, R⁶ is halogen. In some embodiments, R⁶ is —CN. In some embodiments, R⁶ is —NC. In some embodiments, R⁶ is —C(O)OR. In some embodiments, R⁶ is —OC(O)R. In some embodiments, R⁶ is —OR. In some embodiments, R⁶ is —N(R)₂. In some embodiments, R⁶ is —SR.

In some embodiments, R⁶ is hydrogen. In some embodiments, R⁶ is an optionally substituted C₁₋₆ aliphatic group. In some embodiments, R⁶ is an optionally substituted 3-8 membered saturated or partially unsaturated monocyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 4-8 membered saturated or partially unsaturated monocyclic heterocyclic ring having 1-2 heteroatoms independently selected from nitrogen, oxygen, and sulfur. In some embodiments, R⁶ is an optionally substituted 5-6 membered monocyclic heteroaromatic ring having 1-4 heteroatoms independently selected from nitrogen, oxygen, and sulfur. In some embodiments, R⁶ is an optionally substituted 8-10 membered bicyclic heteroaromatic ring having 1-5 heteroatoms independently selected from nitrogen, oxygen, and sulfur.

In some embodiments, R⁶ is selected from H, Me, Et, iPr and OH.

In some embodiments, R⁶ is selected from those depicted in Table 1, below.

In some embodiments, R⁷ is R. In some embodiments, R⁷ is halogen. In some embodiments, R⁷ is —CN. In some embodiments, R⁷ is —NC. In some embodiments, R⁷ is —C(O)OR. In some embodiments, R⁷ is —OC(O)R. In some embodiments, R⁷ is —OR. In some embodiments, R⁷ is —N(R)₂. In some embodiments, R⁷ is —SR.

In some embodiments, R⁷ is hydrogen. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, and sulfur. In some embodiments, R⁷ is an optionally substituted 5-6 membered monocyclic heteroaromatic ring having 1-4 heteroatoms independently selected from nitrogen, oxygen, and sulfur. In some embodiments, R⁷ is an optionally substituted 8-10 membered bicyclic heteroaromatic ring having 1-5 heteroatoms independently selected from nitrogen, oxygen, and sulfur.

In some embodiments, R⁷ is selected from H, Me, Et, iPr and OH.

In some embodiments, R⁷ is selected from those depicted in Table 1, below.

In some embodiments, X is selected from NH, O, S, NMe, CHMe, CHOH, C(Me)₂ and C(Me)OH.

In some embodiments, X is selected from those depicted in Table 1, below.

As defined generally above, each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁸ is R. In some embodiments, R⁸ is halogen. In some embodiments, R⁸ is —CN. In some embodiments, R⁸ is —NC. In some embodiments, R⁸ is —C(O)OR. In some embodiments, R⁸ is —OC(O)R. In some embodiments, R⁸ is —C(O)N(R)₂. In some embodiments, R⁸ is —N(R)C(O)R. In some embodiments, R⁸ is —N(R)C(O)N(R)₂. In some embodiments, R⁸ is —OC(O)N(R)₂. In some embodiments, R⁸ is —N(R)C(O)OR. In some embodiments, R⁸ is —OR. In some embodiments, R⁸ is —N(R)₂. In some embodiments, R⁸ is —NO₂. In some embodiments, R⁸ is —N₃. In some embodiments, R⁸ is —SR. In some embodiments, R⁸ is —S(O)R. In some embodiments, R⁸ is —S(O)₂R. In some embodiments, R⁸ is —S(O)₂N(R)₂. In some embodiments, R⁸ is —NRS(O)₂R.

In some embodiments, R⁸ is hydrogen. In some embodiments, R⁸ is an optionally substituted C₁₋₆ aliphatic group. In some embodiments, R⁸ is an optionally substituted 3-8 membered saturated or partially unsaturated monocyclic carbocyclic ring. In some embodiments, R⁸ is an optionally substituted phenyl. In some embodiments, R⁸ is an optionally substituted 8-10 membered bicyclic aromatic carbocyclic ring. In some embodiments, R⁸ is an optionally substituted 4-8 membered saturated or partially unsaturated monocyclic heterocyclic ring having 1-2 heteroatoms independently selected from nitrogen, oxygen, and sulfur. In some embodiments, R⁸ is an optionally substituted 5-6 membered monocyclic heteroaromatic ring having 1-4 heteroatoms independently selected from nitrogen, oxygen, and sulfur. In some embodiments, R⁸ is an optionally substituted 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring. In some embodiments, one instance of —C(R⁸)₂— is optionally replaced by phenyl. In some embodiments, one instance of —C(R⁸)₂— is optionally replaced by an 8-10 membered bicyclic aromatic carbocyclic ring. In some embodiments, one instance of —C(R⁸)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R⁸)₂— is optionally replaced by a 5-6 membered monocyclic heteroaromatic ring having 1-4 heteroatoms independently selected from nitrogen, oxygen, and sulfur. In some embodiments, one instance of —C(R⁸)₂— is optionally replaced by an 8-10 membered bicyclic heteroaromatic ring having 1-5 heteroatoms independently selected from nitrogen, oxygen, and sulfur.

In some embodiments, one instance of —C(R⁸)₂— is optionally replaced by

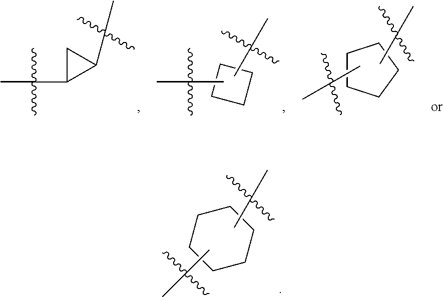

In some embodiments, R⁸ is selected from H, F, Cl, OH, Me, Et, i-Pr,

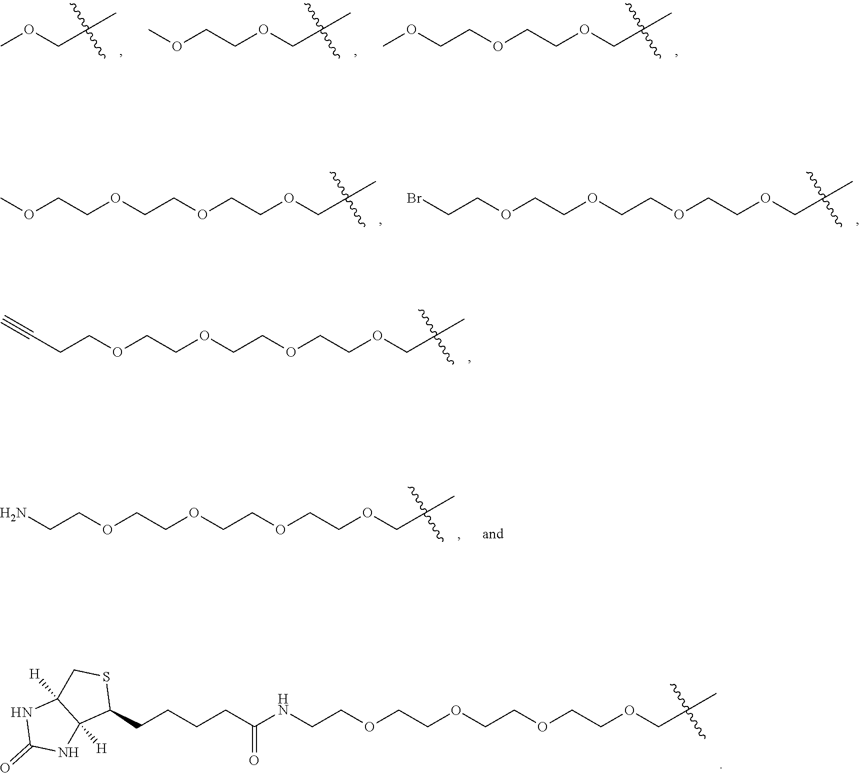

In some embodiments, R⁸ is selected from those depicted in Table 1, below.

As defined generally above, R⁹ is —R, halogen, —CN, —NC, —C(O)OR, —OC(O)R, —OR, —N(R)₂, or —SR.

In some embodiments, R⁹ is R. In some embodiments, R⁹ is halogen. In some embodiments, R⁹ is —CN. In some embodiments, R⁹ is —NC. In some embodiments, R⁹ is —C(O)OR. In some embodiments, R⁹ is —OC(O)R. In some embodiments, R⁹ is —OR. In some embodiments, R⁹ is —N(R)₂. In some embodiments, R⁹ is —SR.

In some embodiments, R⁹ is hydrogen. In some embodiments, R⁹ is an optionally substituted C₁₋₆ aliphatic group. In some embodiments, R⁹ is an optionally substituted 3-8 membered saturated or partially unsaturated monocyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 4-8 membered saturated or partially unsaturated monocyclic heterocyclic ring having 1-2 heteroatoms independently selected from nitrogen, oxygen, and sulfur. In some embodiments, R⁹ is an optionally substituted 5-6 membered monocyclic heteroaromatic ring having 1-4 heteroatoms independently selected from nitrogen, oxygen, and sulfur. In some embodiments, R⁹ is an optionally substituted 8-10 membered bicyclic heteroaromatic ring having 1-5 heteroatoms independently selected from nitrogen, oxygen, and sulfur.

In some embodiments, R⁹ is selected from H, Me, Et, iPr and OH.

In some embodiments, R⁹ is selected from those depicted in Table 1, below.

In some embodiments, R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur.

As defined generally above, R¹⁰ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹⁰ is R. In some embodiments, R¹⁰ is halogen. In some embodiments, R¹⁰ is —CN. In some embodiments, R¹⁰ is —NC. In some embodiments, R¹⁰ is —C(O)OR. In some embodiments, R¹⁰ is —OC(O)R. In some embodiments, R¹⁰ is —C(O)N(R)₂. In some embodiments, R¹⁰ is —N(R)C(O)R. In some embodiments, R¹⁰ is —N(R)C(O)N(R)₂. In some embodiments, R¹⁰ is —OC(O)N(R)₂. In some embodiments, R¹⁰ is —N(R)C(O)OR. In some embodiments, R¹⁰ is —OR. In some embodiments, R¹⁰ is —N(R)₂. In some embodiments, R¹⁰ is —NO₂. In some embodiments, R¹⁰ is —N₃. In some embodiments, R¹⁰ is —SR. In some embodiments, R¹⁰ is —S(O)R. In some embodiments, R¹⁰ is —S(O)₂R. In some embodiments, R¹⁰ is —S(O)₂N(R)₂. In some embodiments, R¹⁰ is —NRS(O)₂R.

In some embodiments, R¹⁰ is hydrogen. In some embodiments, R¹⁰ is an optionally substituted C₁₋₆ aliphatic group. In some embodiments, R¹⁰ is an optionally substituted 3-8 membered saturated or partially unsaturated monocyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 4-8 membered saturated or partially unsaturated monocyclic heterocyclic ring having 1-2 heteroatoms independently selected from nitrogen, oxygen, and sulfur. In some embodiments, R¹⁰ is an optionally substituted 5-6 membered monocyclic heteroaromatic ring having 1-4 heteroatoms independently selected from nitrogen, oxygen, and sulfur. In some embodiments, R¹⁰ is an optionally substituted 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring. In some embodiments, one instance of —C(R¹⁰)₂— is optionally replaced by phenyl. In some embodiments, one instance of —C(R¹⁰)₂— is optionally replaced by an 8-10 membered bicyclic aromatic carbocyclic ring. In some embodiments, one instance of —C(R¹⁰)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R¹⁰)₂— is optionally replaced by a 5-6 membered monocyclic heteroaromatic ring having 1-4 heteroatoms independently selected from nitrogen, oxygen, and sulfur. In some embodiments, one instance of —C(R¹⁰)₂— is optionally replaced by an 8-10 membered bicyclic heteroaromatic ring having 1-5 heteroatoms independently selected from nitrogen, oxygen, and sulfur.

In some embodiments, one instance of —C(R¹⁰)₂— is optionally replaced by

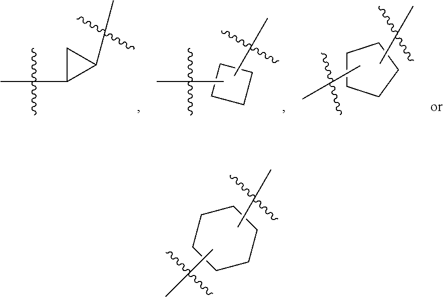

In some embodiments, R¹⁰ is selected from H, Me, Et, iPr and CH₃OH.

In some embodiments, R¹⁰ is selected from those depicted in Table 1, below.

In some embodiments, R⁸ and R¹⁰ taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur.

In some embodiments, R⁹ and R¹⁰ taken together with the atoms to which they are attached, form a ring substituted with m instances of R⁵; wherein the ring is a 4-8 membered saturated or partially unsaturated monocyclic heterocyclic ring having 1-2 heteroatoms independently selected from nitrogen, oxygen, and sulfur.

In some embodiments, -L²- is selected from

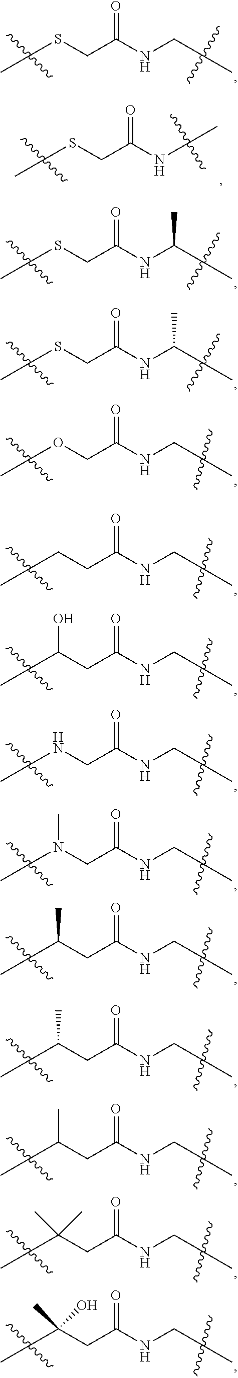

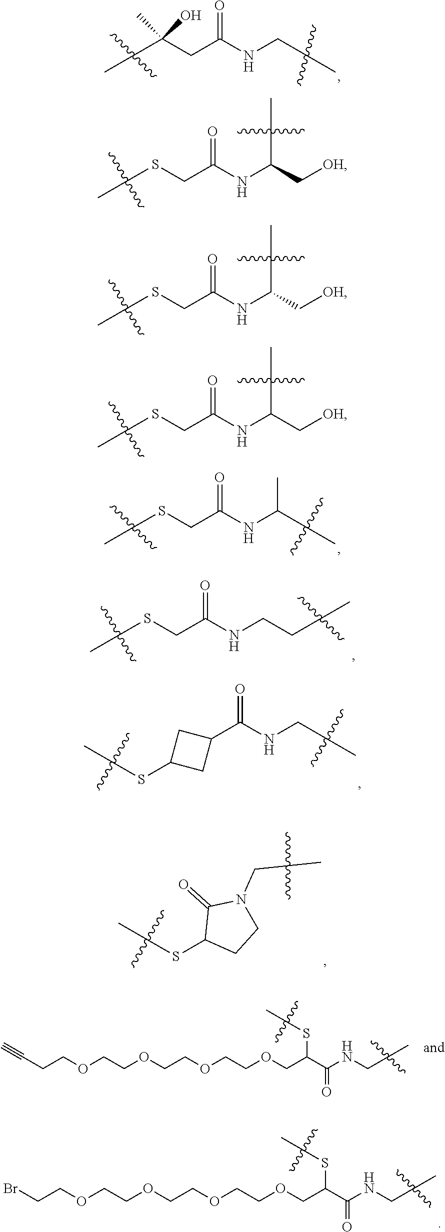

In some embodiments, -L²- is selected from those depicted in Table 1, below.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0, 1, 2, or 3. In some embodiments, m is 0, 1, or 2. In some embodiments, m is 1, 2, or 3.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0, 1, 2, or 3. In some embodiments, n is 0, 1, or 2. In some embodiments, n is 1, 2, or 3.

As defined generally above, p is 0, 1, 2, or 3. In some embodiments, p is 0. In some embodiments, p is 1. In some embodiments, p is 2. In some embodiments, p is 3. In some embodiments, p is 0, 1, 2, or 3. In some embodiments, p is 0, 1, or 2. In some embodiments, p is 1, 2, or 3.

As defined generally above, q is 0, 1, 2, 3, or 4. In some embodiments, q is 0. In some embodiments, q is 1. In some embodiments, q is 2. In some embodiments, q is 3. In some embodiments, q is 4. In some embodiments, q is 0, 1, 2, or 3. In some embodiments, q is 0, 1, or 2. In some embodiments, q is 1, 2, or 3.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0, 1, 2, or 3. In some embodiments, r is 0, 1, or 2. In some embodiments, r is 1, 2, or 3.

Exemplary DFL compounds of the invention are set forth in Table 1 and Table 1A, below.

Exemplary DFL compounds of the invention are set forth in Table 1, Table 1A, and Table 1B below.

Exemplary DFL compounds of the invention are set forth in Table 1, Table 1A, Table 1B and Table 1C below.

In some embodiments, the present invention provides a compound of Formula I-d:

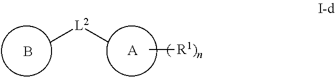

or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur;
Ring B is

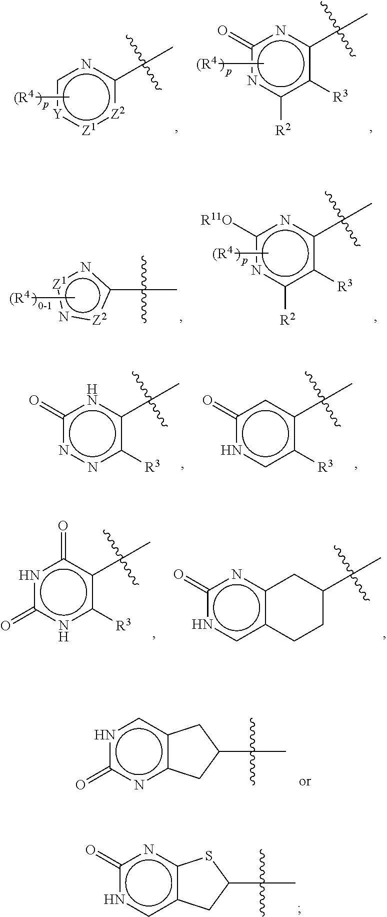

Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is

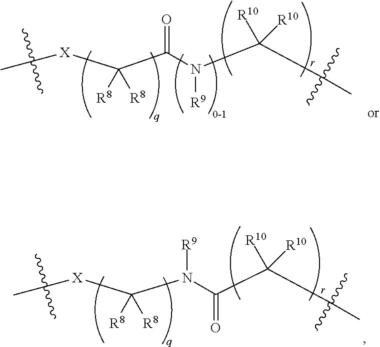

wherein —X— is covalently bound to Ring B; —X— is a bond, —NR⁶, —O—, —CR⁶R⁷—, —C(O)—, —S—, or —S(O)₂—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or two R⁸, taken together with the carbon atom to which they are attached, form a 3-6 membered carbocyclic ring;
R⁹ is —R, halogen, —CN, —NC, —C(O)OR, —OC(O)R, —OR, —N(R)₂, or —SR; or R⁸ and R⁹, taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur;
each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R; or two R¹⁰ taken together with the carbon atom to which they are attached, form a 3-6 membered carbocyclic ring; or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
R¹¹ is H, or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—;
m is 0, 1, 2, 3, or 4;
n is 0, 1, 2, 3, 4, or 5;
p is 0, 1, 2, or 3;
q is 0, 1, 2, 3, or 4; and
r is 0, 1, 2, 3, or 4.

As defined generally above, Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur.

In some embodiments, Ring A is a 3-8 membered saturated or partially unsaturated monocyclic carbocyclic ring. In some embodiments, Ring A is phenyl. In some embodiments, Ring A is an 8-10 membered bicyclic aromatic carbocyclic ring. In some embodiments, Ring A is a 4-8 membered saturated or partially unsaturated monocyclic heterocyclic ring having 1-3 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is an 8-10 membered bicyclic heteroaromatic ring having 1-5 heteroatoms independently selected from nitrogen, oxygen, and sulfur. In some embodiments, Ring A is an 8-12 membered bicyclic heterocyclic ring having 1-5 heteroatoms independently selected from nitrogen, oxygen, and sulfur. In some embodiments, Ring A is an 8-12 membered tricyclic heterocyclic ring having 1-5 heteroatoms independently selected from nitrogen, oxygen, and sulfur.

In some embodiments, Ring A is selected from:

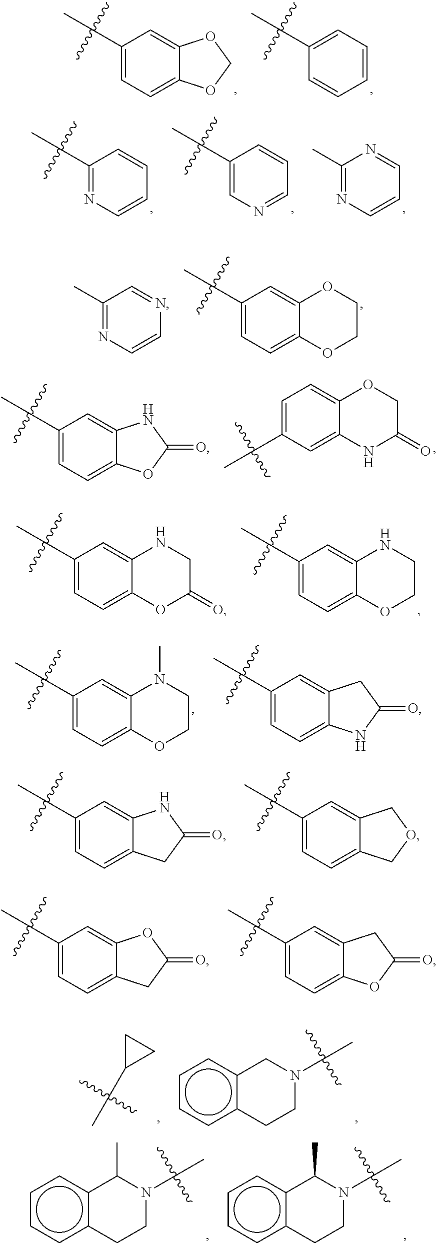

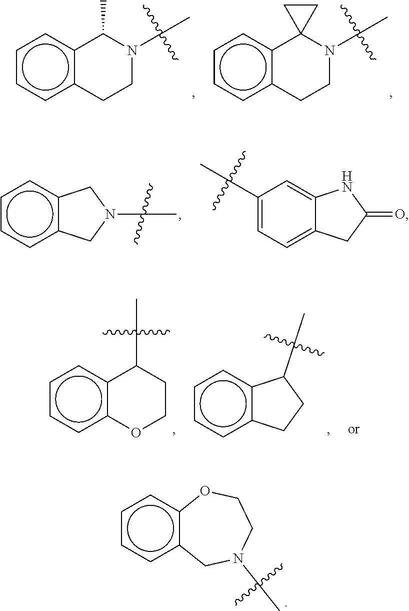

In some embodiments, Ring A is selected from

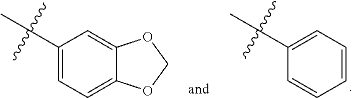 [SMILES: *c1ccc2c(c1)OCO2 ; *c1ccccc1]

In some embodiments, Ring A is selected from those depicted in Table 1, below.

In some embodiments, Ring A is selected from those depicted in Table 1A, below.

In some embodiments, Ring A is selected from those depicted in Table 1B, below.

In some embodiments, Ring A is selected from those depicted in Table 1C, below.

As defined generally above, Ring B is

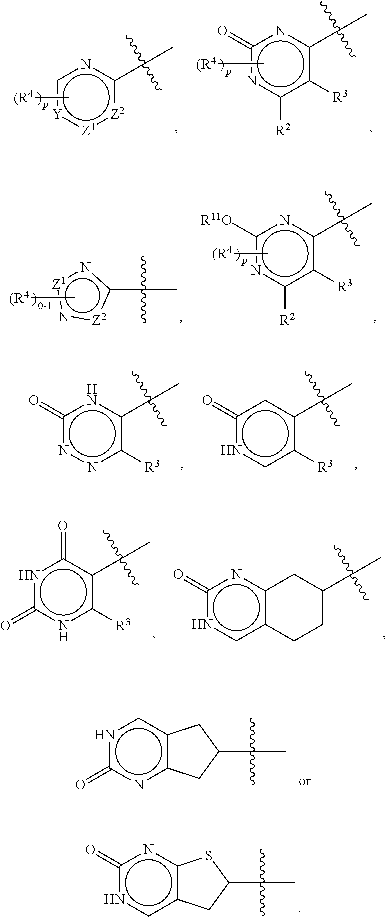

In some embodiments, Ring B is

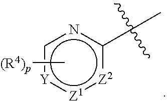

In some embodiments, Ring B is

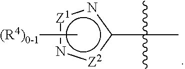

In some embodiments, Ring B is

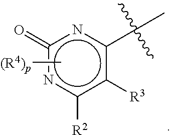

In some embodiments, Ring B is

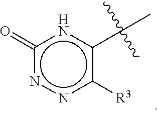

In some embodiments, Ring B is

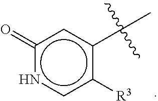

In some embodiments, Ring B is

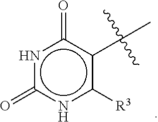

In some embodiments, Ring B is

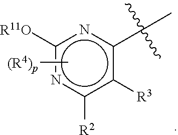

In some embodiments, Ring B is

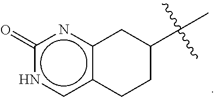

In some embodiments, Ring B is

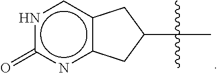

In some embodiments, Ring B is

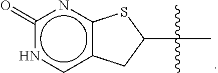

In some embodiments, Ring B is

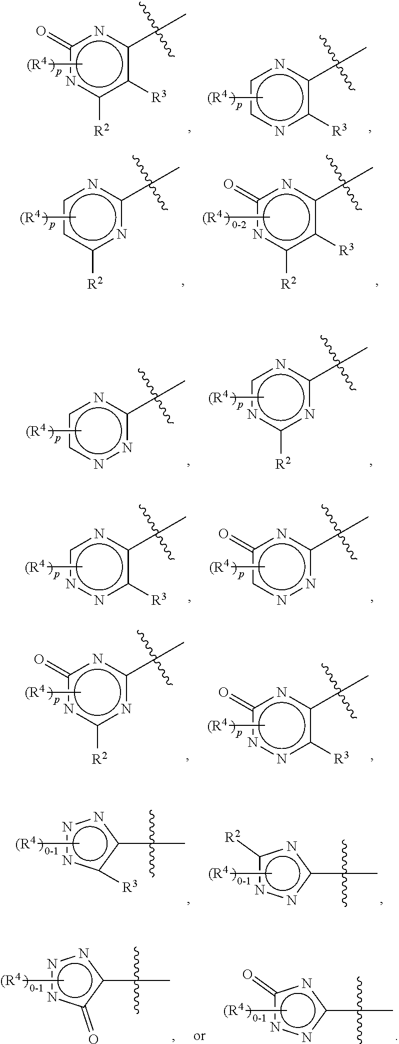

In some embodiments, Ring B is selected from

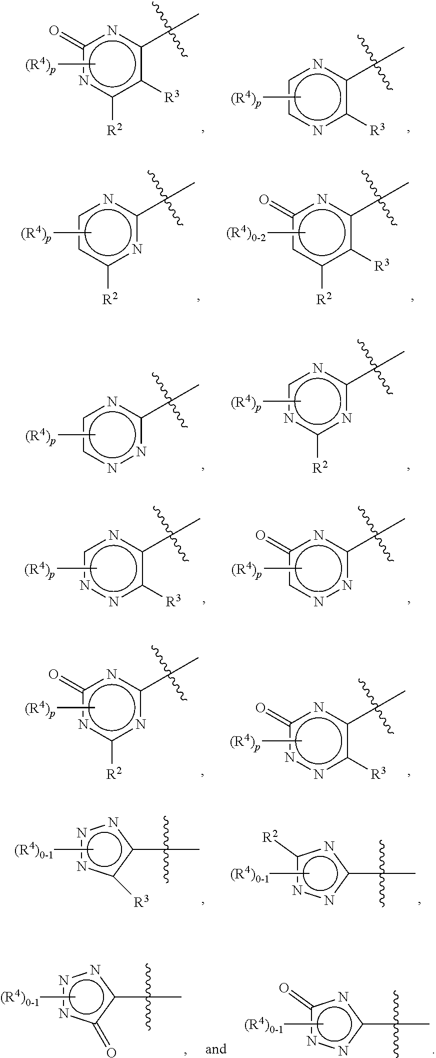

In some embodiments, Ring B is

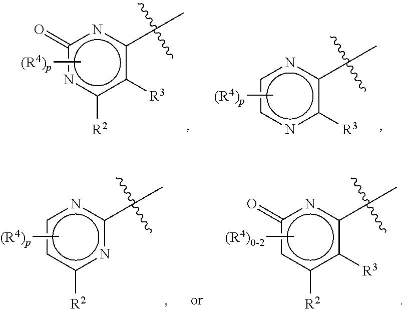

In some embodiments, Ring B is selected from

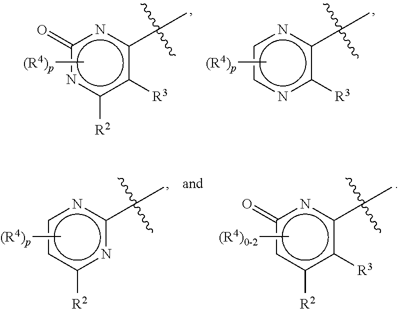

In some embodiments, Ring B is

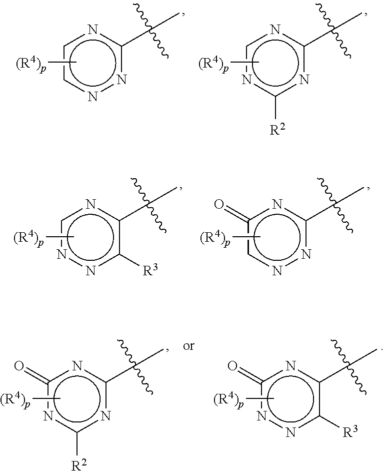

In some embodiments, Ring B is selected from

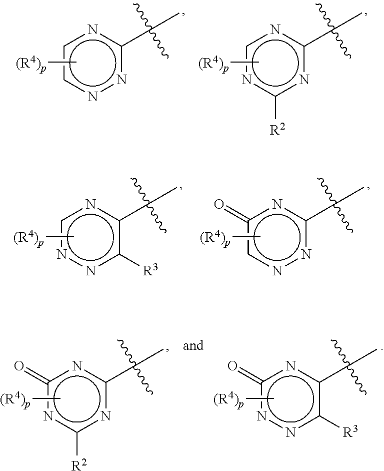

In some embodiments, Ring B is

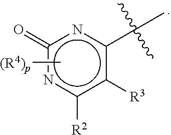

In some embodiments, Ring B is

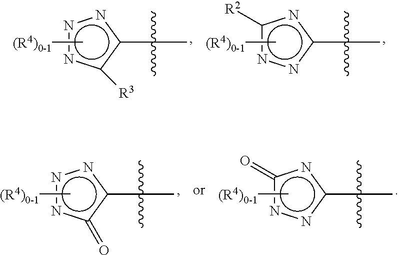

In some embodiments, Ring B is selected from

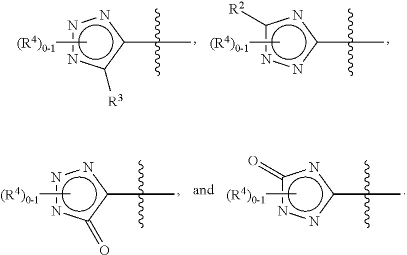

In some embodiments, Ring B is selected from those depicted in Table 1, below.

In some embodiments, Ring B is selected from those depicted in Table 1A, below.

In some embodiments, Ring B is selected from those depicted in Table 1B, below.

In some embodiments, Ring B is selected from those depicted in Table 1C, below.

As defined generally above, Y is N or CH.

In some embodiments, Y is N. In some embodiments, Y is CH.

In some embodiments, Y is CH. In some embodiments, Y is substituted with R⁴.

In some embodiments, Y is selected from those depicted in Table 1, below.

In some embodiments, Y is selected from those depicted in Table 1A, below.

In some embodiments, Y is selected from those depicted in Table 1B, below.

In some embodiments, Y is selected from those depicted in Table 1C, below.

As defined generally above, Z¹ is N, C═O, or CR².

In some embodiments, Z¹ is N. In some embodiments, Z¹ is C═O. In some embodiments, Z¹ is CR².

In some embodiments, Z¹ is selected from those depicted in Table 1, below.

In some embodiments, Z¹ is selected from those depicted in Table 1A, below.

In some embodiments, Z¹ is selected from those depicted in Table 1B, below.

In some embodiments, Z¹ is selected from those depicted in Table 1C, below.

As defined generally above, Z² is N, C═O, or CR³.

In some embodiments, Z² is N. In some embodiments, Z² is C═O. In some embodiments, Z² is CR³.

In some embodiments, Z² is selected from those depicted in Table 1, below.

In some embodiments, Z² is selected from those depicted in Table 1A, below.

In some embodiments, Z² is selected from those depicted in Table 1B, below.

In some embodiments, Z² is selected from those depicted in Table 1C, below.

As defined generally above, each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹ is R. In some embodiments, R¹ is halogen. In some embodiments, R¹ is —CN. In some embodiments, R¹ is —NC. In some embodiments, R¹ is —C(O)OR. In some embodiments, R¹ is —OC(O)R. In some embodiments, R¹ is —C(O)N(R)₂. In some embodiments, R¹ is —N(R)C(O)R. In some embodiments, R¹ is —N(R)C(O)N(R)₂. In some embodiments, R¹ is —OC(O)N(R)₂. In some embodiments, R¹ is —N(R)C(O)OR. In some embodiments, R¹ is —OR. In some embodiments, R¹ is —N(R)₂. In some embodiments, R¹ is —NO₂. In some embodiments, R¹ is —N₃. In some embodiments, R¹ is —SR. In some embodiments, R¹ is —S(O)R. In some embodiments, R¹ is —S(O)₂R. In some embodiments, R¹ is —S(O)₂N(R)₂. In some embodiments, R¹ is —NRS(O)₂R.

In some embodiments, R¹ is hydrogen. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, and sulfur. In some embodiments, R¹ is an optionally substituted 5-6 membered monocyclic heteroaromatic ring having 1-4 heteroatoms independently selected from nitrogen, oxygen, and sulfur. In some embodiments, R¹ is an optionally substituted 8-10 membered bicyclic heteroaromatic ring having 1-5 heteroatoms independently selected from nitrogen, oxygen, and sulfur.

In some embodiments, R¹ is selected from H, F, Cl, OH, Me, Et, i-Pr,

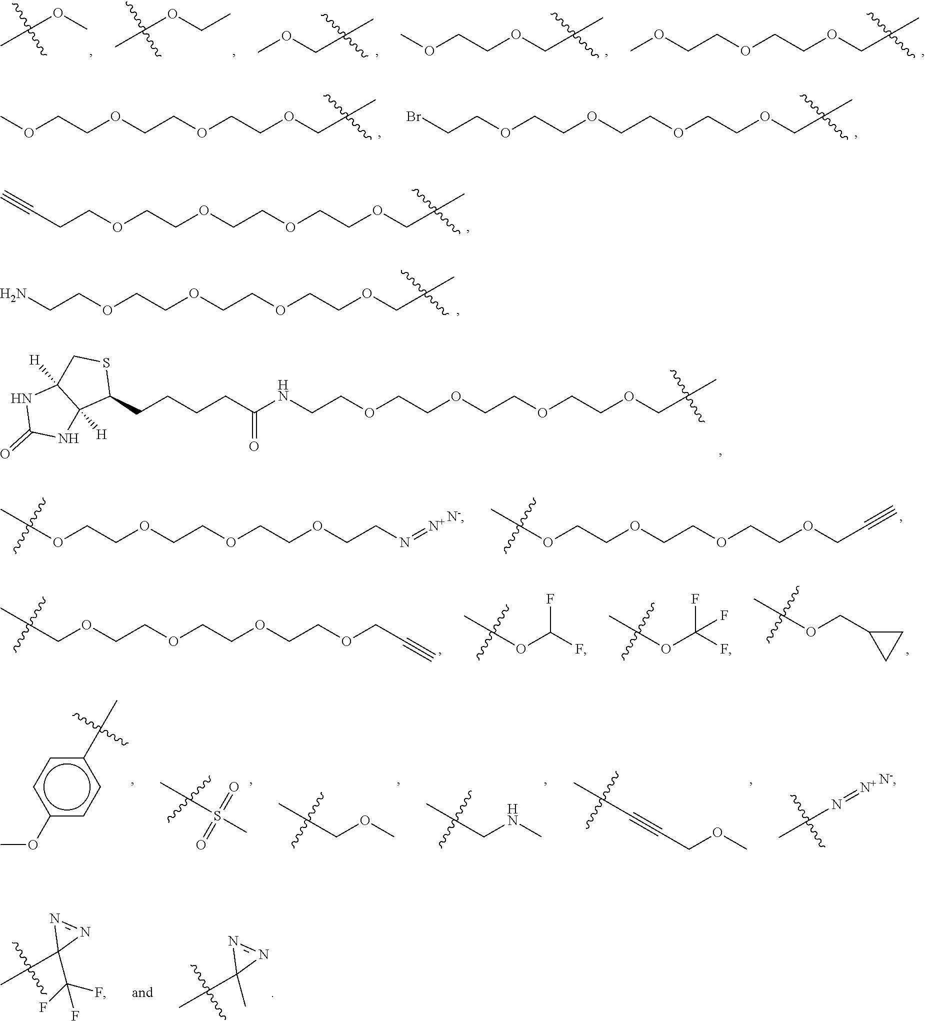

In some embodiments, R¹ is selected from H, F, Cl, OH, Me, Et, i-Pr,

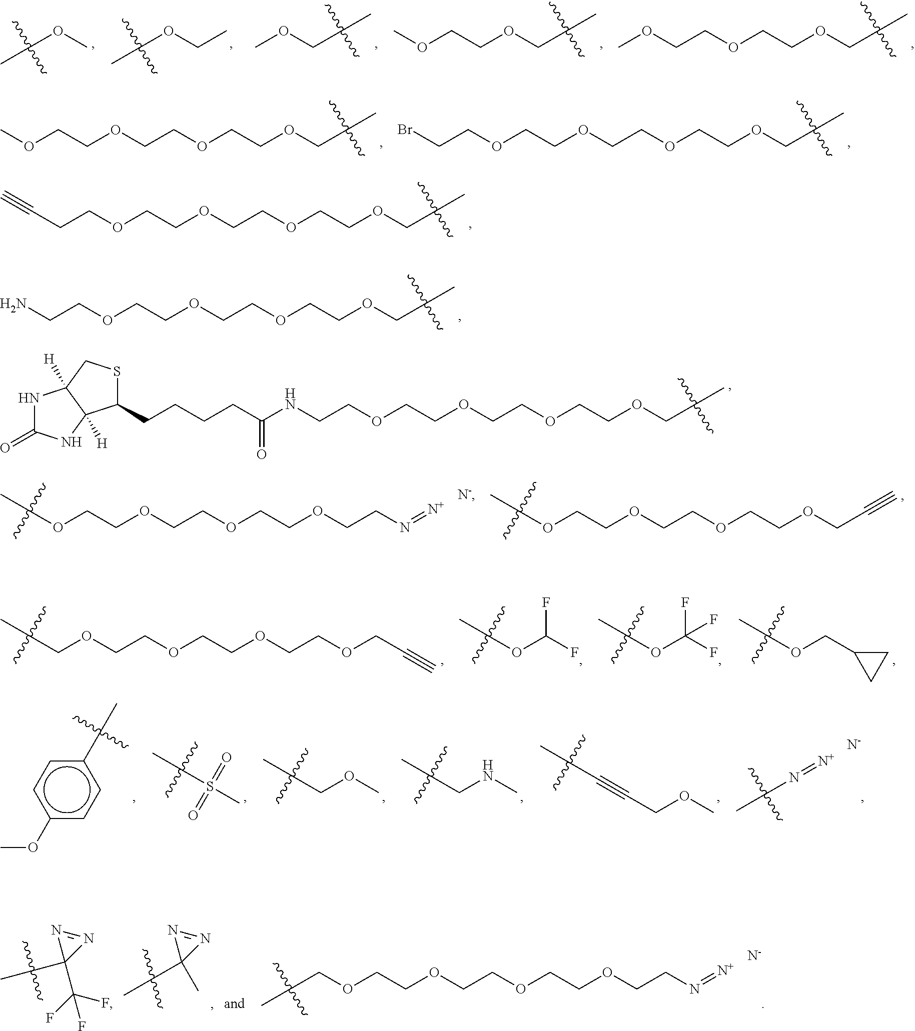

In some embodiments R¹ is selected from H, F, Cl, OH, Me Et, i-Pr

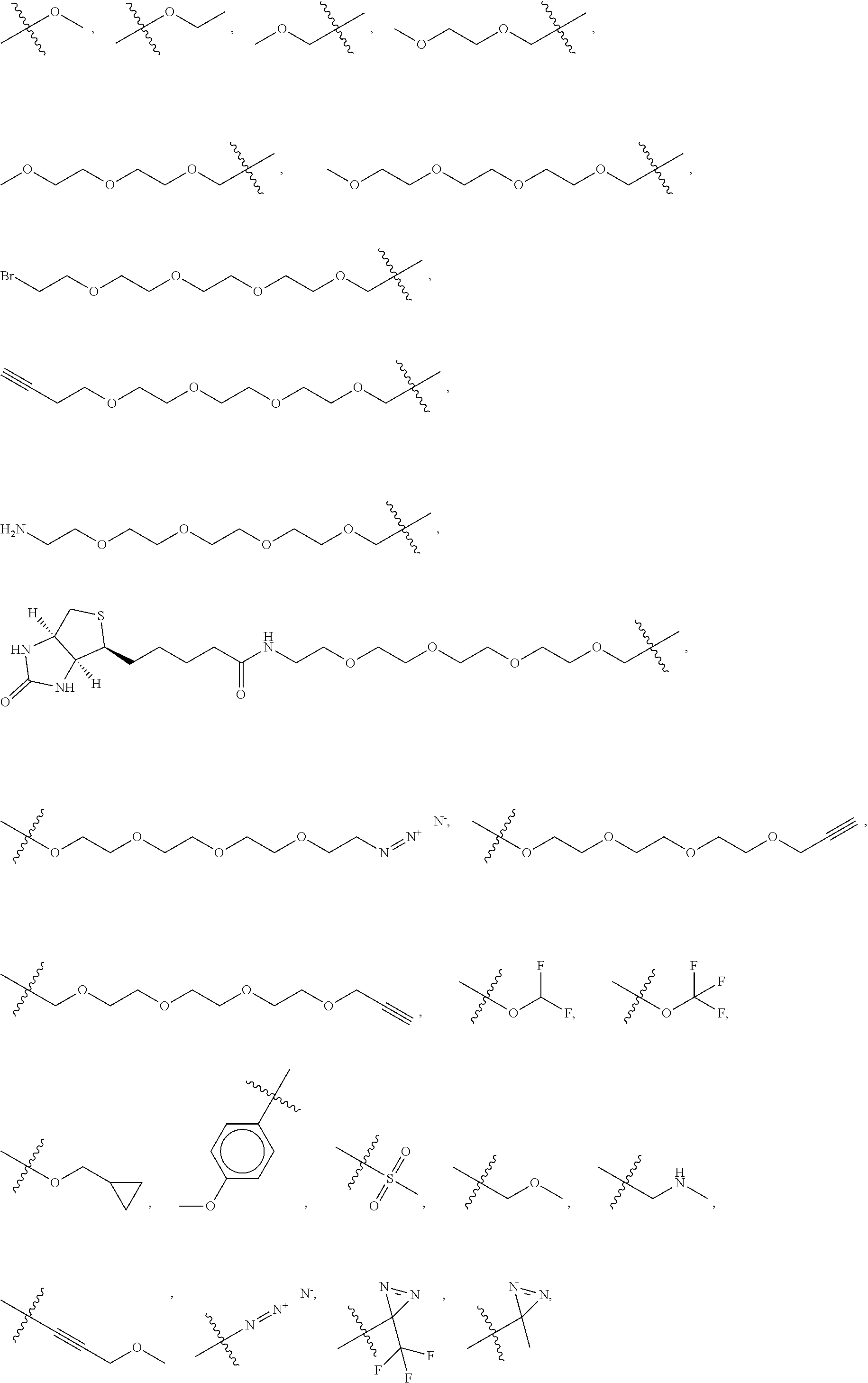

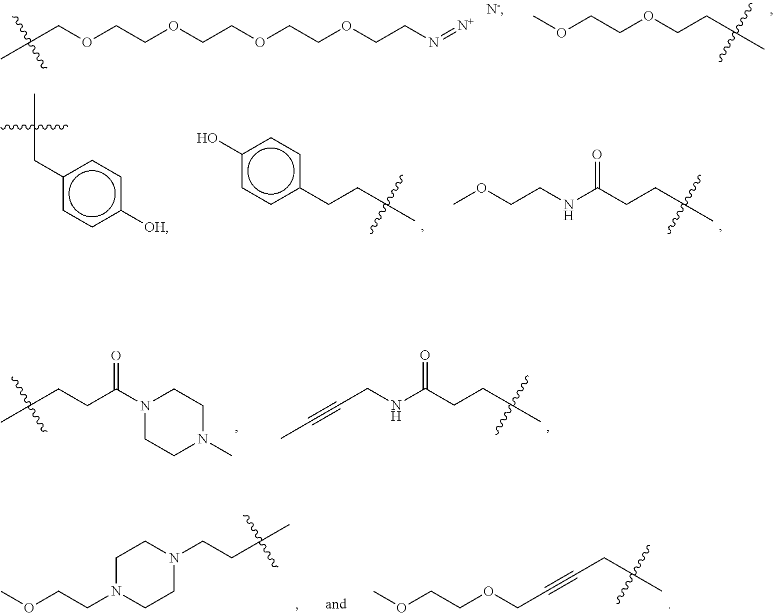

In some embodiments, R¹ is selected from those depicted in Table 1, below.

In some embodiments, R¹ is selected from those depicted in Table 1A, below.

In some embodiments, R¹ is selected from those depicted in Table 1B, below.

In some embodiments, R¹ is selected from those depicted in Table 1C, below.

As defined generally above, each R² and R³ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² is R. In some embodiments, R² is halogen. In some embodiments, R² is —CN. In some embodiments, R² is —NC. In some embodiments, R² is —C(O)OR. In some embodiments, R² is —OC(O)R. In some embodiments, R² is —C(O)N(R)₂. In some embodiments, R² is —N(R)C(O)R. In some embodiments, R² is —N(R)C(O)N(R)₂. In some embodiments, R² is —OC(O)N(R)₂. In some embodiments, R² is —N(R)C(O)OR. In some embodiments, R² is —OR. In some embodiments, R² is —N(R)₂. In some embodiments, R² is —NO₂. In some embodiments, R² is —N₃. In some embodiments, R² is —SR. In some embodiments, R² is —S(O)R. In some embodiments, R² is —S(O)₂R. In some embodiments, R² is —S(O)₂N(R)₂. In some embodiments, R² is —NRS(O)₂R.

In some embodiments, R² is hydrogen. In some embodiments, R² is an optionally substituted C₁₋₆ aliphatic group. In some embodiments, R² is an optionally substituted 4-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, and sulfur. In some embodiments, R² is an optionally substituted 5-6 membered monocyclic heteroaromatic ring having 1-4 heteroatoms independently selected from nitrogen, oxygen, and sulfur. In some embodiments, R² is an optionally substituted 8-10 membered bicyclic heteroaromatic ring having 1-5 heteroatoms independently selected from nitrogen, oxygen, and sulfur.

In some embodiments, R² is selected from H, F, Cl, OH, Me, Et, i-Pr,

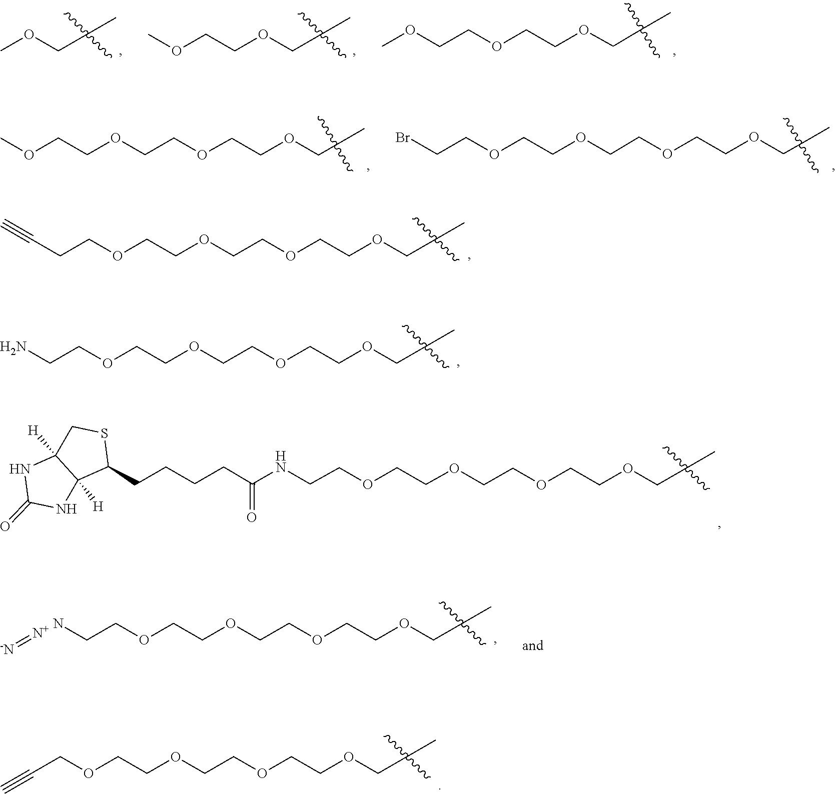

In some embodiments, R² is selected from H, F, Cl, OH, Me, Et, i-Pr,

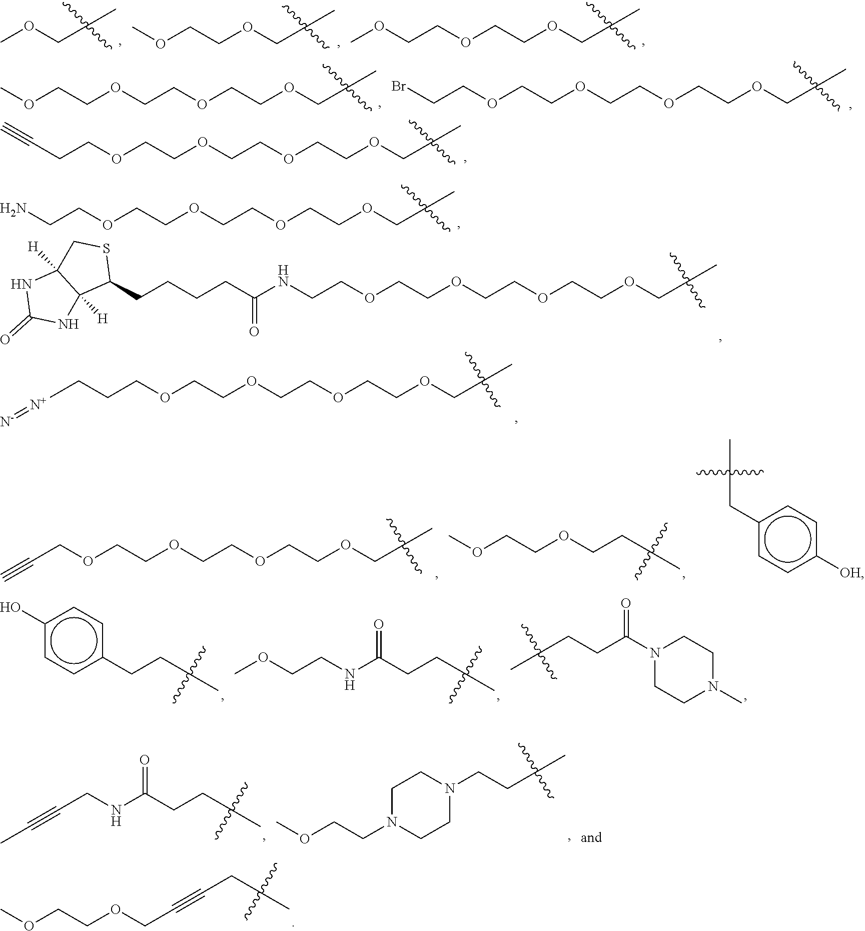

In some embodiments, R³ is R. In some embodiments, R³ is halogen. In some embodiments, R³ is —CN. In some embodiments, R³ is —NC. In some embodiments, R³ is —C(O)OR. In some embodiments, R³ is —OC(O)R. In some embodiments, R³ is —C(O)N(R)₂. In some embodiments, R³ is —N(R)C(O)R. In some embodiments, R³ is —N(R)C(O)N(R)₂. In some embodiments, R³ is —OC(O)N(R)₂. In some embodiments, R³ is —N(R)C(O)OR. In some embodiments, R³ is —OR. In some embodiments, R³ is —N(R)₂. In some embodiments, R³ is —NO₂. In some embodiments, R³ is —N₃. In some embodiments, R³ is —SR. In some embodiments, R³ is —S(O)R. In some embodiments, R³ is —S(O)₂R. In some embodiments, R³ is —S(O)₂N(R)₂. In some embodiments, R³ is —NRS(O)₂R.

In some embodiments, R³ is hydrogen. In some embodiments, R³ is an optionally substituted C₁₋₆ aliphatic group. In some embodiments, R³ is an optionally substituted 4-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, and sulfur. In some embodiments, R³ is an optionally substituted 5-6 membered monocyclic heteroaromatic ring having 1-4 heteroatoms independently selected from nitrogen, oxygen, and sulfur. In some embodiments, R³ is an optionally substituted 8-10 membered bicyclic heteroaromatic ring having 1-5 heteroatoms independently selected from nitrogen, oxygen, and sulfur.

In some embodiments, R³ is selected from H, F, Cl, OH, Me, Et, CF₃, CN, —C(O)NH₂, i-Pr,

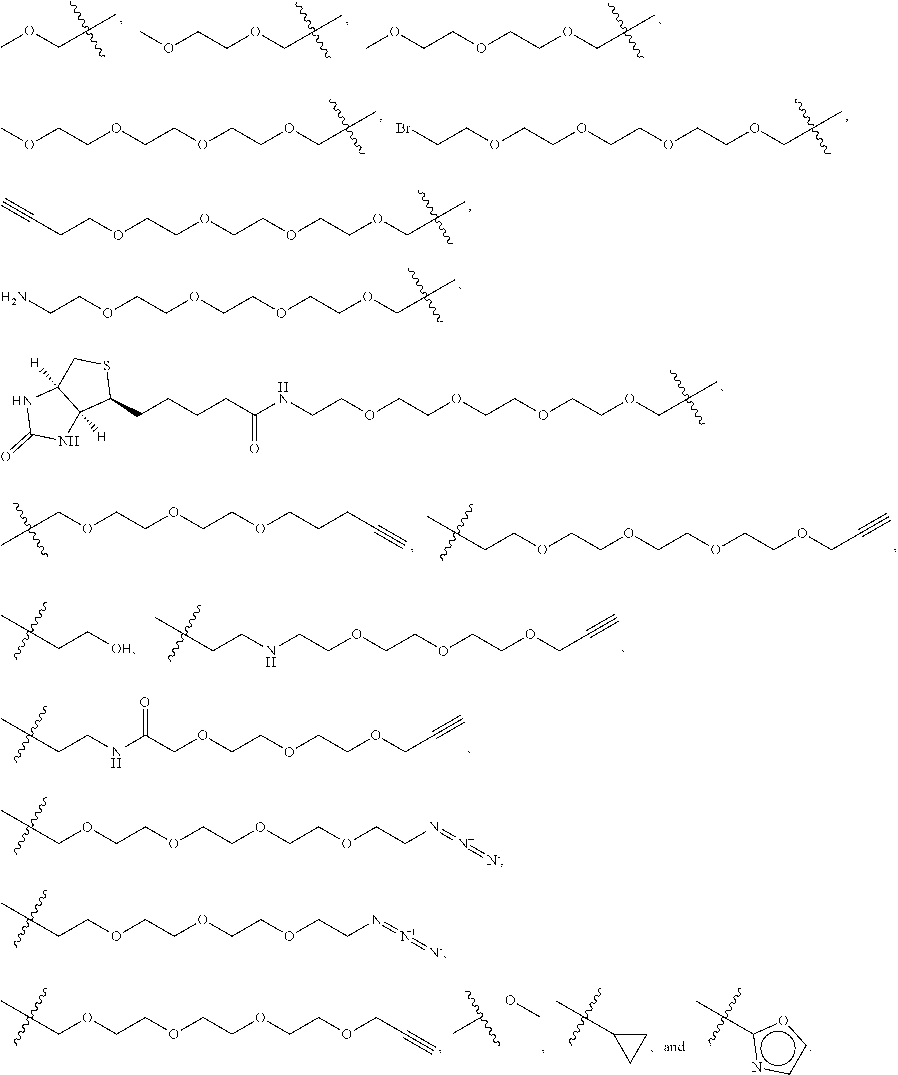

In some embodiments, R³ is selected from H, F, Cl, OH, Me, Et, CF₃, CN, —C(O)NH₂, i-Pr,

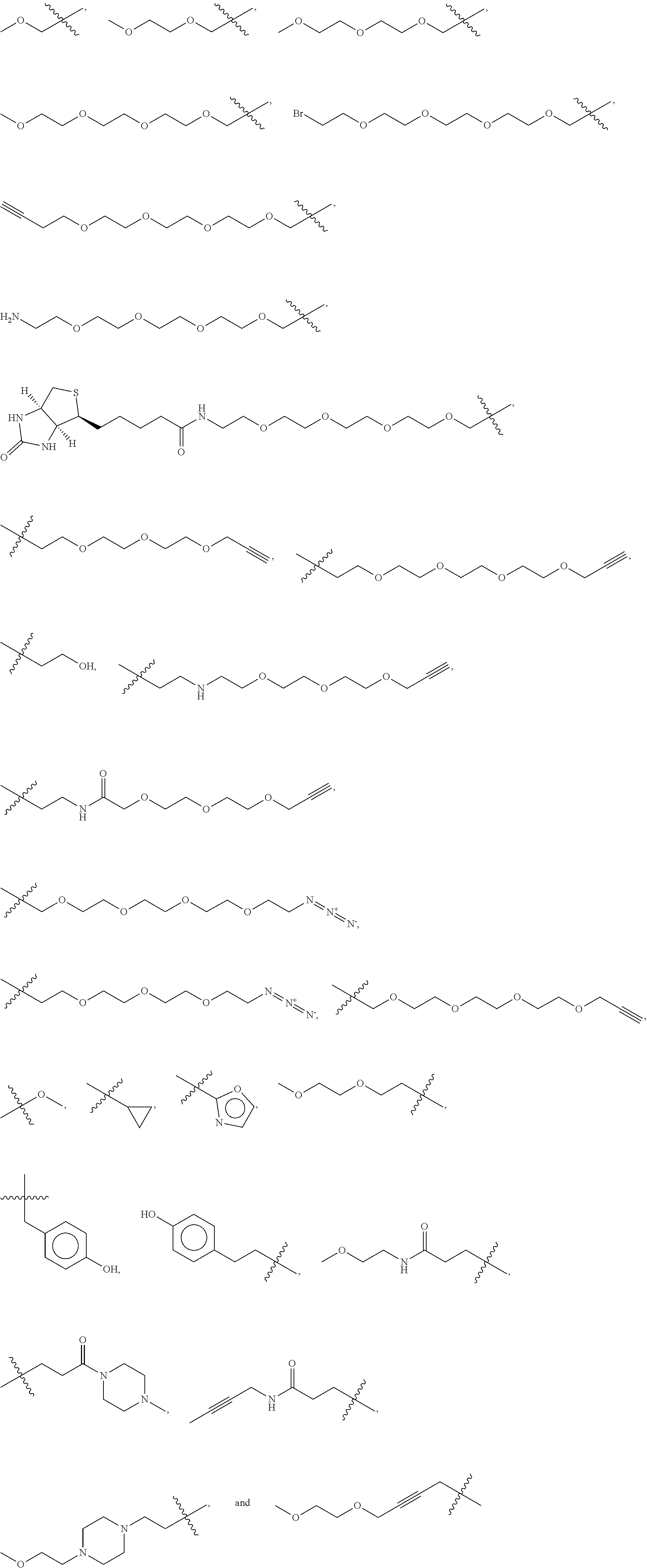

In some embodiments, R³ is selected from H, F, Cl, Me, and Et.

In some embodiments, R² and R³ taken together with the carbons to which they are attached form a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, R² and R³ taken together with the carbons to which they are attached form a 4-8 membered saturated or partially unsaturated monocyclic carbocyclic ring. In some embodiments, R² and R³ taken together with the carbons to which they are attached form phenyl. In some embodiments, R² and R³ taken together with the carbons to which they are attached form an 8-10 membered bicyclic aromatic carbocyclic ring. In some embodiments, R² and R³ taken together with the carbons to which they are attached form a 4-8 membered saturated or partially unsaturated monocyclic heterocyclic ring having 1-2 heteroatoms independently selected from nitrogen, oxygen, and sulfur. In some embodiments, R² and R³ taken together with the carbons to which they are attached form a 5-6 membered monocyclic heteroaromatic ring having 1-4 heteroatoms independently selected from nitrogen, oxygen, and sulfur. In some embodiments, R² and R³ taken together with the carbons to which they are attached form an 8-10 membered bicyclic heteroaromatic ring having 1-5 heteroatoms independently selected from nitrogen, oxygen, and sulfur.

In some embodiments, R² and R³ taken together with the carbons to which they are attached form a cyclohexane ring. In some embodiments, R² and R³ taken together with the carbons to which they are attached form a cyclopentane ring.

In some embodiments, R² and R³ are selected from those depicted in Table 1, below.

In some embodiments, R² and R³ are selected from those depicted in Table 1A, below.

In some embodiments, R² and R³ are selected from those depicted in Table 1B, below.

In some embodiments, R² and R³ are selected from those depicted in Table 1C, below.

As defined generally above, each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R⁴ is R. In some embodiments, R⁴ is halogen. In some embodiments, R⁴ is ═O. In some embodiments, R⁴ is —CN. In some embodiments, R⁴ is —NC. In some embodiments, R⁴ is —C(O)OR. In some embodiments, R⁴ is —OC(O)R. In some embodiments, R⁴ is —C(O)N(R)₂. In some embodiments, R⁴ is —N(R)C(O)R. In some embodiments, R⁴ is —N(R)C(O)N(R)₂. In some embodiments, R⁴ is —OC(O)N(R)₂. In some embodiments, R⁴ is —N(R)C(O)OR In some embodiments, R⁴ is —OR. In some embodiments, R⁴ is —N(R)₂. In some embodiments, R⁴ is —NO₂. In some embodiments, R⁴ is —N₃. In some embodiments, R⁴ is —SR. In some embodiments, R⁴ is —S(O)R. In some embodiments, R⁴ is —S(O)₂R. In some embodiments, R⁴ is —S(O)₂N(R)₂. In some embodiments, R⁴ is —NRS(O)₂R. In some embodiments, R⁴ is C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R⁴ is hydrogen. In some embodiments, R⁴ is an optionally substituted C₁₋₆ aliphatic group. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 4-8 membered saturated or partially unsaturated monocyclic heterocyclic ring having 1-2 heteroatoms independently selected from nitrogen, oxygen, and sulfur. In some embodiments, R⁴ is an optionally substituted 5-6 membered monocyclic heteroaromatic ring having 1-4 heteroatoms independently selected from nitrogen, oxygen, and sulfur. In some embodiments, R⁴ is an optionally substituted 8-10 membered bicyclic heteroaromatic ring having 1-5 heteroatoms independently selected from nitrogen, oxygen, and sulfur.

In some embodiments, R⁴ is selected from H, ═O, Me, Et, iPr,

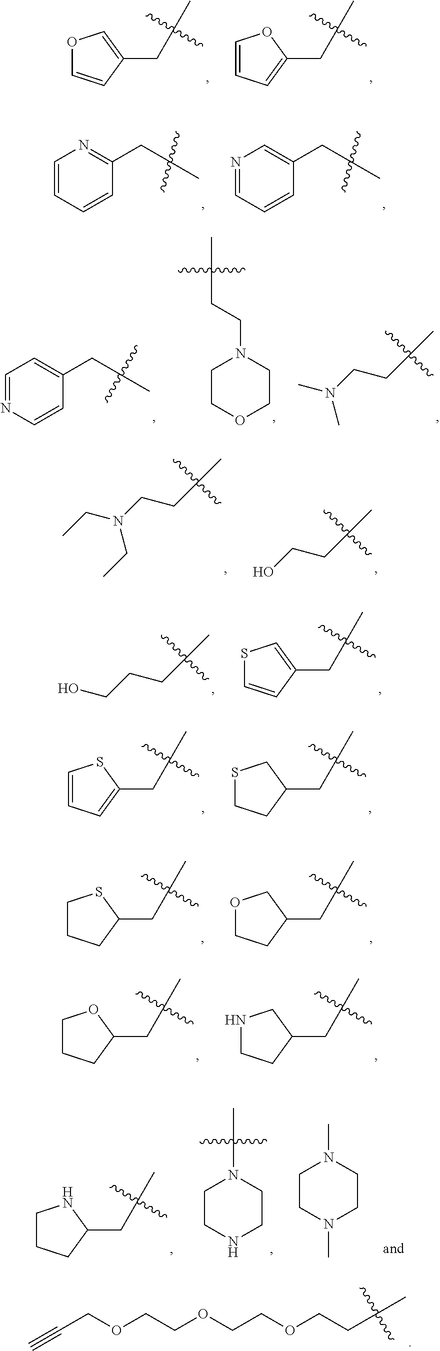

In some embodiments, R⁴ is selected from H, ═O, Me, Et, iPr,

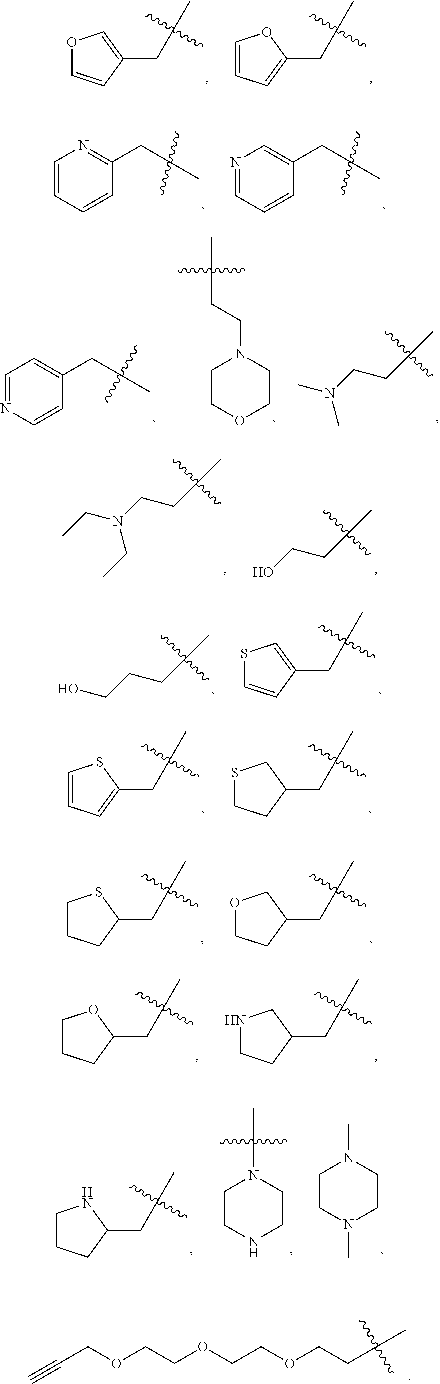

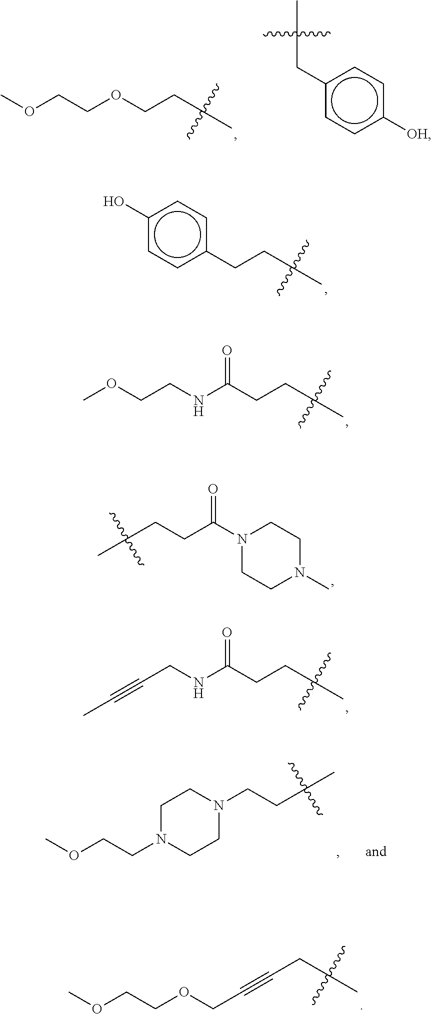

In some embodiments, R⁴ is selected from those depicted in Table 1, below.

In some embodiments, R⁴ is selected from those depicted in Table 1A, below.

In some embodiments, R⁴ is selected from those depicted in Table 1B, below.

In some embodiments, R⁴ is selected from those depicted in Table 1C, below.

As defined generally above, each R⁵ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R⁵ is R. In some embodiments, R⁵ is halogen. In some embodiments, R⁵ is —CN. In some embodiments, R⁵ is —NC. In some embodiments, R⁵ is —C(O)OR. In some embodiments, R⁵ is —OC(O)R. In some embodiments, R⁵ is —C(O)N(R)₂. In some embodiments, R⁵ is —N(R)C(O)R. In some embodiments, R⁵ is —N(R)C(O)N(R)₂. In some embodiments, R⁵ is —OC(O)N(R)₂. In some embodiments, R⁵ is —N(R)C(O)OR. In some embodiments, R⁵ is —OR. In some embodiments, R⁵ is —N(R)₂. In some embodiments, R⁵ is —NO₂. In some embodiments, R⁵ is —N₃. In some embodiments, R⁵ is —SR. In some embodiments, R⁵ is —S(O)R. In some embodiments, R⁵ is —S(O)₂R. In some embodiments, R⁵ is —S(O)₂N(R)₂. In some embodiments, R⁵ is —NRS(O)₂R.

In some embodiments, R⁵ is selected from those depicted in Table 1, below.

In some embodiments, R⁵ is selected from those depicted in Table 1A, below.

In some embodiments, R⁵ is selected from those depicted in Table 1B, below.

In some embodiments, R⁵ is selected from those depicted in Table 1C, below.

As defined generally above, -L²- is

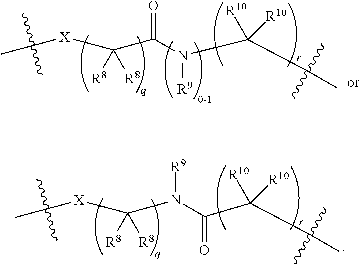

In some embodiments, -L²- is

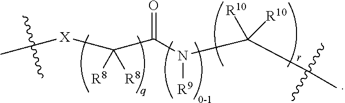

In some embodiments, -L²- is

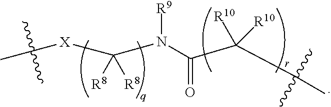

As defined generally above, each R⁶ and R⁷ is independently —R, halogen, —CN, —NC, —C(O)OR, —OC(O)R, —OR, —N(R)₂, or —SR.

In some embodiments, R⁶ is R. In some embodiments, R⁶ is halogen. In some embodiments, R⁶ is —CN. In some embodiments, R⁶ is —NC. In some embodiments, R⁶ is —C(O)OR. In some embodiments, R⁶ is —OC(O)R. In some embodiments, R⁶ is —OR. In some embodiments, R⁶ is —N(R)₂. In some embodiments, R⁶ is —SR.

In some embodiments, R⁶ is hydrogen. In some embodiments, R⁶ is an optionally substituted C₁₋₆ aliphatic group. In some embodiments, R⁶ is an optionally substituted 3-8 membered saturated or partially unsaturated monocyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 4-8 membered saturated or partially unsaturated monocyclic heterocyclic ring having 1-2 heteroatoms independently selected from nitrogen, oxygen, and sulfur. In some embodiments, R⁶ is an optionally substituted 5-6 membered monocyclic heteroaromatic ring having 1-4 heteroatoms independently selected from nitrogen, oxygen, and sulfur. In some embodiments, R⁶ is an optionally substituted 8-10 membered bicyclic heteroaromatic ring having 1-5 heteroatoms independently selected from nitrogen, oxygen, and sulfur.

In some embodiments, R⁶ is selected from H, Me, Et, iPr, F, and OH.

In some embodiments, R⁶ is selected from those depicted in Table 1, below.

In some embodiments, R⁶ is selected from those depicted in Table 1A, below.

In some embodiments, R⁶ is selected from those depicted in Table 1B, below.

In some embodiments, R⁶ is selected from those depicted in Table 1C, below.

In some embodiments, R⁷ is R. In some embodiments, R⁷ is halogen. In some embodiments, R⁷ is —CN. In some embodiments, R⁷ is —NC. In some embodiments, R⁷ is —C(O)OR. In some embodiments, R⁷ is —OC(O)R. In some embodiments, R⁷ is —OR. In some embodiments, R⁷ is —N(R)₂. In some embodiments, R⁷ is —SR.

In some embodiments, R⁷ is hydrogen. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, and sulfur. In some embodiments, R⁷ is an optionally substituted 5-6 membered monocyclic heteroaromatic ring having 1-4 heteroatoms independently selected from nitrogen, oxygen, and sulfur. In some embodiments, R⁷ is an optionally substituted 8-10 membered bicyclic heteroaromatic ring having 1-5 heteroatoms independently selected from nitrogen, oxygen, and sulfur.

In some embodiments, R⁷ is selected from H, Me, Et, iPr, F and OH.

In some embodiments, R⁷ is selected from those depicted in Table 1, below.

In some embodiments, R⁷ is selected from those depicted in Table 1A, below.

In some embodiments, R⁷ is selected from those depicted in Table 1B, below.

In some embodiments, R⁷ is selected from those depicted in Table 1C, below.

As defined generally above, X is a bond, NR⁶, O, CR⁶R⁷—C(O)—, —S—, or —S(O)₂—.

In some embodiments, X is selected from a bond, NH, O, S, SO₂, C(O), NMe, CF₂, CHMe, CHOH, C(Me)₂ and C(Me)OH.

In some embodiments, X is selected from those depicted in Table 1, below.

In some embodiments, X is selected from those depicted in Table 1A, below.

In some embodiments, X is selected from those depicted in Table 1B, below.

In some embodiments, X is selected from those depicted in Table 1C, below.

As defined generally above, each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or two R⁸ taken together with the carbon atom to which they are attached, form a 3-6 membered carbocyclic ring.

In some embodiments, R⁸ is R. In some embodiments, R⁸ is halogen. In some embodiments, R⁸ is —CN. In some embodiments, R⁸ is —NC. In some embodiments, R⁸ is —C(O)OR. In some embodiments, R⁸ is —OC(O)R. In some embodiments, R⁸ is —C(O)N(R)₂. In some embodiments, R⁸ is —N(R)C(O)R. In some embodiments, R⁸ is —N(R)C(O)N(R)₂. In some embodiments, R⁸ is —OC(O)N(R)₂. In some embodiments, R⁸ is —N(R)C(O)OR. In some embodiments, R⁸ is —OR. In some embodiments, R⁸ is —N(R)₂. In some embodiments, R⁸ is —NO₂. In some embodiments, R⁸ is —N₃. In some embodiments, R⁸ is —SR. In some embodiments, R⁸ is —S(O)R. In some embodiments, R⁸ is —S(O)₂R. In some embodiments, R⁸ is —S(O)₂N(R)₂. In some embodiments, R⁸ is —NRS(O)₂R. In some embodiments, two R⁸ taken together with the carbon atom to which they are attached, form a 3-6 membered carbocyclic ring.

In some embodiments, R⁸ is hydrogen. In some embodiments, R⁸ is an optionally substituted C₁₋₆ aliphatic group. In some embodiments, R⁸ is an optionally substituted 3-8 membered saturated or partially unsaturated monocyclic carbocyclic ring. In some embodiments, R⁸ is an optionally substituted phenyl. In some embodiments, R⁹ is an optionally substituted 8-10 membered bicyclic aromatic carbocyclic ring. In some embodiments, R⁸ is an optionally substituted 4-8 membered saturated or partially unsaturated monocyclic heterocyclic ring having 1-2 heteroatoms independently selected from nitrogen, oxygen, and sulfur. In some embodiments, R⁸ is an optionally substituted 5-6 membered monocyclic heteroaromatic ring having 1-4 heteroatoms independently selected from nitrogen, oxygen, and sulfur. In some embodiments, R⁸ is an optionally substituted 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R⁸)₂— is optionally replaced by a 3-8 membered saturated or partially unsaturated monocyclic carbocyclic ring. In some embodiments, one instance of —C(R⁸)₂— is optionally replaced by phenyl. In some embodiments, one instance of —C(R⁸)₂— is optionally replaced by an 8-10 membered bicyclic aromatic carbocyclic ring. In some embodiments, one instance of —C(R⁸)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R⁸)₂— is optionally replaced by a 5-6 membered monocyclic heteroaromatic ring having 1-4 heteroatoms independently selected from nitrogen, oxygen, and sulfur. In some embodiments, one instance of —C(R⁸)₂— is optionally replaced by an 8-10 membered bicyclic heteroaromatic ring having 1-5 heteroatoms independently selected from nitrogen, oxygen, and sulfur.

In some embodiments, one instance of —C(R⁸)₂— is optionally replaced by

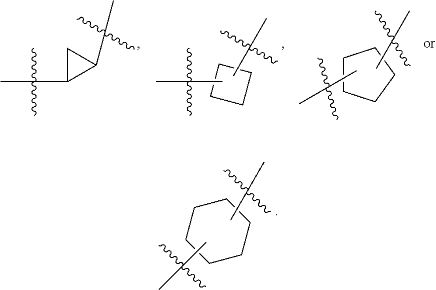

In some embodiments, R⁸ is selected from H, F, Cl, OH, Me, Et, i-Pr,

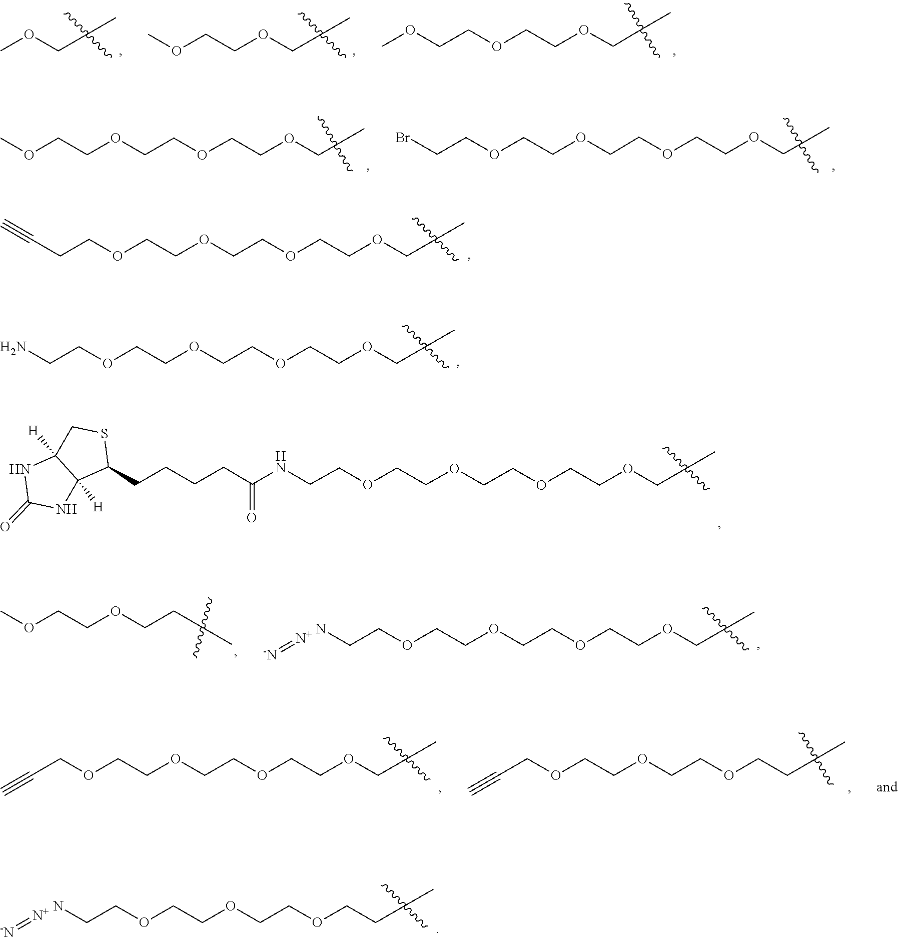

In some embodiments, R⁸ is selected from H, F, Cl, OH, Me, Et, i-Pr,

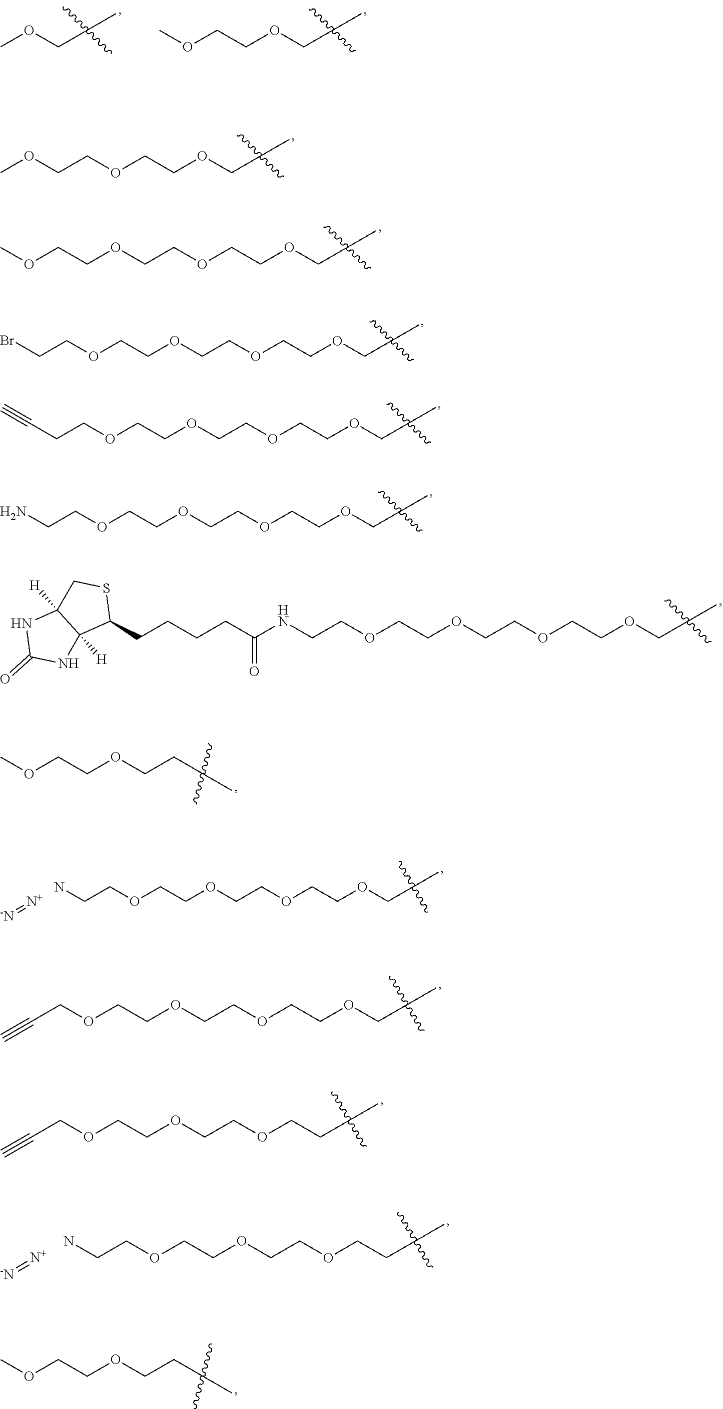

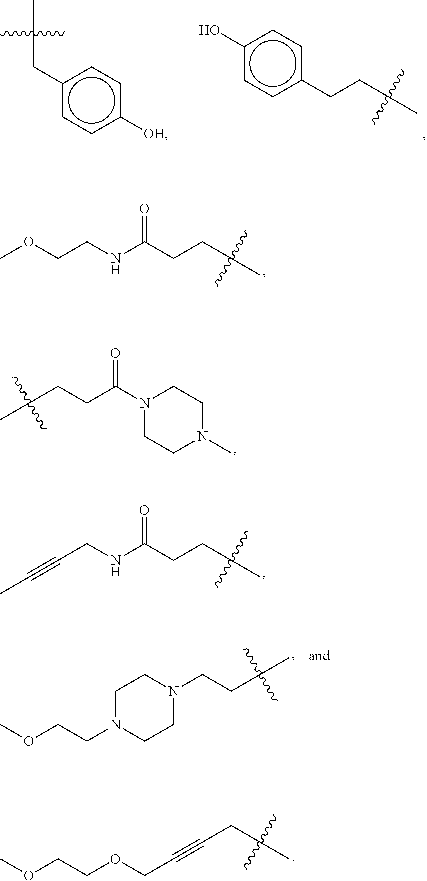

In some embodiments, R⁸ is selected from H, F, Cl, OH, Me, Et, i-Pr,

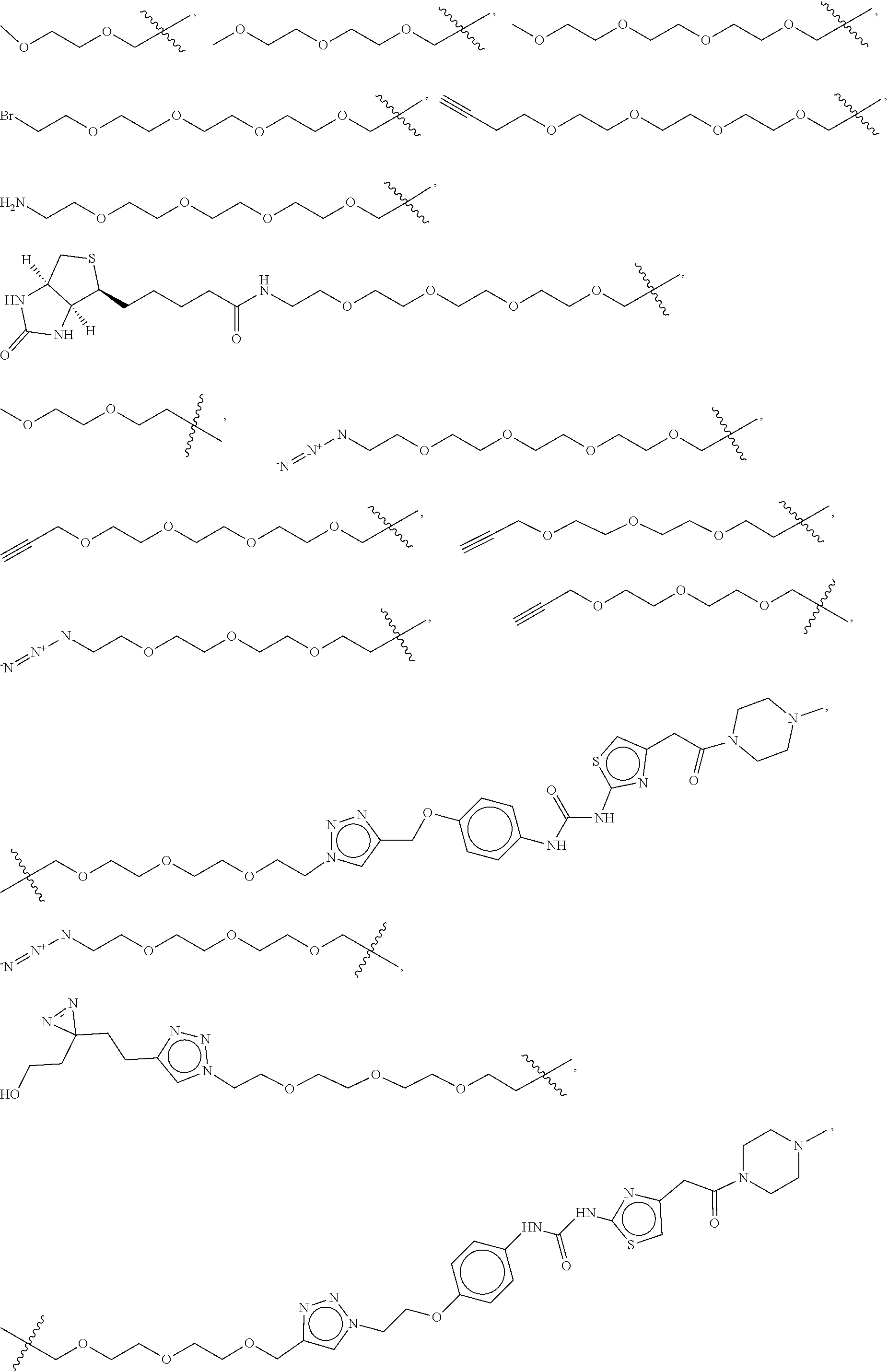

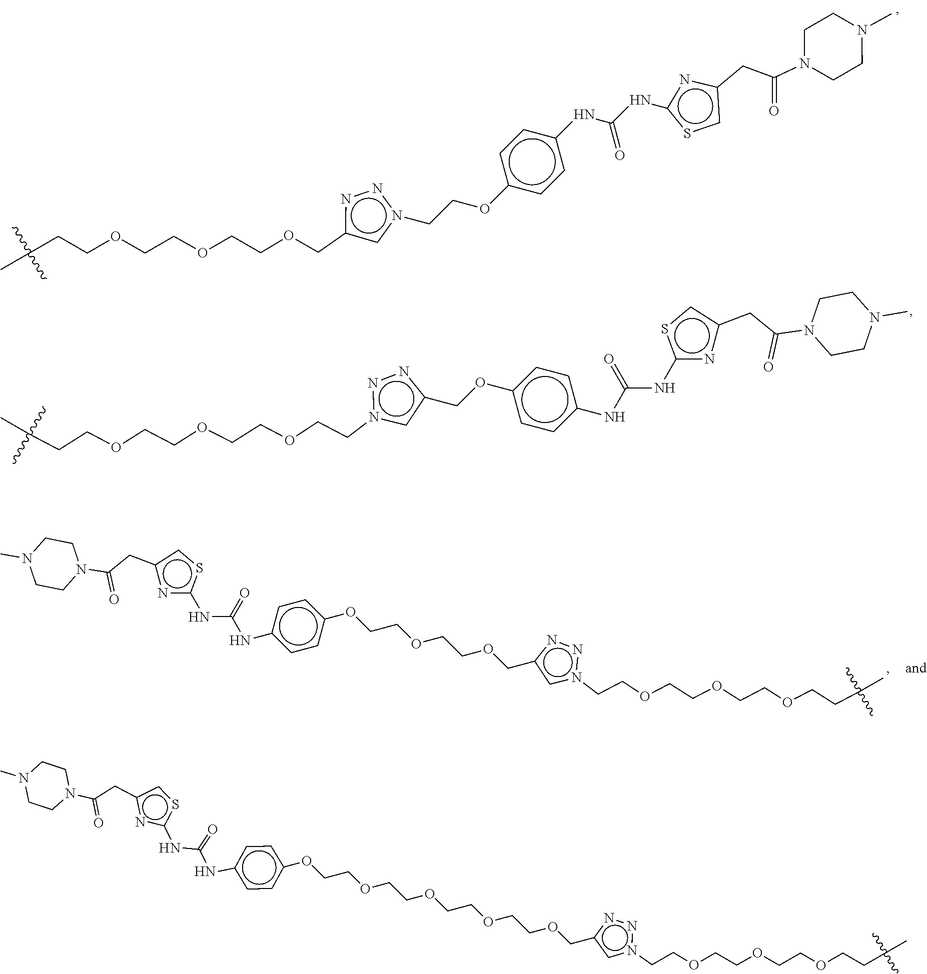

In some embodiments, two R⁸ taken together with the carbon atom to which they are attached, form a cyclopropane ring.

In some embodiments, two R⁸ taken together with the carbon atom to which they are attached, form a cyclobutane ring.

In some embodiments, R⁸ is selected from those depicted in Table 1, below.

In some embodiments, R⁸ is selected from those depicted in Table 1A, below.

In some embodiments, R⁸ is selected from those depicted in Table 1B, below.

In some embodiments, R⁸ is selected from those depicted in Table 1C, below.

As defined generally above, R⁹ is —R, halogen, —CN, —NC, —C(O)OR, —OC(O)R, —OR, —N(R)₂, or —SR.

In some embodiments, R⁹ is R. In some embodiments, R⁹ is halogen. In some embodiments, R⁹ is —CN. In some embodiments, R⁹ is —NC. In some embodiments, R⁹ is —C(O)OR. In some embodiments, R⁹ is —OC(O)R. In some embodiments, R⁹ is —OR. In some embodiments, R⁹ is —N(R)₂. In some embodiments, R⁹ is —SR.

In some embodiments, R⁹ is hydrogen. In some embodiments, R⁹ is an optionally substituted C₁₋₆ aliphatic group. In some embodiments, R⁹ is an optionally substituted 3-8 membered saturated or partially unsaturated monocyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 4-8 membered saturated or partially unsaturated monocyclic heterocyclic ring having 1-2 heteroatoms independently selected from nitrogen, oxygen, and sulfur. In some embodiments, R⁹ is an optionally substituted 5-6 membered monocyclic heteroaromatic ring having 1-4 heteroatoms independently selected from nitrogen, oxygen, and sulfur. In some embodiments, R⁹ is an optionally substituted 8-10 membered bicyclic heteroaromatic ring having 1-5 heteroatoms independently selected from nitrogen, oxygen, and sulfur.

In some embodiments, R⁹ is selected from H, Me, Et, iPr,

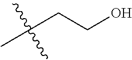 [SMILES: *CCO]

### and OH.

In some embodiments, R⁹ is selected from those depicted in Table 1, below.

In some embodiments, R⁹ is selected from those depicted in Table 1A, below.

In some embodiments, R⁹ is selected from those depicted in Table 1B, below.

In some embodiments, R⁹ is selected from those depicted in Table 1C, below.

In some embodiments, R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur.

As defined generally above, R¹⁰ is —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R.

In some embodiments, R¹⁰ is R. In some embodiments, R¹⁰ is halogen. In some embodiments, R¹⁰ is —CN. In some embodiments, R¹⁰ is —NC. In some embodiments, R¹⁰ is —C(O)OR. In some embodiments, R¹⁰ is —OC(O)R. In some embodiments, R¹⁰ is —C(O)N(R)₂. In some embodiments, R¹⁰ is —N(R)C(O)R. In some embodiments, R¹⁰ is —N(R)C(O)N(R)₂. In some embodiments, R¹⁰ is —OC(O)N(R)₂. In some embodiments, R¹⁰ is —N(R)C(O)OR. In some embodiments, R¹⁰ is —OR. In some embodiments, R¹⁰ is —N(R)₂. In some embodiments, R¹⁰ is —NO₂. In some embodiments, R¹⁰ is —N₃. In some embodiments, R¹⁰ is —SR. In some embodiments, R¹⁰ is —S(O)R. In some embodiments, R¹⁰ is —S(O)₂R. In some embodiments, R¹⁰ is —S(O)₂N(R)₂. In some embodiments, R¹⁰ is —NRS(O)₂R.

In some embodiments, R¹⁰ is hydrogen. In some embodiments, R¹⁰ is an optionally substituted C₁₋₆ aliphatic group. In some embodiments, R¹⁰ is an optionally substituted 3-8 membered saturated or partially unsaturated monocyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 4-8 membered saturated or partially unsaturated monocyclic heterocyclic ring having 1-2 heteroatoms independently selected from nitrogen, oxygen, and sulfur. In some embodiments, R¹⁰ is an optionally substituted 5-6 membered monocyclic heteroaromatic ring having 1-4 heteroatoms independently selected from nitrogen, oxygen, and sulfur. In some embodiments, R¹⁰ is an optionally substituted 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur.

In some embodiments, one instance of —C(R¹⁰)₂— is optionally replaced by a 3-8 membered saturated or partially unsaturated monocyclic carbocyclic ring. In some embodiments, one instance of —C(R¹⁰)₂— is optionally replaced by phenyl. In some embodiments, one instance of —C(R¹⁰)₂— is optionally replaced by an 8-10 membered bicyclic aromatic carbocyclic ring. In some embodiments, one instance of —C(R¹⁰)₂— is optionally replaced by a 4-8 membered saturated or partially unsaturated monocyclic heterocyclic ring having 1-3 heteroatoms independently selected from nitrogen, oxygen, and sulfur. In some embodiments, one instance of —C(R¹⁰)₂— is optionally replaced by a 5-6 membered monocyclic heteroaromatic ring having 1-4 heteroatoms independently selected from nitrogen, oxygen, and sulfur. In some embodiments, one instance of —C(R¹⁰)₂— is optionally replaced by an 8-10 membered bicyclic heteroaromatic ring having 1-5 heteroatoms independently selected from nitrogen, oxygen, and sulfur.

In some embodiments, one instance of —C(R¹⁰)₂— is optionally replaced by

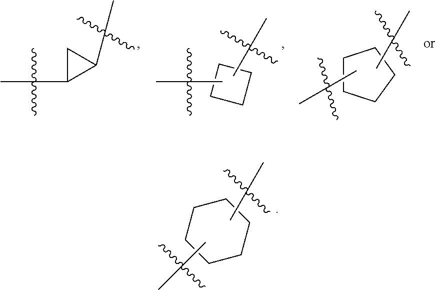

In some embodiments, two R¹⁰ taken together with the carbon atom to which they are attached, form a cyclopropane ring.

In some embodiments, R¹⁰ is selected from H, Me, Et, iPr,

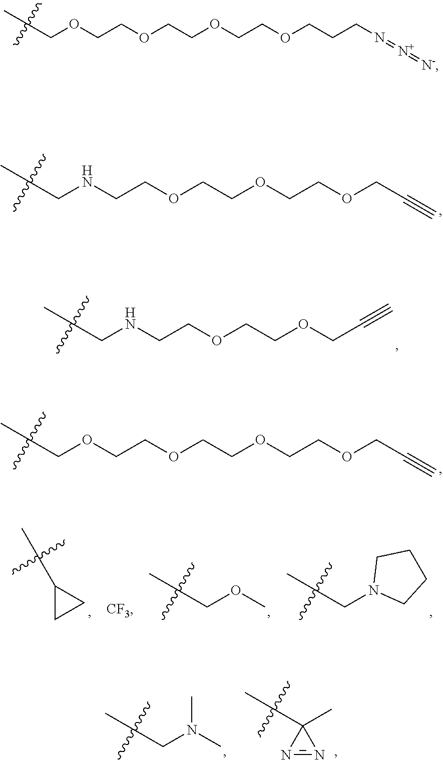

### and CH₃OH.

In some embodiments, R¹⁰ is selected from H, Me, Et, iPr,

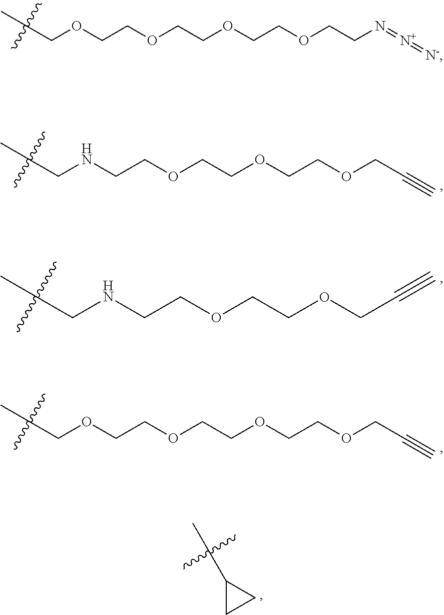

### CF₃,

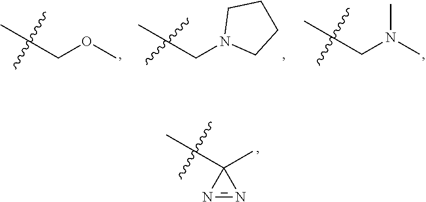

### CH₃OH, and

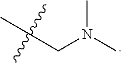 [SMILES: *CN(C)C]

In some embodiments, R¹⁰ is selected from those depicted in Table 1, below.

In some embodiments, R¹⁰ is selected from those depicted in Table 1A, below.

In some embodiments, R¹⁰ is selected from those depicted in Table 1B, below.

In some embodiments, R¹⁰ is selected from those depicted in Table 1C, below.

In some embodiments, R⁹ and R¹⁰ taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur.

In some embodiments, R⁹ and R¹⁰ taken together with the atoms to which they are attached, form a ring substituted with m instances of R¹; wherein the ring is a 4-8 membered saturated or partially unsaturated monocyclic heterocyclic ring having 1-2 heteroatoms independently selected from nitrogen, oxygen, and sulfur.

As defined generally above, R¹¹ is H, or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R¹¹ is H. In some embodiments, R¹¹ is C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—.

In some embodiments, R¹¹ is selected from H, Me, Et, iPr,

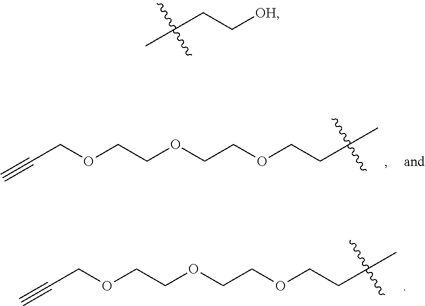

In some embodiments, R¹¹ is selected from H, Me, Et, iPr,

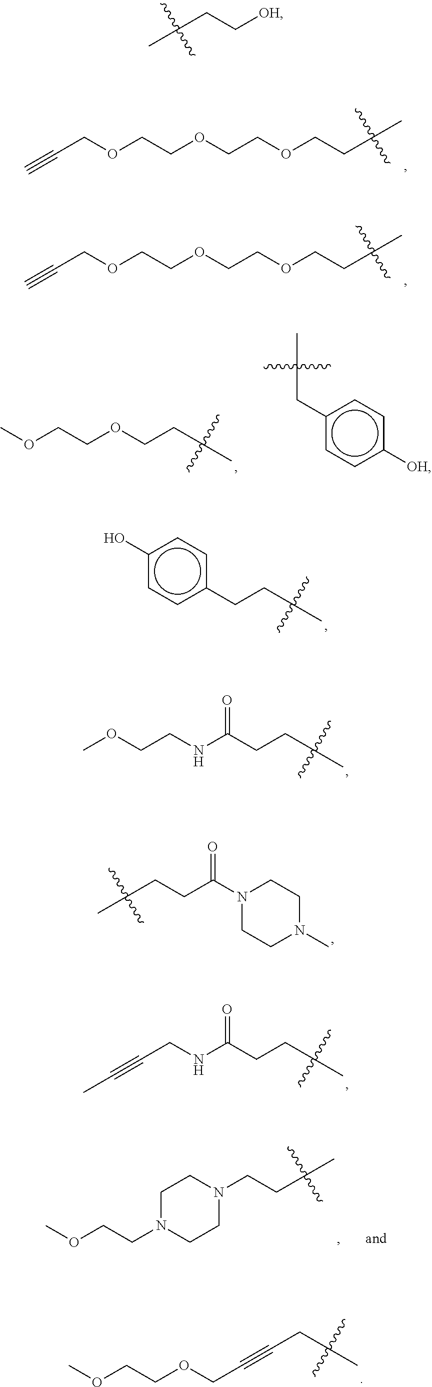

In some embodiments, R¹¹ is selected from those depicted in Table 1A, below.

In some embodiments, R¹¹ is selected from those depicted in Table 1B, below.

In some embodiments, R¹¹ is selected from those depicted in Table 1C, below.

In some embodiments, -L²- is selected from

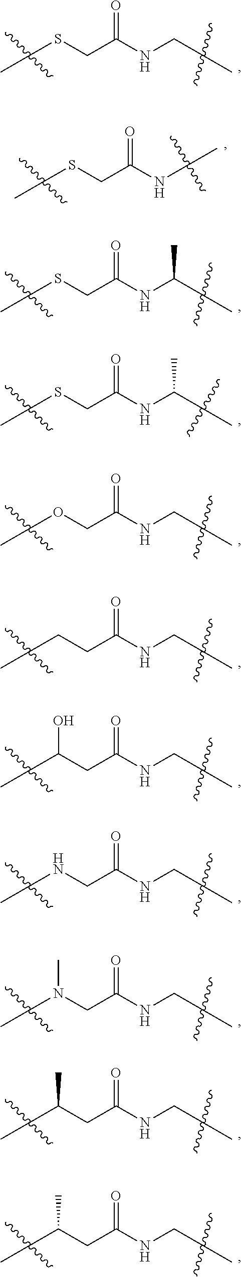

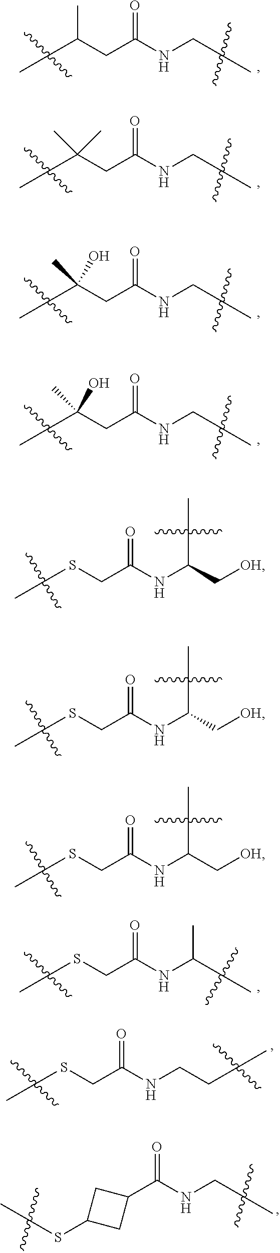

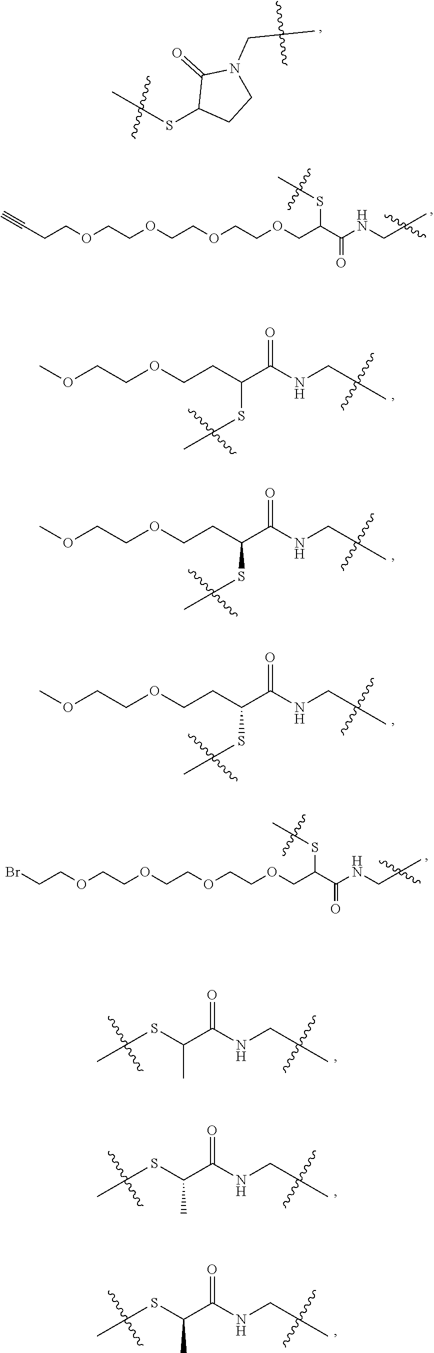

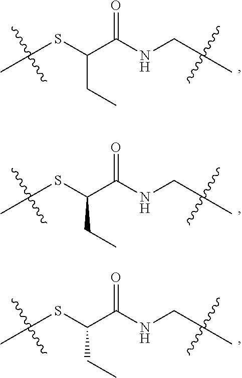

In some embodiments, -L²- is selected from those depicted in Table 1, below.

In some embodiments, -L²- is selected from those depicted in Table 1A, below.

In some embodiments, -L²- is selected from those depicted in Table 1B, below.

In some embodiments, -L²- is selected from those depicted in Table 1C, below.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0, 1, 2, or 3. In some embodiments, m is 0, 1, or 2. In some embodiments, m is 1, 2, or 3.

As defined generally above, n is 0, 1, 2, 3, 4 or 5. In some embodiments, n is 0. In some embodiments, n is 1. In some embodiments, n is 2. In some embodiments, n is 3. In some embodiments, n is 4. In some embodiments, n is 5. In some embodiments, n is 0, 1, 2, 3 or 4. In some embodiments, n is 0, 1, 2, or 3. In some embodiments, n is 0, 1, or 2. In some embodiments,
n is 1, 2, or 3.

As defined generally above, p is 0, 1, 2, or 3. In some embodiments, p is 0. In some embodiments, p is 1. In some embodiments, p is 2. In some embodiments, p is 3. In some embodiments, p is 0, 1, 2, or 3. In some embodiments, p is 0, 1, or 2. In some embodiments, p is 1, 2, or 3.

As defined generally above, q is 0, 1, 2, 3, or 4. In some embodiments, q is 0. In some embodiments, q is 1. In some embodiments, q is 2. In some embodiments, q is 3. In some embodiments, q is 4. In some embodiments, q is 0, 1, 2, or 3. In some embodiments, q is 0, 1, or 2. In some embodiments, q is 1, 2, or 3.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0, 1, 2, or 3. In some embodiments, r is 0, 1, or 2. In some embodiments, r is 1, 2, or 3.

Exemplary DFL compounds of the invention are set forth in Table 1, below.

Exemplary DFL compounds of the invention are set forth in Table 1A, below.

Exemplary DFL compounds of the invention are set forth in Table 1B, below.

Exemplary DFL compounds of the invention are set forth in Table 1C, below.

In some embodiments, the present invention provides a compound of Formula II-c or II-d:

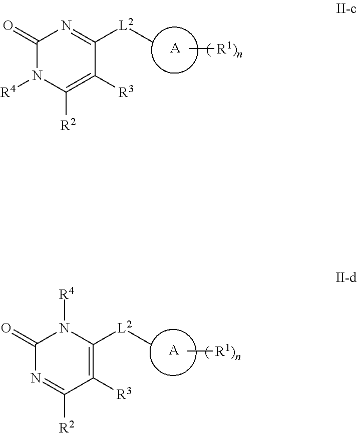

or a pharmaceutically acceptable salt thereof, wherein each of Ring A, R, R¹, R², R³, R⁴, R⁶, R⁷, R⁸, R⁹, R¹⁰, -L²-, X, n, q, and r is as defined above and described in embodiments herein, both singly and in combination.

In some embodiments, the present invention provides a compound of Formula III-b:

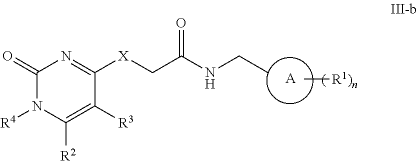

or a pharmaceutically acceptable salt thereof, wherein each of Ring A, R, R¹, R², R³, R⁴, R⁶, R⁷, X, and n is as defined above and described in embodiments herein, both singly and in combination.

In some embodiments, the present invention provides a compound of Formula IV-e or IV-f:

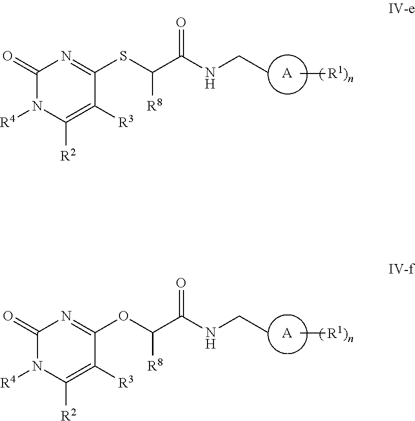

or a pharmaceutically acceptable salt thereof, wherein each of Ring A, R, R¹, R², R³, R⁴, R⁸ and n is as defined above and described in embodiments herein, both singly and in combination.

In some embodiments, the present invention provides a compound of Formula IV-g or IV-h:

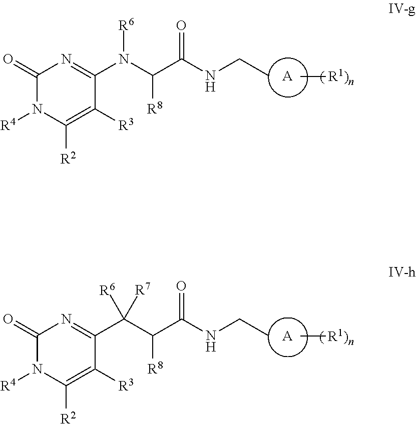

or a pharmaceutically acceptable salt thereof, wherein each of Ring A, R, R¹, R², R³, R⁴, R⁶, R⁷, R⁸, and n is as defined above and described in embodiments herein, both singly and in combination.

In some embodiments, the present invention provides a compound of Formula V-b:

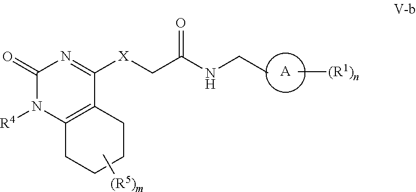

or a pharmaceutically acceptable salt thereof, wherein:
each of Ring A, R, R¹, R⁴, R⁵, R⁶, R⁷, X, m and n is as defined above and described in embodiments herein, both singly and in combination.

In some embodiments, the present invention provides a compound of Formula VI-e or VI-f:

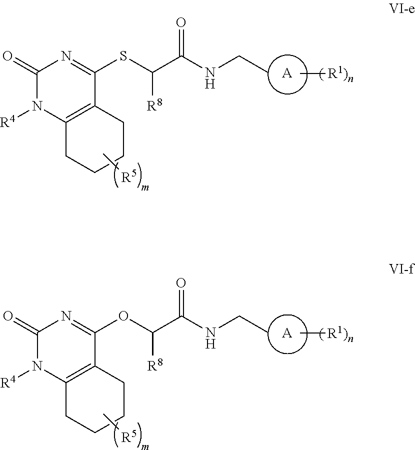

or a pharmaceutically acceptable salt thereof, wherein each of Ring A, R, R¹, R², R³, R⁴, R⁵, R⁸, m and n is as defined above and described in embodiments herein, both singly and in combination.

In some embodiments, the present invention provides a compound of Formula VI-g or VI-h:

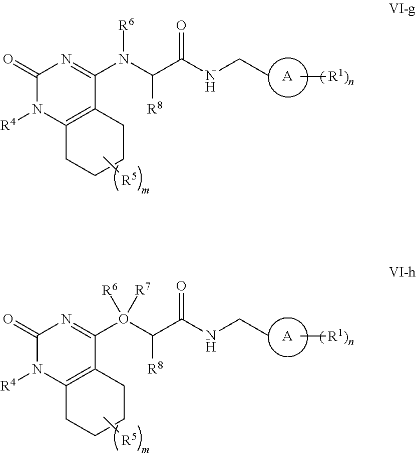

or a pharmaceutically acceptable salt thereof, wherein
each of Ring A, R, R¹, R², R³, R⁴, R⁵, R⁶, R⁷, R⁸, m and n is as defined above and described in embodiments herein, both singly and in combination.

In some embodiments, the present invention provides a compound of Formula VII-b:

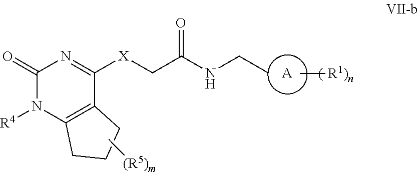

or a pharmaceutically acceptable salt thereof, wherein
each of Ring A, R, R¹, R⁴, R⁵, R⁶, R⁷, X, m and n is as defined above and described in embodiments herein, both singly and in combination.

In some embodiments, the present invention provides a compound of Formula VIII-e or VIII-f:

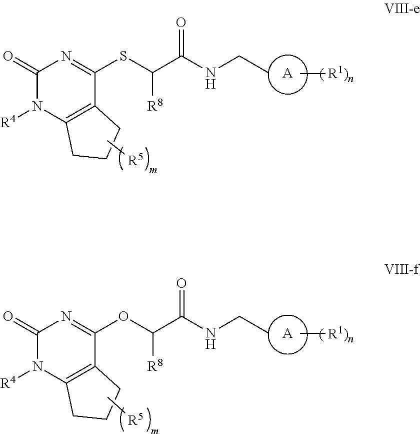

or a pharmaceutically acceptable salt thereof, wherein
each of Ring A, R, R¹, R², R³, R⁴, R⁵, R⁸, m and n is as defined above and described in embodiments herein, both singly and in combination.

In some embodiments, the present invention provides a compound of Formula VIII-g or VIII-h:

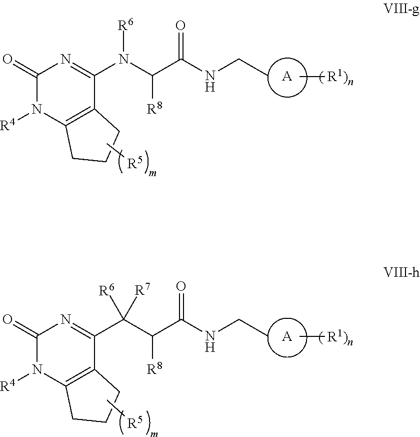

or a pharmaceutically acceptable salt thereof, wherein each of Ring A, R, R¹, R², R³, R⁴, R⁵, R⁶, R⁷, R⁸, m and n is as defined above and described in embodiments herein, both singly and in combination.

In some embodiments, the present invention provides a compound of Formula IX-d, Formula IX-e or Formula IX-f:

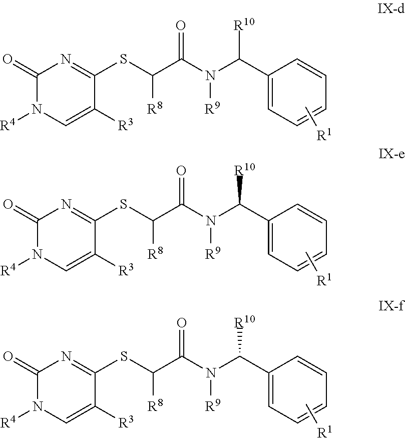

or a pharmaceutically acceptable salt thereof, wherein:
each of R, R¹, R³, R⁴, R⁸, R⁹, and R¹⁰ is as defined above and described in embodiments herein, both singly and in combination.

In some embodiments, R¹ is selected from H, F, Cl, OH, Me, Et, i-Pr,

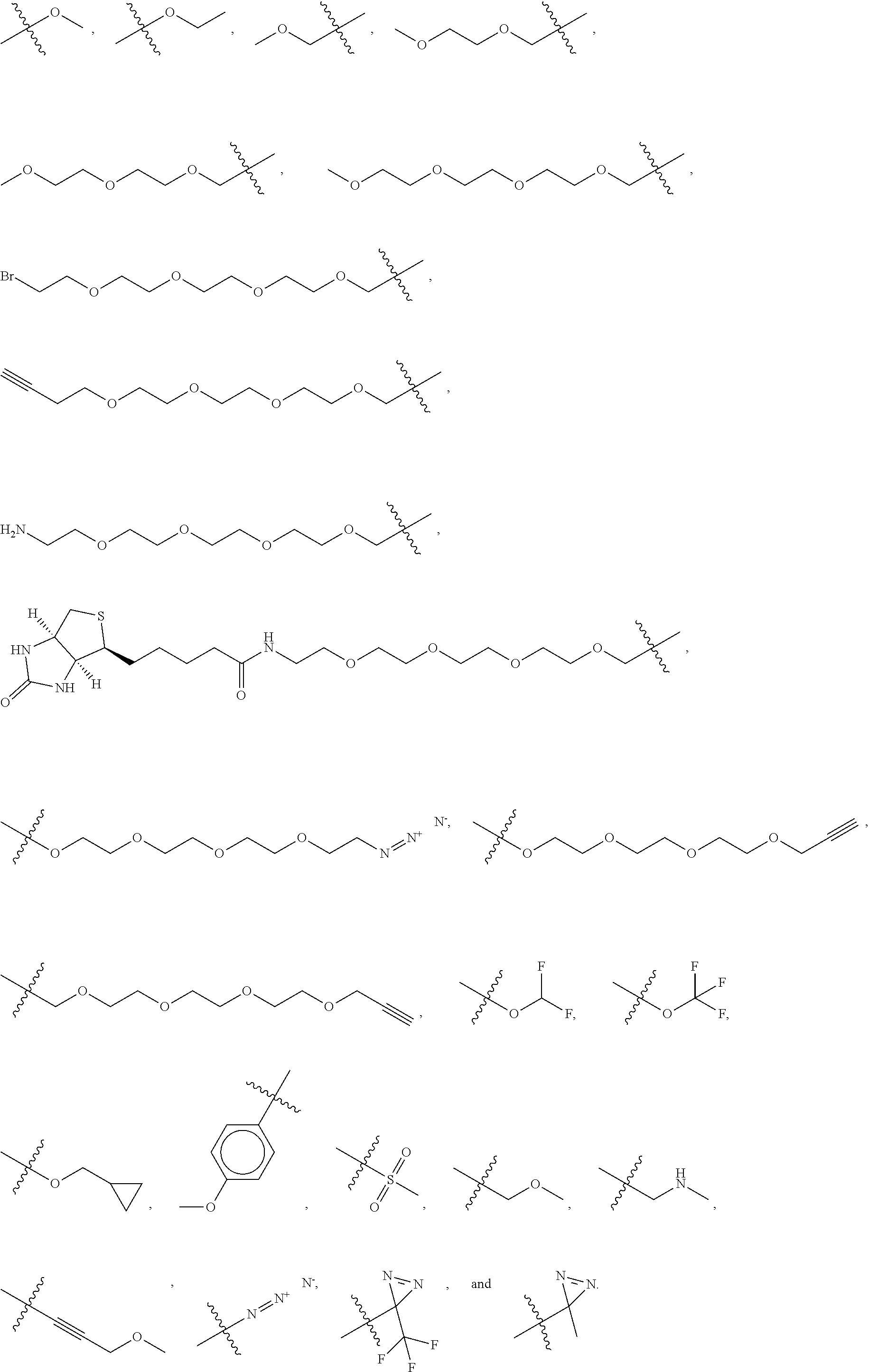

In some embodiments, R³ is selected from H, F, Cl, OH, Me, Et, CF₃, CN, —C(O)NH₂, i-Pr,

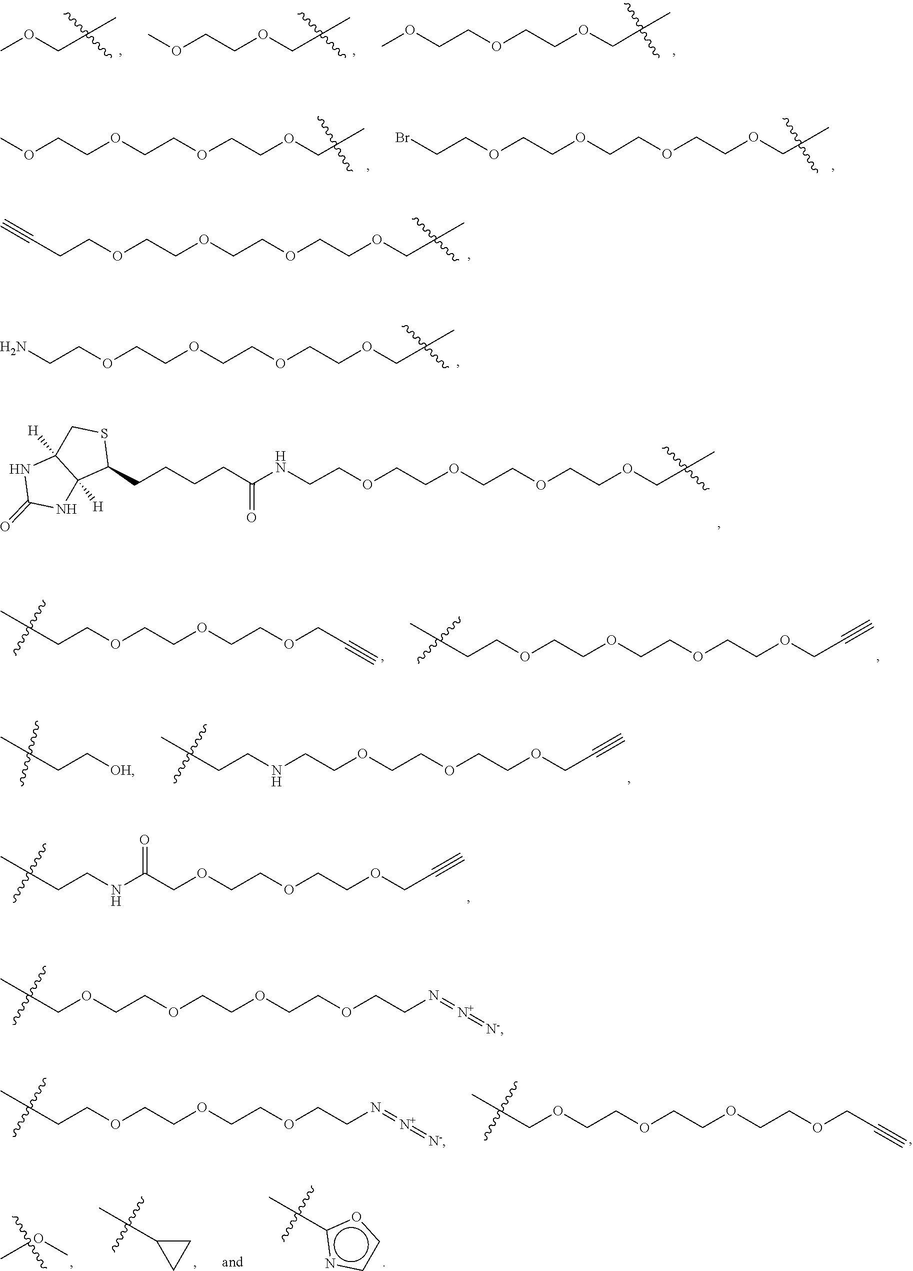

In some embodiments, R⁴ is selected from H, ═O, Me, Et, iPr,

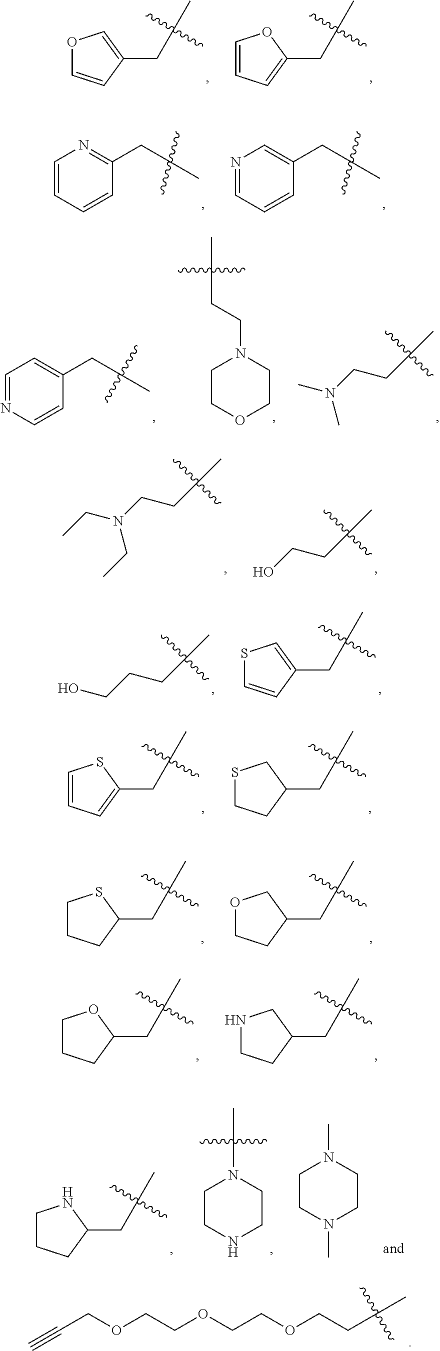

In some embodiments, R⁸ is selected from H, F, Cl, OH, Me, Et, i-Pr,

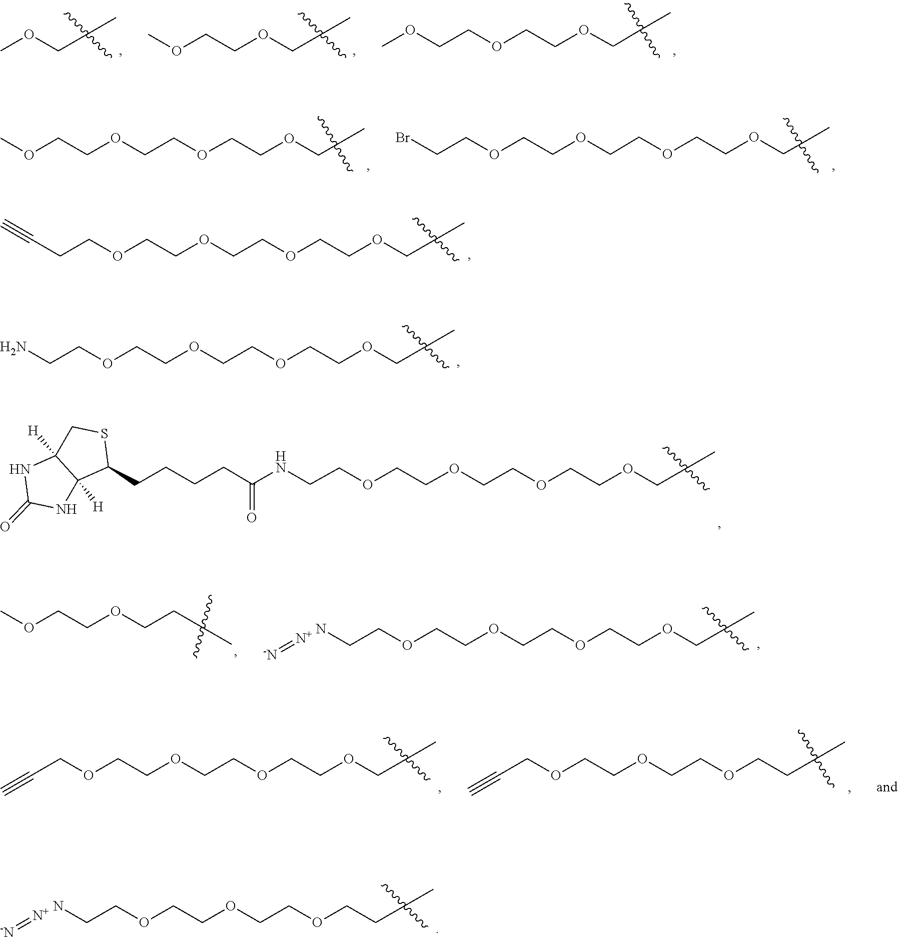

In some embodiments, R⁹ is selected from H, Me, Et, iPr,

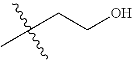 [SMILES: *CCO]

### and OH.

In some embodiments, R¹⁰ is selected from H, Me, Et, iPr,

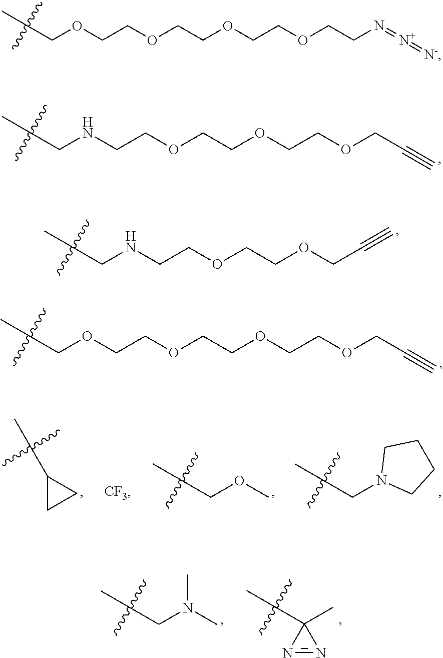

### and CH₃OH.

In some embodiments, the present invention provides a compound of Formula X-d, Formula X-e or Formula X-f:

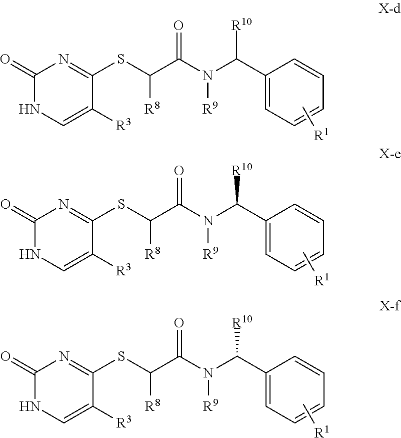

or a pharmaceutically acceptable salt thereof, wherein:
each of R, R¹, R³, R⁸, R¹, R¹⁰, is as defined above and described in embodiments herein, both singly and in combination.

In some embodiments, the present invention provides a compound of Formula XI-d, Formula XI-e or Formula XI-f:

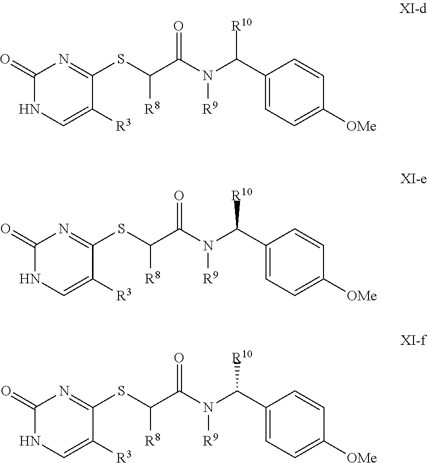

or a pharmaceutically acceptable salt thereof, wherein:
each of R, R³, R⁸, R⁹, R¹⁰, is as defined above and described in embodiments herein, both singly and in combination.

In some embodiments, the present invention provides a compound of Formula XI-d, Formula XII-e or Formula XII-f:

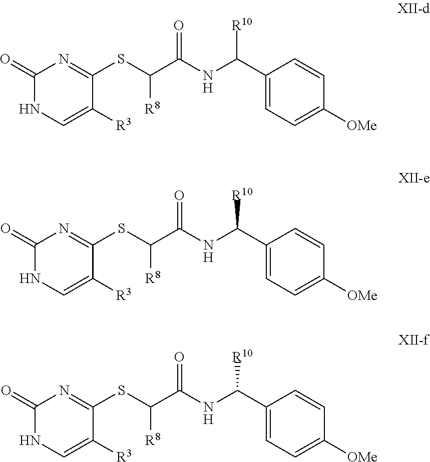

or a pharmaceutically acceptable salt thereof, wherein:
each of R, R³, Ra, R¹⁰, is as defined above and described in embodiments herein, both singly and in combination.

In some embodiments, the present invention provides a compound of Formula XIII-d, Formula XIII-e or Formula XIII-f:

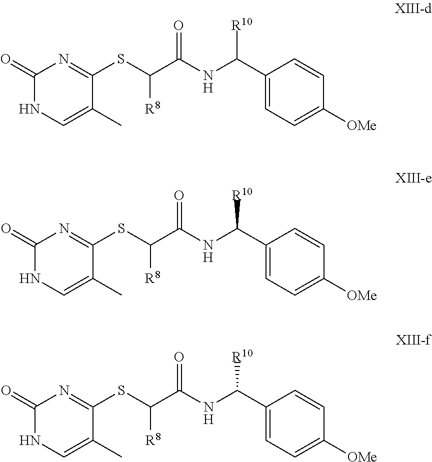

or a pharmaceutically acceptable salt thereof, wherein:
each of R, R¹, R¹⁰, is as defined above and described in embodiments herein, both singly and in combination.

In some embodiments, the present invention provides a compound of Formula XIV-d, Formula XIV-e or Formula XIV-f:

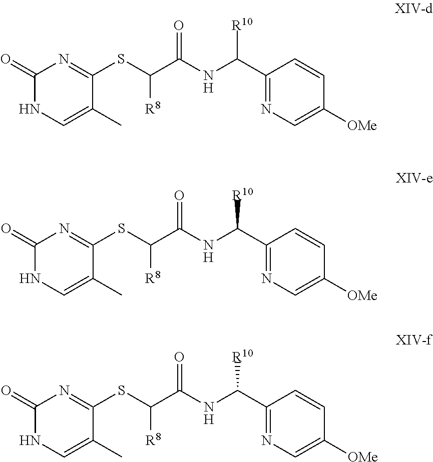

or a pharmaceutically acceptable salt thereof, wherein:
each of R, R⁸, R¹⁰, is as defined above and described in embodiments herein, both singly and in combination.

In some embodiments, the present invention provides a compound of Formula XV-b:

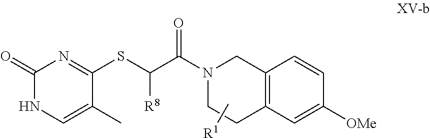

or a pharmaceutically acceptable salt thereof, wherein:
each of R⁸ and R¹ is as defined above and described in embodiments herein, both singly and in combination.

In some embodiments, the present invention provides a compound of Formula XXIII-d, Formula XXIII-e or Formula XXIII-f:

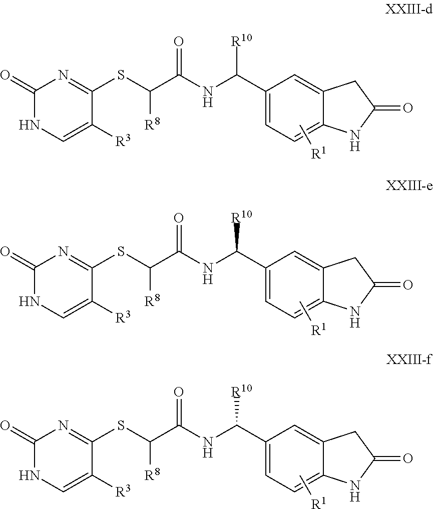

or a pharmaceutically acceptable salt thereof, wherein:
each of R, R¹, R³, R¹⁰, R⁸, -Cy-, is as defined above and described in embodiments herein, both singly and in combination.

In some embodiments, the present invention provides a compound of Formula XXIV-b:

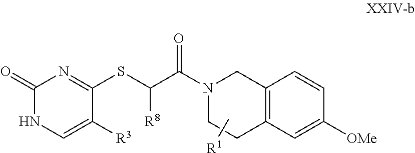

or a pharmaceutically acceptable salt thereof, wherein:
each of R¹, R³ and R⁸ is as defined above and described in embodiments herein, both singly and in combination.

In some embodiments, the present invention provides a compound of Formula XXV-b:

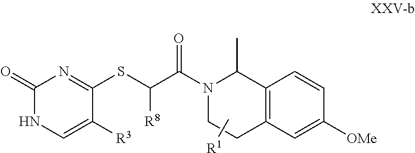

or a pharmaceutically acceptable salt thereof, wherein:
each of R¹, R³ and R⁸ is as defined above and described in embodiments herein, both singly and in combination.

TABLE 1
Exemplary Compounds I-1 to I-163

| | |
|---|---|
| 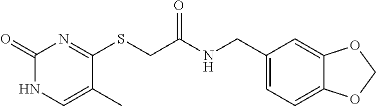 [SMILES: Cc1c[nH]c(=O)nc1SCC(=O)NCc1ccc2c(c1)OCO2] | I-1 |
| 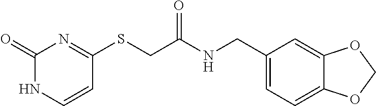 [SMILES: O=C(CSc1cc[nH]c(=O)n1)NCc1ccc2c(c1)OCO2] | I-2 |
| 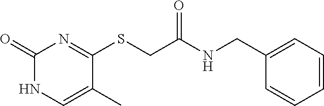 [SMILES: Cc1c[nH]c(=O)nc1SCC(=O)NCc1ccccc1] | I-3 |
| 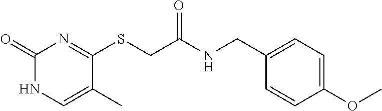 [SMILES: COc1ccc(CNC(=O)CSc2nc(=O)[nH]cc2C)cc1] | I-4 |
| 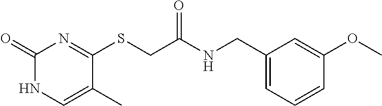 [SMILES: COc1cccc(CNC(=O)CSc2nc(=O)[nH]cc2C)c1] | I-5 |
| 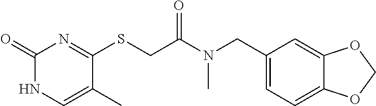 [SMILES: Cc1c[nH]c(=O)nc1SCC(=O)N(C)Cc1ccc2c(c1)OCO2] | I-6 |
| 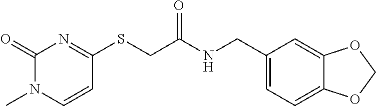 [SMILES: Cn1ccc(SCC(=O)NCc2ccc3c(c2)OCO3)nc1=O] | I-7 |
| 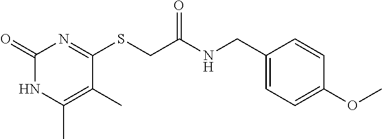 [SMILES: COc1ccc(CNC(=O)CSc2nc(=O)[nH]c(C)c2C)cc1] | I-8 |
| 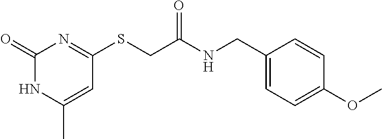 [SMILES: COc1ccc(CNC(=O)CSc2cc(C)[nH]c(=O)n2)cc1] | I-9 |
| 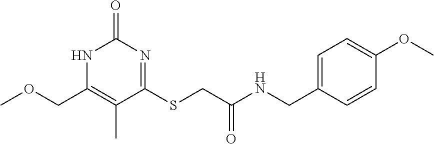 | I-10 |
| 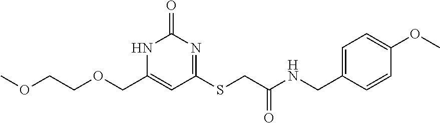 | I-11 |
| 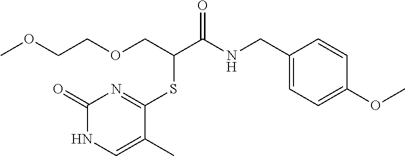 | I-12 |
| 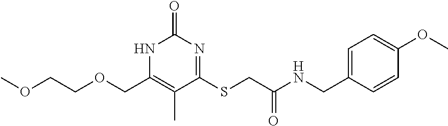 | I-13 |
| 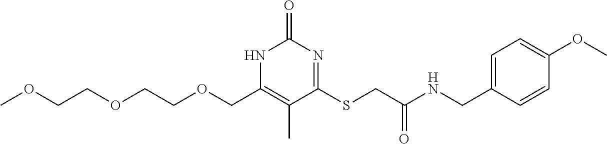 | I-14 |
| 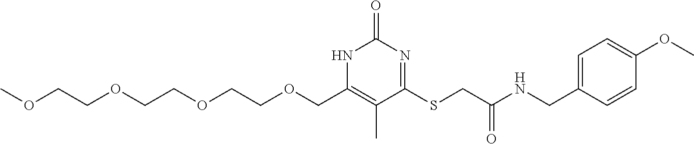 | I-15 |
| 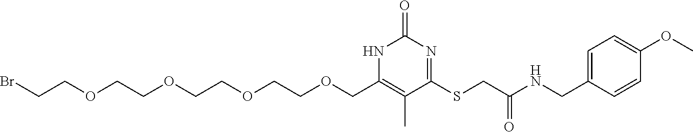 [SMILES: COc1ccc(CNC(=O)CSc2nc(=O)[nH]c(COCCOCCOCCOCCBr)c2C)cc1] | I-16 |
| 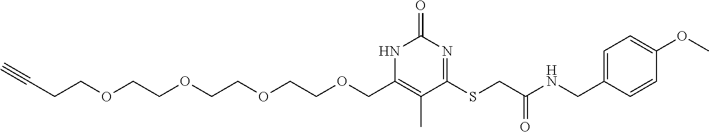 [SMILES: C#CCCOCCOCCOCCOCc1[nH]c(=O)nc(SCC(=O)NCc2ccc(OC)cc2)c1C] | I-17 |
| 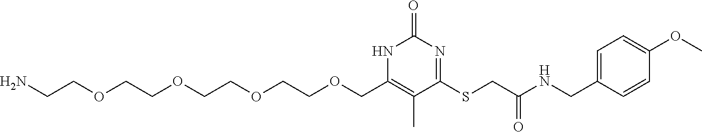 [SMILES: COc1ccc(CNC(=O)CSc2nc(=O)[nH]c(COCCOCCOCCOCCN)c2C)cc1] | I-18 |
| 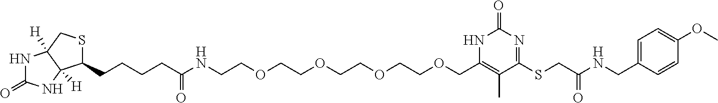 [SMILES: COc1ccc(CNC(=O)CSc2nc(=O)[nH]c(COCCOCCOCCOCCNC(=O)CCCC[C@@H]3SC[C@@H]4NC(=O)N[C@@H]43)c2C)cc1] | I-19 |
| 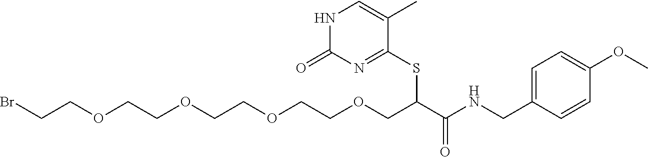 [SMILES: COc1ccc(CNC(=O)C(COCCOCCOCCOCCBr)Sc2nc(=O)[nH]cc2C)cc1] | I-20 |
| 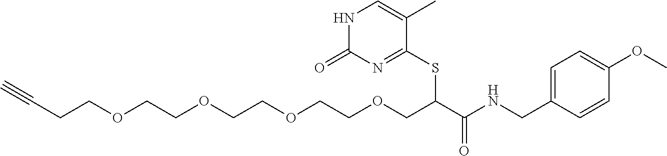 [SMILES: C#CCCOCCOCCOCCOCC(Sc1nc(=O)[nH]cc1C)C(=O)NCc1ccc(OC)cc1] | I-21 |
| 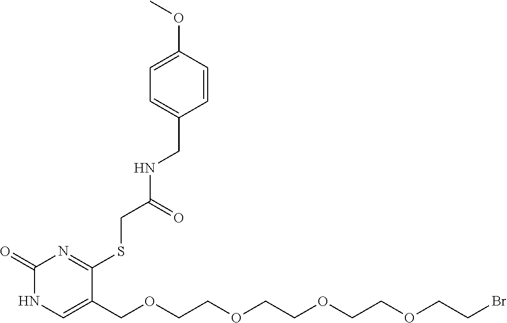 [SMILES: COc1ccc(CNC(=O)CSc2nc(=O)[nH]cc2COCCOCCOCCOCCBr)cc1] | I-22 |
| 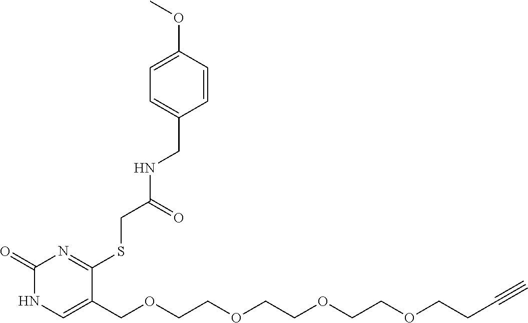 [SMILES: C#CCCOCCOCCOCCOCc1c[nH]c(=O)nc1SCC(=O)NCc1ccc(OC)cc1] | I-23 |
| 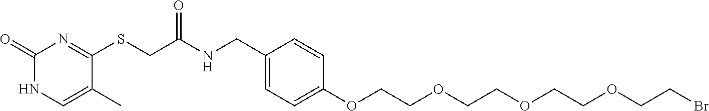 [SMILES: Cc1c[nH]c(=O)nc1SCC(=O)NCc1ccc(OCCOCCOCCOCCBr)cc1] | I-24 |
| 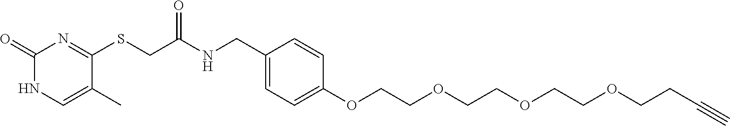 [SMILES: C#CCCOCCOCCOCCOc1ccc(CNC(=O)CSc2nc(=O)[nH]cc2C)cc1] | I-25 |
| 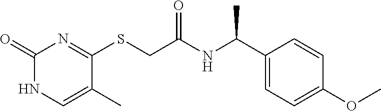 [SMILES: COc1ccc([C@H](C)NC(=O)CSc2nc(=O)[nH]cc2C)cc1] | I-26 |
| 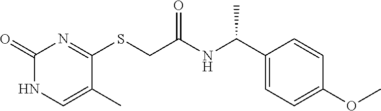 [SMILES: COc1ccc([C@@H](C)NC(=O)CSc2nc(=O)[nH]cc2C)cc1] | I-27 |
| 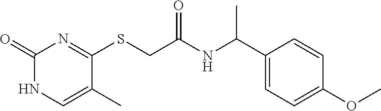 [SMILES: COc1ccc(C(C)NC(=O)CSc2nc(=O)[nH]cc2C)cc1] | I-28 |
| 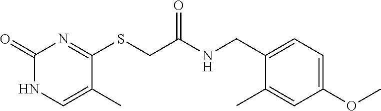 [SMILES: COc1ccc(CNC(=O)CSc2nc(=O)[nH]cc2C)c(C)c1] | I-29 |
| 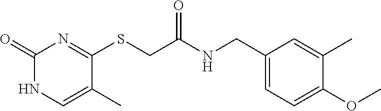 [SMILES: COc1ccc(CNC(=O)CSc2nc(=O)[nH]cc2C)cc1C] | I-30 |
| 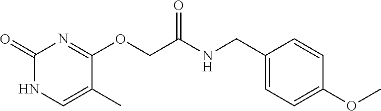 [SMILES: COc1ccc(CNC(=O)COc2nc(=O)[nH]cc2C)cc1] | I-31 |
| 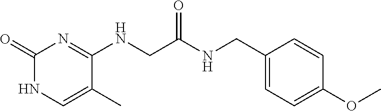 [SMILES: COc1ccc(CNC(=O)CNc2nc(=O)[nH]cc2C)cc1] | I-32 |
| 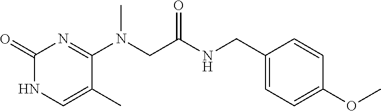 [SMILES: COc1ccc(CNC(=O)CN(C)c2nc(=O)[nH]cc2C)cc1] | I-33 |
| 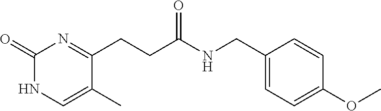 [SMILES: COc1ccc(CNC(=O)CCc2nc(=O)[nH]cc2C)cc1] | I-34 |
| 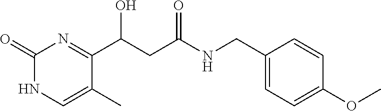 [SMILES: COc1ccc(CNC(=O)CC(O)c2nc(=O)[nH]cc2C)cc1] | I-35 |
| 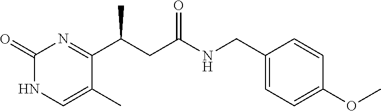 [SMILES: COc1ccc(CNC(=O)C[C@H](C)c2nc(=O)[nH]cc2C)cc1] | I-36 |
| 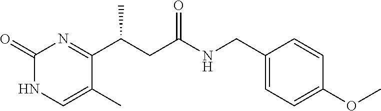 [SMILES: COc1ccc(CNC(=O)C[C@@H](C)c2nc(=O)[nH]cc2C)cc1] | I-37 |
| 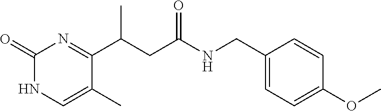 [SMILES: COc1ccc(CNC(=O)CC(C)c2nc(=O)[nH]cc2C)cc1] | I-38 |
| 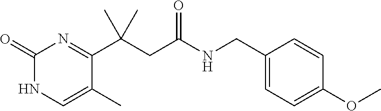 [SMILES: COc1ccc(CNC(=O)CC(C)(C)c2nc(=O)[nH]cc2C)cc1] | I-39 |
| 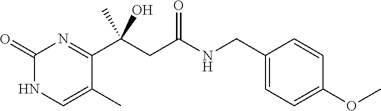 [SMILES: COc1ccc(CNC(=O)C[C@](C)(O)c2nc(=O)[nH]cc2C)cc1] | I-40 |
| 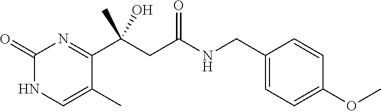 [SMILES: COc1ccc(CNC(=O)C[C@@](C)(O)c2nc(=O)[nH]cc2C)cc1] | I-41 |
| 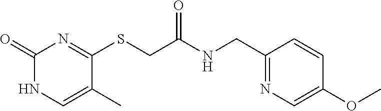 [SMILES: COc1ccc(CNC(=O)CSc2nc(=O)[nH]cc2C)nc1] | I-42 |
| 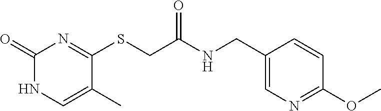 [SMILES: COc1ccc(CNC(=O)CSc2nc(=O)[nH]cc2C)cn1] | I-43 |
| 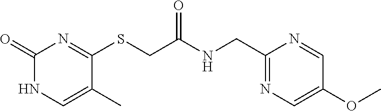 [SMILES: COc1cnc(CNC(=O)CSc2nc(=O)[nH]cc2C)nc1] | I-44 |
| 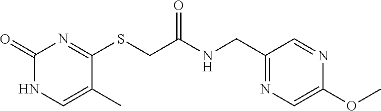 [SMILES: COc1cnc(CNC(=O)CSc2nc(=O)[nH]cc2C)cn1] | I-45 |
| 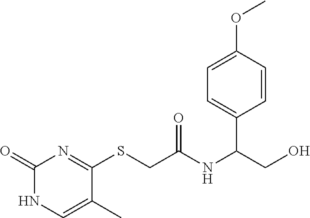 [SMILES: COc1ccc(C(CO)NC(=O)CSc2nc(=O)[nH]cc2C)cc1] | I-46 |
| 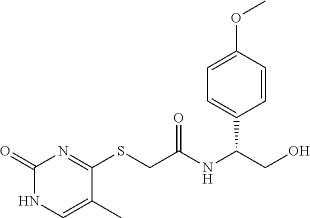 [SMILES: COc1ccc([C@H](CO)NC(=O)CSc2nc(=O)[nH]cc2C)cc1] | I-47 |
| 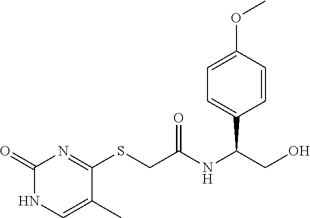 [SMILES: COc1ccc([C@@H](CO)NC(=O)CSc2nc(=O)[nH]cc2C)cc1] | I-48 |
| 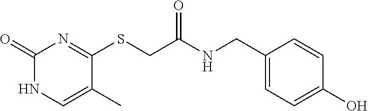 [SMILES: Cc1c[nH]c(=O)nc1SCC(=O)NCc1ccc(O)cc1] | I-49 |
| 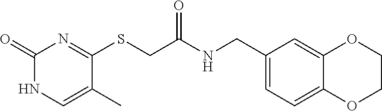 [SMILES: Cc1c[nH]c(=O)nc1SCC(=O)NCc1ccc2c(c1)OCCO2] | I-50 |
| 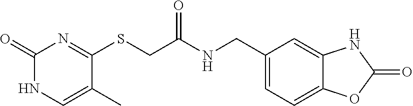 [SMILES: Cc1c[nH]c(=O)nc1SCC(=O)NCc1ccc2oc(=O)[nH]c2c1] | I-51 |
| 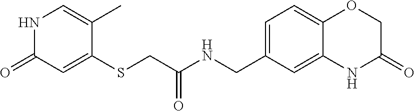 [SMILES: Cc1c[nH]c(=O)cc1SCC(=O)NCc1ccc2c(c1)NC(=O)CO2] | I-52 |
| 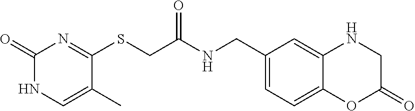 [SMILES: Cc1c[nH]c(=O)nc1SCC(=O)NCc1ccc2c(c1)NCC(=O)O2] | I-53 |
| 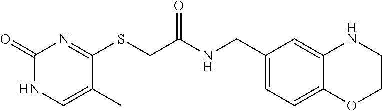 [SMILES: Cc1c[nH]c(=O)nc1SCC(=O)NCc1ccc2c(c1)NCCO2] | I-54 |
| 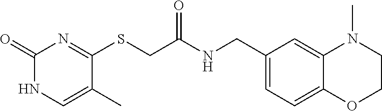 [SMILES: Cc1c[nH]c(=O)nc1SCC(=O)NCc1ccc2c(c1)N(C)CCO2] | I-55 |
| 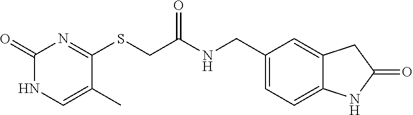 [SMILES: Cc1c[nH]c(=O)nc1SCC(=O)NCc1ccc2c(c1)CC(=O)N2] | I-56 |
| 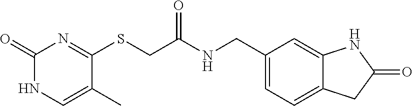 [SMILES: Cc1c[nH]c(=O)nc1SCC(=O)NCc1ccc2c(c1)NC(=O)C2] | I-57 |
| 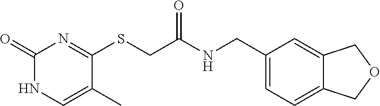 [SMILES: Cc1c[nH]c(=O)nc1SCC(=O)NCc1ccc2c(c1)COC2] | I-58 |
| 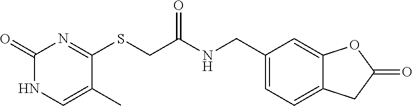 [SMILES: Cc1c[nH]c(=O)nc1SCC(=O)NCc1ccc2c(c1)OC(=O)C2] | I-59 |
| 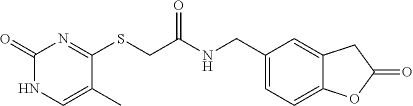 [SMILES: Cc1c[nH]c(=O)nc1SCC(=O)NCc1ccc2c(c1)CC(=O)O2] | I-60 |
| 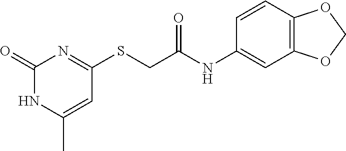 [SMILES: Cc1cc(SCC(=O)Nc2ccc3c(c2)OCO3)nc(=O)[nH]1] | I-61 |
| 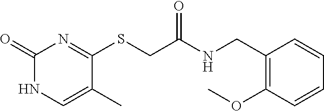 [SMILES: COc1ccccc1CNC(=O)CSc1nc(=O)[nH]cc1C] | I-62 |
| 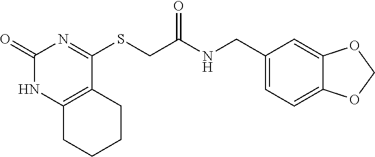 [SMILES: O=C(CSc1nc(=O)[nH]c2c1CCCC2)NCc1ccc2c(c1)OCO2] | I-63 |
| 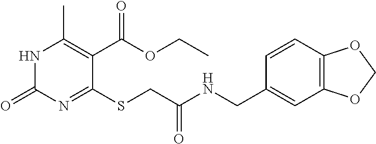 [SMILES: CCOC(=O)c1c(SCC(=O)NCc2ccc3c(c2)OCO3)nc(=O)[nH]c1C] | I-64 |
| 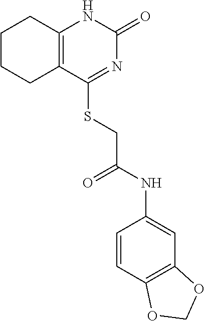 [SMILES: O=C(CSc1nc(=O)[nH]c2c1CCCC2)Nc1ccc2c(c1)OCO2] | I-65 |
| 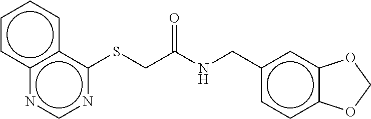 | I-66 |
| 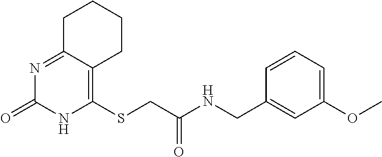 [SMILES: COc1cccc(CNC(=O)CSc2[nH]c(=O)nc3c2CCCC3)c1] | I-67 |
| 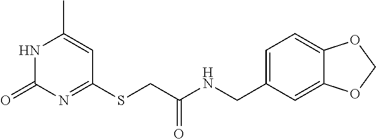 [SMILES: Cc1cc(SCC(=O)NCc2ccc3c(c2)OCO3)nc(=O)[nH]1] | I-68 |
| 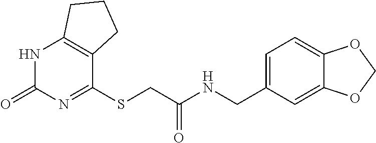 [SMILES: O=C(CSc1nc(=O)[nH]c2c1CCC2)NCc1ccc2c(c1)OCO2] | I-69 |
| 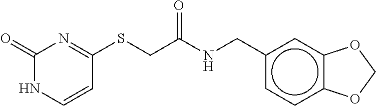 | I-70 |
| 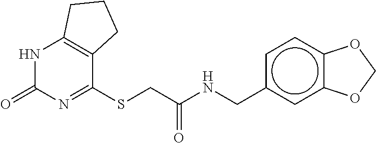 | I-71 |
| 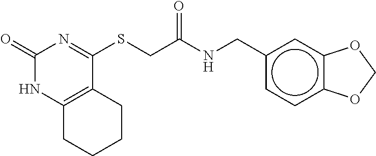 | I-72 |
| 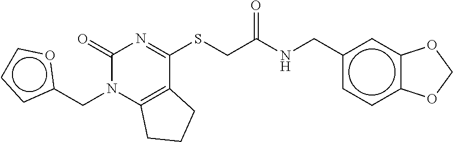 | I-73 |
| 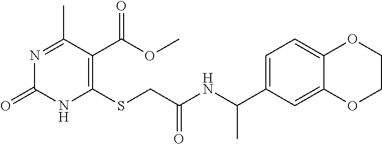 [SMILES: COC(=O)c1c(C)nc(=O)[nH]c1SCC(=O)NC(C)c1ccc2c(c1)OCCO2] | I-74 |
| 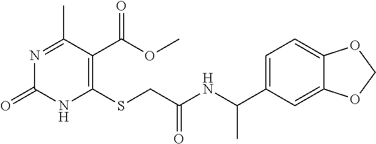 [SMILES: COC(=O)c1c(C)nc(=O)[nH]c1SCC(=O)NC(C)c1ccc2c(c1)OCO2] | I-75 |
| 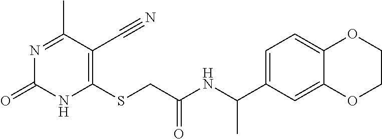 [SMILES: Cc1nc(=O)[nH]c(SCC(=O)NC(C)c2ccc3c(c2)OCCO3)c1C#N] | I-76 |
| 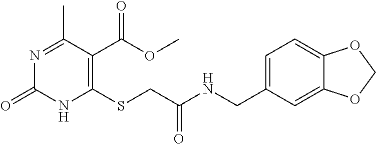 [SMILES: COC(=O)c1c(C)nc(=O)[nH]c1SCC(=O)NCc1ccc2c(c1)OCO2] | I-77 |
| 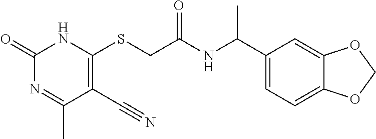 [SMILES: Cc1nc(=O)[nH]c(SCC(=O)NC(C)c2ccc3c(c2)OCO3)c1C#N] | I-78 |
| 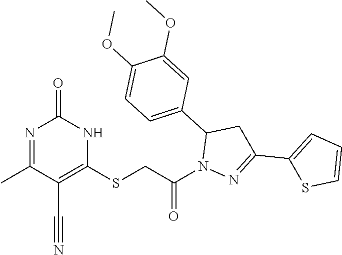 [SMILES: COc1ccc(C2CC(c3cccs3)=NN2C(=O)CSc2[nH]c(=O)nc(C)c2C#N)cc1OC] | I-79 |
| 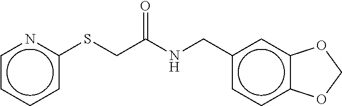 | I-80 |
| 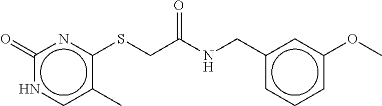 | I-81 |
| 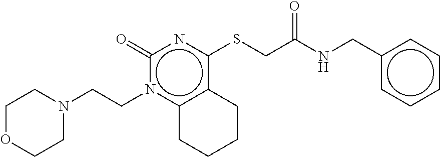 | I-82 |
| 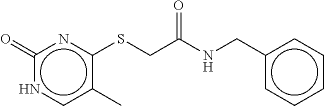 | I-83 |
| 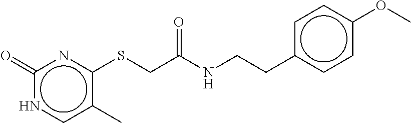 | I-84 |
| 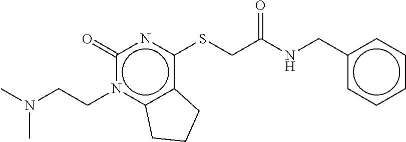 | I-85 |
| 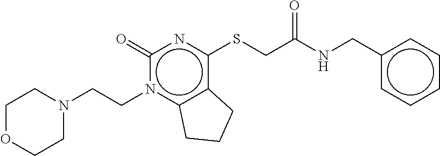 | I-86 |
| 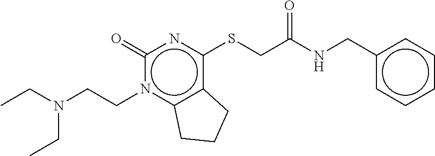 | I-87 |
| 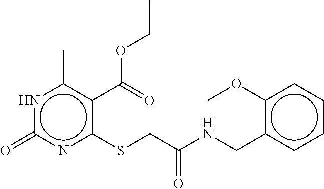 | I-88 |
| 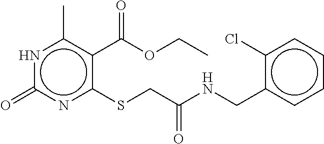 | I-89 |
| 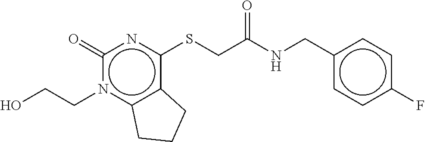 | I-90 |
| 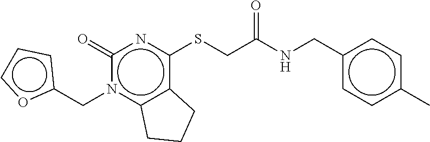 | I-91 |
| 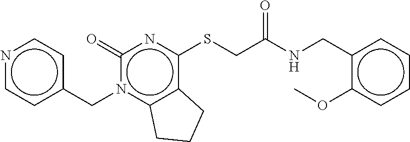 | I-92 |
| 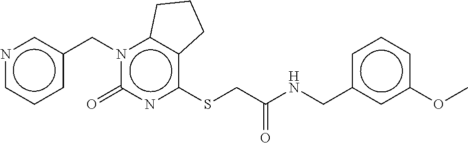 | I-93 |
| 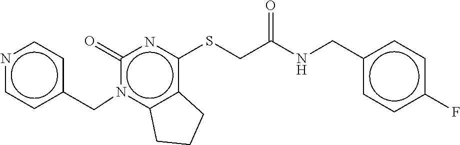 | I-94 |
| 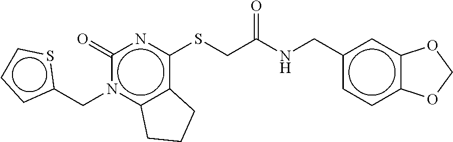 | I-95 |
| 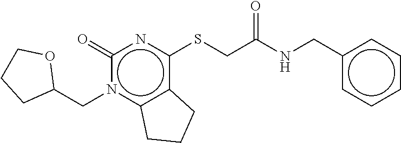 | I-96 |
| 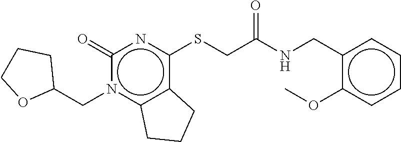 | I-97 |
| 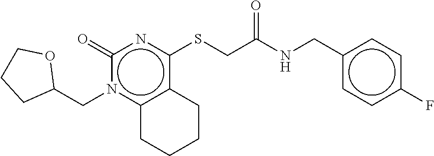 | I-98 |
| 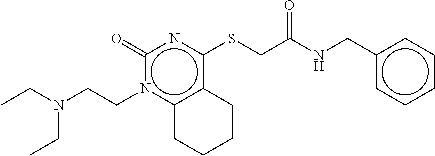 | I-99 |
| 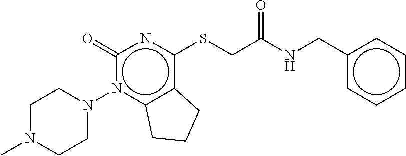 | I-100 |
| 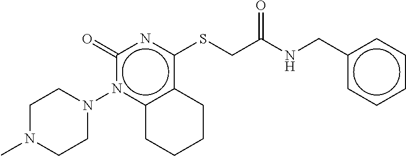 | I-101 |
| 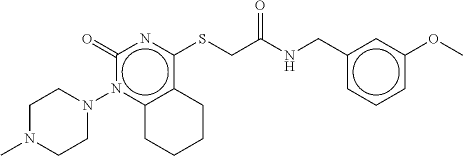 | I-102 |
| 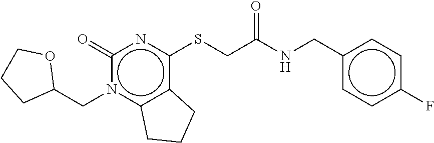 | I-103 |
| 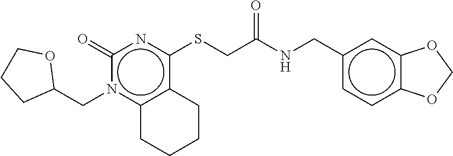 | I-104 |
| 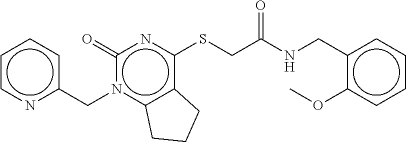 | I-105 |
| 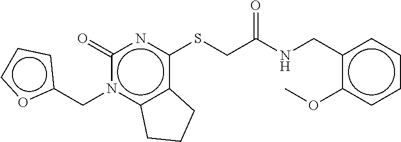 | I-106 |
| 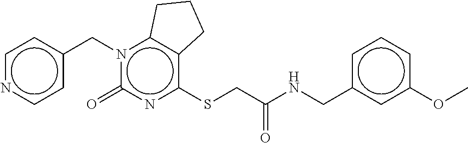 | I-107 |
| 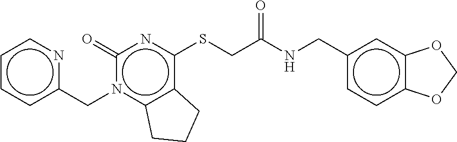 | I-108 |
| 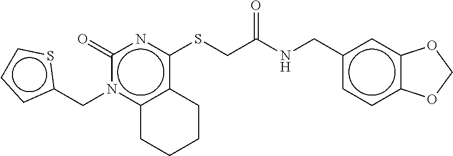 | I-109 |
| 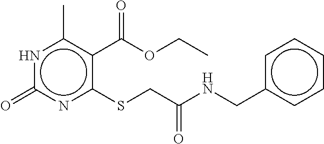 | I-110 |
| 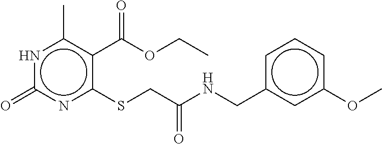 | I-111 |
| 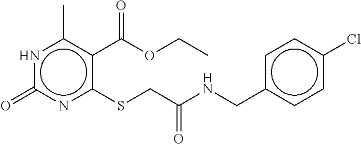 | I-112 |
| 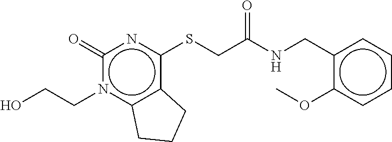 | I-113 |
| 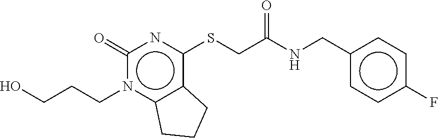 | I-114 |
| 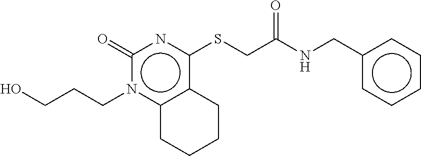 | I-115 |
| 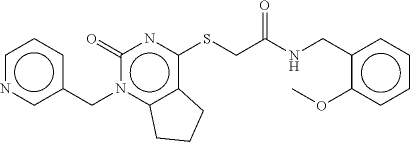 | I-116 |
| 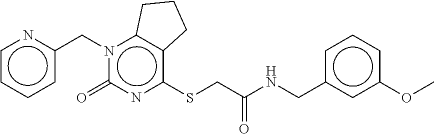 | I-117 |
| 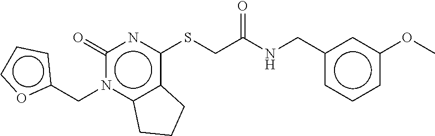 | I-118 |
| 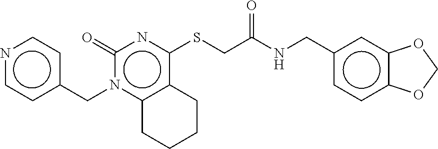 | I-119 |
| 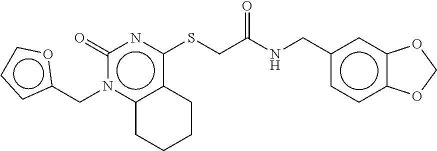 | I-120 |
| 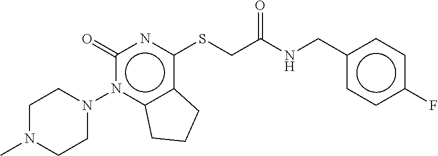 | I-121 |
| 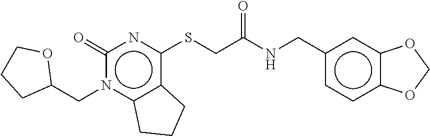 | I-122 |
| 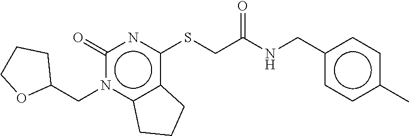 | I-123 |
| 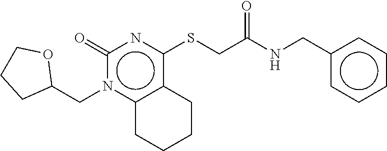 | I-124 |
| 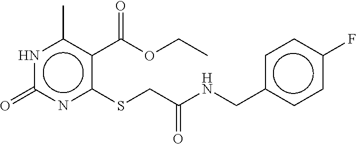 | I-125 |
| 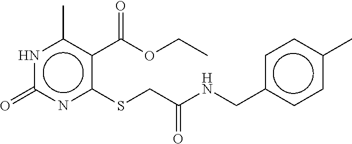 | I-126 |
| 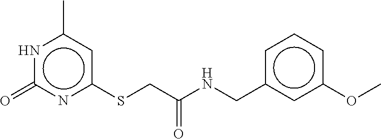 | I-127 |
| 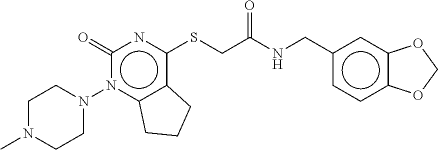 | I-128 |
| 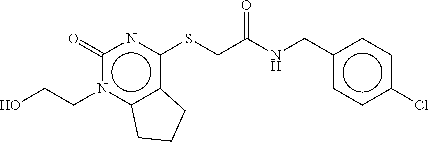 | I-129 |
| 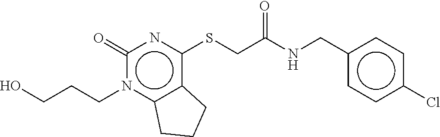 | I-130 |
| 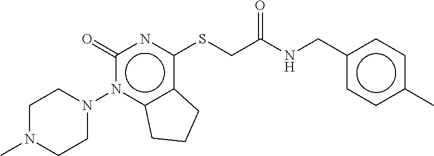 | I-131 |
| 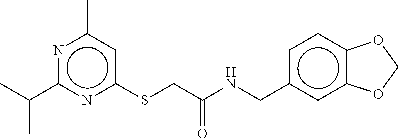 | I-132 |
| 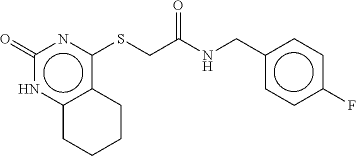 | I-133 |
| 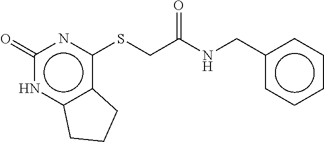 | I-134 |
| 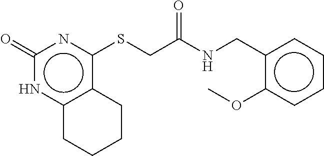 | I-135 |
| 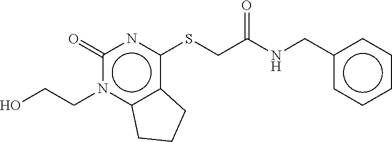 | I-136 |
| 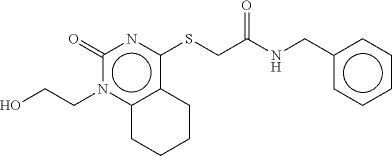 | I-137 |
| 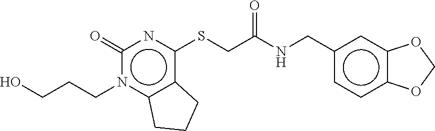 | I-138 |
| 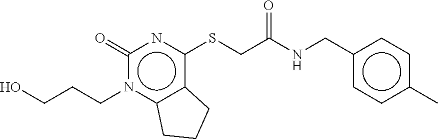 | I-139 |
| 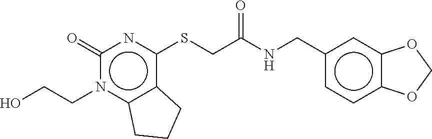 | I-140 |
| 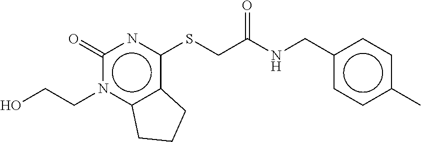 | I-141 |
| 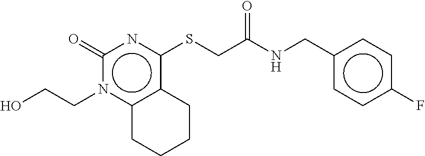 | I-142 |
| 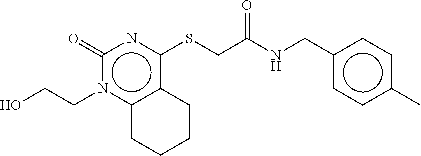 | I-143 |
| 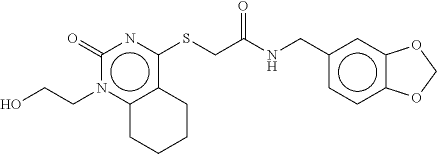 | I-144 |
| 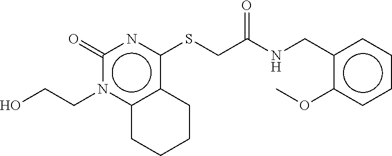 | I-145 |
| 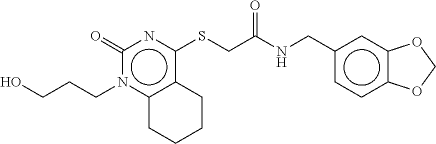 | I-146 |
| 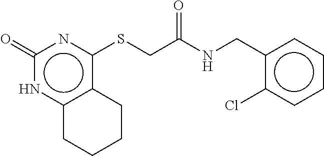 | I-147 |
| 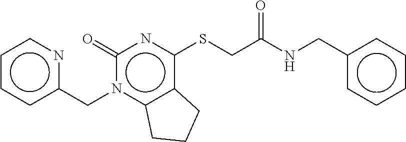 | I-149 |
| 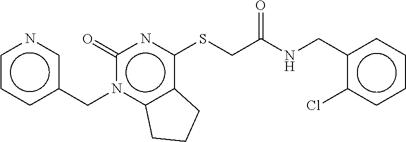 | I-149 |
| 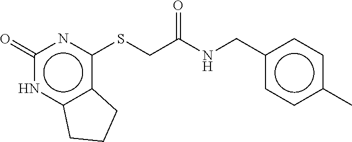 | I-150 |
| 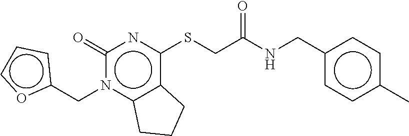 | I-151 |
| 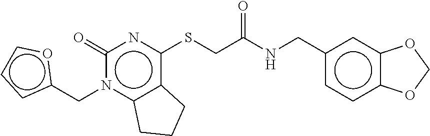 | I-152 |
| 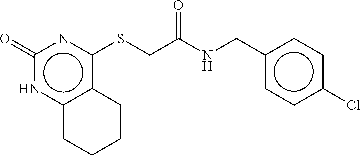 | I-153 |
| 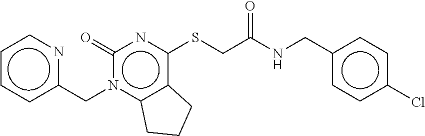 | I-154 |
| 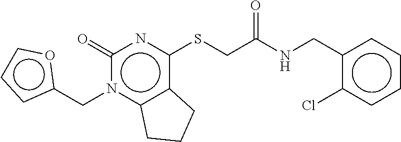 | I-155 |
| 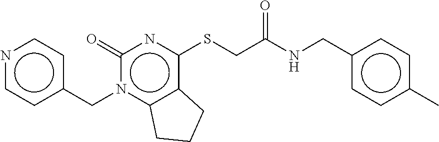 | I-156 |
| 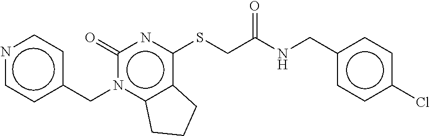 | I-157 |
| 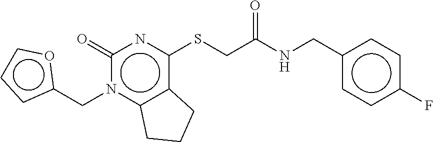 | I-158 |
| 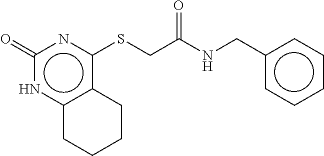 | I-159 |
| 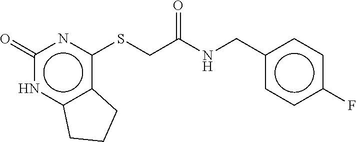 | I-160 |
| 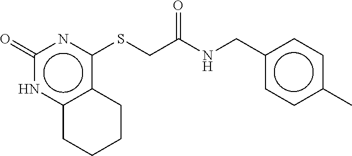 | I-161 |
| 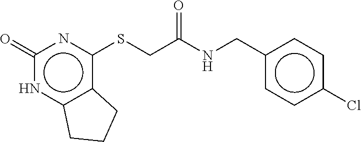 | I-162 |
| 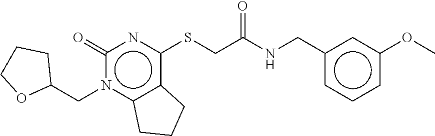 | I-163 |

TABLE 1A
Exemplary Compounds I-164 to I-321

| | |
|---|---|
| 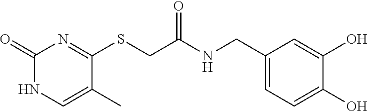 [SMILES: Cc1c[nH]c(=O)nc1SCC(=O)NCc1ccc(O)c(O)c1] | I-164 |
| 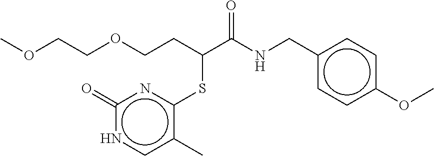 | I-165 |
| 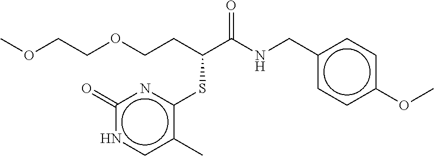 | I-166 |
| 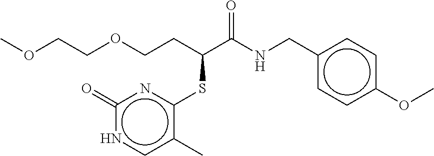 | I-167 |
| 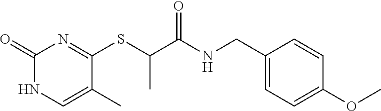 [SMILES: COc1ccc(CNC(=O)C(C)Sc2nc(=O)[nH]cc2C)cc1] | I-168 |
| 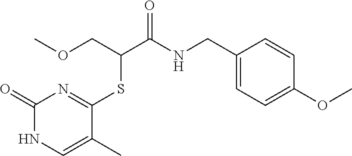 [SMILES: COCC(Sc1nc(=O)[nH]cc1C)C(=O)NCc1ccc(OC)cc1] | I-169 |
| 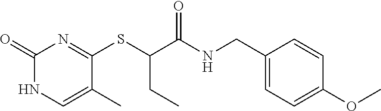 [SMILES: CCC(Sc1nc(=O)[nH]cc1C)C(=O)NCc1ccc(OC)cc1] | I-170 |
| 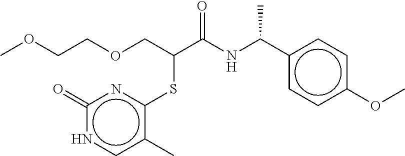 | I-171 |
| 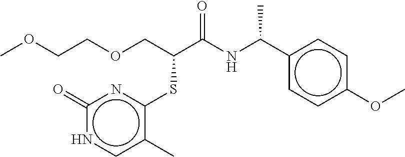 | I-172 |
| 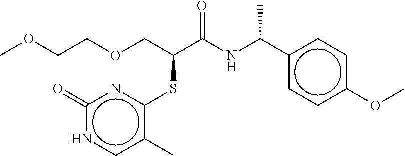 | I-173 |
| 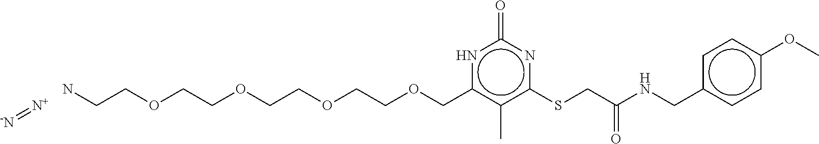 | I-174 |
| 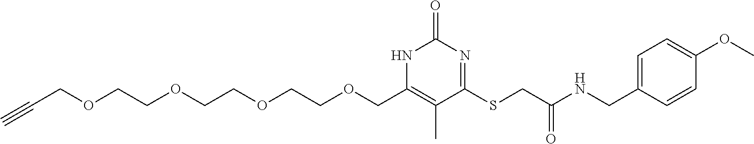 [SMILES: C#CCOCCOCCOCCOCc1[nH]c(=O)nc(SCC(=O)NCc2ccc(OC)cc2)c1C] | I-175 |
| 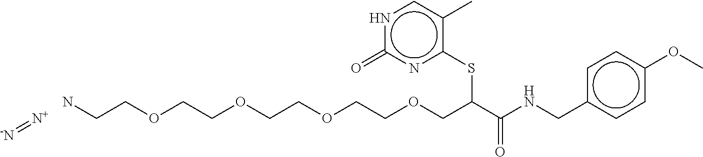 | I-176 |
| 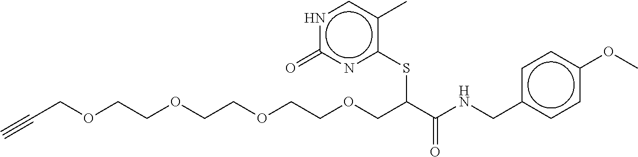 | I-177 |
| 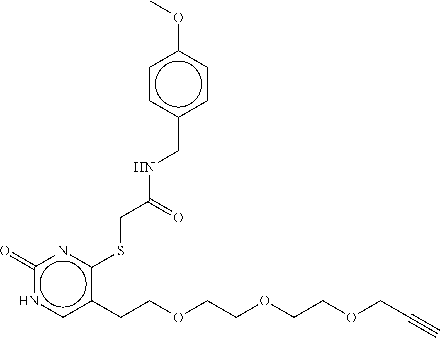 | I-178 |
| 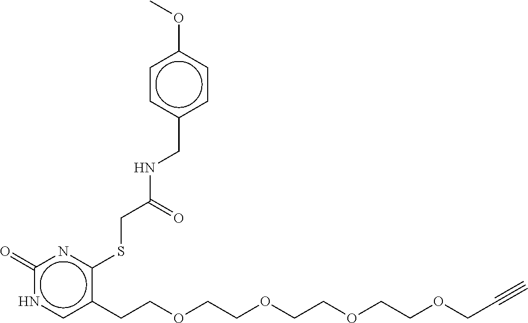 | I-179 |
| 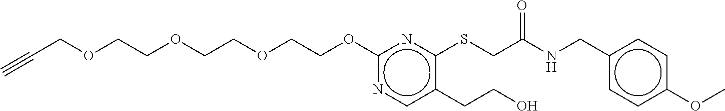 | I-180 |
| 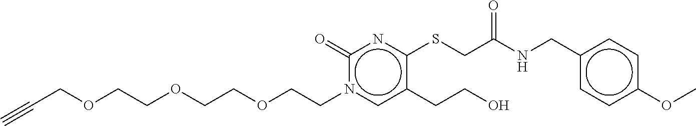 | I-181 |
| 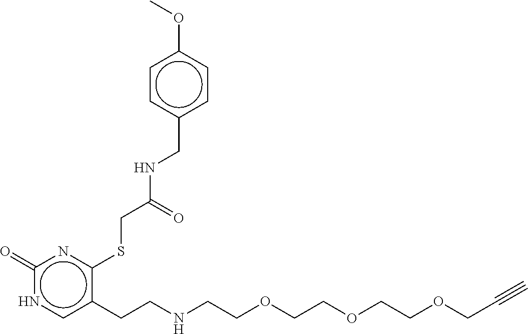 | I-182 |
| 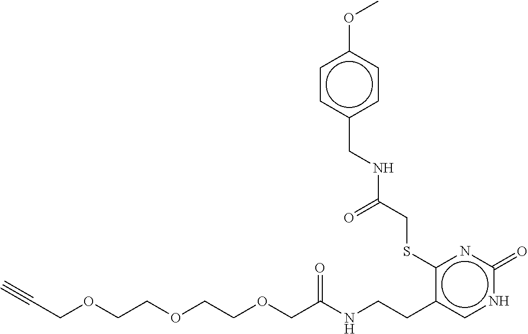 | I-183 |
| 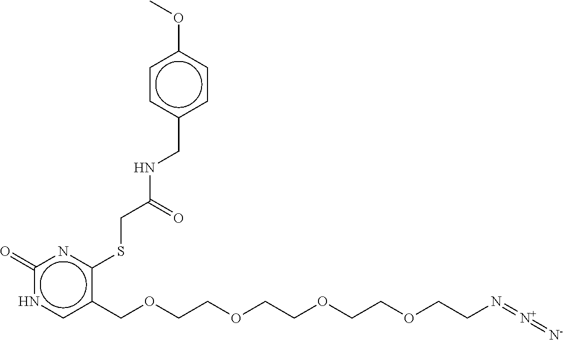 | I-184 |
| 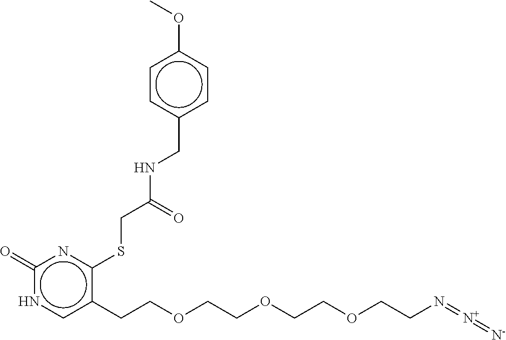 | I-185 |
| 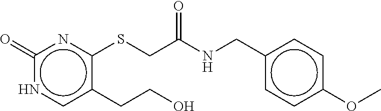 | I-186 |
| 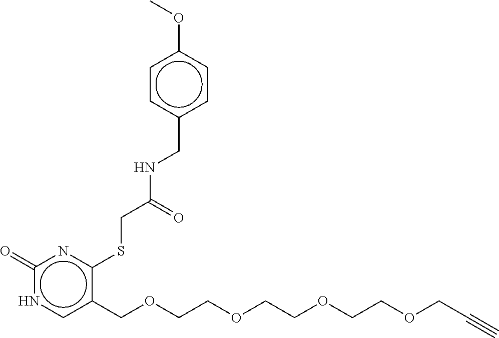 | I-187 |
| 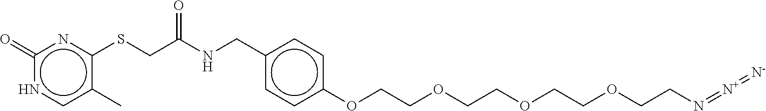 | I-188 |
| 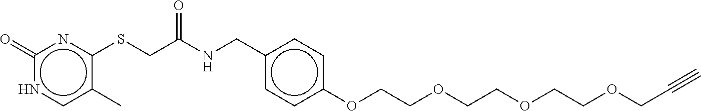 | I-189 |
| 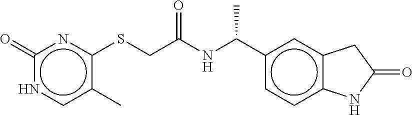 | I-190 |
| 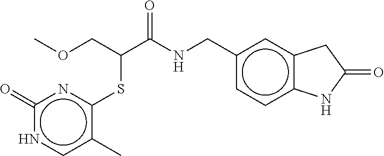 | I-191 |
| 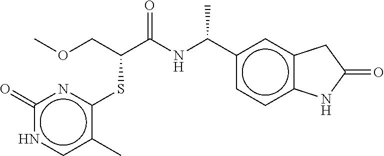 | I-192 |
| 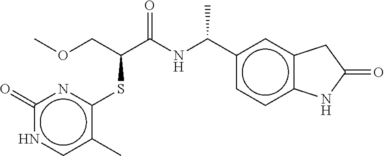 | I-193 |
| 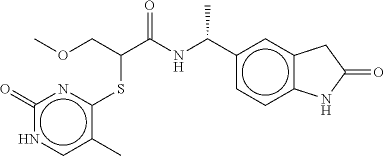 | I-194 |
| 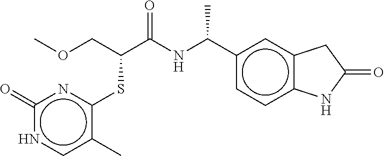 | I-195 |
| 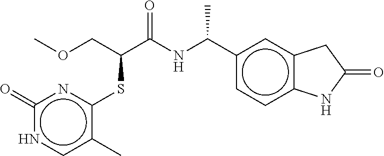 | I-196 |
| 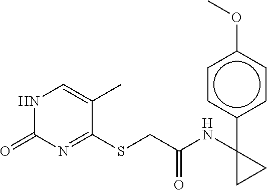 | I-197 |
| 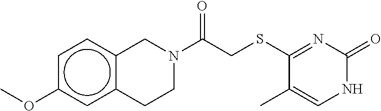 | I-198 |
| 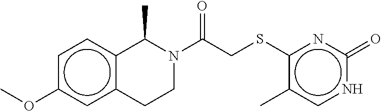 | I-199 |
| 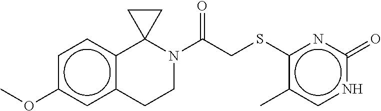 | I-200 |
| 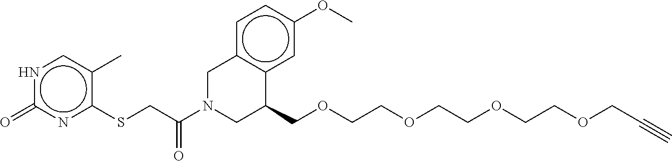 | I-201 |
| 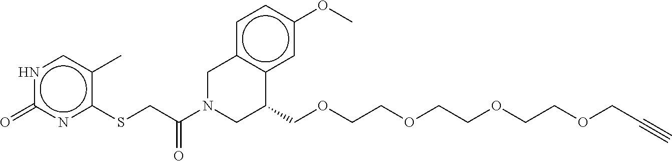 | I-202 |
| 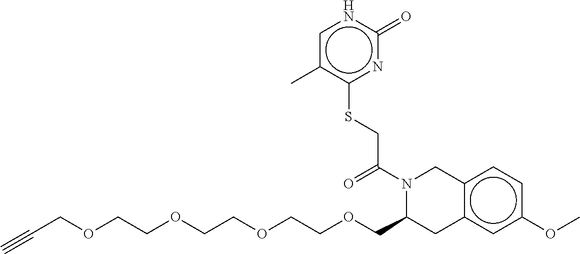 | I-203 |
| 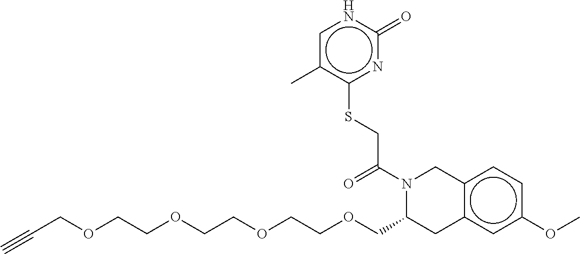 | I-204 |
| 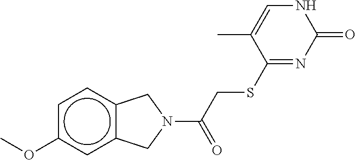 | I-205 |
| 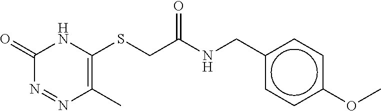 | I-206 |
| 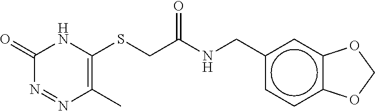 | I-207 |
| 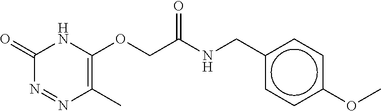 | I-208 |
| 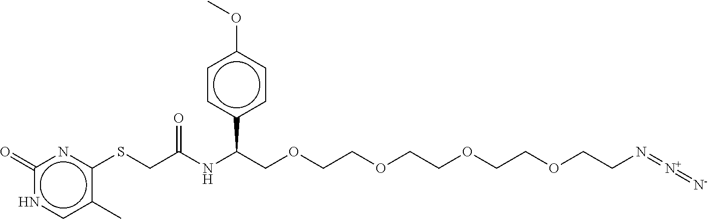 | I-209 |
| 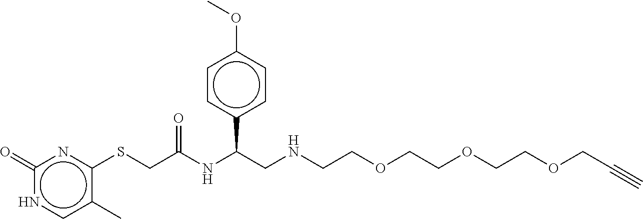 | I-210 |
| 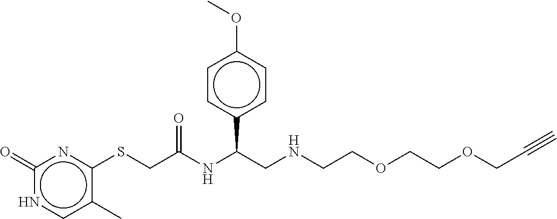 | I-211 |
| 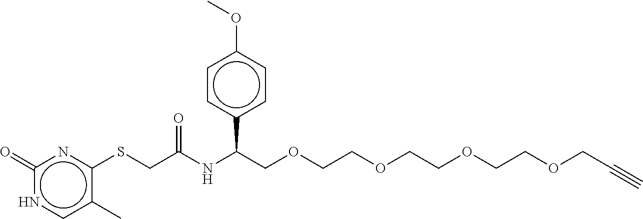 | I-212 |
| 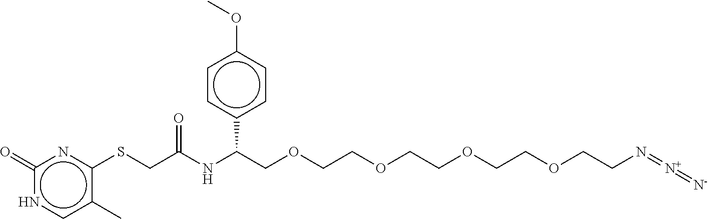 | I-213 |
| 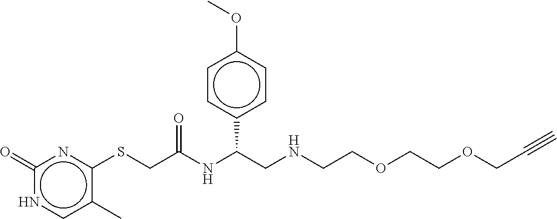 | I-214 |
| 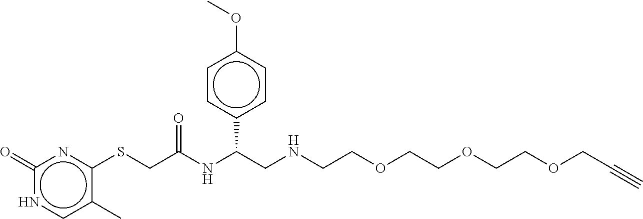 | I-215 |
| 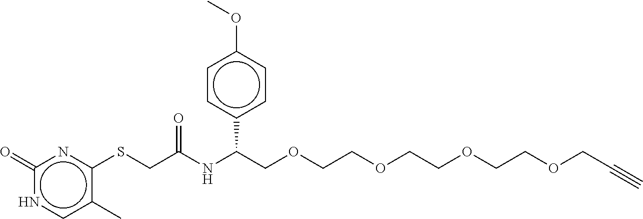 | I-216 |
| 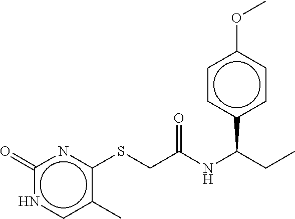 | I-217 |
| 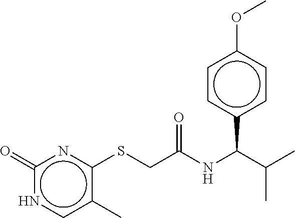 | I-218 |
| 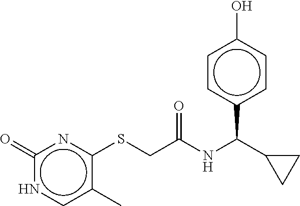 | I-219 |
| 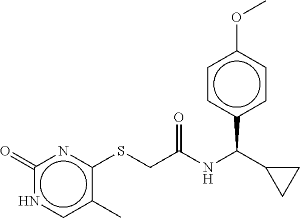 | I-220 |
| 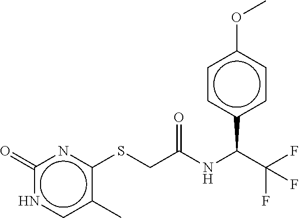 | I-221 |
| 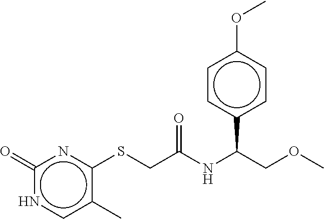 | I-222 |
| 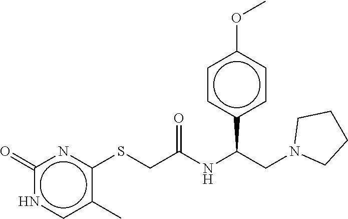 | I-223 |
| 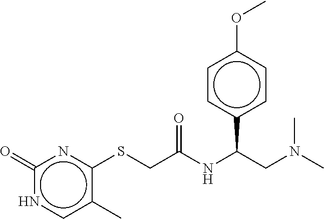 | I-224 |
| 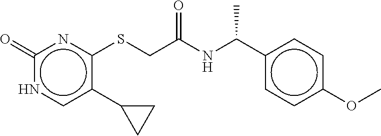 | I-225 |
| 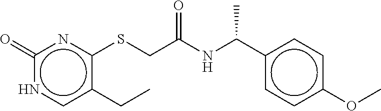 | I-226 |
| 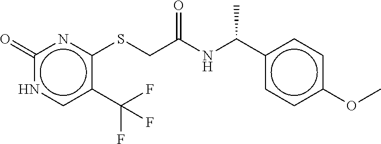 | I-227 |
| 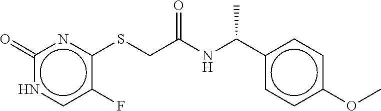 | I-228 |
| 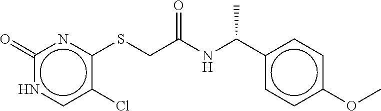 | I-229 |
| 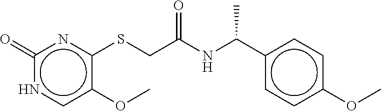 | I-230 |
| 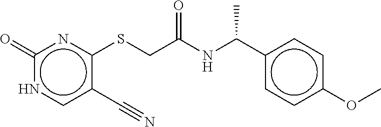 | I-231 |
| 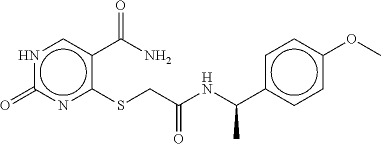 | I-232 |
| 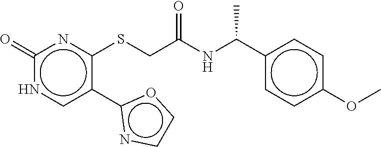 | I-233 |
| 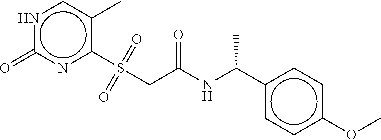 | I-234 |
| 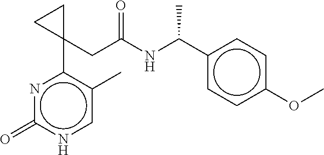 | I-235 |
| 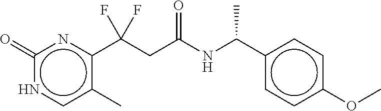 | I-236 |
| 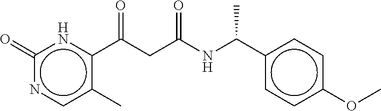 | I-237 |
| 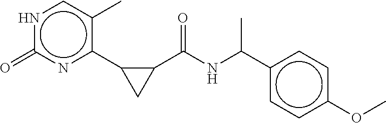 | I-238 |
| 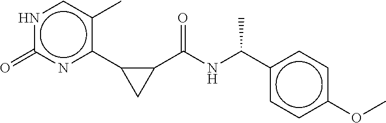 | I-239 |
| 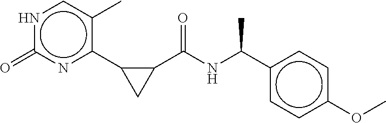 | I-240 |
| 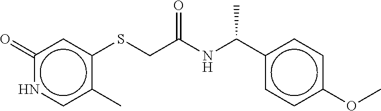 | I-241 |
| 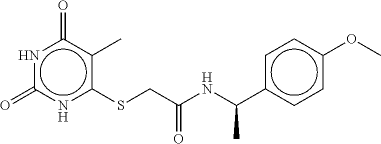 | I-242 |
| 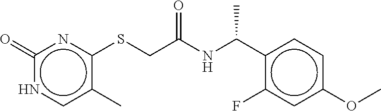 | I-243 |
| 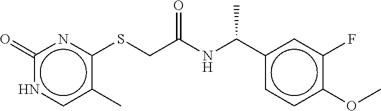 | I-244 |
| 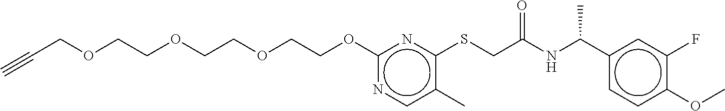 | I-245 |
| 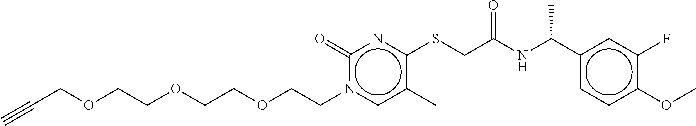 | I-246 |
| 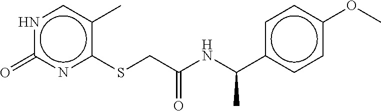 | I-247 |
| 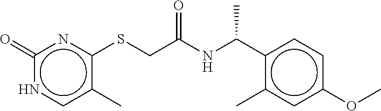 | I-248 |
| 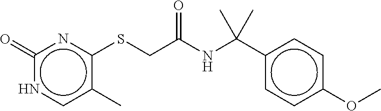 | I-249 |
| 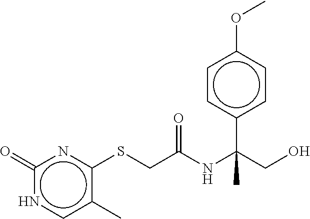 | I-250 |
| 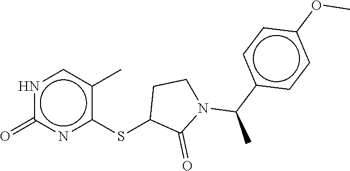 | I-251 |
| 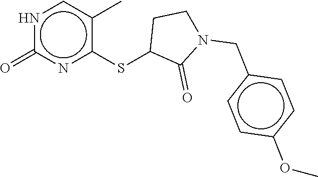 | I-252 |
| 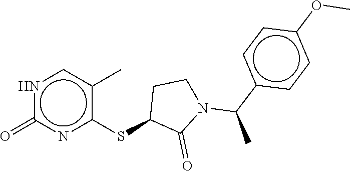 | I-253 |
| 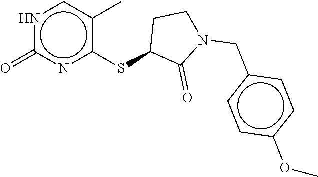 | I-254 |
| 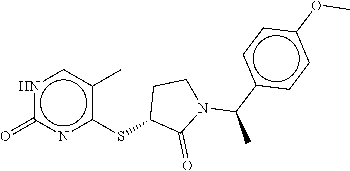 | I-255 |
| 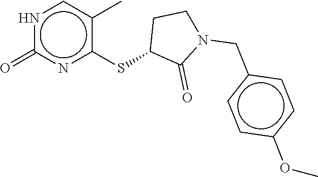 | I-256 |
| 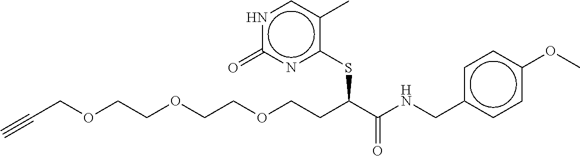 | I-257 |
| 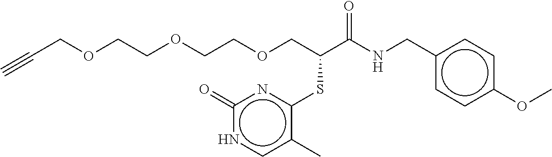 | I-258 |
| 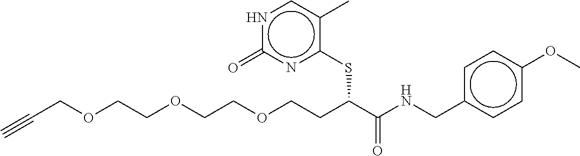 | I-259 |
| 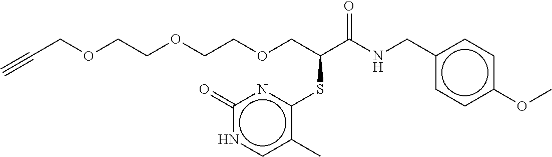 | I-260 |
| 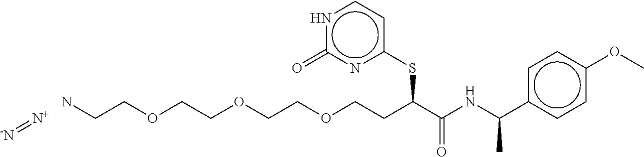 | I-261 |
| 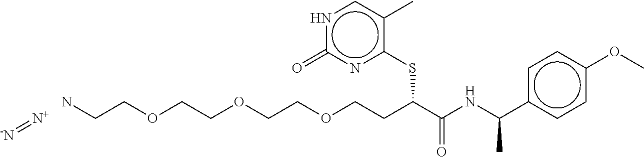 | I-262 |
| 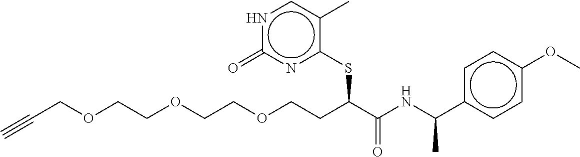 | I-263 |
| 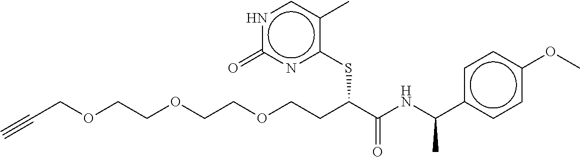 | I-264 |
| 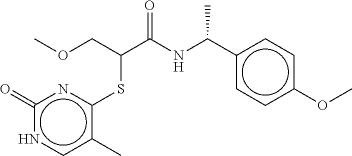 | I-265 |
| 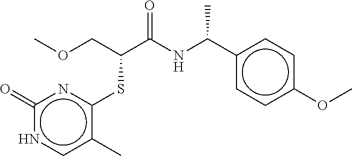 | I-266 |
| 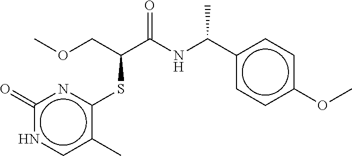 | I-267 |
| 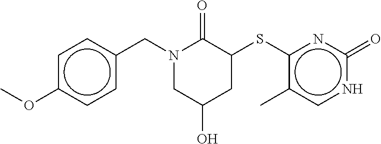 | I-268 |
| 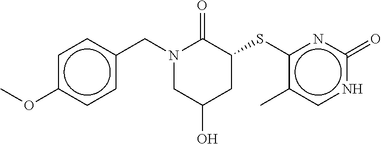 | I-269 |
| 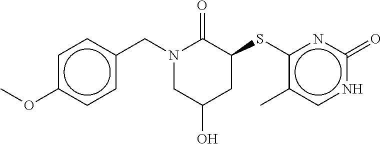 | I-270 |
| 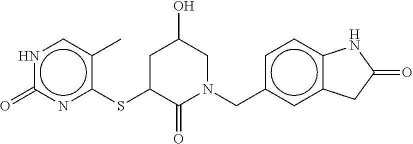 | I-271 |
| 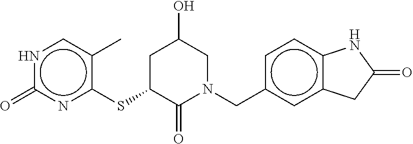 | I-272 |
| 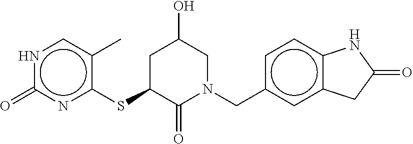 | I-273 |
| 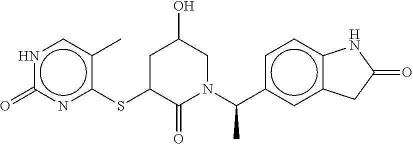 | I-274 |
| 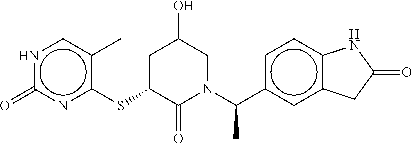 | I-275 |
| 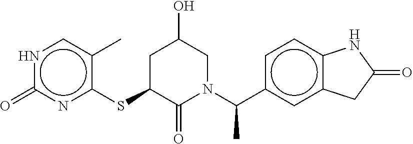 | I-276 |
| 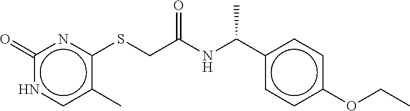 | I-277 |
| 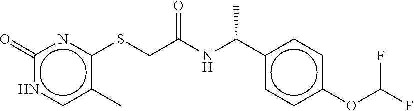 | I-278 |
| 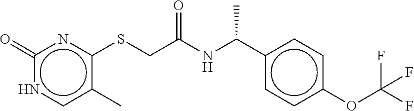 | I-279 |
| 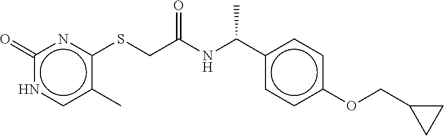 | I-280 |
| 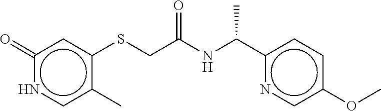 | I-281 |
| 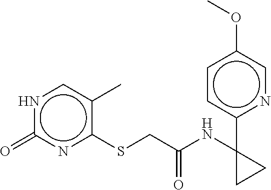 | I-282 |
| 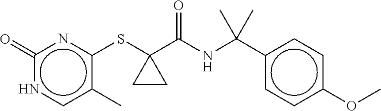 | I-283 |
| 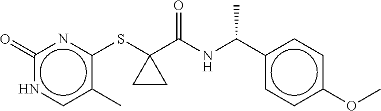 | I-284 |
| 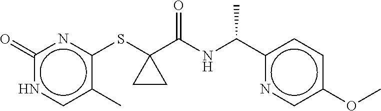 | I-285 |
| 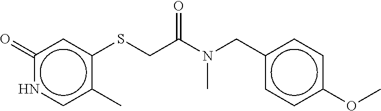 | I-286 |
| 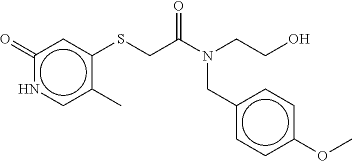 | I-287 |
| 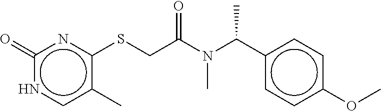 | I-288 |
| 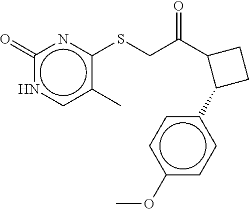 | I-289 |
| 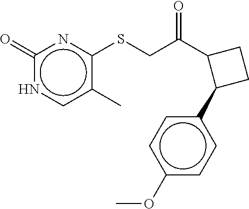 | I-290 |
| 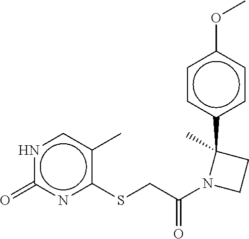 | I-291 |
| 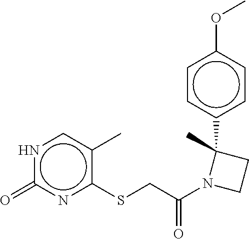 | I-292 |
| 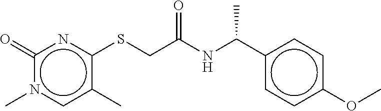 | I-293 |
| 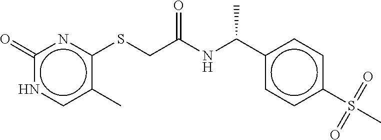 | I-294 |
| 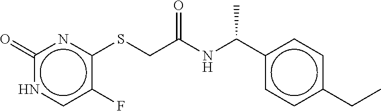 | I-295 |
| 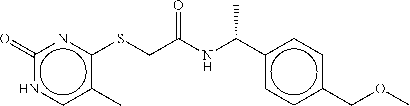 | I-296 |
| 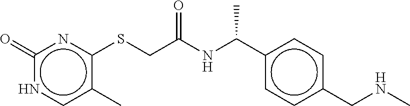 | I-297 |
| 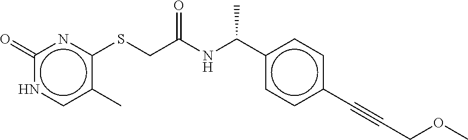 | I-298 |
| 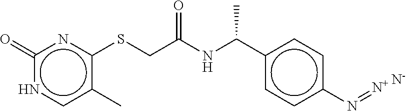 | I-299 |
| 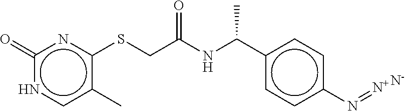 | I-300 |
| 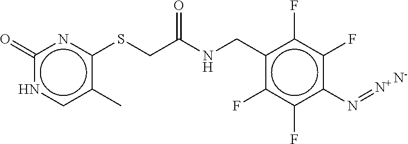 | I-301 |
| 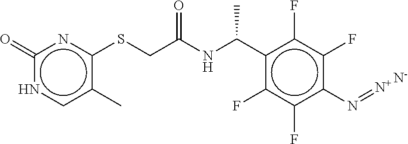 | I-302 |
| 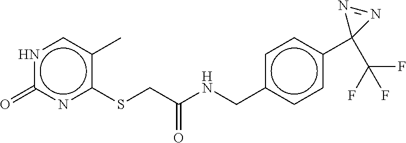 | I-303 |
| 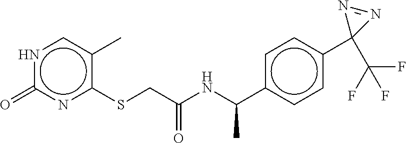 | I-304 |
| 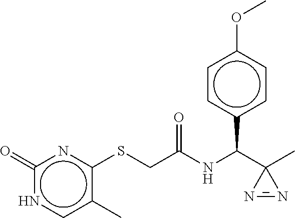 | I-305 |
| 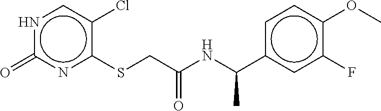 | I-306 |
| 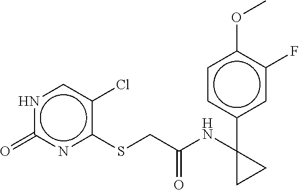 | I-307 |
| 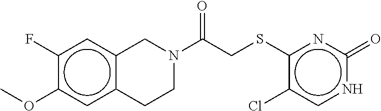 | I-308 |
| 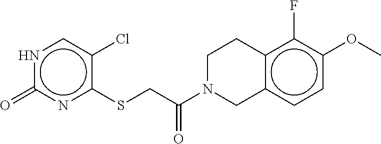 | I-309 |
| 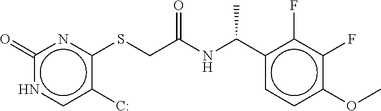 | I-310 |
| 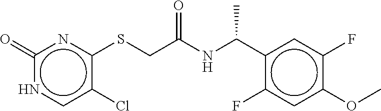 | I-311 |
| 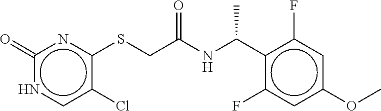 | I-312 |
| 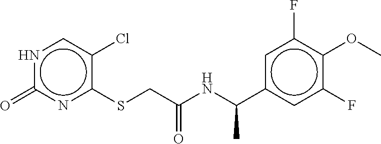 | I-313 |
| 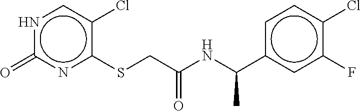 | I-314 |
| 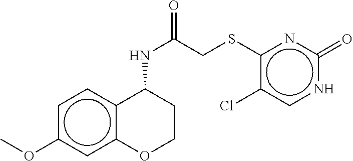 | I-315 |
| 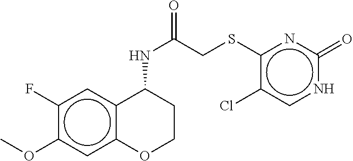 | I-316 |
| 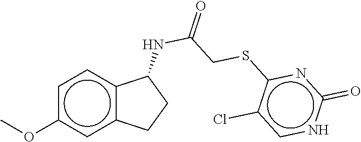 | I-317 |
| 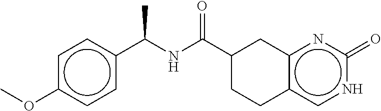 | I-318 |
| 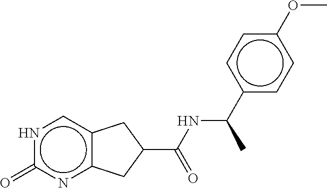 | I-319 |
| 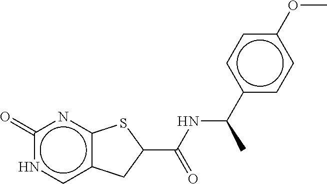 | I-320 |
| 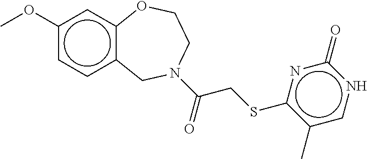 | I-321 |

TABLE 1B
Exemplary Compounds I-322 to I-442

| | |
|---|---|
| 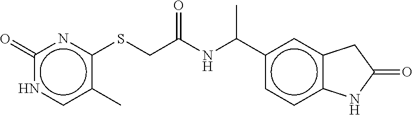 | I-322 |
| 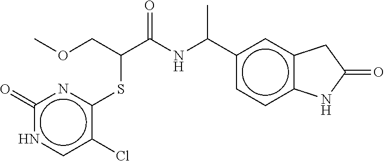 | I-323 |
| 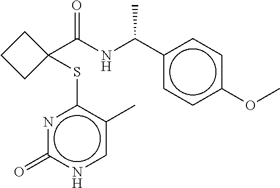 | I-324 |
| 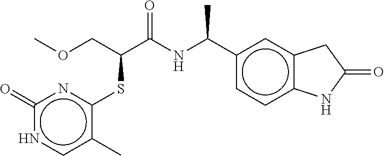 | I-325 |
| 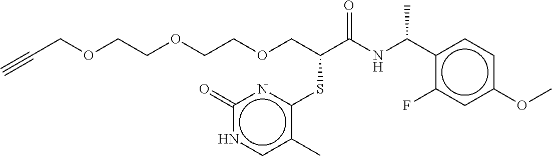 | I-326 |
| 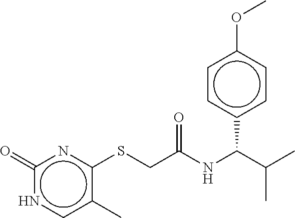 | I-327 |
| 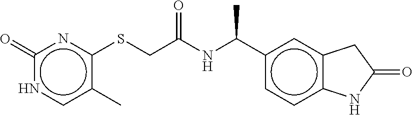 | I-328 |
| 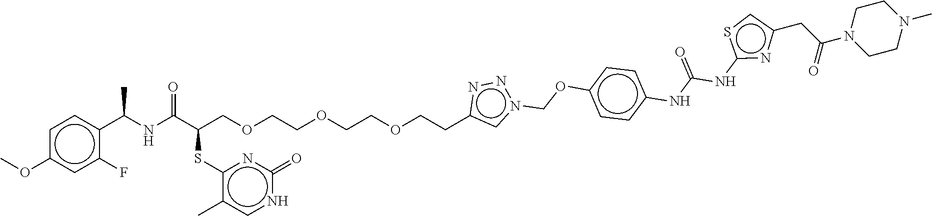 | I-329 |
| 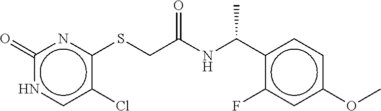 | I-330 |
| 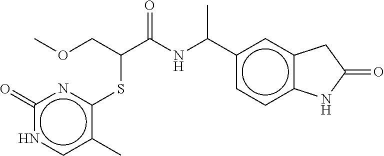 | I-331 |
| 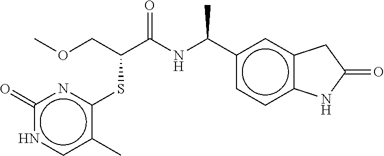 | I-332 |
| 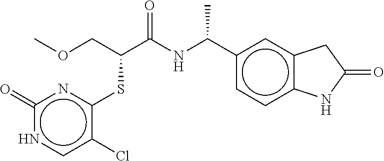 | I-333 |
| 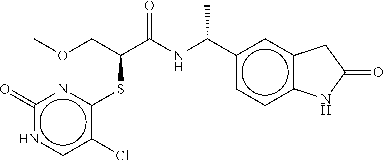 | I-334 |
| 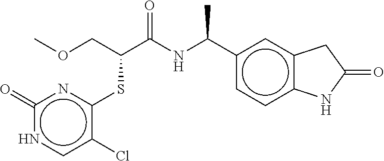 | I-335 |
| 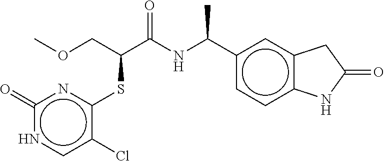 | I-336 |
| 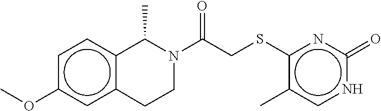 | I-337 |
| 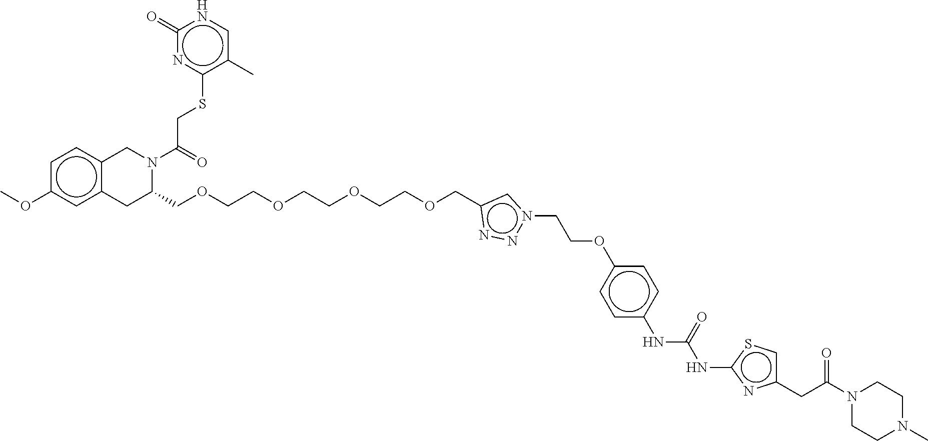 | I-338 |
| 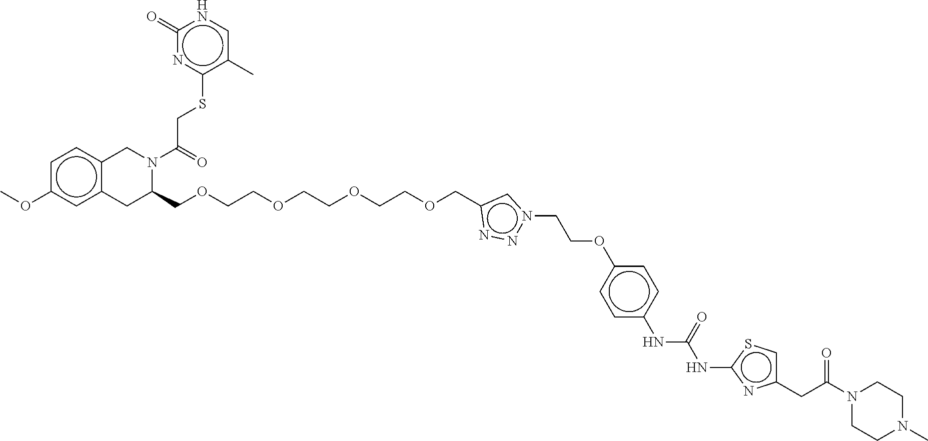 | I-339 |
| 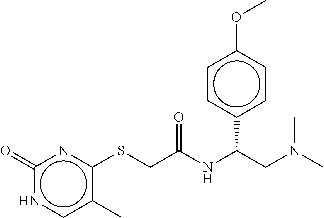 | I-340 |
| 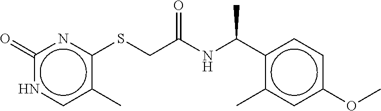 | I-341 |
| 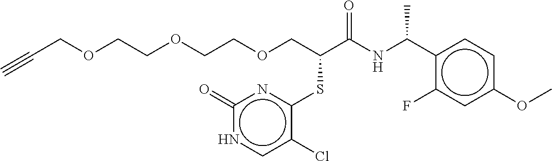 | I-342 |
| 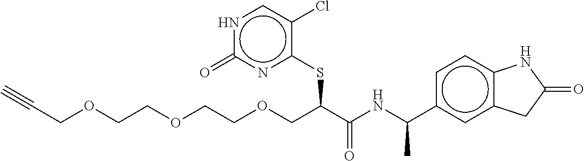 | I-343 |
| 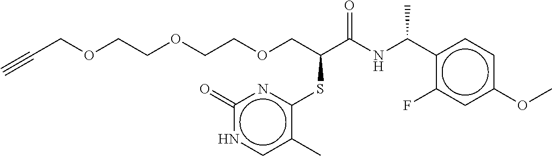 | I-344 |
| 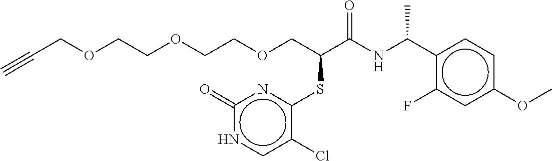 | I-345 |
| 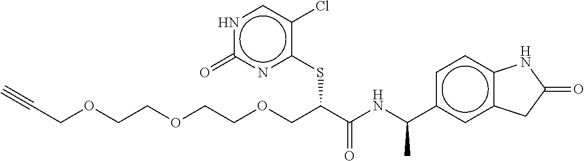 | I-346 |
| 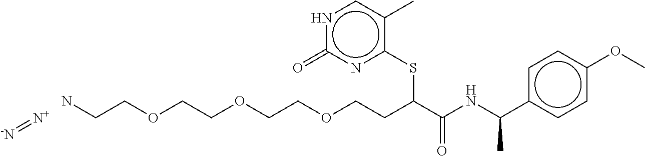 | I-347 |
| 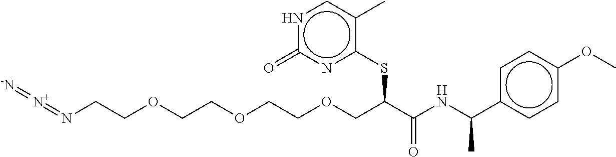 | I-348 |
| 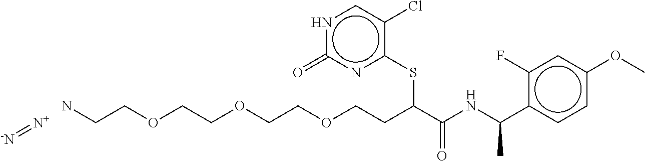 | I-349 |
| 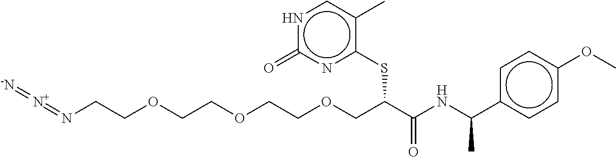 | I-350 |
| 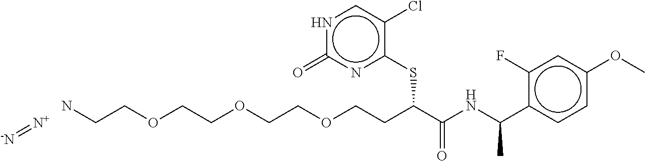 | I-351 |
| 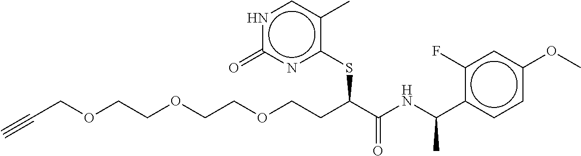 | I-352 |
| 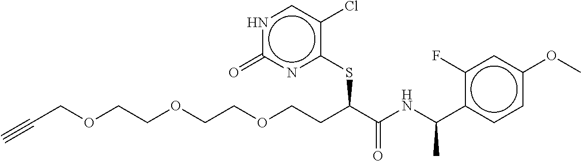 | I-353 |
| 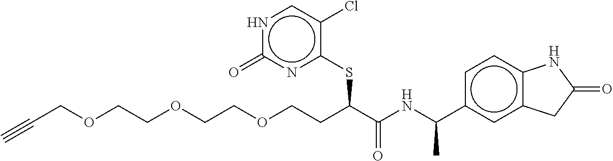 | I-354 |
| 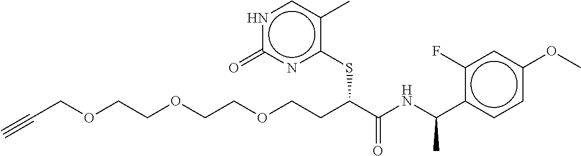 | I-355 |
| 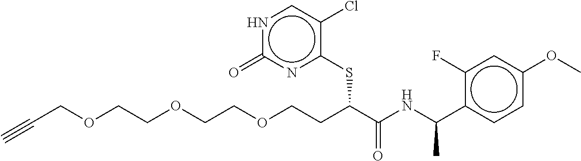 | I-356 |
| 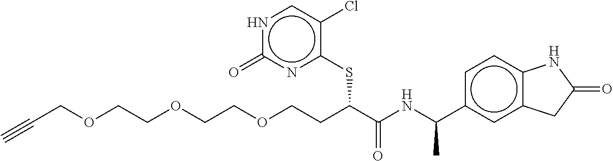 | I-357 |
| 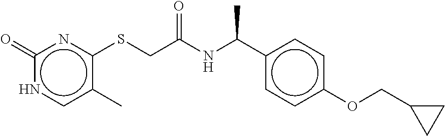 | I-358 |
| 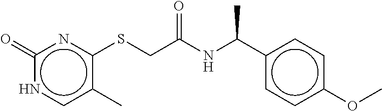 | I-359 |
| 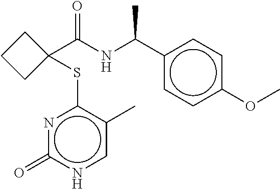 | I-360 |
| 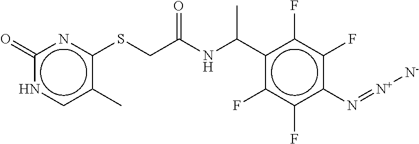 | I-361 |
| 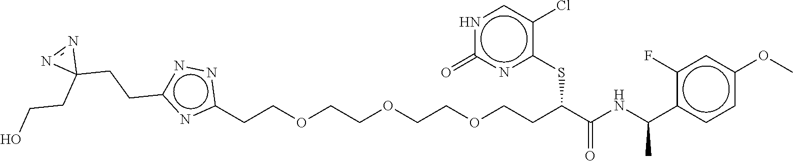 | I-362 |
| 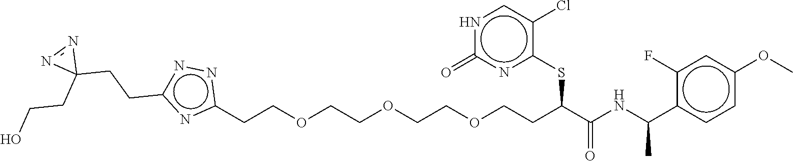 | I-363 |
| 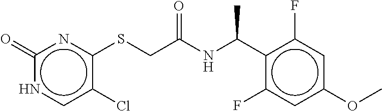 | I-364 |
| 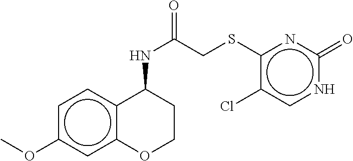 | I-365 |
| 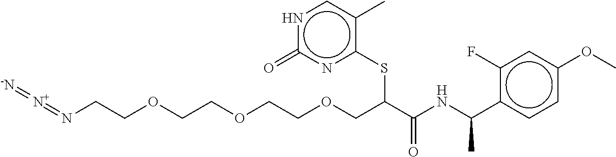 | I-366 |
| 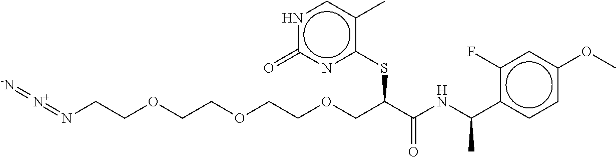 | I-367 |
| 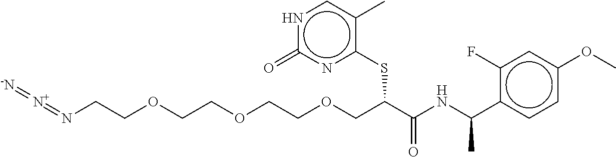 | I-368 |
| 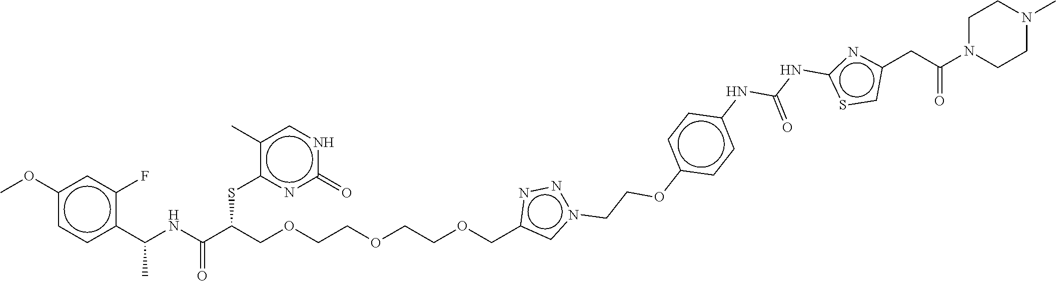 | I-369 |
| 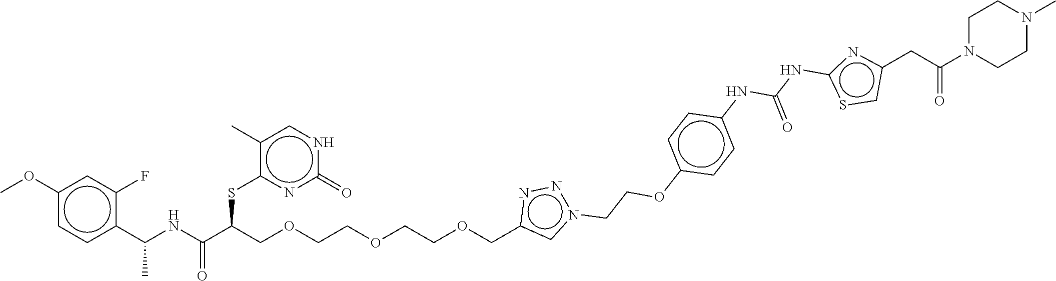 | I-370 |
| 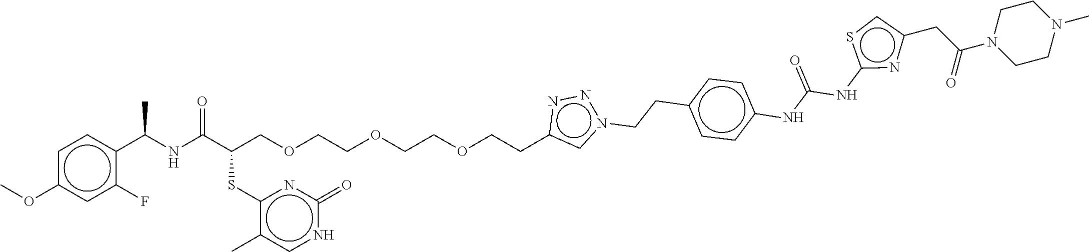 | I-371 |
| 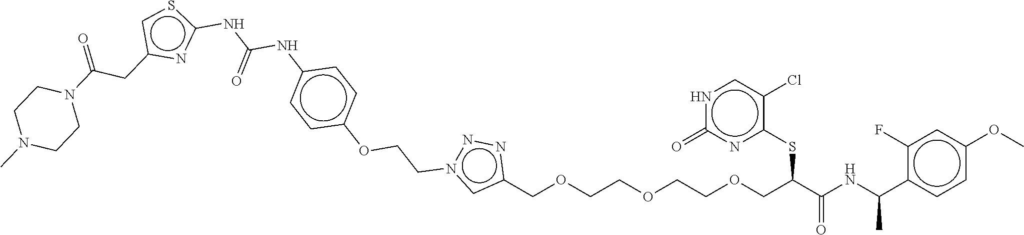 | I-372 |
| 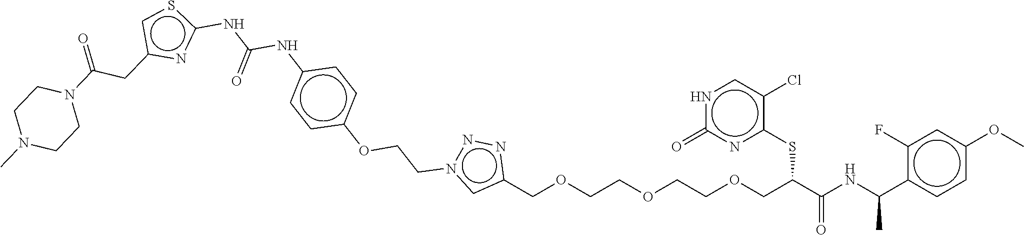 | I-373 |
| 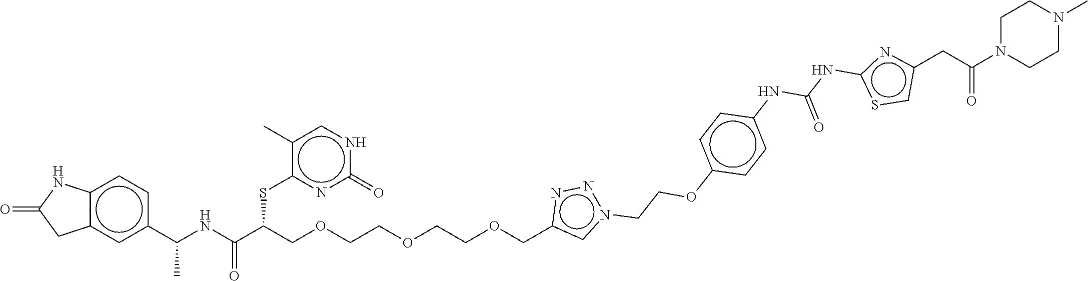 | I-374 |
| 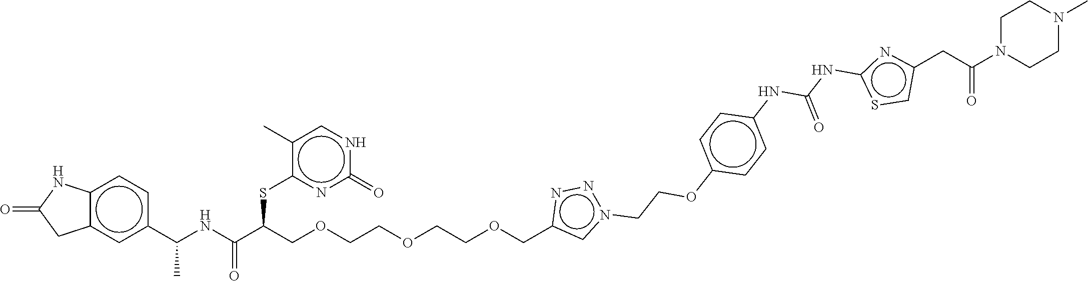 | I-375 |
| 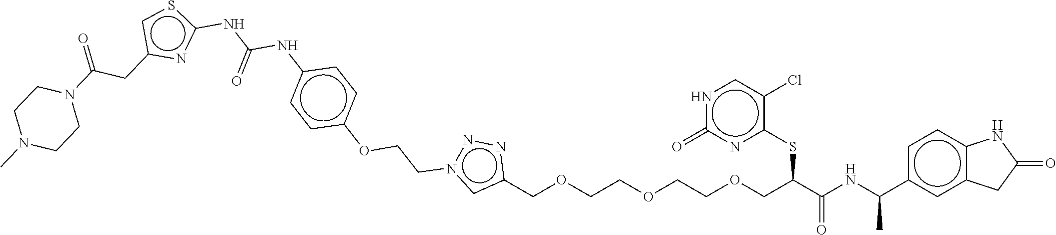 | I-376 |
| 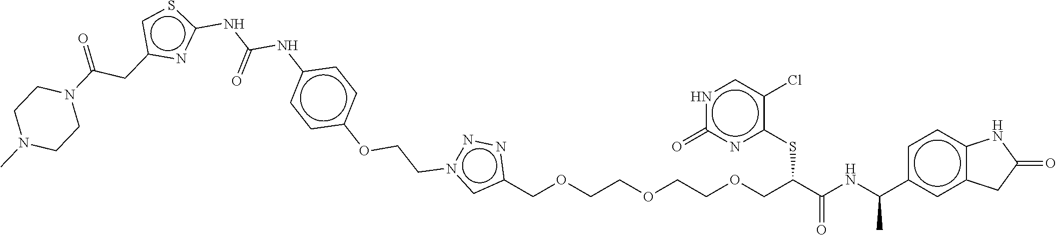 | I-377 |
| 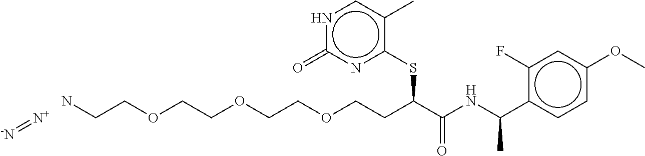 | I-378 |
| 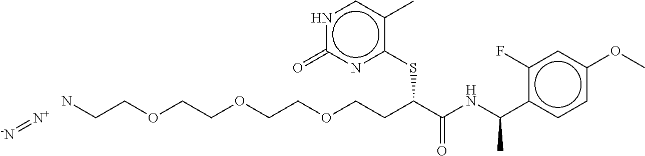 | I-379 |
| 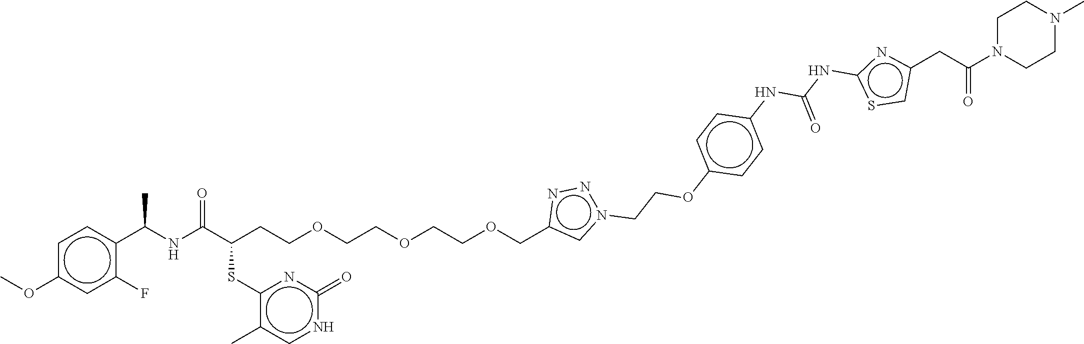 | I-380 |
| 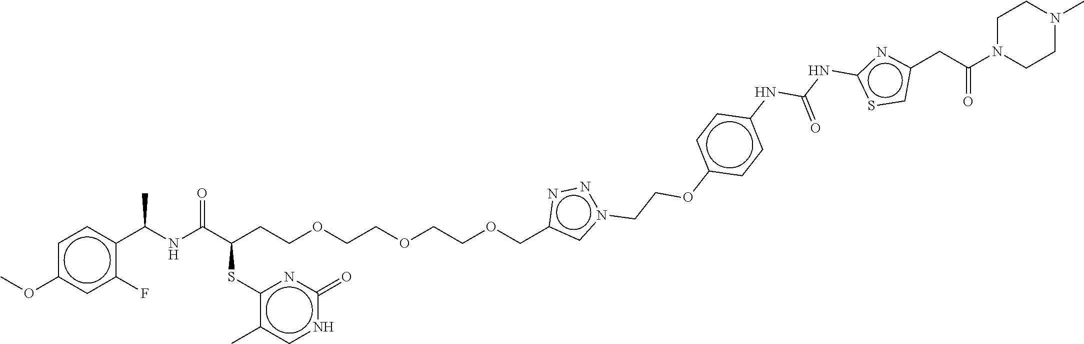 | I-381 |
| 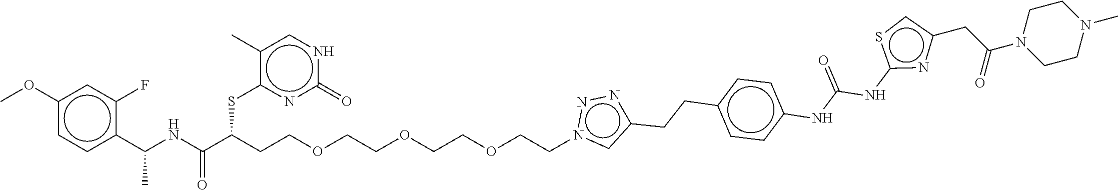 | I-382 |
| 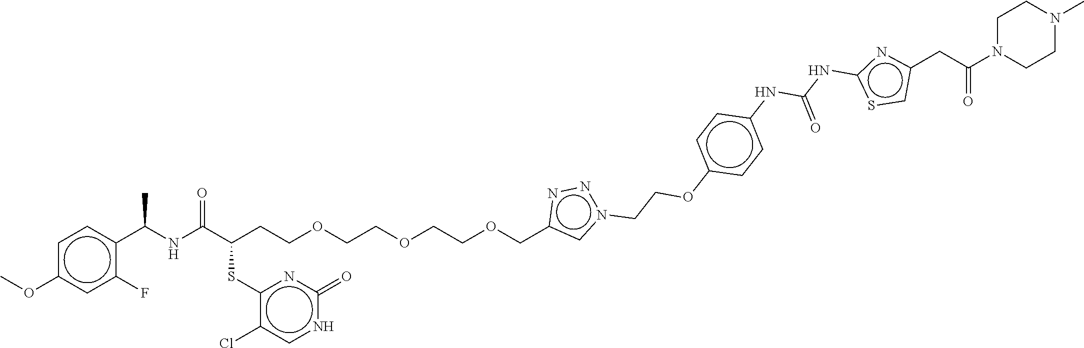 | I-383 |
| 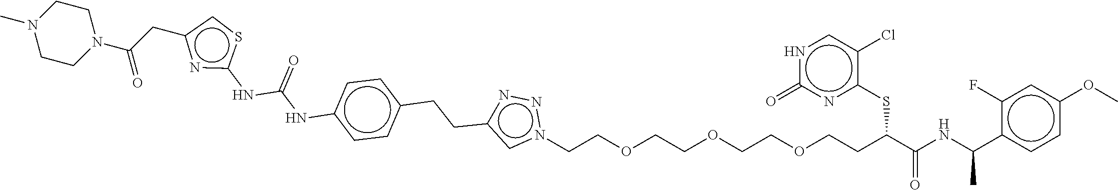 | I-384 |
| 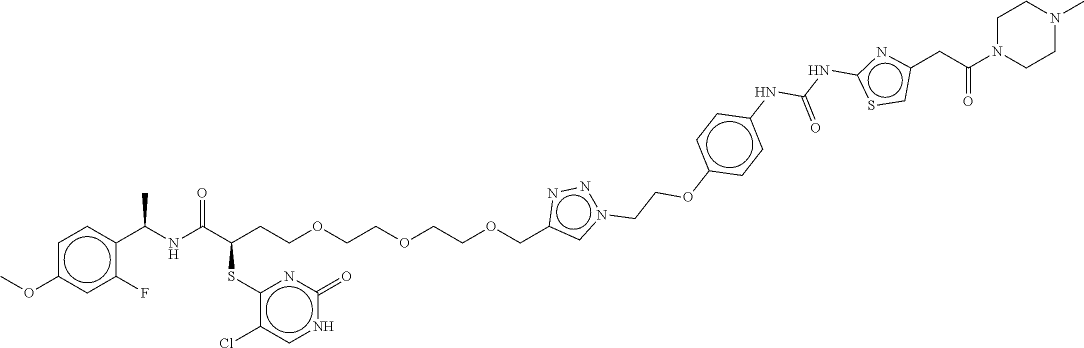 | I-385 |
| 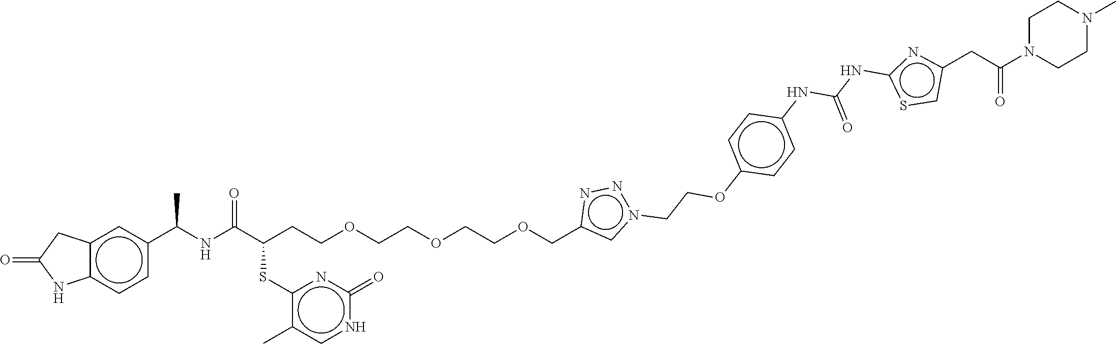 | I-386 |
| 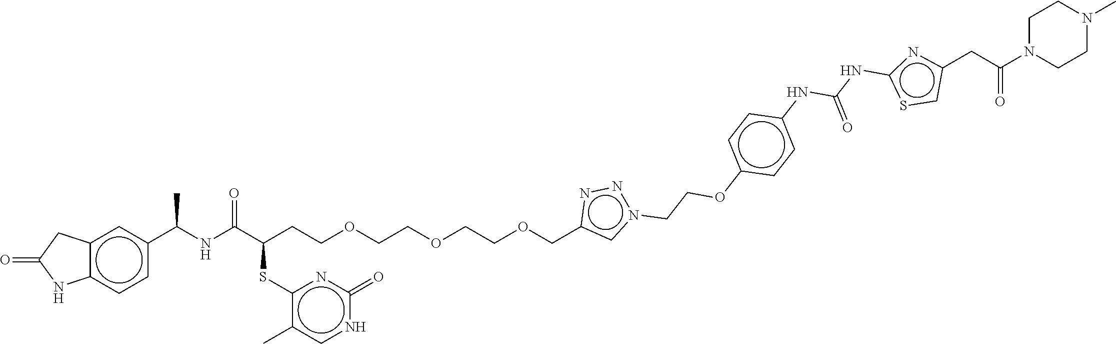 | I-387 |
| 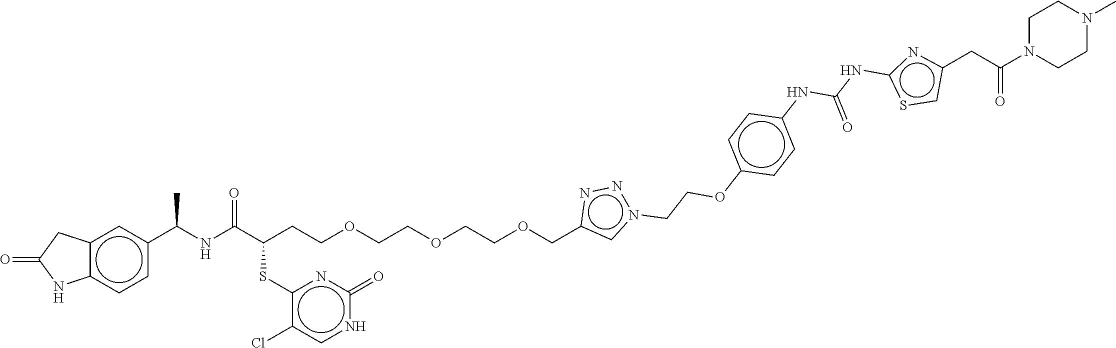 | I-388 |
| 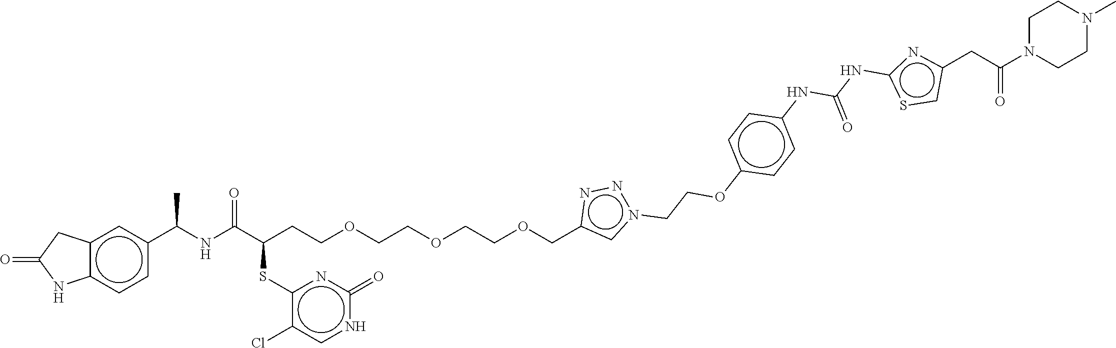 | I-389 |
| 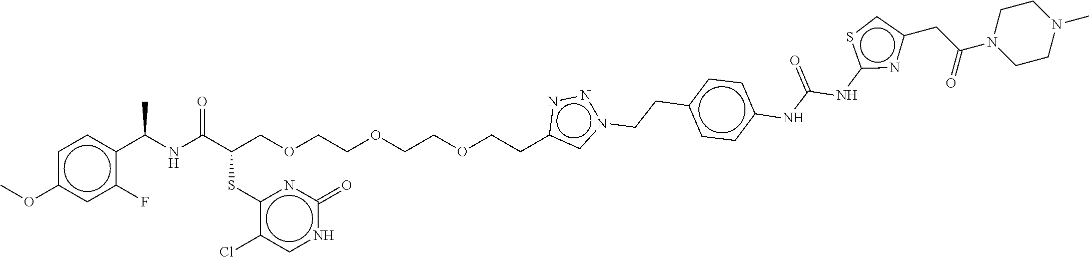 | I-390 |
| 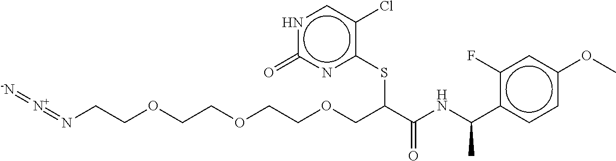 | I-391 |
| 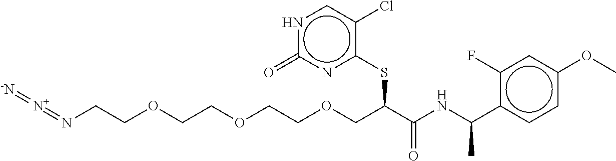 | I-392 |
| 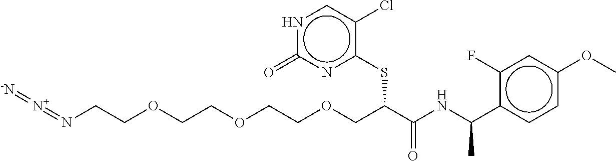 | I-393 |
| 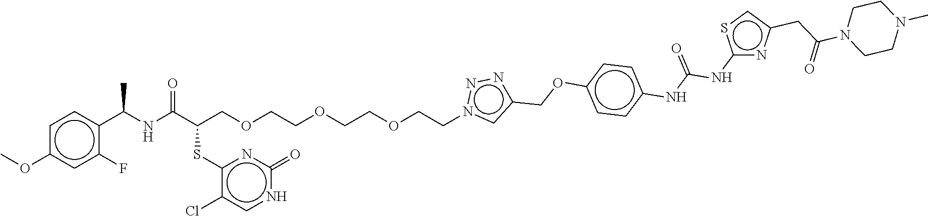 | I-394 |
| 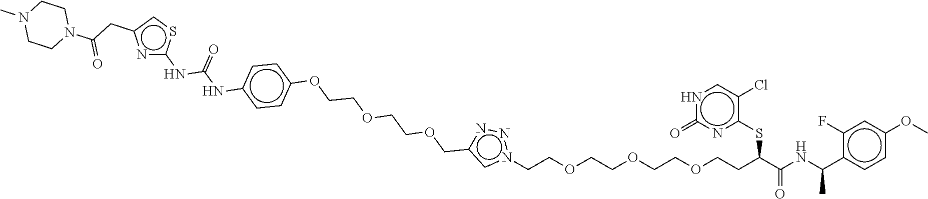 | I-395 |
| 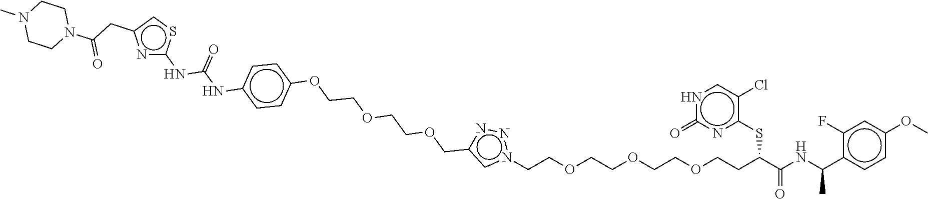 | I-396 |
| 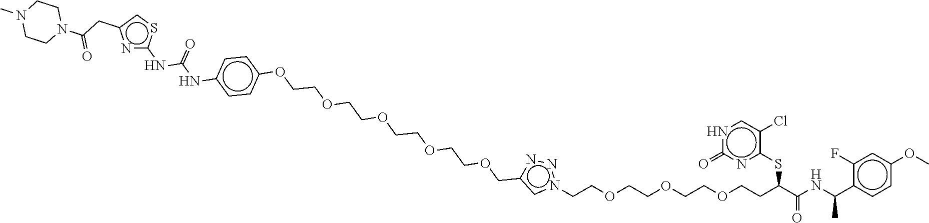 | I -397 |
| 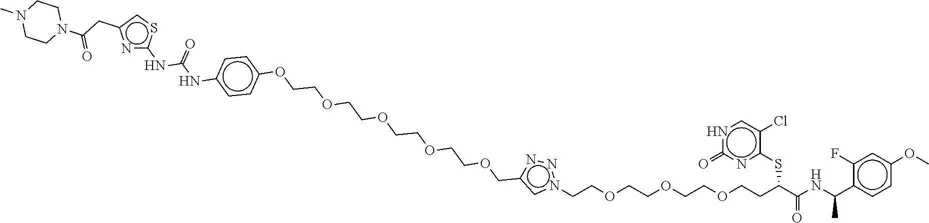 | I-398 |
| 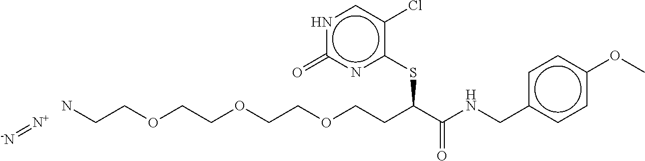 | I-399 |
| 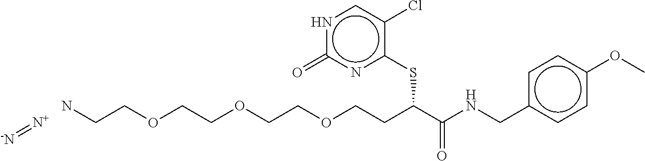 | I-400 |
| 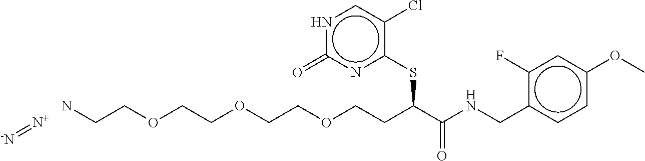 | I-401 |
| 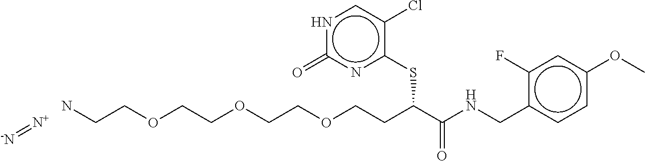 | I-402 |
| 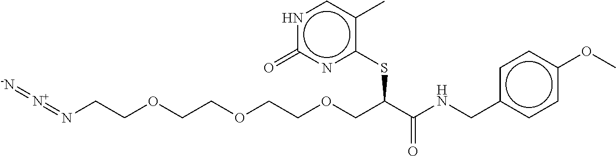 | I-403 |
| 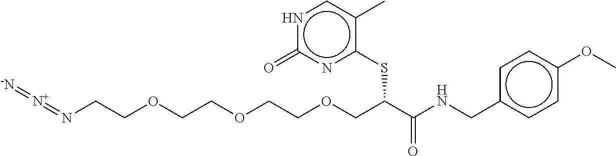 | I-404 |
| 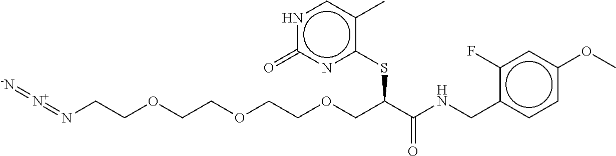 | I-405 |
| 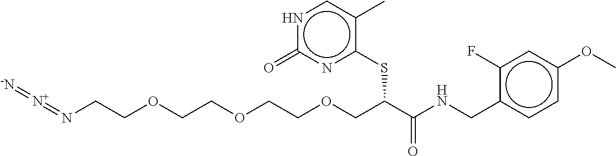 | I-406 |
| 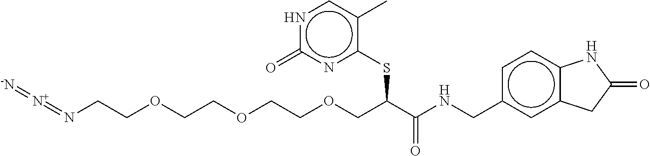 | I-407 |
| 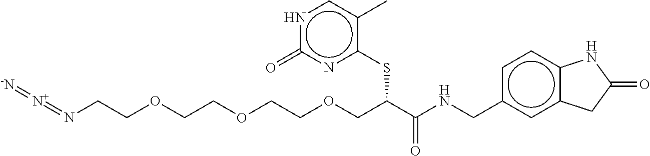 | I-408 |
| 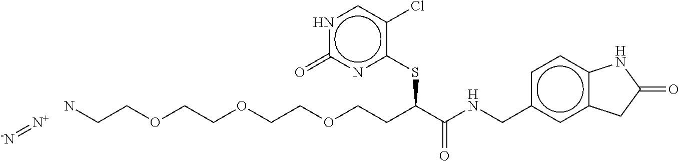 | I-409 |
| 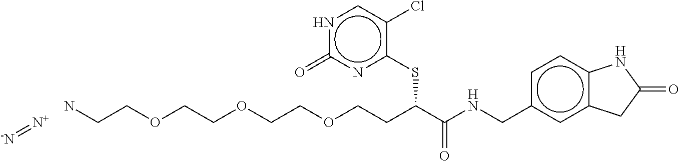 | I-410 |
| 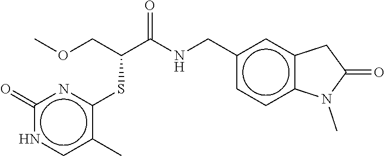 | I-411 |
| 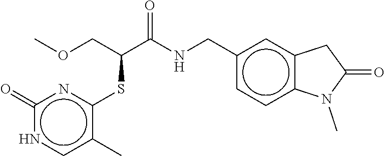 | I-412 |
| 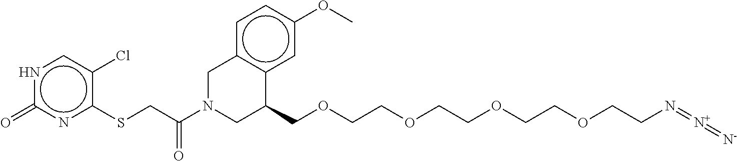 | I-413 |
| 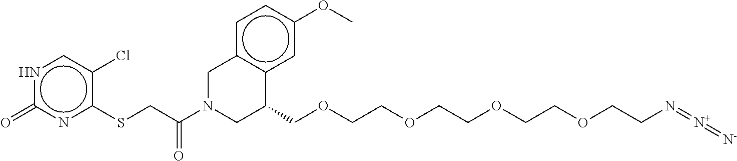 | I-414 |
| 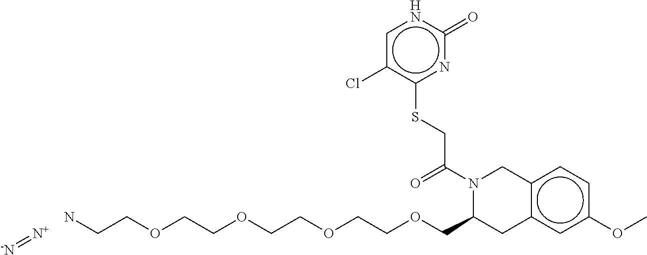 | I-415 |
| 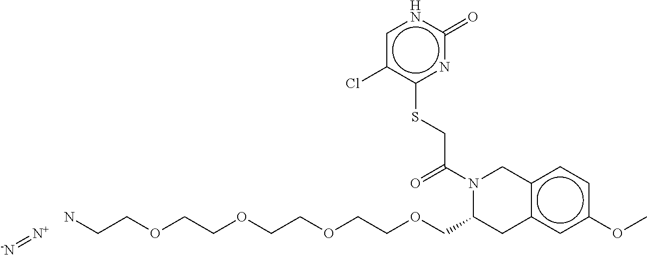 | I-416 |
| 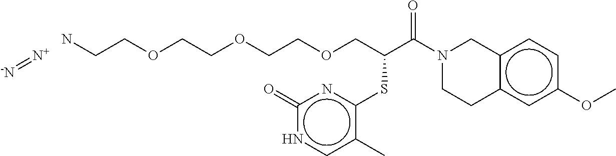 | I-417 |
| 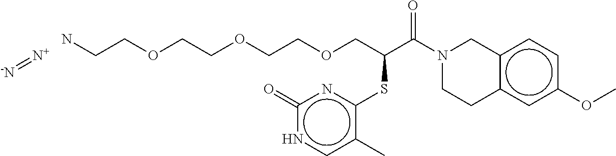 | I-418 |
| 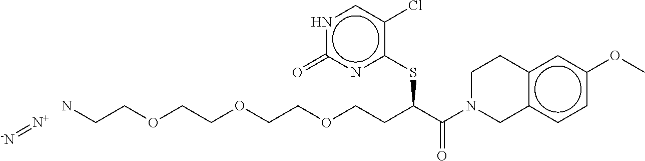 | I-419 |
| 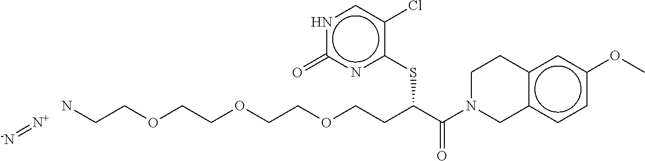 | I-420 |
| 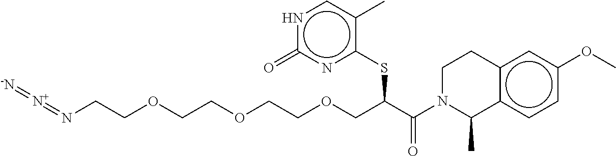 | I-421 |
| 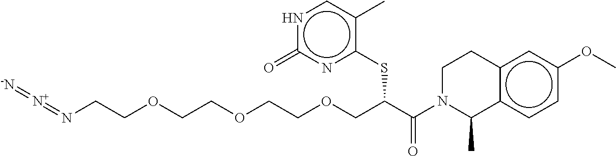 | I-422 |
| 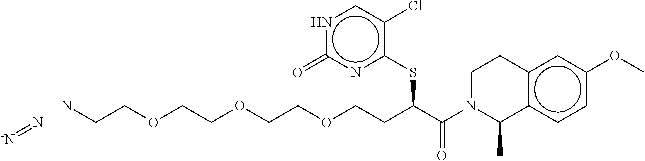 | I-423 |
| 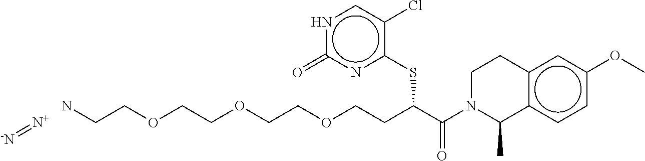 | I-424 |
| 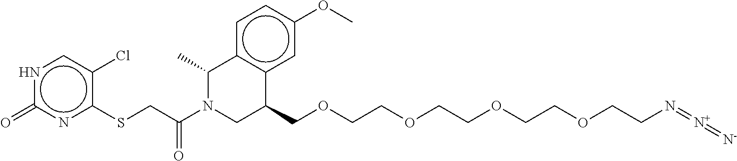 | I-425 |
| 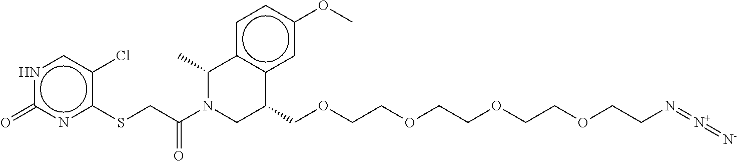 | I-426 |
| 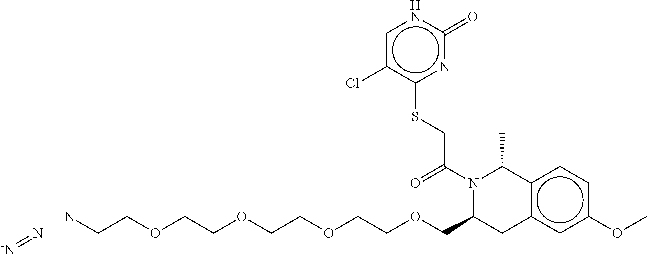 | I-427 |
| 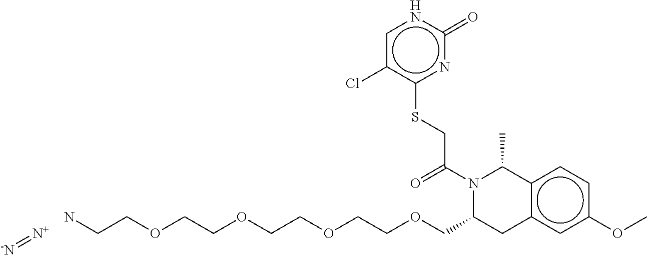 | I-428 |
| 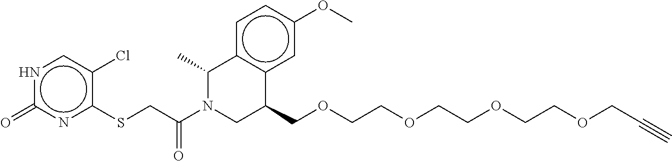 | I-429 |
| 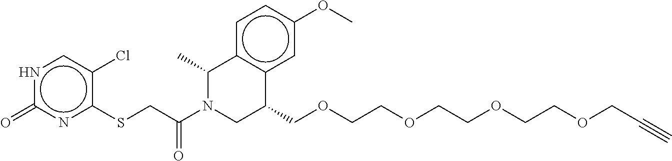 | I-430 |
| 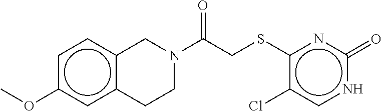 | I-431 |
| 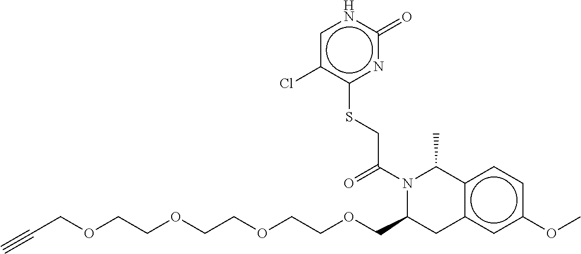 | I-432 |
| 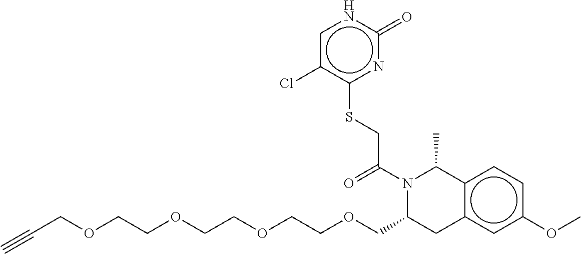 | I-433 |
| 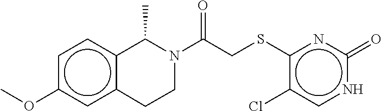 | I-434 |
| 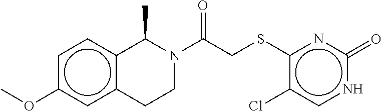 | I-435 |
| 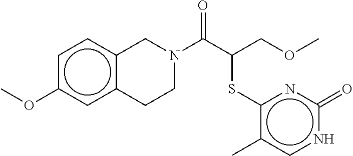 | I-436 |
| 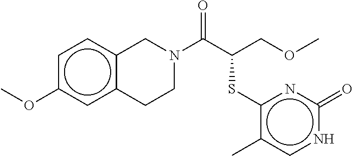 | I-437 |
| 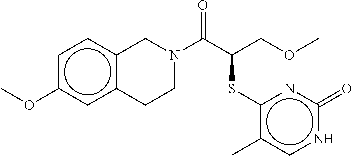 | I-438 |
| 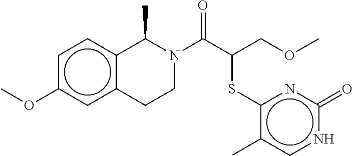 | I-439 |
| 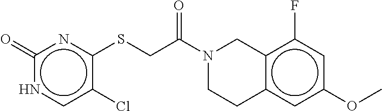 | I-440 |
| 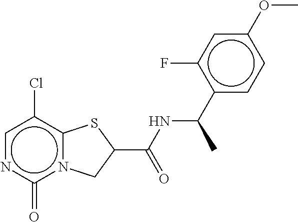 | I-441 |
| 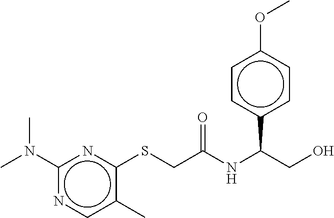 | I-442 |

TABLE 1C
Exemplary Compounds

| | |
|---|---|
| 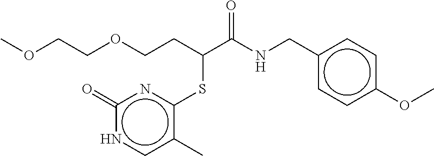 | I-443 |
| 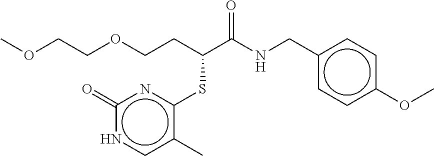 | I-444 |
| 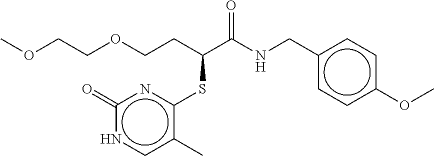 | I-445 |
| 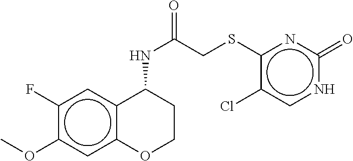 | I-446 |
| 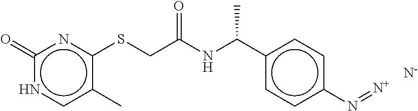 | I-447 |
| 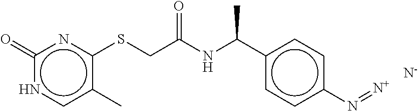 | I-448 |
| 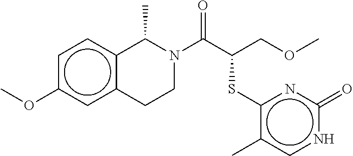 | I-449 |
| 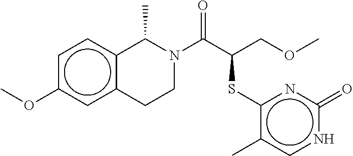 | I-450 |
| 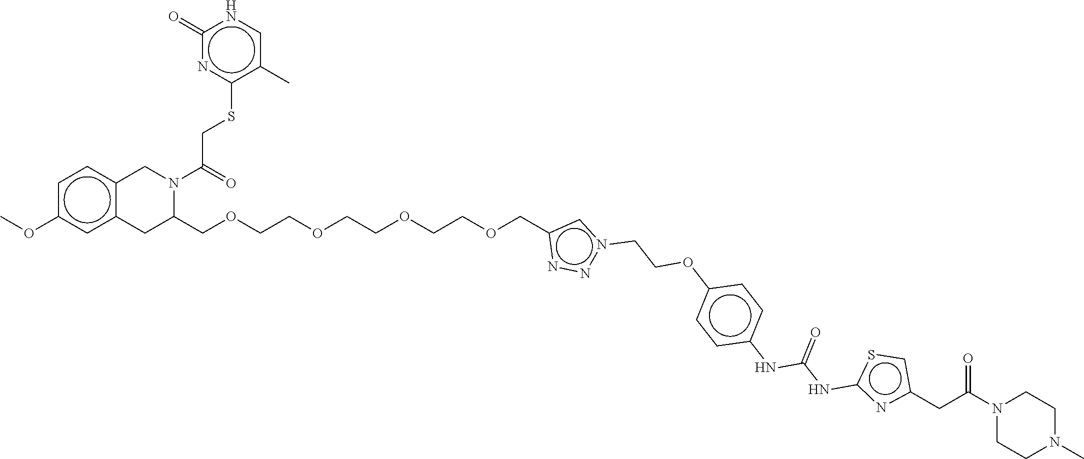 | I-451 |
| 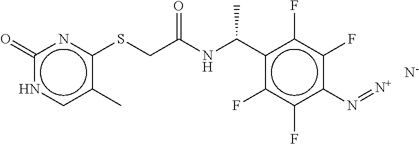 | I-452 |
| 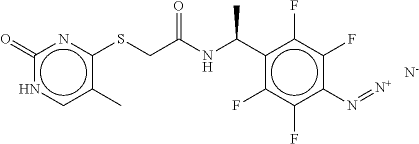 | I-453 |
| 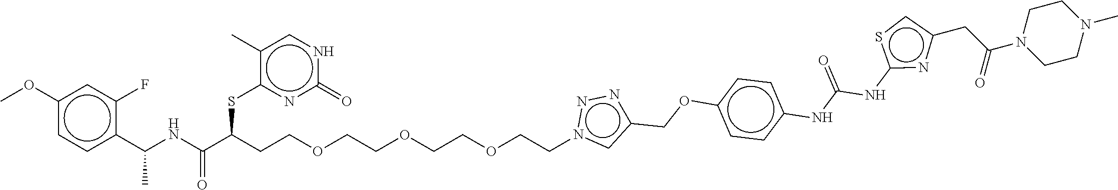 | I-454 |
| 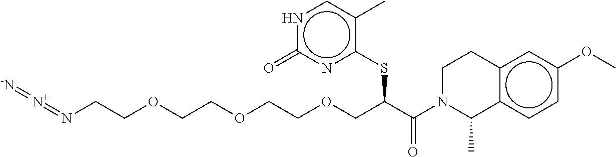 | I-455 |
| 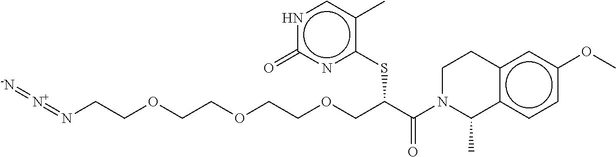 | I-456 |
| 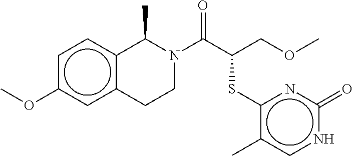 | I-457 |
| 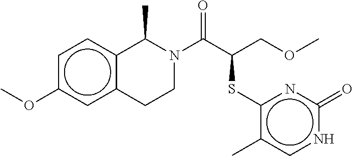 | I-458 |
| 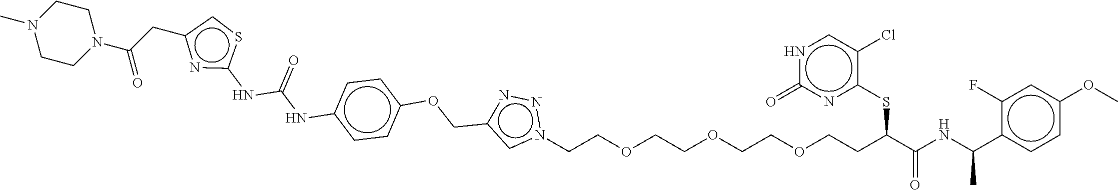 | I-459 |
| 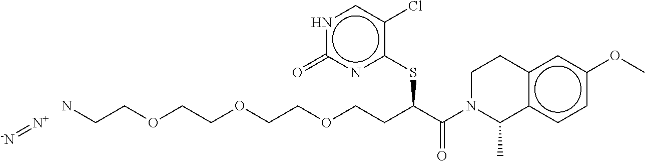 | I-460 |
| 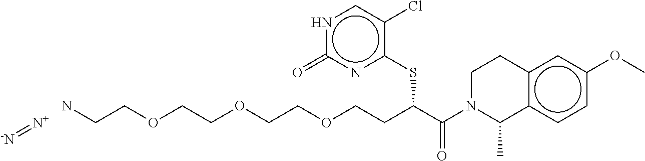 | I-461 |
| 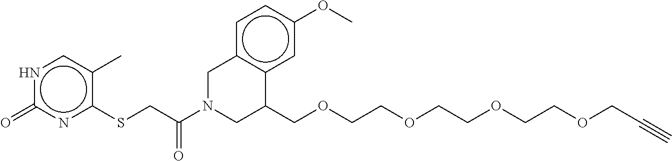 | I-462 |
| 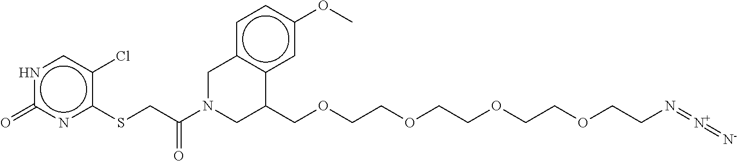 | I-463 |
| 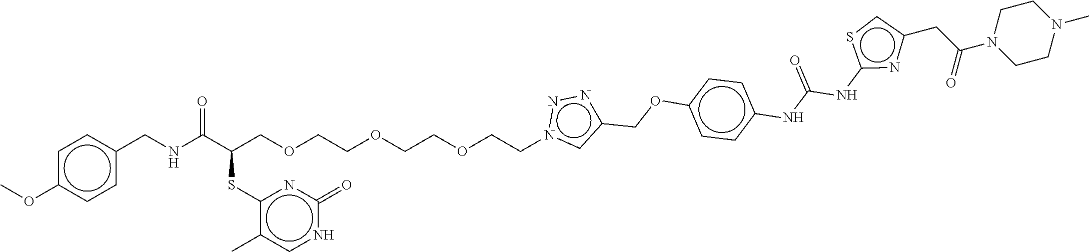 | I-464 |
| 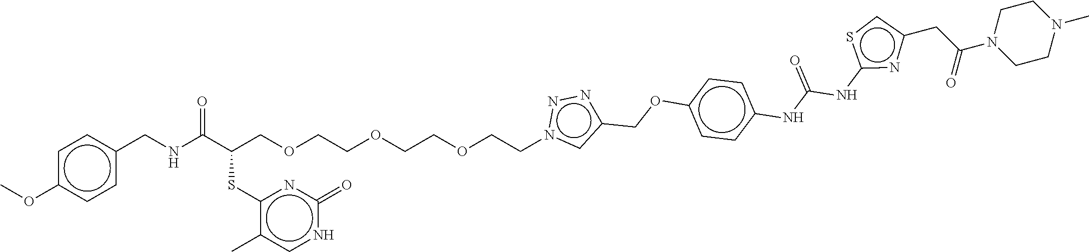 | I-465 |
| 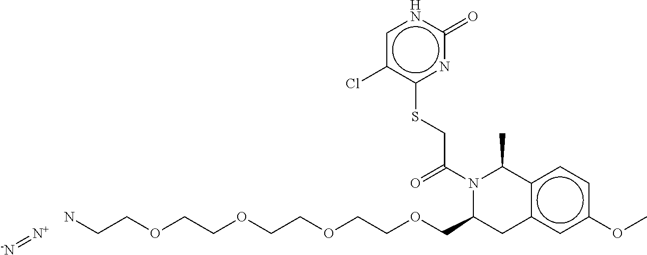 | I-466 |
| 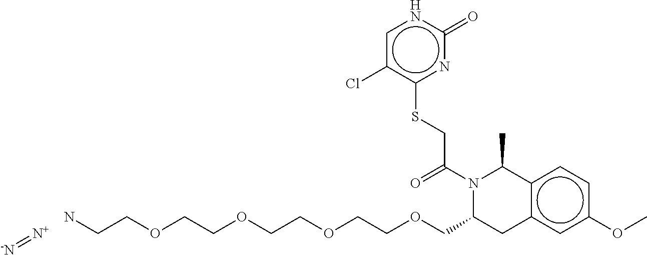 | I-467 |
| 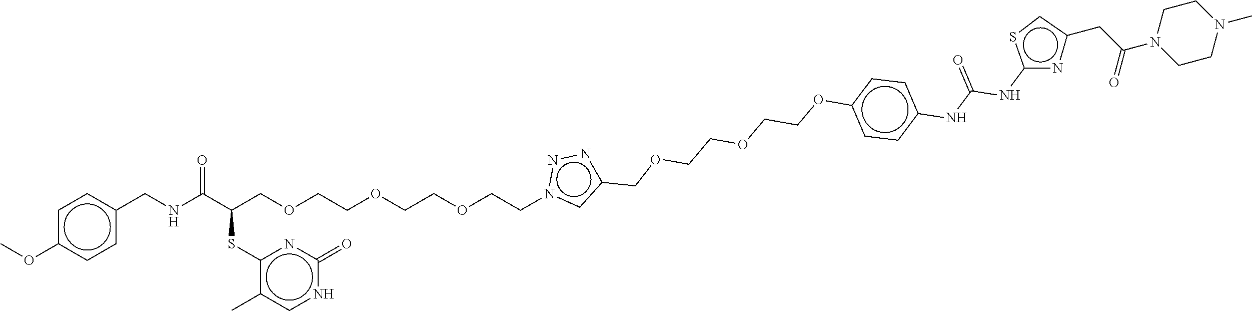 | I-468 |
| 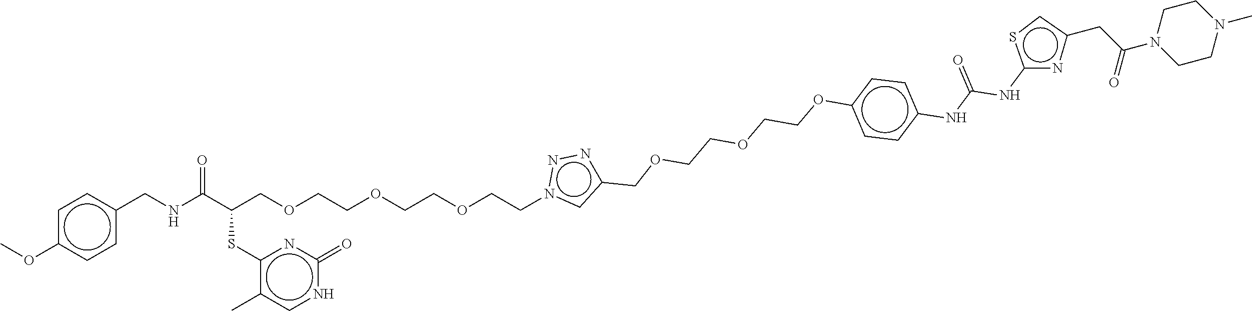 | I-469 |
| 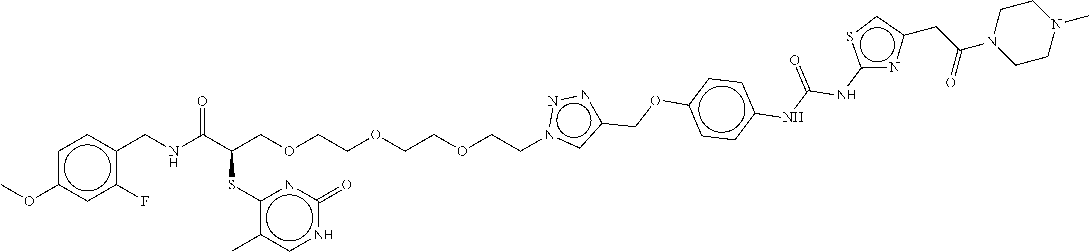 | I-470 |
| 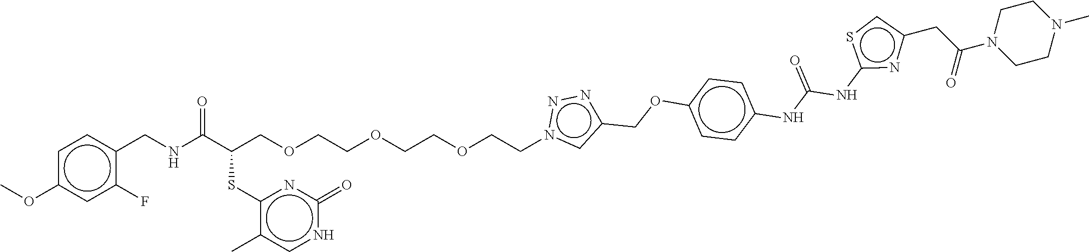 | I-471 |
| 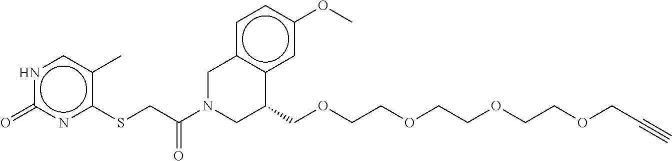 | I-472 |
| 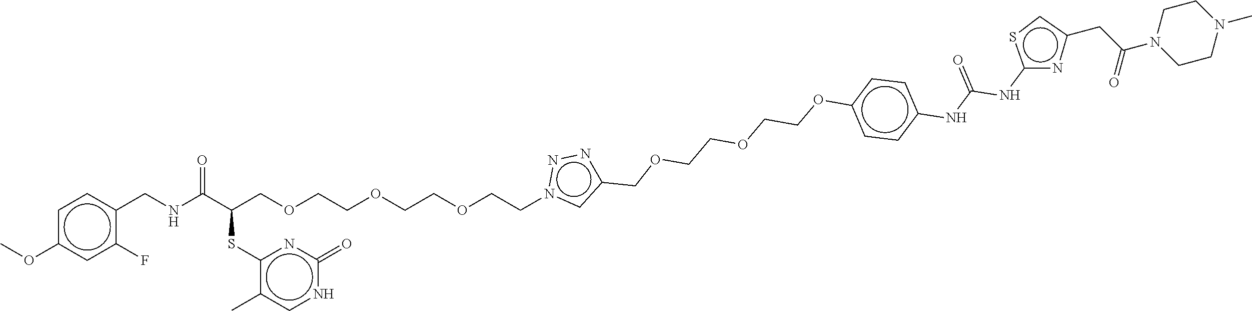 | I-473 |
| 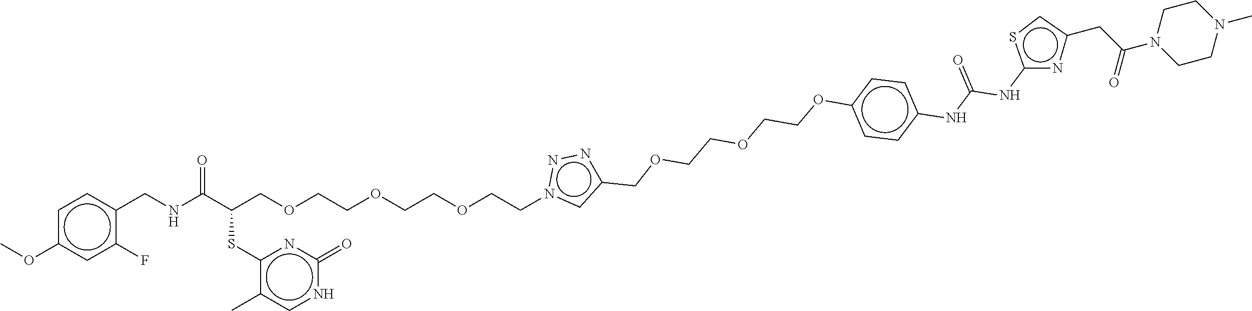 | I-474 |
| 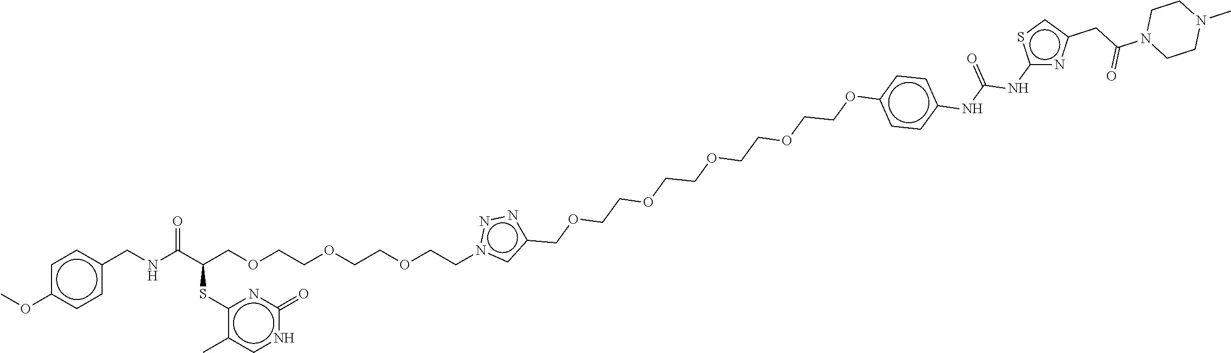 | I-475 |
| 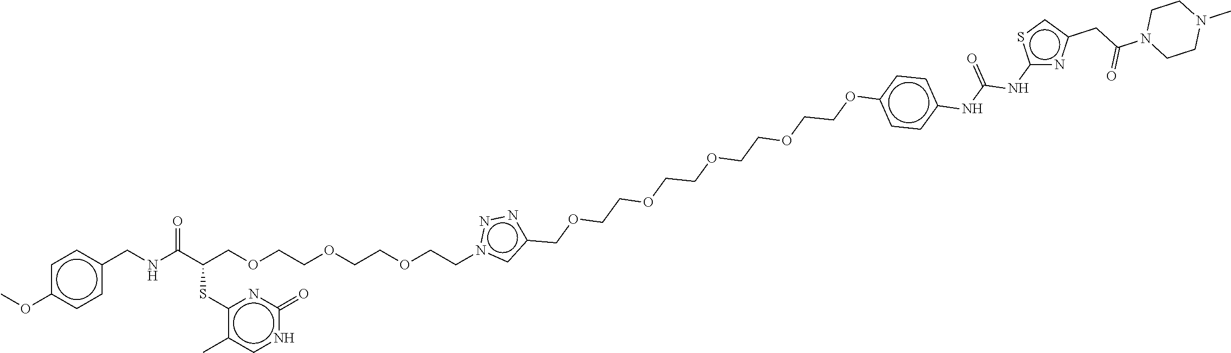 | I-476 |
| 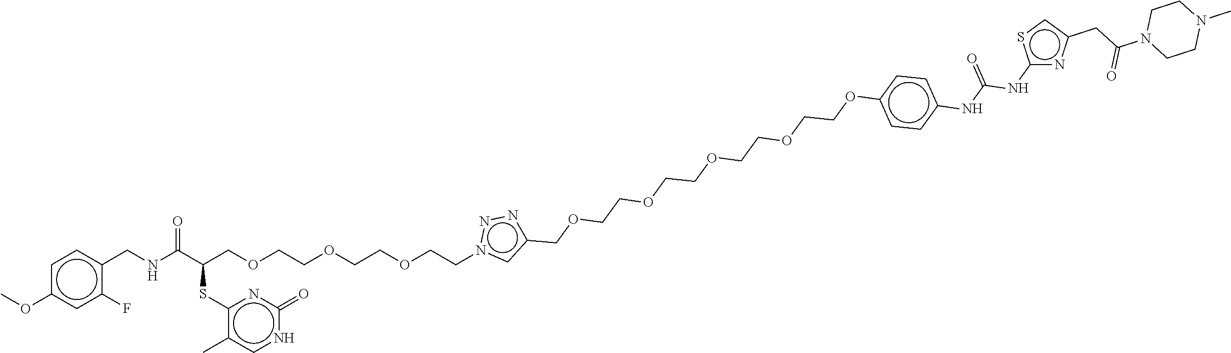 | I-477 |
| 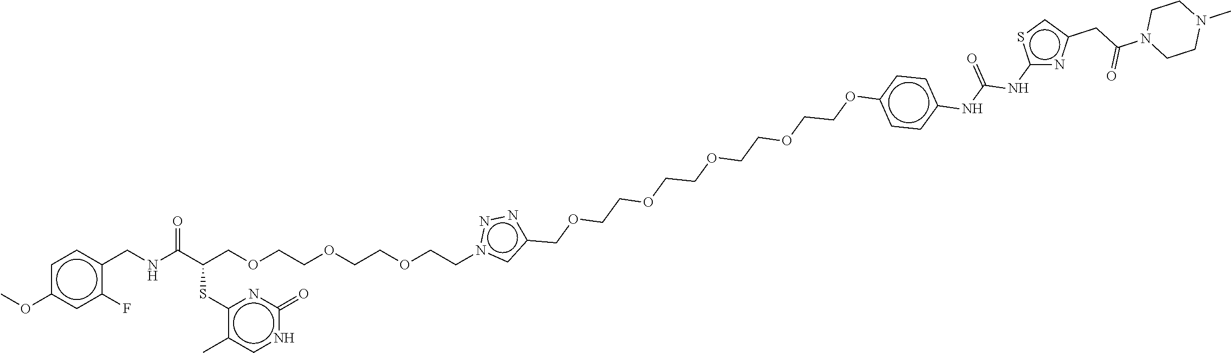 | I-478 |
| 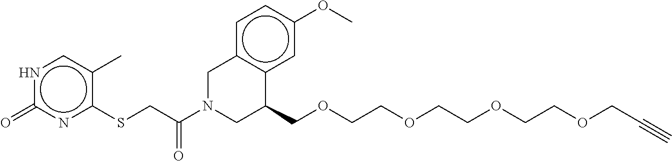 | I-479 |
| 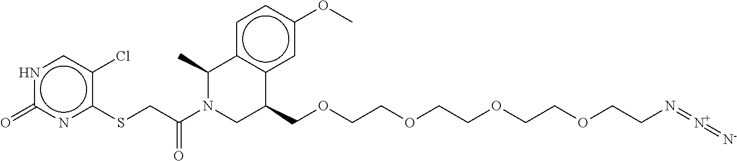 | I-480 |
| 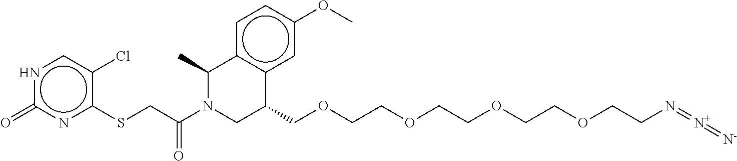 | I-481 |
| 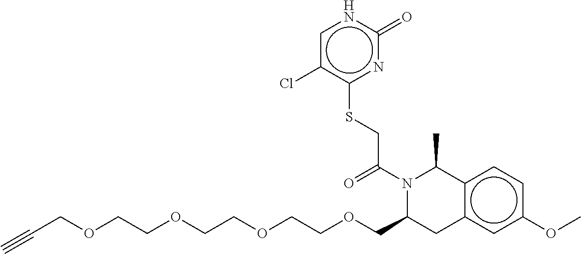 | I-482 |
| 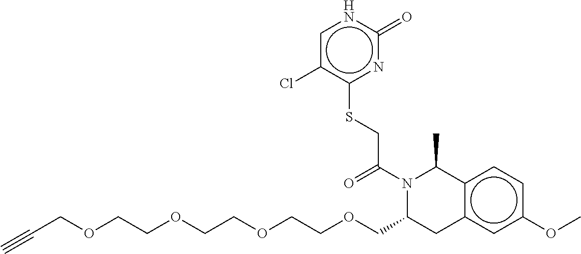 | I-483 |
| 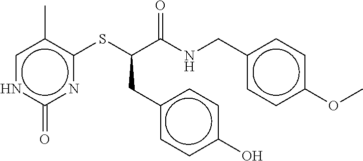 | I-484 |
| 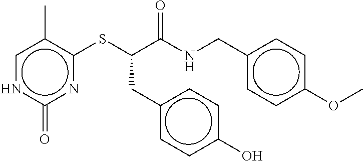 | I-485 |
| 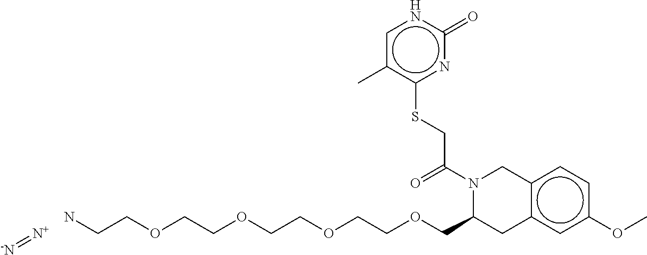 | I-486 |
| 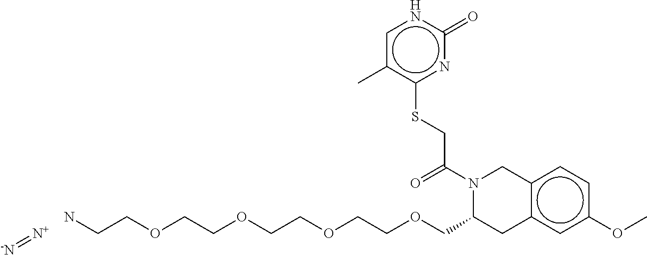 | I-487 |
| 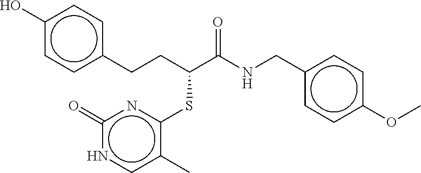 | I-488 |
| 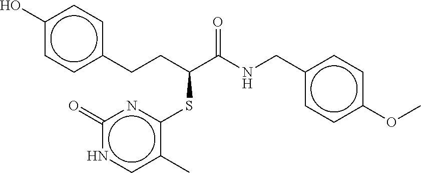 | I-489 |
| 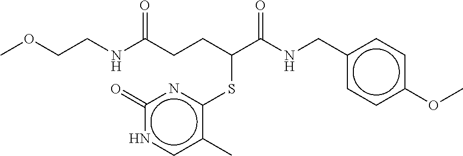 | I-490 |
| 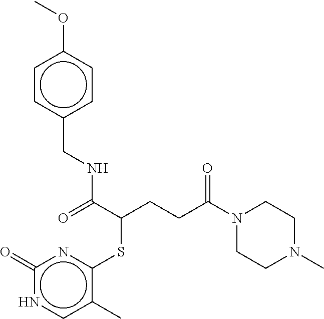 | I-491 |
| 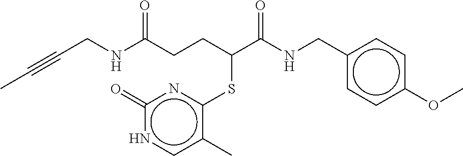 | I-492 |
| 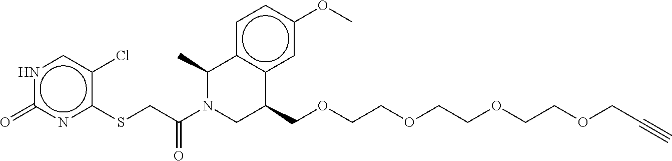 | I-493 |
| 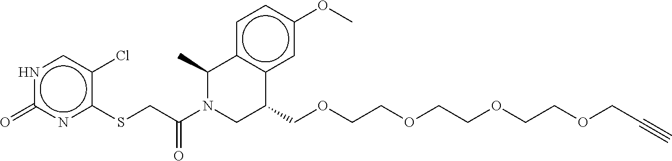 | I-494 |
| 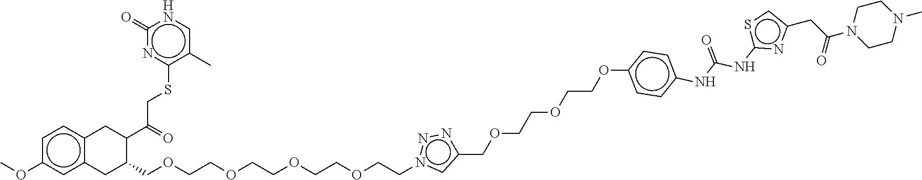 | I-495 |
| 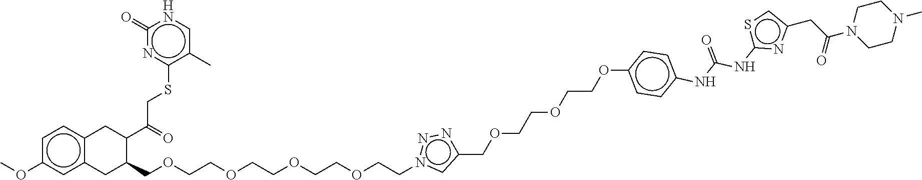 | I-496 |
| 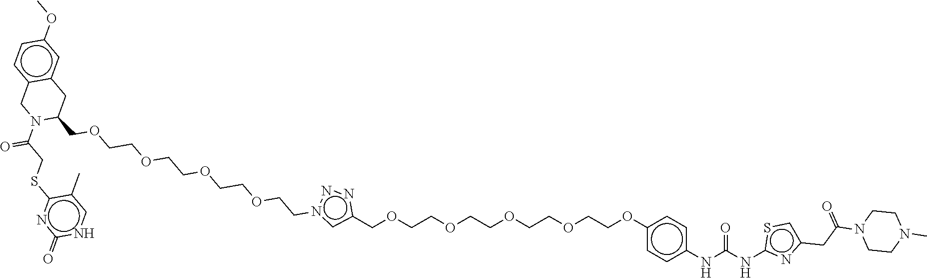 | I-497 |
| 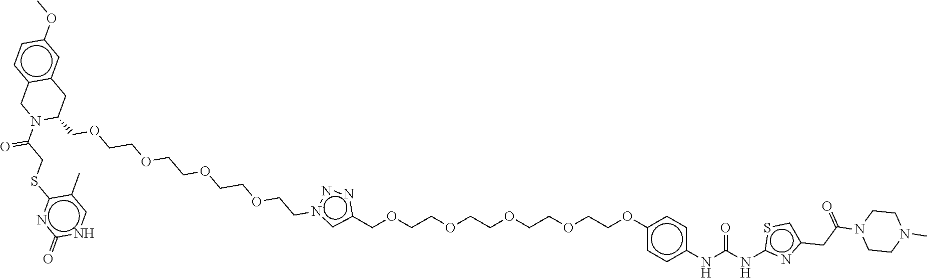 | I-498 |
| 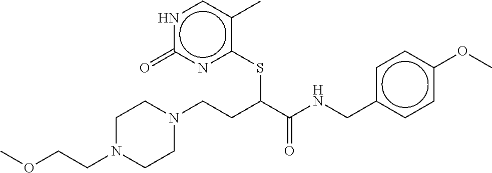 | I-499 |
| 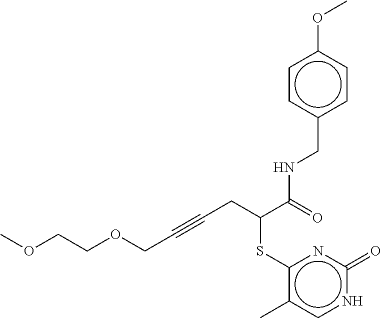 | I-500 |
| 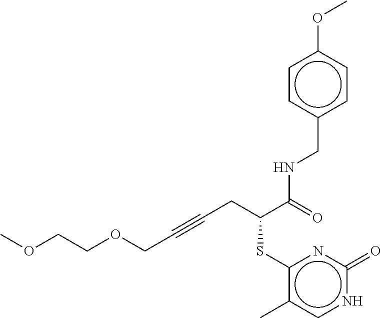 | I-501 |
| 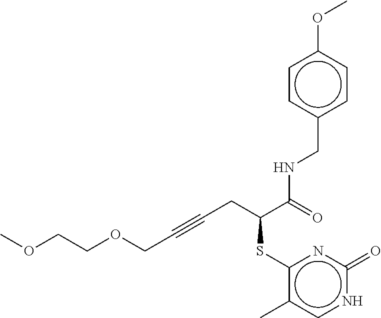 | I-502 |
| 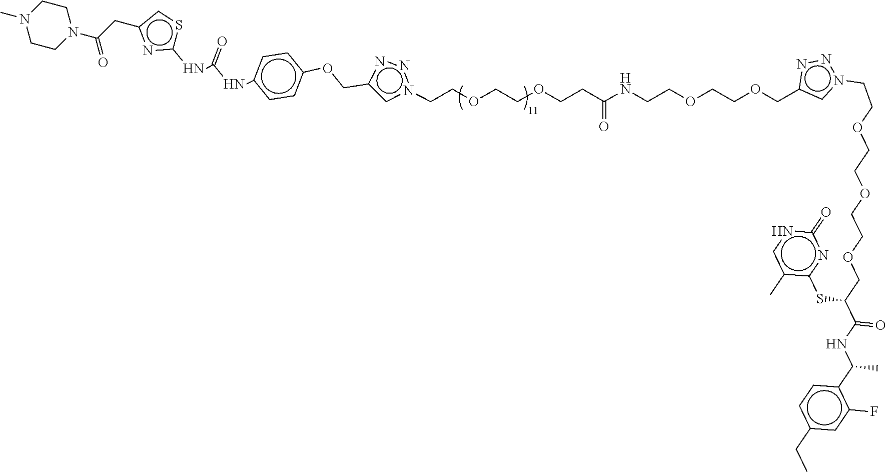 | I-503 |
| 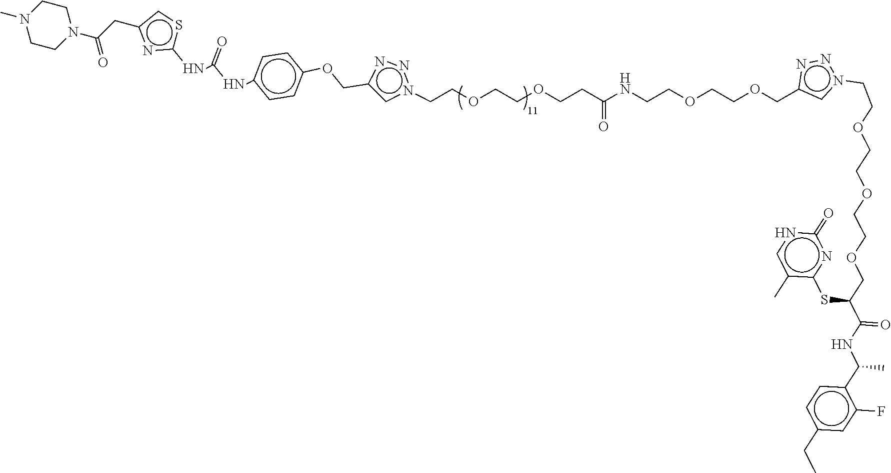 | I-504 |
| 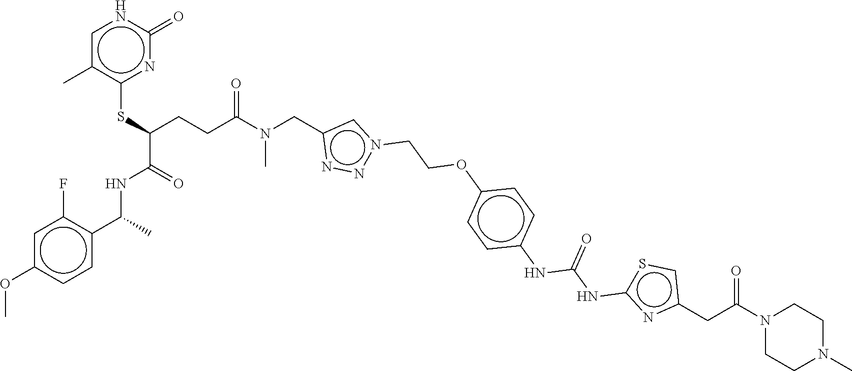 | I-505 |
| 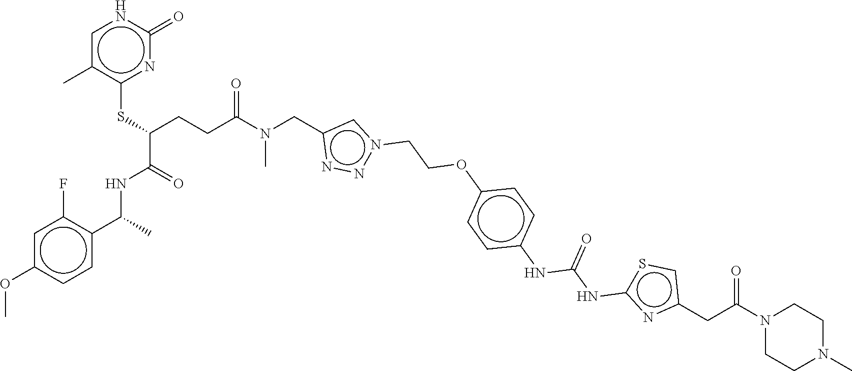 | I-506 |
| 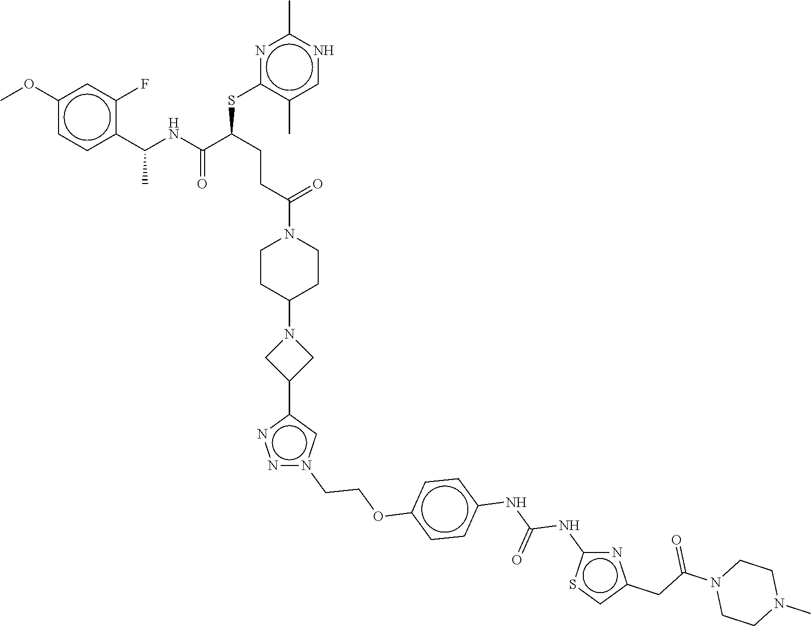 | I-507 |
| 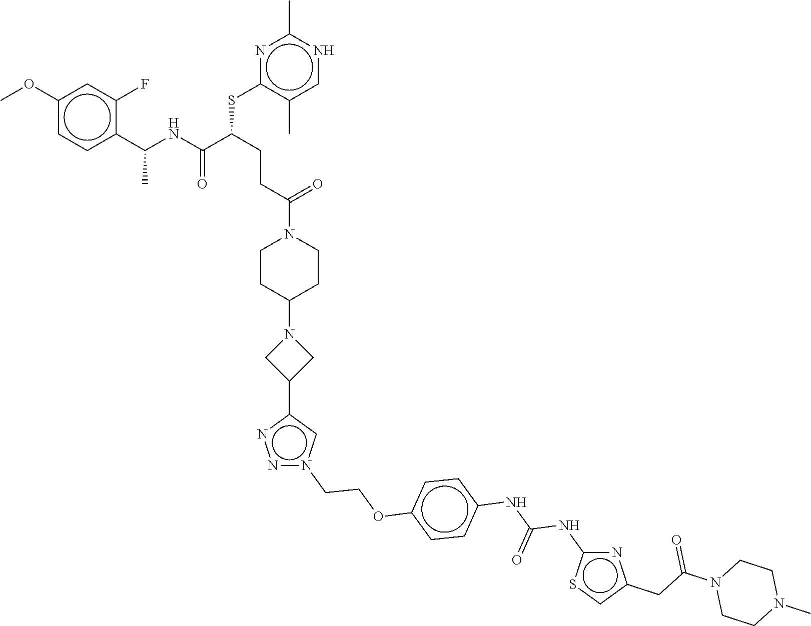 | I-508 |
| 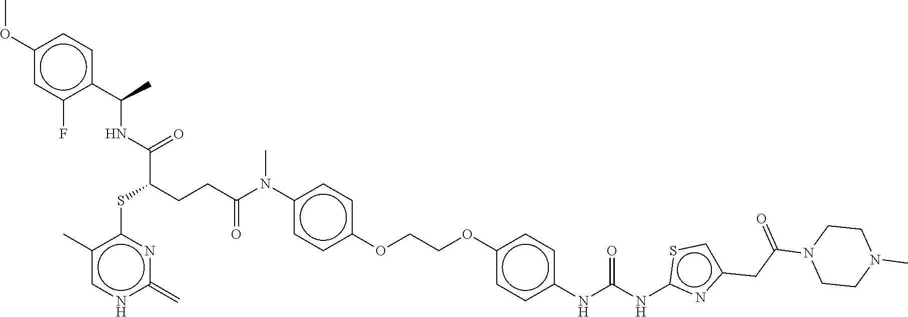 | I-509 |
| 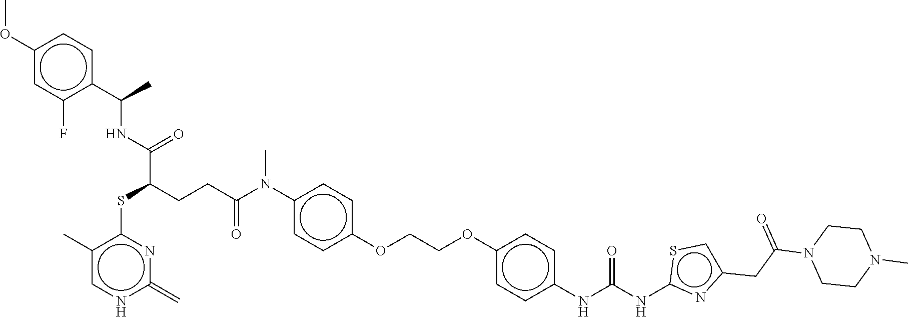 | I-510 |
| 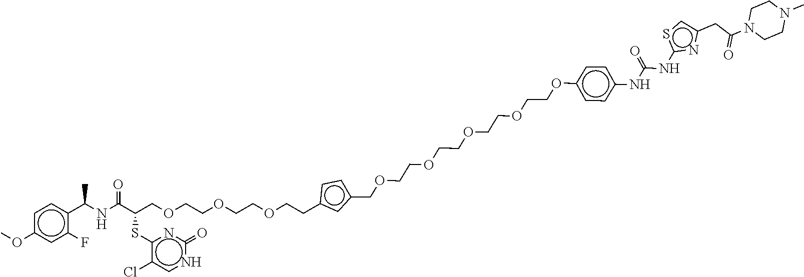 | I-511 |
| 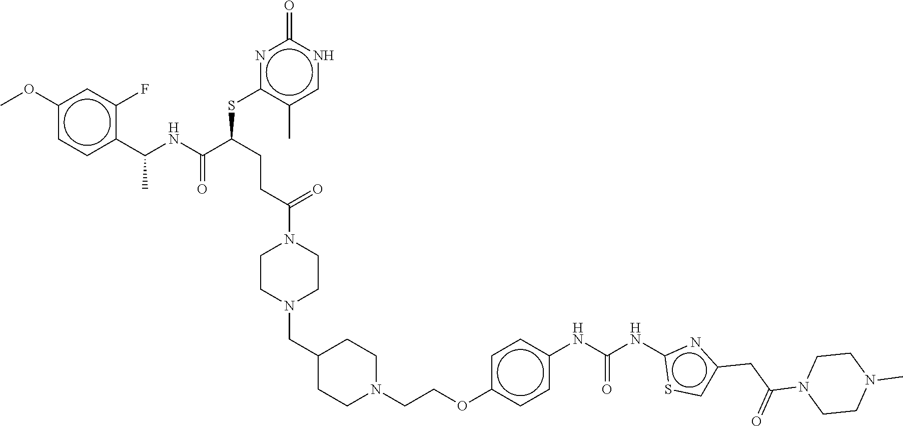 | I-512 |
| 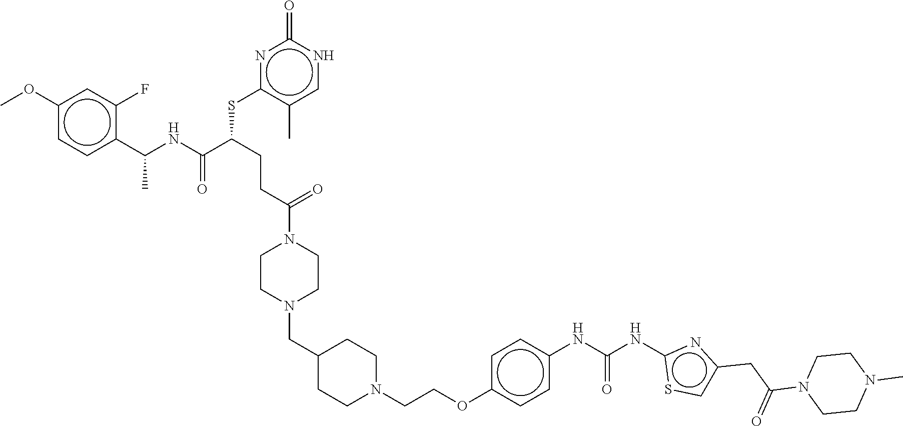 | I-513 |
| 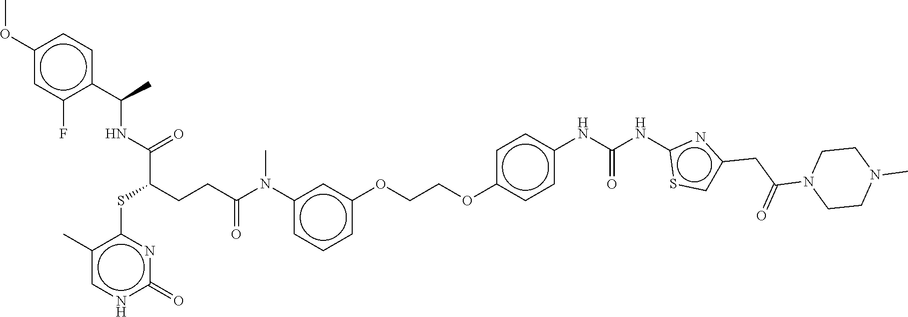 | I-514 |
| 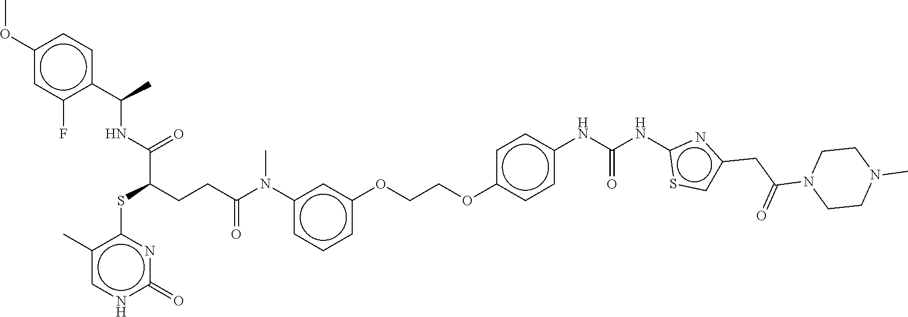 | I-515 |
| 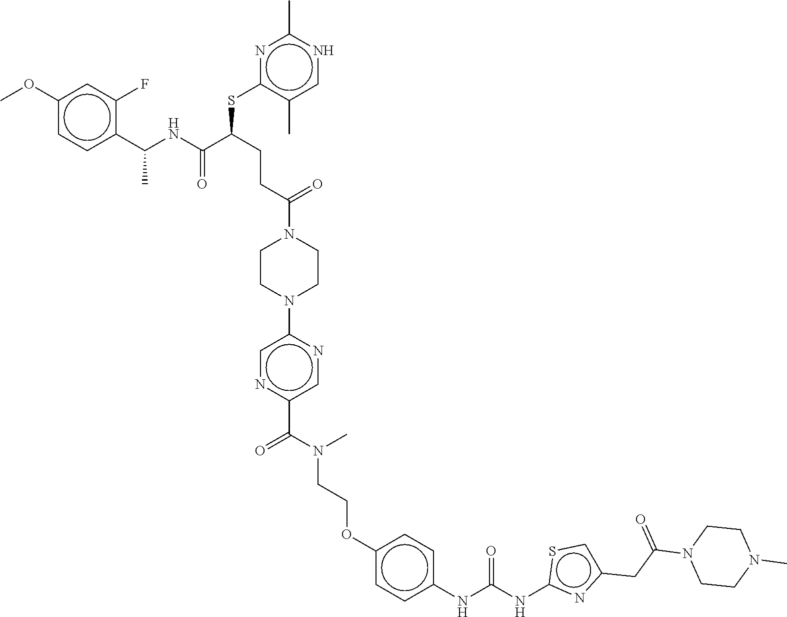 | I-516 |
| 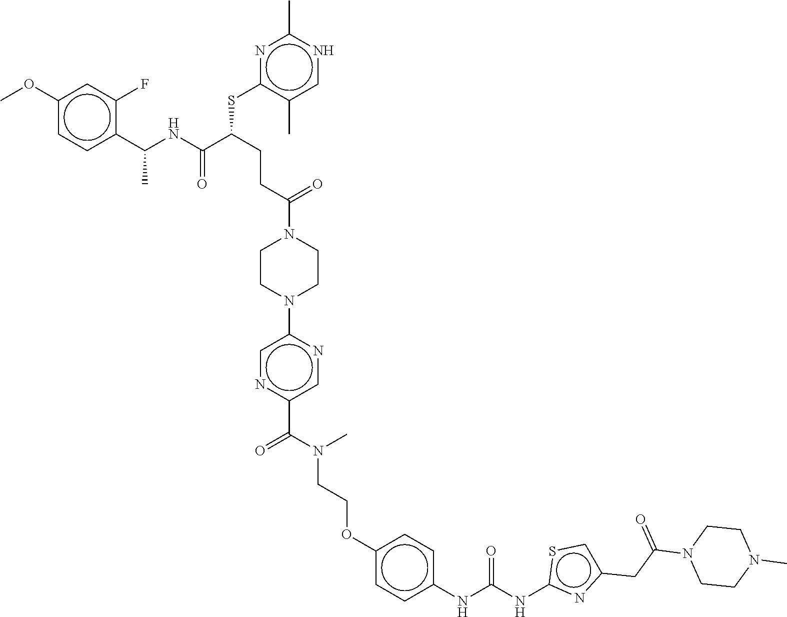 | I-517 |
| 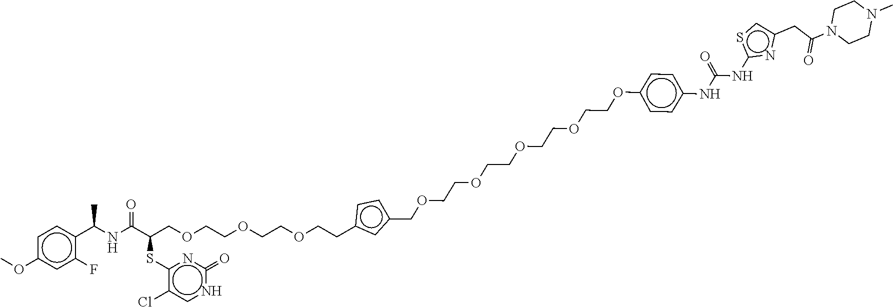 | I-518 |
| 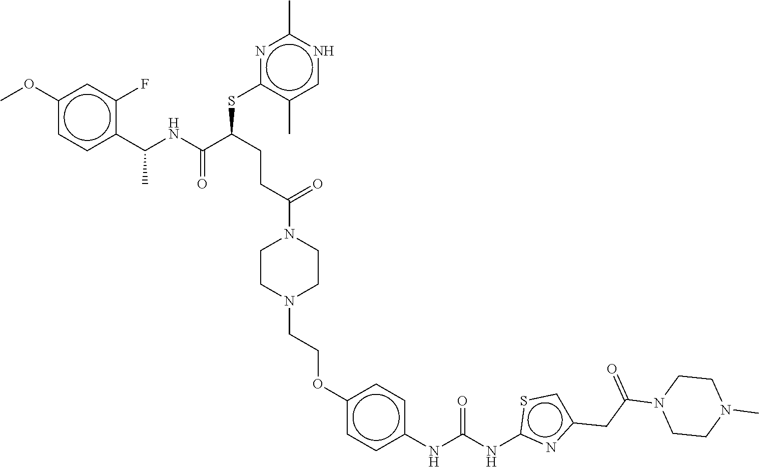 | I-519 |
| 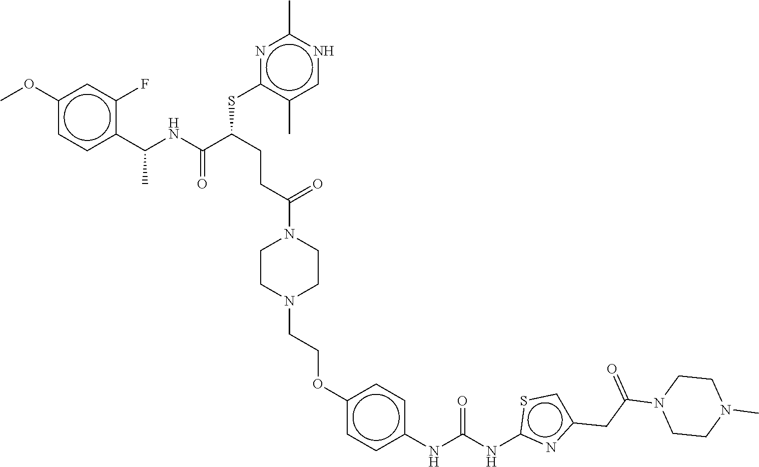 | I-520 |
| 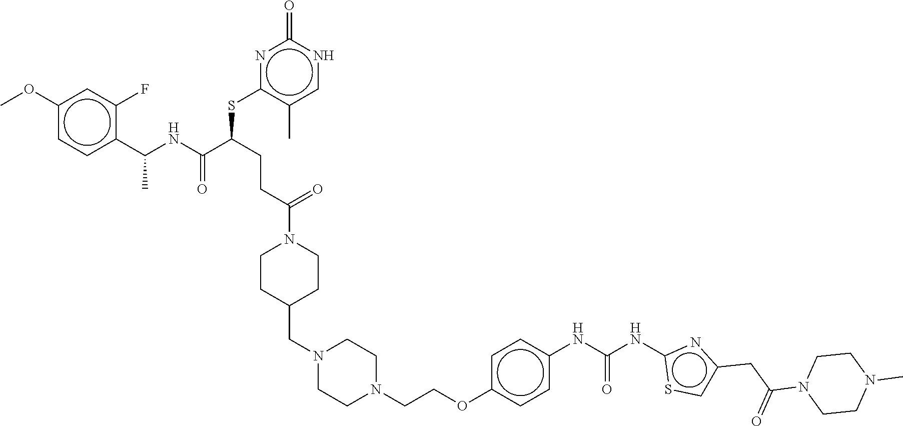 | I-521 |
| 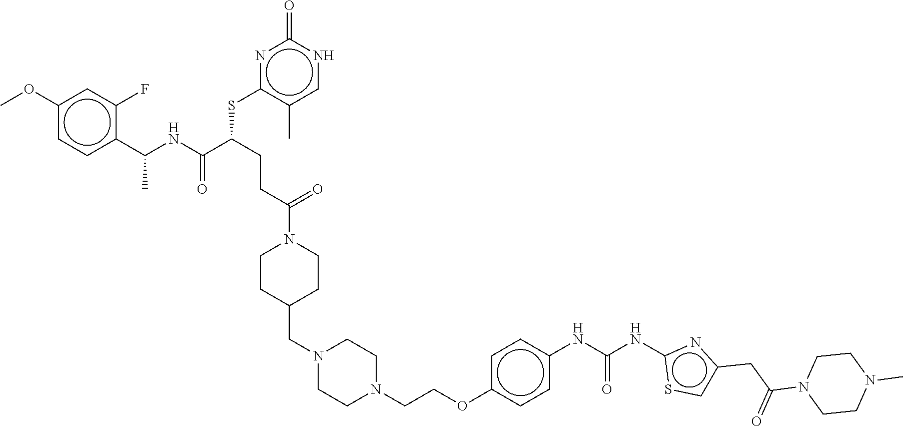 | I-522 |
| 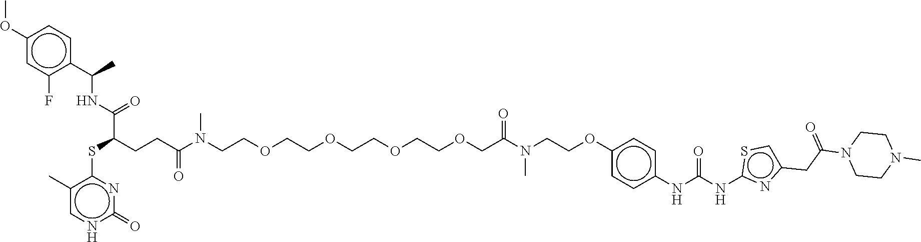 | I-523 |
| 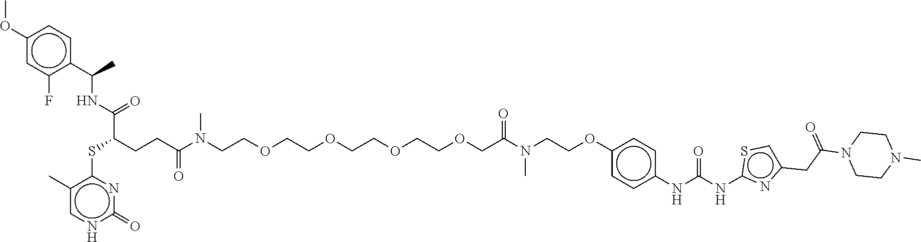 | I-524 |
| 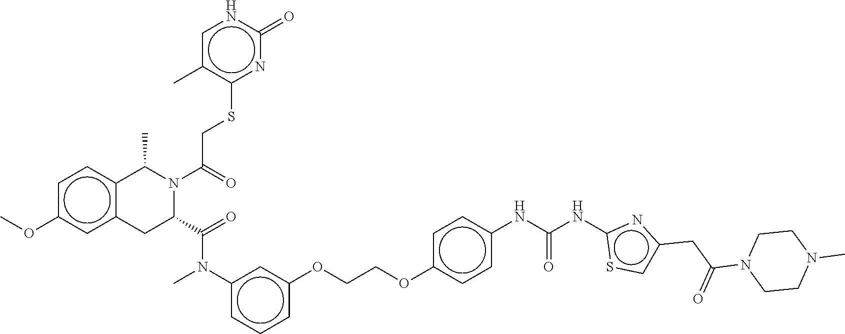 | I-525 |
| 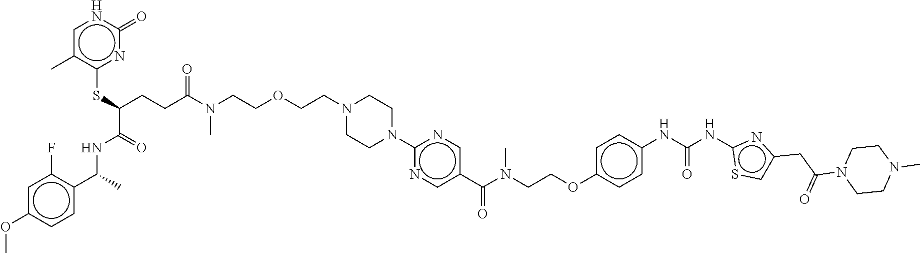 | I-526 |
| 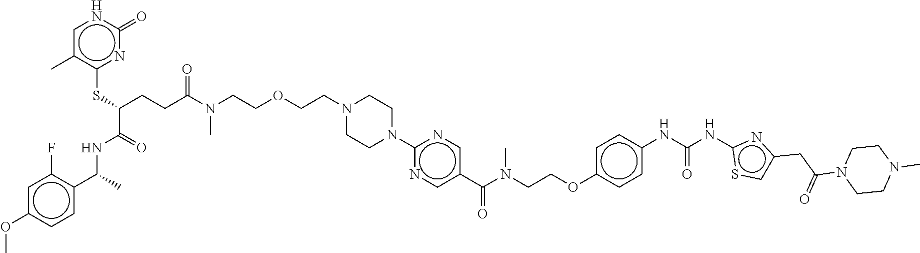 | I-527 |
| 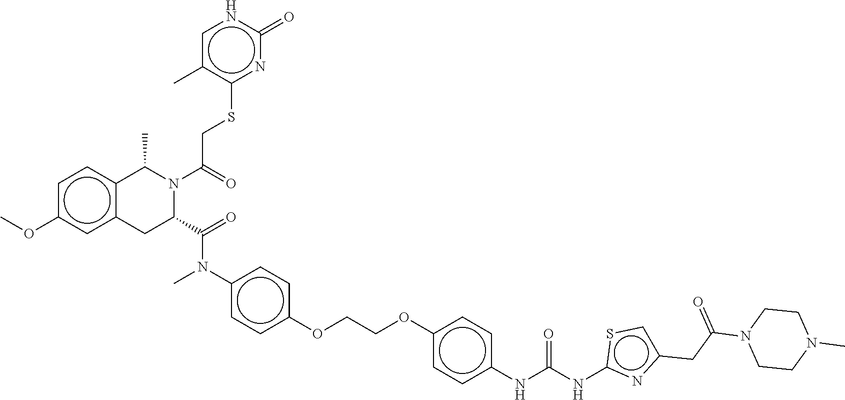 | I-528 |
| 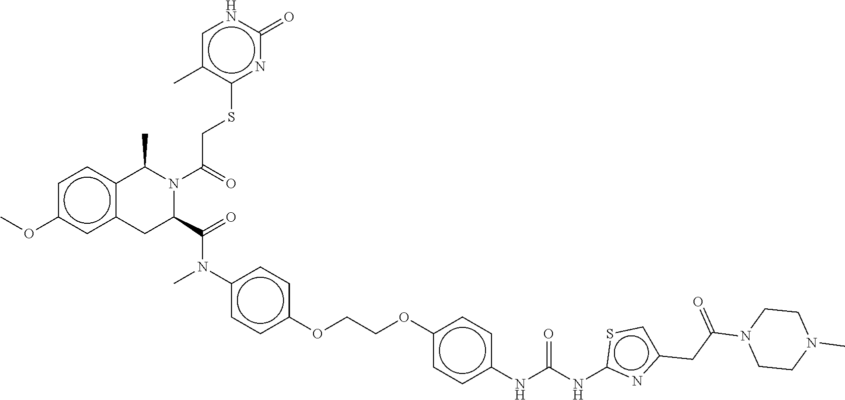 | I-529 |
| 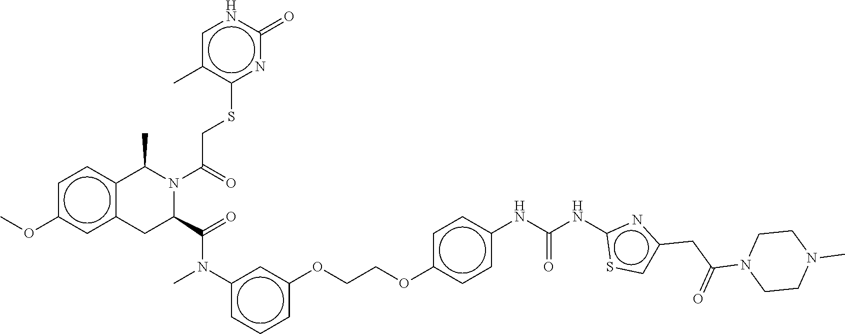 | I-530 |
| 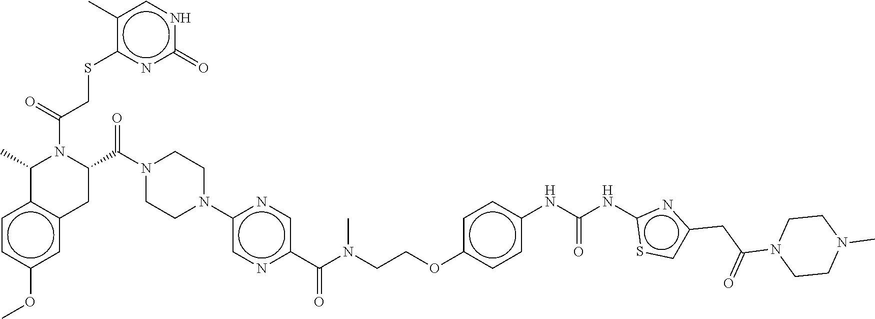 | I-531 |
| 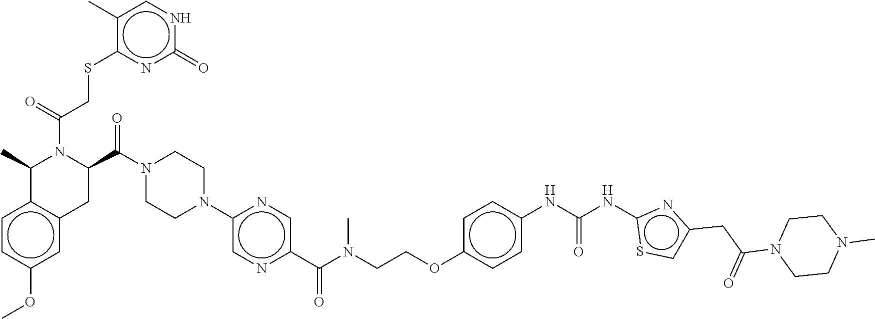 | I-532 |
| 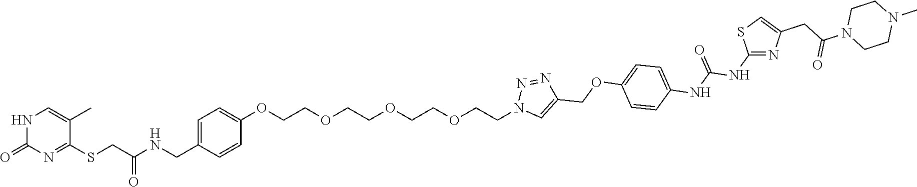 [SMILES: Cc1c[nH]c(=O)nc1SCC(=O)NCc1ccc(OCCOCCOCCOCCn2cc(COc3ccc(NC(=O)Nc4nc(CC(=O)N5CCN(C)CC5)cs4)cc3)nn2)cc1] | I-533 |
| 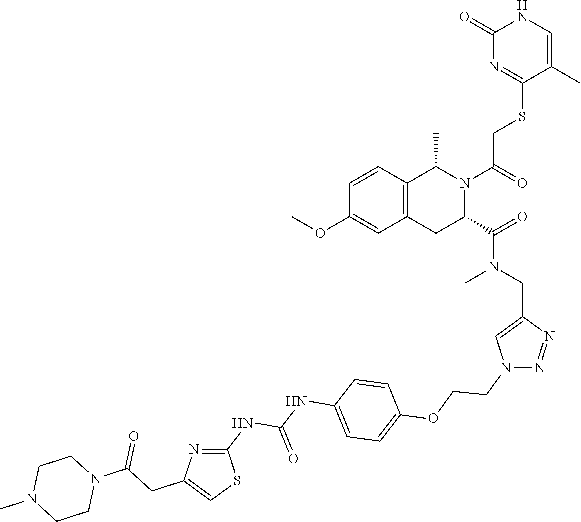 | I-534 |
| 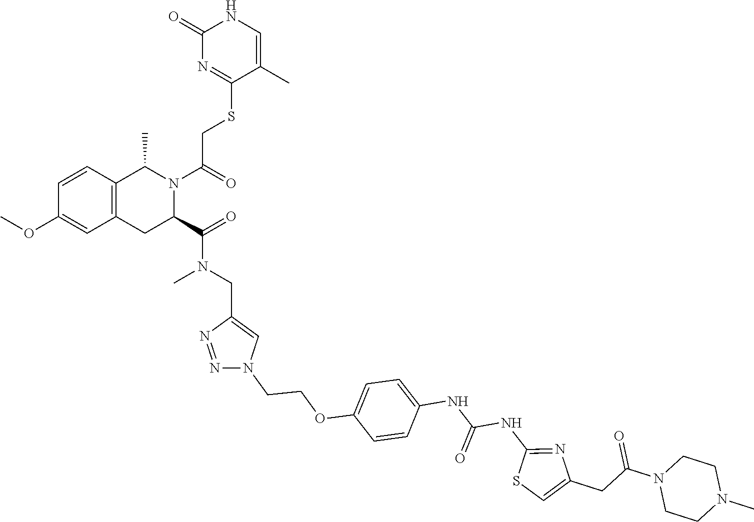 | I-535 |
| 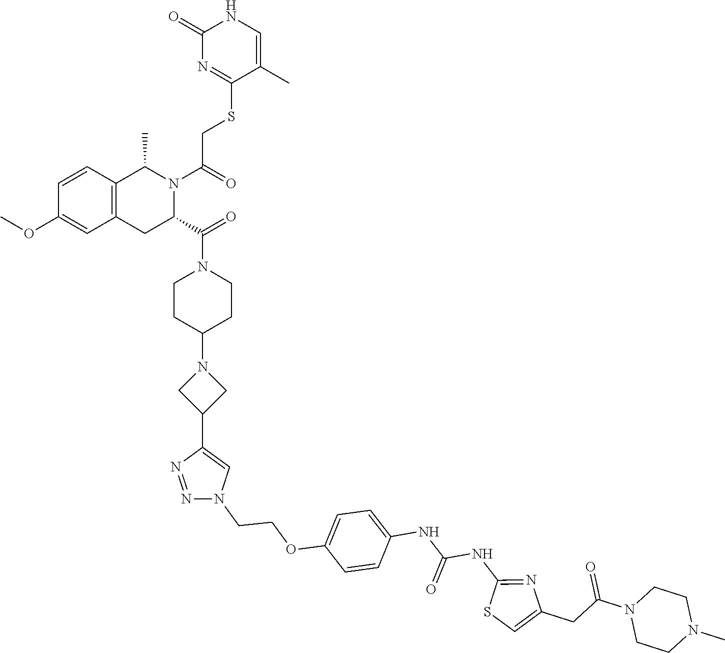 | I-536 |
| 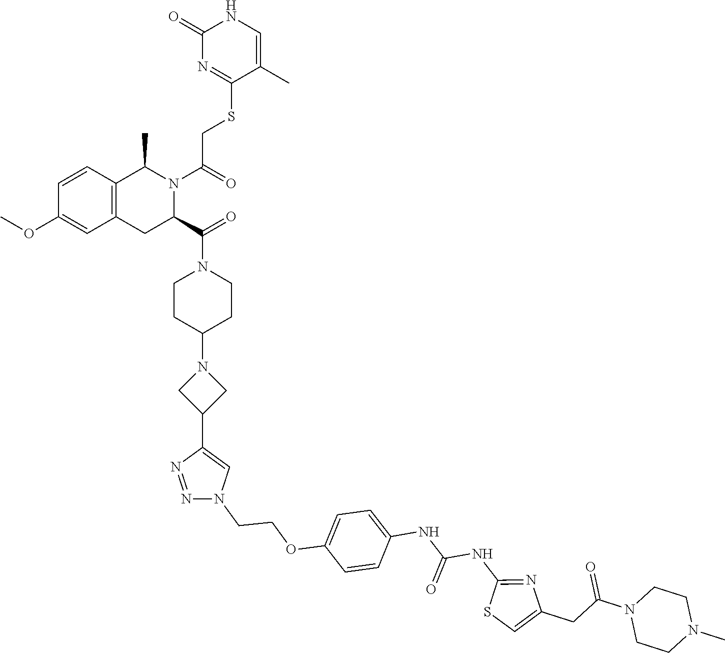 | I-537 |

In some embodiments, the compound is selected from I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, I-110, I-111, I-112, I-113, I-114, I-115, I-116, I-117, I-118, I-119, I-120, I-121, I-122, I-123, I-124, I-125, I-126, I-127, I-128, I-129, I-130, I-131, I-132, I-133, I-134, I-135, I-136, I-137, I-138, I-139, I-140, I-141, I-142, I-143, I-144, I-145, I-146, I-147, I-148, I-149, I-150, I-151, I-152, I-153, I-154, I-155, I-156, I-157, I-158, I-159, I-160, I-161, I-162 and I-163.

In some embodiments, the compound is selected from I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59 and I-60.

In some embodiments, the compound is not I-1, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, I-110, I-111, I-112, I-113, I-114, I-115, I-116, I-117, I-118, I-119, I-120, I-121, I-122, I-123, I-124, I-125, I-126, I-127, I-128, I-129, I-130, I-131, I-132, I-133, I-134, I-135, I-136, I-137, I-138, I-139, I-140, I-141, I-142, I-143, I-144, I-145, I-146, I-147, I-148, I-149, I-150, I-151, I-152, I-153, I-154, I-155, I-156, I-157, I-158, I-159, I-160, I-161, I-162 and I-163.

In some embodiments, the compound is selected from I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, I-110, I-111, I-112, I-113, I-114, I-115, I-116, I-117, I-118, I-119, I-120, I-121, I-122, I-123, I-124, I-125, I-126, I-127, I-128, I-129, I-130, 131, I-132, I-133, I-134, I-135, I-136, I-137, I-138, I-139, I-140, I-141, I-142, 143, I-144, I-145, I-146, 147, I-148, I-149, I-150, 151, I-152, I-153, I-154, 155, I-156, I-157, 158, I-159, 160, 161, 162, 163, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, 189, I-190, I-191, I-192, I-193, I-194, I-195, I-196, I-197, I-198, I-199, I-200, 201, I-202, I-203, I-204, 205, I-206, I-207, I-208, 209, I-210, I-211, I-212, 213, I-214, I-215, I-216, I-217, 218, I-219, I-220, I-221, I-222, I-223, I-224, I-225, I-226, I-227, I-228, I-229, 230, I-231, I-232, I-233, 234, I-235, I-236, I-237, 238, I-239, I-240, I-241, 242, I-243, I-244, 245, 246, 247, 248, 249, 250, 251, 252, 253, 254, 255, 256, 257, 258, 259, I-260, I-261, I-262, 263, I-264, I-265, I-266, 267, I-268, I-269, I-270, 271, I-272, I-273, I-274, I-275, 276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, 305, I-306, I-307, I-308, I-309, I-310, I-311, I-312, I-313, I-314, I-315, I-316, I-317, 318, 319, 320, and I-321.

In some embodiments, the compound is selected from I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, 27, 28, 29, I-30, I-31, I-32, I-33, I-34, I-35, I-36, I-37, I-38, I-39, I-40, I-41, I-42, I-43, I-44, I-45, 46, 47, 48, I-49, I-50, I-51, I-52, I-53, I-54, I-55, I-56, I-57, I-58, I-59, I-60, I-164, I-165, 166, I-167, I-168, I-169, 170, I-171, I-172, I-173, 174, I-175, I-176, I-177, 178, I-179, I-180, I-181, I-182, I-183, I-184, I-185, I-186, I-187, I-188, I-189, I-190, I-191, I-192, I-193, I-194, 195, I-196, I-197, I-198, 199, I-200, I-201, I-202, 203, I-204, I-205, I-206, 207, I-208, I-209, I-210, I-211, 212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, 241, I-242, I-243, I-244, I-245, I-246, I-247, I-248, I-249, I-250, I-251, I-252, 253, I-254, I-255, I-256, 257, I-258, I-259, I-260, 261, I-262, I-263, I-264, 265, I-266, I-267, I-268, I-269, 270, I-271, I-272, I-273, I-274, I-275, I-276, I-277, I-278, I-279, I-280, I-281, 282, I-283, I-284, I-285, 286, I-287, I-288, I-289, 290, I-291, I-292, I-293, 294, I-295, I-296, 297, 298, 299, 300, 301, 302, 303, 304, 305, 306, 307, 308, 309, 310, 311, 312, 313, 314, 315, 316, 317, 318, 319, I-320, and I-321.

In some embodiments, the compound is selected from 164, 165, 166, 167, 168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, I-191, I-192, I-193, I-194, I-195, I-196, I-197, I-198, I-199, I-200, I-201, I-202, I-203, I-204, I-205, I-206, I-207, I-208, I-209, I-210, I-211, I-212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, I-241, I-242, I-243, I-244, I-245, I-246, I-247, I-248, I-249, I-250, I-251, I-252, I-253, I-254, I-255, I-256, I-257, I-258, I-259, I-260, I-261, I-262, I-263, I-264, I-265, I-266, I-267, I-268, I-269, I-270, I-271, I-272, I-273, I-274, I-275, I-276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, I-305, I-306, I-307, I-308, I-309, I-310, I-311, I-312, I-313, I-314, I-315, I-316, I-317, I-318, I-319, I-320, and I-321.

In some embodiments, the compound is selected from I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, I-110, I-111, I-112, I-113, I-114, I-115, I-116, I-117, I-118, I-119, I-120, I-121, I-122, I-123, I-124, I-125, I-126, I-127, I-128, I-129, I-130, I-131, I-132, I-133, I-134, I-135, I-136, I-137, I-138, I-139, I-140, I-141, I-142, I-143, I-144, I-145, I-146, I-147, I-148, I-149, I-150, I-151, I-152, I-153, I-154, I-155, I-156, I-157, I-158, I-159, I-160, I-161, I-162, I-163, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, I-191, I-192, I-193, I-194, I-195, I-196, I-197, I-198, I-199, I-200, I-201, I-202, I-203, I-204, I-205, I-206, I-207, I-208, I-209, I-210, I-211, I-212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, I-241, I-242, I-243, I-244, I-245, I-246, I-247, I-248, I-249, I-250, I-251, I-252, I-253, I-254, I-255, I-256, I-257, I-258, I-259, I-260, I-261, I-262, I-263, I-264, I-265, I-266, I-267, I-268, I-269, I-270, I-271, I-272, I-273, I-274, I-275, I-276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, I-305, I-306, I-307, I-308, I-309, I-310, I-311, I-312, I-313, I-314, I-315, I-316, I-317, I-318, I-319, I-320, I-321, I-322, I-323, I-324, I-325, I-326, I-327, I-328, I-329, I-330, I-331, I-332, I-333, I-334, I-335, I-336, I-337, I-338, I-339, I-340, I-341, I-342, I-343, I-344, I-345, I-346, I-347, I-348, I-349, I-350, I-351, I-352, I-353, I-354, I-355, I-356, I-357, I-358, I-359, I-360, I-361, I-362, I-363, I-364, I-365, I-366, I-367, I-368, I-369, I-370, I-371, I-372, I-373, I-374, I-375, I-376, I-377, I-378, I-379, I-380, I-381, I-382, I-383, I-384, I-385, I-386, I-387, I-388, I-389, I-390, I-391, I-392, I-393, I-394, I-395, I-396, I-397, I-398, I-399, I-400, I-401, I-402, I-403, I-404, I-405, I-406, I-407, I-408, I-409, I-410, I-411, I-412, I-413, I-414, I-415, I-416, I-417, I-418, I-419, I-420, I-421, I-422, I-423, I-424, I-425, I-426, I-427, I-428, I-429, I-430, I-431, I-432, I-433, I-434, I-435, I-436, I-437, I-438, I-439, I-440, I-441, and I-442.

In some embodiments, the compound is selected from I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, I-191, I-192, I-193, I-194, I-195, I-196, I-197, I-198, I-199, I-200, I-201, I-202, I-203, I-204, I-205, I-206, I-207, I-208, I-209, I-210, I-211, I-212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, I-241, I-242, I-243, I-244, I-245, I-246, I-247, I-248, I-249, I-250, I-251, I-252, I-253, I-254, I-255, I-256, I-257, I-258, I-259, I-260, I-261, I-262, I-263, I-264, I-265, I-266, I-267, I-268, I-269, I-270, I-271, I-272, I-273, I-274, I-275, I-276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, I-305, I-306, I-307, I-308, I-309, I-310, I-311, I-312, I-313, I-314, I-315, I-316, I-317, I-318, I-319, I-320, I-321, I-322, I-323, I-324, I-325, I-326, I-327, I-328, I-329, I-330, I-331, I-332, I-333, I-334, I-335, I-336, I-337, I-338, I-339, I-340, I-341, I-342, I-343, I-344, I-345, I-346, I-347, I-348, I-349, I-350, I-351, I-352, I-353, I-354, I-355, I-356, I-357, I-358, I-359, I-360, I-361, I-362, I-363, I-364, I-365, I-366, I-367, I-368, I-369, I-370, I-371, I-372, I-373, I-374, I-375, I-376, I-377, I-378, I-379, I-380, I-381, I-382, I-383, I-384, I-385, I-386, I-387, I-388, I-389, I-390, I-391, I-392, I-393, I-394, I-395, I-396, I-397, I-398, I-399, I-400, I-401, I-402, I-403, I-404, I-405, I-406, I-407, I-408, I-409, I-410, I-411, I-412, I-413, I-414, I-415, I-416, I-417, I-418, I-419, I-420, I-421, I-422, I-423, I-424, I-425, I-426, I-427, I-428, I-429, I-430, I-431, I-432, I-433, I-434, I-435, I-436, I-437, I-438, I-439, I-440, I-441, and I-442.

In some embodiments, the compound is selected from I-322, I-323, I-324, I-325, I-326, I-327, I-328, I-329, I-330, I-331, I-332, I-333, I-334, I-335, I-336, I-337, I-338, I-339, I-340, I-341, I-342, I-343, I-344, I-345, I-346, I-347, I-348, I-349, I-350, I-351, I-352, I-353, I-354, I-355, I-356, I-357, I-358, I-359, I-360, I-361, I-362, I-363, I-364, I-365, I-366, I-367, I-368, I-369, I-370, I-371, I-372, I-373, I-374, I-375, I-376, I-377, I-378, I-379, I-380, I-381, I-382, I-383, I-384, I-385, I-386, I-387, I-388, I-389, I-390, I-391, I-392, I-393, I-394, I-395, I-396, I-397, I-398, I-399, I-400, I-401, I-402, I-403, I-404, I-405, I-406, I-407, I-408, I-409, I-410, I-411, I-412, I-413, I-414, I-415, I-416, I-417, I-418, I-419, I-420, I-421, I-422, I-423, I-424, I-425, I-426, I-427, I-428, I-429, I-430, I-431, I-432, I-433, I-434, I-435, I-436, I-437, I-438, I-439, I-440, I-441, and I-442.

In some embodiments, the compound is selected from I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, I-110, I-111, I-112, I-113, I-114, I-115, I-116, I-117, I-118, I-119, I-120, I-121, I-122, I-123, I-124, I-125, I-126, I-127, I-128, I-129, I-130, I-131, I-132, I-133, I-134, I-135, I-136, I-137, I-138, I-139, I-140, I-141, I-142, I-143, I-144, I-145, I-146, I-147, I-148, I-149, I-150, I-151, I-152, I-153, I-154, I-155, I-156, I-157, I-158, I-159, I-160, I-161, I-162, I-163, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, I-191, I-192, I-193, I-194, I-195, I-196, I-197, I-198, I-199, I-200, I-201, I-202, I-203, I-204, I-205, I-206, I-207, I-208, I-209, I-210, I-211, I-212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, I-241, I-242, I-243, I-244, I-245, I-246, I-247, I-248, I-249, I-250, I-251, I-252, I-253, I-254, I-255, I-256, I-257, I-258, I-259, I-260, I-261, I-262, I-263, I-264, I-265, I-266, I-267, I-268, I-269, I-270, I-271, I-272, I-273, I-274, I-275, I-276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, I-305, I-306, I-307, I-308, I-309, I-310, I-311, I-312, I-313, I-314, I-315, I-316, I-317, I-318, I-319, I-320, I-321, I-322, I-323, I-324, I-325, I-326, I-327, I-328, I-329, I-330, I-331, I-332, I-333, I-334, I-335, I-336, I-337, I-338, I-339, I-340, I-341, I-342, I-343, I-344, I-345, I-346, I-347, I-348, I-349, I-350, I-351, I-352, I-353, I-354, I-355, I-356, I-357, I-358, I-359, I-360, I-361, I-362, I-363, I-364, I-365, I-366, I-367, I-368, I-369, I-370, I-371, I-372, I-373, I-374, I-375, I-376, I-377, I-378, I-379, I-380, I-381, I-382, I-383, I-384, I-385, I-386, I-387, I-388, I-389, I-390, I-391, I-392, I-393, I-394, I-395, I-396, I-397, I-398, I-399, I-400, I-401, I-402, I-403, I-404, I-405, I-406, I-407, I-408, I-409, I-410, I-411, I-412, I-413, I-414, I-415, I-416, I-417, I-418, I-419, I-420, I-421, I-422, I-423, I-424, I-425, I-426, I-427, I-428, I-429, I-430, I-431, I-432, I-433, I-434, I-435, I-436, I-437, I-438, I-439, I-440, I-441, I-442, I-443, I-444, I-445, I-446, I-447, I-448, I-449, I-450, I-451, I-452, I-453, I-454, I-455, I-456, I-457, I-458, I-459, I-460, I-461, I-462, I-463, I-464, I-465, I-466, I-467, I-468, I-469, I-470, I-471, I-472, I-473, I-474, I-475, I-476, I-477, I-478, I-479, I-480, I-481, I-482, I-483, I-484, I-485, I-486, I-487, I-488, I-489, I-490, I-491, I-492, I-493, I-494, I-495, I-496, I-497, I-498, I-499, I-500, I-501, I-502, I-503, I-504, I-505, I-506, I-507, I-508, I-509, I-510, I-511, I-512, I-513, I-514, I-515, I-516, I-517, I-518, I-519, I-520, I-521, I-522, I-523, I-524, I-525, I-526, I-527, I-528, I-529, I-530, I-531, I-532, and I-533.

In some embodiments, the compound is selected from I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, I-191, I-192, I-193, I-194, I-195, I-196, I-197, I-198, I-199, I-200, I-201, I-202, I-203, I-204, I-205, I-206, I-207, I-208, I-209, I-210, I-211, I-212, I-213, I-214, I-215, I-216, I-217, I-218, I-219, I-220, I-221, I-222, I-223, I-224, I-225, I-226, I-227, I-228, I-229, I-230, I-231, I-232, I-233, I-234, I-235, I-236, I-237, I-238, I-239, I-240, I-241, I-242, I-243, I-244, I-245, I-246, I-247, I-248, I-249, I-250, I-251, I-252, I-253, I-254, I-255, I-256, I-257, I-258, I-259, I-260, I-261, I-262, I-263, I-264, I-265, I-266, I-267, I-268, I-269, I-270, I-271, I-272, I-273, I-274, I-275, I-276, I-277, I-278, I-279, I-280, I-281, I-282, I-283, I-284, I-285, I-286, I-287, I-288, I-289, I-290, I-291, I-292, I-293, I-294, I-295, I-296, I-297, I-298, I-299, I-300, I-301, I-302, I-303, I-304, I-305, I-306, I-307, I-308, I-309, I-310, I-311, I-312, I-313, I-314, I-315, I-316, I-317, I-318, I-319, I-320, I-321, I-322, I-323, I-324, I-325, I-326, I-327, I-328, I-329, I-330, I-331, I-332, I-333, I-334, I-335, I-336, I-337, I-338, I-339, I-340, I-341, I-342, I-343, I-344, I-345, I-346, I-347, I-348, I-349, I-350, I-351, I-352, I-353, I-354, I-355, I-356, I-357, I-358, I-359, I-360, I-361, I-362, I-363, I-364, I-365, I-366, I-367, I-368, I-369, I-370, I-371, I-372, I-373, I-374, I-375, I-376, I-377, I-378, I-379, I-380, I-381, I-382, I-383, I-384, I-385, I-386, I-387, I-388, I-389, I-390, I-391, I-392, I-393, I-394, I-395, I-396, I-397, I-398, I-399, I-400, I-401, I-402, I-403, I-404, I-405, I-406, I-407, I-408, I-409, I-410, I-411, I-412, I-413, I-414, I-415, I-416, I-417, I-418, I-419, I-420, I-421, I-422, I-423, I-424, I-425, I-426, I-427, I-428, I-429, I-430, I-431, I-432, I-433, I-434, I-435, I-436, I-437, I-438, I-439, I-440, I-441, I-442, I-443, I-444, I-445, I-446, I-447, I-448, I-449, I-450, I-451, I-452, I-453, I-454, I-455, I-456, I-457, I-458, I-459, I-460, I-461, I-462, I-463, I-464, I-465, I-466, I-467, I-468, I-469, I-470, I-471, I-472, I-473, I-474, I-475, I-476, I-477, I-478, I-479, I-480, I-481, I-482, I-483, I-484, I-485, I-486, I-487, I-488, I-489, I-490, I-491, I-492, I-493, I-494, I-495, I-496, I-497, I-498, I-499, I-500, I-501, I-502, I-503, I-504, I-505, I-506, I-507, I-508, I-509, I-510, I-511, I-512, I-513, I-514, I-515, I-516, I-517, I-518, I-519, I-520, I-521, I-522, I-523, I-524, I-525, I-526, I-527, I-528, I-529, I-530, I-531, I-532, and I-533.

In some embodiments, the compound is selected from I-443, I-444, I-445, I-446, I-447, I-448, I-449, I-450, I-451, I-452, I-453, I-454, I-455, I-456, I-457, I-458, I-459, I-460, I-461, I-462, I-463, I-464, I-465, I-466, I-467, I-468, I-469, I-470, I-471, I-472, I-473, I-474, I-475, I-476, I-477, I-478, I-479, I-480, I-481, I-482, I-483, I-484, I-485, I-486, I-487, I-488, I-489, I-490, I-491, I-492, I-493, I-494, I-495, I-496, I-497, I-498, I-499, I-500, I-501, I-502, I-503, I-504, I-505, I-506, I-507, I-508, I-509, I-510, I-511, I-512, I-513, I-514, I-515, I-516, I-517, I-518, I-519, I-520, I-521, I-522, I-523, I-524, I-525, I-526, I-527, I-528, I-529, I-530, I-531, I-532, and I-533.

In some embodiments, the compound is selected from I-534, I-535, I-536, and I-537.

In some embodiments, the compound is selected from one of those shown in Table 1, above. In some embodiments, the compound is selected from one of those shown in Table 1, above, or a pharmaceutically acceptable salt thereof. In some embodiments, the compound is selected from one of those shown in Table 1A, above. In some embodiments, the compound is selected from one of those shown in Table 1A, above, or a pharmaceutically acceptable salt thereof. In some embodiments, the compound is selected from one of those shown in Table 1B, above. In some embodiments, the compound is selected from one of those shown in Table 1B, above, or a pharmaceutically acceptable salt thereof. In some embodiments, the compound is selected from one of those shown in Table 1C, above. In some embodiments, the compound is selected from one of those shown in Table 1C, above, or a pharmaceutically acceptable salt thereof.

In some embodiments, the compounds of the present invention can be used as CNOT7 binders. In some embodiments, the compounds of the present invention can be used to modulate the activity of CNOT7. In some embodiments, the compounds of the present invention can be used to inhibit the activity of CNOT7. Inhibition and/or degradation of CNOT7 has been shown to suppress metastases and cell proliferation, which is beneficial, for instance, in the treatment of cancer, see e.g., Ren et al. FEBS Open Bio 10, 2020, 847-860, or Faraji et al. PLOS Genetics 12(1)e1005820, 2016.

In one aspect, the present invention provides a bifunctional compound comprising one of the CNOT7 binders described herein conjugated to a ubiquitin binding moiety capable of binding to a ubiquitin ligase such as an E3 Ubiquitin Ligase. Examples of E3 Ubiquitin Ligases include cereblon. In some embodiments, the bifunctional compound is of Formula C:

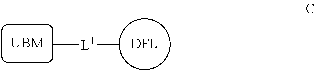

or a pharmaceutically acceptable salt thereof, wherein:
UBM is a ubiquitin binding moiety capable of binding to a ubiquitin ligase;
DFL is a Decay Factor-recruiting Ligand; and
-L¹- is a bivalent linker group that covalently connects the UBM to the DFL;
wherein the DFL binds to CNOT7. Exemplary -L¹- groups include those described above in the context of Formula A or shown in Table 3. Exemplary UBM moieties include those described in WO 2019/060742, which is hereby incorporated by reference in its entirety.

### 2. Compounds and Related Definitions

As described generally above, the present invention provides a bifunctional compound of Formula A:

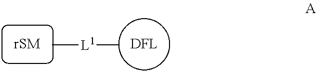

or a pharmaceutically acceptable salt thereof, wherein:
rSM is an RNA-binding small molecule that binds to a target RNA transcript;
DFL is a Decay Factor-recruiting Ligand; and
-L¹- is a bivalent linker group that covalently connects the rSM to the DFL;
wherein the DFL binds to or recruits one or more decay factors that degrade the target RNA transcript.
   RNA-Binding Small Molecules (rSMs)

In one aspect, the disclosure provides bifunctional compounds of Formula A wherein the compound includes an rSM. A variety of rSMs known in the art may be used in accordance with the present invention. In some embodiments, the rSM is modified from its known structure in order to covalently attach the rSM to the linker, L¹, at any available and modifiable C atom or a heteroatom such as an N, O, S, or P atom of the rSM. In the context of a C atom, “modifiable” refers to a C atom having 1) an attached H atom that can be replaced by a bond to L¹ via a chemical reaction such as an oxidation, reduction, nucleophilic substitution, or cross-coupling reaction; or 2) a C atom that can participate in a chemical reaction such as oxidation, reduction, nucleophilic substitution, or cross-couple reaction due to unsaturation or the presence of a leaving group attached to the C atom. For example, a C═O group, C═N group, or C—Br group is “modifiable.” Similarly, a modifiable heteroatom may be attached to an H atom capable of being replaced by a bond to L¹, or is modifiable due to unsaturation or the presence of a leaving group attached to the heteroatom.

In some embodiments, the rSM is a small molecule or pharmaceutically acceptable salt thereof. In some embodiments, the rSM has a molecular weight (MW) of 1000 or less. In some embodiments, the rSM has a MW of about 750 or less. In some embodiments, the rSM has a MW of about 600 or less. In some embodiments, the rSM has a MW of about 500 or less. In some embodiments, the rSM has a MW of between about 100 and about 1000. In some embodiments, the rSM has a MW of between about 150 and about 800, about 150 and about 600, about 150 and about 400, about 150 and about 350, about 200 and about 350, or between about 200 and about 450.

In some embodiments, the rSM or compound of Formula A binds to the target RNA transcript, or an isoform, fragment, or mutant thereof, with a K_{d} of 1 μM, 500 nM, 100 nM, 50 nM, 10 nM, 1 nM, 500 pM, 10 pM, or 1 pM or lower affinity under biological conditions. In some embodiments, the rSM or compound binds to the target RNA transcript, or an isoform, fragment, or mutant thereof, with a K_{d} of 0.1 nm to 500 nm, 10 nm to 250 nm, 0.001-25 μM, 0.01-25 μM, 0.1-25 μM, 0.1-15 μM, 0.01-10 μM, 0.001-1 μM, 0.001-0.1 μM, or 0.001-0.01 μM.

Exemplary rSMs

In some embodiments, rSM is

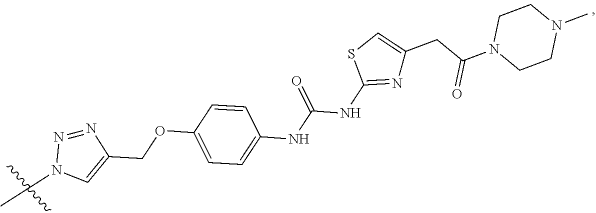 [SMILES: *n1cc(COc2ccc(NC(=O)Nc3nc(CC(=O)N4CCN(C)CC4)cs3)cc2)nn1]

wherein the rSM is covalently bound to L¹. In some embodiments, rSM is

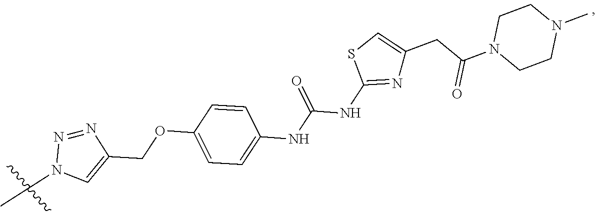 [SMILES: *n1cc(COc2ccc(NC(=O)Nc3nc(CC(=O)N4CCN(C)CC4)cs3)cc2)nn1]

wherein the rSM is covalently bound to

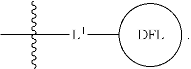

In some embodiments, rSM is

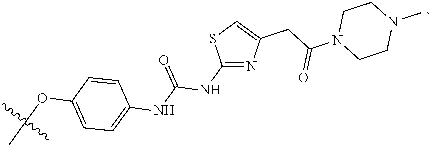 [SMILES: *Oc1ccc(NC(=O)Nc2nc(CC(=O)N3CCN(C)CC3)cs2)cc1]

wherein the rSM is covalently bound to L¹. In some embodiments, rSM is

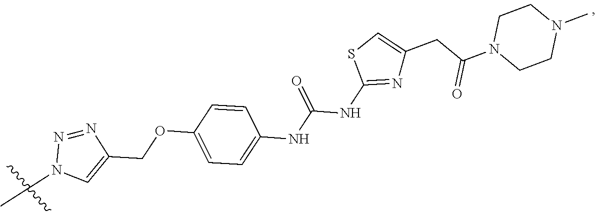 [SMILES: *n1cc(COc2ccc(NC(=O)Nc3nc(CC(=O)N4CCN(C)CC4)cs3)cc2)nn1]

wherein the rSM is covalently bound to any of the compounds of Table 1. In some embodiments, rSM is

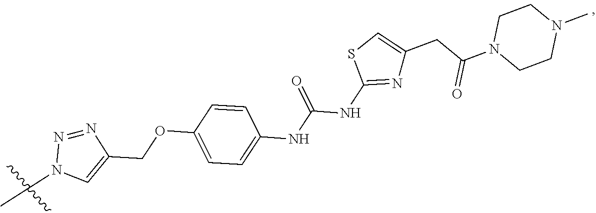 [SMILES: *n1cc(COc2ccc(NC(=O)Nc3nc(CC(=O)N4CCN(C)CC4)cs3)cc2)nn1]

wherein the rSM is covalently bound to any of the compounds of Table 1A.

In some embodiments, rSM is

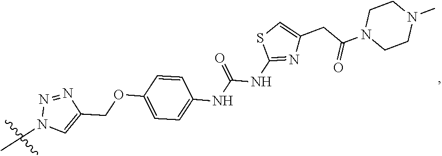 [SMILES: *n1cc(COc2ccc(NC(=O)Nc3nc(CC(=O)N4CCN(C)CC4)cs3)cc2)nn1]

wherein the rSM is covalently bound to any of the compounds of Table 1B.

In some embodiments, rSM is

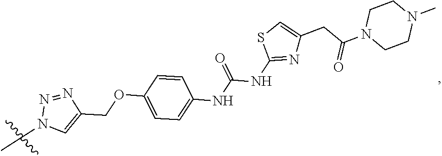 [SMILES: *n1cc(COc2ccc(NC(=O)Nc3nc(CC(=O)N4CCN(C)CC4)cs3)cc2)nn1]

wherein the rSM is covalently bound to any of the compounds of Table 1C.

In some embodiments, the rSM is selected from one of the following:

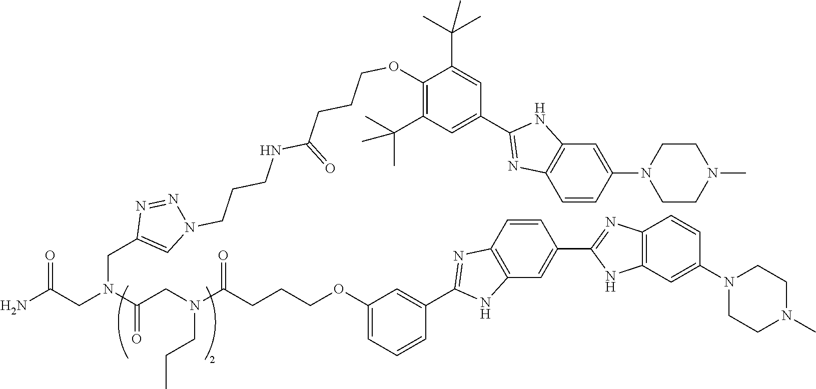

or a pharmaceutically acceptable salt thereof, wherein the rSM is covalently bound to L at any available modifiable C, N, or O atom.

In some embodiments, the rSM is a G-quadruplex binder, such as one of those described in Peng, W. et al., J. Med. Chem. 2018, 61, 6629-6646, which is hereby incorporated by reference.

In some embodiments, the rSM is a compound disclosed in Shi, Y. et al., Cell Chem. Biol. 2019, 26, 319-330, which is hereby incorporated by reference, such as one of the following:

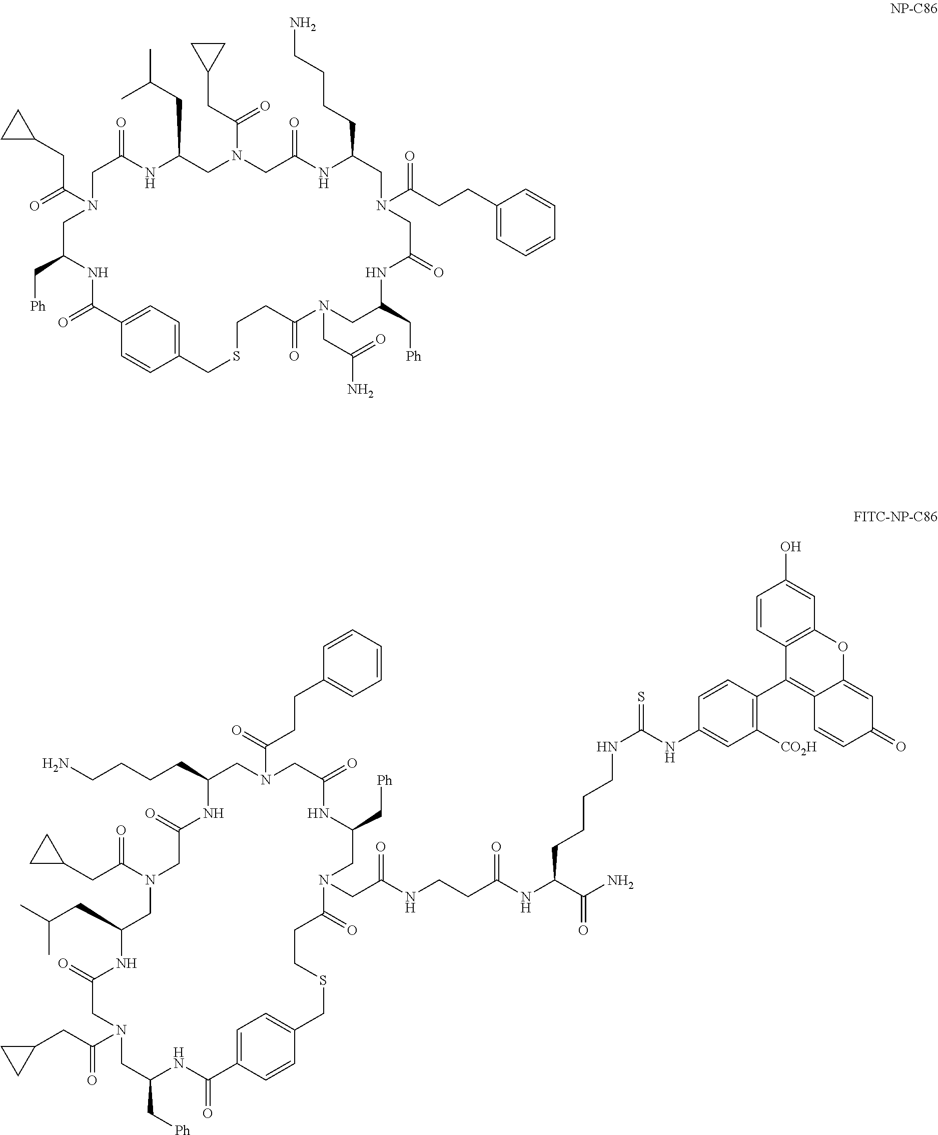

or a pharmaceutically acceptable salt thereof; wherein the rSM is covalently bound to L¹ at any available modifiable C, N, O, S, or P atom.

In some embodiments, the rSM is a compound disclosed in Velagapudi, S. P. et al. (2014), “Sequence-based design of bioactive small molecules that target precursor microRNAs,” Nat. Chem. Biol. 10, 291, hereby incorporated by reference, for example the following:

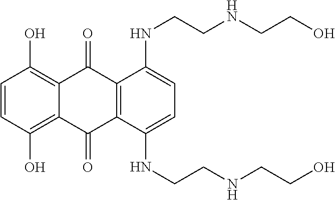 [SMILES: O=C1c2c(O)ccc(O)c2C(=O)c2c(NCCNCCO)ccc(NCCNCCO)c21]

or a pharmaceutically acceptable salt thereof, wherein the rSM is covalently bound to L¹ at any available modifiable C, N, or O atom.

In some embodiments, the rSM is a MALAT-1 binder such as the following:

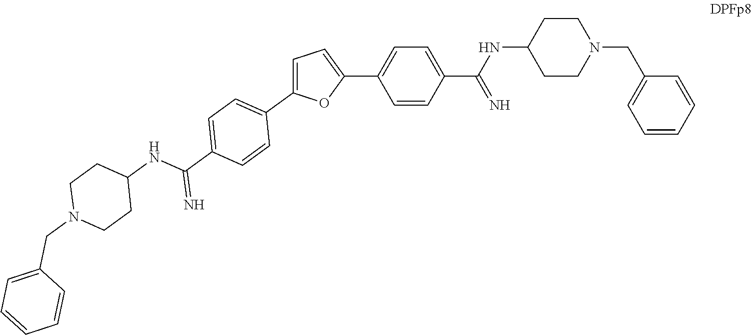 [SMILES: N=C(NC1CCN(Cc2ccccc2)CC1)c1ccc(-c2ccc(-c3ccc(C(=N)NC4CCN(Cc5ccccc5)CC4)cc3)o2)cc1]

or a pharmaceutically acceptable salt thereof, wherein the rSM is covalently bound to L¹ at any available modifiable C, N, or O atom.

In some embodiments, the rSM is a G-quadruplex binder such as the following:

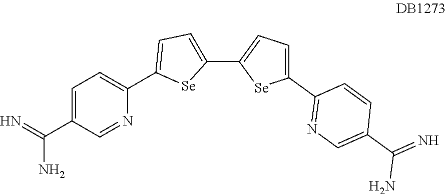 [SMILES: N=C(N)c1ccc(-c2ccc(-c3ccc(-c4ccc(C(=N)N)cn4)[se]3)[se]2)nc1]

or a pharmaceutically acceptable salt thereof, wherein the rSM is covalently bound to L¹ at any available modifiable C or N atom.

In some embodiments, the rSM is one of the following compounds:

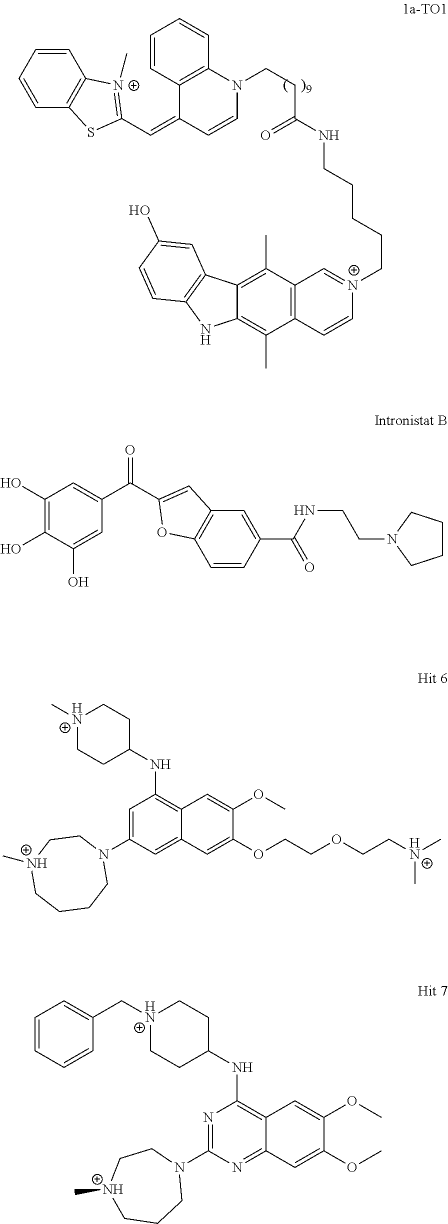

or a pharmaceutically acceptable salt thereof; wherein the rSM is covalently bound to L¹ at any available modifiable C, N, S, or O atom.

In some embodiments, the rSM is selected from one of those described in J. Med Chem. 2018, 61(15), 6501-6517, or U.S. Pat. No. 8,729,263, each of which is hereby incorporated by reference. For example, the rSM is selected from a compound according to Formula I from U.S. Pat. No. 8,729,263:

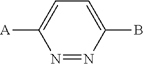

or a pharmaceutically acceptable salt thereof, wherein each variable is as defined therein; and
wherein the rSM is covalently bound to L¹ at any available modifiable C, N, O, S, or P atom.

In some embodiments, the rSM is selected from one of those described in U.S. Pat. No. 9,040,712, which is hereby incorporated by reference. For example, in some embodiments, the rSM is selected from a compound according to Formula X from U.S. Pat. No. 9,040,712:

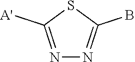

or a pharmaceutically acceptable salt thereof, wherein each variable is as defined therein; and
wherein the rSM is covalently bound to L¹ at any available modifiable C, N, O, S, or P atom.

In some embodiments, the rSM is selected from one of those described in Angelbello, A. J., et al., “Small molecule targeting of RNA structures in neurological disorders,” Annals of the New York Academy of Sciences, 2020 July; 1471(1):57-71, hereby incorporated by reference, or a pharmaceutically acceptable salt thereof; wherein the rSM is covalently bound to L¹ at any available modifiable C, N, O, S, or P atom. In some embodiments, the rSM is one of the following:

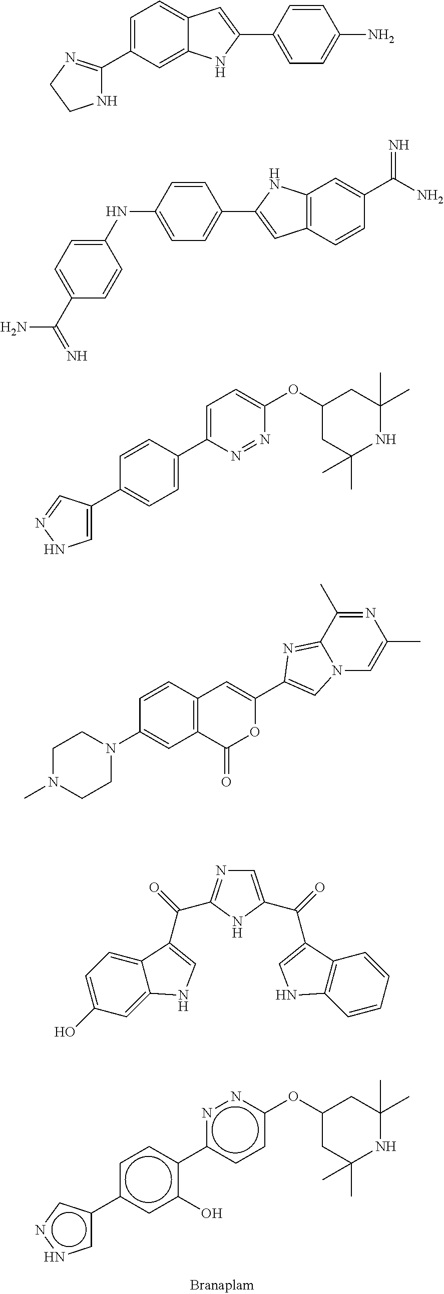

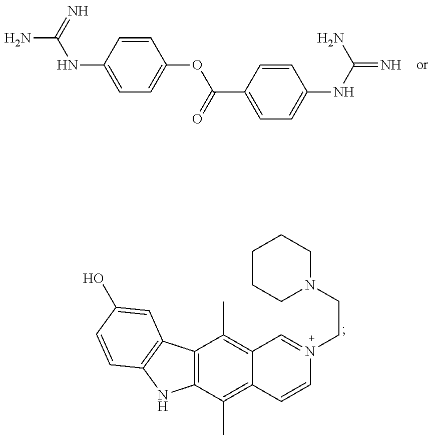

or a pharmaceutically acceptable salt thereof; wherein the rSM is covalently bound to U at any available modifiable C, N, O, S, or P atom.

In some embodiments, the rSM is selected from one of those depicted in Table 2A, below; or a pharmaceutically acceptable salt thereof; wherein the rSM is covalently bound to L¹ at any available modifiable C, N, O, S, or P atom.

In some embodiments, the rSM is a compound according to Formula IX:

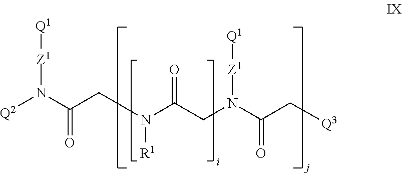

or a pharmaceutically acceptable salt thereof; wherein the rSM is covalently bound to L¹ at any available modifiable C, N, O, S, or P atom; and
wherein each variable is as defined in U.S. Pat. No. 9,150,612, the entirety of which is hereby incorporated by reference.

In some embodiments, the rSM is a compound according to Formula X:

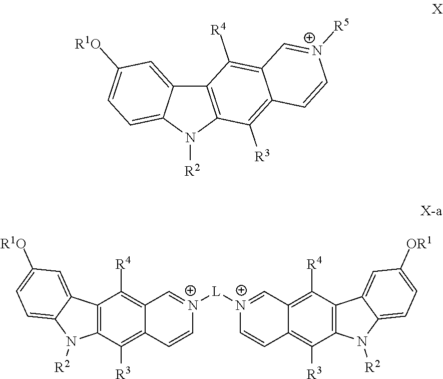

or a pharmaceutically acceptable salt thereof; wherein the rSM is covalently bound to L at any available modifiable C, N, O, S, or P atom; and
wherein each variable is as defined in U.S. Pat. No. 9,550,769, the entirety of which is hereby incorporated by reference. In some embodiments, variable L¹ above is

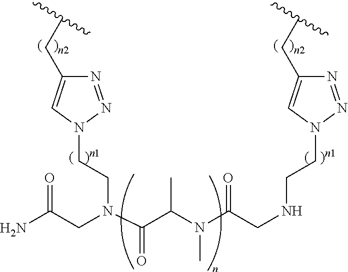

wherein each variable is as defined in U.S. Pat. No. 9,550,769.

In some embodiments, the rSM is selected from one of those disclosed in U.S. Pat. No. 10,157,261, the entirety of which is hereby incorporated by reference; and wherein the rSM is covalently bound to L¹ at any available modifiable C, N, O, S, or P atom.

In some embodiments, the rSM is a compound according to Formula XI:

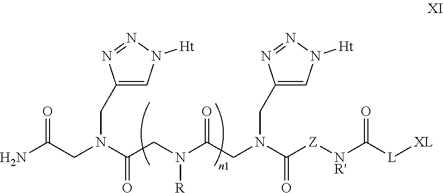

or a pharmaceutically acceptable salt thereof; wherein the rSM is covalently bound to L¹ at any available modifiable C, N, O, S, or P atom; and
wherein each variable is as defined in U.S. Pat. No. 9,586,944, the entirety of which is hereby incorporated by reference.

In some embodiments, the rSM is a compound according to Formula XII:

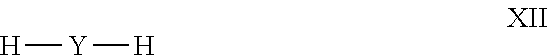

wherein H is a group of the structure

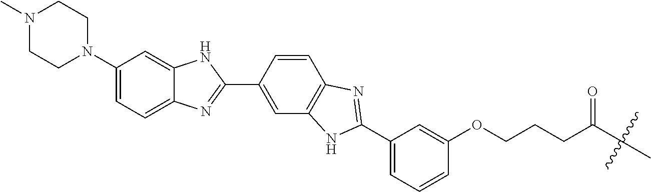 [SMILES: *C(=O)CCCOc1cccc(-c2nc3ccc(-c4nc5ccc(N6CCN(C)CC6)cc5[nH]4)cc3[nH]2)c1]

or a pharmaceutically acceptable salt thereof; wherein the rSM is covalently bound to L¹ at any available modifiable C, N, O, S, or P atom; and
wherein each variable is as defined in U.S. Pat. No. 9,795,687, the entirety of which is hereby incorporated by reference.

In some embodiments, the rSM is a compound selected from one of the following:

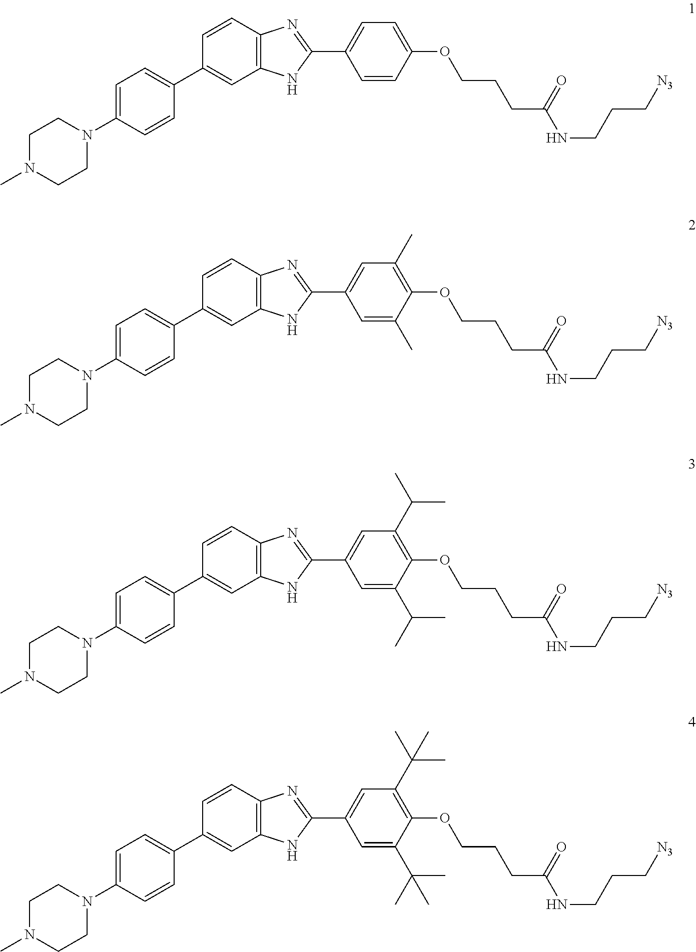 [SMILES: CN1CCN(c2ccc(-c3ccc4nc(-c5ccc(OCCCC(=O)NCCCN=[N+]=[N-])cc5)[nH]c4c3)cc2)CC1 ; Cc1cc(-c2nc3ccc(-c4ccc(N5CCN(C)CC5)cc4)cc3[nH]2)cc(C)c1OCCCC(=O)NCCCN=[N+]=[N-] ; CC(C)c1cc(-c2nc3ccc(-c4ccc(N5CCN(C)CC5)cc4)cc3[nH]2)cc(C(C)C)c1OCCCC(=O)NCCCN=[N+]=[N-] ; CN1CCN(c2ccc(-c3ccc4nc(-c5cc(C(C)(C)C)c(OCCCC(=O)NCCCN=[N+]=[N-])c(C(C)(C)C)c5)[nH]c4c3)cc2)CC1]

or a pharmaceutically acceptable salt thereof; wherein the rSM is covalently bound to L¹ at any available modifiable C, N, O, S, or P atom; or
another compound disclosed in WO 2018/151810, the entirety of which is hereby incorporated by reference.

In some embodiments, the rSM is a compound of the following structure:

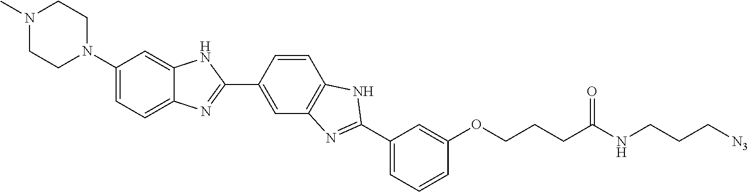 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

or a pharmaceutically acceptable salt thereof; wherein the rSM is covalently bound to U at any available modifiable C, N, O, S, or P atom;
or another compound disclosed in in WO 2018/152414, the entirety of which is hereby incorporated by reference.

In some embodiments, the rSM is a compound of the following structure:

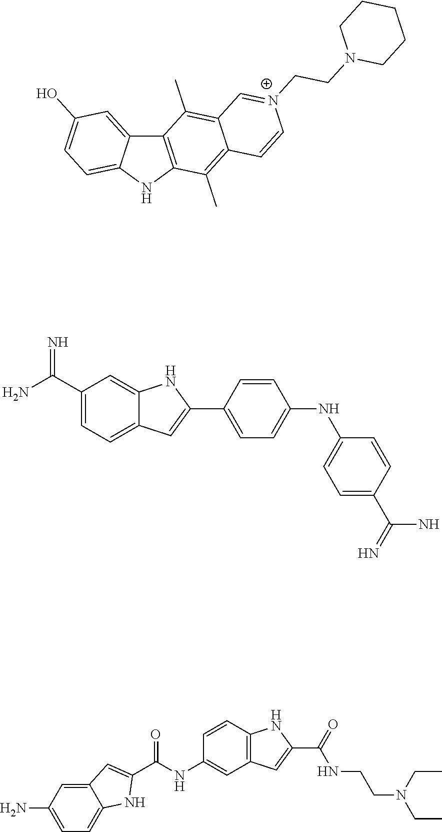 [SMILES: Cc1c2cc[n+](CCN3CCCCC3)cc2c(C)c2c1[nH]c1ccc(O)cc12 ; N=C(N)c1ccc(Nc2ccc(-c3cc4ccc(C(=N)N)cc4[nH]3)cc2)cc1 ; CCN(CC)CCNC(=O)c1cc2cc(NC(=O)c3cc4cc(N)ccc4[nH]3)ccc2[nH]1]

or a pharmaceutically acceptable salt thereof; wherein the rSM is covalently bound to U at any available modifiable C, N, O, S, or P atom; or
another compound disclosed in US 2018/0334678, the entirety of which is hereby incorporated by reference.

In some embodiments, the rSM is a compound according to Formula XIII:

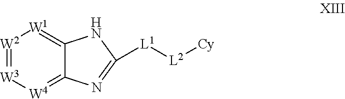

or a pharmaceutically acceptable salt thereof; wherein the rSM is covalently bound to U at any available modifiable C, N, O, S, or P atom; and
wherein each variable is as defined in US 2018/0296532, the entirety of which is hereby incorporated by reference.

In some embodiments, the rSM is a compound according to Formula XIV:

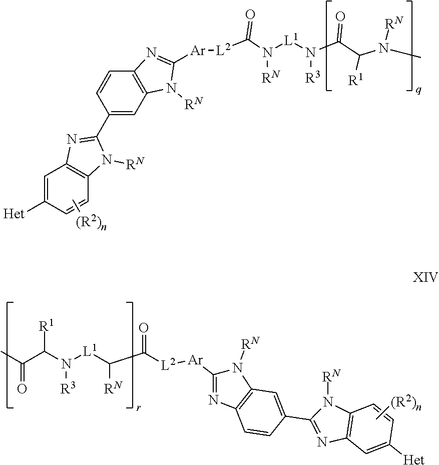

or a pharmaceutically acceptable salt thereof; wherein the rSM is covalently bound to L¹ at any available modifiable C, N, O, S, or P atom; and
wherein each variable is as defined in WO 2018/098297, the entirety of which is hereby incorporated by reference.

In some embodiments, the rSM is a compound according to Formula XV, XVI, or XVII:

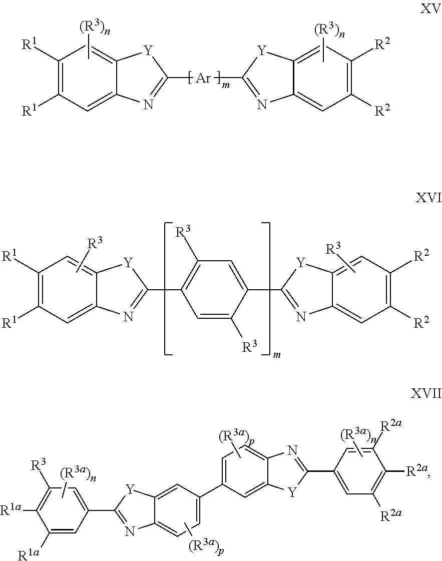

or a pharmaceutically acceptable salt thereof, wherein the rSM is covalently bound to L¹ at any available modifiable C, N, O, S, or P atom; and
wherein each variable is as defined in US 2019/0152924, the entirety of which is hereby incorporated by reference.

In some embodiments, the rSM is a compound according to Formula XVIII:

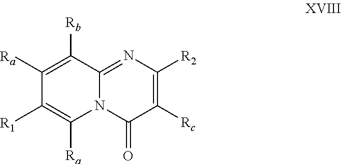

or a pharmaceutically acceptable salt thereof; wherein the rSM is covalently bound to L¹ at any available modifiable C, N, O, S, or P atom; and
wherein each variable is as defined in WO 2019/005993, the entirety of which is hereby incorporated by reference.

In some embodiments, the rSM is a compound according to Formula XIX:

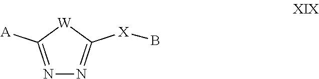

or a pharmaceutically acceptable salt thereof, wherein the rSM is covalently bound to L at any available modifiable C, N, O, S, or P atom; and
wherein each variable is as defined in WO 2018/232039, the entirety of which is hereby incorporated by reference.

In some embodiments, the rSM is a compound according to Formula XX:

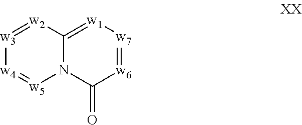

or a pharmaceutically acceptable salt thereof, wherein the rSM is covalently bound to L¹ at any available modifiable C, N, O, S, or P atom; and
wherein each variable is as defined in WO 2019/005980, the entirety of which is hereby incorporated by reference.

In some embodiments, the rSM is a compound according to Formula XXI:

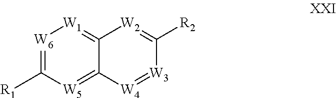

or a pharmaceutically acceptable salt thereof, wherein the rSM is covalently bound to L¹ at any available modifiable C, N, O, S, or P atom; and
wherein each variable is as defined in WO 2018/226622, the entirety of which is hereby incorporated by reference.

In some embodiments, the rSM is a compound according to Formula XXII:

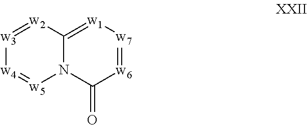

or a pharmaceutically acceptable salt thereof; wherein the rSM is covalently bound to L¹ at any available modifiable C, N, O, S, or P atom; and
wherein each variable is as defined in WO 2018/098446, the entirety of which is hereby incorporated by reference.

In some embodiments, the rSM is a compound according to Formula XXIII:

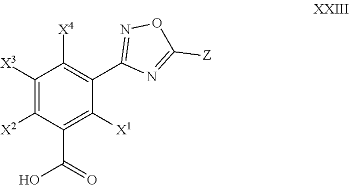

or a pharmaceutically acceptable salt thereof; wherein the rSM is covalently bound to L¹ at any available modifiable C, N, O, S, or P atom; and
wherein each variable is as defined in WO 2017/087364, the entirety of which is hereby incorporated by reference.

In some embodiments, the rSM is ataluren:

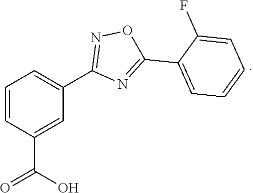 [SMILES: O=C(O)c1cccc(-c2noc(-c3ccccc3F)n2)c1]

or a deuterated analog thereof or pharmaceutically acceptable salt thereof, disclosed in US 2018/0333397 or WO 2017/087364, each of which is hereby incorporated by reference.

In some embodiments, the rSM is a compound of the following structure:

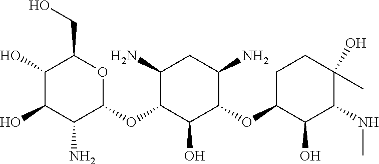 [SMILES: CN[C@@H]1[C@@H](O)[C@@H](O[C@@H]2[C@@H](O)[C@H](O[C@H]3O[C@H](CO)[C@@H](O)[C@H](O)[C@H]3N)[C@@H](N)C[C@H]2N)CC[C@]1(C)O]

or a pharmaceutically acceptable salt thereof, wherein the rSM is covalently bound to L¹ at any available modifiable C, N, O, S, or P atom; as described in US 2018/147228, the entirety of which is hereby incorporated by reference.

In some embodiments, the rSM is a compound according to Formula XXIV:

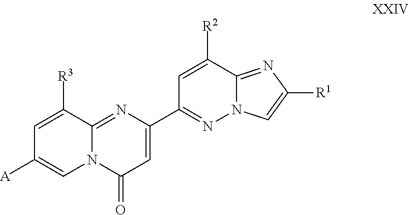

or a pharmaceutically acceptable salt thereof; wherein the rSM is covalently bound to L¹ at any available modifiable C, N, O, S, or P atom; and
wherein each variable is as defined in U.S. Pat. No. 9,969,754, the entirety of which is hereby incorporated by reference.

In some embodiments, the rSM is a compound according to Formula XXV-ii:

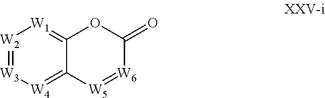

or a pharmaceutically acceptable salt thereof; wherein the rSM is covalently bound to L¹ at any available modifiable C, N, O, S, or P atom; and
wherein each variable is as defined in U.S. Pat. No. 9,371,336, the entirety of which is hereby incorporated by reference. In some embodiments, the rSM is a compound disclosed in U.S. 9,371,336, or a pharmaceutically acceptable salt thereof.

In some embodiments, the rSM is a compound according to Formula XXV-ii:

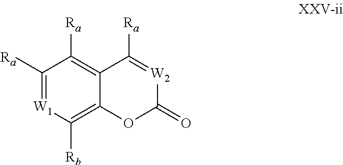

or a pharmaceutically acceptable salt thereof; wherein the rSM is covalently bound to L¹ at any available modifiable C, N, O, S, or P atom; and
wherein each variable is as defined in U.S. Pat. No. 9,617,268, the entirety of which is hereby incorporated by reference. In some embodiments, the rSM is a compound disclosed in U.S. Pat. No. 9,617,268, or a pharmaceutically acceptable salt thereof.

In some embodiments, the rSM is a compound according to Formula XXVI:

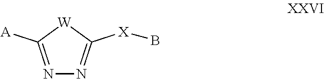

or a pharmaceutically acceptable salt thereof; wherein the rSM is covalently bound to L¹ at any available modifiable C, N, O, S, or P atom; and
wherein each variable is as defined in US 2019/0000844, the entirety of which is hereby incorporated by reference. In some embodiments, the rSM is a compound disclosed in US 2019/0000844, or a pharmaceutically acceptable salt thereof.

In some embodiments, the rSM is a compound according to Formula XXVII:

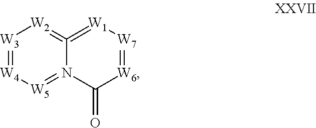

or a pharmaceutically acceptable salt thereof; wherein the rSM is covalently bound to L¹ at any available modifiable C, N, O, S, or P atom; and
wherein each variable is as defined in US 2018/0161456, the entirety of which is hereby incorporated by reference. In some embodiments, the rSM is a compound disclosed in US 2018/0161456, or a pharmaceutically acceptable salt thereof.

In some embodiments, the rSM is a compound according to Formula XXVIII:

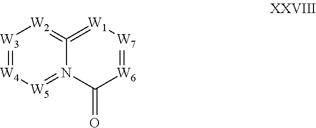

or a pharmaceutically acceptable salt thereof; wherein the rSM is covalently bound to L¹ at any available modifiable C, N, O, S, or P atom; and
wherein each variable is as defined in U.S. Pat. No. 10,195,202, the entirety of which is hereby incorporated by reference. In some embodiments, the rSM is a compound disclosed in U.S. Pat. No. 10,195,202, or a pharmaceutically acceptable salt thereof.

In some embodiments, the rSM is a compound according to one of Formulae XXIX-XXXIII:

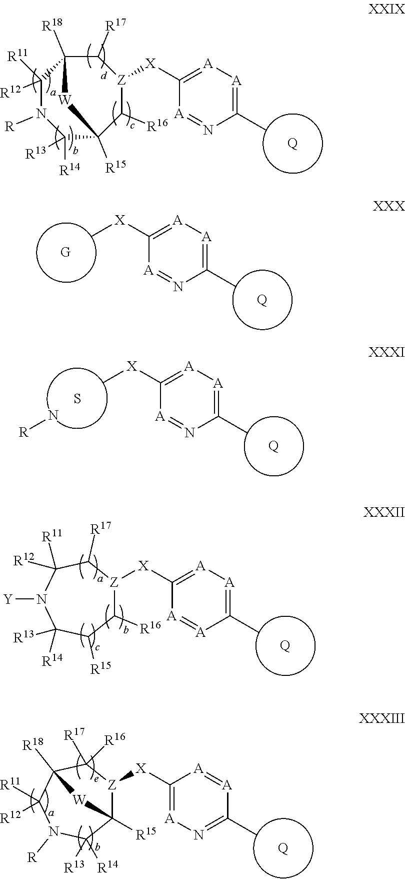

or a pharmaceutically acceptable salt thereof; wherein the rSM is covalently bound to L¹ at any available modifiable C, N, O, S, or P atom; and
wherein each variable is as defined in WO 2019/028440, the entirety of which is hereby incorporated by reference. In some embodiments, the rSM is a compound disclosed in WO 2019/028440, or a pharmaceutically acceptable salt thereof.

In some embodiments, the rSM is a compound according to one of Formulae XXXIV-XLXI:

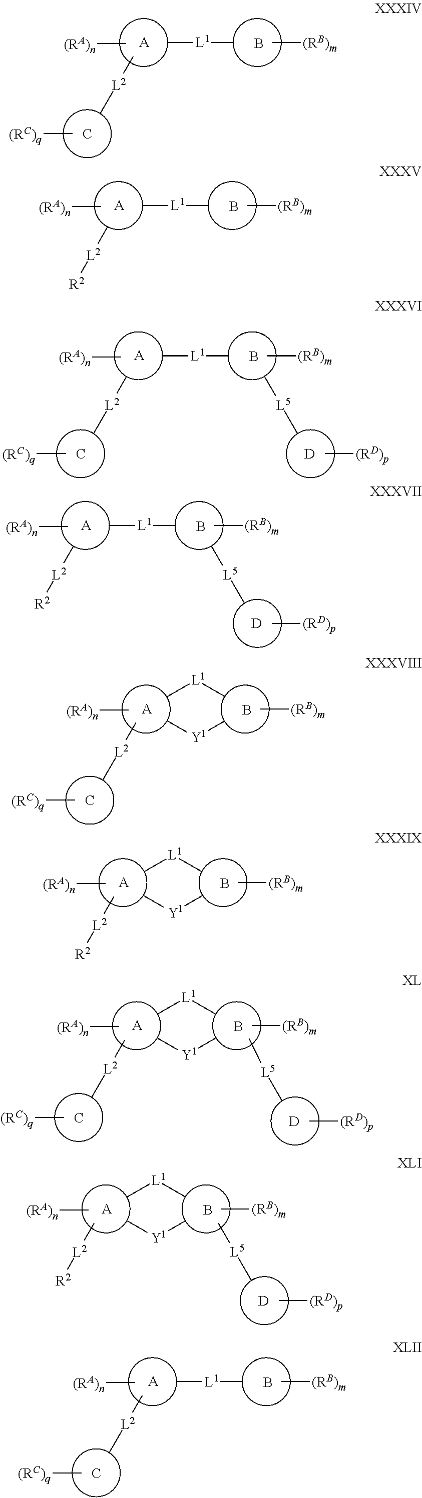

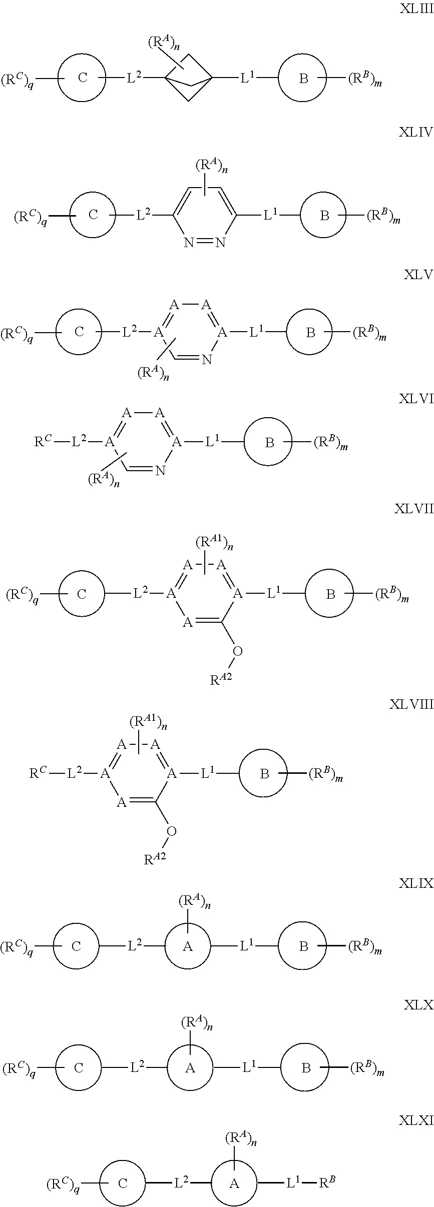

or a pharmaceutically acceptable salt thereof; wherein the rSM is covalently bound to L¹ at any available modifiable C, N, O, S, or P atom; and
wherein each variable is as defined in WO 2019/060917, the entirety of which is hereby incorporated by reference. In some embodiments, the rSM is a compound disclosed in WO 2019/060917, or a pharmaceutically acceptable salt thereof.

In some embodiments, the rSM is a compound according to Formula XLXII or XLXIII:

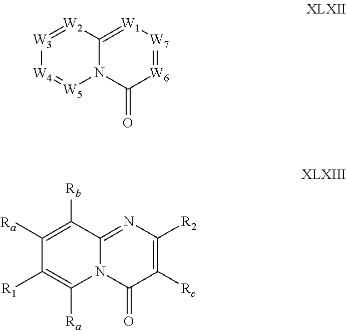

or a pharmaceutically acceptable salt thereof, wherein the rSM is covalently bound to L¹ at any available modifiable C, N, O, S, or P atom; and
wherein each variable is as defined in U.S. Pat. No. 9,879,007, the entirety of which is hereby incorporated by reference.

In some embodiments, the rSM is a compound according to Formula XLXIV or XLXV:

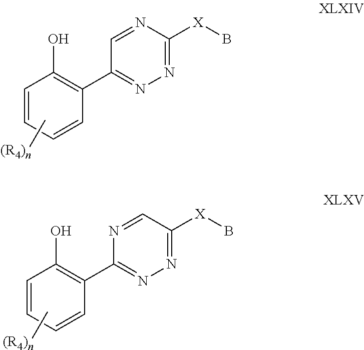

or a pharmaceutically acceptable salt thereof; wherein the rSM is covalently bound to L¹ at any available modifiable C, N, O, S, or P atom; and
wherein each variable is as defined in WO 2019/191229, the entirety of which is hereby incorporated by reference.

In some embodiments, the rSM is a compound according to Formula XLXVI:

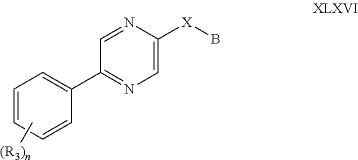

or a pharmaceutically acceptable salt thereof; wherein the rSM is covalently bound to L¹ at any available modifiable C, N, O, S, or P atom; and
wherein each variable is as defined in WO 2019/191092, the entirety of which is hereby incorporated by reference.

In some embodiments, the rSM is a compound according to Formula XLXVII:

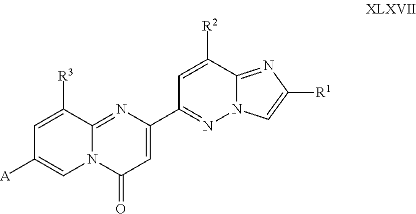

or a pharmaceutically acceptable salt thereof; wherein the rSM is covalently bound to L¹ at any available modifiable C, N, O, S, or P atom; and
wherein each variable is as defined in US 2019/315773, the entirety of which is hereby incorporated by reference.

In some embodiments, the rSM is a compound according to Formula LVIII, LIX, or LX:

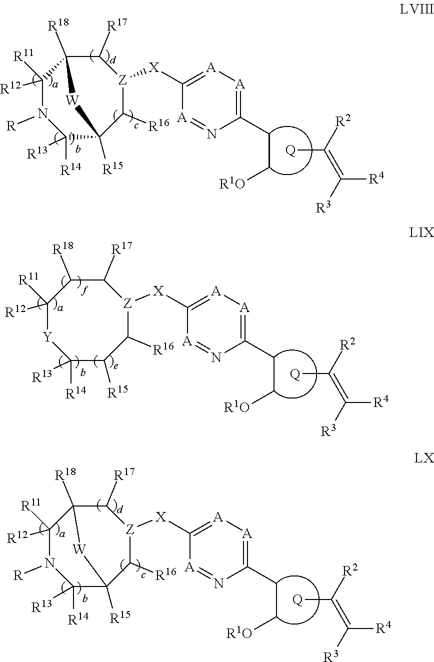

or a pharmaceutically acceptable salt thereof; wherein the rSM is covalently bound to L) at any available modifiable C, N, O, S, or P atom; and
wherein each variable is as defined in WO 2019/199972, the entirety of which is hereby incorporated by reference. Such compounds are useful, for example, in modulating splicing of the FOXM1 gene for use in the treatment of cancer.

In some embodiments, the rSM is a compound according to Formula LXI:

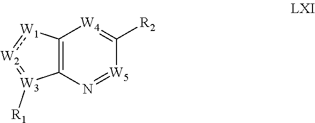

or a pharmaceutically acceptable salt thereof; wherein the rSM is covalently bound to L¹ at any available modifiable C, N, O, S, or P atom; and
wherein each variable is as defined for Formula (I) in WO 2020/005873, the entirety of which is hereby incorporated by reference. Such compounds are useful, for example, in modulating RNA targets that mediate Huntington's disease. In some embodiments, the compound is of formula (Ibb1) described therein:

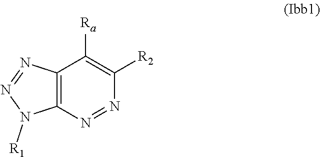

wherein or a pharmaceutically acceptable salt thereof; wherein the rSM is covalently bound to L¹ at any available modifiable C, N, O, S, or P atom; and wherein each variable is as defined therein.

In some embodiments, the rSM is a compound according to Formula LXII or LXIII:

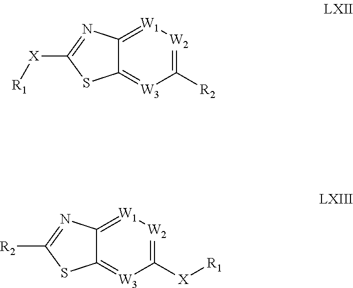

or a pharmaceutically acceptable salt thereof; wherein the rSM is covalently bound to L¹ at any available modifiable C, N, O, S, or P atom; and
wherein each variable is as defined in WO 2020/005877, the entirety of which is hereby incorporated by reference. Such compounds are useful, for example, in binding to HTT RNA transcripts for use in the treatment of diseases such as Huntington's.

In some embodiments, the rSM is a compound according to Formula LXIV, LXV, LXVI, or LXVII:

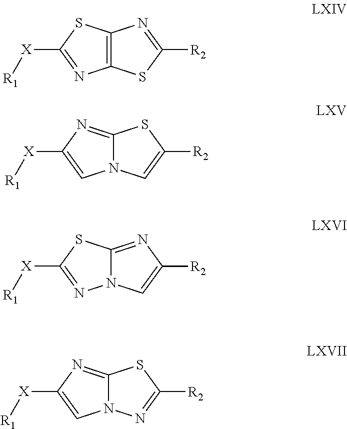

or a pharmaceutically acceptable salt thereof, wherein the rSM is covalently bound to L¹ at any available modifiable C, N, O, S, or P atom; and wherein each variable is as defined in WO 2020/005882, the entirety of which is hereby incorporated by reference. Such compounds are useful, for example, in binding to HTT RNA transcripts for use in the treatment of diseases such as Huntington's.

In some embodiments, the rSM is selected from one of those depicted in U.S. Pat. Nos. 8,729,263, 9,545,404, 9,856,474, or 7,838,657, each of which is hereby incorporated by reference.

In some embodiments, the rSM is selected from one of those depicted in Table 2, below; or a pharmaceutically acceptable salt thereof, wherein the rSM is covalently bound to L¹ at any available modifiable C, N, O, S, or P atom.

TABLE 2
Additional rSMs

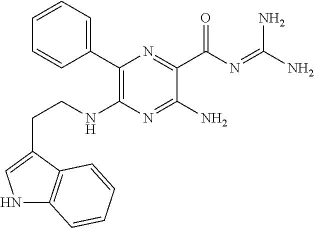 [SMILES: NC(N)=NC(=O)c1nc(-c2ccccc2)c(NCCc2c[nH]c3ccccc23)nc1N]

Patwardhan et al., Med. Chem. Comm. 2017,
8, 1022-1036; hereby incorporated by
reference.

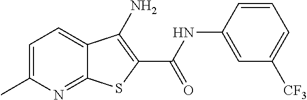 [SMILES: Cc1ccc2c(N)c(C(=O)Nc3cccc(C(F)(F)F)c3)sc2n1]

Sztuba-Solinska et al., J. Am. Chem. Soc.
2014, 136, 8402-8410; hereby incorporated
by reference.

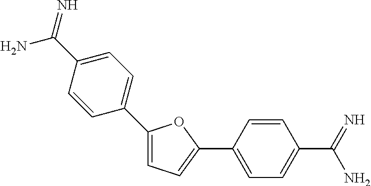 [SMILES: N=C(N)c1ccc(-c2ccc(-c3ccc(C(=N)N)cc3)o2)cc1]

Donlic et al., Ange. Chem. Int. Ed. 2018, 40,
13242-13247; hereby incorporated by
reference.

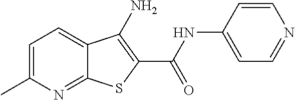 [SMILES: Cc1ccc2c(N)c(C(=O)Nc3ccncc3)sc2n1]

Sztuba-Solinska et al., J. Am. Chem. Soc.
2014, 136, 8402-8410.

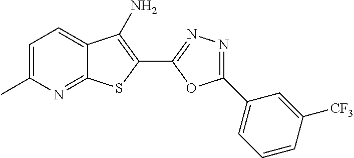 [SMILES: Cc1ccc2c(N)c(-c3nnc(-c4cccc(C(F)(F)F)c4)o3)sc2n1]

Sztuba-Solinska et al., J. Am. Chem. Soc.
2014, 136, 8402-8410.

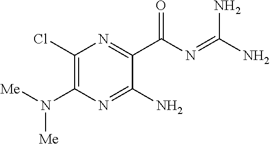 [SMILES: CN(C)c1nc(N)c(C(=O)N=C(N)N)nc1Cl]

Patwardhan et al. 2017

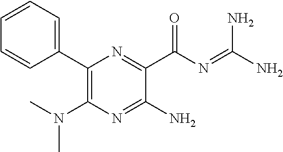 [SMILES: CN(C)c1nc(N)c(C(=O)N=C(N)N)nc1-c1ccccc1]

Patwardhan et al. 2017

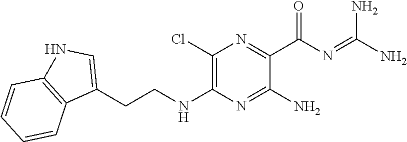 [SMILES: NC(N)=NC(=O)c1nc(Cl)c(NCCc2c[nH]c3ccccc23)nc1N]

Patwardhan et al. 2017

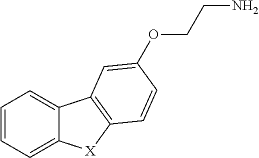

X = N, O, S
Connelly, et al., Nature communications,
2019; 10(1):1501; hereby incorporated by
reference

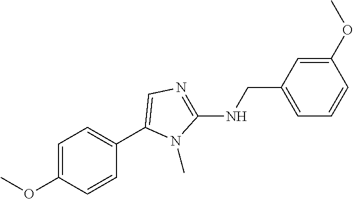 [SMILES: COc1ccc(-c2cnc(NCc3cccc(OC)c3)n2C)cc1]

Abulwerdi; et al., ACS Chem. Biol. 2019, 14,
223-235; hereby incorporated by reference.

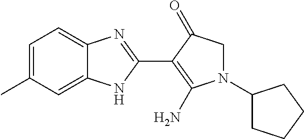 [SMILES: Cc1ccc2nc(C3=C(N)N(C4CCCC4)CC3=O)[nH]c2c1]

Abulwerdi; et al., ACS Chem. Biol. 2019, 14,
223-235; hereby incorporated by reference.

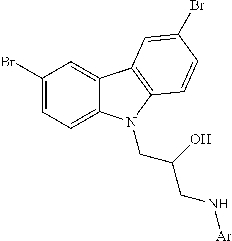

Ar = carbocyclic, heterocyclic
Connelly et al., ACS Chem Biol. 2017, 12(2):
435-443; hereby incorporated by reference.

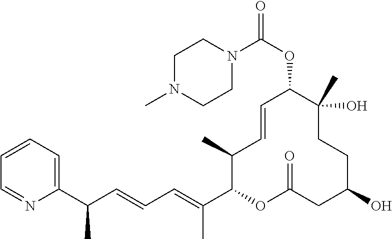 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C]

Seiler et al., Nature Medicine 2018, 24, 497-
504; hereby incorporated by reference.

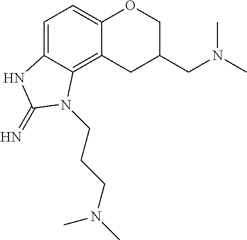 [SMILES: CN(C)CCCn1c(=N)[nH]c2ccc3c(c21)CC(CN(C)C)CO3]

Charrette et al., ACS Chem. Biol. 2016,
11(1), 3263-3267; hereby incorporated by
reference.

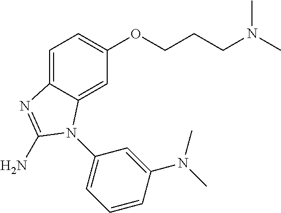 [SMILES: CN(C)CCCOc1ccc2nc(N)n(-c3cccc(N(C)C)c3)c2c1]

Charette et al. 2016

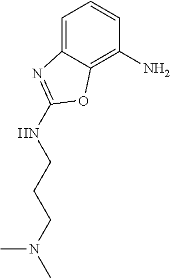 [SMILES: CN(C)CCCNc1nc2cccc(N)c2o1]

Charette et al. 2016

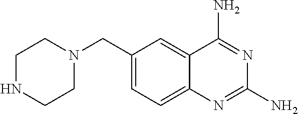 [SMILES: Nc1nc(N)c2cc(CN3CCNCC3)ccc2n1]

Charette et al. 2016

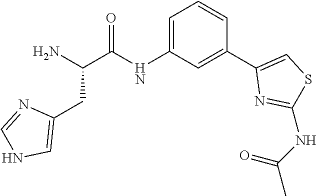 [SMILES: CC(=O)Nc1nc(-c2cccc(NC(=O)[C@@H](N)Cc3c[nH]cn3)c2)cs1]

Joly, J..P. et al., Chemistry 2014, 20, 2071-
2079; hereby incorporated by reference.

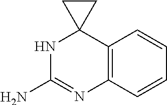 [SMILES: NC1=Nc2ccccc2C2(CC2)N1]

Charette et al. 2016

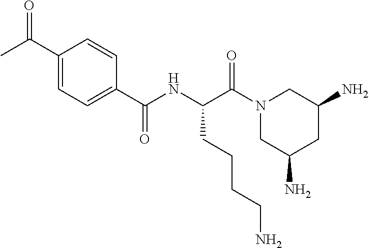 [SMILES: CC(=O)c1ccc(C(=O)N[C@@H](CCCCN)C(=O)N2C[C@H](N)C[C@H](N)C2)cc1]

Charette et al. 2016

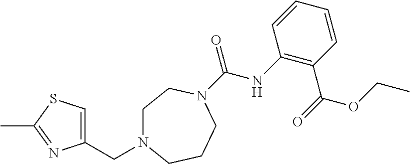 [SMILES: CCOC(=O)c1ccccc1NC(=O)N1CCCN(Cc2csc(C)n2)CC1]

Park et al., J. Am. Chem. Soc. 2011, 133,
10094-10100; hereby incorporated by
reference.

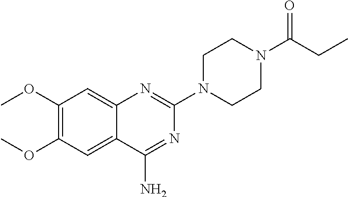 [SMILES: CCC(=O)N1CCN(c2nc(N)c3cc(OC)c(OC)cc3n2)CC1]

Bottini, A. et al., Chem. Biol. Drug Des.
2015, 86, 663-673; hereby incorporated by
reference.

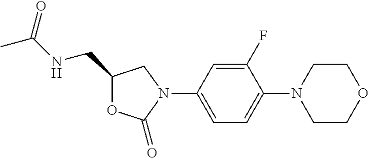 [SMILES: CC(=O)NC[C@H]1CN(c2ccc(N3CCOCC3)c(F)c2)C(=O)O1]

Lin et al., Antimicrob. Agents
Chemother. 1997, 41, 2127-2131; hereby
incorporated by reference.

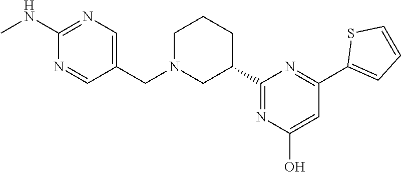 [SMILES: CNc1ncc(CN2CCC[C@H](c3nc(O)cc(-c4cccs4)n3)C2)cn1]

Howe, J. A. et al., Nature 2015, 526, 672-
677; hereby incorporated by reference.

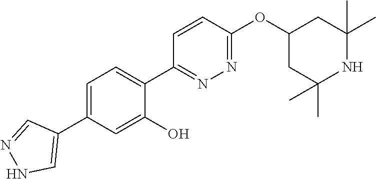 [SMILES: CC1(C)CC(Oc2ccc(-c3ccc(-c4cn[nH]c4)cc3O)nn2)CC(C)(C)N1]

Palacino, J. Et al., Nat. Chem. Biol. 2015, 11,
511-517; hereby incorporated by reference.

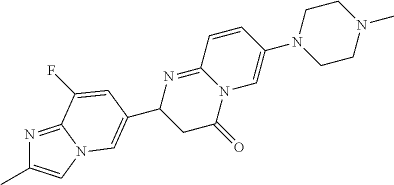

Sivaramakrishnan, M. et al., Nat. Commun.
2017, 8, 1476; hereby incorporated by
reference3.

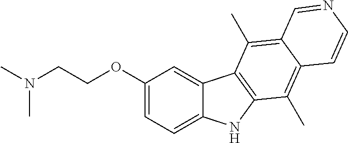 [SMILES: Cc1c2ccncc2c(C)c2c1[nH]c1ccc(OCCN(C)C)cc12]

R. V. Brown, et al., J. Am. Chem. Soc. 2017,
139, 7456-7475; hereby incorporated by
reference.

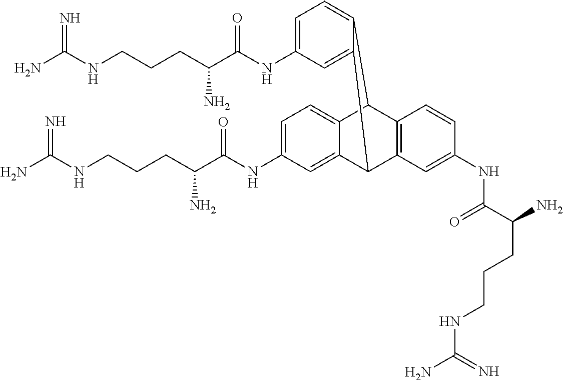 [SMILES: N=C(N)NCCC[C@@H](N)C(=O)Nc1ccc2c(c1)C1c3cc(NC(=O)[C@H](N)CCCNC(=N)N)ccc3C2c2ccc(NC(=O)[C@@H](N)CCCNC(=N)N)cc21]

Barros, S. A., et al., Angew. Chem. Int. Ed.
Eng. 2016, 55, 8258-8261; hereby
incorporated by reference.

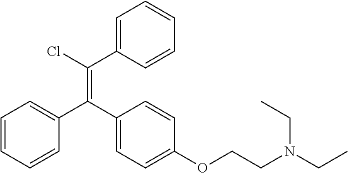 [SMILES: CCN(CC)CCOc1ccc(/C(=C(/Cl)c2ccccc2)c2ccccc2)cc1]

Prado, S. et al., Biochem. Pharmacol. 2016,
107, 14-28; hereby incorporated by reference.

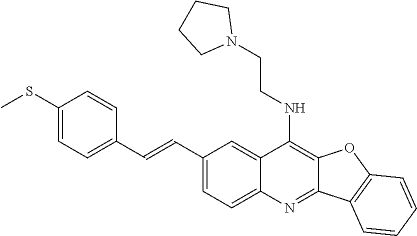 [SMILES: CSc1ccc(/C=C/c2ccc3nc4c(oc5ccccc54)c(NCCN4CCCC4)c3c2)cc1]

Peng et al., J. Med. Chem. 2018, 61, 6629-
6646; hereby incorporated by reference.

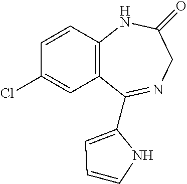 [SMILES: O=C1CN=C(c2ccc[nH]2)c2cc(Cl)ccc2N1]

Mei, H. Y., et al., Bioorg Med Chem. 1997,
5:1173-1184; hereby incorporated by
reference.

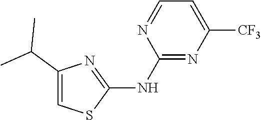 [SMILES: CC(C)c1csc(Nc2nccc(C(F)(F)F)n2)n1]

Garavis, M., et al., ACS Chem. Biol. 2014, 9,
1559-1566; hereby incorporated by
reference.

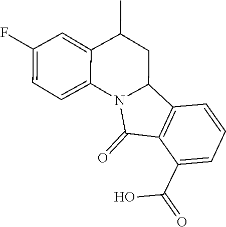 [SMILES: CC1CC2c3cccc(C(=O)O)c3C(=O)N2c2ccc(F)cc21]

Garavis, M., et al., 2014

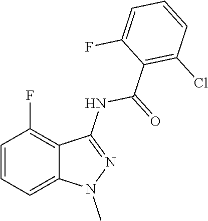 [SMILES: Cn1nc(NC(=O)c2c(F)cccc2Cl)c2c(F)cccc21]

Garavis, M., et al., 2014

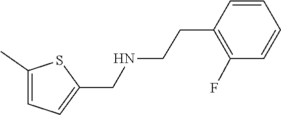 [SMILES: Cc1ccc(CNCCc2ccccc2F)s1]

Garavis, M., et al., 2014

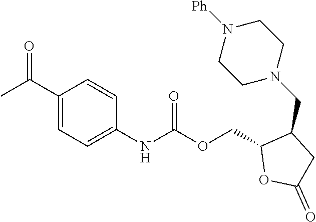 [SMILES: CC(=O)c1ccc(NC(=O)OC[C@H]2OC(=O)C[C@@H]2CN2CCN(c3ccccc3)CC2)cc1]

Orac, C. M., et al., J. Med. Chem. 2011, 54,
6786-6795; hereby incorporated by
reference.

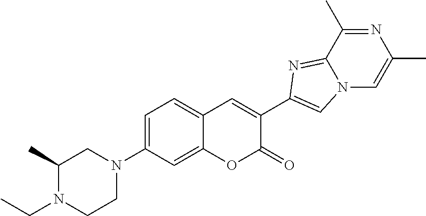 [SMILES: CCN1CCN(c2ccc3cc(-c4cn5cc(C)nc(C)c5n4)c(=O)oc3c2)C[C@@H]1C]

Wang et al., PNAS 2018, 115 (20), E4604-
E4612; hereby incorporated by reference.

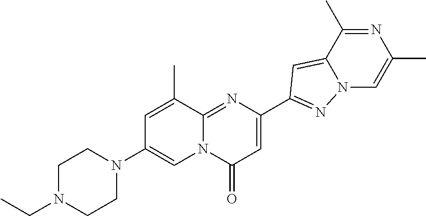 [SMILES: CCN1CCN(c2cc(C)c3nc(-c4cc5c(C)nc(C)cn5n4)cc(=O)n3c2)CC1]

Wang et al., 2018

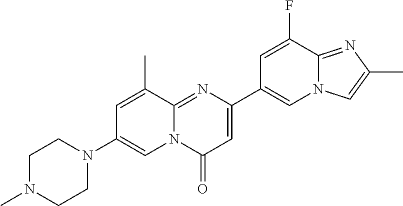

Wang et al., 2018

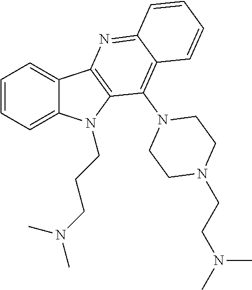 [SMILES: CN(C)CCCn1c2ccccc2c2nc3ccccc3c(N3CCN(CCN(C)C)CC3)c21]

L.D. Smith et al., Cell Rep. 2014, 9, 193-
205; hereby incorporated by reference.

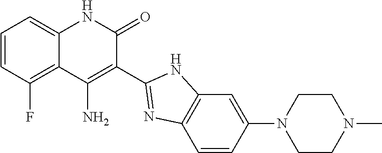 [SMILES: CN1CCN(c2ccc3nc(-c4c(N)c5c(F)cccc5[nH]c4=O)[nH]c3c2)CC1]

Velagapudi, et al., Cell Chem. Bio. 2018, 25,
1086-1094; hereby incorporated by reference.

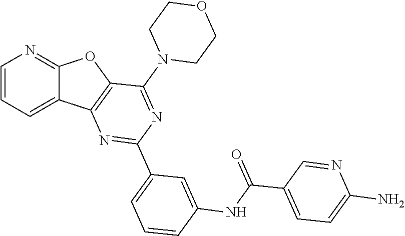 [SMILES: Nc1ccc(C(=O)Nc2cccc(-c3nc(N4CCOCC4)c4oc5ncccc5c4n3)c2)cn1]

Velagapudi, et al., 2018

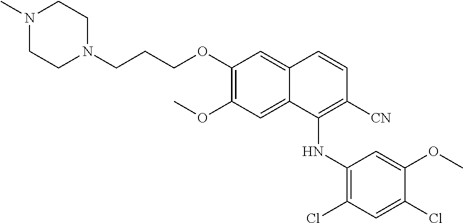

Velagapudi, et al., 2018

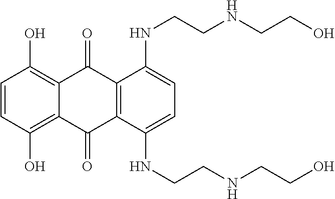 [SMILES: O=C1c2c(O)ccc(O)c2C(=O)c2c(NCCNCCO)ccc(NCCNCCO)c21]

Velagapudi, et al., 2018

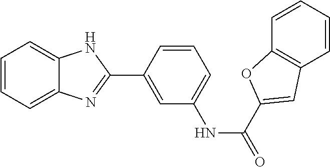 [SMILES: O=C(Nc1cccc(-c2nc3ccccc3[nH]2)c1)c1cc2ccccc2o1]

Velagapudi, et al., 2018

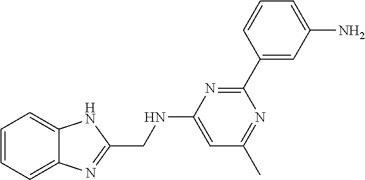 [SMILES: Cc1cc(NCc2nc3ccccc3[nH]2)nc(-c2cccc(N)c2)n1]

Velagapudi, et al., 2018

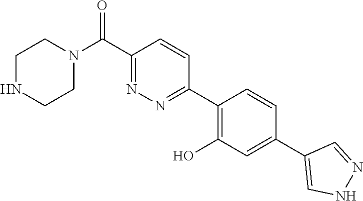 [SMILES: O=C(c1ccc(-c2ccc(-c3cn[nH]c3)cc2O)nn1)N1CCNCC1]

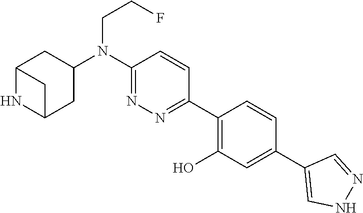 [SMILES: Oc1cc(-c2cn[nH]c2)ccc1-c1ccc(N(CCF)C2CC3CC(C2)N3)nn1]

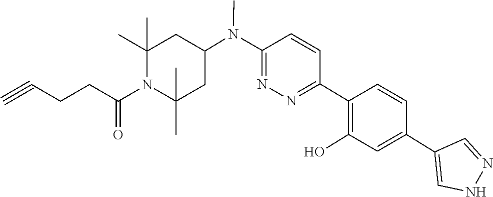

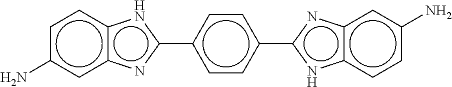

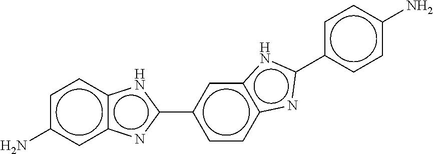

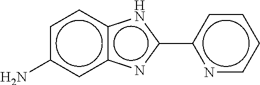

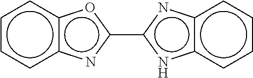

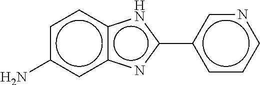

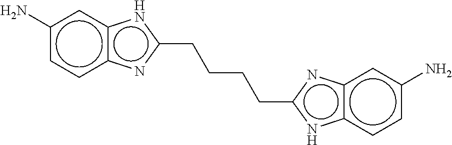

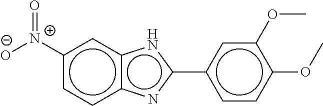

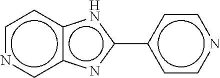

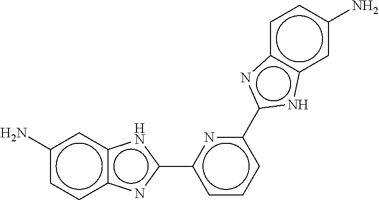

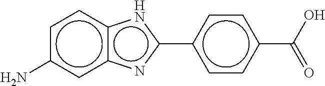

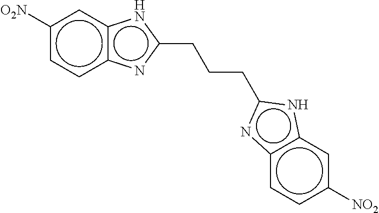

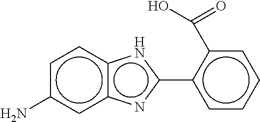

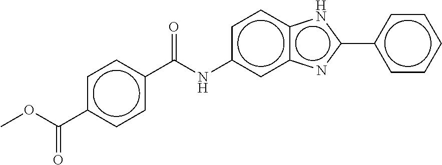

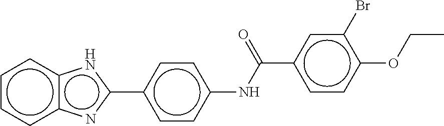

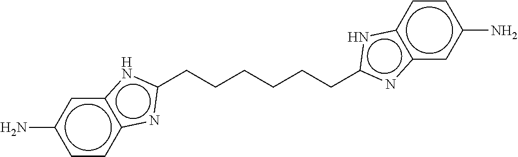

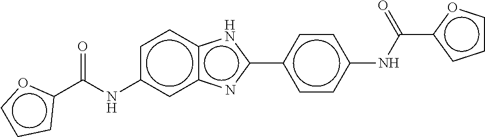

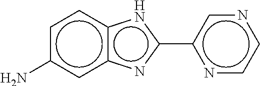

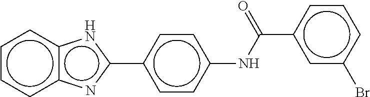

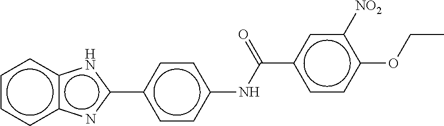

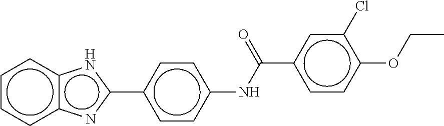

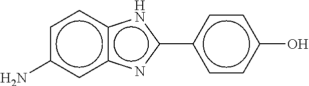

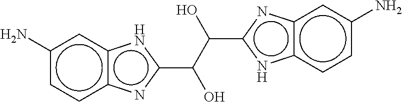

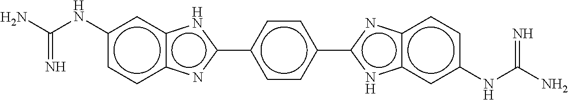

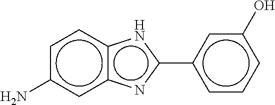

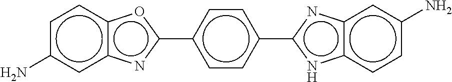

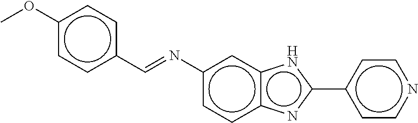

=0

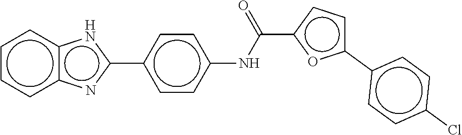

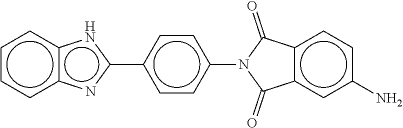

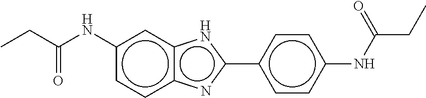

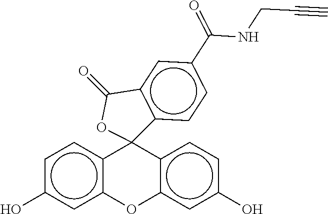

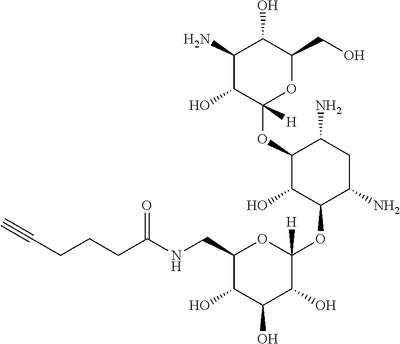 [SMILES: C#CCCCC(=O)NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](O)[C@@H](O)[C@@H]1O]

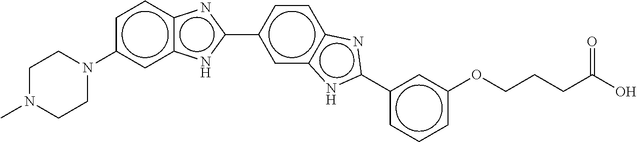

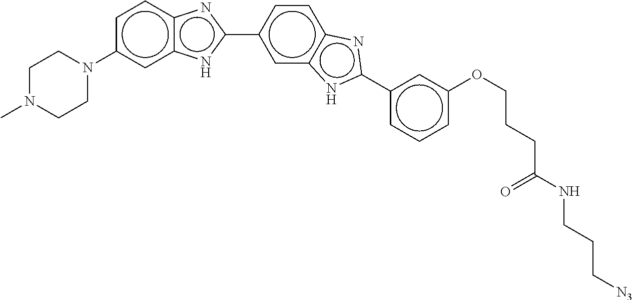

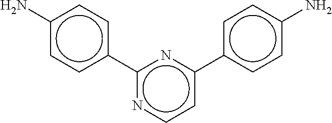

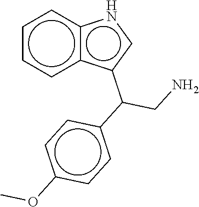

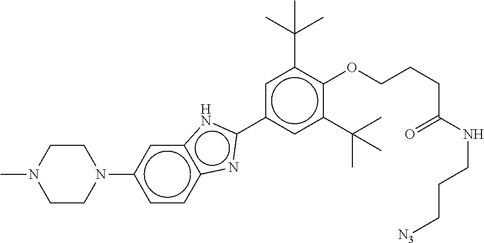

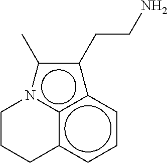

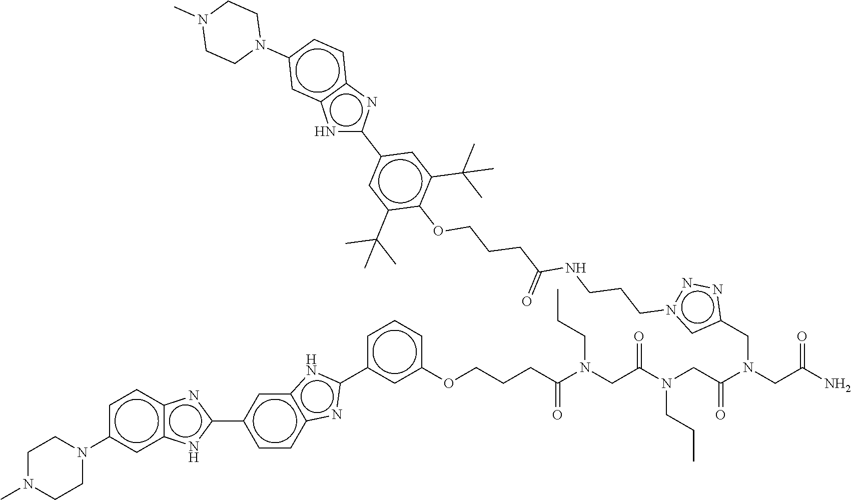

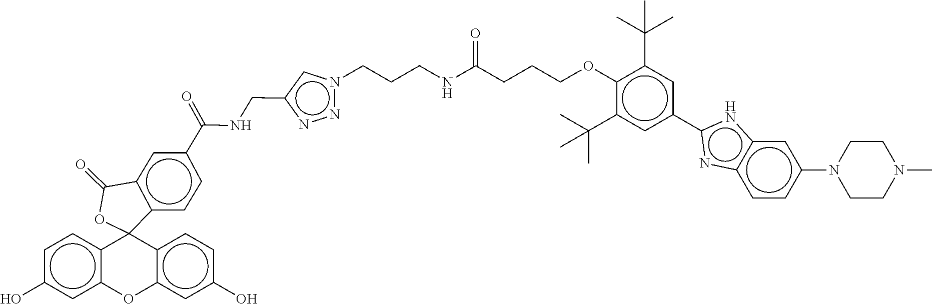

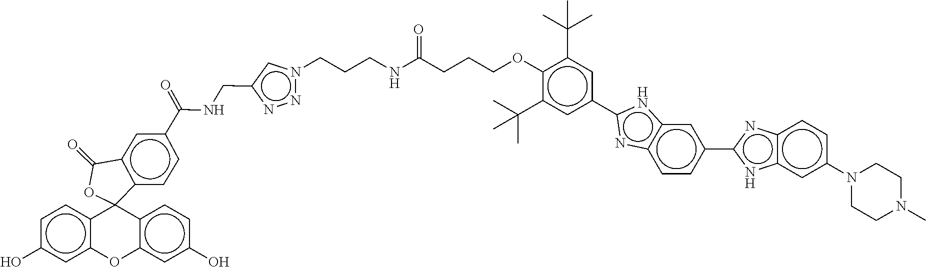

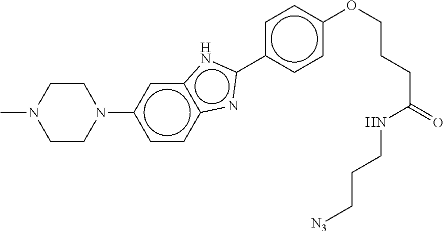

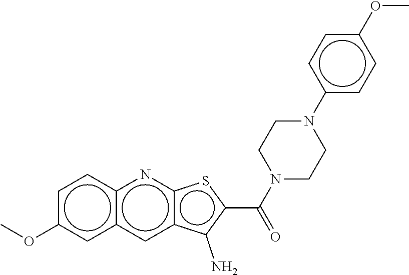

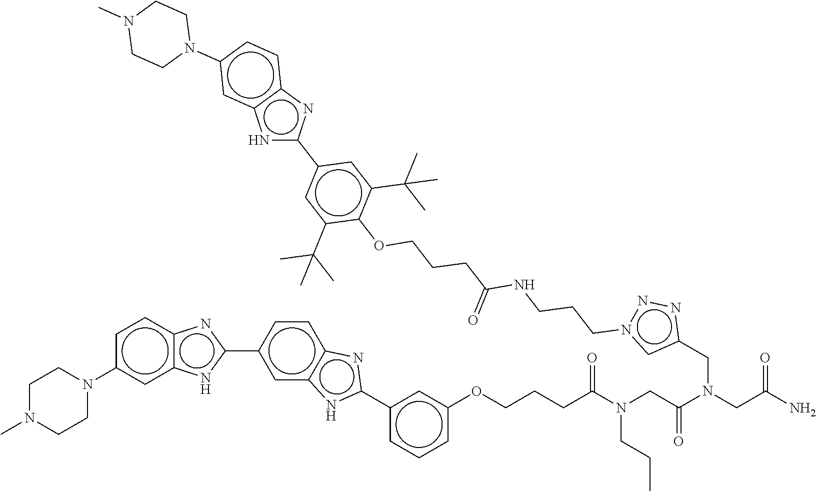

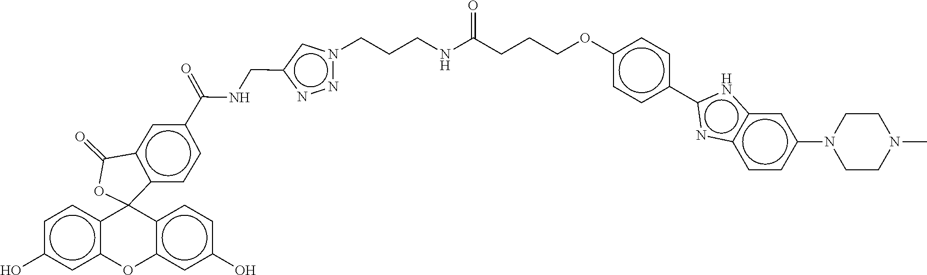

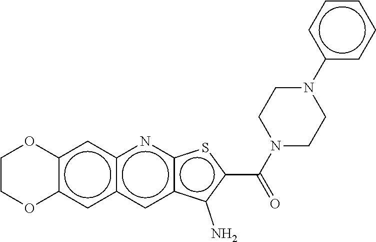

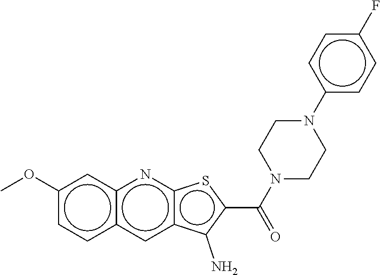

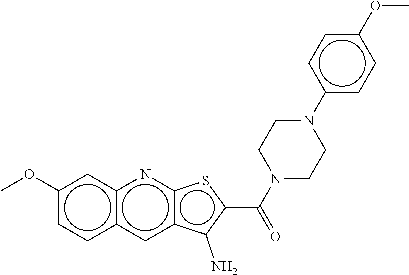

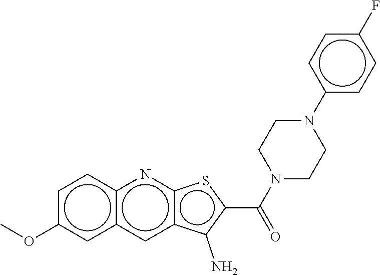

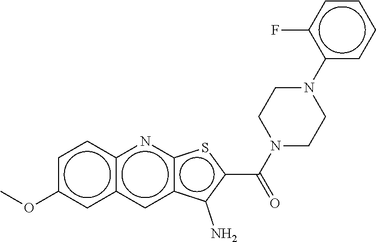

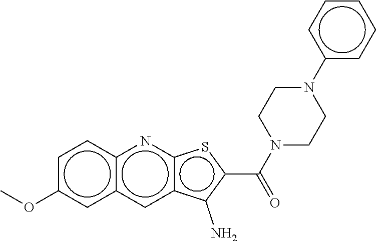

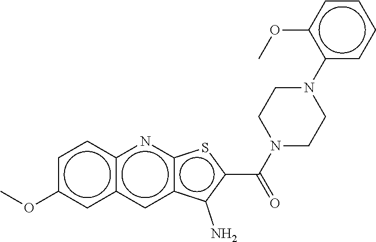

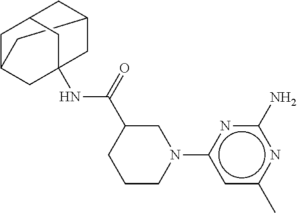

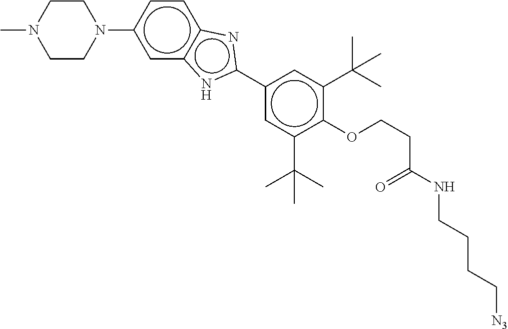

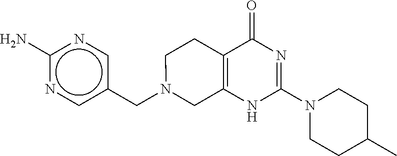

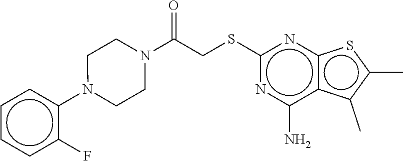

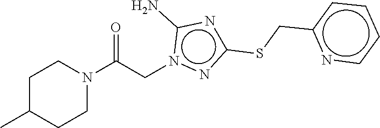

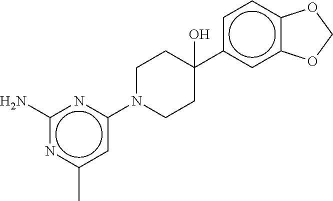

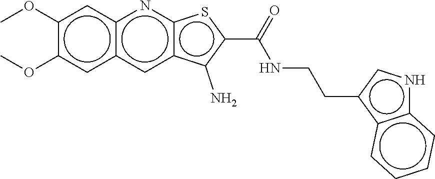

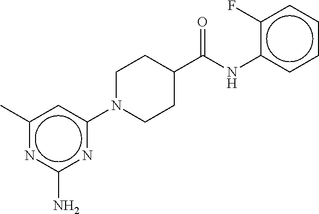

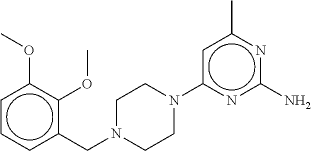

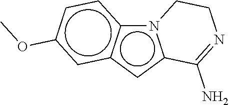

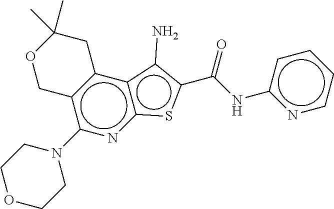

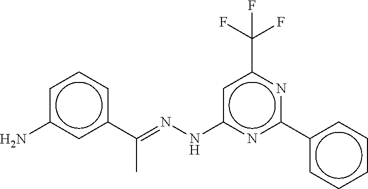

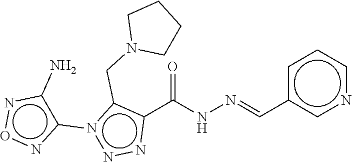

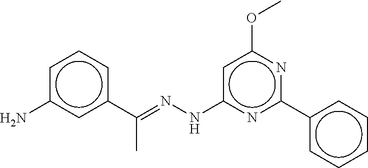

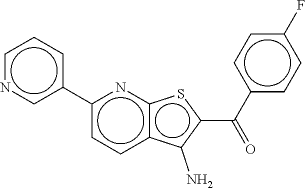

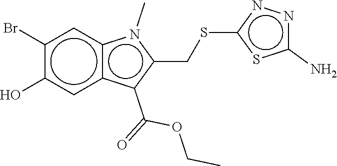

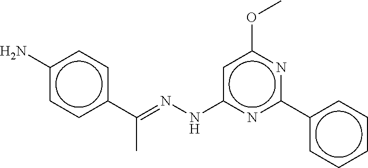

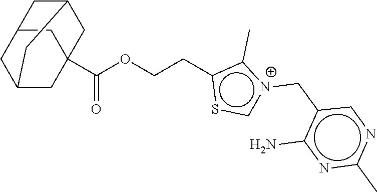

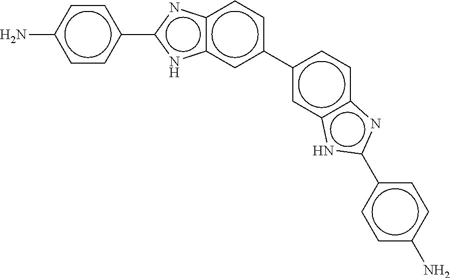

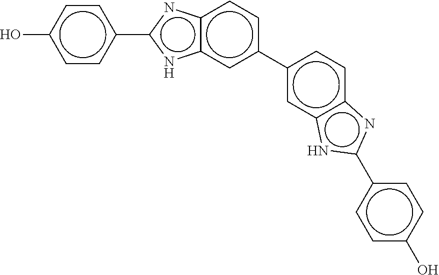

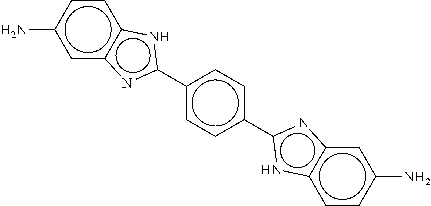

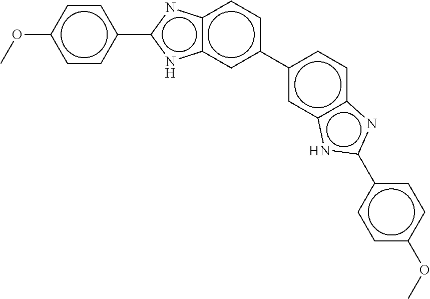

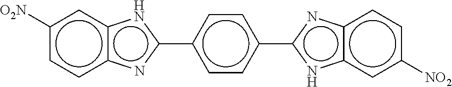

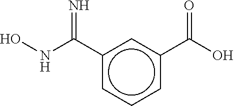

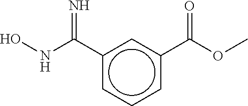

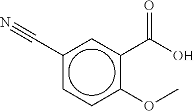

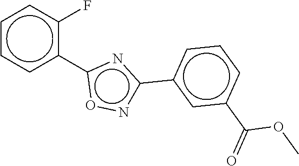

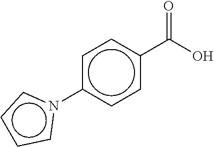

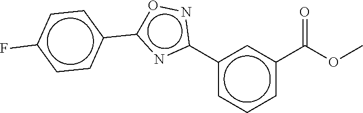

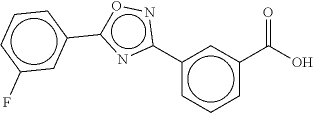

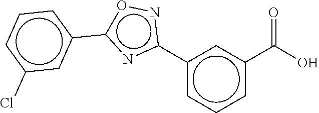

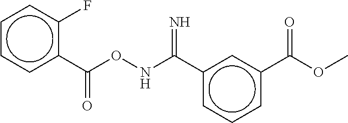

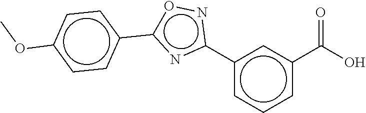

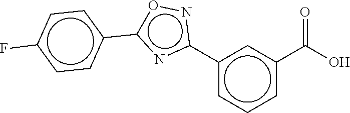

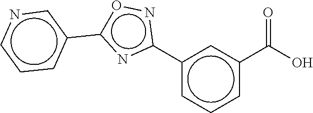

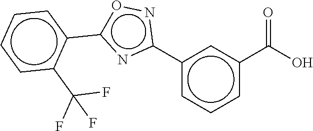

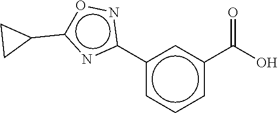

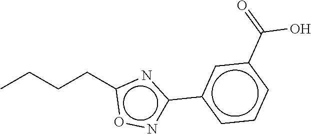

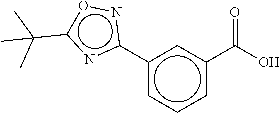

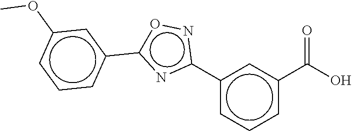

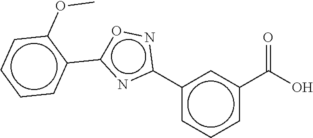

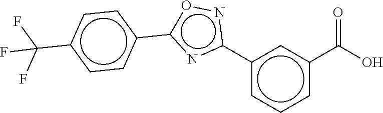

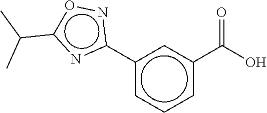

=0

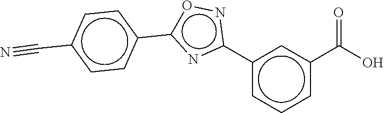

=0

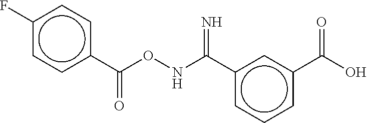

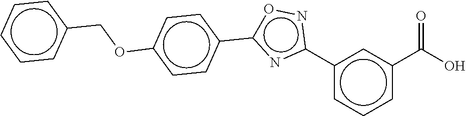

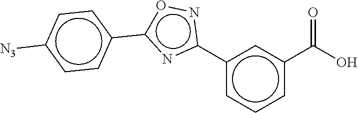

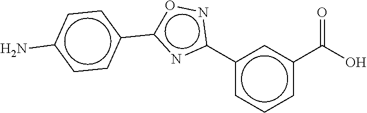

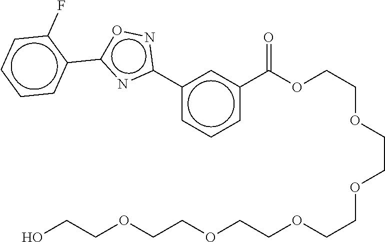

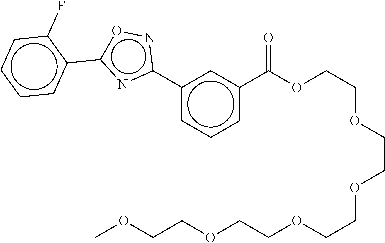

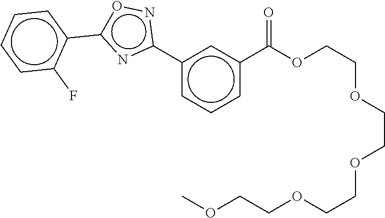

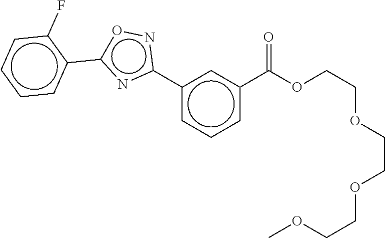

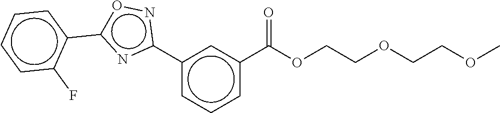

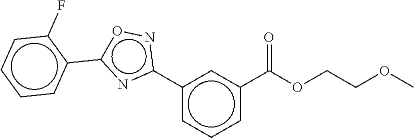

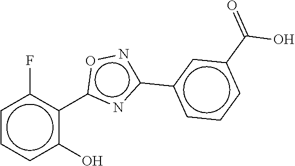

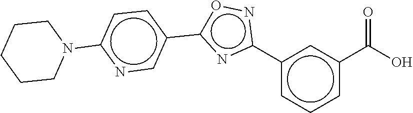

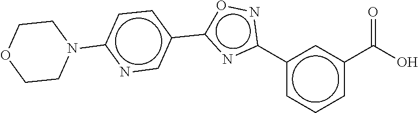

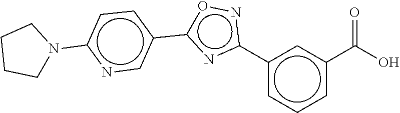

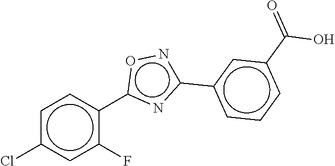

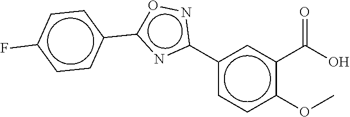

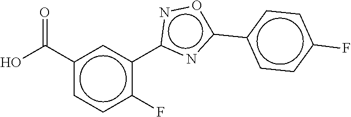

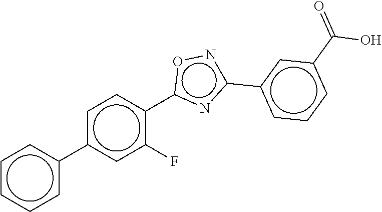

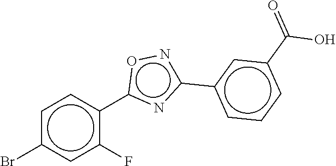

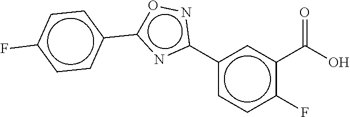

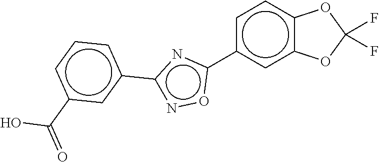

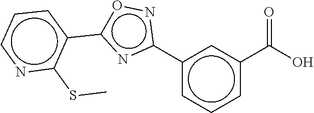

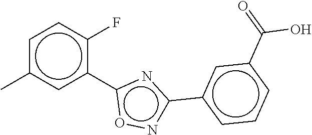

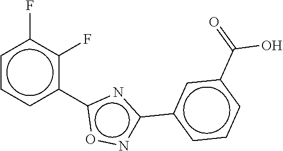

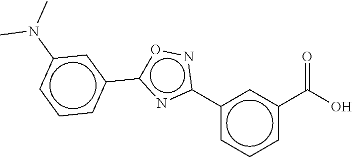

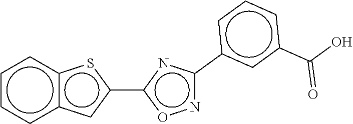

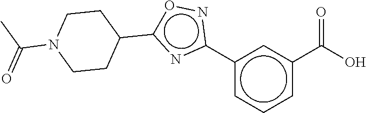

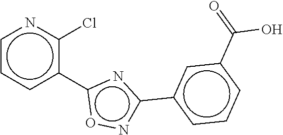

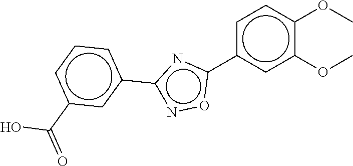

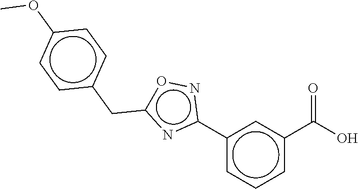

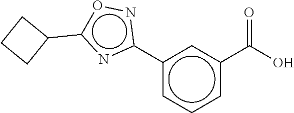

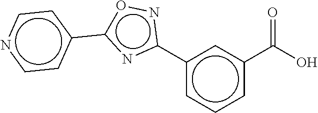

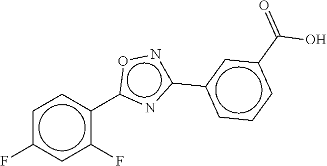

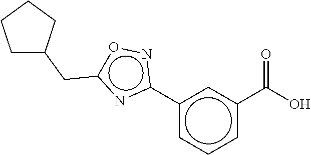

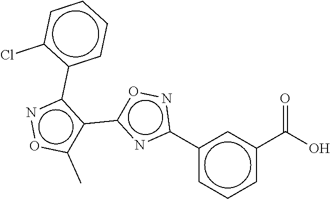

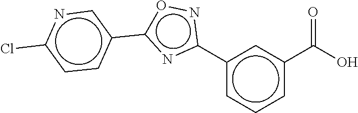

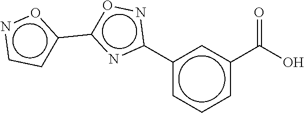

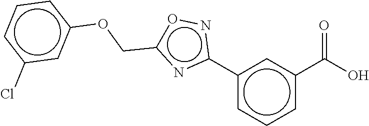

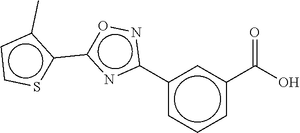

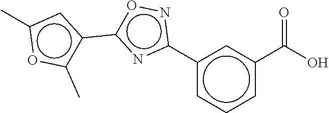

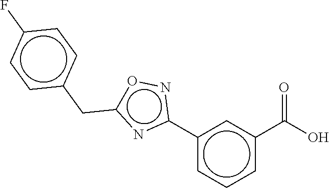

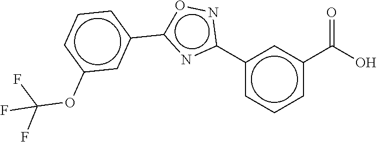

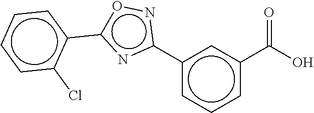

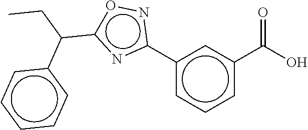

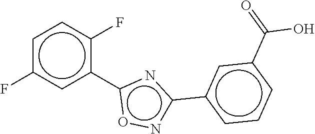

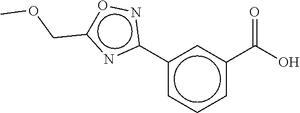

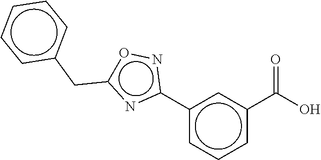

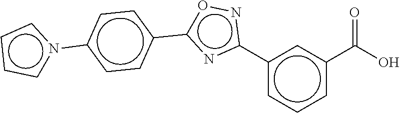

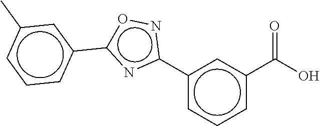

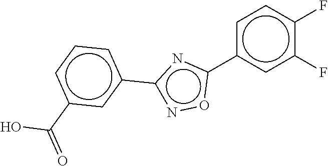

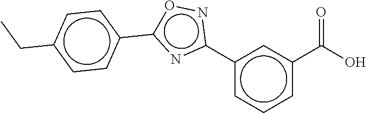

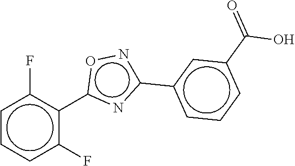

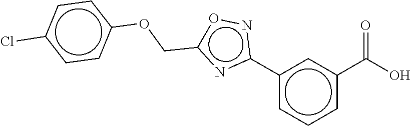

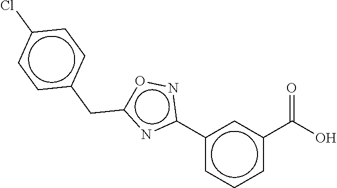

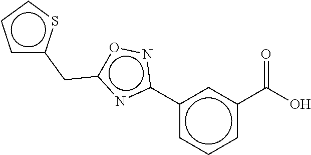

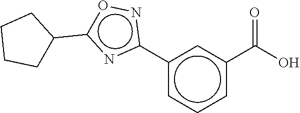

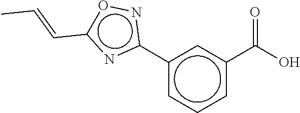

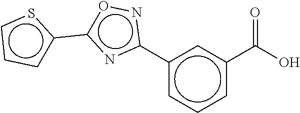

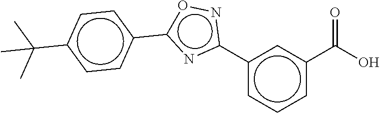

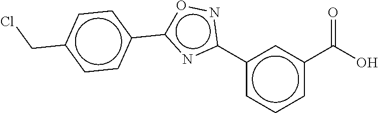

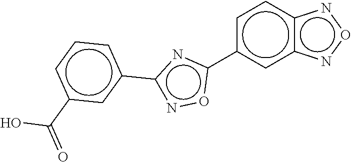

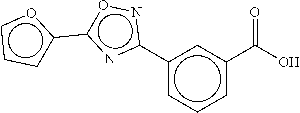

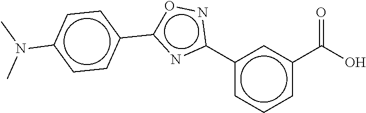

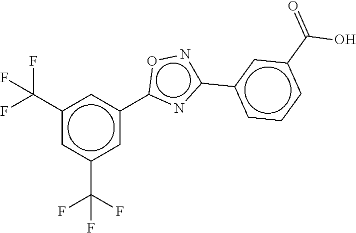

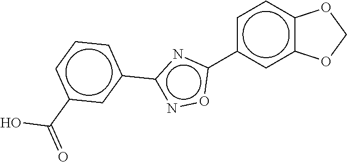

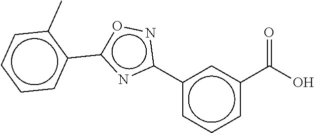

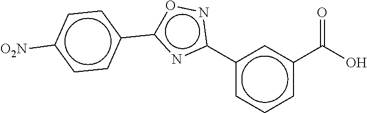

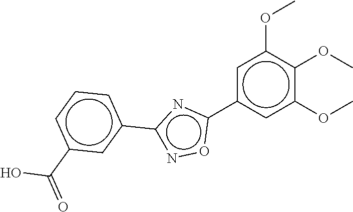

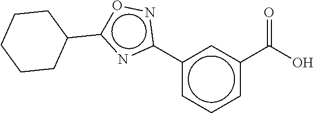

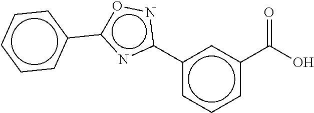

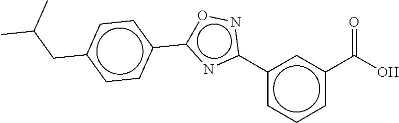

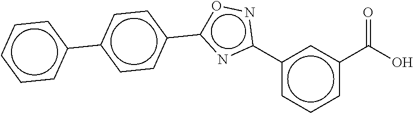

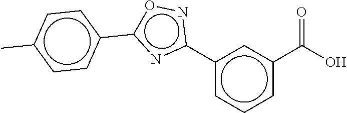

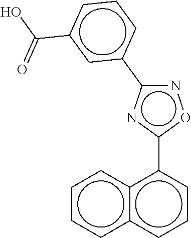

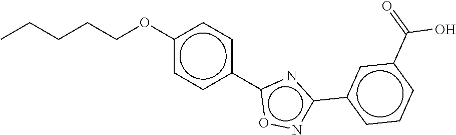

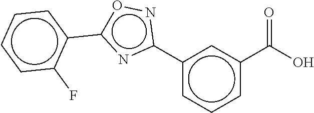

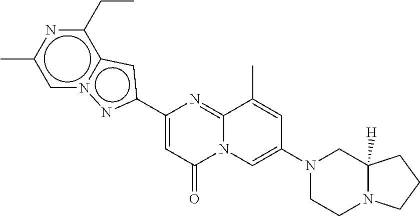

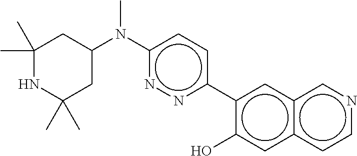

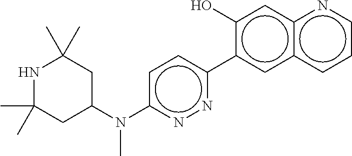

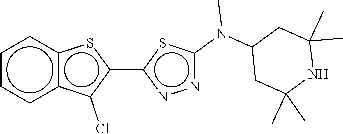

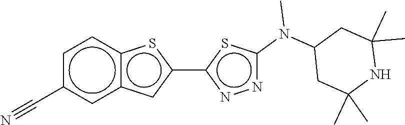

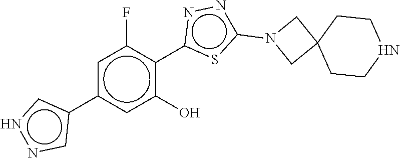

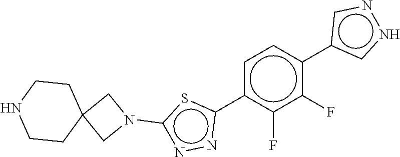

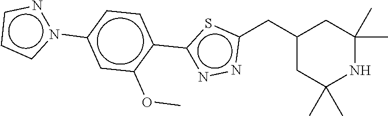

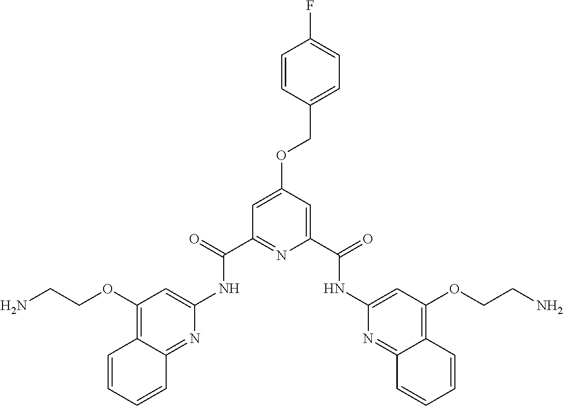 [SMILES: NCCOc1cc(NC(=O)c2cc(OCc3ccc(F)cc3)cc(C(=O)Nc3cc(OCCN)c4ccccc4n3)n2)nc2ccccc12]

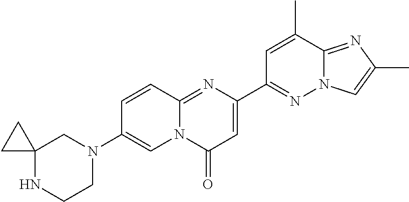 [SMILES: Cc1cn2nc(-c3cc(=O)n4cc(N5CCNC6(CC6)C5)ccc4n3)cc(C)c2n1]

1, (RG7916)
risdiplam

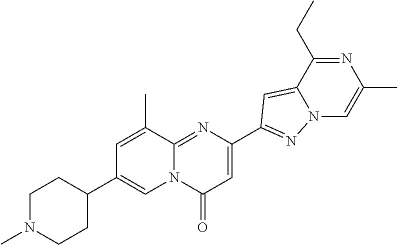

2, (RG7800)
Linezolid

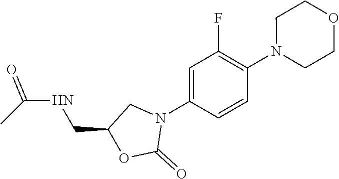 [SMILES: CC(=O)NC[C@H]1CN(c2ccc(N3CCOCC3)c(F)c2)C(=O)O1]

Warner, K. D., et al., Nat Rev Drug Discov.
2018, 17(8), 547-558; hereby incorporated by
reference.

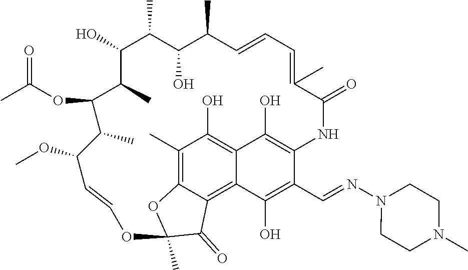 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

Warner, K. D. Nat Rev Drug Discov. 2018.

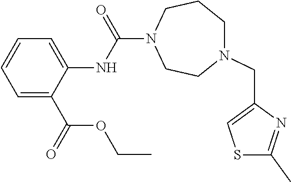 [SMILES: CCOC(=O)c1ccccc1NC(=O)N1CCCN(Cc2csc(C)n2)CC1]

Warner, K. D. Nat Rev Drug Discov. 2018.

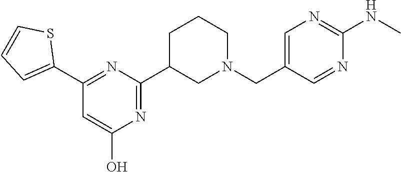 [SMILES: CNc1ncc(CN2CCCC(c3nc(O)cc(-c4cccs4)n3)C2)cn1]

=0
Warner, K. D. Nat Rev Drug Discov. 2018

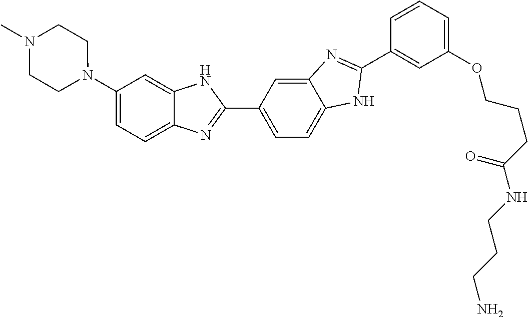 [SMILES: CN1CCN(c2ccc3nc(-c4ccc5[nH]c(-c6cccc(OCCCC(=O)NCCCN)c6)nc5c4)[nH]c3c2)CC1]

Warner, K. D. Nat Rev Drug Discov. 2018.

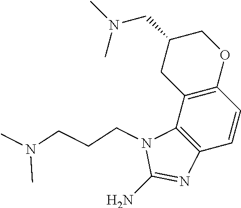 [SMILES: CN(C)CCCn1c(N)nc2ccc3c(c21)C[C@H](CN(C)C)CO3]

Warner, K. D. Nat Rev Drug Discov. 2018.

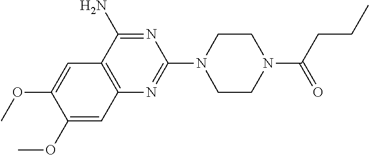 [SMILES: CCCC(=O)N1CCN(c2nc(N)c3cc(OC)c(OC)cc3n2)CC1]

Warner, K. D. Nat Rev Drug Discov. 2018.

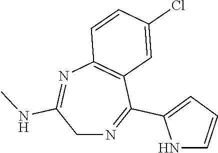 [SMILES: CNC1=Nc2ccc(Cl)cc2C(c2ccc[nH]2)=NC1]

Warner, K. D. Nat Rev Drug Discov. 2018

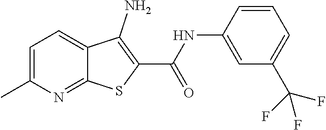 [SMILES: Cc1ccc2c(N)c(C(=O)Nc3cccc(C(F)(F)F)c3)sc2n1]

Warner, K. D. Nat Rev Drug Discov. 2018.

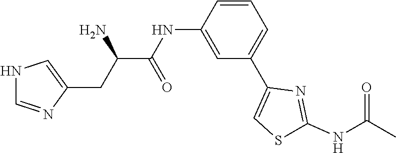 [SMILES: CC(=O)Nc1nc(-c2cccc(NC(=O)[C@H](N)Cc3c[nH]cn3)c2)cs1]

Warner, K. D. Nat Rev Drug Discov. 2018.

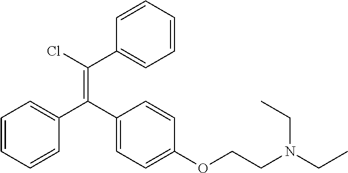 [SMILES: CCN(CC)CCOc1ccc(/C(=C(/Cl)c2ccccc2)c2ccccc2)cc1]

Warner, K. D. Nat Rev Drug Discov. 2018.

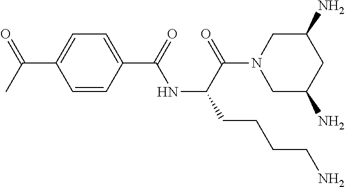 [SMILES: CC(=O)c1ccc(C(=O)N[C@@H](CCCCN)C(=O)N2C[C@H](N)C[C@H](N)C2)cc1]

Warner, K. D. Nat Rev Drug Discov. 2018.

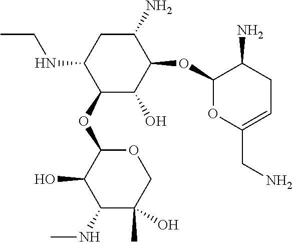 [SMILES: CCN[C@@H]1C[C@H](N)[C@@H](O[C@@H]2OC(CN)=CC[C@@H]2N)[C@H](O)[C@H]1O[C@H]1OC[C@](C)(O)[C@H](NC)[C@H]1O]

Warner, K. D. Nat Rev Drug Discov. 2018.

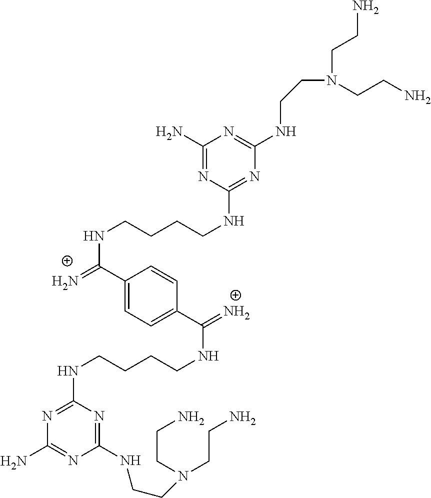 [SMILES: NCCN(CCN)CCNc1nc(N)nc(NCCCCNC(=[NH2+])c2ccc(C(=[NH2+])NCCCCNc3nc(N)nc(NCCN(CCN)CCN)n3)cc2)n1]

Warner, K. D. Nat Rev Drug Discov. 2018.

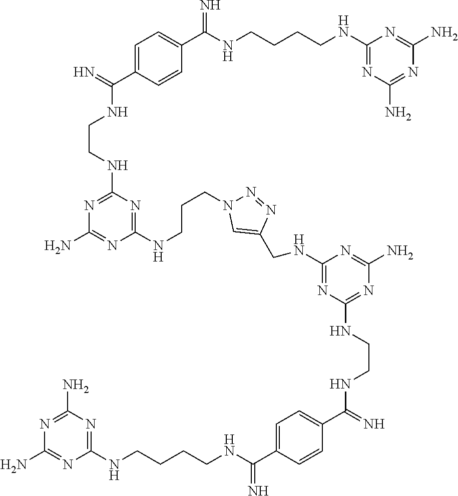 [SMILES: N=C(NCCCCNc1nc(N)nc(N)n1)c1ccc(C(=N)NCCNc2nc(N)nc(NCCCn3cc(CNc4nc(N)nc(NCCNC(=N)c5ccc(C(=N)NCCCCNc6nc(N)nc(N)n6)cc5)n4)nn3)n2)cc1]

Warner, K. D. Nat Rev Drug Discov. 2018.

### Decay Factors and RNA-Binding Proteins (RBPs)

In one aspect, the disclosure provides a composition comprising an RNA binder that binds to a target RNA transcript and a Decay Factor-recruiting Ligand (DFL), wherein the DFL binds to or recruits a decay factor.

In one aspect, the disclosure provides compositions that comprise a decay factor ligand that binds decay factors, and wherein the decay factor is a protein that binds or interacts with RNA (an RBP) and wherein the interaction of the RBP with the RNA leads to modulation of the target RNA transcript in vivo.

A decay factor as provided herein is any protein, polypeptide or biological molecule present in a cell that when brought in the proximity of a target RNA modulates that RNA. Modulating an RNA as provided herein includes, destabilizing the RNA, stabilizing the RNA, degrading the RNA, or acting on the RNA in any other capacity. Decay factors include any protein that interferes with the stability and/or activity of the RNA. In some embodiments, the decay factor is an RNA destabilizing protein, a nuclease, or an RNA-binding protein. It should be appreciated that nucleases and RNA-binding proteins are not mutually exclusive and that, for instance, some RNA-binding proteins also have nuclease activity. In some embodiments, the present disclosure provides a bifunctional compound or composition that effects recruitment to a target RNA to a nuclease capable of degrading the target RNA, or to an RNA-binding protein (RBP) that destabilizes the target RNA towards degradation by any of a cell's or tissue's mechanisms of RNA degradation.

In some embodiments, the DFL binds or attracts a complex of proteins that can degrade or otherwise modulate the RNA function (e.g., the availability for protein translation). In some embodiments, the protein complex is the CCR4-NOT (Carbon Catabolite Repression-Negative On TATA-less) complex.

### CCR4-NOT Complex

In some embodiments, the DFL binds or attracts a complex of proteins that can degrade or otherwise modulate the RNA function. In some embodiments, the DFL binds the protein complex. In some embodiments, the DFL binds one or more RBPs that are part of the protein complex. Binding of one or more RBPs is expected to bring the complete protein complex in proximity to the target RNA. In some embodiments, the DFL binds the CCR4-NOT (Carbon Catabolite Repression-Negative On TATA-less) complex, or an RBP that is a member of the CCR4-NOT complex. The CCR4-NOT complex is a large and highly conserved multifunctional assembly of proteins involved in different aspects of mRNA metabolism. Without wishing to be bound by theory, it is believed that the CCR4-NOT complex plays a role in deadenylation-dependent mRNA turnover. RBPs that are part of the CCR4-NOT complex include CNOT1, CNOT2, CNOT3, CNOT6, CNOT6L, CNOT7, CNOT8, CNOT9, CNOT10 and CNOT11. The function of the CCR4-NOT complex and each of the RBPs that make up the complex is discussed for instance in Shirai et al. Multifunctional roles of the mammalian CCR4-NOT complex in physiological phenomena, Frontiers in Genetics, 2014, 5, Article 286, which is incorporated by reference.

In some embodiments, the RBP is CNOT7.

### CNOT7

In some embodiments, the RBP is CNOT7. In some embodiments, a disclosed compound or composition comprises a small molecule CNOT7 ligand as the DFL. CNOT7 is a member of the CCR4-NOT complex. Without wishing to be bound by theory, it is believed that CNOT7 acts as exonuclease. It is thought to either directly, or in conjunction with other members of the CCR4-NOT complex, induce degradation of the target RNA (e.g., through deadenylation). CNOT7 is widely expressed in the human body.

In some embodiments, the disclosure provides compounds that bind CNOT7, but that do not bind the active site of CNOT7. In some embodiments, the disclosure provides compounds and compositions thereof, wherein the DFL binds CNOT7 without abrogating the enzymatic activity of the CNOT7 and/or the CCR4-NOT complex. By binding CNOT7 on a site other than the active site, CNOT7 will maintain its capacity to act and/or degrade RNA. Thus, the compositions provided herein, in one embodiment, can bring CNOT7 in the proximity of the target RNA, by binding both the target RNA and CNOT7, and allow the CNOT7 to act on the Target RNA (e.g., degrade it), because the CNOT7 is still functional.

In some embodiments, the compositions provided herein, by binding CNOT7, bring one more components of the CCR4-NOT complex in the proximity of the target RNA. By binding both the target RNA and CNOT7, the compounds provided herein allow the CCR4-NOT complex to act on the target RNA. The CCR4-NOT complex can act on the target RNA through any of its enzymatic capabilities. In some embodiments, the CCR4-NOT complex will act on the target RNA through the action of CNOT7, which can act on and/or degrade RNA. In some embodiments, the CCR4-NOT complex will act on the target RNA through the action of an enzymatic component other than CNOT7. In some embodiments, the CCR4-NOT complex will act on the target RNA through the action of CNOT6, which has exonuclease activity, and which can act on and/or degrade RNA.

In some embodiments, the disclosure provides compounds that bind the active site of CNOT7. By binding both the target RNA and the active site of CNOT7, the compounds provided herein allow the CCR4-NOT complex to act on the target RNA by bringing one or more components of the complex in proximity of the CNOT7. For compounds that bind the active site of CNOT7, the CCR4-NOT complex can act on the target RNA through any of its enzymatic capabilities. In some embodiments, the CCR4-NOT complex will act on the target RNA through the action of CNOT7, even if bound in the active site, which can act on and/or degrade RNA, even if the activity is suppressed as compared to CNOT7 that is not bound on the active site. In some embodiments, the CCR4-NOT complex will act on the target RNA through the action of an enzymatic component other than CNOT7. In some embodiments, the CCR4-NOT complex will act on the target RNA through the action of CNOT6, which has exonuclease activity, and which can act on and/or degrade RNA.

In any of the compositions, compounds and methods provided herein, in some embodiments, the compositions or compounds bind or interact with target RNA, and the target RNA transcript is an mRNA or a precursor, isoform, unspliced isoform, splicing intermediate, fragment, or mutant thereof. In any of the compositions, compounds and methods provided herein, in some embodiments, the compositions or compounds bind or interact with target RNA, and the target RNA transcript is selected from one of those listed in Table C or D; or a precursor, isoform, unspliced isoform, splicing intermediate, fragment, or mutant thereof. In any of the compositions, compounds and methods provided herein, in some embodiments, the compositions and compounds include an rSM that binds a target RNA. In any of the compositions, compounds and methods provided herein, in some embodiments, the rSM is selected from any one of those described in the section entitled exemplary rSMs. In any of the compositions, compounds and methods provided herein, in some embodiments, the rSM is one of those shown in Table 2.

In any of the compositions, compounds and methods provided herein, in some embodiments, the composition is a pharmaceutical composition. In any of the compositions, compounds and methods provided herein, in some embodiments, the pharmaceutical composition includes any of the compounds or compositions provided herein, or a pharmaceutically acceptable salt thereof, and a pharmaceutically acceptable carrier.

In one aspect, the present invention provides methods and compositions for the diagnosis and prognosis of cellular proliferative disorders (e.g., cancer) and the treatment of these disorders by modulating (e.g., degrading) a target RNA transcript. Cellular proliferative disorders described herein include, e.g., cancer, obesity, and proliferation-dependent diseases. Such disorders may be diagnosed using methods known in the art.

In one aspect, the present invention provides methods and compositions for the treatment of cancer by modulating (e.g. degrading) a target RNA transcript. In some embodiments, the cancer is driven or characterized by the overexpression of a protein (e.g. an oncogenic protein) and the cancer is treated by modulating (e.g. degrading) a target RNA transcript that corresponds to the overexpressed protein. In one aspect, the present invention provides methods and compositions for the treatment of cancer.

It should be appreciated that the compositions, compounds and methods provided herein allow for the modulation of the amount of target RNA and thereby the modulation of the amount of protein, or levels of protein that are expressed from the target RNA. Thus, in some embodiments, the disclosure provides compositions and compounds for methods of modifying the amount of a protein in a cell. In some embodiment, those methods include administering any of the compositions or compounds provided herein, or a pharmaceutically acceptable salt thereof, that acts on a target RNA transcript or a precursor, isoform, fragment, or mutant thereof, in an amount sufficient to modify the amount of the protein in the cell. In some embodiments, modifying the amount of a protein in a cell includes or equals reducing the amount of protein in the cell.

It should be appreciated that the compositions, compounds and methods provided herein allow for modulating the availability for protein translation of a target RNA transcript or a precursor, isoform, fragment, or mutant thereof. Thus, in some embodiments, the disclosure provides compositions and compounds for methods of modulating the availability for protein translation of a target RNA transcript or a precursor, isoform, fragment, or mutant thereof. In some embodiment, those methods include contacting the target RNA transcript or a precursor, isoform, fragment, or mutant thereof with any of the compounds or compositions provided herein or a pharmaceutically acceptable salt thereof, that binds to the target RNA transcript or an isoform, fragment, or mutant thereof.

It should be appreciated that the compositions, compounds and methods provided herein allow for modulating the translation of a target protein or mutant thereof. Thus, in some embodiments, the disclosure provides compositions and compounds for methods that include contacting a target RNA transcript or a precursor, isoform, fragment, or mutant thereof with any of the compounds or compositions provided herein or a pharmaceutically acceptable salt thereof.

It should be appreciated that the compositions, compounds and methods provided herein allow for decreasing the half-life or increasing degradation of a target RNA transcript or a precursor, isoform, fragment, or mutant thereof. Thus, in some embodiments, the disclosure provides compositions and compounds for methods that include contacting the target RNA transcript or the precursor, isoform, fragment, or mutant thereof with any of the compounds or compositions provided herein or a pharmaceutically acceptable salt thereof.

### Linkers

As defined generally above, the linker, -L¹-, in the formulae described herein is a bivalent group that connects the rSM, RNA Binder, or UBM to the ligand for the decay factor ligand (DFL). In some embodiments, e.g., for compounds of Formula I-a and embodiments thereof, -L¹- is a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or —C_{y}—; wherein one and only one of R¹, R², R³, or R⁸ is -L¹- and one end of -L¹- is covalently bound to rSM. In some embodiments, e.g., for compounds of Formula I-d and embodiments thereof, -L¹- is a covalent bond or a C₁₋₃ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-, and 1, 2, 3, 4, 5, 6, or 7 methylene units are optionally replaced with —OCH₂CH₂— or —CH₂CH₂O—; wherein one and only one of R¹, R², R³, R⁸, or R¹¹ is -L¹- and one end of -L¹- is covalently bound to rSM. In some embodiments, -L¹- is a covalent bond or a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of -L¹- are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(N(R)₂)—, an amino acid,

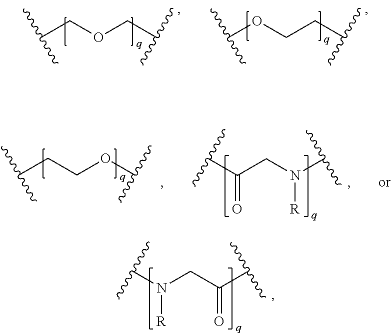

wherein:
each -Cy²- is independently an optionally substituted bivalent ring selected from phenylenyl, an 8-12 membered bicyclic arylenyl, a 3-8 membered saturated or partially unsaturated carbocyclylenyl, an 8-12 membered bicyclic saturated or partially unsaturated carbocyclylenyl, a 3-8 membered saturated or partially unsaturated heterocyclylenyl having 1-3 heteroatoms independently selected from nitrogen, oxygen, and sulfur, an 8-12 membered bicyclic saturated or partially unsaturated heterocyclylenyl having 1-5 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
wherein each q is independently 1, 2, or 3.

In some embodiments, -L¹- is a covalent bond. In some embodiments, -L¹- is a bivalent, saturated or unsaturated, straight or branched, optionally substituted C₁₋₅₀ hydrocarbon chain, wherein 0, 1, 2, 3, 4, 5, 6, 7, 8, 9, or 10 methylene units of -L¹- are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(N(R)₂)—, an amino acid,

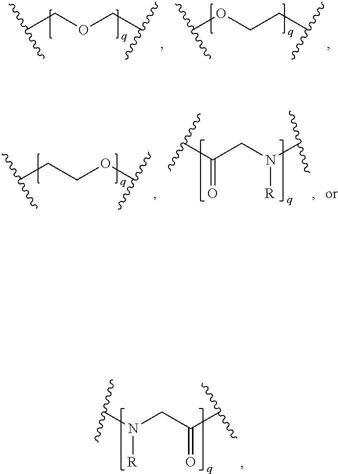

In some embodiments, -L¹- is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, 8, 9, or 10 methylene units of L¹ are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(N(R)₂)—, an amino acid,

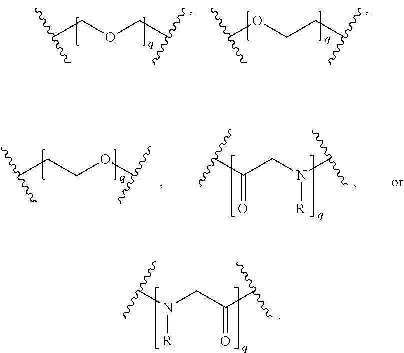

In some embodiments, -L¹- is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, 8, 9, or 10 methylene units of -L¹- are independently replaced by -Cy²-, —O—, —N(R)—, —S—, —OC(O)—, —C(O)O—, —C(O)—, —C(S)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, —N(R)C(O)N(R)—, —N(R)C(S)N(R)—, —Si(R)₂—, —Si(OH)(R)—, —Si(OH)₂—, —P(O)(OR)—, —P(O)(R)—, —P(O)(NR₂)—, an amino acid,

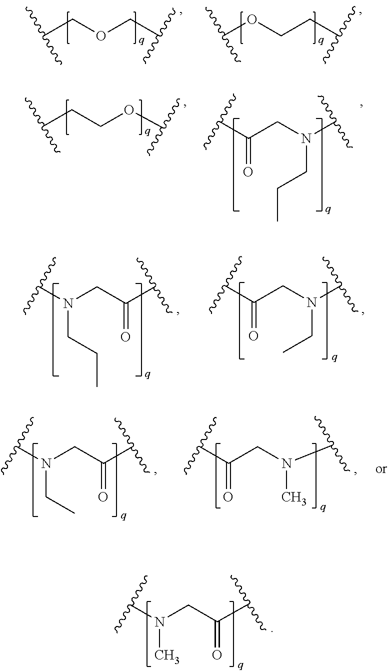

In some embodiments, -L¹- is a bivalent, saturated or unsaturated, straight or branched, optionally substituted C₁₋₅₀, C₁₋₄₀, C₁₋₃₀, C₁₋₂₀, C₁₋₁₅, C₁₋₁₀, C₁₋₅, C₂₋₅₀, C₂₋₄₀, C₂₋₃₀, C₂₋₂₀, C₂₋₁₅, C₂₋₁₀, C₃₋₅₀, C₃₋₄₀, C₃₋₃₀, C₃₋₂₀, C₃₋₁₅, C₃₋₁₀, C₄₋₅₀, C₄₋₄₀, C₄₋₃₀, C₄₋₂₀, C₄₋₁₅, C₄₋₁₀, C₅₋₅₀, C₅₋₄₀, C₅₋₃₀, C₅₋₂₀, C₅₋₁₅, C₅₋₁₀, C₆₋₅₀, C₆₋₄₀, C₆₋₃₀, C₆₋₂₀, C₆₋₁₅, C₇₋₅₀, C₇₋₄₀, C₇₋₃₀, C₇₋₂₀, C₇₋₁₅, C₈₋₅₀, C₈₋₄₀, C₈₋₃₀, C₈₋₂₀, C₈₋₁₅, C₁₀₋₅₀, C₁₀₋₄₀, C₁₀₋₃₀, C₁₀₋₂₀, C₁₀₋₁₅, C₁₂₋₅₀, C₁₂₋₄₀, C₁₂₋₃₀, C₁₂₋₂₀, C₁₅₋₅₀, C₁₅₋₄₀, C₁₅₋₃₀, C₁₅₋₂₀, C₂₀₋₅₀, C₂₀₋₄₀, or C₂₀₋₃₀ hydrocarbon chain, wherein 0, 1, 2, 3, 4, 5, 6, 7, or 8 methylene units of -L¹- are independently replaced by -Cy²-, —O—, —N(R)—, —S—, —OC(O)—, —C(O)O—, —C(O)—, —C(S)—, —S(O)—, —S(O)₂—, —N(R)C(O)—, —C(O)N(R)—, —OC(O)N(R)—, —N(R)C(O)O—, an amino acid,

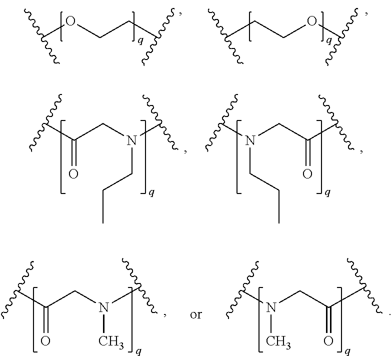

In some embodiments, -L¹- comprises 1, 2, 3, 4, 5, or 6 PEG units,

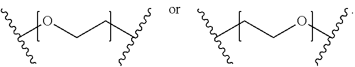

In some embodiments, -L¹- comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 PEG units. In some embodiments, L¹ comprises 1, 2, 3, 4, 5, 6, 7, 8, 9, 10, 11, or 12 units; or 1, 2 3, 4, 5, or 6 units, of

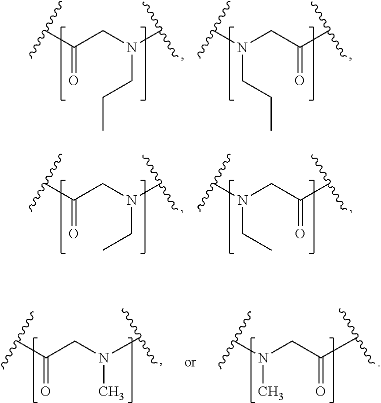

In some embodiments, -L¹- is a saturated chain. In some embodiments, -L¹- comprises at least one unsaturated pair of carbon atoms, i.e., at least one double or triple carbon-carbon bond. In some embodiments, -L¹- comprises 1, 2, 3, 4, or 5 double or triple carbon-carbon bonds. In some embodiments, -L¹- is a straight hydrocarbon chain wherein methylene units of -L¹- are optionally replaced or substituted as described above. In some embodiments, -L¹- is a saturated, straight hydrocarbon chain wherein methylene units of -L¹- are optionally replaced or substituted as described above.

In some embodiments, -L¹- is substituted with 1, 2, 3, 4, 5, 6, 7, 8, 9, or 10 “optional substituents” as defined herein. In some embodiments, each substituent is independently selected from deuterium, halogen, —CN, —OR, —N(R)₂, —SR, a 3-8 membered saturated or partially unsaturated monocyclic carbocyclic ring, phenyl optionally substituted with one or more C₁₋₄ alkyl, —CO₂R, —OR, —CON(R)₂, —N(R)₂, or halogen, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, an 8-10 membered bicyclic heteroaromatic ring having 1-5 heteroatoms independently selected from nitrogen, oxygen, or sulfur, or a C₁₋₆ aliphatic group optionally substituted with —CN, —OR, —N(R)₂, —SR, a 3-8 membered saturated or partially unsaturated monocyclic carbocyclic ring, phenyl optionally substituted with one or more C₁₋₄ alkyl, —CO₂R, —OR, —CON(R)₂, —N(R)₂, or halogen, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur, or the C₁₋₆ aliphatic is optionally substituted with 1, 2, 3, 4, 5, or 6 deuterium or halogen atoms; or two substituents attached to the same carbon atom, taken together with the carbon atom to which they are attached, form a 3-6 membered saturated monocyclic carbocyclic ring or 3-6 membered saturated heterocyclic ring having 1-2 heteroatoms independently selected from nitrogen, oxygen, or sulfur.

As described above, in some embodiments, a methylene unit of L¹ is replaced with an amino acid. The amino acid may be naturally-occurring or non-naturally occurring. In some embodiments, the amino acid is selected from a non-polar or branched chain amino acid (BCAA). In some embodiments, the amino acid is selected from valine, isoleucine, leucine, methionine, alanine, proline, glycine, phenylalanine, tyrosine, tryptophan, histidine, asparagine, glutamine, serine threonine, lysine, arginine, histidine, aspartic acid, glutamic acid, cysteine, selenocysteine, or tyrosine. In some embodiments, the amino acid is an L-amino acid. In some embodiments, the amino acid is a D-amino acid.

In some embodiments, -L¹- is selected from one of those depicted in Table 3, below.

TABLE 3
Exemplary Linkers

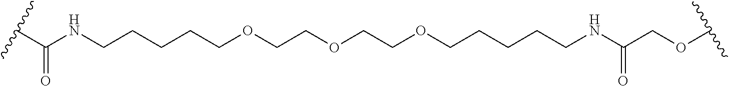 [SMILES: *OCC(=O)NCCCCCOCCOCCOCCCCCNC(*)=O]

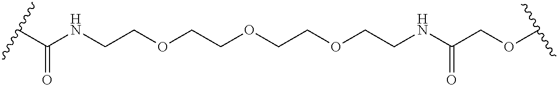 [SMILES: *OCC(=O)NCCOCCOCCOCCNC(*)=O]

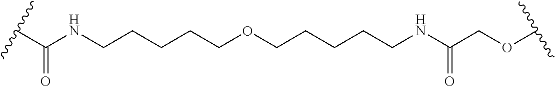 [SMILES: *OCC(=O)NCCCCCOCCCCCNC(*)=O]

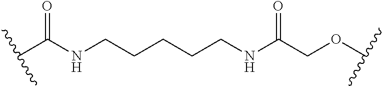 [SMILES: *OCC(=O)NCCCCCNC(*)=O]

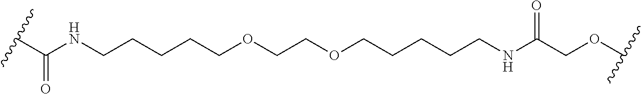 [SMILES: *OCC(=O)NCCCCCOCCOCCCCCNC(*)=O]

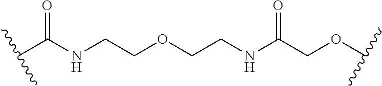 [SMILES: *OCC(=O)NCCOCCNC(*)=O]

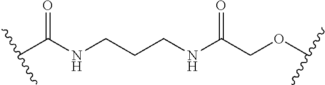 [SMILES: *OCC(=O)NCCCNC(*)=O]

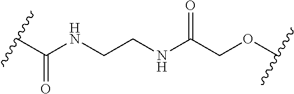 [SMILES: *OCC(=O)NCCNC(*)=O]

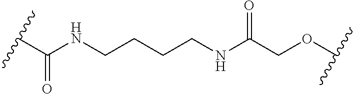 [SMILES: *OCC(=O)NCCCCNC(*)=O]

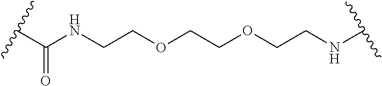 [SMILES: *NCCOCCOCCNC(*)=O]

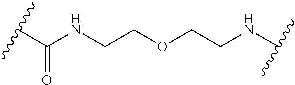 [SMILES: *NCCOCCNC(*)=O]

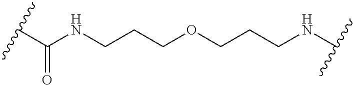 [SMILES: *NCCCOCCCNC(*)=O]

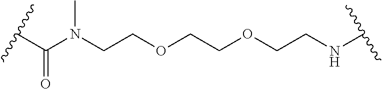 [SMILES: *NCCOCCOCCN(C)C(*)=O]

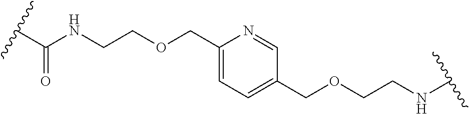 [SMILES: *NCCOCc1ccc(COCCNC(*)=O)nc1]

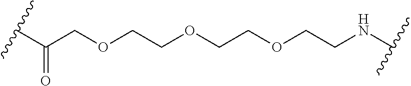 [SMILES: *NCCOCCOCCOCC(*)=O]

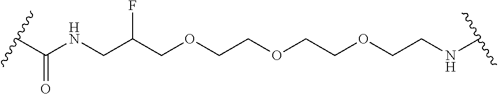 [SMILES: *NCCOCCOCCOCC(F)CNC(*)=O]

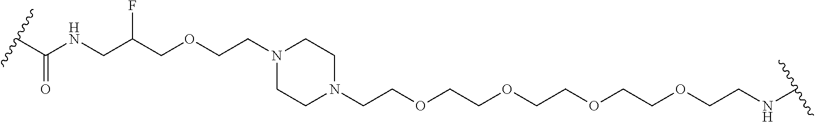 [SMILES: *NCCOCCOCCOCCOCCN1CCN(CCOCC(F)CNC(*)=O)CC1]

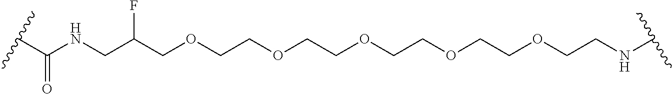 [SMILES: *NCCOCCOCCOCCOCCOCC(F)CNC(*)=O]

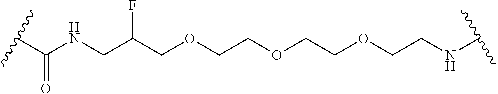 [SMILES: *NCCOCCOCCOCC(F)CNC(*)=O]

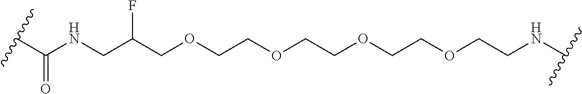 [SMILES: *NCCOCCOCCOCCOCC(F)CNC(*)=O]

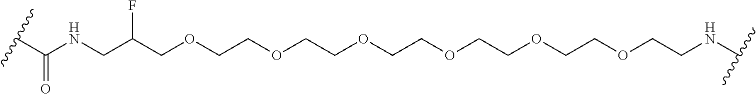 [SMILES: *NCCOCCOCCOCCOCCOCCOCC(F)CNC(*)=O]

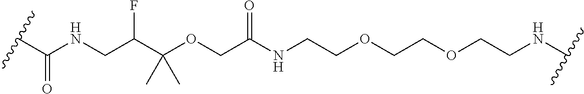 [SMILES: *NCCOCCOCCNC(=O)COC(C)(C)C(F)CNC(*)=O]

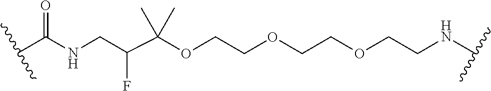 [SMILES: *NCCOCCOCCOC(C)(C)C(F)CNC(*)=O]

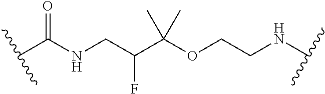 [SMILES: *NCCOC(C)(C)C(F)CNC(*)=O]

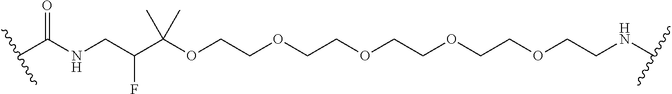 [SMILES: *NCCOCCOCCOCCOCCOC(C)(C)C(F)CNC(*)=O]

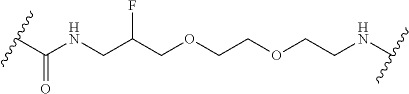 [SMILES: *NCCOCCOCC(F)CNC(*)=O]

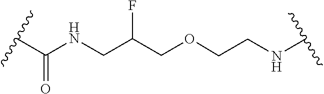 [SMILES: *NCCOCC(F)CNC(*)=O]

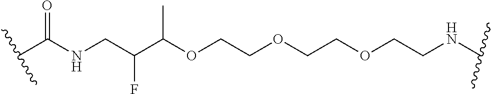 [SMILES: *NCCOCCOCCOC(C)C(F)CNC(*)=O]

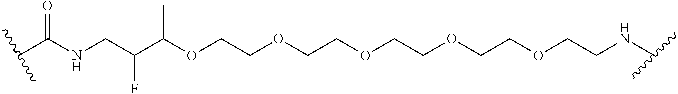 [SMILES: *NCCOCCOCCOCCOCCOC(C)C(F)CNC(*)=O]

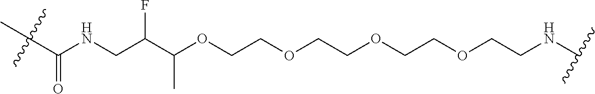 [SMILES: *NCCOCCOCCOCCOC(C)C(F)CNC(*)=O]

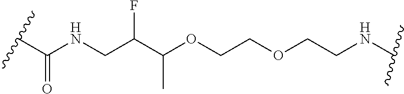 [SMILES: *NCCOCCOC(C)C(F)CNC(*)=O]

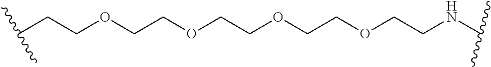 [SMILES: *CCOCCOCCOCCOCCN*]

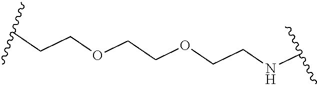 [SMILES: *CCOCCOCCN*]

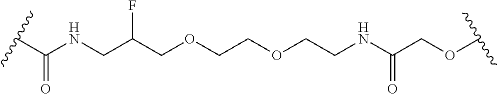 [SMILES: *OCC(=O)NCCOCCOCC(F)CNC(*)=O]

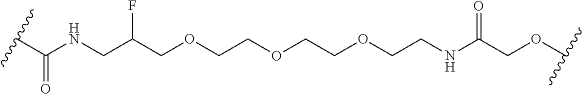 [SMILES: *OCC(=O)NCCOCCOCCOCC(F)CNC(*)=O]

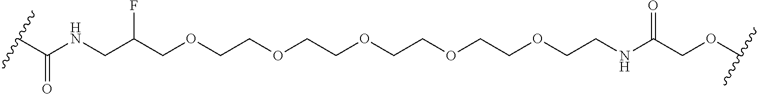 [SMILES: *OCC(=O)NCCOCCOCCOCCOCCOCC(F)CNC(*)=O]

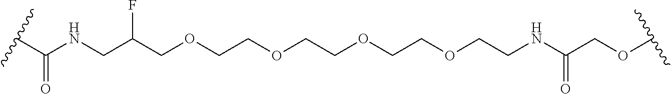 [SMILES: *OCC(=O)NCCOCCOCCOCCOCC(F)CNC(*)=O]

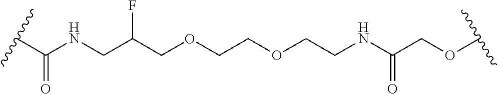 [SMILES: *OCC(=O)NCCOCCOCC(F)CNC(*)=O]

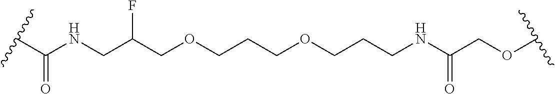 [SMILES: *OCC(=O)NCCCOCCCOCC(F)CNC(*)=O]

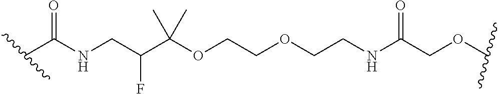 [SMILES: *OCC(=O)NCCOCCOC(C)(C)C(F)CNC(*)=O]

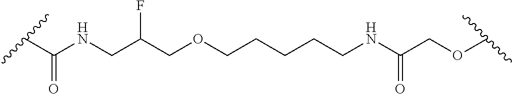 [SMILES: *OCC(=O)NCCCCCOCC(F)CNC(*)=O]

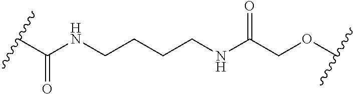 [SMILES: *OCC(=O)NCCCCNC(*)=O]

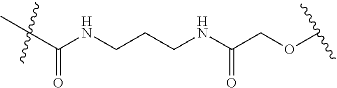 [SMILES: *OCC(=O)NCCCNC(*)=O]

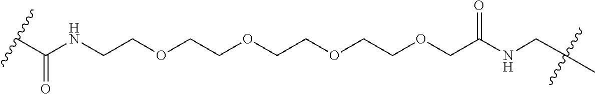 [SMILES: *CNC(=O)COCCOCCOCCOCCNC(*)=O]

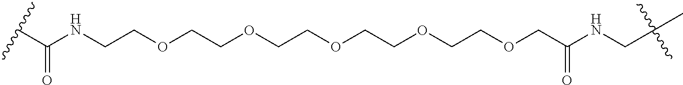 [SMILES: *CNC(=O)COCCOCCOCCOCCOCCNC(*)=O]

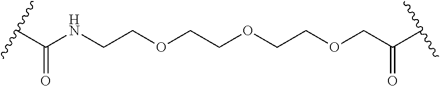 [SMILES: *C(=O)COCCOCCOCCNC(*)=O]

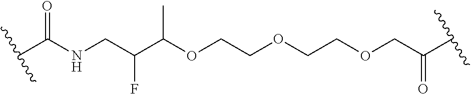 [SMILES: *C(=O)COCCOCCOC(C)C(F)CNC(*)=O]

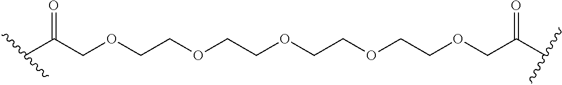 [SMILES: *C(=O)COCCOCCOCCOCCOCC(*)=O]

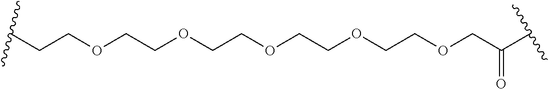 [SMILES: *CCOCCOCCOCCOCCOCC(*)=O]

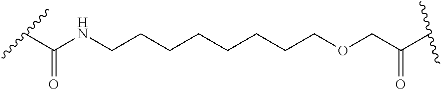 [SMILES: *C(=O)COCCCCCCCCNC(*)=O]

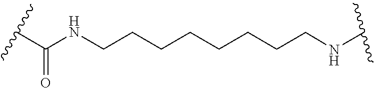 [SMILES: *NCCCCCCCCNC(*)=O]

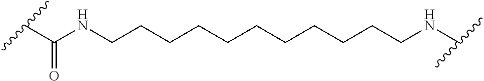 [SMILES: *NCCCCCCCCCCCNC(*)=O]

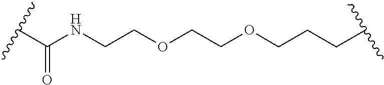 [SMILES: *CCCOCCOCCNC(*)=O]

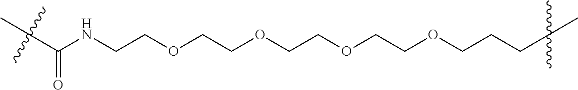 [SMILES: *CCCOCCOCCOCCOCCNC(*)=O]

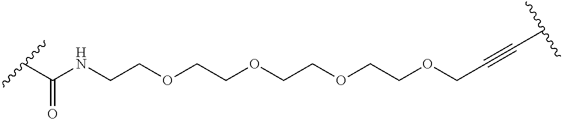 [SMILES: *C#CCOCCOCCOCCOCCNC(*)=O]

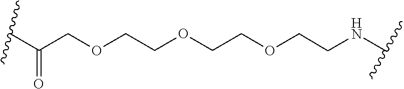 [SMILES: *NCCOCCOCCOCC(*)=O]

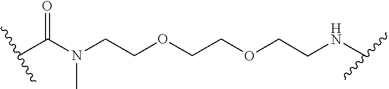 [SMILES: *NCCOCCOCCN(C)C(*)=O]

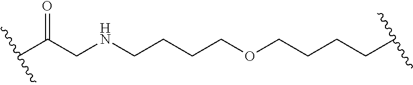 [SMILES: *CCCCOCCCCNCC(*)=O]

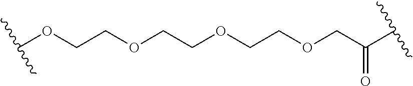 [SMILES: *OCCOCCOCCOCC(*)=O]

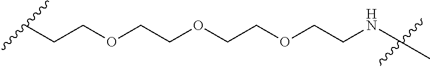 [SMILES: *CCOCCOCCOCCN*]

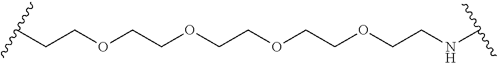 [SMILES: *CCOCCOCCOCCOCCN*]

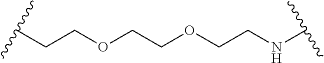 [SMILES: *CCOCCOCCN*]

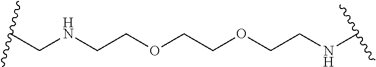 [SMILES: *CNCCOCCOCCN*]

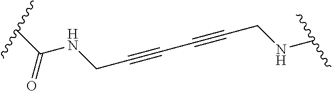 [SMILES: *NCC#CC#CCNC(*)=O]

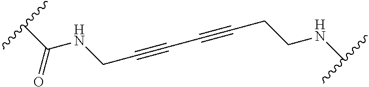 [SMILES: *NCCC#CC#CCNC(*)=O]

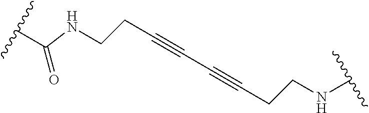 [SMILES: *NCCC#CC#CCCNC(*)=O]

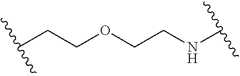 [SMILES: *CCOCCN*]

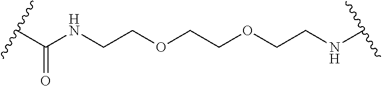 [SMILES: *NCCOCCOCCNC(*)=O]

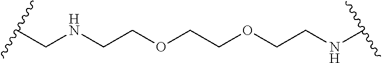 [SMILES: *CNCCOCCOCCN*]

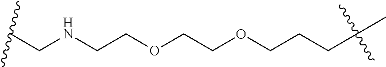 [SMILES: *CCCOCCOCCNC*]

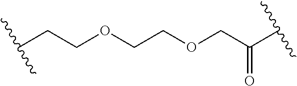 [SMILES: *CCOCCOCC(*)=O]

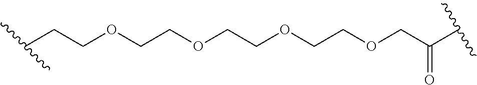 [SMILES: *CCOCCOCCOCCOCC(*)=O]

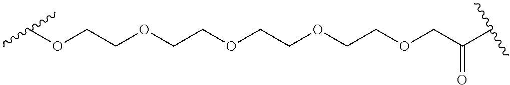 [SMILES: *OCCOCCOCCOCCOCC(*)=O]

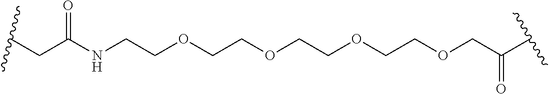 [SMILES: *CC(=O)NCCOCCOCCOCCOCC(*)=O]

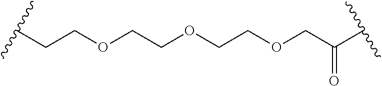 [SMILES: *CCOCCOCCOCC(*)=O]

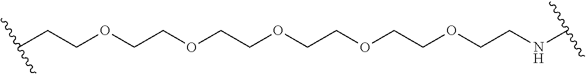 [SMILES: *CCOCCOCCOCCOCCOCCN*]

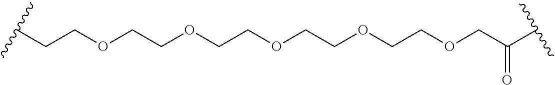 [SMILES: *CCOCCOCCOCCOCCOCC(*)=O]

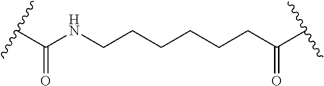 [SMILES: *C(=O)CCCCCCNC(*)=O]

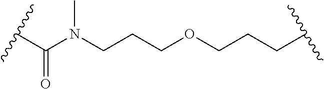 [SMILES: *CCCOCCCN(C)C(*)=O]

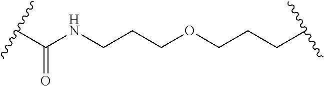 [SMILES: *CCCOCCCNC(*)=O]

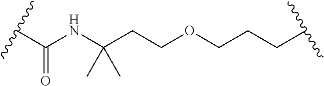 [SMILES: *CCCOCCC(C)(C)NC(*)=O]

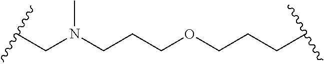 [SMILES: *CCCOCCCN(C)C*]

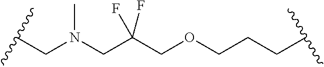 [SMILES: *CCCOCC(F)(F)CN(C)C*]

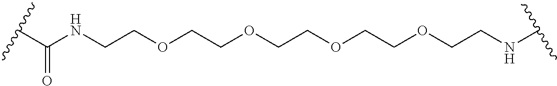 [SMILES: *NCCOCCOCCOCCOCCNC(*)=O]

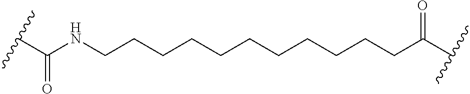 [SMILES: *C(=O)CCCCCCCCCCCNC(*)=O]

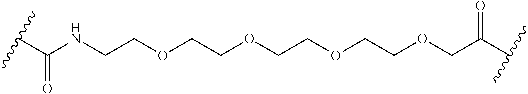 [SMILES: *C(=O)COCCOCCOCCOCCNC(*)=O]

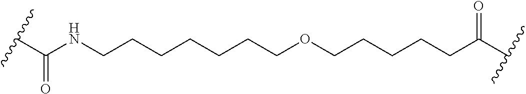 [SMILES: *C(=O)CCCCCOCCCCCCCNC(*)=O]

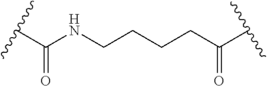 [SMILES: *C(=O)CCCCNC(*)=O]

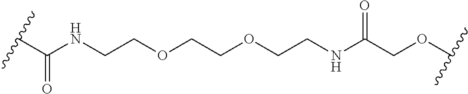 [SMILES: *OCC(=O)NCCOCCOCCNC(*)=O]

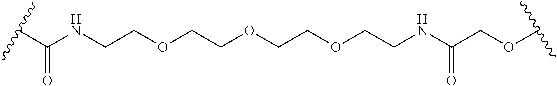 [SMILES: *OCC(=O)NCCOCCOCCOCCNC(*)=O]

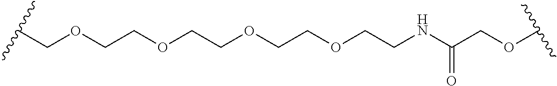 [SMILES: *COCCOCCOCCOCCNC(=O)CO*]

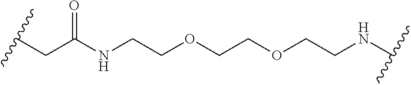 [SMILES: *CC(=O)NCCOCCOCCN*]

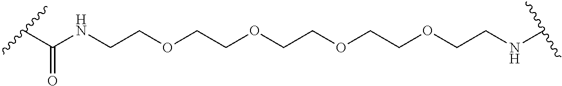 [SMILES: *NCCOCCOCCOCCOCCNC(*)=O]

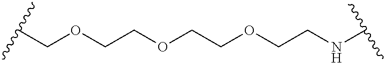 [SMILES: *COCCOCCOCCN*]

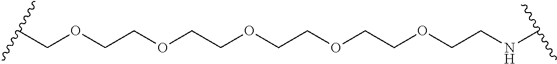 [SMILES: *COCCOCCOCCOCCOCCN*]

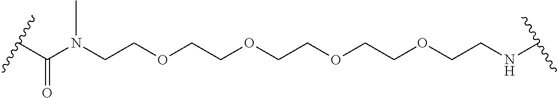 [SMILES: *NCCOCCOCCOCCOCCN(C)C(*)=O]

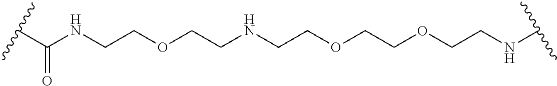 [SMILES: *NCCOCCOCCNCCOCCNC(*)=O]

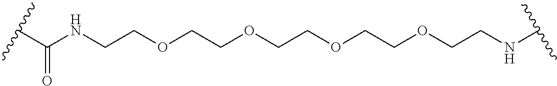 [SMILES: *NCCOCCOCCOCCOCCNC(*)=O]

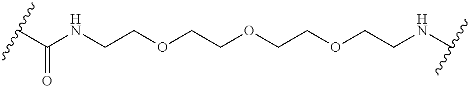 [SMILES: *NCCOCCOCCOCCNC(*)=O]

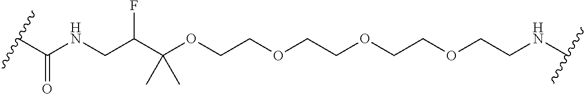 [SMILES: *NCCOCCOCCOCCOC(C)(C)C(F)CNC(*)=O]

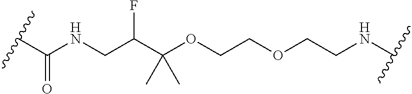 [SMILES: *NCCOCCOC(C)(C)C(F)CNC(*)=O]

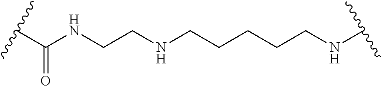 [SMILES: *NCCCCCNCCNC(*)=O]

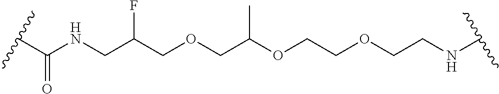 [SMILES: *NCCOCCOC(C)COCC(F)CNC(*)=O]

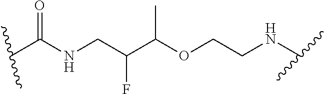 [SMILES: *NCCOC(C)C(F)CNC(*)=O]

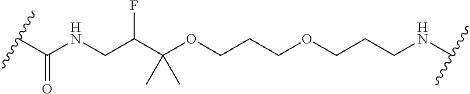 [SMILES: *NCCCOCCCOC(C)(C)C(F)CNC(*)=O]

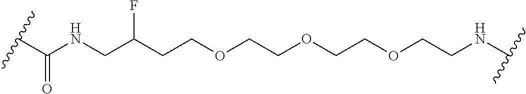 [SMILES: *NCCOCCOCCOCCC(F)CNC(*)=O]

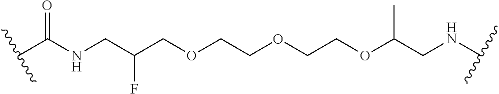 [SMILES: *NCC(C)OCCOCCOCC(F)CNC(*)=O]

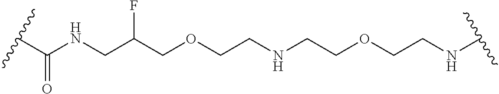 [SMILES: *NCCOCCNCCOCC(F)CNC(*)=O]

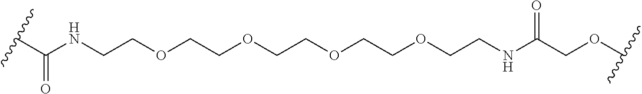 [SMILES: *OCC(=O)NCCOCCOCCOCCOCCNC(*)=O]

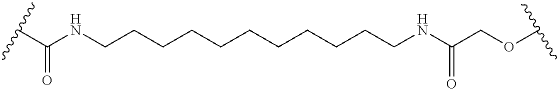 [SMILES: *OCC(=O)NCCCCCCCCCCCNC(*)=O]

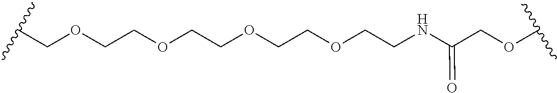 [SMILES: *COCCOCCOCCOCCNC(=O)CO*]

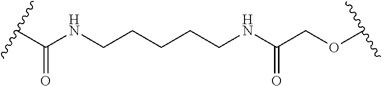 [SMILES: *OCC(=O)NCCCCCNC(*)=O]

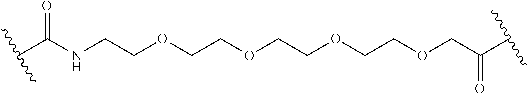 [SMILES: *C(=O)COCCOCCOCCOCCNC(*)=O]

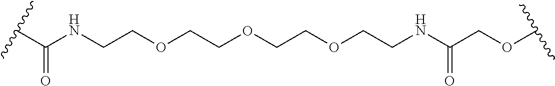 [SMILES: *OCC(=O)NCCOCCOCCOCCNC(*)=O]

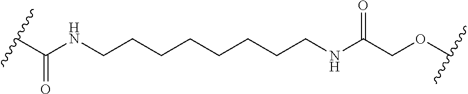 [SMILES: *OCC(=O)NCCCCCCCCNC(*)=O]

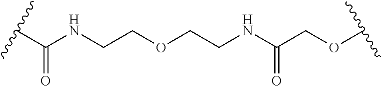 [SMILES: *OCC(=O)NCCOCCNC(*)=O]

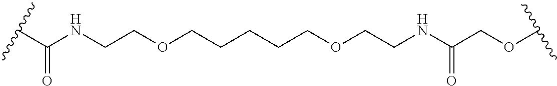 [SMILES: *OCC(=O)NCCOCCCCCOCCNC(*)=O]

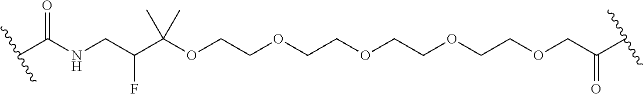 [SMILES: *C(=O)COCCOCCOCCOCCOC(C)(C)C(F)CNC(*)=O]

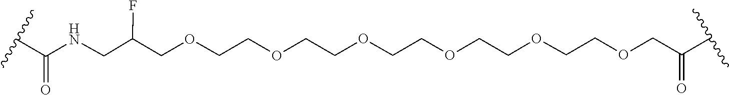 [SMILES: *C(=O)COCCOCCOCCOCCOCCOCC(F)CNC(*)=O]

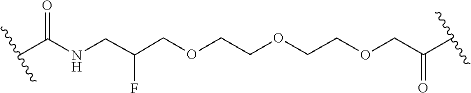 [SMILES: *C(=O)COCCOCCOCC(F)CNC(*)=O]

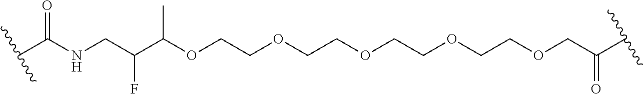 [SMILES: *C(=O)COCCOCCOCCOCCOC(C)C(F)CNC(*)=O]

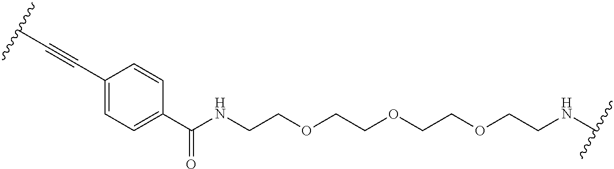 [SMILES: *C#Cc1ccc(C(=O)NCCOCCOCCOCCN*)cc1]

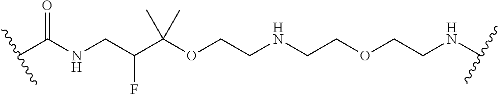 [SMILES: *NCCOCCNCCOC(C)(C)C(F)CNC(*)=O]

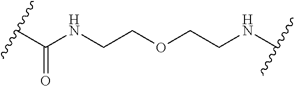 [SMILES: *NCCOCCNC(*)=O]

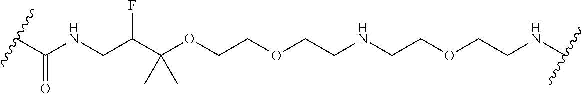 [SMILES: *NCCOCCNCCOCCOC(C)(C)C(F)CNC(*)=O]

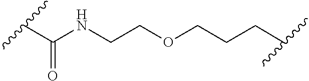 [SMILES: *CCCOCCNC(*)=O]

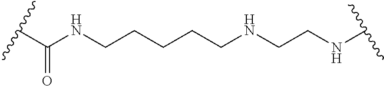 [SMILES: *NCCNCCCCCNC(*)=O]

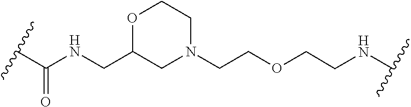 [SMILES: *NCCOCCN1CCOC(CNC(*)=O)C1]

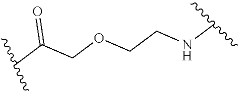 [SMILES: *NCCOCC(*)=O]

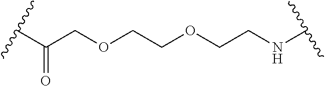 [SMILES: *NCCOCCOCC(*)=O]

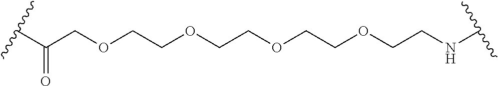 [SMILES: *NCCOCCOCCOCCOCC(*)=O]

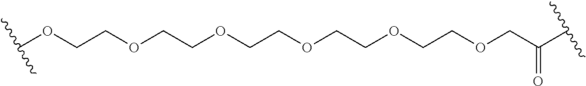 [SMILES: *OCCOCCOCCOCCOCCOCC(*)=O]

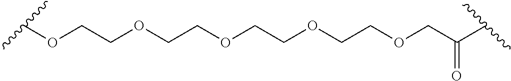 [SMILES: *OCCOCCOCCOCCOCC(*)=O]

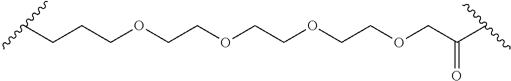 [SMILES: *CCCOCCOCCOCCOCC(*)=O]

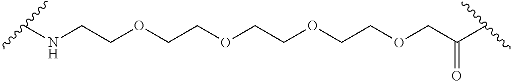 [SMILES: *NCCOCCOCCOCCOCC(*)=O]

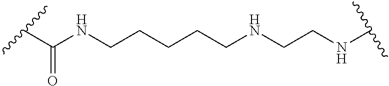 [SMILES: *NCCNCCCCCNC(*)=O]

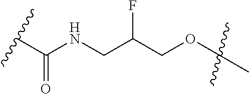 [SMILES: *OCC(F)CNC(*)=O]

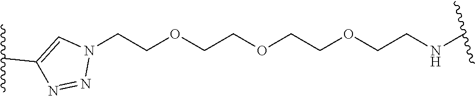 [SMILES: *NCCOCCOCCOCCn1cc(*)nn1]

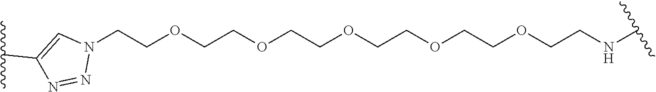 [SMILES: *NCCOCCOCCOCCOCCOCCn1cc(*)nn1]

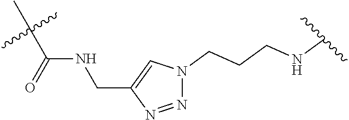 [SMILES: *NCCCn1cc(CNC(*)=O)nn1]

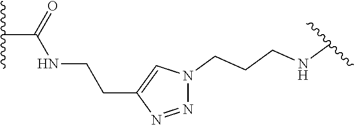 [SMILES: *NCCCn1cc(CCNC(*)=O)nn1]

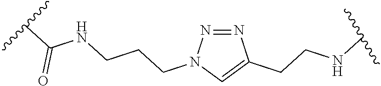 [SMILES: *NCCc1cn(CCCNC(*)=O)nn1]

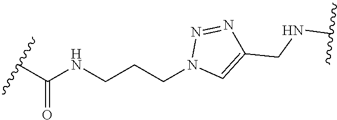 [SMILES: *NCc1cn(CCCNC(*)=O)nn1]

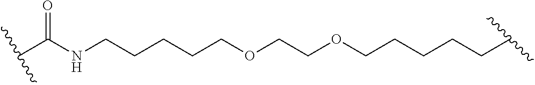 [SMILES: *CCCCCOCCOCCCCCNC(*)=O]

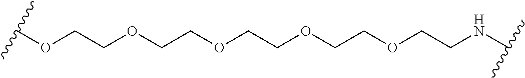 [SMILES: *NCCOCCOCCOCCOCCO*]

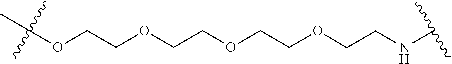 [SMILES: *NCCOCCOCCOCCO*]

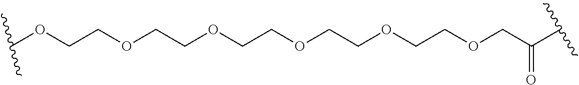 [SMILES: *OCCOCCOCCOCCOCCOCC(*)=O]

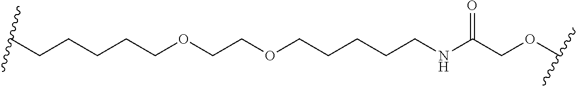 [SMILES: *CCCCCOCCOCCCCCNC(=O)CO*]

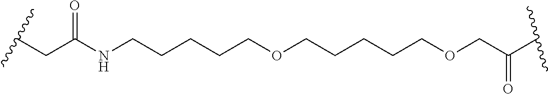 [SMILES: *CC(=O)NCCCCCOCCCCCOCC(*)=O]

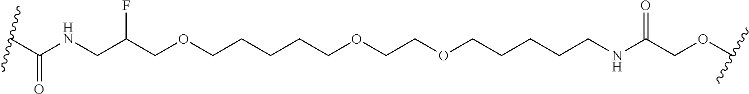 [SMILES: *OCC(=O)NCCCCCOCCOCCCCCOCC(F)CNC(*)=O]

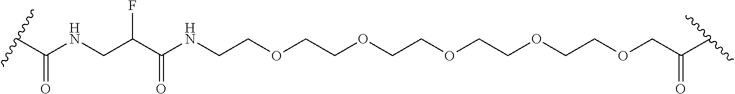 [SMILES: *C(=O)COCCOCCOCCOCCOCCNC(=O)C(F)CNC(*)=O]

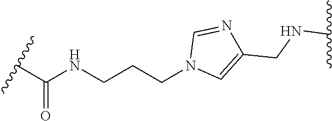 [SMILES: *NCc1cn(CCCNC(*)=O)cn1]

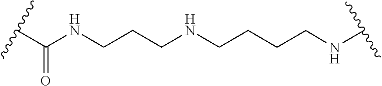 [SMILES: *NCCCCNCCCNC(*)=O]

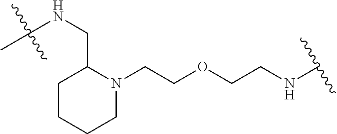 [SMILES: *NCCOCCN1CCCCC1CN*]

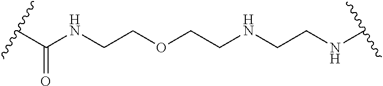 [SMILES: *NCCNCCOCCNC(*)=O]

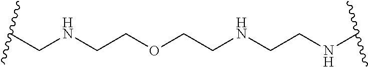 [SMILES: *CNCCOCCNCCN*]

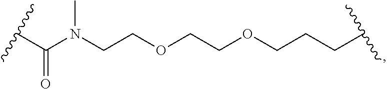 [SMILES: *CCCOCCOCCN(C)C(*)=O]

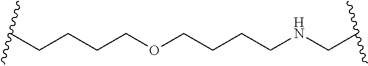 [SMILES: *CCCCOCCCCNC*]

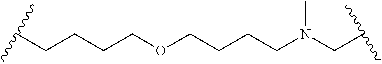 [SMILES: *CCCCOCCCCN(C)C*]

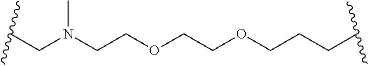 [SMILES: *CCCOCCOCCN(C)C*]

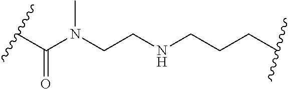 [SMILES: *CCCNCCN(C)C(*)=O]

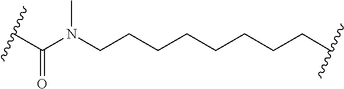 [SMILES: *CCCCCCCCN(C)C(*)=O]

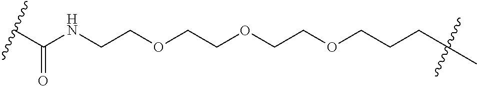 [SMILES: *CCCOCCOCCOCCNC(*)=O]

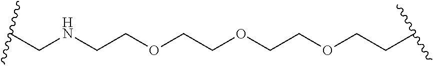 [SMILES: *CCOCCOCCOCCNC*]

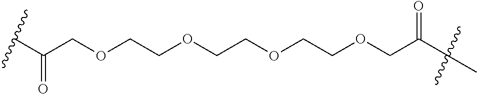 [SMILES: *C(=O)COCCOCCOCCOCC(*)=O]

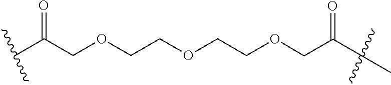 [SMILES: *C(=O)COCCOCCOCC(*)=O]

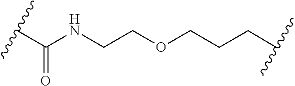 [SMILES: *CCCOCCNC(*)=O]

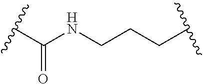 [SMILES: *CCCNC(*)=O]

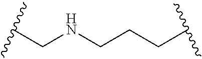 [SMILES: *CCCNC*]

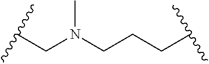 [SMILES: *CCCN(C)C*]

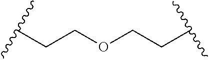 [SMILES: *CCOCC*]

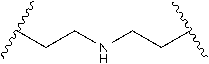 [SMILES: *CCNCC*]

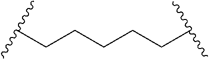 [SMILES: *CCCCC*]

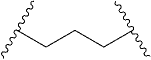 [SMILES: *CCC*]

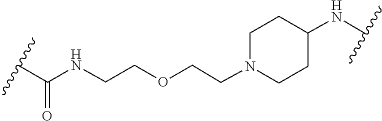 [SMILES: *NC1CCN(CCOCCNC(*)=O)CC1]

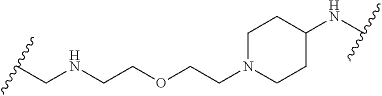 [SMILES: *CNCCOCCN1CCC(N*)CC1]

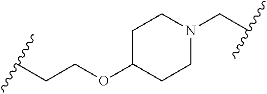 [SMILES: *CCOC1CCN(C*)CC1]

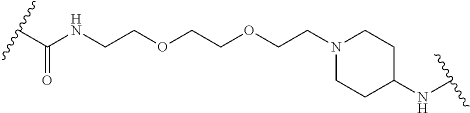 [SMILES: *NC1CCN(CCOCCOCCNC(*)=O)CC1]

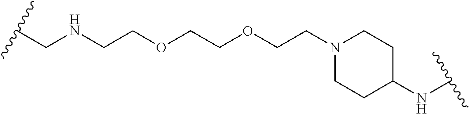 [SMILES: *CNCCOCCOCCN1CCC(N*)CC1]

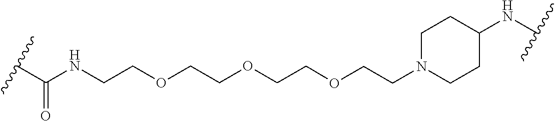 [SMILES: *NC1CCN(CCOCCOCCOCCNC(*)=O)CC1]

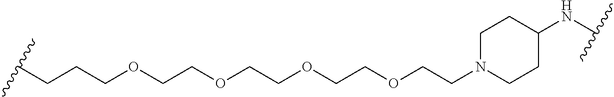 [SMILES: *CCCOCCOCCOCCOCCN1CCC(N*)CC1]

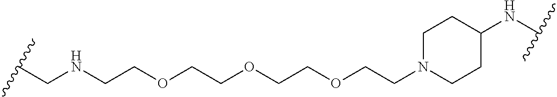 [SMILES: *CNCCOCCOCCOCCN1CCC(N*)CC1]

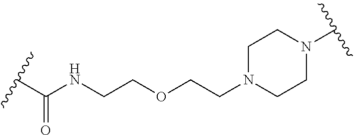 [SMILES: *C(=O)NCCOCCN1CCN(*)CC1]

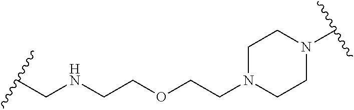 [SMILES: *CNCCOCCN1CCN(*)CC1]

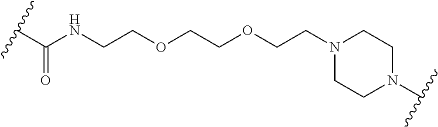 [SMILES: *C(=O)NCCOCCOCCN1CCN(*)CC1]

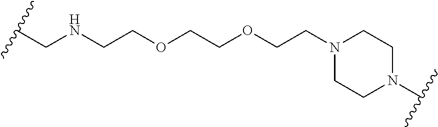 [SMILES: *CNCCOCCOCCN1CCN(*)CC1]

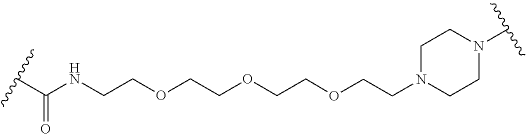 [SMILES: *C(=O)NCCOCCOCCOCCN1CCN(*)CC1]

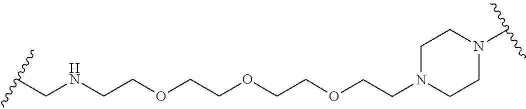 [SMILES: *CNCCOCCOCCOCCN1CCN(*)CC1]

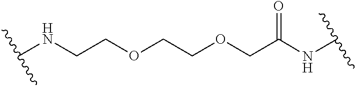 [SMILES: *NCCOCCOCC(=O)N*]

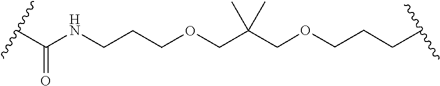 [SMILES: *CCCOCC(C)(C)COCCCNC(*)=O]

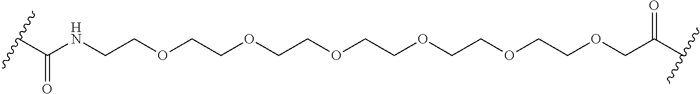 [SMILES: *C(=O)COCCOCCOCCOCCOCCOCCNC(*)=O]

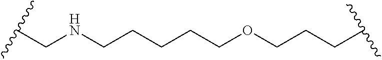 [SMILES: *CCCOCCCCCNC*]

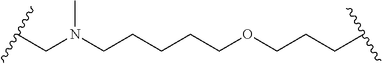 [SMILES: *CCCOCCCCCN(C)C*]

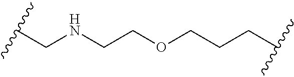 [SMILES: *CCCOCCNC*]

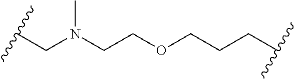 [SMILES: *CCCOCCN(C)C*]

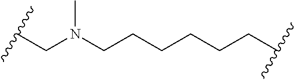 [SMILES: *CCCCCCN(C)C*]

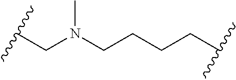 [SMILES: *CCCCN(C)C*]

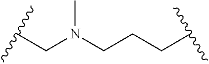 [SMILES: *CCCN(C)C*]

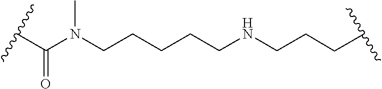 [SMILES: *CCCNCCCCCN(C)C(*)=O]

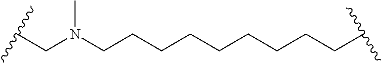 [SMILES: *CCCCCCCCCN(C)C*]

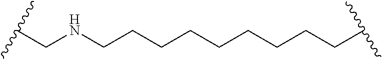 [SMILES: *CCCCCCCCCNC*]

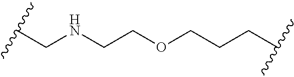 [SMILES: *CCCOCCNC*]

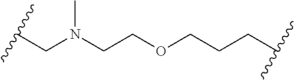 [SMILES: *CCCOCCN(C)C*]

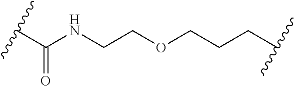 [SMILES: *CCCOCCNC(*)=O]

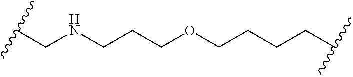 [SMILES: *CCCCOCCCNC*]

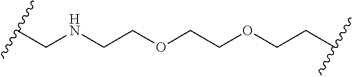 [SMILES: *CCOCCOCCNC*]

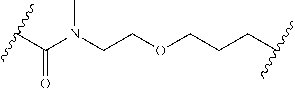 [SMILES: *CCCOCCN(C)C(*)=O]

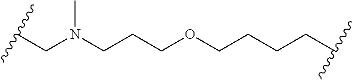 [SMILES: *CCCCOCCCN(C)C*]

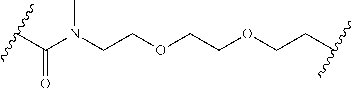 [SMILES: *CCOCCOCCN(C)C(*)=O]

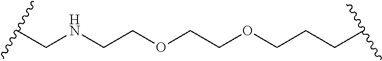 [SMILES: *CCCOCCOCCNC*]

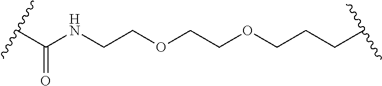 [SMILES: *CCCOCCOCCNC(*)=O]

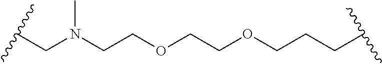 [SMILES: *CCCOCCOCCN(C)C*]

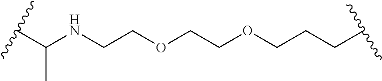 [SMILES: *CCCOCCOCCNC(*)C]

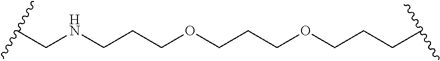 [SMILES: *CCCOCCCOCCCNC*]

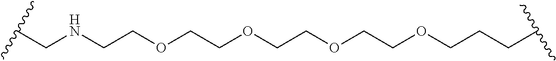 [SMILES: *CCCOCCOCCOCCOCCNC*]

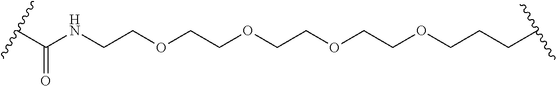 [SMILES: *CCCOCCOCCOCCOCCNC(*)=O]

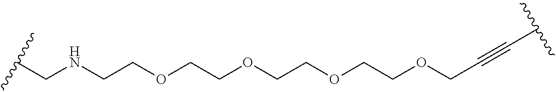 [SMILES: *C#CCOCCOCCOCCOCCNC*]

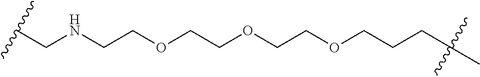 [SMILES: *CCCOCCOCCOCCNC*]

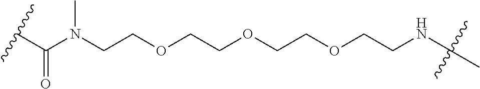 [SMILES: *NCCOCCOCCOCCN(C)C(*)=O]

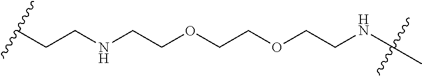 [SMILES: *CCNCCOCCOCCN*]

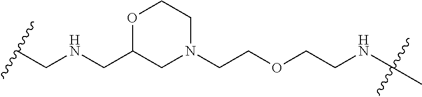 [SMILES: *CNCC1CN(CCOCCN*)CCO1]

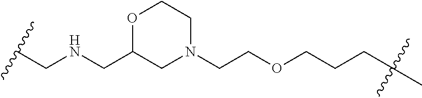 [SMILES: *CCCOCCN1CCOC(CNC*)C1]

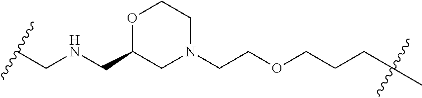 [SMILES: *CCCOCCN1CCO[C@H](CNC*)C1]

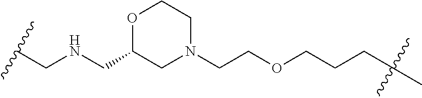 [SMILES: *CCCOCCN1CCO[C@@H](CNC*)C1]

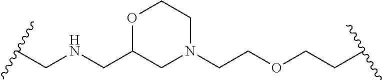 [SMILES: *CCOCCN1CCOC(CNC*)C1]

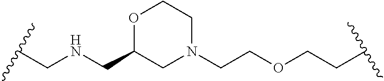 [SMILES: *CCOCCN1CCO[C@H](CNC*)C1]

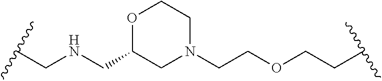 [SMILES: *CCOCCN1CCO[C@@H](CNC*)C1]

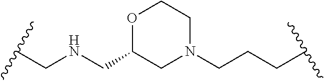 [SMILES: *CCCN1CCO[C@@H](CNC*)C1]

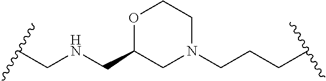 [SMILES: *CCCN1CCO[C@H](CNC*)C1]

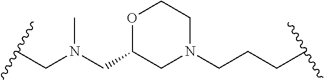 [SMILES: *CCCN1CCO[C@@H](CN(C)C*)C1]

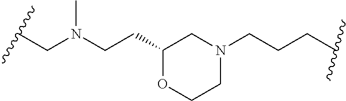 [SMILES: *CCCN1CCO[C@H](CCN(C)C*)C1]

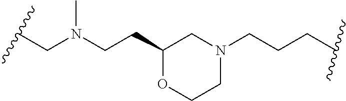 [SMILES: *CCCN1CCO[C@@H](CCN(C)C*)C1]

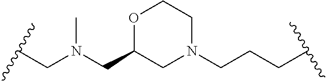 [SMILES: *CCCN1CCO[C@H](CN(C)C*)C1]

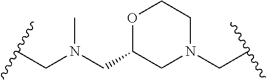 [SMILES: *CN(C)C[C@H]1CN(C*)CCO1]

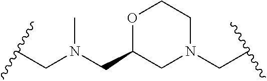 [SMILES: *CN(C)C[C@@H]1CN(C*)CCO1]

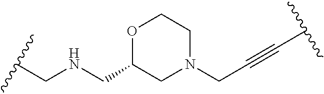 [SMILES: *C#CCN1CCO[C@@H](CNC*)C1]

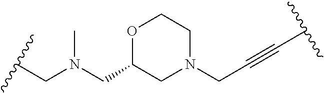 [SMILES: *C#CCN1CCO[C@@H](CN(C)C*)C1]

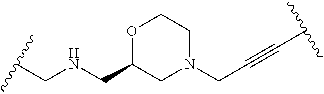 [SMILES: *C#CCN1CCO[C@H](CNC*)C1]

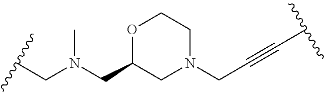 [SMILES: *C#CCN1CCO[C@H](CN(C)C*)C1]

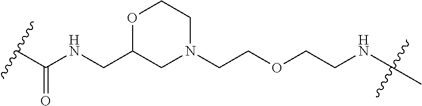 [SMILES: *NCCOCCN1CCOC(CNC(*)=O)C1]

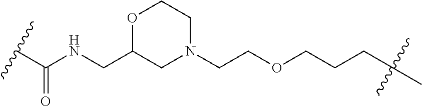 [SMILES: *CCCOCCN1CCOC(CNC(*)=O)C1]

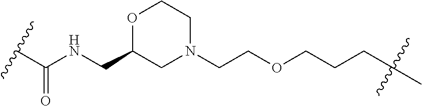 [SMILES: *CCCOCCN1CCO[C@H](CNC(*)=O)C1]

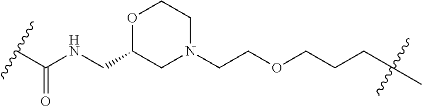 [SMILES: *CCCOCCN1CCO[C@@H](CNC(*)=O)C1]

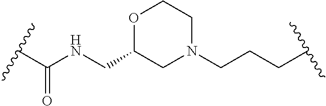 [SMILES: *CCCN1CCO[C@@H](CNC(*)=O)C1]

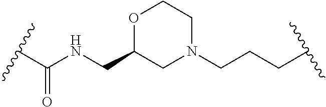 [SMILES: *CCCN1CCO[C@H](CNC(*)=O)C1]

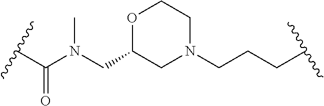 [SMILES: *CCCN1CCO[C@@H](CN(C)C(*)=O)C1]

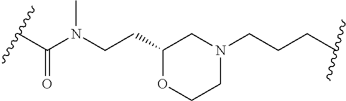 [SMILES: *CCCN1CCO[C@H](CCN(C)C(*)=O)C1]

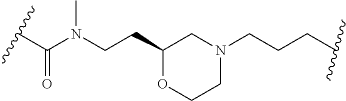 [SMILES: *CCCN1CCO[C@@H](CCN(C)C(*)=O)C1]

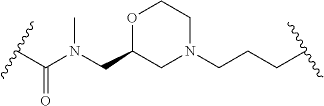 [SMILES: *CCCN1CCO[C@H](CN(C)C(*)=O)C1]

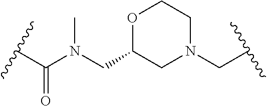 [SMILES: *CN1CCO[C@@H](CN(C)C(*)=O)C1]

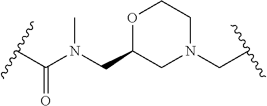 [SMILES: *CN1CCO[C@H](CN(C)C(*)=O)C1]

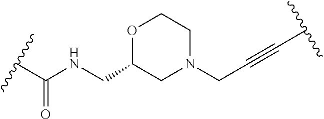 [SMILES: *C#CCN1CCO[C@@H](CNC(*)=O)C1]

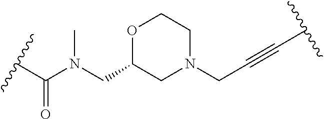 [SMILES: *C#CCN1CCO[C@@H](CN(C)C(*)=O)C1]

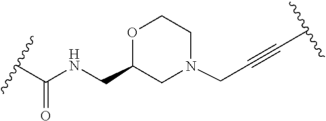 [SMILES: *C#CCN1CCO[C@H](CNC(*)=O)C1]

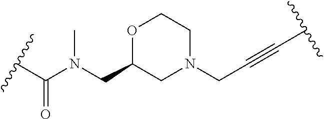 [SMILES: *C#CCN1CCO[C@H](CN(C)C(*)=O)C1]

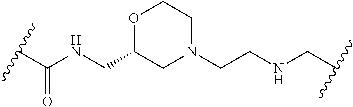 [SMILES: *CNCCN1CCO[C@@H](CNC(*)=O)C1]

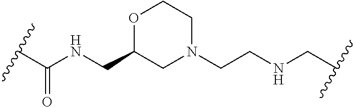 [SMILES: *CNCCN1CCO[C@H](CNC(*)=O)C1]

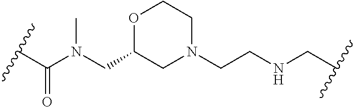 [SMILES: *CNCCN1CCO[C@@H](CN(C)C(*)=O)C1]

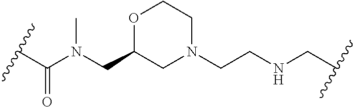 [SMILES: *CNCCN1CCO[C@H](CN(C)C(*)=O)C1]

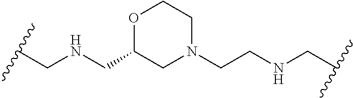 [SMILES: *CNCCN1CCO[C@@H](CNC*)C1]

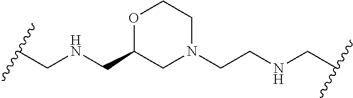 [SMILES: *CNCCN1CCO[C@H](CNC*)C1]

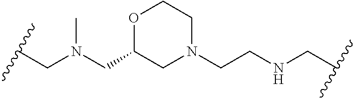 [SMILES: *CNCCN1CCO[C@@H](CN(C)C*)C1]

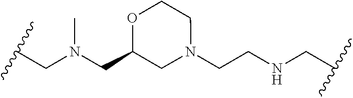 [SMILES: *CNCCN1CCO[C@H](CN(C)C*)C1]

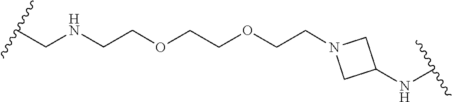 [SMILES: *CNCCOCCOCCN1CC(N*)C1]

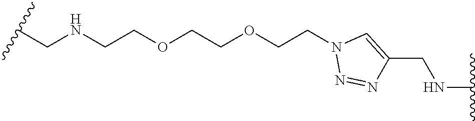 [SMILES: *CNCCOCCOCCn1cc(CN*)nn1]

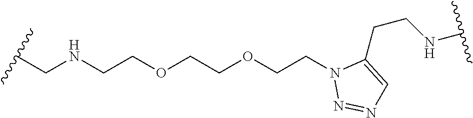 [SMILES: *CNCCOCCOCCn1nncc1CCN*]

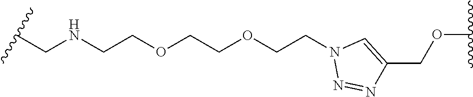 [SMILES: *CNCCOCCOCCn1cc(CO*)nn1]

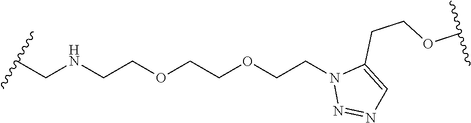 [SMILES: *CNCCOCCOCCn1nncc1CCO*]

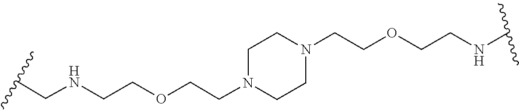 [SMILES: *CNCCOCCN1CCN(CCOCCN*)CC1]

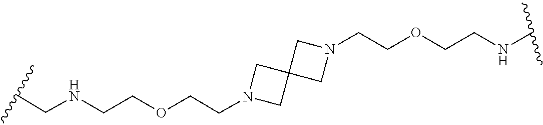 [SMILES: *CNCCOCCN1CC2(CN(CCOCCN*)C2)C1]

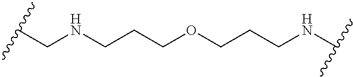 [SMILES: *CNCCCOCCCN*]

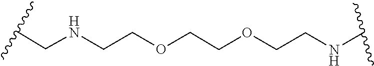 [SMILES: *CNCCOCCOCCN*]

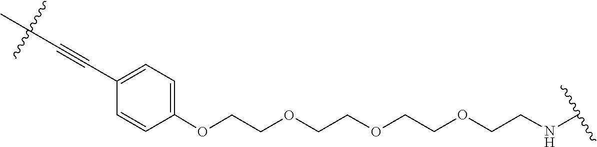 [SMILES: *C#Cc1ccc(OCCOCCOCCOCCN*)cc1]

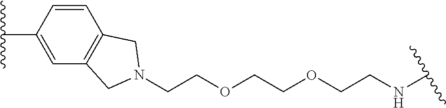 [SMILES: *NCCOCCOCCN1Cc2ccc(*)cc2C1]

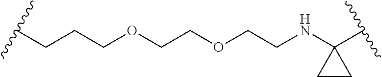 [SMILES: *CCCOCCOCCNC1(*)CC1]

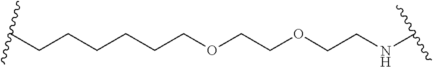 [SMILES: *CCCCCCOCCOCCN*]

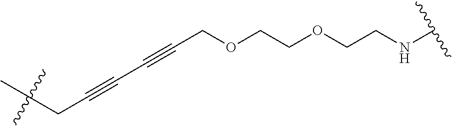 [SMILES: *CC#CC#CCOCCOCCN*]

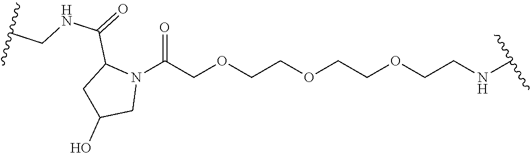 [SMILES: *CNC(=O)C1CC(O)CN1C(=O)COCCOCCOCCN*]

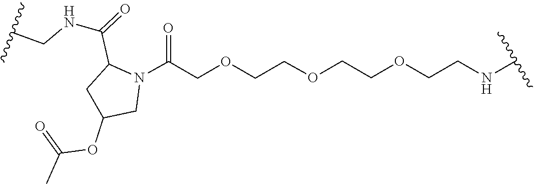 [SMILES: *CNC(=O)C1CC(OC(C)=O)CN1C(=O)COCCOCCOCCN*]

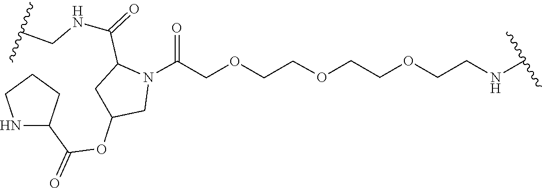 [SMILES: *CNC(=O)C1CC(OC(=O)C2CCCN2)CN1C(=O)COCCOCCOCCN*]

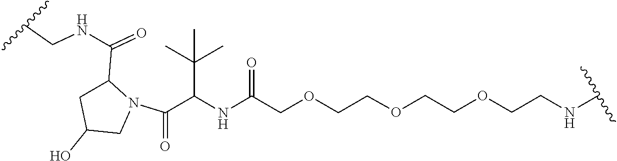 [SMILES: *CNC(=O)C1CC(O)CN1C(=O)C(NC(=O)COCCOCCOCCN*)C(C)(C)C]

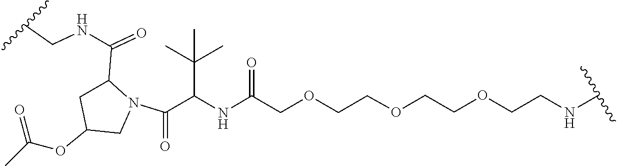 [SMILES: *CNC(=O)C1CC(OC(C)=O)CN1C(=O)C(NC(=O)COCCOCCOCCN*)C(C)(C)C]

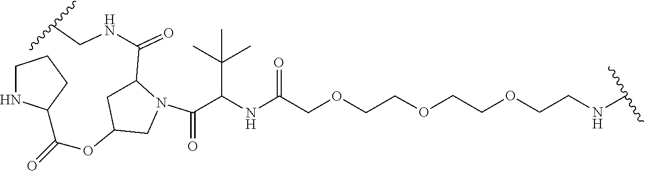 [SMILES: *CNC(=O)C1CC(OC(=O)C2CCCN2)CN1C(=O)C(NC(=O)COCCOCCOCCN*)C(C)(C)C]

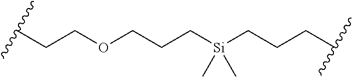 [SMILES: *CCC[Si](C)(C)CCCOCC*]

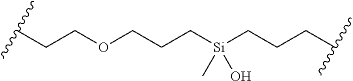 [SMILES: *CCC[Si](C)(O)CCCOCC*]

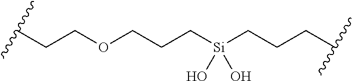 [SMILES: *CCC[Si](O)(O)CCCOCC*]

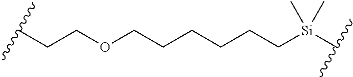 [SMILES: *CCOCCCCCC[Si](*)(C)C]

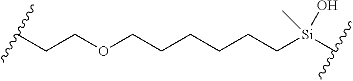 [SMILES: *CCOCCCCCC[Si](*)(C)O]

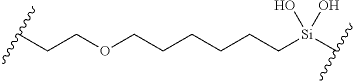 [SMILES: *CCOCCCCCC[Si](*)(O)O]

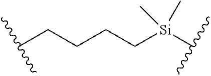 [SMILES: *CCCC[Si](*)(C)C]

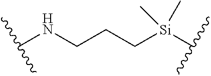 [SMILES: *NCCC[Si](*)(C)C]

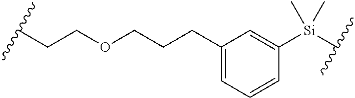 [SMILES: *CCOCCCc1cccc([Si](*)(C)C)c1]

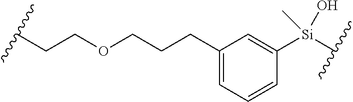 [SMILES: *CCOCCCc1cccc([Si](*)(C)O)c1]

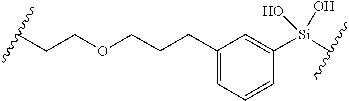 [SMILES: *CCOCCCc1cccc([Si](*)(O)O)c1]

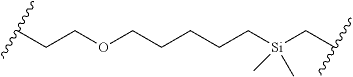 [SMILES: *CCOCCCCC[Si](C)(C)C*]

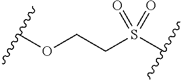 [SMILES: *OCCS(*)(=O)=O]

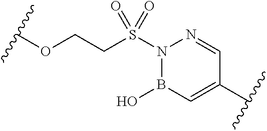 [SMILES: *OCCS(=O)(=O)N1N=CC(*)=CB1O]

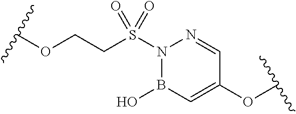 [SMILES: *OCCS(=O)(=O)N1N=CC(O*)=CB1O]

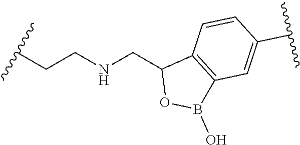 [SMILES: *CCNCC1OB(O)c2cc(*)ccc21]

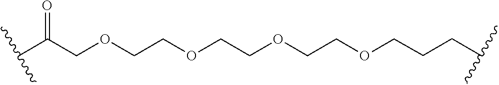 [SMILES: *CCCOCCOCCOCCOCC(*)=O]

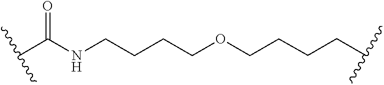 [SMILES: *CCCCOCCCCNC(*)=O]

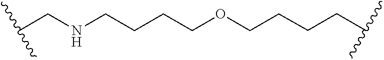 [SMILES: *CCCCOCCCCNC*]

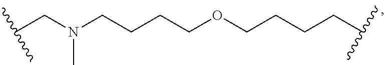 [SMILES: *CCCCOCCCCN(C)C*]

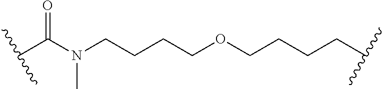 [SMILES: *CCCCOCCCCN(C)C(*)=O]

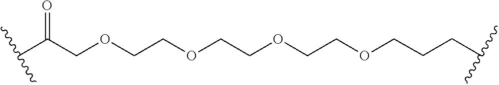 [SMILES: *CCCOCCOCCOCCOCC(*)=O]

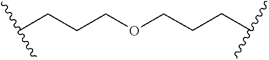 [SMILES: *CCCOCCC*]

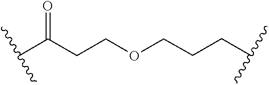 [SMILES: *CCCOCCC(*)=O]

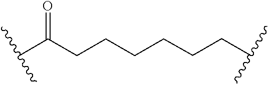 [SMILES: *CCCCCCC(*)=O]

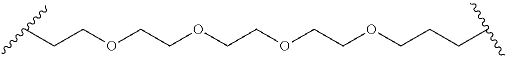 [SMILES: *CCCOCCOCCOCCOCC*]

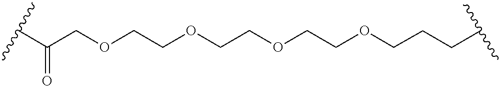 [SMILES: *CCCOCCOCCOCCOCC(*)=O]

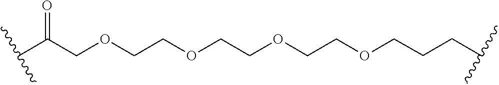 [SMILES: *CCCOCCOCCOCCOCC(*)=O]

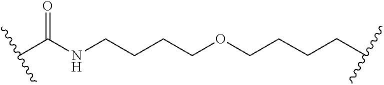 [SMILES: *CCCCOCCCCNC(*)=O]

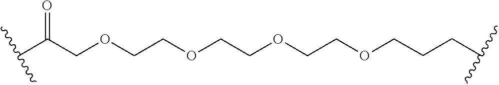 [SMILES: *CCCOCCOCCOCCOCC(*)=O]

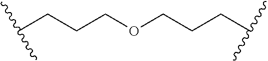 [SMILES: *CCCOCCC*]

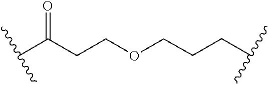 [SMILES: *CCCOCCC(*)=O]

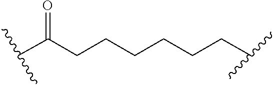 [SMILES: *CCCCCCC(*)=O]

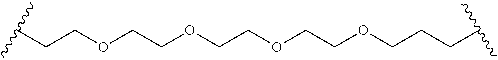 [SMILES: *CCCOCCOCCOCCOCC*]

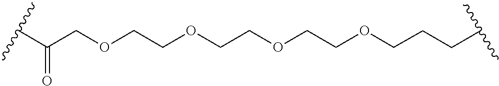 [SMILES: *CCCOCCOCCOCCOCC(*)=O]

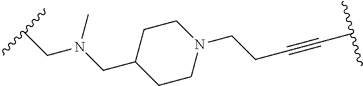 [SMILES: *C#CCCN1CCC(CN(C)C*)CC1]

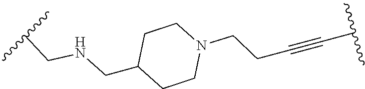 [SMILES: *C#CCCN1CCC(CNC*)CC1]

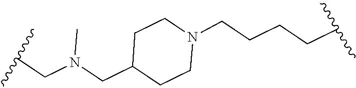 [SMILES: *CCCCN1CCC(CN(C)C*)CC1]

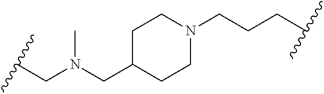 [SMILES: *CCCN1CCC(CN(C)C*)CC1]

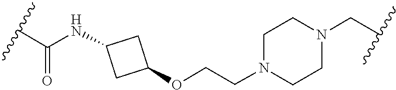 [SMILES: *CN1CCN(CCO[C@H]2C[C@H](NC(*)=O)C2)CC1]

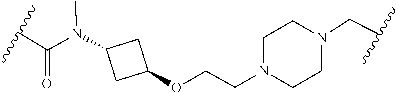 [SMILES: *CN1CCN(CCO[C@H]2C[C@H](N(C)C(*)=O)C2)CC1]

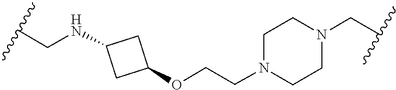 [SMILES: *CN[C@H]1C[C@H](OCCN2CCN(C*)CC2)C1]

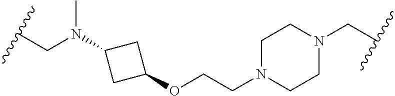 [SMILES: *CN1CCN(CCO[C@H]2C[C@H](N(C)C*)C2)CC1]

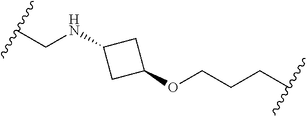 [SMILES: *CCCO[C@H]1C[C@H](NC*)C1]

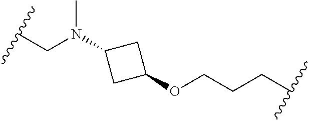 [SMILES: *CCCO[C@H]1C[C@H](N(C)C*)C1]

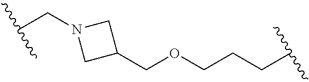 [SMILES: *CCCOCC1CN(C*)C1]

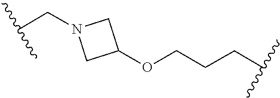 [SMILES: *CCCOC1CN(C*)C1]

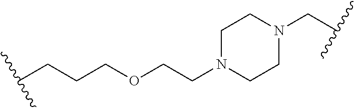 [SMILES: *CCCOCCN1CCN(C*)CC1]

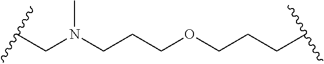 [SMILES: *CCCOCCCN(C)C*]

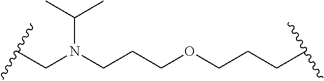 [SMILES: *CCCOCCCN(C*)C(C)C]

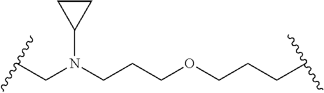 [SMILES: *CCCOCCCN(C*)C1CC1]

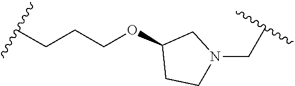 [SMILES: *CCCO[C@@H]1CCN(C*)C1]

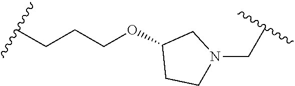 [SMILES: *CCCO[C@H]1CCN(C*)C1]

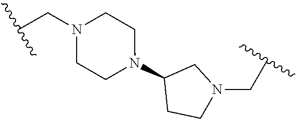 [SMILES: *CN1CCN([C@@H]2CCN(C*)C2)CC1]

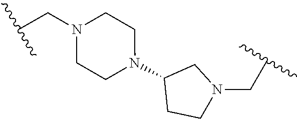 [SMILES: *CN1CCN([C@H]2CCN(C*)C2)CC1]

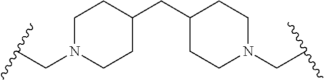 [SMILES: *CN1CCC(CC2CCN(C*)CC2)CC1]

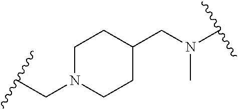 [SMILES: *CN1CCC(CN(*)C)CC1]

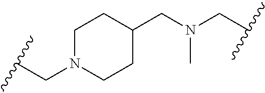 [SMILES: *CN(C)CC1CCN(C*)CC1]

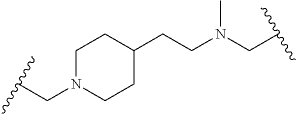 [SMILES: *CN(C)CCC1CCN(C*)CC1]

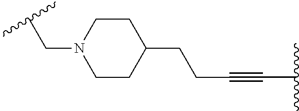 [SMILES: *C#CCCC1CCN(C*)CC1]

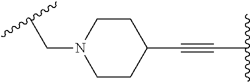 [SMILES: *C#CC1CCN(C*)CC1]

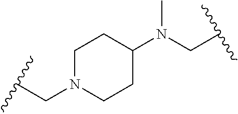 [SMILES: *CN1CCC(N(C)C*)CC1]

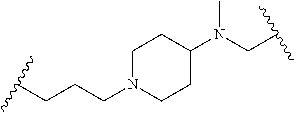 [SMILES: *CCCN1CCC(N(C)C*)CC1]

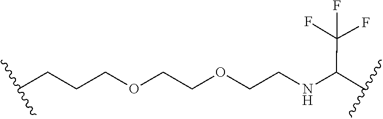 [SMILES: *CCCOCCOCCNC(*)C(F)(F)F]

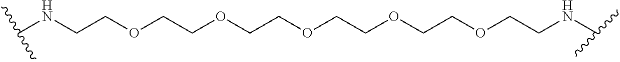 [SMILES: *NCCOCCOCCOCCOCCOCCN*]

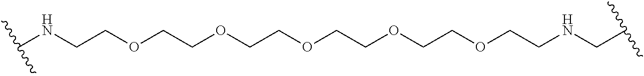 [SMILES: *CNCCOCCOCCOCCOCCOCCN*]

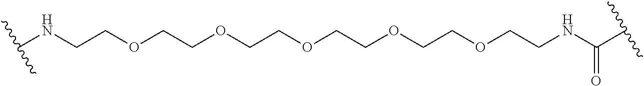 [SMILES: *NCCOCCOCCOCCOCCOCCNC(*)=O]

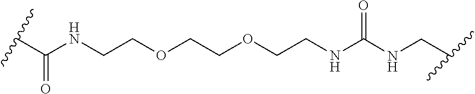 [SMILES: *CNC(=O)NCCOCCOCCNC(*)=O]

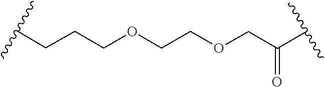 [SMILES: *CCCOCCOCC(*)=O]

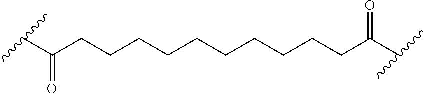 [SMILES: *C(=O)CCCCCCCCCCC(*)=O]

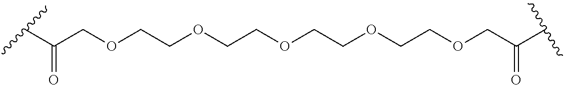 [SMILES: *C(=O)COCCOCCOCCOCCOCC(*)=O]

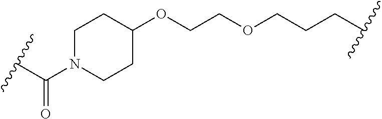 [SMILES: *CCCOCCOC1CCN(C(*)=O)CC1]

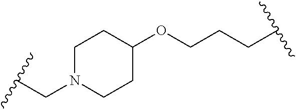 [SMILES: *CCCOC1CCN(C*)CC1]

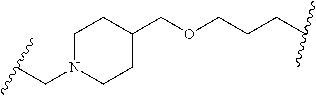 [SMILES: *CCCOCC1CCN(C*)CC1]

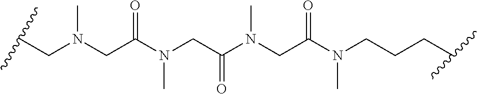 [SMILES: *CCCN(C)C(=O)CN(C)C(=O)CN(C)C(=O)CN(C)C*]

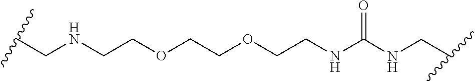 [SMILES: *CNCCOCCOCCNC(=O)NC*]

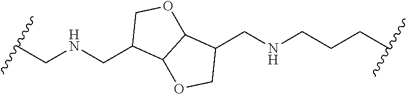 [SMILES: *CCCNCC1COC2C(CNC*)COC12]

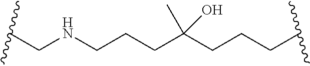 [SMILES: *CCCC(C)(O)CCCNC*]

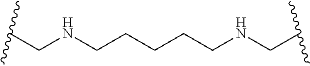 [SMILES: *CNCCCCCNC*]

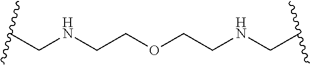 [SMILES: *CNCCOCCNC*]

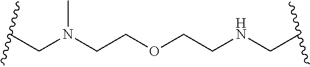 [SMILES: *CNCCOCCN(C)C*]

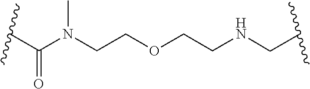 [SMILES: *CNCCOCCN(C)C(*)=O]

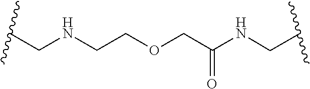 [SMILES: *CNCCOCC(=O)NC*]

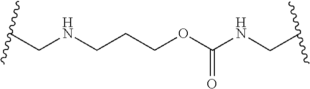 [SMILES: *CNCCCOC(=O)NC*]

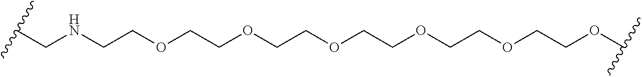 [SMILES: *CNCCOCCOCCOCCOCCOCCO*]

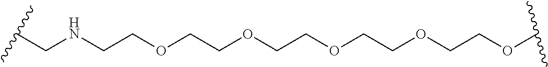 [SMILES: *CNCCOCCOCCOCCOCCO*]

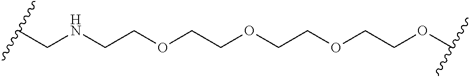 [SMILES: *CNCCOCCOCCOCCO*]

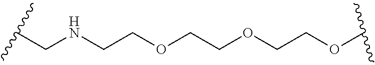 [SMILES: *CNCCOCCOCCO*]

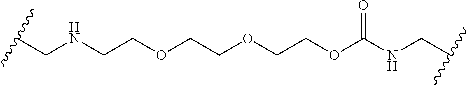 [SMILES: *CNCCOCCOCCOC(=O)NC*]

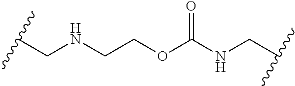 [SMILES: *CNCCOC(=O)NC*]

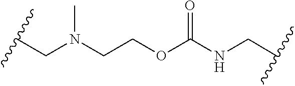 [SMILES: *CNC(=O)OCCN(C)C*]

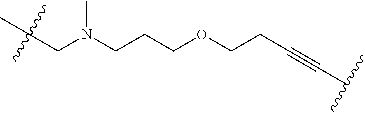 [SMILES: *C#CCCOCCCN(C)C*]

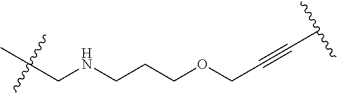 [SMILES: *C#CCOCCCNC*]

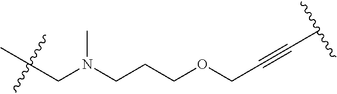 [SMILES: *C#CCOCCCN(C)C*]

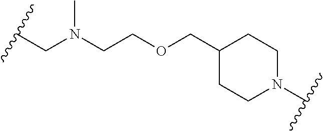 [SMILES: *CN(C)CCOCC1CCN(*)CC1]

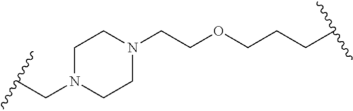 [SMILES: *CCCOCCN1CCN(C*)CC1]

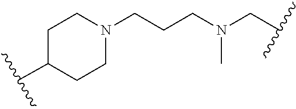 [SMILES: *CN(C)CCCN1CCC(*)CC1]

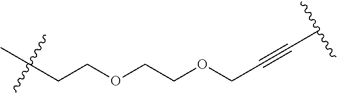 [SMILES: *C#CCOCCOCC*]

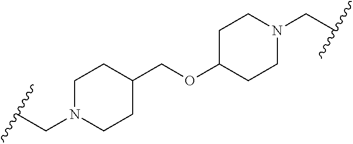 [SMILES: *CN1CCC(COC2CCN(C*)CC2)CC1]

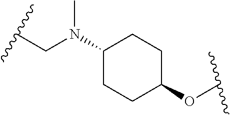 [SMILES: *CN(C)[C@H]1CC[C@H](O*)CC1]

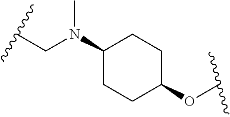 [SMILES: *CN(C)[C@H]1CC[C@@H](O*)CC1]

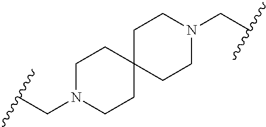 [SMILES: *CN1CCC2(CC1)CCN(C*)CC2]

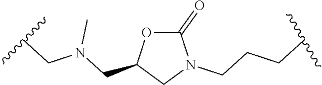 [SMILES: *CCCN1C[C@@H](CN(C)C*)OC1=O]

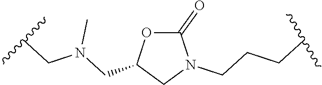 [SMILES: *CCCN1C[C@H](CN(C)C*)OC1=O]

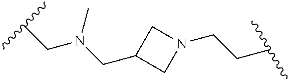 [SMILES: *CCN1CC(CN(C)C*)C1]

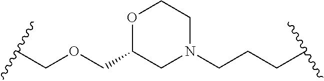 [SMILES: *CCCN1CCO[C@@H](COC*)C1]

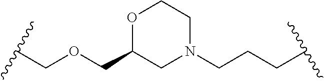 [SMILES: *CCCN1CCO[C@H](COC*)C1]

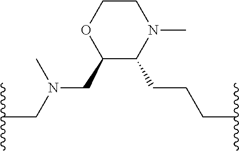 [SMILES: *CCC[C@@H]1[C@@H](CN(C)C*)OCCN1C]

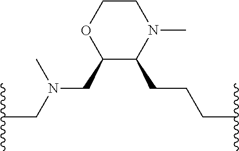 [SMILES: *CCC[C@H]1[C@@H](CN(C)C*)OCCN1C]

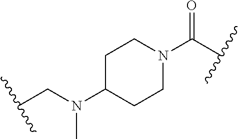 [SMILES: *CN(C)C1CCN(C(*)=O)CC1]

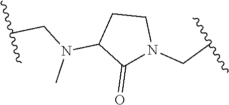 [SMILES: *CN1CCC(N(C)C*)C1=O]

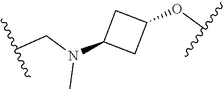 [SMILES: *CN(C)[C@H]1C[C@H](O*)C1]

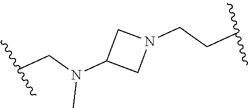 [SMILES: *CCN1CC(N(C)C*)C1]

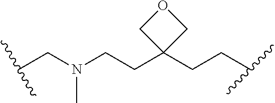 [SMILES: *CCC1(CCN(C)C*)COC1]

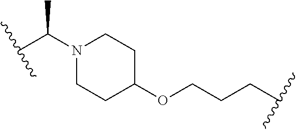 [SMILES: *CCCOC1CCN([C@@H](*)C)CC1]

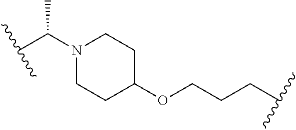 [SMILES: *CCCOC1CCN([C@H](*)C)CC1]

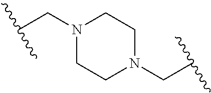 [SMILES: *CN1CCN(C*)CC1]

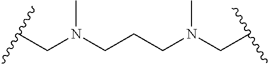 [SMILES: *CN(C)CCCN(C)C*]

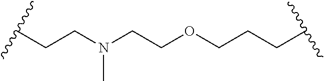 [SMILES: *CCCOCCN(C)CC*]

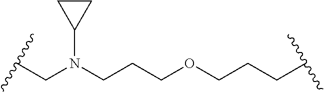 [SMILES: *CCCOCCCN(C*)C1CC1]

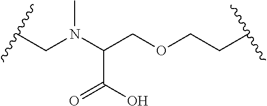 [SMILES: *CCOCC(C(=O)O)N(C)C*]

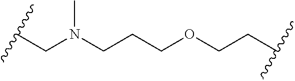 [SMILES: *CCOCCCN(C)C*]

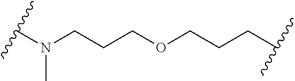 [SMILES: *CCCOCCCN(*)C]

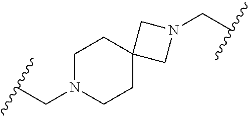 [SMILES: *CN1CCC2(CC1)CN(C*)C2]

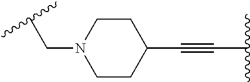 [SMILES: *C#CC1CCN(C*)CC1]

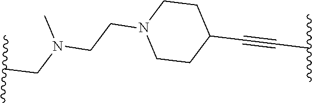 [SMILES: *C#CC1CCN(CCN(C)C*)CC1]

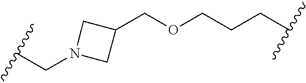 [SMILES: *CCCOCC1CN(C*)C1]

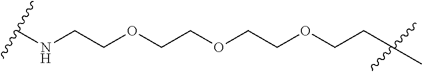 [SMILES: *CCOCCOCCOCCN*]

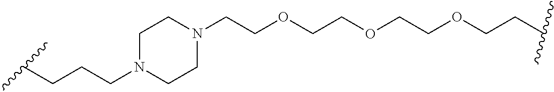 [SMILES: *CCCN1CCN(CCOCCOCCOCC*)CC1]

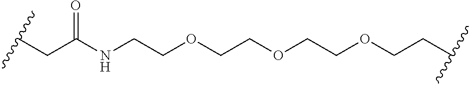 [SMILES: *CCOCCOCCOCCNC(=O)C*]

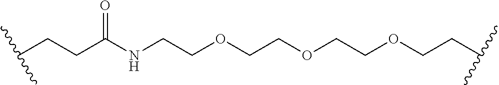 [SMILES: *CCOCCOCCOCCNC(=O)CC*]

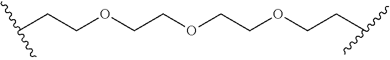 [SMILES: *CCOCCOCCOCC*]

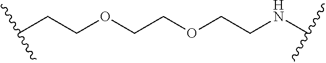 [SMILES: *CCOCCOCCN*]

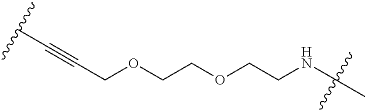 [SMILES: *C#CCOCCOCCN*]

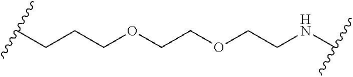 [SMILES: *CCCOCCOCCN*]

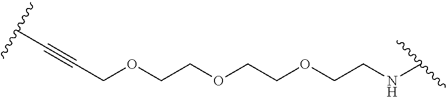 [SMILES: *C#CCOCCOCCOCCN*]

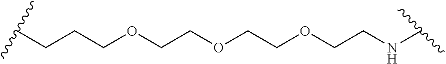 [SMILES: *CCCOCCOCCOCCN*]

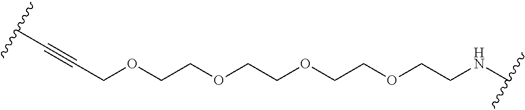 [SMILES: *C#CCOCCOCCOCCOCCN*]

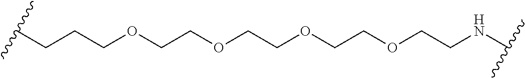 [SMILES: *CCCOCCOCCOCCOCCN*]

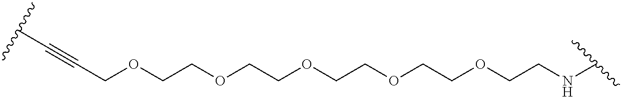 [SMILES: *C#CCOCCOCCOCCOCCOCCN*]

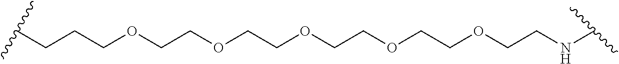 [SMILES: *CCCOCCOCCOCCOCCOCCN*]

In some embodiments, L¹ is selected from those depicted in Table 1, as shown above.

In some embodiments, L¹ is selected from those depicted in Table 1A, as shown above. In some embodiments, L¹ is selected from those depicted in Table 1B, as shown above. In some embodiments, L¹ is selected from those depicted in Table 1C, as shown above.

In some embodiments, compounds of Formula A include but are not limited to

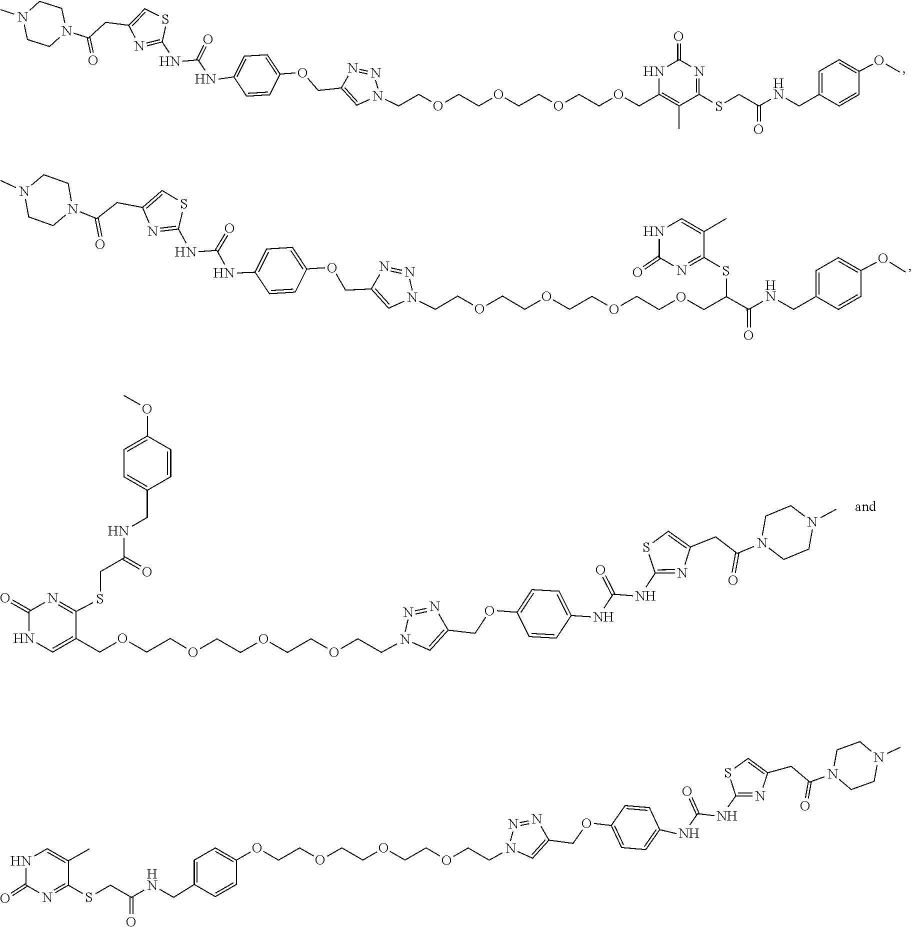

### Definitions

As used herein, a “nucleoside” refers to a molecule consisting of a guanine (G), adenine (A), thymine (T), uridine (U), or cytidine (C) base covalently linked to a pentose sugar, whereas “nucleotide” or “mononucleotide” refers to a nucleoside phosphorylated at one of the hydroxyl groups of the pentose sugar. “Nucleoside” also encompasses analogs of G, A, T, C, or U and natural or non-natural nucleic acid components wherein the base, sugar, and/or phosphate backbone have been modified or replaced. Nucleoside analogs are known in the art and include those described herein. Also included are endogenous, post-transcriptionally modified nucleosides, such as methylated nucleosides.

Linear nucleic acid molecules are said to have a “5′ terminus” (5′-end) and a “3′ terminus” (3′-end) because, except with respect to adenylation (as described elsewhere herein), mononucleotides are joined in one direction via a phosphodiester linkage (or analog thereof) to make oligonucleotides, in a manner such that a phosphate (or analog thereof) on the 5′ carbon of one mononucleotide sugar is joined to an oxygen on the 3′ carbon of the sugar of its neighboring mononucleotide. Therefore, an end of an oligonucleotide is referred to as the “5′ end” if its 5′ phosphate (or analog thereof) is not linked to the oxygen of the 3′ carbon of a mononucleotide sugar, and as the “3′ end” if its 3′ oxygen is not linked to a 5′ phosphate (or analog thereof) of a subsequent mononucleotide sugar. A “terminal nucleotide,” as used herein, is the nucleotide at the end position of the 3′ or 5′ terminus. The 3′ or 5′ terminus may alternatively end in a 3′-OH or 5′-OH if the terminal nucleotide is not phosphorylated.

As used herein, the term “nucleic acid” refers to a covalently linked sequence of nucleotides in which the 3′ position of the sugar of one nucleotide is joined by a phosphodiester bond to the 5′ position of the sugar of the next nucleotide (i.e., a 3′ to 5′ phosphodiester bond), and in which the nucleotides are linked in specific sequence; i.e., a linear order of nucleotides. “Nucleic acid” includes analogs of the foregoing wherein one or more nucleotides are modified at the base, sugar, or phosphodiester. Such analogs are known in the art and include those described elsewhere herein. As used herein, “polynucleotide” or “polynucleic acid” refers to a long nucleic acid sequence (or analog thereof) of many nucleotides. For example, but without limitation, a polynucleotide (or polynucleic acid) may be greater than 60, 61-1,000, or 201-1,000, or greater than 1,000 nucleotides in length. As used herein, an “oligonucleotide” or “oligonucleic acid” is a short polynucleotide or a portion of a polynucleotide. For example, but without limitation, an oligonucleotide may be between 5-10, 10-60, or 10-200 nucleotides in length.

In some embodiments, a nucleic acid, oligonucleotide, or polynucleotide consists of, consists primarily of, or is mostly 2′-deoxyribonucleotides (DNA) or ribonucleotides (RNA). In some embodiments, an oligonucleotide consists of or comprises 2′-deoxyribonucleotides (DNA). In some embodiments, the oligonucleotide consists of or comprises ribonucleotides (RNA). In some embodiments, the oligonucleotide is a DNA-RNA hybrid, such as a DNA sequence of contiguous nucleotides linked to an RNA sequence of contiguous nucleotides, or with some regions of RNA and some regions of DNA.

As used herein, the term “RNA-mediated” in reference to RNA-mediated disorders, diseases, and/or conditions means any disease or other deleterious condition in which RNA, such as an overexpressed, underexpressed, mutant, misfolded, expanded, pathogenic, or oncogenic RNA, is known to play a role.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry: Reactions, Mechanisms, and Structure, M. B. Smith and J. March, 7^{th} Edition, John Wiley & Sons: 2013; the entire contents of each of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally, or alternatively, any substitutable nitrogen of a bridged bicyclic group is optionally substituted. Exemplary bicyclic rings include:

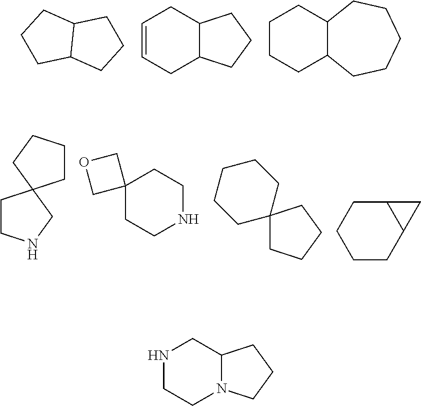

Exemplary bridged bicyclics include:

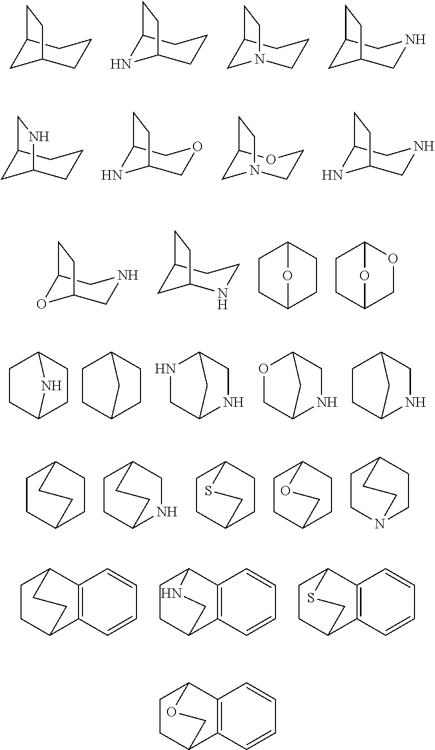

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted with a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted with a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent (“optional substituent”) at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}, —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{o} (or the ring formed by taking two independent occurrences of R^{⋅} together with their intervening atoms), are independently halogen, —(CH₂)₀₋₂R^{⋅}, -(haloR^{⋅}), —(CH₂)₀₋₂OH, —(CH₂)₀₋₂OR^{⋅}, —(CH₂)₀₋₂CH(OR^{⋅})₂; —O(haloR^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SH, —(CH₂)₀₋₂NH₂, —(CH₂)₀₋₂NHR^{⋅}, —(CH₂)₀₋₂NR^{⋅}₂, —NO₂, —SiR^{⋅}₃, —OSiR^{⋅}₃, —C(O)SR^{⋅}, —(C₁₋₄ straight or branched alkylene)C(O)OR^{⋅}, or —SSR^{⋅} wherein each R^{⋅} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(RT)S(O)₂R^{†}; wherein each Rt is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of RT are independently halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “binder” or “ligand” is defined as a compound that binds to a target RNA transcript or decay factor (e.g., nuclease) or RBP with measurable affinity. In certain embodiments, a binder has an IC₅₀ and/or binding constant of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label,” “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “RNA” (ribonucleic acid) as used herein, means a naturally-occurring or synthetic oligo- or polyribonucleotide independent of source (e.g., the RNA may be produced by a human, animal, plant, virus, or bacterium, or may be synthetic in origin), biological context (e.g., the RNA may be in the nucleus, circulating in the blood, in vitro, cell lysate, or isolated or pure form), or physical form (e.g., the RNA may be in single-, double-, or triple-stranded form (including RNA-DNA hybrids), may include epigenetic modifications, native post-transcriptional modifications, artificial modifications (e.g., obtained by chemical or in vitro modification), or other modifications, may be bound to, e.g., metal ions, small molecules, protein chaperones, or co-factors, or may be in a denatured, partially denatured, or folded state including any native or unnatural secondary or tertiary structure such as junctions (e.g., cis or trans three-way junctions (3WJ)), quadruplexes, hairpins, triplexes, hairpins, bulge loops, pseudoknots, and internal loops, etc., and any transient forms or structures adopted by the RNA). In some embodiments, the RNA is 100 or more nucleotides in length. In some embodiments, the RNA is 250 or more nucleotides in length. In some embodiments, the RNA is 350, 450, 500, 600, 750, or 1,000, 2,000, 3,000, 4,000, 5,000, 7,500, 10,000, 15,000, 25,000, 50,000, or more nucleotides in length. In some embodiments, the RNA is between 250 and 1,000 nucleotides in length. In some embodiments, the RNA is a pre-RNA, pre-miRNA, or pretranscript. In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, ceRNA, pseudo-gene, viral RNA, or bacterial RNA. The term “target RNA” as used herein, means any type of RNA having or capable of adopting a secondary or tertiary structure that is capable of binding a small molecule ligand described herein. The target RNA may be inside a cell, in a cell lysate, or in isolated form prior to contacting the small molecule.

Targeting RNA Transcripts with Compounds of the Present Invention

In one aspect, the present invention provides a method of modulating the activity of a target RNA transcript or an isoform, fragment, or mutant thereof, comprising contacting the target RNA transcript or an isoform, fragment, or mutant thereof with a disclosed compound or a pharmaceutically acceptable salt thereof that binds to the target RNA transcript or an isoform, fragment, or mutant thereof.

In another aspect, the present invention provides a method of modulating the activity of a target protein or mutant thereof, comprising contacting a corresponding target RNA transcript or an isoform, fragment, or mutant thereof with a disclosed compound or a pharmaceutically acceptable salt thereof that binds to the target RNA transcript or an isoform, fragment, or mutant thereof.

In one aspect, the present invention provides a method of decreasing the half-life or increasing degradation of a target RNA transcript or an isoform, fragment, or mutant thereof, comprising contacting the target RNA transcript or an isoform, fragment, or mutant thereof with a disclosed compound that binds to the target RNA transcript or an isoform, fragment, or mutant thereof.

In some embodiments, translation of the target RNA transcript is decreased or inhibited, e.g., by decreasing the half-life of the transcript. In some embodiments, production of the corresponding functional protein or a mutant thereof is decreased or inhibited.

In some embodiments, the administration of a compound or composition provided herein results in decrease or inhibition of the production of a functional protein or a mutant thereof. In some embodiments, the production of a functional protein or a mutant thereof is decreased by at least 10%, at least 20%, at least 30%, at least 40%, at least 25%, at least 60%, at least 70%, at least 80%, at least 90%, or is no longer produced at detectable levels.

In some embodiments, the activity of the target RNA transcript or an isoform, fragment, or mutant thereof is inhibited or decreased. In some embodiments, processing or splicing of the target RNA transcript or an isoform, fragment, or mutant thereof is inhibited.

In some embodiments, the target RNA is an mRNA, or a precursor, isoform, fragment, or mutant thereof. In some embodiments, inhibition of processing or splicing results in a decrease in levels of mature mRNA and/or protein. In some embodiments, the activity of the protein or mutant thereof is inhibited or decreased, e.g., due to a decreased level of the protein in a cell.

In some embodiments, the target RNA transcript comprises a functionally relevant fragment of a disease-causing RNA. A target RNA transcript or an isoform, fragment, or mutant thereof is “functionally relevant” if it includes at least a portion of a target RNA transcript that is ultimately transcribed and that is essential to producing a corresponding, disease-causing functional protein or mutant thereof.

In some embodiments, the target RNA transcript is a pre-mRNA, mature mRNA, or partially processed mRNA, or an isoform, fragment, or mutant thereof.

In some embodiments, the RNA transcript comprises a 5′ untranslated region (UTR).

In some embodiments, the RNA transcript comprises an open reading frame (ORF).

In some embodiments, the RNA transcript comprises a 5′ cap.

In some embodiments, the RNA transcript comprises a 3′ polyA tail (polyadenylated tail).

In some embodiments, the compound binds to a 5′ untranslated region (5′ UTR), a 3′ UTR, or an intron present in the RNA transcript.

In some embodiments, translation of the RNA transcript is reduced. In some embodiments, levels of protein encoded by the RNA transcript are decreased in a biological sample contacted with a disclosed compound or composition, such as a cell culture, or decreased in a patient treated with a disclosed compound or composition. In some embodiments, degradation of the RNA transcript is increased. In some embodiments, degradation of the RNA transcript is increased due to binding of the disclosed compound.

In one aspect, the present invention provides a method of identifying a compound that binds to a target RNA transcript or an isoform, fragment, or mutant thereof, comprising i) contacting the target RNA transcript or an isoform, fragment, or mutant thereof with a disclosed compound and ii) analyzing the results by an assay disclosed herein, optionally in combination with a computational method. In some embodiments, the method comprises the use of an SEC-MS, SPR, or DEL screen to identify the compound.

In another aspect, the present invention provides a method of treating an RNA-mediated disease, disorder, or condition (which includes any protein-mediated disease, disorder or condition) in a patient in need thereof, comprising administering to the patient an effective amount of a disclosed compound or a pharmaceutically acceptable salt thereof. In some embodiments, the disease, disorder, or condition is a proliferative disorder, such as a cancer.

A variety of RNA transcripts are appropriate as target RNA transcripts for use in the present invention. In some embodiments, the target RNA transcript is selected from one of those in Table A, Table B, Table C, or Table D below, or a precursor, isoform, unspliced isoform, splicing intermediate, fragment, or mutant thereof.

In some embodiments, the target RNA transcript is single-stranded. In some embodiments, the target RNA transcript is double-stranded or partially double-stranded. In some embodiments, the target RNA is a pair of nucleic acids engaged in an interaction, such as a miRNA-mRNA hybridized (or partially hybridized) pair. In some embodiments, the target RNA comprises one, two, or more miRNAs bound to an mRNA. In some embodiments, the target RNA is an mRNA, miRNA, premiRNA, or a viral or fungal RNA.

In some embodiments, the target RNA transcript includes structural features such as at least some intramolecular base pairing, a junction (e.g., cis or trans three-way junctions (3WJ)), quadruplex, hairpin, triplex, bulge loop, pseudoknot, or internal loop, etc., and any transient forms or structures adopted by the nucleic acid. In some embodiments, the target RNA transcript includes a bound protein, such as a chaperone, RNA-binding protein (RBP), or other nucleic acid-binding protein.

Target RNA transcripts of various lengths are target RNA transcripts within the scope of the present invention. For example, the target RNA may be from 20-10,000 nucleotides in length. In some embodiments, the target RNA is a relatively short sequence of, e.g., less than 250, less than 100, or less than 50 nucleotides in length. In some embodiments, the target RNA is 100 or more nucleotides in length. In some embodiments, the target RNA is 250 or more nucleotides in length. In some embodiments, the target RNA is up to about 350, 450, 500, 600, 750, or 1,000, 2,000, 3,000, 4,000, 5,000, 7,500, 10,000, 15,000, 25,000, 50,000, or more than 50,000 nucleotides in length. In some embodiments, the target RNA is between about 30 and about 500 nucleotides in length. In some embodiments, the target RNA is between about 250 and about 1,000 nucleotides in length. In some embodiments, the target RNA is between about 20-50, 30-60, 40-70, 50-80, 20-100, 30-100, 40-100, 50-100, 20-200, 30-200, 40-200, 50-200, 20-300, 50-300, 75-300, 100-300, 20-400, 50-400, 100-400, 200-400, 20-500, 50-500, 100-500, 250-500, 20-750, 50-750, 100-750, 250-750, 500-750, 20-1,000, 100-1,000, 250-1,000, 500-1,000, 20-2,000, 100-2,000, 500-2,000, 1,000-2,000, 20-5,000, 100-5,000, 1,000-5,000, 20-10,000, 100-10,000, 1,000-10,000, or 20-25,000 nucleotides in length.

Where the target or other referenced nucleic acid is an RNA, “nucleotides” refers to ribonucleotides. Where the target or other referenced nucleic acid is DNA, “nucleotides” refers to 2′-deoxyribonucleotides. In some embodiments, a target RNA comprises one or more nucleotide analogs (modified nucleotides) as defined herein and as known in the art.

In some embodiments, the target RNA is a pre-mRNA, pre-miRNA, pretranscript, partially spliced mRNA, fully spliced mRNA, fully spliced and partially processed mRNA, or a mature mRNA (i.e., fully spliced and processed mRNA).

In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, rRNA, ceRNA, or pseudo-gene, wherein each of the foregoing may be selected from a human or non-human RNA, such as viral RNA, fungal RNA, or bacterial RNA.

Targeting mRNA

In some embodiments, the target RNA transcript is an mRNA or a precursor to a mature mRNA; or an isoform, fragment, or mutant thereof. Within mRNAs, noncoding regions can affect the level of mRNA and protein expression. Briefly, these include internal ribosome entry sites (IRES) and upstream open reading frames (uORF) that affect translation efficiency, intronic sequences that affect splicing efficiency and alternative splicing patterns, 3′ UTR sequences that affect mRNA and protein localization, and elements that control mRNA decay and half-life. Therapeutic modulation of these RNA elements can have beneficial effects. Also, mRNAs may contain expansions of simple repeat sequences such as trinucleotide repeats. These repeat expansion containing RNAs can be toxic and have been observed to drive disease pathology, particularly in certain neurological and musculoskeletal diseases (see Gatchel & Zoghbi, Nature Rev. Gen. 2005, 6, 743-755). Accordingly, in some embodiments, the present invention provides a method of degrading an mRNA that contains a toxic repeat expansion, or an isoform, fragment, or mutant thereof, comprising contacting the mRNA with a disclosed compound. The present invention further provides a method of treating a disease, disorder, or condition mediated by an mRNA that contains a toxic repeat expansion, or an isoform, fragment, or mutant thereof.

Additionally, in some embodiments, the expression of a target mRNA and its translation products is modulated by targeting noncoding sequences and structures in the 5′ and 3′ UTRs. For instance, RNA structures in the 5′ UTR can affect translational efficiency. RNA structures such as hairpins in the 5′ UTR have been shown to affect translation. In general, RNA structures are believed to play a critical role in translation of mRNA. One example of these are internal ribosome entry sites (IRES), which can affect the level of translation of the main open reading frame (Komar and Hatzoglou, Frontiers Oncol. 5:233, 2015; Weingarten-Gabbay et al., Science 351, 4939, 2016; Calvo et al., Proc. Natl. Acad. Sci. USA 106:7507-7512; Le Quesne et al., J Pathol. 220:140-151, 2010; Barbosa et al., PLOS Genetics 9:e10035529, 2013). Small molecules targeting these RNAs could be used to modulate specific protein levels for therapeutic benefit. In some embodiments, the small molecule rSM binding site is a 5′ UTR, internal ribosome entry site, or upstream open reading frame.

### Non-Coding RNA Transcripts

Non-coding RNAs regulate cellular biology directly through function of RNA structures (e.g., ribonucleoproteins) as well as via regulating protein expression. These ncRNAs include (but are not limited to) miRNA, lncRNA, lincRNA, snoRNA, snRNA, scaRNA, piRNA, ceRNA, and pseudo-genes. Drugs that intervene at this level have the potential of modulating any cellular process.

In some embodiments, the target RNA transcript is an RNA that is transcribed but not translated into protein, termed “non-coding RNA” or “ncRNA.” Non-coding RNA is highly conserved, and the many varieties of non-coding RNA play a wide range of regulatory functions. The term “non-coding RNA,” as used herein, includes but is not limited to micro-RNA (miRNA), long non-coding RNA (lncRNA), long intergenic non-coding RNA (lincRNA), Piwi-interacting RNA (piRNA), competing endogenous RNA (ceRNA), and pseudo-genes. Each of these sub-categories of non-coding RNA offers a large number of RNA targets with significant therapeutic potential. Accordingly, in some embodiments, the present invention provides methods of treating a disease mediated by a non-coding transcript. In some embodiments, the disease is caused by a lncRNA, lincRNA, ceRNA, or pseudo-gene. In another aspect, the present invention provides a method of producing a small molecule that modulates the activity of a target non-coding transcript to treat a disease or disorder, comprising the steps of: screening one or more disclosed compounds for binding to or degradation of the target non-coding transcript; and analyzing the results by an RNA binding assay disclosed herein. In some embodiments, the target non-coding transcript is a lncRNA, lincRNA, ceRNA, or pseudo-gene.

In some embodiments, the target RNA transcript is an miRNA. miRNA are short double-strand RNAs that regulate gene expression (see Elliott & Ladomery, Molecular Biology of RNA, 2^{nd} Ed.). Each miRNA can affect the expression of many human genes. There are nearly 2,000 miRNAs in humans. These RNAs regulate many biological processes, including cell differentiation, cell fate, motility, survival, and function. miRNA expression levels vary between different tissues, cell types, and disease settings. They are frequently aberrantly expressed in tumors versus normal tissue, and their activity may play significant roles in cancer (for reviews, see Croce, Nature Rev. Genet. 10:704-714, 2009; Dykxhoorn Cancer Res. 70:6401-6406, 2010). miRNAs have been shown to regulate oncogenes and tumor suppressors and themselves can act as oncogenes or tumor suppressors. Some have been shown to promote epithelial-mesenchymal transition (EMT) and cancer cell invasiveness and metastasis. In the case of oncogenic miRNAs, their inhibition could be an effective anti-cancer treatment. Accordingly, in one aspect, the present invention provides a method of producing a small molecule that modulates the activity of a target miRNA to treat a disease or disorder, comprising the steps of: screening one or more disclosed compounds for binding to or degradation of the target miRNA; and analyzing the results by an RNA binding assay disclosed herein. In some embodiments, the miRNA regulates an oncogene or tumor suppressor, or acts as an oncogene or tumor suppressor. In some embodiments, the disease is cancer. In some embodiments, the cancer is a solid tumor.

Beyond oncology, miRNAs play roles in many other diseases including cardiovascular and metabolic diseases (Quiant and Olson, J. Clin. Invest. 123:11-18, 2013; Olson, Science Trans. Med. 6: 239ps3, 2014; Baffy, J. Clin. Med. 4:1977-1988, 2015).

Many mature miRNAs are relatively short in length and thus may lack sufficient folded, three-dimensional structure to be targeted by small molecules. However, it is believed that the levels of such miRNA could be reduced by small molecules that bind the primary transcript or the pre-miRNA to block the biogenesis of the mature miRNA. Accordingly, in some embodiments of the methods described above, the target miRNA is a primary transcript or pre-miRNA whose corresponding mature miRNA affects an oncogene or tumor suppressor, or which affects the levels or activity of a disease-causing RNA transcript or protein.

In some embodiments, the target RNA transcript is an lncRNA. lncRNA are RNAs of over 200 nucleotides (nt) that do not encode proteins (see Rinn & Chang, Ann. Rev. Biochem. 2012, 81, 145-166; (for reviews, see Morris and Mattick, Nature Reviews Genetics 15:423-437, 2014; Mattick and Rinn, Nature Structural & Mol. Biol. 22:5-7, 2015; Iyer et al., Nature Genetics 47(: 199-208, 2015)). They can affect the expression of the protein-encoding mRNAs at the level of transcription, splicing and mRNA decay. Considerable research has shown that lncRNA can regulate transcription by recruiting epigenetic regulators that increase or decrease transcription by altering chromatin structure (e.g., Holoch and Moazed, Nature Reviews Genetics 16:71-84, 2015). lncRNAs are associated with human diseases including cancer, inflammatory diseases, neurological diseases and cardiovascular disease (for instance, Presner and Chinnaiyan, Cancer Discovery 1:391-407, 2011; Johnson, Neurobiology of Disease 46:245-254, 2012; Gutscher and Diederichs, RNA Biology 9:703-719, 2012; Kumar et al., PLOS Genetics 9:e1003201, 2013; van de Vondervoort et al., Frontiers in Molecular Neuroscience, 2013; Li et al., Int. J. Mol. Sci. 14:18790-18808, 2013). In general, lncRNA are expressed at a lower level relative to mRNAs. Many lncRNAs are physically associated with chromatin (Werner et al., Cell Reports 12, 1-10, 2015) and are transcribed in close proximity to protein-encoding genes. They often remain physically associated at their site of transcription and act locally, in cis, to regulate the expression of a neighboring mRNA.

lncRNAs regulate the expression of protein-encoding genes, acting at multiple different levels to affect transcription, alternative splicing and mRNA decay. For example, lncRNA has been shown to bind to the epigenetic regulator PRC2 to promote its recruitment to genes whose transcription is then repressed via chromatin modification. lncRNA may form complex structures that mediate their association with various regulatory proteins. A small molecule that binds to these lncRNA structures could be used to modulate the expression of genes that are normally regulated by an individual lncRNA.

### Targeting Toxic RNA (Repeat RNA)

Simple repeats in mRNA often are associated with human disease. These are often, but not exclusively, repeats of three nucleotides such as CAG (“triplet repeats”) (for reviews, see Gatchel and Zoghbi, Nature Reviews Genetics 6:743-755, 2005; Krzyzosiak et al., Nucleic Acids Res. 40:11-26, 2012; Budworth and McMurray, Methods Mol. Biol. 1010:3-17, 2013, hereby incorporated by reference). Triplet repeats are abundant in the human genome, and they tend to undergo expansion over generations. Approximately 40 human diseases are associated with the expansion of repeat sequences. Diseases caused by triplet expansions are known as Triplet Repeat Expansion Diseases (TRED). Healthy individuals have a variable number of triplet repeats, but there is a threshold beyond which a higher repeat number causes disease. The threshold varies in different disorders. The triplet repeat can be unstable. As the gene is inherited, the number of repeats may increase, and the condition may be more severe or have an earlier onset from generation to generation. When an individual has a number of repeats in the normal range, it is not expected to expand when passed to the next generation. When the repeat number is in the premutation range (a normal, but unstable repeat number), then the repeats may or may not expand upon transmission to the next generation. Normal individuals who carry a premutation do not have the condition but are at risk of having a child who has inherited a triplet repeat in the full mutation range and who will be affected. TREDs can be autosomal dominant, autosomal recessive or X-linked. The more common triplet repeat disorders are autosomal dominant.

The repeats can be in the coding or noncoding portions of the mRNA. In the case of repeats within noncoding regions, the repeats may lie in the 5′ UTR, introns, or 3′ UTR sequences. Some examples of diseases caused by repeat sequences within coding regions are shown in Table A.

TABLE A
Repeat Expansion Diseases in Which the
Repeat Resides in the Coding Regions
of mRNA

| | | | Normal | Disease |
|---|---|---|---|---|
| | | | repeat | repeat |
| Disease | Gene | Repeat | number | number |
| HD | HTT | CAG | 6-35 | 36-250 |
| | | | (SEQ ID | (SEQ ID |
| | | | NO: 16) | NO: 23) |
| DRPLA | ATN1 | CAG | 6-35 | 49-88 |
| | | | (SEQ ID | (SEQ ID |
| | | | NO: 16) | NO: 24) |
| SBMA | AR | CAG | 9-36 | 38-62 |
| | | | (SEQ ID | (SEQ ID |
| | | | NO: 17) | NO: 25) |
| SCA1 | ATXN1 | CAG | 6-35 | 49-88 |
| | | | (SEQ ID | (SEQ ID |
| | | | NO: 16) | NO: 24) |
| SCA2 | ATXN2 | CAG | 14-32 | 33-77 |
| | | | (SEQ ID | (SEQ ID |
| | | | NO: 18) | NO: 26) |
| SCA3 | ATXN3 | CAG | 12-40 | 55-86 |
| | | | (SEQ ID | (SEQ ID |
| | | | NO: 19) | NO: 27) |
| SCA6 | CACNA1A | CAG | 4-18 | 21-30 |
| | | | (SEQ ID | (SEQ ID |
| | | | NO: 20) | NO: 28) |
| SCA7 | ATXN7 | CAG | 7-17 | 38-120 |
| | | | (SEQ ID | (SEQ ID |
| | | | NO: 21) | NO: 29) |
| SCA17 | TBP | CAG | 25-42 | 47-63 |
| | | | (SEQ ID | (SEQ ID |
| | | | NO: 22) | NO: 30) |

In some embodiments, the target RNA is one of those listed in Table A, or a precursor, isoform, fragment, or mutant thereof.

Some examples of diseases caused by repeat sequences within noncoding regions of mRNA are shown in Table B.

TABLE B
Repeat Expansion Diseases in Which the
Repeat Resides in the Noncoding
Regions of mRNA

| | | | | Normal | Disease |
|---|---|---|---|---|---|
| | | | Repeat | repeat | repeat |
| Disease | Gene | Repeat | location | number | number |
| Fragile | FMR1 | CGG | 5′ UTR | 6-53 | ≥230 |
| X | | | | (SEQ ID | |
| | | | | NO: 31) | |
| DM1 | DMPK | CTG | 3′ UTR | 5-37 | ≥50 |
| | | | | (SEQ ID | |
| | | | | NO: 32) | |
| FRDA | FXN | GAA | Intron | 7-34 | ≥100 |
| | | | | (SEQ ID | |
| | | | | NO: 33) | |
| SCA8 | ATXN8 | CTG | Noncoding | 16-37 | 110-250 |
| | | | antisense | (SEQ ID | (SEQ ID |
| | | | | NO: 34) | NO: 37) |
| SCA10 | ATXN10 | ATTCT | Intron | 9-32 | 800-4500 |
| | | | | (SEQ ID | (SEQ ID |
| | | | | NO: 35) | NO: 38) |
| SCA12 | PPP2R2B | CAG | 5′ UTR | 7-28 | 66-78 |
| | | | | (SEQ ID | (SEQ ID |
| | | | | NO: 36) | NO: 39) |
| C9FTD/ | C9orf72 | GGGGCC | Intron | ~30 | 100s |
| ALS | | | | | |

In some embodiments, the target RNA is one of those listed in Table B, or a precursor, isoform, fragment, or mutant thereof.

The toxicity that results from the repeat sequence can be direct consequence of the action of the toxic RNA itself, or, in cases in which the repeat expansion is in the coding sequence, due to the toxicity of the RNA and/or the aberrant protein. The repeat expansion RNA can act by sequestering critical RNA-binding proteins (RBP) into foci. One example of a sequestered RBP is the Muscleblind family protein MBNL1. Sequestration of RBPs leads to defects in splicing as well as defects in nuclear-cytoplasmic transport of RNA and proteins. Sequestration of RBPs also can affect miRNA biogenesis. These perturbations in RNA biology can profoundly affect neuronal function and survival, leading to a variety of neurological diseases.

Repeat sequences in RNA form secondary and tertiary structures that bind RBPs and affect normal RNA biology. One specific example disease is myotonic dystrophy (DM1; dystrophia myotonica), a common inherited form of muscle disease characterized by muscle weakness and slow relaxation of the muscles after contraction (Machuca-Tzili et al., Muscle Nerve 32:1-18, 2005, hereby incorporated by reference). It is caused by a CUG expansion in the 3′ UTR of the dystrophia myotonica protein kinase (DMPK) gene. This repeat-containing RNA causes the misregulation of alternative splicing of several developmentally regulated transcripts through effects on the splicing regulators MBNL1 and the CUG repeat binding protein (CELF1) (Wheeler et al., Science 325:336-339, 2009, hereby incorporated by reference). Small molecules that bind the CUG repeat within the DMPK transcript would alter the RNA structure and prevent focus formation and alleviate the effects on these spicing regulators. Fragile X Syndrome (FXS), the most common inherited form of mental retardation, is the consequence of a CGG repeat expansion within the 5′ UTR of the FMR1 gene (Lozano et al., Intractable Rare Dis. Res. 3:134-146, 2014, hereby incorporated by reference). FMRP is critical for the regulation of translation of many mRNAs and for protein trafficking, and it is an essential protein for synaptic development and neural plasticity. Thus, its deficiency leads to neuropathology. A small molecule targeting this CGG repeat RNA may alleviate the suppression of FMR1 mRNA and FMRP protein expression. Another TRED having a very high unmet medical need is Huntington's disease (HD). HD is a progressive neurological disorder with motor, cognitive, and psychiatric changes (Zuccato et al., Physiol Rei. 90:905-981, 2010, hereby incorporated by reference). It is characterized as a poly-glutamine or polyQ disorder since the CAG repeat within the coding sequence of the HTT gene leads to a protein having a poly-glutamine repeat that appears to have detrimental effects on transcription, vesicle trafficking, mitochondrial function, and proteasome activity. However, the HTT CAG repeat RNA itself also demonstrates toxicity, including the sequestration of MBNL1 protein into nuclear inclusions. One other specific example is the GGGGCC repeat expansion in the C9orf72 (chromosome 9 open reading frame 72) gene that is prevalent in both familial frontotemporal dementia (FTD) and amyotrophic lateral sclerosis (ALS) (Ling et al., Neuron 79:416-438, 2013; Haeusler et al., Nature 507:195-200, 2014, hereby incorporated by reference). The repeat RNA structures form nuclear foci that sequester critical RNA binding proteins. The GGGGCC repeat RNA also binds and sequesters RanGAP1 to impair nucleocytoplasmic transport of RNA and proteins (Zhang et al., Nature 525:56-61, 2015, hereby incorporated by reference). Selectively targeting any of these repeat expansion RNAs could add therapeutic benefit in these neurological diseases.

The present invention includes a method of treating a disease or disorder wherein aberrant RNAs themselves cause pathogenic effects, rather than acting through the agency of protein expression or regulation of protein expression. In some embodiments, the target RNA is a repeat RNA, such as those described herein or in Table A or Table B. In some embodiments, the repeat RNA mediates or is implicated in a repeat expansion disease in which the repeat resides in the coding regions of mRNA. In some embodiments, the disease or disorder is a repeat expansion disease in which the repeat resides in the noncoding regions of mRNA. In some embodiments, the disease or disorder is selected from Huntington's disease (HD), dentatorubral-pallidoluysian atrophy (DRPLA), spinal-bulbar muscular atrophy (SBMA), or a spinocerebellar ataxia (SCA) selected from SCA1, SCA2, SCA3, SCA6, SCA7, or SCA17. In some embodiments, the disease or disorder is selected from Fragile X Syndrome, myotonic dystrophy (DM1 or dystrophia myotonica), Friedreich's Ataxia (FRDA), a spinocerebellar ataxia (SCA) selected from SCA8, SCA10, or SCA12, or C9FTD (amyotrophic lateral sclerosis or ALS).

In some embodiments, the disease is amyotrophic lateral sclerosis (ALS), Huntington's disease (HD), frontotemporal dementia (FTD), myotonic dystrophy (DM1 or dystrophia myotonica), or Fragile X Syndrome.

Also provided is a method of producing a small molecule that modulates the activity of a target repeat expansion RNA to treat a disease or disorder, comprising the steps of: screening one or more disclosed compounds for binding to the target repeat expansion RNA; and analyzing the results by an RNA binding assay disclosed herein. In some embodiments, the repeat expansion RNA causes a disease or disorder selected from HD, DRPLA, SBMA, SCA1, SCA2, SCA3, SCA6, SCA7, or SCA17. In some embodiments, the disease or disorder is selected from Fragile X Syndrome, DM1, FRDA, SCA8, SCA10, SCA12, or C9FTD.

### Target RNAs and Diseases/Conditions

An association is known to exist between a large number of RNAs and diseases or conditions, some of which are shown below in Table C or Table D. Accordingly, in some embodiments of the methods described above, the target RNA transcript is selected from one of those in Table C or Table D. In some embodiments, the target RNA mediates or is implicated in a disease or disorder selected from one of those in Table C or Table D. Accordingly, the present invention further provides a method of treating a disease, disorder, or condition selected from one of those in Table C or Table D, comprising the step of administering to a patient in need thereof an effective amount of a disclosed compound. In some embodiments, the method up- or down-regulates the target RNA transcript as shown in the “UP/DOWN REGULATION DESIRABLE?” column in Table C or Table D, below, thus treating the disease, disorder, or condition.

TABLE C
Exemplary Target RNA Transcripts and Associated Diseases

| | | UP/DOWN | | |
|---|---|---|---|---|
| | | REGULATION | Therapeutic | |
| GENE | CLASS | DESIRABLE? | Area | INDICATION(S) |
| MYC | TF | down | Onco | cancer |
| STAT3 | TF | down | Onco | cancer |
| C9orf72 | TRED | down | Neuro | ALS, FTD |
| FOXP3 | TF | down | I&I, I-O | Immuno-oncology; I&I |
| MIR155 | miRNA | down | Onco, I&I, | ALS, fibrosis, cancer |
| | | | Neuro | |
| APOC3 | apoprotein | down | Cardio | chylomicronemia syndrome |
| JUN | TF | down | I&I | I&I |
| RSV | genomic | down | Viral | RSV |
| KRAS | TF | down | Onco | cancer |
| BCL2L1 | IAP | down | Onco | cancer |
| HIF1A | TF | down | Onco | cancer |
| SMARCA2 | helicase | down | Onco | cancer |
| SNCA | | down | Neuro | PD |
| CCNE1 | cyclin | down | Onco | cancer |
| FOXM1 | TA | down | Onco | cancer |
| MYB | TF | down | Onco | cancer |
| PTPN11 | phosphatase | down | Onco, I&I | cancer, SLE |
| CD40LG | TNF | down | I&I | inflammation |
| NFE2L2 | TF | up | I&I | multiple sclerosis |
| RORC | NHR | down | I&I | I&I |
| ZIKV | genomic | down | Viral | ZIKV |
| DENV | genomic | down | Viral | DENV |
| AR | NHR | down | Onco | prostate cancer |
| ASGR1 | | down | Cardio | CVD (cardiovascular disease) |
| BCL2 | IAP | down | Onco | cancer |
| BDNF | NF | up | Neuro | Huntington's Disease |
| BRD4 | epi | down | Onco | cancer |
| CD40 | TNF | down | I&I | Immuno-oncology |
| CD47 | Ig | down | I&I, I-O | Immuno-oncology |
| CTLA4 | Ig | down | I&I, I-O | Immuno-oncology; I&I |
| CTNNB1 | adhesion | down | Onco | Cancer |
| DMPK | TRED | down | Neuro | Myotonic dystrophy type 1 (DM1) |
| EIF4E | IF | down | Onco | Cancer |
| FOXA1 | TA | down | Onco | Cancer |
| GATA3 | TF | down | Onco | Cancer |
| IKZF1 | TF | down | Onco | cancer |
| IKZF3 | TF | down | Onco | cancer |
| IL17A | IL | down | I&I | inflammatory & autoimmune |
| | | | | diseases |
| IL23A | IL | down | I&I | inflammatory & autoimmune |
| | | | | diseases |
| IL6 | IL | down | I&I | rheumatoid arthritis |
| ITGA1 | integrin | down | I&I | RA |
| ITGA5 | integrin | down | Onco | solid tumors |
| ITGAE | integrin | down | I&I | UC, Crohns |
| ITGB2, | integrin | down | I&I | psoriasis |
| ITGAL | | | | |
| ITGB7 | integrin | down | I&I | UC, Crohns |
| MAPT | cytoskeleton | down | Neuro | Alzheimer's disease |
| MAX | TF | down | Onco | cancer |
| MDM2 | E3 | down | Onco | cancer |
| MDM4 | E3 | down | Onco | cancer |
| MIR21 | miRNA | down | Onco | cancer |
| NR4A2 | TF | down | Neuro | PD |
| PTEN | phosphatase | up | Onco | cancer |
| PTPN1 | phosphatase | down | Metab | Type 2 diabetes |
| RUNX1 | TF | down | Onco | cancer |
| SIRPA | glycoprotein | down | I&I, I-O | Immuno-oncology |
| SMAD7 | TGF | down | I&I | IBD |
| SOX2 | TF | down | Onco | cancer |
| STAT5A | TF | down | Onco | cancer |
| TERT | telomerase | down | Onco | cancer |
| TGFB1 | TGF | down | Fibrosis | fibrosis |
| TNF | TNF | down | I&I | inflammatory disease |
| TNFRSF11A | TNF | down | | osteoporosis |
| TNFSF11 | TNF | down | | osteoporosis |
| TWIST1 | TF | down | Onco | cancer |
| WNT1 | | | Onco | cancer |
| HepB | | down | Viral | HepB |
| influenza | | down | Viral | influenza |
| DGAT2 | transferase | down | | NASH |
| DNMT3 | DNMT | down | Onco | cancer |
| ERBB3 | pseudokinase | down | Onco | cancer |
| FBXW7 | F-box (E3) | down | Onco | cancer |
| FMR1 | TRED | down | Neuro | Fragile X Syndrome; FTXAS |
| FOS | TF | down | | |
| FXN | TRED | down | Neuro | Friedreich's Ataxia |
| IRAK3 | pseudokinase | down | I&I | I&I |
| MECP2 | TF | up/down | Genetic | Rett Syndrome |
| | | | Disease | |
| MIR17HG | miRNA | down | Onco | cancer |
| NF1 | | down | | neurofibromatosis |
| ORAI1 | ion channel | down | I&I | I&I |
| PCSK9 | convertase | down | Cardio | hypercholesterolemia |
| PSMB8 | protease | down | I&I | I&I |
| SKP2 | F-box (E3) | down | Onco | cancer |
| USP1 | protease | down | Onco | cancer |
| USP7 | protease | down | Onco | cancer |
| HIF1A | TF | up | | wound repair & regeneration |
| HOTAIR | IncRNA | down | Onco | cancer |
| IKBKG | | down | I&I | I&I |
| IKK2 | kinase | down | I&I | I&I |
| MALAT1 | IncRNA | down | Onco | cancer |
| PRMT5 | KMT | down | Onco | cancer |
| BCL6 | IAP | down | Onco | cancer |
| GRN | | down | Neuro | neurological diseases |
| ABCA1 | transporter | | Cardio | coronary artery disease |
| ABCB11 | transporter | | | Primary Biliary Sclerosis |
| ABCB4 | transporter | | | Primary Biliary Sclerosis |
| ABCG5 | transporter | | Cardio | coronary artery disease |
| ABCG8 | transporter | | Cardio | coronary artery disease |
| ADIPOQ | hormone | up | Metab | diabetes; obesity; metabolic |
| | | | | syndrome |
| APOA1 | | | Cardio | hypercholesterolemia |
| APOA5 | | | Cardio | hypercholesterolemia |
| ATPA2 | Ca ATPase | up | Genetic | congestive heart failure |
| | | | Disease | |
| ATXN1 | TRED | | Neuro | spinocerebellar ataxia 1 |
| ATXN10 | TRED | down | Neuro | spinocerebellar ataxia 10 |
| ATXN2 | TRED | | Neuro | spinocerebellar ataxia 2 |
| ATXN3 | TRED | | Neuro | spinocerebellar ataxia 3 |
| ATXN7 | TRED | | Neuro | spinocerebellar ataxia 7 |
| ATXN8 | TRED | | Neuro | spinocerebellar ataxia 8 |
| BACE1 | protease | down | Neuro | Alzheimer's disease |
| BIRC2 | IAP | down | Onco | cancer |
| BIRC3 | IAP | down | Onco | cancer |
| BIRC5 | IAP | down | Onco | cancer |
| BRCA1 | DNA repair | up | Onco | cancer |
| CACNA1A | ion channel | | Neuro | episodic ataxia type 2 |
| CD247 | TCR | | I&I | I&I |
| CD274 | | down | I-O | Immuno-oncology |
| CETP | transfer | down | | cardiovascular |
| CFH | complement | | | macular degeneration |
| CFTR | ion channel | up | Genetic | Cystic Fibrosis |
| | | | Disease | |
| CNBP | TRED | down | Neuro | Myotonic dystrophy type 2 (DM2) |
| CNTF | NF | | | macular degeneration |
| DIO2 | deiodinase | | Metab | dyslipidemia |
| DMD | cytoskeleton | | Neuro | Duchenne Muscular Dystrophy; |
| | | | | Becker's MD |
| F7 | protease | up | Hematology | hemophilia |
| F8 | protease | up | Hematology | hemophilia |
| F9 | protease | up | Hematology | hemophilia |
| FGF3 | | down | Genetic | achondroplasia |
| | | | Disease | |
| HAMP | | down | Genetic | thalassemia; hereditary |
| | | | Disease | hemochromatosis |
| HAVCR2 | | down | I&I, I-O | inflammatory diseases; Immuno- |
| | | | | oncology |
| HBG1, | hemoglobin | up | Hematology | sickle cell anemia; beta-thalassemia |
| HBG2 | | | | |
| HIF1AN | | | Onco | cancer |
| IDH1 | dehydrogenase | down | Onco | cancer |
| IL1 | IL | down | I&I | rheumatoid arthritis |
| IRAK4 | kinase | down | I&I | I&I |
| IRF5 | TF | | I-O | Immuno-oncology |
| LAMA1 | ECM | | Genetic | Merosin-deficient congenital MD |
| | | | Disease | (MDCA1) |
| LARGE1 | | | Genetic | Muscular Dystroglycanopathy Type |
| | | | Disease | B,6 |
| LINGO1 | | down | Neuro | neurodegeneration |
| MBNL1 | splice factor | | Neuro | Myotonic Dystrophy |
| MCL1 | IAP | down | Onco | cancer |
| MERTK | kinase | | I&I | Lupus |
| METAP2 | peptidase | down | Onco, I&I | cancer, obesity, autoimmune |
| MTOR | kinase | | Onco | cancer |
| NANOG | TF | | Neuro | neurological diseases |
| NF2 | | | | neurofibromatosis |
| NSD-3 | KMT | down | Onco | cancer |
| PAH | hydroxylase | | Genetic | phenylketonuria |
| | | | Disease | |
| PCSK6 | convertase | up | Cardio | hypertension |
| PDCD1 | | | I-O | Immuno-oncology |
| PDK1, | kinase | | | polycystic kidney disease |
| PDK2 | | | | |
| PDX1 | TF | | Metab | diabetes |
| PPARGC1A | PPAR | | Neuro | Neurological diseases; obesity |
| PRKAA1 | kinase | | Metab | diabetes |
| PRKAB1 | kinase | | Metab | diabetes |
| PRKAG1 | kinase | | Metab | diabetes |
| RTN4 | | down | Neuro | neurodegeneration |
| RTN4R | | down | Neuro | neurodegeneration |
| SCARB1 | HDL receptor | | Cardio | coronary artery disease |
| SIRT6 | KDAC | down | Onco | cancer |
| SMN2 | | up | Neuro | Spinal Muscular Atrophy |
| SMURF2 | | down | | |
| SORT1 | glycoprotein | | Cardio | coronary artery disease |
| SSPN | cytoskeleton | | Genetic | Duchenne's MD |
| | | | Disease | |
| TBX21 | | | I-O | Immuno-oncology |
| THRB | NHR | | | dyslipidemia; NASH; NAFLD |
| TNFAIP3 | | | I&I | inflammatory disease; liver failure; |
| | | | | liver transplant |
| TRIB1 | pseudokinase | | Cardio | coronary artery disease |
| TTR | | down | Genetic | amyloidosis |
| | | | Disease | |
| UTRN | cytoskeleton | | Genetic | Duchenne Muscular Dystrophy |
| | | | Disease | |
| XIAP | IAP | down | Onco | cancer |
| RAGE | | | | |
| ANGPTL3 | | | | |

TABLE D
Additional Target RNA Transcripts

| | | | UP/DOWN | | |
|---|---|---|---|---|---|
| | COMMON | | REGULATION | | |
| GENE | NAME | CLASS | DESIRABLE? | TA | INDICATION(S) |
| CTSL | cathepsin L | protease | up | neuro | PD |
| AR | AR-V7 | NHR | down | cancer | CRPC |
| JMJD6 | JMJD6 | HDM | down | cancer | GBM |
| DNMT1 | DNMT1 | Me-transferase | down | cancer | GBM |
| ASGR1 | ASGR1 | ASG receptor | down | CVD | CVD |
| NAMPT | NAMPT | transferase | down | cancer | various |
| | IRE | | | | |
| ARID1B | ARID1B | | | | |
| SOX10 | SOX10 | | | | |
| HNF1B | TCF2 | | | | |
| PTPN2 | PTPN2 | | | | |
| NLGN3 | NLGN3 | | | | |
| | ETS | | | | |

### 3. General Methods of Providing the Present Compounds

The compounds of this invention may be prepared or isolated in general by synthetic and/or semi-synthetic methods known to those skilled in the art for analogous compounds and by methods described in detail in the Examples and Figures, herein.

In the schemes and chemical reactions depicted in the detailed description, Examples, and Figures, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, and Protective Groups in Organic Synthesis, P. G. M. Wuts, 5^{th} edition, John Wiley & Sons, 2014, the entirety of each of which is hereby incorporated herein by reference.

As used herein, the phrase “leaving group” (LG) includes, but is not limited to, halogens (e.g., fluoride, chloride, bromide, iodide), sulfonates (e.g., mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protective Groups in Organic Synthesis, P. G. M. Wuts, 5^{th} edition, John Wiley & Sons, 2014, and Philip Kocienski, in Protecting Groups, Georg Thieme Verlag Stuttgart, New York, 1994, the entireties of which are incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formate, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protective Groups in Organic Synthesis, P. G. M. Wuts, 5^{th} edition, John Wiley & Sons, 2014, and Philip Kocienski, in Protecting Groups, Georg Thieme Verlag Stuttgart, New York, 1994, the entireties of which are incorporated herein by reference. Suitable amino protecting groups include, but are not limited to, aralkylamines, carbamates, cyclic imides, allyl amines, amides, and the like. Examples of such groups include t-butyloxycarbonyl (Boc), ethyloxycarbonyl, methyloxycarbonyl, trichloroethyloxycarbonyl, allyloxycarbonyl (Alloc), benzyloxocarbonyl (Cbz), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See, for example, March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, the entirety of each of which is incorporated herein by reference. Such interconversions may require one or more of the aforementioned techniques, and certain methods for synthesizing compounds of the invention are described below.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. Such groups and transformations are described in detail in March's Advanced Organic Chemistry: Reactions, Mechanisms, and Structure, M. B. Smith and J. March, 7^{th} Edition, John Wiley & Sons, 2013, Comprehensive Organic Transformations, R. C. Larock, 3^{rd} Edition, John Wiley & Sons, 2018, and Protective Groups in Organic Synthesis, P. G. M. Wuts, 5^{th} edition, John Wiley & Sons, 2014, the entirety of each of which is hereby incorporated herein by reference. Such interconversions may require one or more of the aforementioned techniques, and certain methods for synthesizing compounds of the invention are described below in the Exemplification and Figures.

### EXEMPLIFICATION

As depicted in the Examples below, exemplary compounds are prepared according to the following general procedures and used in biological assays and other procedures described generally herein. It will be appreciated that, although the general methods depict the synthesis of certain compounds of the present invention, the following general methods, and other methods known to one of ordinary skill in the art, can be applied to all compounds and subclasses and species of each of these compounds, as described herein. Similarly, assays and other analyses can be adapted according to the knowledge of one of ordinary skill in the art.

### Example 1: General Synthesis of Compounds I-1, I-2, I-3, I-4, I-5, I-6, and I-7 General Synthesis of Intermediates Used in the Synthesis of Compounds I-1, I-2, I-3, I-4, I-5, I-6 and I-7

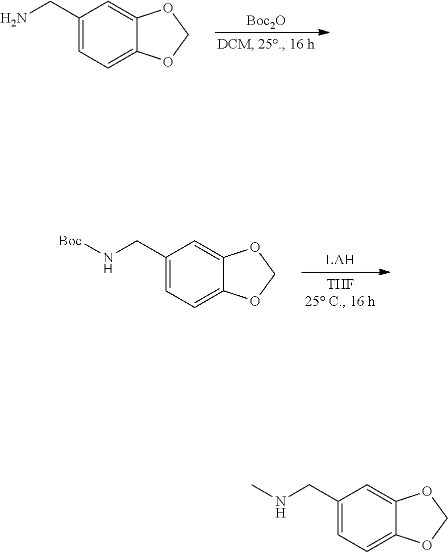 [SMILES: "Boc2O" ; NCc1ccc2c(c1)OCO2 ; CC(C)(C)OC(=O)NCc1ccc2c(c1)OCO2 ; CNCc1ccc2c(c1)OCO2]

To a solution of 1,3-benzodioxol-5-ylmethanamine (1 g, 6.62 mmol, 826.45 uL, 1 eq.) in DCM (10 mL) was added Boc2O (1.59 g, 7.28 mmol, 1.67 mL, 1.1 eq.), then the mixture was stirred at 25° C. for 2 h. LC-MS showed 1,3-benzodioxol-5-ylmethanamine (1 g, 6.62 mmol, 826.45 uL, 1 eq.) was consumed completely and one main peak with desired mass was detected. The reaction mixture was concentrated under reduced pressure to get crude product. The residue was diluted with H2O and extracted with Ethyl acetate 90 mL. The combined organic layers were dried over Na2SO4, filtered and concentrated under reduced pressure to give a crude product. The crude product was used to next step without further purification. Tert-butyl N-(1,3-benzodioxol-5-ylmethyl)carbamate (1.5 g, 5.97 mmol, 90.24% yield) was obtained as yellow oil. LCMS: MS: 274.3 [M+H]⁺.

To a mixture of tert-butyl N-(1,3-benzodioxol-5-ylmethyl)carbamate (0.3 g, 1.19 mmol, 1 eq.) in THE (5 mL) was added LAH (135.94 mg, 3.58 mmol, 3 eq.) at 0° C. under N₂, then the mixture was stirred at 25° C. for 16 h. TLC (Petroleum ether:Ethyl acetate=1:1) showed tert-butyl N-(1,3-benzodioxol-5-ylmethyl)carbamate (0.3 g, 1.19 mmol, 1 eq.) was consumed, and one new spot was detected. The mixture was quenched by adding Na₂SO₄.10H₂O (2 g) under N₂ at 0° C., then was filtered and washed with MeOH, concentrated to obtain the product. The product was used to next step directly without further purification. 1-(1,3-Benzodioxol-5-yl)-N-methyl-methanamine (0.15 g, 908.05 umol, 76.06% yield) was obtained as white solid.

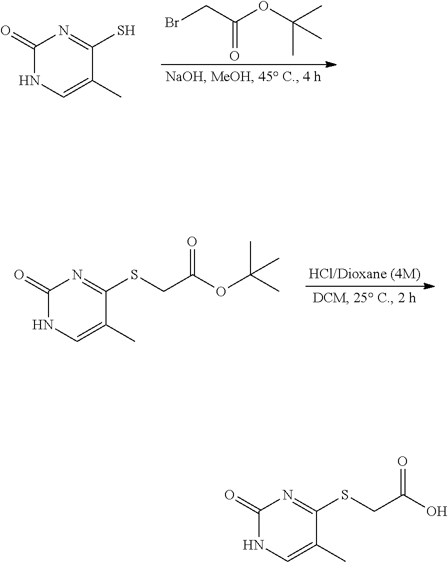

To a solution of 5-methyl-4-sulfanyl-1H-pyrimidin-2-one (960 mg, 6.75 mmol, 1 eq.) in MeOH (10 mL) was added NaOH (540.17 mg, 13.50 mmol, 2 eq.) and tert-butyl 2-bromoacetate (1.32 g, 6.75 mmol, 997.74 uL, 1 eq.), then the mixture was stirred at 45° C. for 4 h. LC-MS showed 5-methyl-4-sulfanyl-1H-pyrimidin-2-one (960 mg, 6.75 mmol, 1 eq.) was consumed completely and one main peak with desired mass was detected. The reaction mixture was concentrated under reduced pressure to remove MeOH. The residue was diluted with H₂O and extracted with Ethyl acetate. The combined organic layers were dried over Na₂SO₄, filtered and concentrated under reduced pressure to give a crude product. The crude product was triturated by Petroleum ether:Ethyl acetate=1:5 at 25° C. Tert-butyl 2-[(5-methyl-2-oxo-1H-pyrimidin-4-yl)sulfanyl]acetate (0.75 g, 2.93 mmol, 43.34% yield) was obtained as a yellow solid. LCMS: MS: 201.0 [M+H]⁺.

To a solution of tert-butyl 2-[(5-methyl-2-oxo-1H-pyrimidin-4-yl)sulfanyl]acetate (0.5 g, 1.95 mmol, 1 eq.) in DCM (4 mL) was added HCl/dioxane (4 M, 4 mL, 8.20 eq.), then the mixture was stirred at 25° C. for 2 h. LC-MS showed tert-butyl 2-[(5-methyl-2-oxo-1H-pyrimidin-4-yl)sulfanyl]acetate (0.5 g, 1.95 mmol, 1 eq.) was consumed completely and one main peak with desired mass was detected. The reaction mixture was concentrated under reduced pressure to get crude product. 2-[(5-Methyl-2-oxo-1H-pyrimidin-4-yl)sulfanyl]acetic acid (0.39 g, 1.95 mmol, 99.86% yield) was obtained as a white solid. LCMS: MS: 201.1 [M+H]⁺.

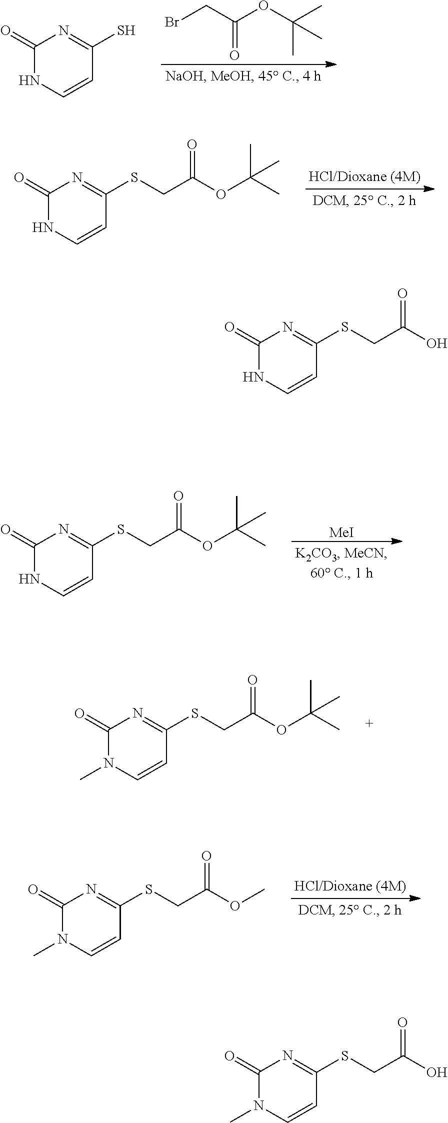

To a mixture of tert-butyl 2-[(2-oxo-1H-pyrimidin-4-yl)sulfanyl]acetate (0.2 g, 825.44 umol, 1 eq.) in DMF (2 mL) was added K₂CO₃ (228.16 mg, 1.65 mmol, 2 eq.) and Mel (128.88 mg, 907.99 umol, 56.53 uL, 1.1 eq.), then the mixture was stirred at 25° C. for 2 h. TLC (Ethyl acetate:Petroleum ether=10:1, R_{f}=0.6) showed tert-butyl 2-[(2-oxo-1H-pyrimidin-4-yl)sulfanyl]acetate (0.2 g, 825.44 umol, 1 eq.) was consumed, and two new spots were detected. The mixture was quenched by addition to NH₄Cl solution at 0° C., then extracted with Ethyl acetate. The organic phase was separated and washed with saturated brine. After that the organic phase was dried over Na₂SO₄, filtered and concentrated under reduced pressure to give a crude product. The crude product was purified by prep-TLC (Ethyl acetate:Petroleum ether=10:1, R_{f}=0.6). Tert-butyl 2-(1-methyl-2-oxo-pyrimidin-4-yl)sulfanylacetate (0.1 g, 390.14 umol, 47.26% yield) was obtained as yellow solid and confirmed by special NMR. Methyl 2-(1-methyl-2-oxo-pyrimidin-4-yl)sulfanylacetate (0.05 g, 233.38 umol, 28.27% yield) was obtained as yellow solid and confirmed by special NMR.

To a solution of tert-butyl 2-(1-methyl-2-oxo-pyrimidin-4-yl)sulfanylacetate (0.12 g, 468.16 umol, 1 eq.) in DCM (2 mL) was added HCl/dioxane (4 M, 2 mL, 17.09 eq.). The mixture was stirred at 25° C. for 16 h. LC-MS showed tert-butyl 2-(1-methyl-2-oxo-pyrimidin-4-yl)sulfanylacetate (0.12 g, 468.16 umol, 1 eq.) was consumed completely and one main peak with desired mass was detected. The reaction mixture was concentrated under reduced pressure to get crude product. 2-(1-Methyl-2-oxo-pyrimidin-4-yl)sulfanylacetic acid (0.0937 g, 468.00 umol, 99.96% yield) was obtained as a white solid. LCMS: MS: 223.3 [M+H]⁺.

### Synthesis of Compound I-3

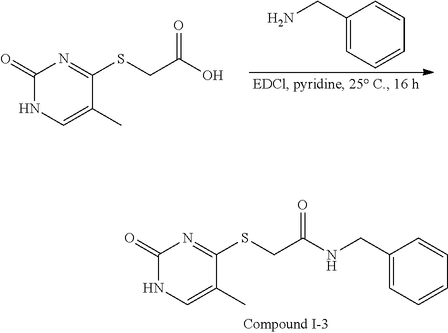 [SMILES: Cc1c[nH]c(=O)nc1SCC(=O)O ; NCc1ccccc1 ; Cc1c[nH]c(=O)nc1SCC(=O)NCc1ccccc1]

To a solution of 2-[(5-methyl-2-oxo-1H-pyrimidin-4-yl)sulfanyl]acetic acid (0.06 g, 299.68 umol, 1 eq.) in pyridine (2 mL) was added EDCI (86.17 mg, 449.52 umol, 1.5 eq.) and phenylmethanamine (32.11 mg, 299.68 umol, 32.67 uL, 1 eq.), then the mixture was stirred at 25° C. for 16 h. LC-MS showed 2-[(5-methyl-2-oxo-1H-pyrimidin-4-yl)sulfanyl]acetic acid (0.06 g, 299.68 umol, 1 eq.) was consumed completely and one main peak with desired mass was detected. The crude reaction mixture was concentrated under reduced pressure to obtain the crude product. The crude product was purified by prep-HPLC (column: Waters xbridge 150*25 mm 10 um; mobile phase: [water(NH₄HCO₃)-MeCN]; B %: 7%-37%, 8 min). N-Benzyl-2-[(5-methyl-2-oxo-1H-pyrimidin-4-yl)sulfanyl]acetamide (0.011 g, 37.64 umol, 12.56% yield, 99% purity) was obtained as a white solid which was confirmed by LCMS and ¹HNMR. LCMS: MS: 290.1 [M+H]⁺. ¹H NMR (400 MHz, DMSO-d6): δ 11.41 (br s, 1H), 8.69 (br t, J=5.8 Hz, 1H), 7.53 (s, 1H), 7.35-7.19 (m, 5H), 4.30 (d, J=6.0 Hz, 2H), 3.96 (s, 2H), 1.96 (s, 3H).

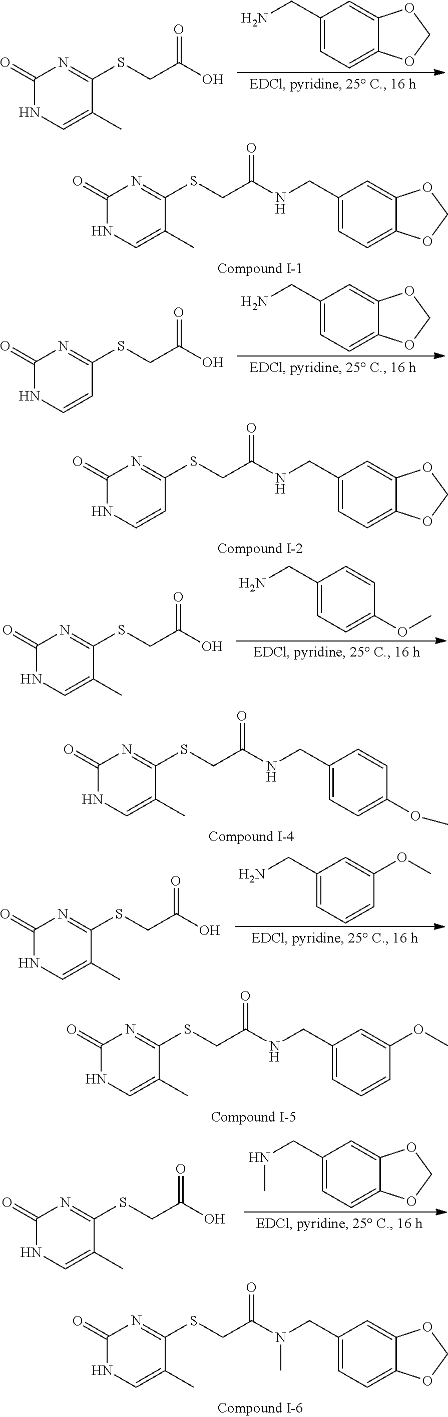

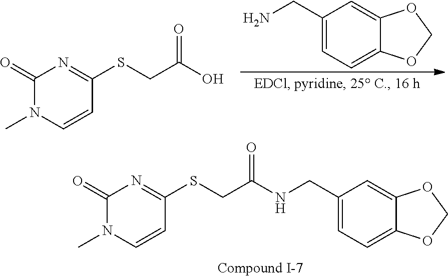 [SMILES: Cn1ccc(SCC(=O)O)nc1=O ; NCc1ccc2c(c1)OCO2 ; Cn1ccc(SCC(=O)NCc2ccc3c(c2)OCO3)nc1=O]

Compounds I-1, I-2, I-4, I-5, I-6 and I-7 were synthesized similarly according to the schemes shown above.

### Example 2: General Synthesis of Compounds I-165, I-166 and I-167

Compound I-165 was synthesized according to the scheme shown below. Chiral separation of compound I-165 with supercritical fluid chromatography afforded compounds I-166 and I-167.

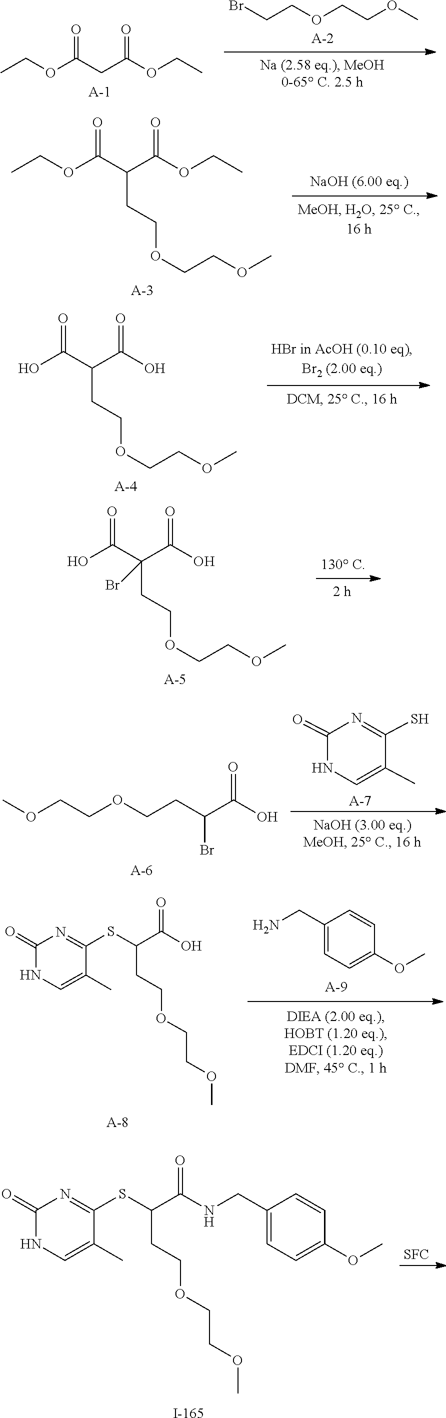

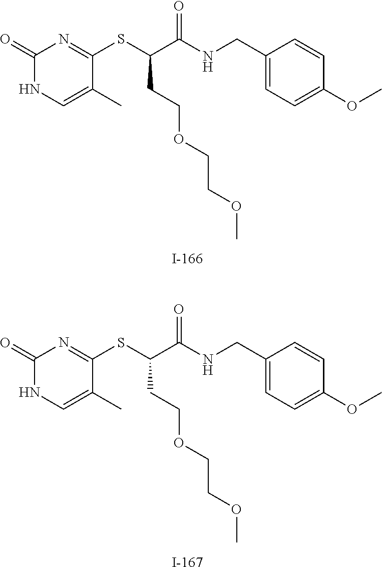 [SMILES: COCCOCC[C@@H](Sc1nc(=O)[nH]cc1C)C(=O)NCc1ccc(OC)cc1 ; "I-166" ; COCCOCC[C@H](Sc1nc(=O)[nH]cc1C)C(=O)NCc1ccc(OC)cc1 ; "I-167"]

### Example 3: General Synthesis of Compounds I-171, I-172 and I-173

Compound I-171 was synthesized according to the scheme shown below. Chiral separation of compound I-171 with supercritical fluid chromatography afforded compounds I-172 and I-173.

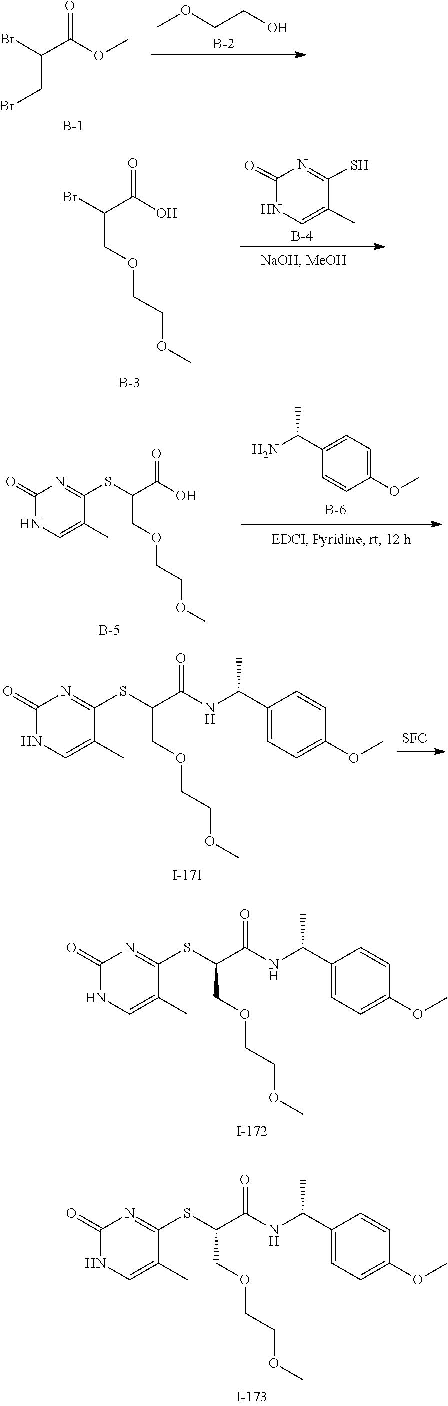

### Example 4: General Synthesis of Compounds I-258 and I-260

Compounds I-258 and I-260 were synthesized according to the scheme shown below.

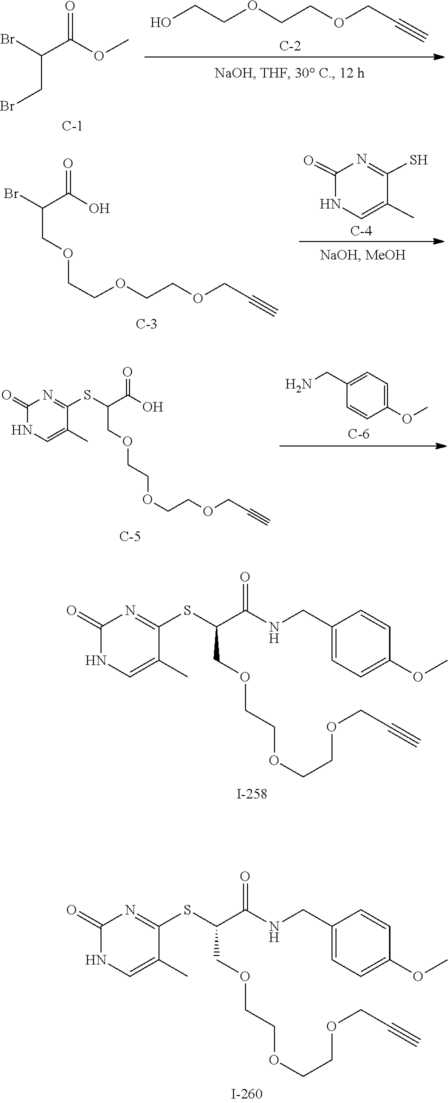

### Example 5: General Synthesis of Compounds I-349, I-351, I-367, I-368, I-329, I-371, I-397 and I-398

Compounds I-349, I-351, I-367, I-368, I-329, I-371, I-397 and 1-398 were synthesized according to the schemes shown below.

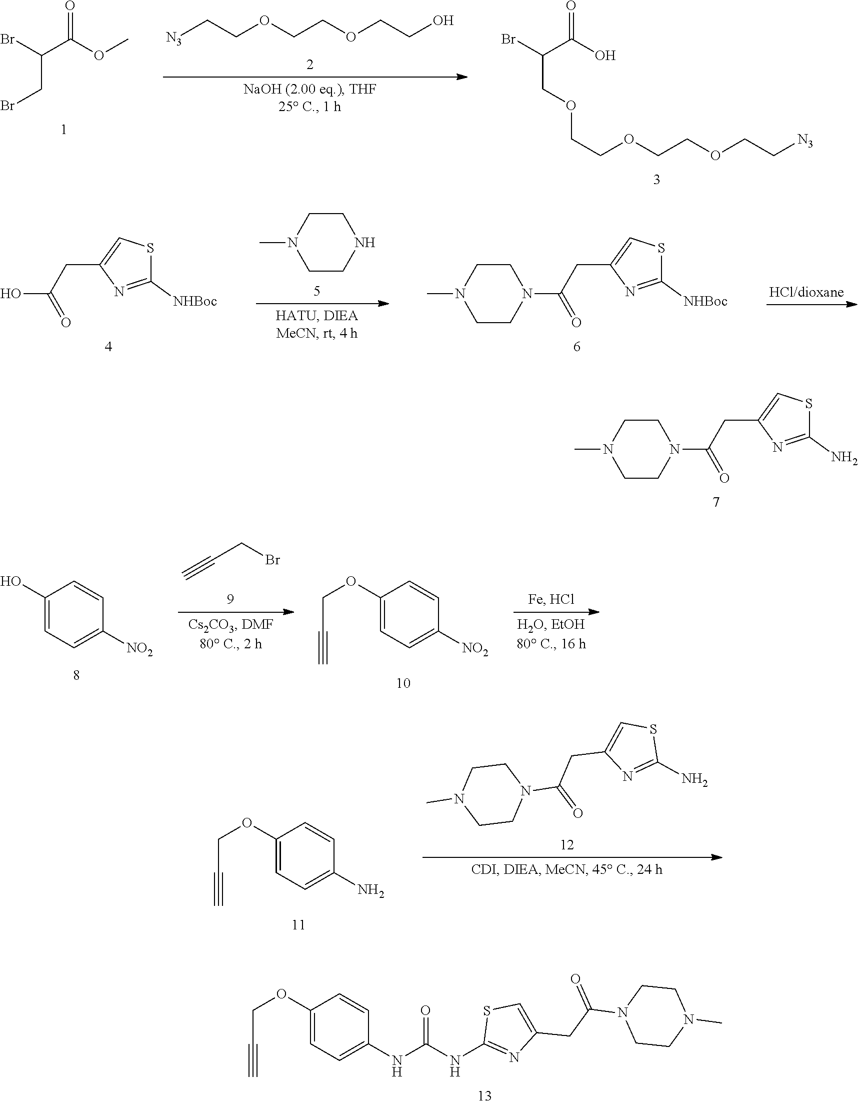

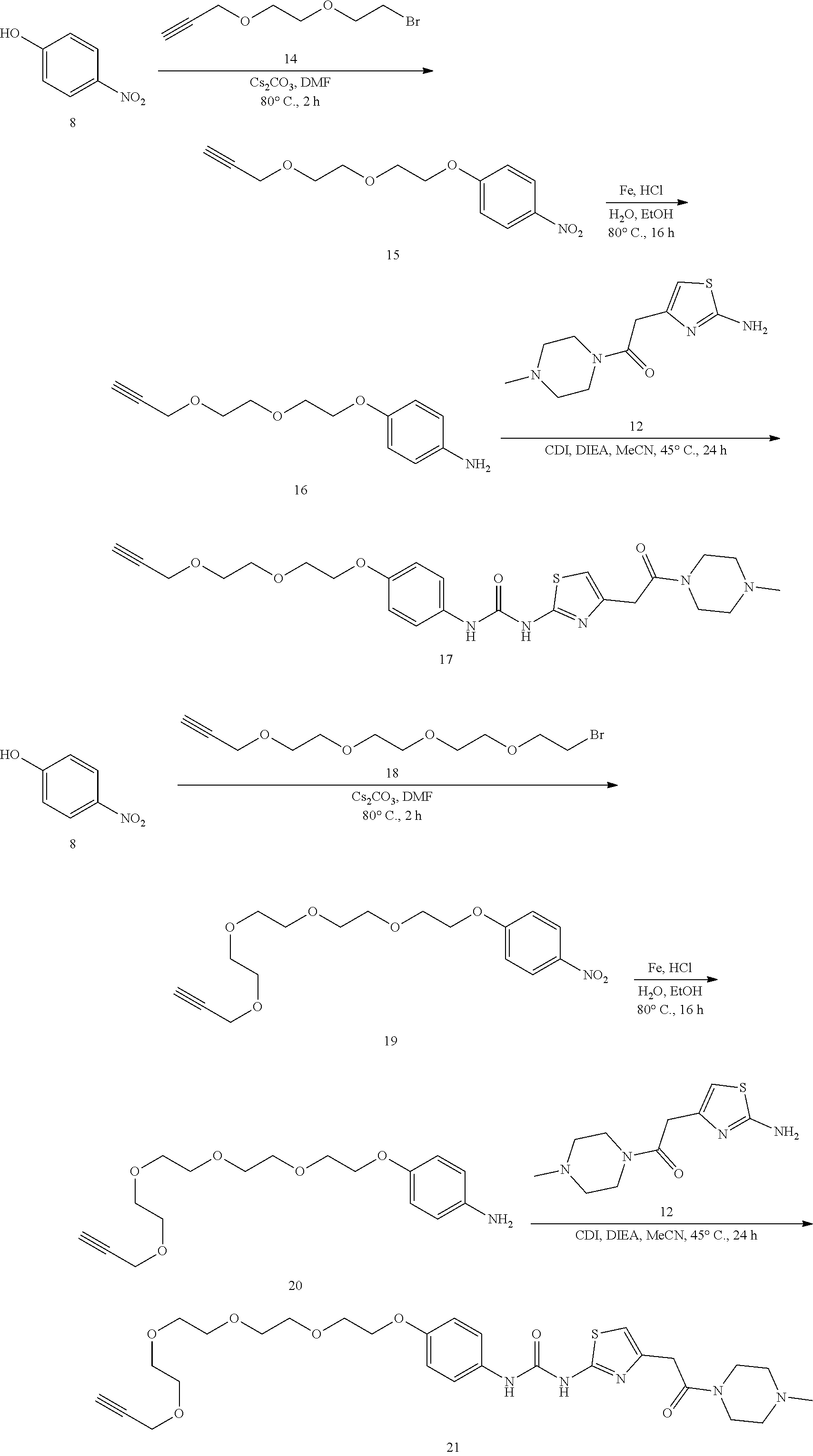

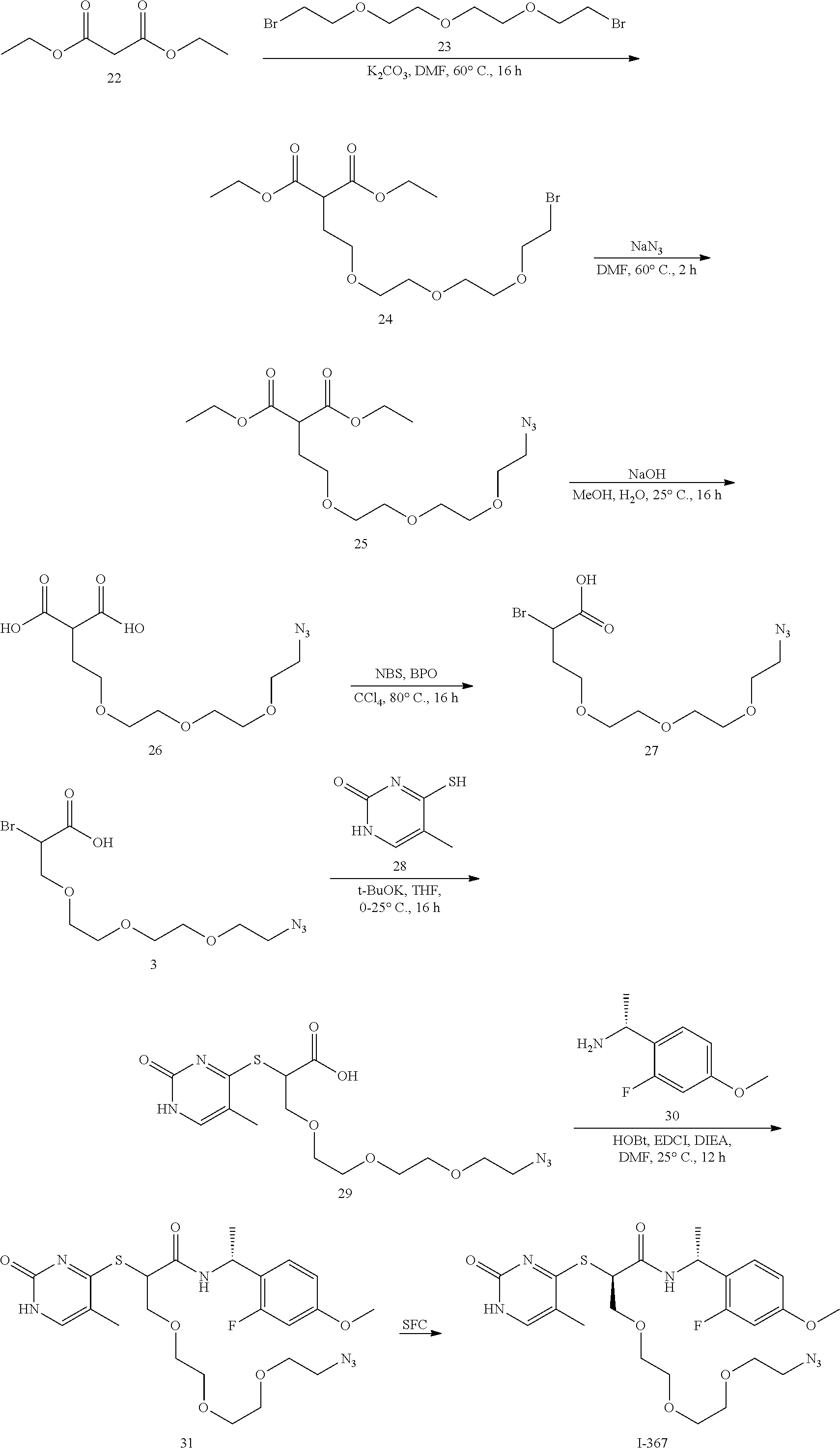

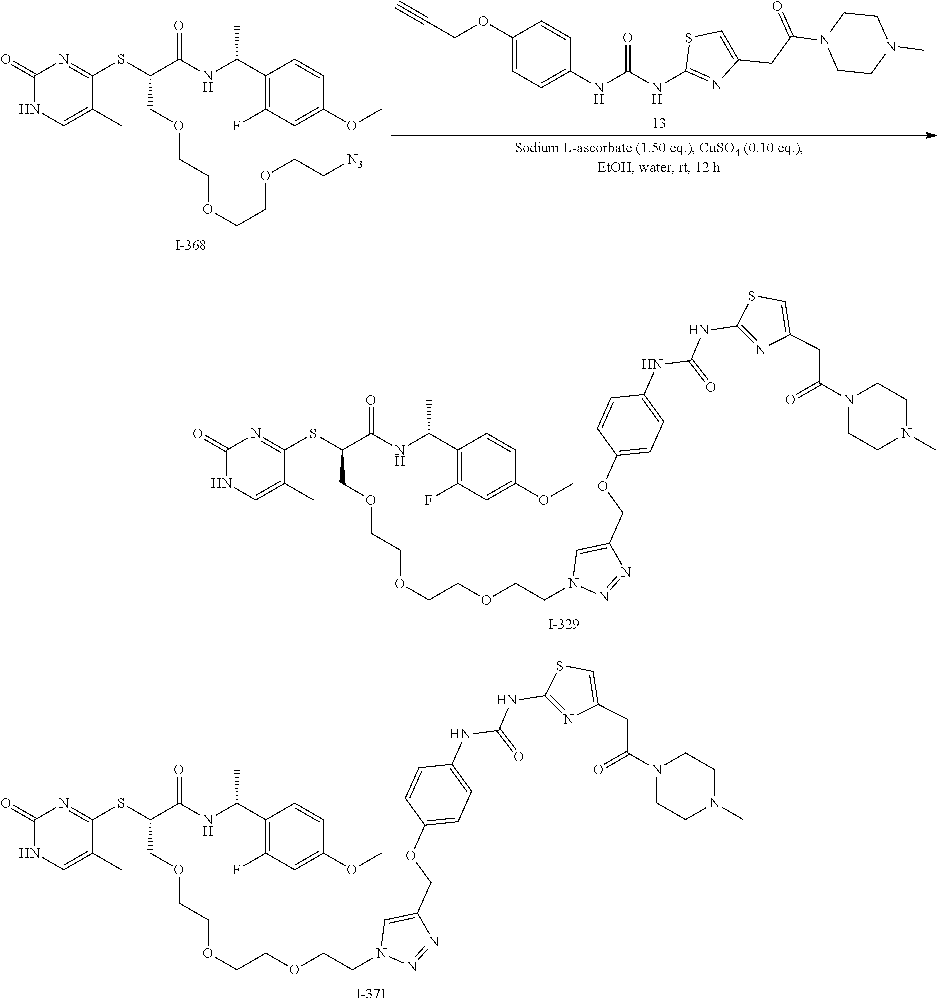 [SMILES: COc1ccc([C@@H](C)NC(=O)[C@H](COCCOCCOCCN=[N+]=[N-])Sc2nc(=O)[nH]cc2C)c(F)c1 ; C#CCOc1ccc(NC(=O)Nc2nc(CC(=O)N3CCN(C)CC3)cs2)cc1 ; COc1ccc([C@@H](C)NC(=O)[C@@H](COCCOCCOCCn2cc(COc3ccc(NC(=O)Nc4nc(CC(=O)N5CCN(C)CC5)cs4)cc3)nn2)Sc2nc(=O)[nH]cc2C)c(F)c1 ; COc1ccc([C@@H](C)NC(=O)[C@H](COCCOCCOCCn2cc(COc3ccc(NC(=O)Nc4nc(CC(=O)N5CCN(C)CC5)cs4)cc3)nn2)Sc2nc(=O)[nH]cc2C)c(F)c1]

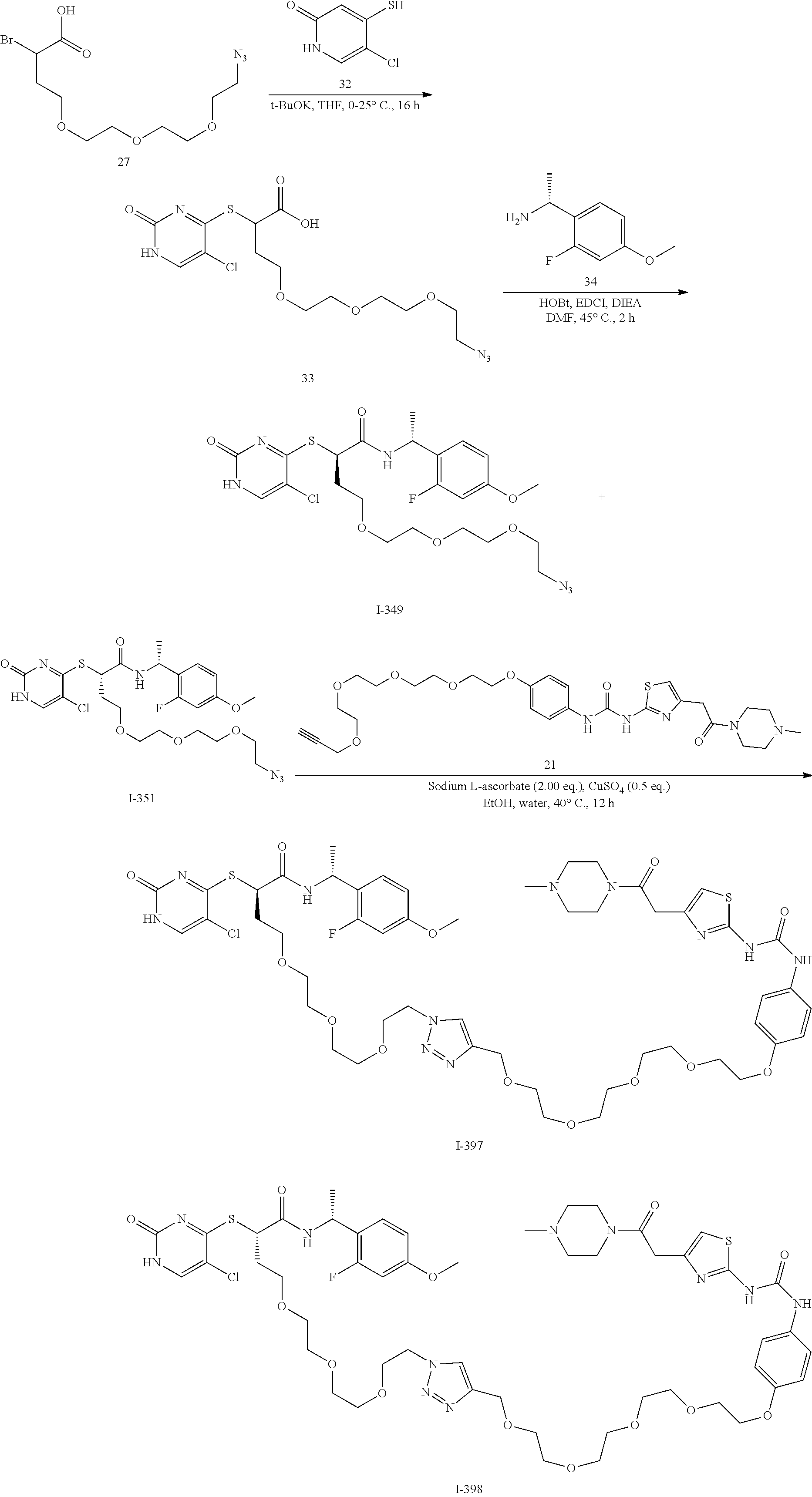

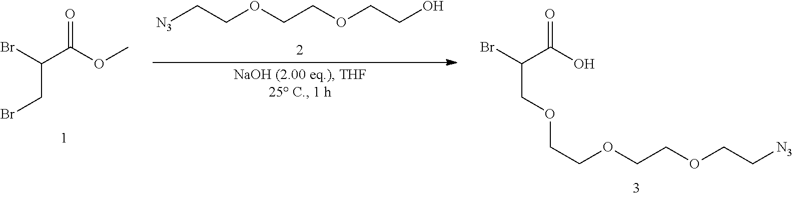

To a solution of methyl 2,3-dibromopropanoate (14.04 g, 57.08 mmol, 7.22 mL, 1.00 eq.) and NaOH (4.57 g, 114.16 mmol, 2.00 eq.) in THF (20 mL) was added slowly 2-[2-(2-azidoethoxy)ethoxy]ethanol (10.0 g, 57.08 mmol, 1.00 eq.). The mixture was stirred at 25° C. for 1 h. The reaction mixture was concentrated under reduced pressure to remove THF (20 mL). Without further purification, compound 3-[2-[2-(2-azidoethoxy)ethoxy]ethoxy]-2-bromo-propanoic acid (10.0 g, 30.66 mmol, 53.71% yield) was obtained as a white solid. LCMS: 349.0, 350.3 [M+Na]⁺.

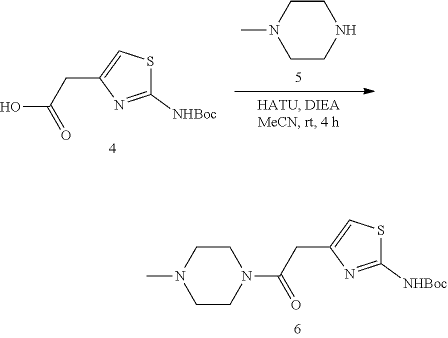

A mixture of 2-[2-(tert-butoxycarbonylamino)thiazol-4-yl]acetic acid (50 g, 193.58 mmol, 1.00 eq.), 1-methylpiperazine (23.27 g, 232.29 mmol, 25.77 mL, 1.20 eq.), HATU (95.69 g, 251.65 mmol, 1.30 eq.), DIEA (50.04 g, 387.16 mmol, 67.44 mL, 2.00 eq.) in MeCN (500 mL) was stirred at 25° C. for 4 h. LC-MS showed 2-[2-(tert-butoxycarbonylamino)thiazol-4-yl]acetic acid (50 g, 193.58 mmol, 1.00 eq.) was consumed completely and one main peak with desired mass was detected. The mixture was filtered and the filter cake washed with MeCN 40 mL (20 mL*2) to get a crude product. Without further purification, compound tert-butyl N-[4-[2-(4-methylpiperazin-1-yl)-2-oxo-ethyl]thiazol-2-yl]carbamate (32.8 g, 96.35 mmol, 49.77% yield) was obtained as a white solid. LCMS: 341.0 [M+H]⁺.

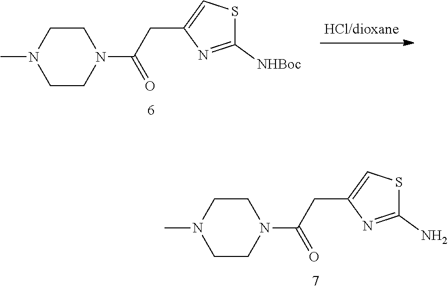 [SMILES: CN1CCN(C(=O)Cc2csc(NC(=O)OC(C)(C)C)n2)CC1 ; CN1CCN(C(=O)Cc2csc(N)n2)CC1]

To a solution of tert-butyl N-[4-[2-(4-methylpiperazin-1-yl)-2-oxo-ethyl]thiazol-2-yl]carbamate (32.8 g, 96.35 mmol, 1.00 eq.) in HCl/dioxane (2 M, 250 mL, 5.19 eq.) was stirred at 45° C. for 24 h. LC-MS showed tert-butyl N-[4-[2-(4-methylpiperazin-1-yl)-2-oxo-ethyl]thiazol-2-yl]carbamate (32.8 g, 96.35 mmol, 1 eq.) was consumed completely and one main peak with desired mass was detected. The reaction mixture was concentrated under reduced pressure to get crude product. Without further purification, compound 2-(2-aminothiazol-4-yl)-1-(4-methylpiperazin-1-yl)ethanone (33 g, 95.38 mmol, 99.00% yield, 80% purity, HCl) was obtained as a white solid. LCMS: 503.2 [2M+Na]⁺.

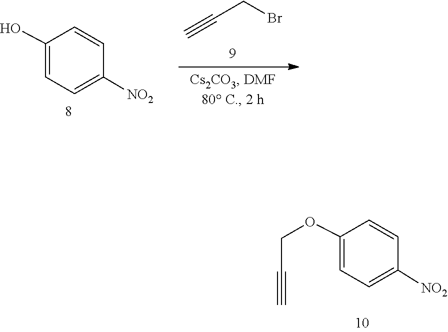

A mixture of 4-nitrophenol (20 g, 143.77 mmol, 1 eq.), 3-bromoprop-1-yne (32.07 g, 215.66 mmol, 23.24 mL, 1.5 eq.), Cs₂CO₃ (93.69 g, 287.55 mmol, 2 eq.) in DMF (300 mL) was stirred at 80° C. for 2 h. TLC (Petroleum ether:Ethyl acetate=5:1) indicated 4-nitrophenol (20 g, 143.77 mmol, 1 eq.) was consumed completely and one new spot (R_{f}=0.80) formed. The reaction was clean according to TLC. The reaction mixture was quenched by addition H₂O 3000 mL at 25° C., and then extracted with Ethyl acetate 6000 mL (2000 mL*3), the combined organic layers were washed with saturated sodium chloride solution 4000 mL (2000 mL*2), dried over Na₂SO₄, filtered and concentrated under reduced pressure to give a crude product. Without further purification, compound 1-nitro-4-prop-2-ynoxy-benzene (25.5 g, 133.67 mmol, 92.97% yield, 92.862% purity) was obtained as a brown solid.

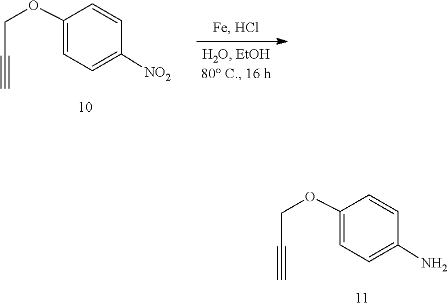

To a solution of 1-nitro-4-prop-2-ynoxy-benzene (10 g, 52.88 mmol, 1.00 eq.) in H₂O (50 mL) and EtOH (250 mL) was added HCl (12 M, 8.81 mL, 2.00 eq) and Fe (14.76 g, 264.38 mmol, 5.00 eq), then the mixture was stirred at 80° C. for 16 h. TLC (Petroleum ether:Ethyl acetate=3:1, R_{f}=0.2) indicated 1-nitro-4-prop-2-ynoxy-benzene (10 g, 52.88 mmol, 1 eq.) was consumed completely and one new spot formed. The reaction was clean according to TLC. The mixture was filtered through diatomaceous earth and washed with methanol 900 mL (300 mL*3), the filtrate was concentrated under reduced pressure to get crude product. The residue was purified by column chromatography (SiO₂, Petroleum ether:Ethyl acetate=20:1 to 8:1). Compound 4-prop-2-ynoxyaniline (3.2 g, 21.74 mmol, 41.12% yield) was obtained as a yellow oil which was confirmed by ¹H NMR. ¹H NMR: (400 MHz, DMSO-d6): δ=6.75-6.66 (m, 2H), 6.56-6.47 (m, 2H), 4.67 (s, 2H), 4.60 (d, J=2.3 Hz, 2H), 3.47 (t, J=2.3 Hz, 1H).

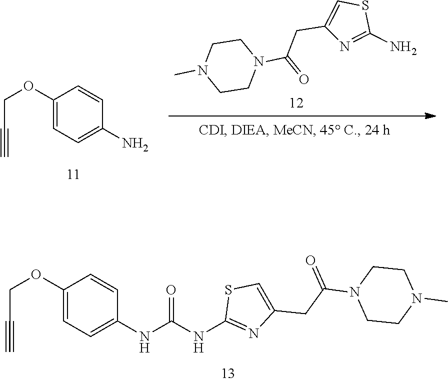

To a solution of 2-(2-aminothiazol-4-yl)-1-(4-methylpiperazin-1-yl)ethanone (0.5 g, 2.08 mmol, 1.00 eq.) in MeCN (20 mL) was added CDI (404.82 mg, 2.50 mmol, 1.20 eq.) and DIEA (537.79 mg, 4.16 mmol, 724.78 μL, 2.00 eq.) at 25° C., after addition, the mixture was stirred at 45° C. for 16 h, and then 4-prop-2-ynoxyaniline (306.20 mg, 2.08 mmol, 1.00 eq.) was added at 25° C., the resulting mixture was stirred at 45° C. for 8 h. LC-MS showed 2-(2-aminothiazol-4-yl)-1-(4-methylpiperazin-1-yl)ethanone (0.5 g, 2.08 mmol, 1.00 eq.) was consumed completely and one main peak with desired mass was detected. The reaction mixture was quenched by addition MeOH 20 mL at 25° C., and then the mixture was concentrated under reduced pressure to get a crude product. The crude product was purified by prep-HPLC (column: Phenomenex luna C18 (250*70 mm, 10 um); mobile phase: [water(NH₄HCO₃)-ACN]; gradient: 20%-50% B over 20 min). Compound 1-[4-[2-(4-methylpiperazin-1-yl)-2-oxo-ethyl]thiazol-2-yl]-3-(4-prop-2-ynoxyphenyl)urea (0.5 g, 1.21 mmol, 29.06% yield) was obtained as a white solid. LCMS: 414.1 [M+H]⁺.

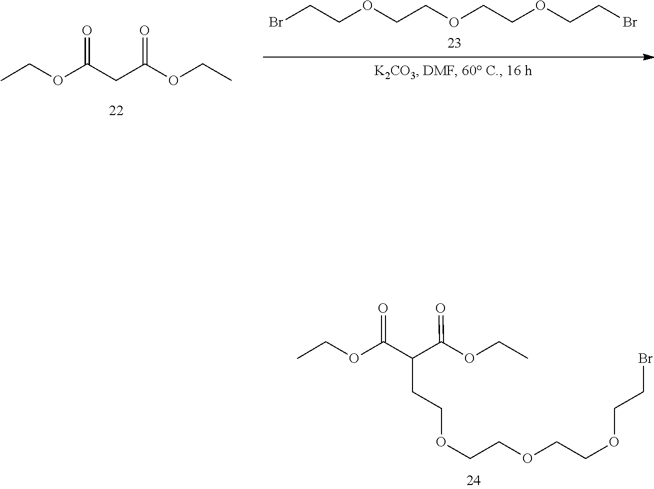 [SMILES: BrCCOCCOCCOCCBr ; CCOC(=O)CC(=O)OCC ; CCOC(=O)C(CCOCCOCCOCCBr)C(=O)OCC]

To a solution of diethyl propanedioate (10.43 g, 65.10 mmol, 9.88 mL, 1.00 eq.) in DMF (200 mL) was added K₂CO₃ (22.49 g, 162.75 mmol, 2.50 eq.) and 1-(2-bromoethoxy)-2-[2-(2-bromoethoxy)ethoxy]ethane (25 g, 78.12 mmol, 1.20 eq.), then the mixture was stirred at 60° C. for 16 h. TLC (Petroleum ether:Ethyl acetate=3:1) indicated 20% of diethyl propanedioate (10.43 g, 65.10 mmol, 9.88 mL, 1.00 eq.) was remained, and one major new spot (R_{f}=0.35) with larger polarity was detected. The reaction mixture was diluted with H₂O 1000 mL and extracted with Ethyl acetate 1500 mL (300 mL*5), the combined organic layers were washed with H₂O 600 mL (200 mL*3), dried over Na₂SO₄, filtered and concentrated under reduced pressure to give a crude product. The crude product was purified by MPLC (SiO₂, Petroleum ether/Ethyl acetate=50/1 to 10/1). Compound diethyl 2-[2-[2-[2-(2-bromoethoxy)ethoxy]ethoxy]ethyl]propanedioate (10.8 g, 27.05 mmol, 41.55% yield) was obtained as a yellow oil.

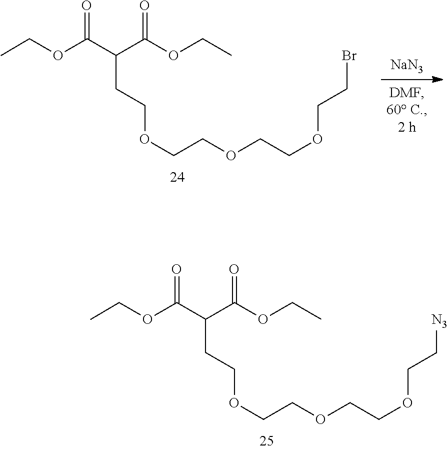 [SMILES: CCOC(=O)C(CCOCCOCCOCCBr)C(=O)OCC ; CCOC(=O)C(CCOCCOCCOCCN=[N+]=[N-])C(=O)OCC ; "NaN3"]

To a solution of diethyl 2-[2-[2-[2-(2-bromoethoxy)ethoxy]ethoxy]ethyl]propanedioate (5.0 g, 12.52 mmol, 1.00 eq.) in DMF (30 mL) was added NaN₃ (1 g, 15.38 mmol, 1.23 eq), then the mixture was stirred at 60° C. for 2 h. TLC (Petroleum ether:Ethyl acetate=2:1, R_{f}=0.45) indicated diethyl 2-[2-[2-[2-(2-bromoethoxy)ethoxy]ethoxy]ethyl]propanedioate (5.0 g, 12.52 mmol, 1.00 eq.) was consumed completely and one new spot formed. The reaction mixture was quenched by addition H₂O 300 mL at 25° C., and then extracted with Ethyl acetate 450 mL (150 mL*3). The combined organic layers were washed with H₂O 200 mL (100 mL*2), dried over Na₂SO₄, filtered and concentrated under reduced pressure to give a crude product. Without further purification. Compound diethyl 2-[2-[2-[2-(2-azidoethoxy)ethoxy]ethoxy]ethyl]propanedioate (3.92 g, 10.85 mmol, 86.62% yield) was obtained as a yellow oil.

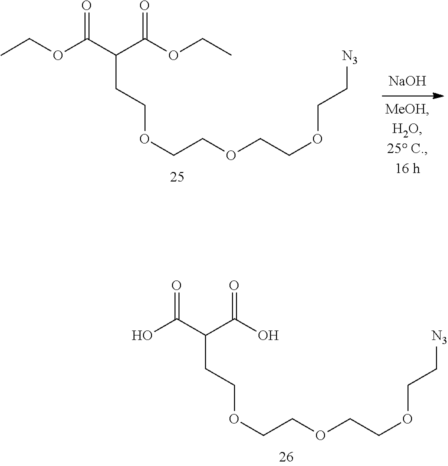

To a solution of diethyl 2-[2-[2-[2-(2-azidoethoxy)ethoxy]ethoxy]ethyl]propanedioate (9.5 g, 26.29 mmol, 1.00 eq) in MeOH (100 mL) and H₂O (10 mL) was added NaOH (4.21 g, 105.15 mmol, 4.00 eq), then the mixture was stirred at 25° C. for 16 h. LC- showed one main peak with desired mass. TLC (Petroleum ether:Ethyl acetate=1:1) indicated diethyl 2-[2-[2-[2-(2-azidoethoxy)ethoxy]ethoxy]ethyl]propanedioate (9.5 g, 26.29 mmol, 1 eq) was consumed completely and one new spot (Rf=0.01) formed. The reaction mixture was concentrated under reduced pressure to remove MeOH, the residue was diluted with H₂O (10 mL) and cooled to 0° C., and then 1N HCl was slowly added until pH=8, then the reaction mixture was concentrated under reduced pressure to get a crude product. Without further purification, compound 2-[2-[2-[2-(2-azidoethoxy)ethoxy]ethoxy]ethyl]propanedioic acid (13 g, 25.55 mmol, 97.19% yield, 60% purity) was obtained as a white solid. LCMS: 304.1 [M−H]⁻.

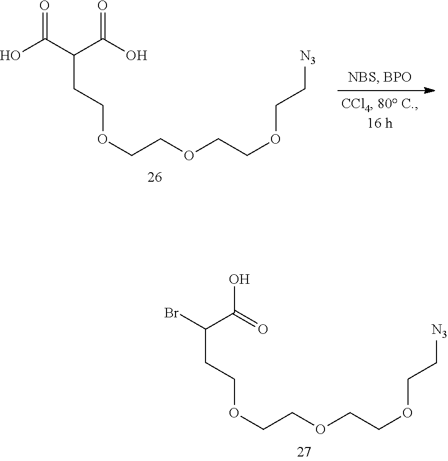

A mixture of 2-[2-[2-[2-(2-azidoethoxy)ethoxy]ethoxy]ethyl]propanedioic acid (6 g, 11.79 mmol, 60% purity. 1.00 eq.), BPO (285.64 mg, 1.18 mmol, 0.10 eq.), NBS (4.20 g, 23.58 mmol, 2.00 eq.) in CCl₄ (100 mL) was stirred at 80° C. for 16 h. LC-MS showed 2-[2-[2-[2-(2-azidoethoxy)ethoxy]ethoxy]ethyl]propanedioic acid (6 g, 11.79 mmol, 60% purity, 1 eq) was consumed completely and one peak with desired mass was detected. The reaction mixture was concentrated under reduced pressure to get crude product. Without further purification, compound 4-[2-[2-(2-azidoethoxy)ethoxy]ethoxy]-2-bromo-butanoic acid (11 g, 4.85 mmol, 41.13% yield, 15% purity) was obtained as a yellow solid. LCMS: 338.0 [M−H]⁻.

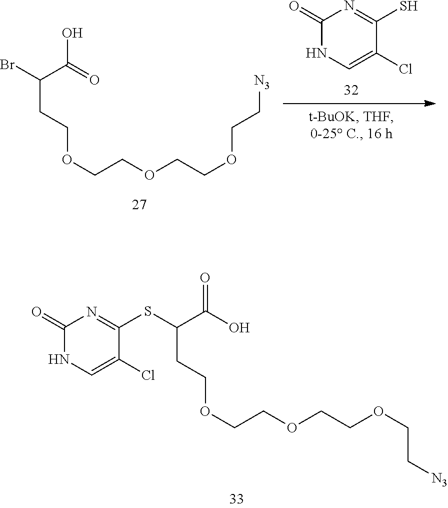

To a solution of 5-chloro-4-sulfanyl-1H-pyrimidin-2-one (501.89 mg, 3.09 mmol, 15% purity, 1.00 eq.) in THF (30 mL) was added dropwise tBuOK (692.73 mg, 6.17 mmol, 2.00 eq.) at 0° C., after addition, the mixture was stirred at 0° C. for 2 h, and then 4-[2-[2-(2-azidoethoxy)ethoxy]ethoxy]-2-bromo-butanoic acid (7 g, 3.09 mmol, 1 eq.) in THF (30 mL) was added dropwise at 0° C., then the resulting mixture was stirred at 25° C. for 14 h. LC-MS showed one peak with desired mass was detected. The mixture was cooled to 0° C. and 1N HCl was slowly added until pH=8, then the reaction mixture was concentrated under reduced pressure to get a crude product. The residue was purified by prep-HPLC (column: Phenomenex luna C18 (250*70 mm, 10 um); mobile phase: [water(NH₄HCO₃)-ACN]; gradient: 1%-25% B over 20 min). Compound 4-[2-[2-(2-azidoethoxy)ethoxy]ethoxy]-2-[(5-chloro-2-oxo-1H-pyrimidin-4-yl)sulfanyl]butanoic acid (0.28 g, 663.73 μmol, 21.50% yield) was obtained as a yellow oil. LCMS: 422.1 [M+H]⁺.

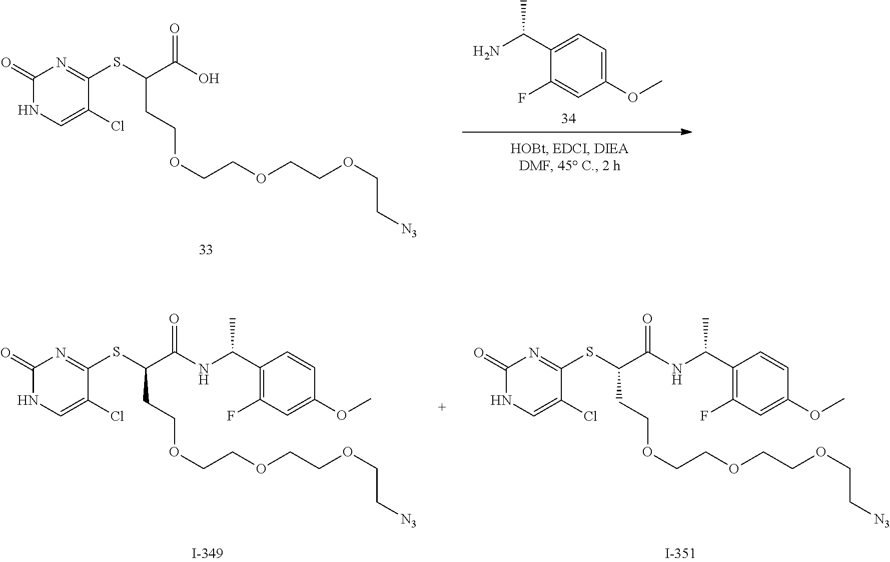

A mixture of 4-[2-[2-(2-azidoethoxy)ethoxy]ethoxy]-2-[(5-chloro-2-oxo-1H-pyrimidin-4-yl)sulfanyl]butanoic acid (0.26 g, 616.32 μmol, 1.00 eq.), (1R)-1-(2-fluoro-4-methoxy-phenyl)ethanamine (125.14 mg, 739.59 μmol, 1.20 eq.), HOBt (99.94 mg, 739.59 μmol, 1.20 eq.), EDCI (141.78 mg, 739.59 μmol, 1.20 eq.) and DIEA (159.31 mg, 1.23 mmol, 214.71 L, 2.00 eq.) in DMF (5 mL) was stirred at 45° C. for 2 h. LC-MS showed 4-[2-[2-(2-azidoethoxy)ethoxy]ethoxy]-2-[(5-chloro-2-oxo-1H-pyrimidin-4-yl)sulfanyl]butanoic acid (0.26 g, 616.32 μmol, 1.00 eq) was consumed completely and two peaks with desired mass were detected. The reaction mixture was quenched by addition H₂O 3 mL at 25° C. The mixture was purified by prep-HPLC (column: Waters Xbridge BEH C18 150*25 mm*5 um; mobile phase: [water(NH₄HCO₃)-ACN]; gradient: 30%-50% B over 10 min). Compound (2R)-4-[2-[2-(2-azidoethoxy)ethoxy]ethoxy]-2-[(5-chloro-2-oxo-1H-pyrimidin-4-yl)sulfanyl]-N-[(1R)-1-(2-fluoro-4-methoxy-phenyl)ethyl]butanamide (0.028 g, 45.65 μmol, 7.41% yield, 93.419% purity) was obtained as a yellow oil which was confirmed by LC-MS, ¹HNMR and SFC. LCMS: 573.2 [M+H]⁺. ¹H NMR: EC10112-345-P1G106A_A (400 MHz, DMSO-d6): δ=7.86 (s, 1H), 7.25-7.18 (m, 1H), 6.62-6.56 (m, 2H), 5.18 (q, J=7.0 Hz, 1H), 4.76 (t, J=7.7 Hz, 1H), 3.75 (s, 3H), 3.69-3.66 (m, 8H), 3.65-3.56 (m, 4H), 3.40-3.35 (m, 2H), 2.27 (qd, J=6.9, 13.8 Hz, 1H), 2.12-2.02 (m, 1H), 1.47 (d, J=7.0 Hz, 3H).

Compound (2S)-4-[2-[2-(2-azidoethoxy)ethoxy]ethoxy]-2-[(5-chloro-2-oxo-1H-pyrimidin-4-yl)sulfanyl]-N-[(1R)-1-(2-fluoro-4-methoxy-phenyl)ethyl]butanamide (0.031 g, 52.58 μmol, 8.53% yield, 97.190% purity) was obtained as a yellow oil which was confirmed by LC-MS, ¹HNMR and SFC. LCMS: 573.2 [M+H]⁺. ¹H NMR: EC10112-345-P1G107A_B (400 MHz, DMSO-d6): δ=7.86 (s, 1H), 7.39 (t, J=8.8 Hz, 1H), 6.76 (dd, J=2.5, 8.6 Hz, 1H), 6.68 (dd, J=2.5, 12.4 Hz, 1H), 5.21 (q, J=7.0 Hz, 1H), 4.73 (dd, J=6.8, 8.9 Hz, 1H), 3.80 (s, 3H), 3.67-3.65 (m, 2H), 3.63 (s, 3H), 3.61-3.50 (m, 6H), 3.45 (ddd, J=5.6, 7.1, 9.8 Hz, 1H), 3.37 (t, J=4.9 Hz, 2H), 2.19 (tdd, J=5.8, 8.6, 14.2 Hz, 1H), 2.04 (qd, J=6.7, 13.3 Hz, 1H), 1.41 (d, J=7.0 Hz, 3H).

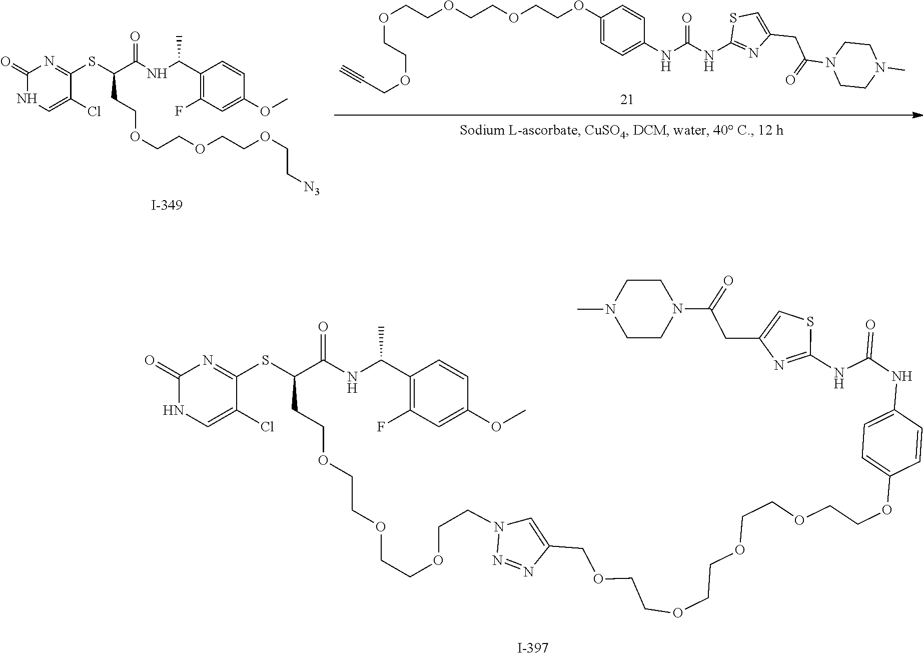 [SMILES: COc1ccc([C@@H](C)NC(=O)[C@@H](CCOCCOCCOCCN=[N+]=[N-])Sc2nc(=O)[nH]cc2Cl)c(F)c1 ; C#CCOCCOCCOCCOCCOc1ccc(NC(=O)Nc2nc(CC(=O)N3CCN(C)CC3)cs2)cc1 ; COc1ccc([C@@H](C)NC(=O)[C@@H](CCOCCOCCOCCn2cc(COCCOCCOCCOCCOc3ccc(NC(=O)Nc4nc(CC(=O)N5CCN(C)CC5)cs4)cc3)nn2)Sc2nc(=O)[nH]cc2Cl)c(F)c1]

To a solution of (2R)-4-[2-[2-(2-azidoethoxy)ethoxy]ethoxy]-2-[(5-chloro-2-oxo-1H-pyrimidin-4-yl)sulfanyl]-N-[(1R)-1-(2-fluoro-4-methoxy-phenyl)ethyl]butanamide (30 mg, 52.35 mol, 1 eq.) and 1-[4-[2-(4-methylpiperazin-1-yl)-2-oxo-ethyl]thiazol-2-yl]-3-[4-[2-[2-[2-(2-prop-2-ynoxyethoxy)ethoxy]ethoxy]ethoxy]phenyl]urea (30.87 mg, 52.35 μmol, 1.00 eq.) in DCM (2 mL) and H₂O (2 mL) was added Sodium L-ascorbate (20.74 mg, 104.71 μmol, 2.00 eq) and CuSO₄.5H₂O (6.54 mg, 26.18 μmol, 0.50 eq). The mixture was stirred at 40° C. for 12 h. LCMS showed (2R)-4-[2-[2-(2-azidoethoxy)ethoxy]ethoxy]-2-[(5-chloro-2-oxo-1H-pyrimidin-4-yl)sulfanyl]-N—[(JR)-1-(2-fluoro-4-methoxyphenyl)ethyl]butanamide (30 mg, 52.35 μmol, 1.00 eq.) was consumed and one main peak with desired mass was detected. The mixture was diluted with H₂O 10 mL and extracted with DCM (10 mL×3). The combined organic layers were concentrated under reduced pressure to give a crude product. The crude product was purified by prep-HPLC (column: Phenomenex Gemini-NX 150*30 mm*5 um; mobile phase: [H₂O (0.2% FA)-ACN]; gradient: 10%-40% B over 20.0 min). Compound (2R)-2-[(5-chloro-2-oxo-1H-pyrimidin-4-yl)sulfanyl]-N-[(1R)-1-(2-fluoro-4-methoxy-phenyl)ethyl]-4-[2-[2-[2-[4-[2-[2-[2-[2-[4-[[4-[2-(4-methylpiperazin-1-yl)-2-oxo-ethyl]thiazol-2-yl]carbamoylamino]phenoxy]ethoxy]ethoxy]ethoxy]ethoxymethyl]triazol-1-yl]ethoxy]ethoxy]ethoxy]butanamide (15 mg, 12.90 μmol, 24.64% yield) was obtained as a white solid. The structure was confirmed by LCMS and ¹HNMR. LCMS: 581.8 [M/2+H]⁺. ¹H NMR: ET22502-1565-p1a1 (400 MHz, DMSO-d6): δ=11.58-10.55 (m, 1H), 9.59-9.30 (m, 1H), 8.79 (br d, J=7.9 Hz, 1H), 8.20 (s, 1H), 8.04 (d, J=3.0 Hz, 2H), 7.42 (br d, J=8.9 Hz, 2H), 7.32 (t, J=8.7 Hz, 1H), 6.89 (br d, J=8.8 Hz, 2H), 6.79-6.59 (m, 3H), 5.02 (br t, J=6.8 Hz, 1H), 4.60-4.47 (m, 5H), 4.08-4.00 (m, 2H), 3.80 (br t, J=5.2 Hz, 2H), 3.71 (s, 5H), 3.67 (s, 2H), 3.57 (br s, 2H), 3.54-3.46 (m, 22H), 2.59 (br s, 2H), 2.44 (br s, 2H), 2.23 (br t, J=4.5 Hz, 3H), 2.16 (s, 3H), 2.07-1.98 (m, 1H), 1.32 (br d, J=6.9 Hz, 3H).

### Example 6: Synthesis of Compounds I-165, I-166 and I-167

Compounds I-165, I-166 and I-167 were synthesized according to the schemes shown below.

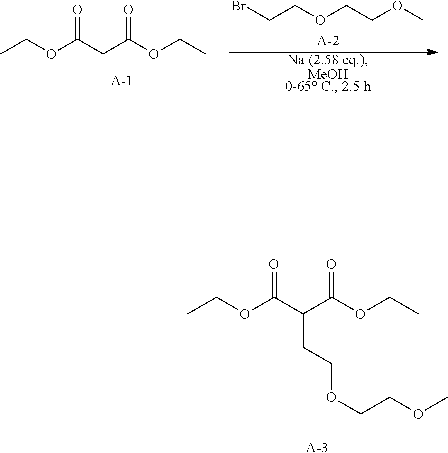

Na (2.59 g, 112 mmol, 2.67 mL, 2.58 eq.) was added to MeOH (50 mL) under N₂ at 0° C., then diethyl propanedioate (7.00 g, 43.7 mmol, 6.64 mL, 1.00 eq.) was dropwise added to the mixture and stirred at 25° C. for 0.5 h, 1-(2-bromoethoxy)-2-methoxy-ethane (8.00 g, 43.7 mmol, 1.00 eq.) was added to the mixture, then the mixture was stirred at 65° C. for 2 h. TLC (Petroleum ether:Ethyl acetate=3:1, R_{f}=0.25) showed diethyl propanedioate was remaining, and one main new spot was detected. The mixture was cooled to 25° C., then was slowly added to H₂O (300 mL), then was extracted with EtOAc 600 mL (200 mL*3). The combined organic layers were washed with brine 300 mL, dried over Na₂SO₄, filtered and concentrated under reduced pressure to give a crude product. The crude product was purified by column chromatography (SiO₂, Petroleum ether:Ethyl acetate=3:1, R_{f}=0.25). Compound diethyl 2-[2-(2-methoxyethoxy)ethyl]propanedioate (6 g, 22.87 mmol, 52.34% yield) was obtained as colorless oil and confirmed by ¹HNMR. ¹HNMR (400 MHz, CHLOROFORM-d): δ 4.21 (dq, J=2.2, 7.1 Hz, 4H), 3.60-3.52 (m, 7H), 3.39 (s, 3H), 2.26-2.16 (m, 2H), 1.28 (t, J=7.1 Hz, 6H).

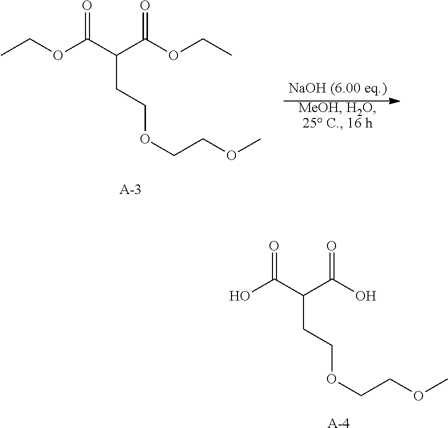

A mixture of diethyl 2-[2-(2-methoxyethoxy)ethyl]propanedioate (4.00 g, 15.2 mmol, 1.00 eq.), NaOH (3.66 g, 91.5 mmol, 6.00 eq.) in MeOH (40.0 mL) and H₂O (5.00 mL) was stirred at 25° C. for 16 h. LCMS showed diethyl 2-[2-(2-methoxyethoxy)ethyl]propanedioate was consumed, and one new peak with desired mass was detected. The mixture was diluted with H₂O 60 mL, then was extracted with DCM 180 mL (60 mL*3). The combined organic layers were washed with brine 30 mL, dried over Na₂SO₄, filtered and concentrated under reduced pressure to give a product. The product was used to next step directly without further purification. Compound 2-[2-(2-methoxyethoxy)ethyl]propanedioic acid (2.4 g, 11.64 mmol, 76.33% yield) was obtained as colorless oil. LCMS: 205.1 [M−H]⁻.

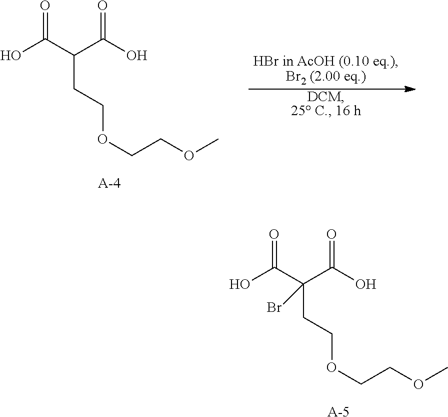

To a mixture of 2-[2-(2-methoxyethoxy)ethyl]propanedioic acid (2.30 g, 11.1 mmol, 1.00 eq.) in DCM (25.0 mL) was added HBr (225 mg, 1.12 mmol, 151 μL, 40% purity, 0.10 eq.) and Br₂ (3.57 g, 22.3 mmol, 1.15 mL, 2.00 eq.), then the mixture was stirred at 25° C. for 16 h. LCMS showed 2-[2-(2-methoxyethoxy)ethyl]propanedioic acid was consumed, and one new peak with desired mass was detected. The mixture was quenched by addition to Na₂SO₃ solution 50 mL at 25° C., then was diluted with H₂O (100 mL), then was extracted with EtOAc 300 mL (100 mL*3). The combined organic layers were washed with brine 50 mL, dried over Na₂SO₄, filtered and concentrated under reduced pressure to give a product. The product was used to next step directly without further purification. Compound 2-bromo-2-[2-(2-methoxyethoxy)ethyl]propanedioic acid (3.00 g, 7.37 mmol, 66.04% yield, 70% purity) was obtained as yellow oil and confirmed by ¹HNMR. LCMS: 283.0, 285.0 [M−H]⁻. ¹H NMR: (400 MHz, CHLOROFORM-d): δ 9.33 (br s, 2H), 3.65 (br t, J=5.9 Hz, 2H), 3.53-3.47 (m, 4H), 3.38-3.26 (m, 3H), 2.63-2.51 (m, 2H).

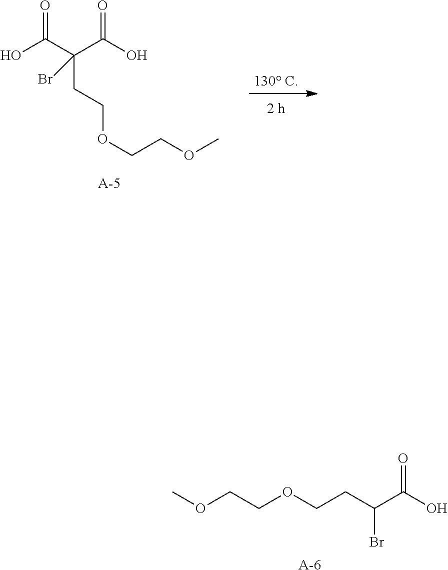

2-bromo-2-[2-(2-methoxyethoxy)ethyl]propanedioic acid (1.50 g, 5.26 mmol, 1.00 eq.) was stirred at 130° C. for 2 h. LCMS showed 2-bromo-2-[2-(2-methoxyethoxy)ethyl]propanedioic acid was consumed, and one new peak with desired mass was detected. No further purification was needed. Compound 2-bromo-4-(2-methoxyethoxy)butanoic acid (0.7 g, 2.90 mmol, 55.19% yield) was obtained as brown oil. LCMS: 239.0, 240.0 [M−H]⁻.

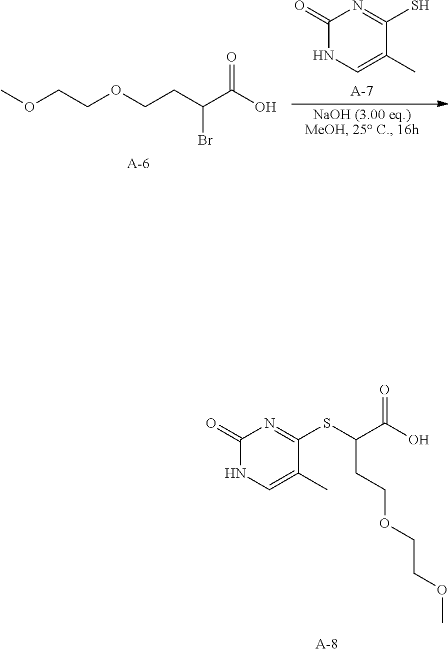

A mixture of 2-bromo-4-(2-methoxyethoxy)butanoic acid (0.300 g, 1.24 mmol, 1.00 eq.), 5-methyl-4-sulfanyl-1H-pyrimidin-2-one (176 mg, 1.24 mmol, 1.00 eq.), NaOH (149 mg, 3.73 mmol, 3.00 eq.) in MeOH (1.00 mL) was stirred at 25° C. for 16 h. LCMS showed 2-bromo-4-(2-methoxyethoxy)butanoic acid was consumed, and one new peak with desired mass was detected. The mixture was concentrated to get a crude product, then H₂O (3 mL) and HCl (1N in water) were added to adjust pH=7 to get the crude product. The crude product was purified by prep-HPLC (column: Phenomenex luna C18 150*25 mm*10 um; mobile phase: [water(HCl)-ACN]; gradient: 4%-34% B over 15 min). Compound 4-(2-methoxyethoxy)-2-[(5-methyl-2-oxo-1H-pyrimidin-4-yl)sulfanyl]butanoic acid (0.07 g, 231.52 μmol, 18.61% yield) was obtained as a yellow solid. LCMS: 303.3 [M+H]⁺.

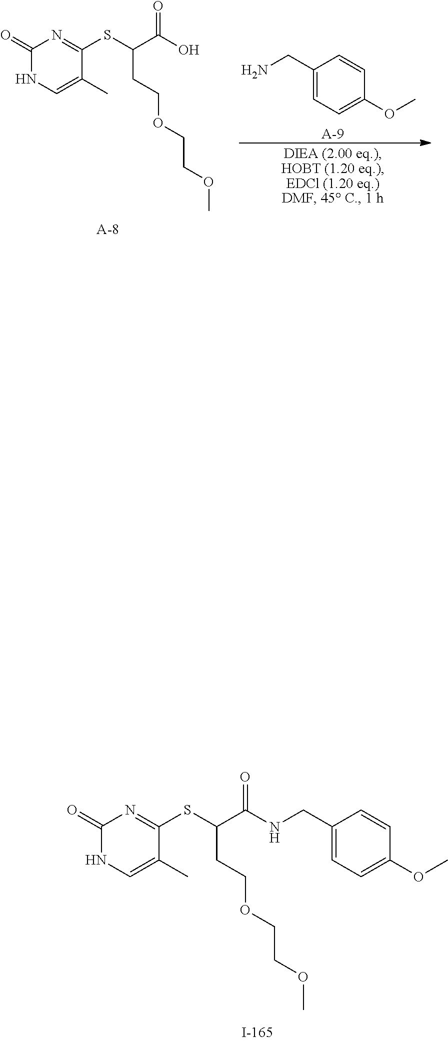

A mixture of 4-(2-methoxyethoxy)-2-[(5-methyl-2-oxo-1H-pyrimidin-4-yl)sulfanyl]butanoic acid (0.06 g, 198 μmol, 1.00 eq.), (4-methoxyphenyl)methanamine (27.2 mg, 198 μmol, 25.7 μL, 1.00 eq.), DIEA (51.3 mg, 396 μmol, 69.1 μL, 2.00 eq.), HOBt (32.1 mg, 238 mol, 1.20 eq.), EDCI (45.6 mg, 238 μmol, 1.20 eq.) in DMF (2.00 mL) was stirred at 45° C. for 1 h. LCMS showed 4-(2-methoxyethoxy)-2-[(5-methyl-2-oxo-1H-pyrimidin-4-yl)sulfanyl]butanoic acid was consumed, and one new peak with desired mass was detected. The mixture was concentrated to get the crude product. The crude product was purified by prep-HPLC (column: Phenomenex Luna C18 150*30 mm*5 um; mobile phase: [water(HCl)-ACN]; gradient: 15%-45% B over 14 min). Compound 4-(2-methoxyethoxy)-N-[(4-methoxyphenyl)methyl]-2-[(5-methyl-2-oxo-1H-pyrimidin-4-yl)sulfanyl]butanamide (0.035 g, 83.03 μmol, 41.84% yield) was obtained as white solid and confirmed by LCMS and ¹HNMR. LCMS: 422.4 [M+H]⁺. ¹H NMR: (400 MHz, DMSO-d6): δ 11.47 (br s, 1H), 8.65 (t, J=5.9 Hz, 1H), 7.53 (d, J=0.6 Hz, 1H), 7.20-7.14 (m, 2H), 6.89-6.83 (m, 2H), 4.62 (dd, J=6.3, 8.1 Hz, 1H), 4.21 (d, J=5.9 Hz, 2H), 3.72 (s, 3H), 3.46-3.39 (m, 6H), 3.22 (s, 3H), 2.18-2.07 (m, 1H), 2.01 (td, J=6.7, 13.4 Hz, 1H), 1.92 (s, 3H).

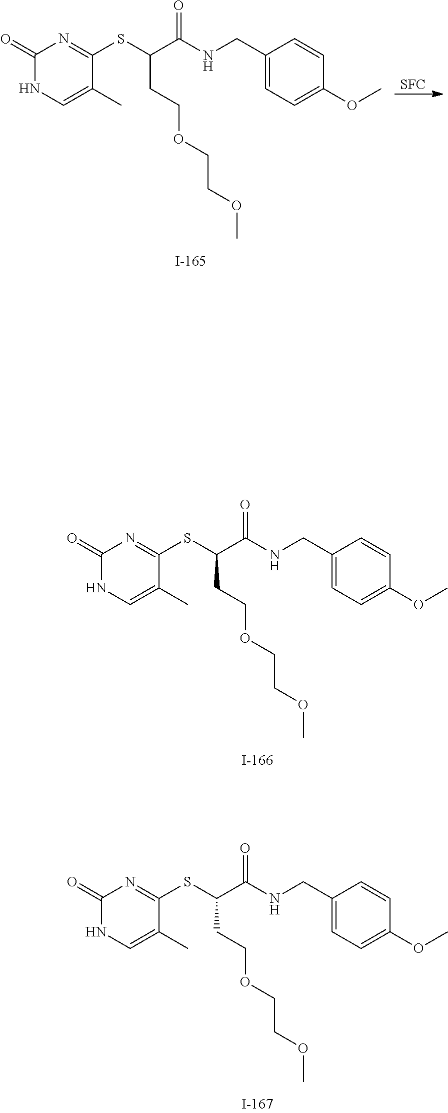

Compound 4-(2-methoxyethoxy)-N-[(4-methoxyphenyl)methyl]-2-[(5-methyl-2-oxo-1H-pyrimidin-4-yl)sulfanyl]butanamide (0.02 g, 47.4 μmol, 1.00 eq.) was purified by SFC (column: DAICEL CHIRALPAK AS (250 mm*30 mm, 10 um); mobile phase: [CO2-MeOH (0.1% NH3H2O)]; B %: 20%, isocratic elution mode). Compound (2R)-4-(2-methoxyethoxy)-N-[(4-methoxyphenyl)methyl]-2-[(5-methyl-2-oxo-1H-pyrimidin-4-yl)sulfanyl]butanamide (0.0075 g, 17.36 μmol, 36.59% yield, 97.568% purity) was obtained as yellow oil and confirmed by LCMS, ¹HNMR and SFC. LCMS: 422.1 [M+H]⁺. ¹HNMR: EC7336-464-P1C1 (400 MHz, CHLOROFORM-d): δ 12.35 (br s, 1H), 7.55 (br t, J=5.5 Hz, 1H), 7.15 (s, 1H), 7.10 (d, J=8.6 Hz, 2H), 6.70 (d, J=8.5 Hz, 2H), 4.69 (dd, J=6.5, 8.6 Hz, 1H), 4.37-4.21 (m, 2H), 3.65 (s, 3H), 3.61-3.53 (m, 1H), 3.51-3.41 (m, 5H), 3.27 (s, 3H), 2.37-2.27 (m, 1H), 2.09-2.00 (m, 1H), 1.95 (s, 3H).

Compound (2S)-4-(2-methoxyethoxy)-N-[(4-methoxyphenyl)methyl]-2-[(5-methyl-2-oxo-1H-pyrimidin-4-yl)sulfanyl]butanamide (0.0055 g, 11.82 μmol, 24.92% yield, 90.606% purity) was obtained as yellow oil and confirmed by LCMS, ¹HNMR and SFC. LCMS: 422.1 [M+H]⁺. ¹H NMR: EC7336-464-P1C2 (400 MHz, CHLOROFORM-d): δ 7.63 (br s, 1H), 7.22 (s, 1H), 7.17 (br d, J=8.6 Hz, 2H), 6.77 (d, J=8.6 Hz, 2H), 4.73 (t, J=7.8 Hz, 1H), 4.41-4.24 (m, 2H), 3.69 (s, 3H), 3.64-3.58 (m, 1H), 3.56-3.41 (m, 6H), 3.30 (s, 3H), 2.42-2.28 (m, 1H), 2.06 (br dd, J=7.1, 14.2 Hz, 1H), 1.97 (s, 3H).

### Example 7: Synthesis of Compounds I-201, I-202, I-462, I-487 and I-496

Compounds I-201, I-202, I-462, I-487 and I-496 were synthesized according to the schemes shown below.

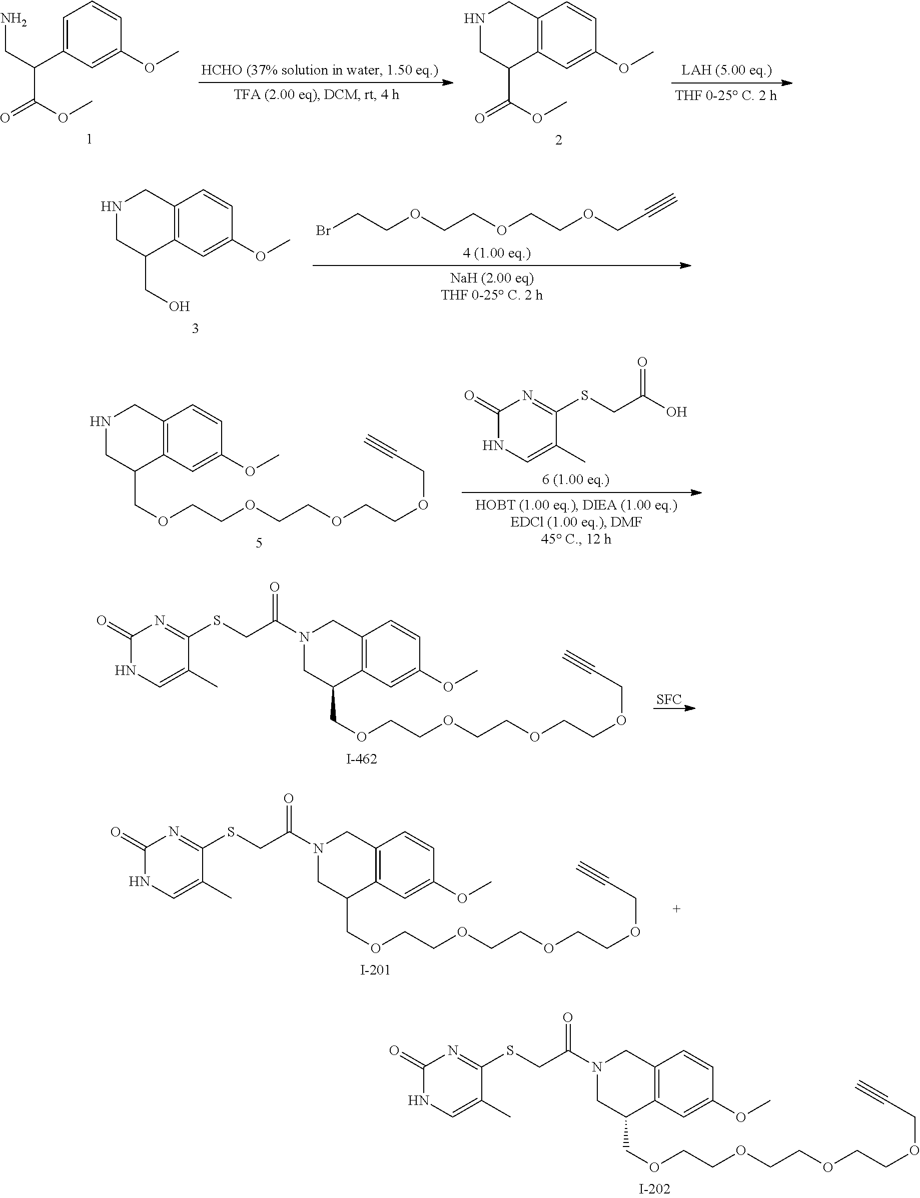

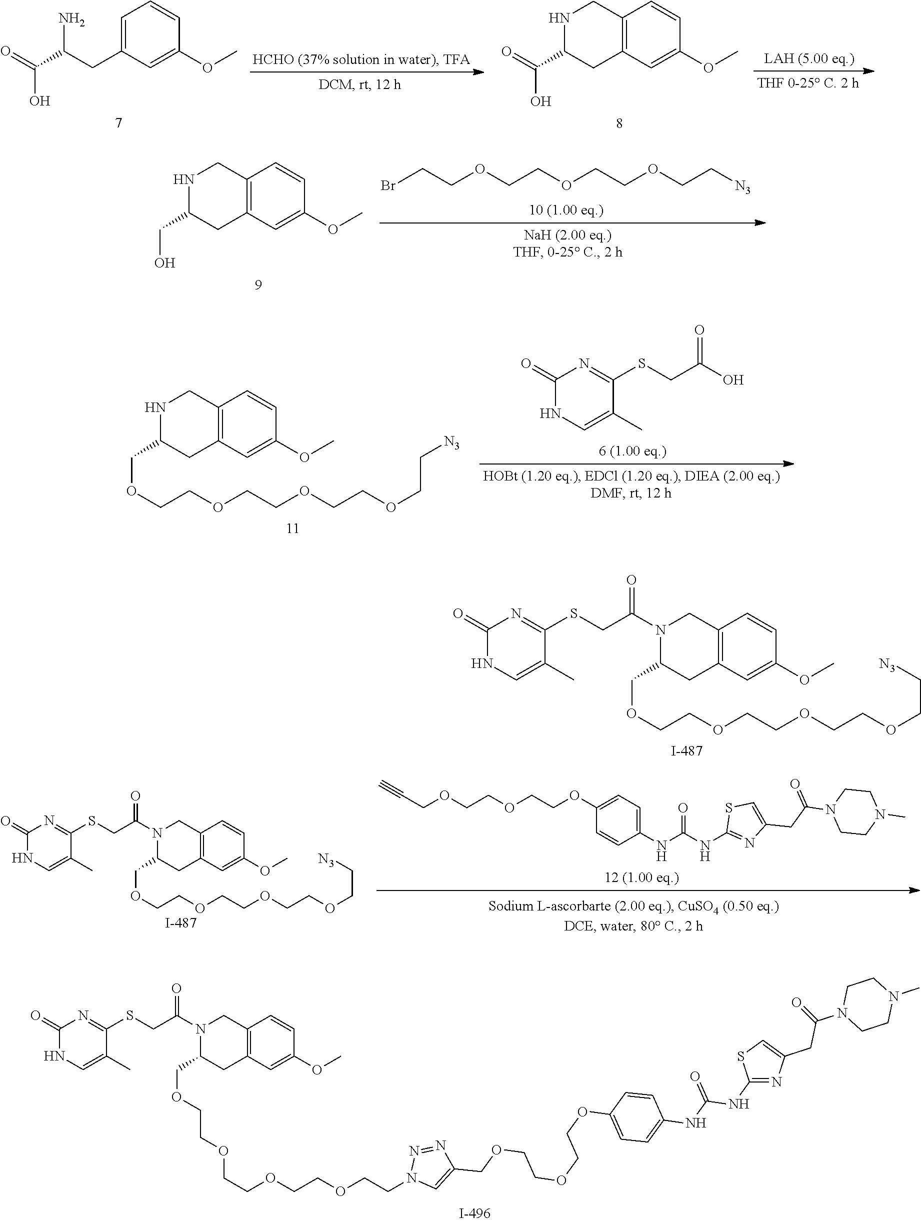

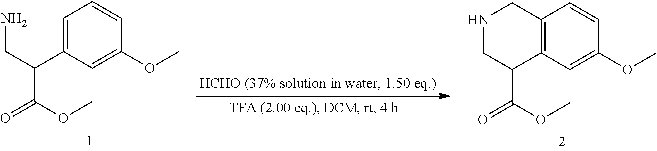 [SMILES: COC(=O)C(CN)c1cccc(OC)c1 ; COC(=O)C1CNCc2ccc(OC)cc21]

To a solution of methyl 3-amino-2-(3-methoxyphenyl)propanoate (4.00 g, 16.28 mmol, 1.00 eq. HCl) and formaldehyde (1.98 g, 24.42 mmol, 1.82 mL, 37% purity, 1.50 eq.) in DCM (50 mL) was added TFA (3.71 g, 32.56 mmol, 2.42 mL, 2.00 eq.) into above the mixture at 0° C. The mixture was stirred at 25° C. for 4 h. LCMS showed methyl 3-amino-2-(3-methoxyphenyl)propanoate was consumed completely and one main peak with desired mass was detected. TLC (Dichloromethane:Methanol=20:1, R_{f}=0.11) indicated methyl 3-amino-2-(3-methoxyphenyl)propanoate was consumed completely and two new spots formed. The reaction was clean according to TLC. The reaction mixture was concentrated under reduced pressure to remove DCM (50 mL) to give a crude product. The crude product was purified by prep-TLC (SiO2, DCM:MeOH=15:1). Compound methyl 6-methoxy-1,2,3,4-tetrahydroisoquinoline-4-carboxylate (4.5 g, 13.42 mmol, 82.44% yield, TFA) was obtained as a yellow oil. LCMS: 222.0 [M+H]⁺.

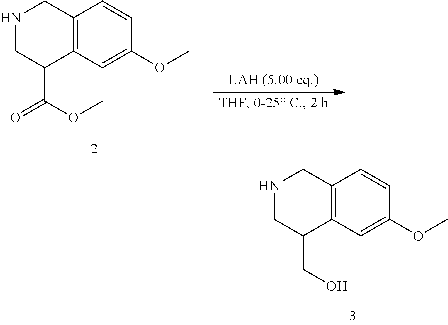 [SMILES: COC(=O)C1CNCc2ccc(OC)cc21 ; "LAH (5.00 eq.)" ; "THF, 0-25° C., 2 h" ; COc1ccc2c(c1)C(CO)CNC2]

To a solution of methyl 6-methoxy-1,2,3,4-tetrahydroisoquinoline-4-carboxylate (4 g, 11.93 mmol, 1.00 eq., TFA) in THF (20 mL) and LAH (2.5 M, 20 mL, 5.00 eq.) was added into above the mixture at 0° C. The mixture was stirred at 25° C. for 2 h. LCMS showed methyl 6-methoxy-1,2,3,4-tetrahydroisoquinoline-4-carboxylate was consumed completely and one main peak with desired mass was detected. The reaction mixture was quenched by addition NH₄Cl (2M) 40 mL at 0° C., diluted with MeOH 40 mL, and then filtered and concentrated under reduced pressure to give a crude product. The crude product was purified by prep-HPLC (column: Phenomenex luna C18 (250*70 mm, 10 um); mobile phase: [water(HCl)-ACN], gradient: 0%-10% B over 5 min). Compound (6-methoxy-1,2,3,4-tetrahydroisoquinolin-4-yl)methanol (1.35 g, 6.99 mmol, 58.56% yield) was obtained as a white solid. LCMS: 194.1 [M+H]⁺.

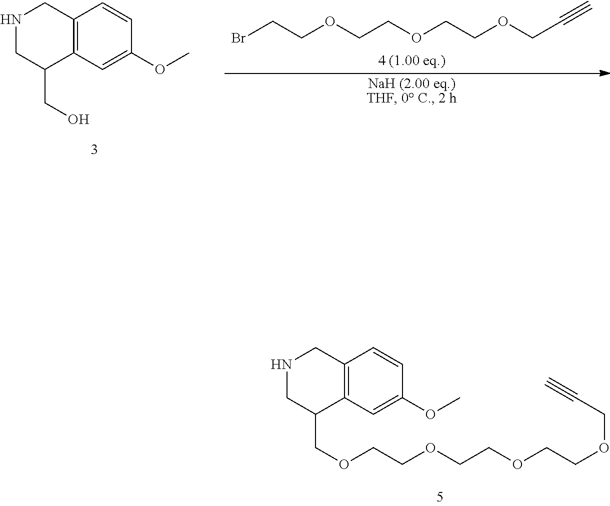

To a solution of (6-methoxy-1,2,3,4-tetrahydroisoquinolin-4-yl)methanol (150 mg, 776.23 μmol, 1.00 eq) in THF (2 mL) was added NaH (62.10 mg, 1.55 mmol, 60% purity, 2.00 eq). The mixture was stirred at 0° C. for 0.5 h under N2 atmosphere. 3-[2-[2-(2-bromoethoxy)ethoxy]ethoxy]prop-1-yne (194.92 mg, 776.23 μmol, 1.00 eq) was added into above mixture. The mixture was stirred at 0° C. for 1.5 h. LCMS showed (6-methoxy-1,2,3,4-tetrahydroisoquinolin-4-yl)methanol (150 mg, 776.23 μmol, 1.00 eq) was consumed and one main peak with desired mass was detected. The reaction mixture was quenched by addition H₂O 10 mL, and extracted with EtOAc (10 mL*3). The combined organic layers were concentrated under reduced pressure to give a crude product. The crude product was purified by prep-HPLC (column: 3_Phenomenex Luna C18 75*30 mm*3 um; mobile phase: [H2O (0.1% TFA)-ACN]; gradient: 10%-40% B over 8.0 min). Compound 6-methoxy-4-[2-[2-(2-prop-2-ynoxyethoxy)ethoxy]ethoxymethyl]-1,2,3,4-tetrahydroisoquinoline (75 mg, 206.36 μmol, 26.58% yield) was obtained as yellow oil. LCMS: 364.2 [M+H]⁺.

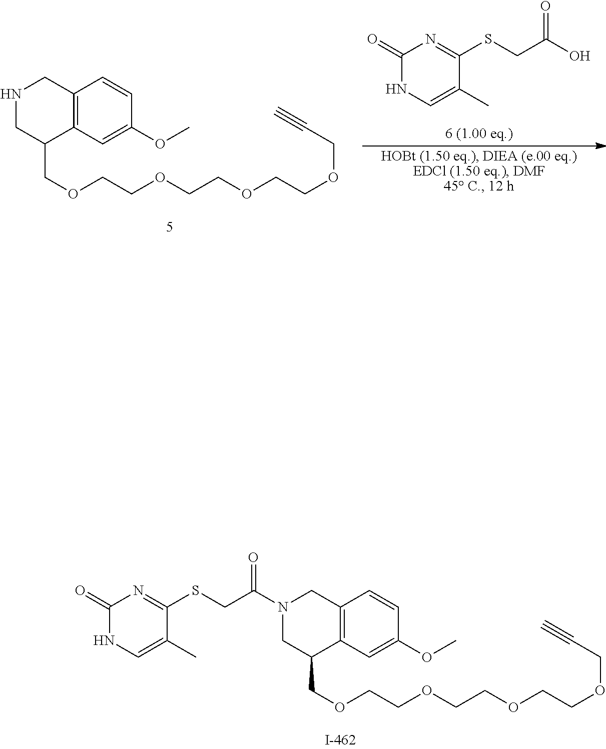

To a solution of 6-methoxy-3-[2-[2-(2-prop-2-ynoxyethoxy)ethoxy]ethoxymethyl]-1,2,3,4-tetrahydroisoquinoline (70 mg, 192.60 μmol, 1.00 eq.) and 2-[(5-methyl-2-oxo-1H-pyrimidin-4-yl)sulfanyl]acetic acid (38.56 mg, 192.60 μmol, 1.00 eq.) in DMF (2 mL) was added HOBt (39.04 mg, 288.90 μmol, 1.50 eq.), DIEA (74.68 mg, 577.80 μmol, 100.64 μL, 3.00 eq.) and EDCI (55.38 mg, 288.90 μmol, 1.50 eq.). The mixture was stirred at 45° C. for 1 h. LCMS showed 6-methoxy-3-[2-[2-(2-prop-2-ynoxyethoxy)ethoxy]ethoxymethyl]-1,2,3,4-tetrahydroisoquinoline was consumed and one main peak with desired mass was detected. The reaction mixture was concentrated under reduced pressure to remove DMF (2 mL). The crude product was purified by prep-HPLC (column: Waters Xbridge Prep OBD C18 150*40 mm*10 um; mobile phase: [H₂O (0.05% NH₃H₂O+10 mM NH₄HCO₃)-ACN]; gradient: 15%-45% B over 8.0 min). Compound 4-[2-[6-methoxy-4-[2-[2-(2-prop-2-ynoxyethoxy)ethoxy]ethoxymethyl]-3,4-dihydro-1H-isoquinolin-2-yl]-2-oxo-ethyl]sulfanyl-5-methyl-1H-pyrimidin-2-one (0.01 g, 18.33 μmol, 9.52% yield) was obtained as brown oil. The structure was confirmed by LCMS, ¹HNMR and SFC. LCMS: 546.2 [M+H]⁺. ¹H NMR: ET22502-1592-P1a1_HNMR (400 MHz, DMSO-d6): δ 7.50 (s, 1H), 7.12 (br d, J=8.5 Hz, 1H), 6.89-6.86 (m, 1H), 6.82 (br dd, J=2.6, 8.4 Hz, 1H), 6.11-5.95 (m, 1H), 5.01-4.87 (m, 1H), 4.27 (br d, J=2.7 Hz, 2H), 4.25-4.17 (m, 2H), 4.12 (d, J=2.4 Hz, 2H), 3.74 (s, 3H), 3.61 (br d, J=4.2 Hz, 2H), 3.54 (br s, 4H), 3.51 (br s, 4H), 3.48 (br s, 2H), 3.43-3.41 (m, 2H), 3.21-3.01 (m, 3H), 1.98-1.95 (m, 3H).

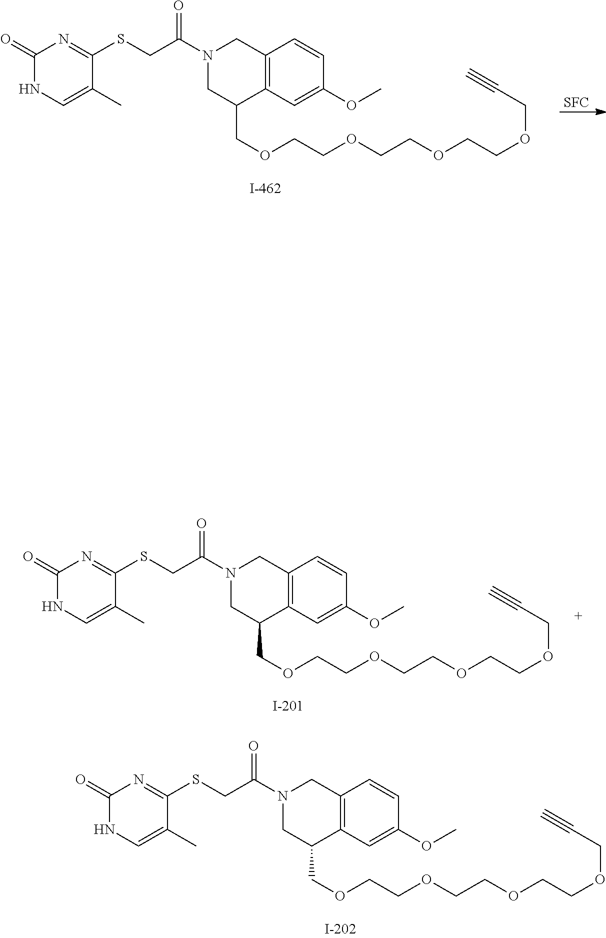

4-[2-[6-methoxy-4-[2-[2-(2-prop-2-ynoxyethoxy)ethoxy]ethoxymethyl]-3,4-dihydro-1H-isoquinolin-2-yl]-2-oxo-ethyl]sulfanyl-5-methyl-1H-pyrimidin-2-one was purified by SFC (column: DAICEL CHIRALPAK IG (250 mm*30 mm, 10 um); mobile phase: [CO2-EtOH:ACN=4:1]; B %: 60%, isocratic elution mode). Compound 4-[2-[(3 S)-6-methoxy-3-[2-[2-(2-prop-2-ynoxyethoxy)ethoxy]ethoxymethyl]-3,4-dihydro-1H-isoquinolin-2-yl]-2-oxo-ethyl]sulfanyl-5-methyl-1H-pyrimidin-2-one (15 mg, 27.49 μmol, 14.27% yield) was obtained as brown oil. The structure was confirmed by LCMS and ¹HNMR. LCMS: 564.2 [M+H]⁺. ¹H NMR: (400 MHz, DMSO-d6): δ 11.37 (br s, 1H), 7.54-7.47 (m, 1H), 7.16-7.02 (m, 1H), 6.90-6.79 (m, 2H), 5.01-4.62 (m, 1H), 4.33-4.15 (m, 3H), 4.14-4.11 (m, 2H), 3.88-3.73 (m, 3H), 3.63-3.35 (m, 17H), 3.18-3.02 (m, 1H), 2.00-1.94 (m, 3H).

Compound 4-[2-[(3R)-6-methoxy-3-[2-[2-(2-prop-2-ynoxyethoxy)ethoxy]ethoxymethyl]-3,4-dihydro-1H-isoquinolin-2-yl]-2-oxo-ethyl]sulfanyl-5-methyl-1H-pyrimidin-2-one (7.5 mg, 13.75 μmol, 7.14% yield) was obtained as brown oil. The structure was confirmed by LCMS and ¹HNMR. LCMS: 564.3 [M+H]⁺. ¹H NMR: (400 MHz, DMSO-d6): δ 11.37 (br s, 1H), 7.50 (s, 1H), 7.12 (br d, J=8.5 Hz, 1H), 6.89-6.80 (m, 2H), 4.98 (br d, J=17.0 Hz, 1H), 4.33-4.10 (m, 6H), 3.74 (s, 3H), 3.61 (br d, J=4.4 Hz, 2H), 3.54-3.41 (m, 14H), 3.15 (br d, J=8.1 Hz, 1H), 2.00-1.92 (m, 3H).

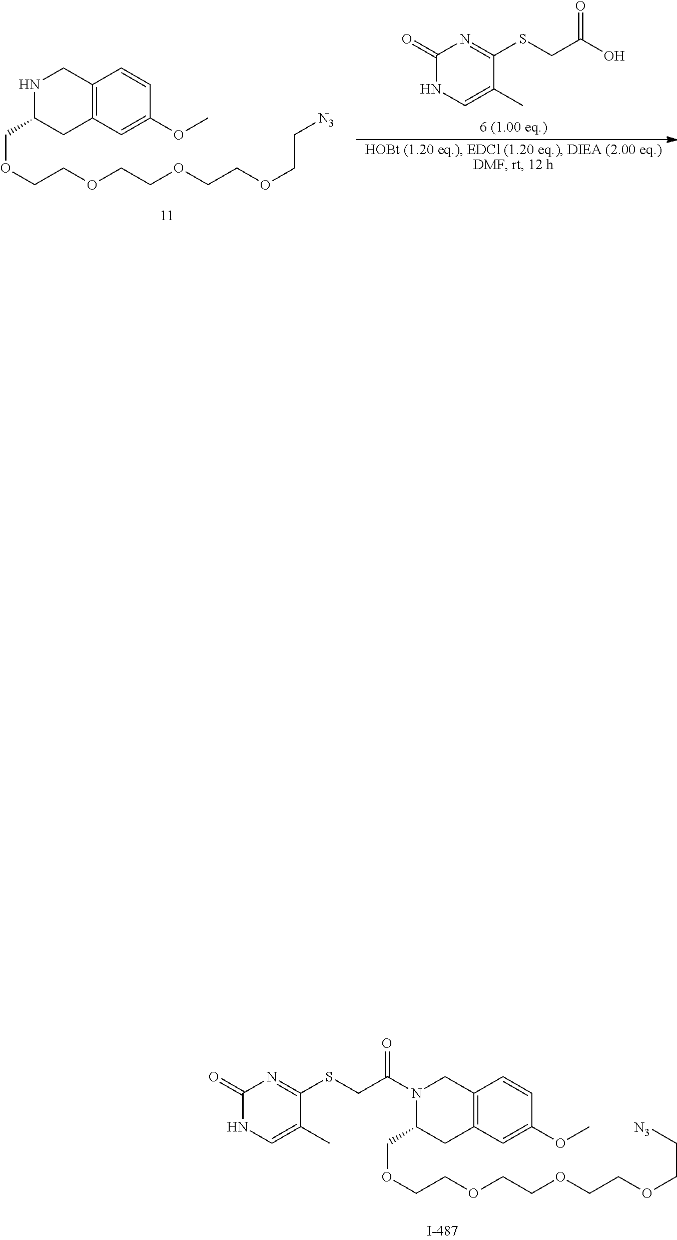

A mixture of (3R)-3-[2-[2-[2-(2-azidoethoxy)ethoxy]ethoxy]ethoxymethyl]-6-methoxy-1,2,3,4-tetrahydroisoquinoline (0.50 g, 1.27 mmol, 1.00 eq.), 2-[(5-methyl-2-oxo-1H-pyrimidin-4-yl)sulfanyl]acetic acid (253.78 mg, 1.27 mmol, 1.00 eq.), DIEA (327.63 mg, 2.54 mmol, 441.56 μL, 2.00 eq.), HOBt (205.52 mg, 1.52 mmol, 1.20 eq.), EDCI (291.59 mg, 1.52 mmol, 1.20 eq.) in DMF (3 mL) was stirred at 45° C. for 1 h. LCMS showed (3R)-3-[2-[2-[2-(2-azidoethoxy)ethoxy]ethoxy]ethoxymethyl]-6-methoxy-1,2,3,4-tetrahydroisoquinoline was consumed, and one new peak with desired mass was detected. The mixture was concentrated to get the crude product. The crude product was purified by prep-HPLC (column: Waters xbridge 150*25 mm 10 um; mobile phase: [water(NH₄HCO₃)-ACN]; gradient: 20%-50% B over 10 min). Compound 4-[2-[(3R)-3-[2-[2-[2-(2-azidoethoxy)ethoxy]ethoxy]ethoxymethyl]-6-methoxy-3,4-dihydro-1H-isoquinolin-2-yl]-2-oxo-ethyl]sulfanyl-5-methyl-1H-pyrimidin-2-one (0.30 g, 506.62 μmol, 39.97% yield, 97.383% purity) was obtained as purple oil which was confirmed by LCMS, HPLC, SFC and ¹HNMR. LCMS: 577.2 [M+H]⁺. ¹H NMR: (400 MHz, METHANOL-d4): δ 7.48-7.44 (m, 1H), 7.17-7.07 (m, 1H), 6.83-6.74 (m, 2H), 4.92 (br d, J=2.4 Hz, 1H), 4.72-4.62 (m, 1H), 4.39 (s, 1H), 4.29 (d, J=16.0 Hz, 1H), 3.81-3.78 (m, 3H), 3.67-3.57 (m, 14H), 3.50-3.44 (m, 2H), 3.37 (t, J=4.9 Hz, 2H), 3.30-3.24 (m, 1H), 2.99 (br d, J=3.0 Hz, 1H), 2.87 (br d, J=15.4 Hz, 1H), 2.10 (s, 3H).

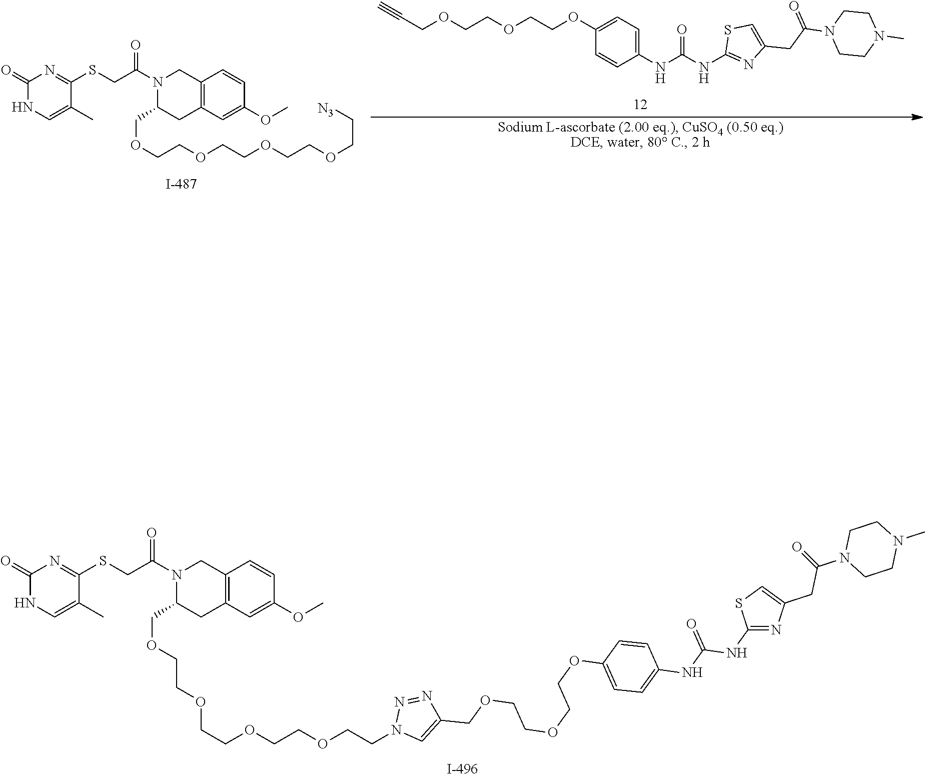 [SMILES: COc1ccc2c(c1)C[C@H](COCCOCCOCCOCCN=[N+]=[N-])N(C(=O)CSc1nc(=O)[nH]cc1C)C2 ; C#CCOCCOCCOc1ccc(NC(=O)Nc2nc(CC(=O)N3CCN(C)CC3)cs2)cc1 ; "12" ; "Sodium L-ascorbate (2.00 eq.), CuSO4 (0.50 eq.)DCE, water, 80° C., 2 h" ; COc1ccc2c(c1)C[C@H](COCCOCCOCCOCCn1cc(COCCOCCOc3ccc(NC(=O)Nc4nc(CC(=O)N5CCN(C)CC5)cs4)cc3)nn1)N(C(=O)CSc1nc(=O)[nH]cc1C)C2]

A mixture of 4-[2-[(3R)-3-[2-[2-[2-(2-azidoethoxy)ethoxy]ethoxy]ethoxymethyl]-6-methoxy-3,4-dihydro-1H-isoquinolin-2-yl]-2-oxo-ethyl]sulfanyl-5-methyl-1H-pyrimidin-2-one (0.05 g, 86.71 μmol, 1.00 eq.), 1-[4-[2-(4-methylpiperazin-1-yl)-2-oxo-ethyl]thiazol-2-yl]-3-[4-[2-(2-prop-2-ynoxyethoxy)ethoxy]phenyl]urea (43.49 mg, 86.71 μmol, 1.00 eq.), CuSO₄.5H₂O (10.82 mg, 43.35 μmol, 0.50 eq.) and sodium; (2R)-2-[(1S)-1,2-dihydroxyethyl]-4-hydroxy-5-oxo-2H-furan-3-olate (34.35 mg, 173.41 μmol, 2.00 eq.) in DCE (6 mL) and H₂O (6 mL) was stirred at 80° C. for 2 h. LC-MS showed 9.507% of 4-[2-[(3R)-3-[2-[2-[2-(2-azidoethoxy)ethoxy]ethoxy]ethoxymethyl]-6-methoxy-3,4-dihydro-1H-isoquinolin-2-yl]-2-oxo-ethyl]sulfanyl-5-methyl-1H-pyrimidin-2-one remained and one main peak with desired mass was detected. The reaction mixture was extracted with CH₂Cl₂ 60 ml (10 ml*6). The combined organic layers were concentrated under reduced pressure to give a crude product. The crude product was purified by prep-TLC (SiO₂, Dichloromethane:Methanol=5:1). Compound 1-[4-[2-[2-[[1-[2-[2-[2-[2-[[(3R)-6-methoxy-2-[2-[(5-methyl-2-oxo-1H-pyrimidin-4-yl)sulfanyl]acetyl]-3,4-dihydro-1H-isoquinolin-3-yl]methoxy]ethoxy]ethoxy]ethoxy]ethyl]triazol-4-yl]methoxy]ethoxy]ethoxy]phenyl]-3-[4-[2-(4-methylpiperazin-1-yl)-2-oxo-ethyl]thiazol-2-yl]urea (12 mg, 10.66 μmol, 12.30% yield, 95.82% purity) was obtained as yellow oil which was confirmed by LCMS and ¹HNMR. LCMS: 1078.3 [M+H]⁺. ¹H NMR: (400 MHz, DMSO-d6): δ 11.92-11.17 (m, 1H), 11.06-10.35 (m, 1H), 9.15 (s, 1H), 8.19 (d, J=13.5 Hz, 1H), 7.73-7.57 (m, 1H), 7.48 (d, J=9.2 Hz, 2H), 7.31-7.16 (m, 1H), 7.00 (d, J=9.2 Hz, 2H), 6.92-6.77 (m, 3H), 4.93-4.80 (m, 1H), 4.75-4.60 (m, 2H), 4.58-4.43 (m, 5H), 4.32-4.04 (m, 4H), 3.87-3.79 (m, 2H), 3.76-3.72 (m, 4H), 3.69 (s, 2H), 3.61 (s, 4H), 3.54-3.51 (m, 4H), 3.49-3.44 (m, 11H), 3.19-3.08 (m, 2H), 2.88-2.71 (m, 2H), 2.30-2.20 (m, 4H), 2.16 (s, 3H), 2.03-1.89 (m, 4H).

### Example 8: Synthesis of Compounds I-421 and I-422

Compounds I-421 and 1-422 were synthesized according to the schemes shown below.

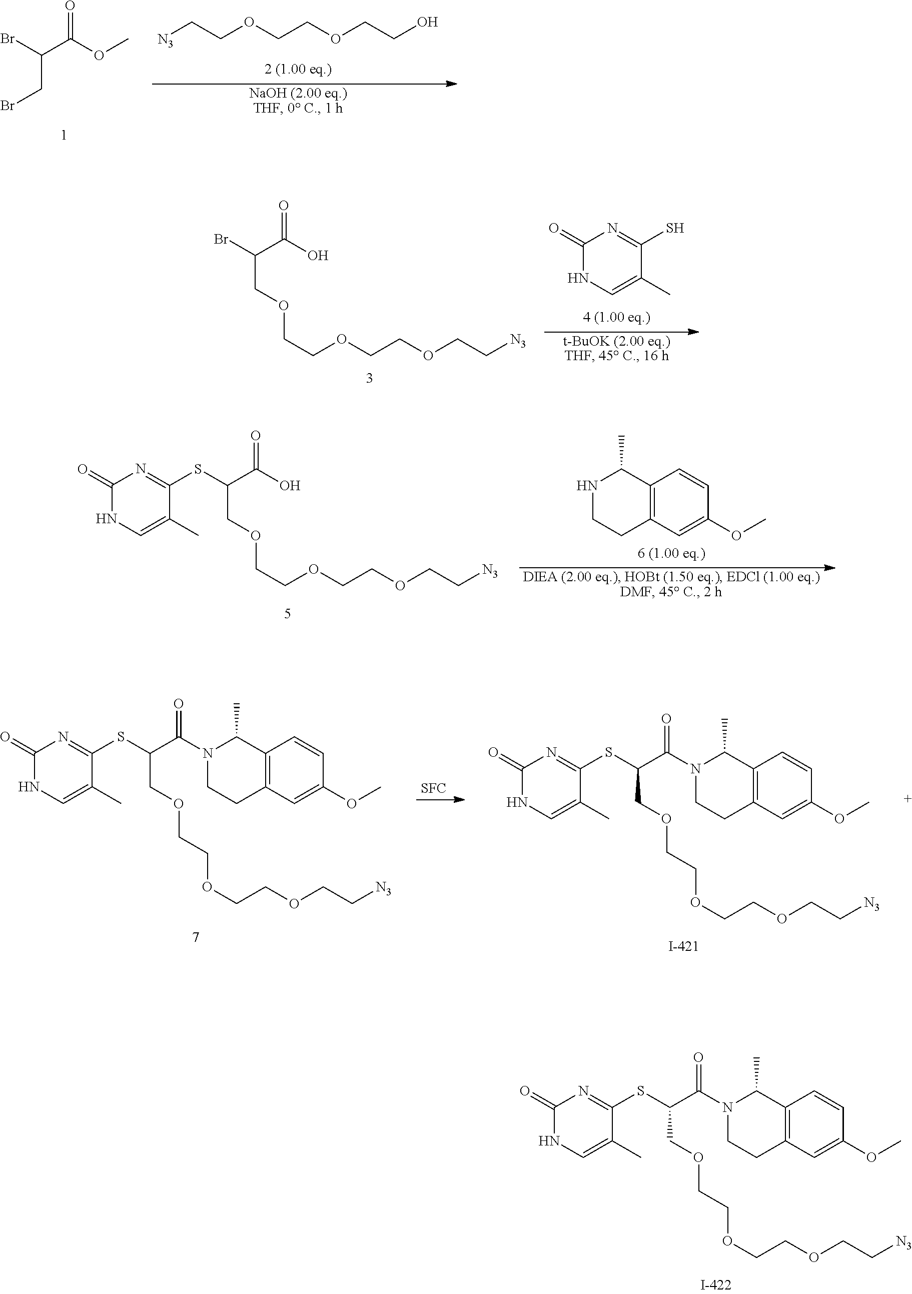

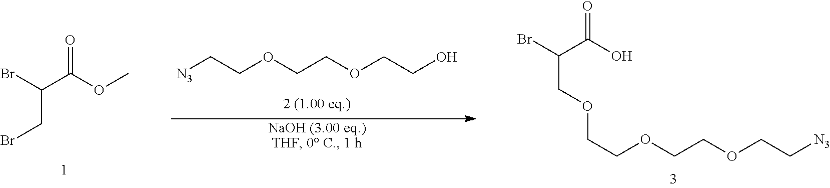

A mixture of methyl 2,3-dibromopropanoate (3.37 g, 13.70 mmol, 1.73 mL, 1.00 eq.), 2-[2-(2-azidoethoxy)ethoxy]ethanol (2.40 g, 13.70 mmol, 1.00 eq.), NaOH (1.64 g, 41.10 mmol, 3.00 eq.) in THF (36 mL) was degassed and purged with N₂ for 3 times, and then the mixture was stirred at 0° C. for 1 h under N₂ atmosphere. LC-MS showed methyl 2,3-dibromopropanoate was consumed completely and one main peak with desired mass was detected. The reaction mixture was filtered and the filtrate was concentrated under reduced pressure to give a crude product. The crude product was used to next step directly without further purification. Compound 3-[2-[2-(2-azidoethoxy)ethoxy]ethoxy]-2-bromo-propanoic acid (8 g, 12.26 mmol, 89.52% yield, 50% purity) was obtained as a white solid. LCMS: 324.0 [M+H]⁺.

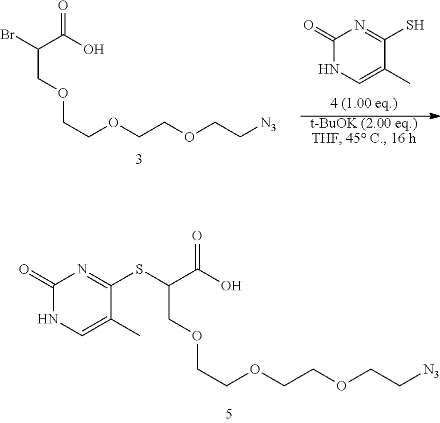

To a solution of 3-[2-[2-(2-azidoethoxy)ethoxy]ethoxy]-2-bromo-propanoic acid (8.00 g, 12.26 mmol, 1.00 eq.) in MeOH (60 mL) was added NaOH (1.47 g, 36.79 mmol, 3.00 eq.) and 5-methyl-4-sulfanyl-1H-pyrimidin-2-one (1.74 g, 12.26 mmol, 1.00 eq.). The mixture was stirred at 45° C. for 16 h. LC-MS showed 3-[2-[2-(2-azidoethoxy)ethoxy]ethoxy]-2-bromo-propanoic acid was consumed completely and one main peak with desired mass was detected. The reaction mixture was filtered and the filtrate was concentrated under reduced pressure to give a crude product. The crude product was purified by prep-HPLC (column: Phenomenex luna C18 (250*70 mm, 10 um); mobile phase: [water(NH₄HCO₃)-ACN]; gradient: 0%-25% B over 20 min). Compound 3-[2-[2-(2-azidoethoxy)ethoxy]ethoxy]-2-[(5-methyl-2-oxo-1H-pyrimidin-4-yl)sulfanyl]propanoic acid (0.80 g, 1.55 mmol, 12.63% yield, 75% purity) was obtained as yellow oil. MS: 388.1 [M+H]⁺.

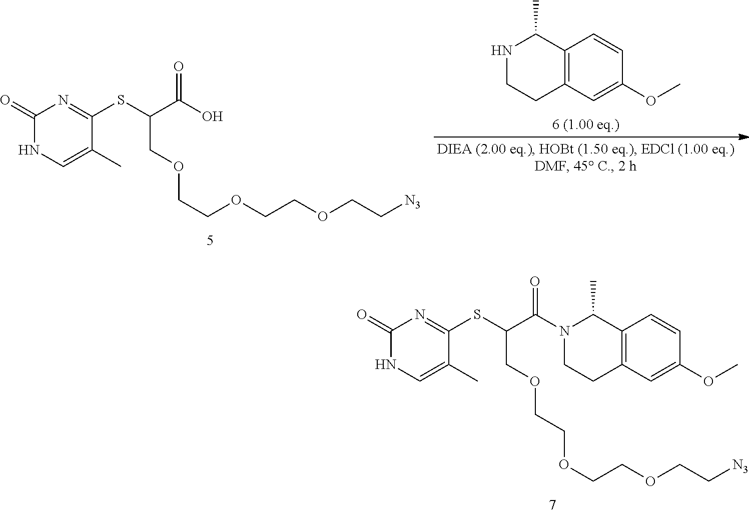 [SMILES: Cc1c[nH]c(=O)nc1SC(COCCOCCOCCN=[N+]=[N-])C(=O)O ; COc1ccc2c(c1)CCN[C@@H]2C ; COc1ccc2c(c1)CCN(C(=O)C(COCCOCCOCCN=[N+]=[N-])Sc1nc(=O)[nH]cc1C)[C@@H]2C]

To a solution of 3-[2-[2-(2-azidoethoxy)ethoxy]ethoxy]-2-[(5-methyl-2-oxo-1H-pyrimidin-4-yl)sulfanyl]propanoic acid (233.15 mg, 451.36 μmol, 1.00 eq.) in DMF (0.5 mL) was added DIEA (116.67 mg, 902.72 μmol, 157.24 μL, 2.00 eq.), HOBt (91.48 mg, 677.04 μmol, 1.50 eq.), EDCI (86.53 mg, 451.36 μmol, 1.00 eq.) and (1R)-6-methoxy-1-methyl-1,2,3,4-tetrahydroisoquinoline (0.08 g, 451.36 μmol, 1 eq.). The mixture was stirred at 45° C. for 2 h. LC-MS showed 3-[2-[2-(2-azidoethoxy)ethoxy]ethoxy]-2-[(5-methyl-2-oxo-1H-pyrimidin-4-yl)sulfanyl]propanoic acid was consumed completely and one main peak with desired mass was detected. The reaction mixture was quenched by addition H₂O 1 mL at 25° C. The crude product was purified by prep-HPLC (column: Phenomenex C18 150*25 mm*10 um; mobile phase: [water(NH₄HCO₃)-ACN]; gradient: 28%-58% B over 11 min). Compound 4-[1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one (0.04 g, 73.17 μmol, 16.21% yield) was obtained as a red solid. LCMS: 547.1 [M+H]⁺.

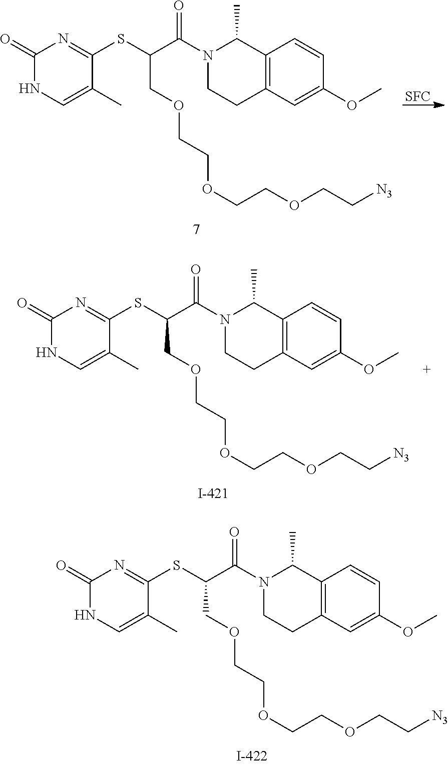

4-[1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one was separated by SFC (column: DAICEL CHIRALPAK AD (250 mm*30 mm, 10 um); mobile phase: [CO₂-i-PrOH (0.1% NH₃H₂O)]; B %: 40%, isocratic elution mode) to give the desired product 4-[(1R)-1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one (4 mg, 7.32 μmol, 10.00% yield) and 4-[(1S)-1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one (9.00 mg, 16.46 μmol, 22.50% yield). Compound 4-[(1R)-1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one (4.00 mg, 7.32 μmol, 10.00% yield) was obtained as a white solid which was confirmed by LCMS, ¹HNMR and SFC. LCMS: 547.2 [M+H]⁺. ¹H NMR: EC10958-335-P1A (400 MHz, DMSO-d6): δ 11.52 (br s, 1H), 7.57 (br d, J=7.0 Hz, 1H), 7.21-7.10 (m, 1H), 6.90-6.64 (m, 2H), 5.51-5.21 (m, 2H), 4.39-4.08 (m, 1H), 3.88 (br t, J=9.3 Hz, 1H), 3.76-3.65 (m, 4H), 3.60-3.50 (m, 6H), 3.51-3.38 (m, 2H), 3.36 (br s, 3H), 3.29-3.06 (m, 2H), 2.94-2.70 (m, 2H), 2.07-1.81 (m, 3H), 1.44-1.27 (m, 3H).

Compound 4-[(1S)-1-[2-[2-(2-azidoethoxy)ethoxy]ethoxymethyl]-2-[(1R)-6-methoxy-1-methyl-3,4-dihydro-1H-isoquinolin-2-yl]-2-oxo-ethyl]sulfanyl-5-methyl-1H-pyrimidin-2-one (9 mg, 16.46 μmol, 22.50% yield) was obtained as a white solid which was confirmed by LCMS, ¹HNMR and SFC. LCMS: 547.3 [M+H]⁺. ¹H NMR: EC10958-335-P2B (400 MHz, DMSO-d6): δ 11.55 (br s, 1H), 7.61-7.53 (m, 1H), 7.19-7.00 (m, 1H), 6.83-6.62 (m, 2H), 5.47-5.27 (m, 2H), 4.35-3.98 (m, 1H), 3.97-3.81 (m, 1H), 3.76-3.67 (m, 3H), 3.65-3.48 (m, 8H), 3.48-3.37 (m, 5H), 3.28-3.10 (m, 1H), 2.85 (br s, 2H), 1.94-1.80 (m, 3H), 1.30 (d, J=6.8 Hz, 3H).

### Example 9: Synthesis of Compounds I-280, I-358, I-281 and I-359

Compounds I-280, 1-358, 1-281 and 1-359 were synthesized according to the schemes shown below.

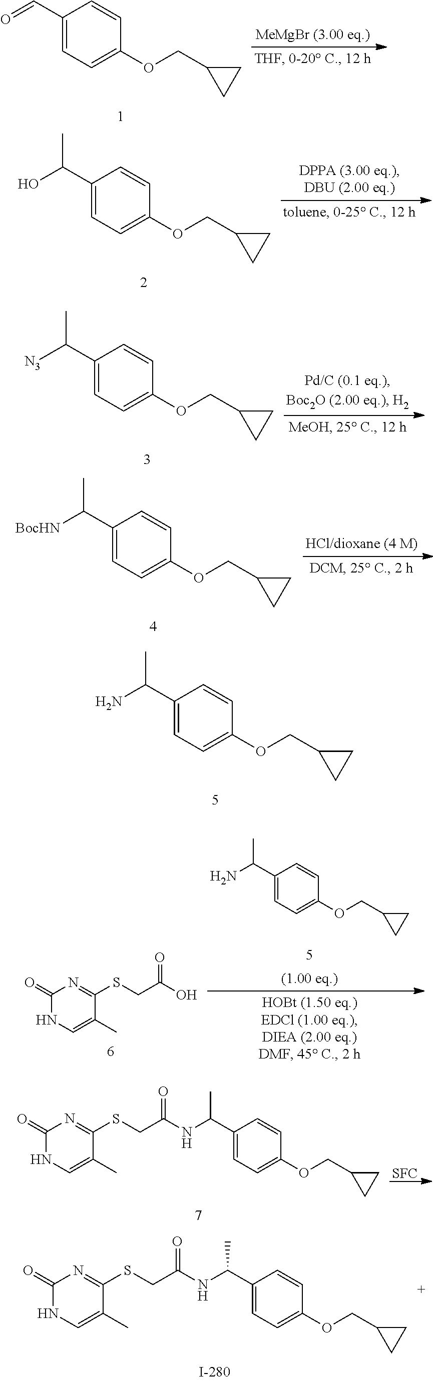

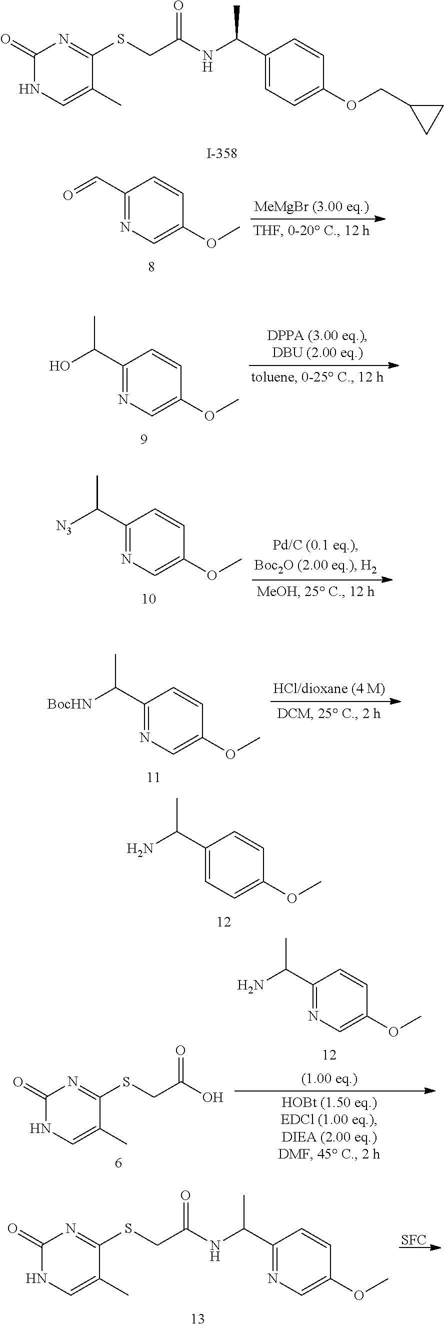

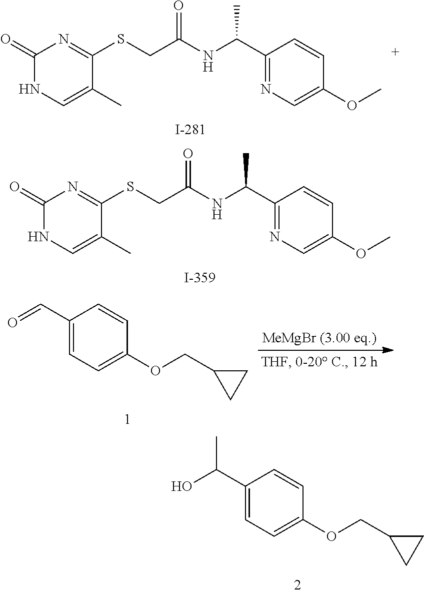

A mixture of 4-(cyclopropylmethoxy)benzaldehyde (3.00 g, 17.03 mmol, 1.00 eq.) in THF (30 mL) was degassed and purged with N₂ for 3 times, and then MeMgBr (3 M, 17.03 mL, 3.00 eq.) was added dropwise at −78° C. The resulting mixture was stirred at −78° C. for 1 h. TLC indicated 0% of 4-(cyclopropylmethoxy)benzaldehyde was remained, and one major new spot with larger polarity was detected (PE/EA=3/1). The crude product was extracted with NH₄Cl 30 mL and EtOAc (3*30 mL). The combined organic layers were washed with brine 20 mL, dried over by Na₂SO₄, filtered and concentrated under reduced pressure to give a crude product. The product was purified by prep-TLC (SiO₂, Petroleum ether:Ethyl acetate=3:1). Compound 1-[4-(cyclopropylmethoxy)phenyl]ethanol (2.88 g, 14.98 mmol, 87.99% yield) was obtained as a white solid. The structure was confirmed by ¹HNMR. ¹H NMR: (400 MHz, CHLOROFORM-d): δ 7.30 (d, J=8.6 Hz, 2H), 6.93-6.86 (m, 2H), 4.86 (q, J=6.5 Hz, 1H), 3.81 (d, J=6.9 Hz, 2H), 1.81-1.65 (m, 1H), 1.49 (d, J=6.5 Hz, 3H), 1.33-1.22 (m, 1H), 0.71-0.60 (m, 2H), 0.42-0.29 (m, 2H).

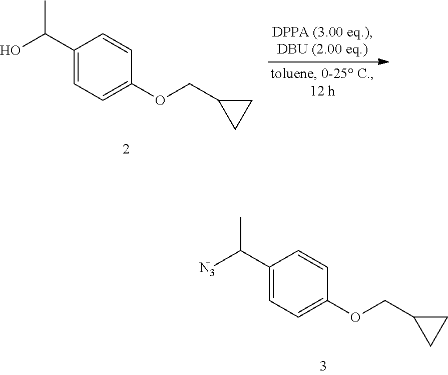 [SMILES: CC(O)c1ccc(OCC2CC2)cc1 ; CC(N=[N+]=[N-])c1ccc(OCC2CC2)cc1]

To a solution of 1-[4-(cyclopropylmethoxy)phenyl]ethanol (2.00 g, 10.40 mmol, 1.00 eq.) in toluene (20 mL) was added dropwise DPPA (8.59 g, 31.21 mmol, 6.74 mL, 3.00 eq.) and DBU (3.17 g, 20.81 mmol, 3.14 mL, 2.00 eq.) at 0° C. The resulting mixture was stirred at 25° C. for 12 h. TLC indicated 0% of 1-[4-(cyclopropylmethoxy)phenyl]ethanol was remained, and one major new spot with larger polarity was detected (PE/EA=10/1). The crude product was extracted with EtOAc (3*20 mL). The combined organic layers were dried over Na₂SO₄, filtered and concentrated under reduced pressure to give a crude product. The crude product was purified by prep-TLC (SiO₂, Petroleum ether:Ethyl acetate=10:1). Compound 1-(1-azidoethyl)-4-(cyclopropylmethoxy)benzene (2.1 g, 9.67 mmol, 92.91% yield) was obtained as white oil and confirmed by ¹HNMR. ¹H NMR: (400 MHz, CHLOROFORM-d): δ 7.22-7.13 (m, 2H), 6.87-6.77 (m, 2H), 4.05 (q, J=7.1 Hz, 1H), 3.73 (d, J=7.0 Hz, 2H), 1.97 (s, 1H), 1.43 (d, J=6.9 Hz, 3H), 1.25-1.15 (m, 2H), 0.62-0.54 (m, 2H), 0.32-0.25 (m, 2H).

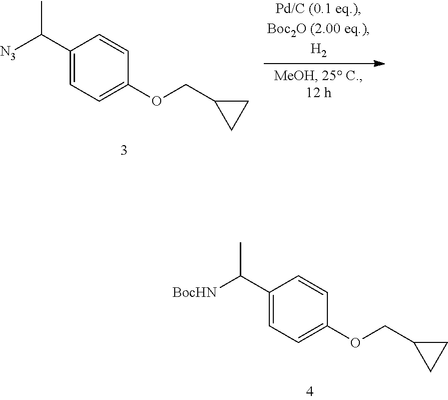 [SMILES: CC(N=[N+]=[N-])c1ccc(OCC2CC2)cc1 ; CC(NC(=O)OC(C)(C)C)c1ccc(OCC2CC2)cc1]

A mixture of 1-(1-azidoethyl)-4-(cyclopropylmethoxy)benzene (2.00 g, 9.21 mmol, 1.00 eq.), tert-butoxycarbonyl tert-butyl carbonate (4.02 g, 18.41 mmol, 4.23 mL, 2.00 eq.) and Pd/C (979.63 mg, 920.53 μmol, 10% purity, 0.10 eq.) in MeOH (20 mL) was degassed and purged with H₂ for 3 times, and then the mixture was stirred at 25° C. for 12 h under H₂ atmosphere. LC-MS showed 1-(1-azidoethyl)-4-(cyclopropylmethoxy)benzene was consumed completely and one main peak with desired mass was detected. The reaction mixture was filtered and the filtrate was concentrated under reduced pressure to give a crude product. The crude product was purified by column chromatography (SiO₂, Petroleum ether/Ethyl acetate=3/1 to 1/1). Compound tert-butyl N-[1-[4-(cyclopropylmethoxy)phenyl]ethyl]carbamate (0.88 g, 3.02 mmol, 32.81% yield) was obtained as a white solid. The structure was confirmed by ¹HNMR. LCMS: 583.3 [2M+H]⁺. ¹H NMR: (400 MHz, CHLOROFORM-d): δ 7.03 (d, J=8.6 Hz, 2H), 6.71-6.64 (m, 2H), 4.54 (br s, 2H), 3.60 (d, J=7.0 Hz, 2H), 1.24 (s, 12H), 1.12-1.03 (m, 1H), 0.49-0.42 (m, 2H), 0.19-0.13 (m, 2H).

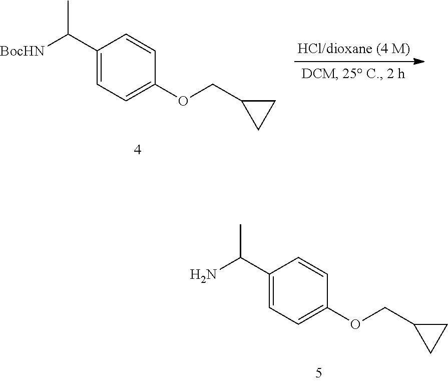 [SMILES: CC(NC(=O)OC(C)(C)C)c1ccc(OCC2CC2)cc1 ; CC(N)c1ccc(OCC2CC2)cc1]

To a solution of tert-butyl N-[1-[4-(cyclopropylmethoxy)phenyl]ethyl]carbamate (0.15 g, 514.78 μmol, 1.00 eq.) in DCM (2 mL) was added HCl/dioxane (4 M, 2 mL). TLC indicated 0% of tert-butyl N-[1-[4-(cyclopropylmethoxy)phenyl]ethyl]carbamate was remained, and one major new spot with larger polarity was detected (PE/EA=3/1). The reaction mixture was concentrated under reduced pressure to give a crude product. Compound 1-[4-(cyclopropylmethoxy)phenyl]ethanamine (0.09 g, 470.54 μmol, 91.41% yield) was obtained as a white solid.

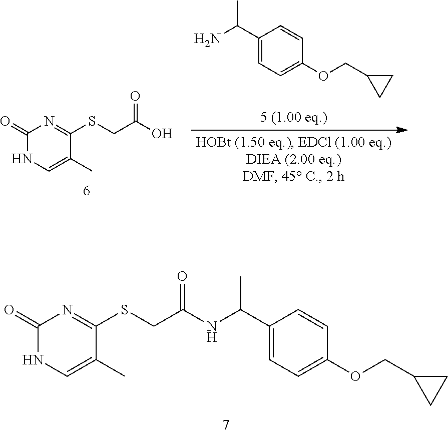 [SMILES: Cc1c[nH]c(=O)nc1SCC(=O)O ; CC(N)c1ccc(OCC2CC2)cc1 ; Cc1c[nH]c(=O)nc1SCC(=O)NC(C)c1ccc(OCC2CC2)cc1]

A mixture of 2-[(5-methyl-2-oxo-1H-pyrimidin-4-yl)sulfanyl]acetic acid (104.68 mg, 522.82 μmol, 1.00 eq.), 1-[4-(cyclopropylmethoxy)phenyl]ethanamine (0.1 g, 522.82 μmol, 1.00 eq.), HOBt (105.97 mg, 784.24 μmol, 1.50 eq.), EDCI (100.23 mg, 522.82 μmol, 1.00 eq.) and DIEA (135.14 mg, 1.05 mmol, 182.13 μL, 2.00 eq.) in DMF (1 mL), and then the mixture was stirred at 45° C. for 2 h. LC-MS showed 1-[4-(cyclopropylmethoxy)phenyl]ethanamine was consumed completely and one main peak with desired mass was detected. The reaction mixture was filtered and the filtrate was concentrated under reduced pressure to give a crude product. The crude product was purified by prep-HPLC (column: Welch Ultimate C18 150*25 mm*5 um; mobile phase: [water(FA)-ACN]; gradient: 23%-53% B over 10 min). Compound N-[(1R)-1-[4-(cyclopropylmethoxy)phenyl]ethyl]-2-[(5-methyl-2-oxo-1H-pyrimidin-4-yl)sulfanyl]acetamide (0.09 g, 240.98 μmol, 46.09% yield) was obtained as a white powder. LCMS: 374.1 [M+H]⁺.

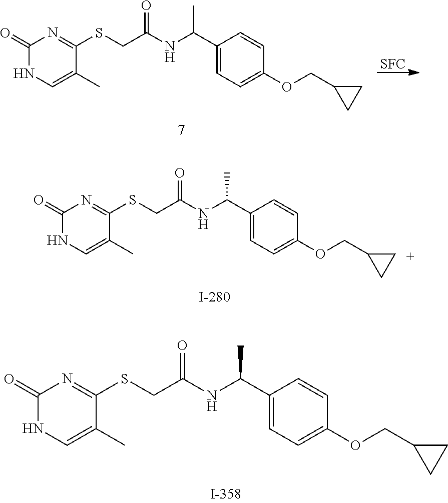

The product was separated by SFC (column: DAICEL CHIRALCEL OX (250 mm*30 mm, 10 um); mobile phase: [CO2-ACN/MeOH(0.1% NH3H2O)]; B %: 60%, isocratic elution mode). The structure was confirmed by HNMR. ¹H NMR: (400 MHz, DMSO-d6): δ 11.38 (br s, 1H), 8.76 (br d, J=7.9 Hz, 1H), 7.52 (s, 1H), 7.22 (d, J=8.5 Hz, 2H), 6.83 (d, J=8.6 Hz, 2H), 4.82 (t, J=7.3 Hz, 1H), 3.86 (d, J=3.9 Hz, 2H), 3.77 (d, J=6.9 Hz, 2H), 1.92 (s, 3H), 1.31 (d, J=6.9 Hz, 3H), 1.24 (br s, 1H), 0.59-0.53 (m, 2H), 0.30 (br d, J=5.6 Hz, 2H). ¹H NMR: (400 MHz, DMSO-d6): δ 11.35 (br s, 1H), 8.61 (d, J=7.9 Hz, 1H), 7.52 (s, 1H), 7.23 (d, J=8.6 Hz, 2H), 6.90-6.79 (m, 2H), 4.89-4.79 (m, 1H), 3.90 (d, J=2.0 Hz, 2H), 3.77 (d, J=6.9 Hz, 2H), 1.94 (s, 3H), 1.32 (d, J=7.0 Hz, 3H), 1.24 (br s, 1H), 0.59-0.53 (m, 2H), 0.30 (dd, J=1.5, 4.8 Hz, 2H).

### Example 10: Synthesis of Compounds I-331, I-195, I-196, I-332 and I-325

Compounds 1-331, 1-195, 1-196, 1-332 and 1-325 were synthesized according to the schemes shown below.

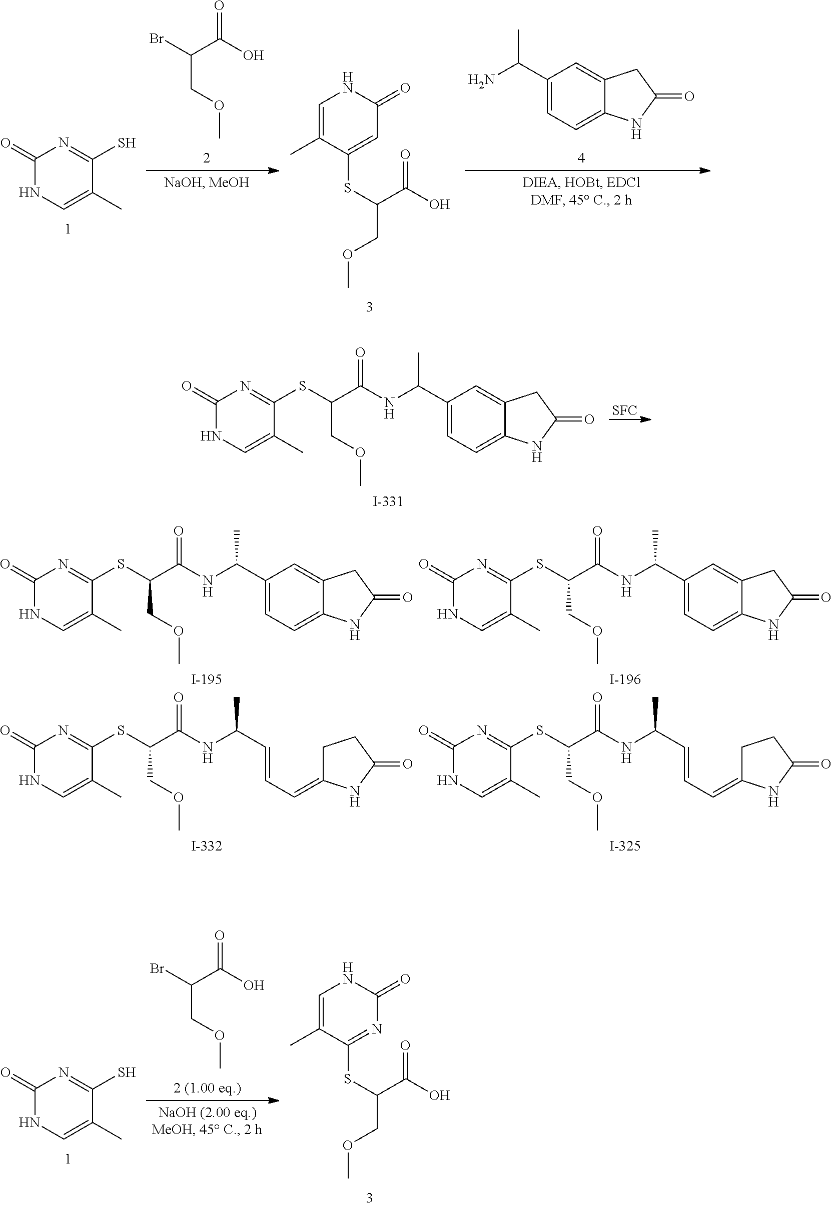

To a solution of 5-methyl-4-sulfanyl-1H-pyrimidin-2-one (2.00 g, 14.07 mmol, 1.00 eq.) in MeOH (10 mL) was added NaOH (1.13 g, 28.13 mmol, 2.00 eq.) and 2-bromo-3-methoxy-propanoic acid (2.57 g, 14.07 mmol, 1.00 eq.), after addition the mixture was stirred at 45° C. for 2 h. LC-MS showed 5-methyl-4-sulfanyl-1H-pyrimidin-2-one was consumed completely and one main peak with desired mass was detected. The reaction mixture was added HCl (1N in water) to pH=3, filtered and the filter cake was dried under reduced pressure to give a crude product. The crude product was used to next step directly without further purification. Compound 3-methoxy-2-[(5-methyl-2-oxo-1H-pyrimidin-4-yl)sulfanyl]propanoic acid (1.70 g, 6.96 mmol, 49.48% yield) was obtained as a yellow solid. LCMS: 244.9 [M+H]⁺.

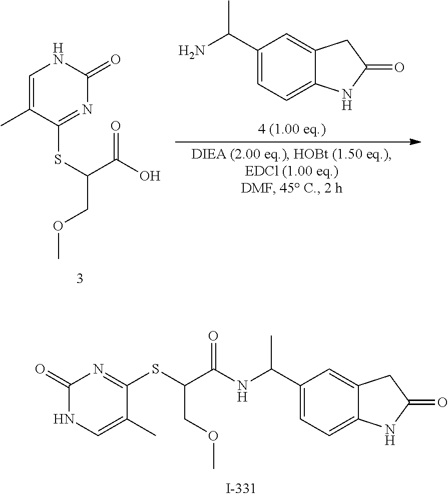 [SMILES: COCC(Sc1nc(=O)[nH]cc1C)C(=O)O ; CC(N)c1ccc2c(c1)CC(=O)N2 ; COCC(Sc1nc(=O)[nH]cc1C)C(=O)NC(C)c1ccc2c(c1)CC(=O)N2]

To a solution of 3-methoxy-2-[(5-methyl-2-oxo-1H-pyrimidin-4-yl)sulfanyl]propanoic acid (0.15 g, 614.08 μmol, 1.00 eq.) in DMF (0.5 mL) was added HOBt (124.47 mg, 921.12 μmol, 1.50 eq.), 5-(1-aminoethyl)indolin-2-one (108.21 mg, 614.08 μmol, 1.00 eq.), EDCI (117.72 mg, 614.08 μmol, 1.00 eq.) and DIEA (158.73 mg, 1.23 mmol, 213.92 μL, 2.00 eq.). The mixture was stirred at 45° C. for 2 h. LC-MS showed 3-methoxy-2-[(5-methyl-2-oxo-1H-pyrimidin-4-yl)sulfanyl]propanoic acid was consumed completely and one main peak with desired mass was detected. The reaction mixture was quenched by addition H₂O 1 mL at 25° C. The reaction mixture was purified by prep-HPLC (column: Waters xbridge 150*25 mm 10 um; mobile phase: [water(NH₄HCO₃)-ACN]; gradient: 88%-28% B over 14 min). Compound 3-methoxy-2-[(5-methyl-2-oxo-1H-pyrimidin-4-yl)sulfanyl]-N-[1-(2-oxoindolin-5-yl)ethyl]propanamide (0.08 g, 198.77 μmol, 32.37% yield) was obtained as a light yellow solid which was confirmed by LCMS and ¹HNMR. LCMS: 403.1 [M+H]. ¹HNMR: EC10958-228-P1R (400 MHz, DMSO-d6): δ 11.59-11.36 (m, 1H), 10.31 (br d, J=5.3 Hz, 1H), 8.84-8.77 (m, 1H), 7.54 (d, J=6.3 Hz, 1H), 7.19-7.07 (m, 2H), 6.76-6.69 (m, 1H), 4.89-4.76 (m, 2H), 3.73-3.52 (m, 2H), 3.46-3.40 (m, 2H), 3.26 (s, 3H), 1.91 (d, J=15.1 Hz, 3H), 1.34-1.29 (m, 3H).

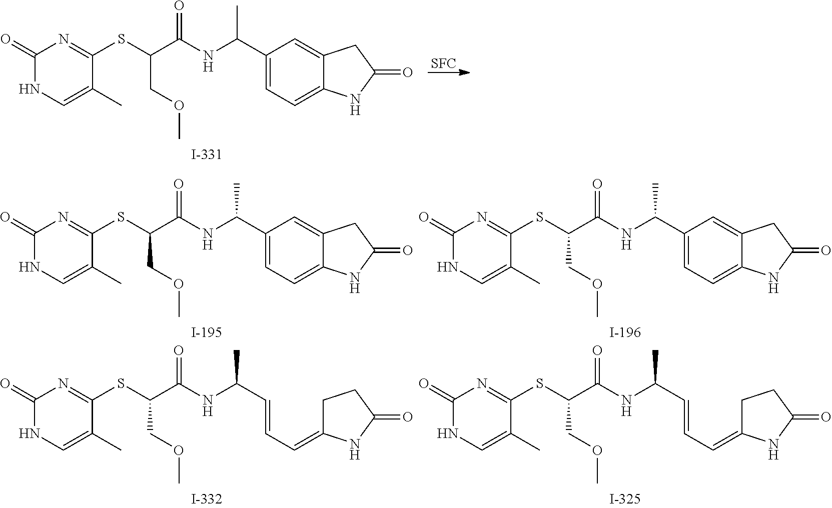

3-methoxy-2-[(5-methyl-2-oxo-1H-pyrimidin-4-yl)sulfanyl]-N-[1-(2-oxoindolin-5-yl)ethyl]propanamide was separated by SFC (column: DAICEL CHIRALPAK AS(250 mm*30 mm, 10 um); mobile phase: [CO₂-MeOH(0.1% NH₃H₂O)]; B %: 50%, isocratic elution mode). Compound (2R)-3-methoxy-2-[(5-methyl-2-oxo-1H-pyrimidin-4-yl)sulfanyl]-N-[(1R)-1-(2-oxoindolin-5-yl)ethyl]propanamide (0.0076 g, 18.88 μmol, 9.50% yield) was obtained as a white solid which was confirmed by LCMS, ¹HNMR and SFC. LCMS: 403.0 [M+H]⁺. ¹H NMR: EC10958-250-P1A1 (400 MHz, DMSO-d6): δ 10.31 (br s, 1H), 8.85 (br d, J=7.8 Hz, 1H), 7.54 (br s, 1H), 7.25-7.04 (m, 2H), 6.71 (br d, J=8.0 Hz, 1H), 4.89-4.73 (m, 2H), 3.74-3.64 (m, 2H), 3.30 (s, 3H), 3.25 (br s, 1H), 2.55 (s, 3H), 1.87 (s, 2H), 1.35-1.28 (m, 3H).

Compound (2S)-3-methoxy-2-[(5-methyl-2-oxo-1H-pyriimidin-4-yl)sulfanyl]-N-[(1R)-1-(2-oxoindolin-5-yl)ethyl]propanamide (0.0063 g, 15.65 μmol, 7.88% yield) was obtained as a white solid which was confirmed by LCMS, ¹HNMR and SFC. LCMS: 403.1 [M+H]⁺. ¹H NMR: EC10958-250-P2B1 (400 MHz, DMSO-d6): δ 10.32 (br s, 1H), 8.85 (br d, J=8.1 Hz, 1H), 7.56 (br s, 1H), 7.20-7.10 (m, 2H), 6.74 (d, J=8.0 Hz, 1H), 4.89-4.76 (m, 2H), 3.66-3.49 (m, 3H), 3.44 (br s, 2H), 3.25 (s, 3H), 1.91 (br s, 3H), 1.30 (br d, J=6.8 Hz, 3H).

Compound (2R)-3-methoxy-2-[(5-methyl-2-oxo-1H-pyrimidin-4-yl)sulfanyl]-N-[(1S)-1-(2-oxoindolin-5-yl)ethyl]propanamide (0.004 g, 9.94 μmol, 5.00% yield) was obtained as a white solid which was confirmed by LCMS, ¹HNMR and SFC. LCMS: 403.1 [M+H]⁺. ¹H NMR: EC10958-250-P3C (400 MHz, DMSO-d6): δ 10.38-10.18 (m, 1H), 8.85 (br d, J=8.0 Hz, 1H), 7.60-7.48 (m, 1H), 7.22-7.07 (m, 2H), 6.73 (d, J=8.0 Hz, 1H), 4.90-4.74 (m, 2H), 3.72-3.67 (m, 1H), 3.58-3.52 (m, 1H), 3.45-3.40 (m, 3H), 3.31 (s, 3H), 1.93-1.86 (m, 3H), 1.33-1.29 (m, 3H).

Compound (2S)-3-methoxy-2-[(5-methyl-2-oxo-1H-pyrimidin-4-yl)sulfanyl]-N-[(1S)-1-(2-oxoindolin-5-yl)ethyl]propanamide (0.016 g, 39.75 μmol, 20.00% yield) was obtained as brown oil which was confirmed by LCMS, ¹HNMR and SFC. MS: 403.1 [M+H]⁺. ¹H NMR: EC10958-250-P4C4 (400 MHz, DMSO-d6): δ 10.38 (br s, 1H), 8.89 (d, J=8.0 Hz, 1H), 7.61 (s, 1H), 7.25-7.13 (m, 2H), 6.79 (d, J=8.0 Hz, 1H), 4.96-4.82 (m, 2H), 3.70-3.58 (m, 2H), 3.54-3.44 (m, 3H), 3.31 (s, 3H), 1.98 (s, 3H), 1.36 (d, J=6.9 Hz, 3H).

### Example 11: Design of Linker Sites for CNOT7 Binders

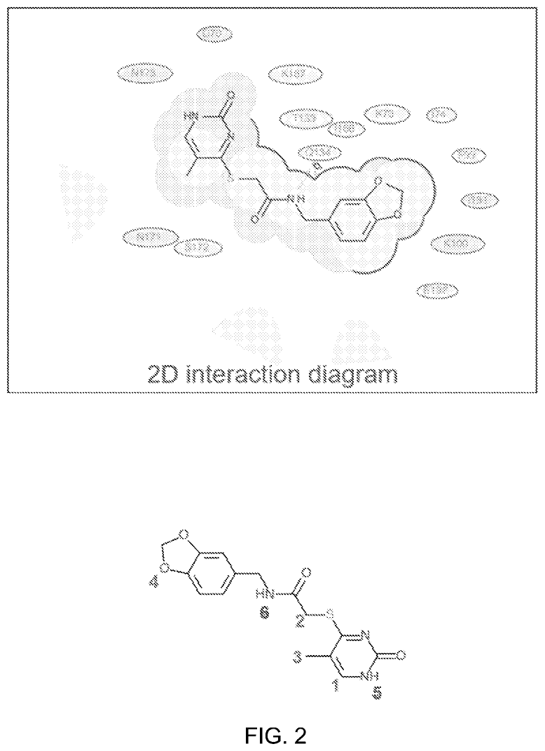

Various CNOT7 binding compounds were modeled for binding into an allosteric pocket of CNOT7. FIG. 2 shows Compound I-1 docked to the CNOT7 protein. Exit vectors that allow for the attachment of a linker to create bifunctional molecules are shown.

### Example 12: Evaluating of Binding of Compounds to CNOT7 by Surface Plasmon Resonance (SPR) and Isothermal Titration Calorimetry (ITC)

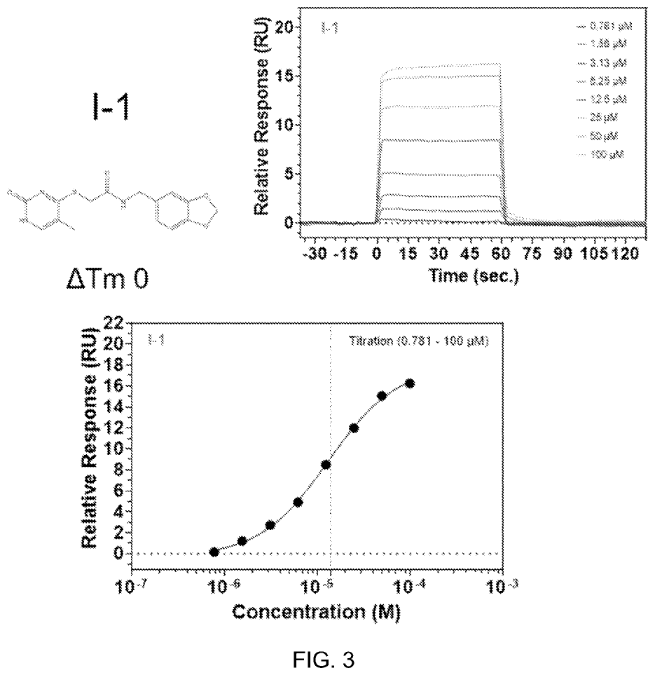

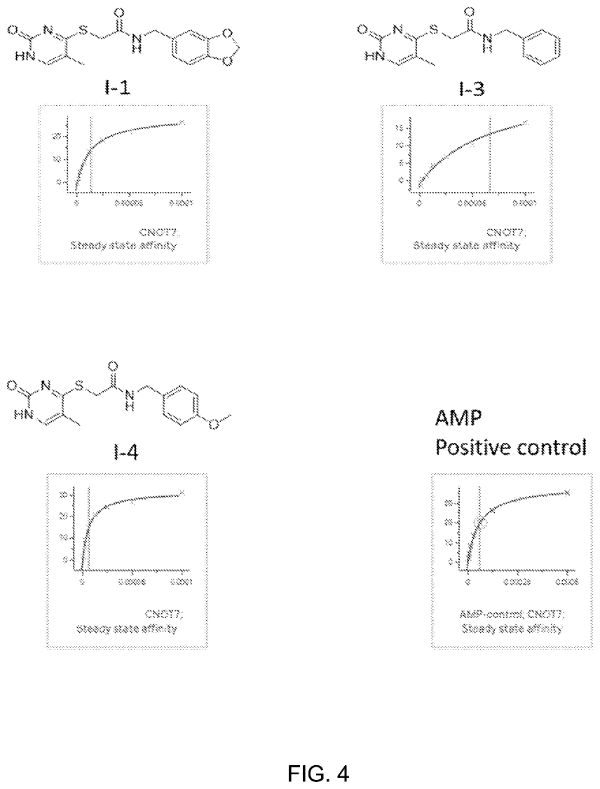

Surface plasmon resonance (SPR) was used to evaluate the binding of compounds to CNOT7. Experiments were performed on a Biacore 8K (Cytiva) with a running buffer consisting of 20 mM HEPES pH7.5, 100 mM KCl, 3 mM MgCl2, 1 mM TCEP, 0.05% tween-20, 2% DMSO. Briefly, biotinylated CNOT7 (Ichor Life Sciences) at a concentration of 70 μg/ml was immobilized to a streptavidin coated chip (Cytiva sensor chip SA) for 1200 sec at a flow rate of 5 μl/min (typical immobilization level 7,500 RU). For compound binding experiments, various concentrations of analyte were flowed over the chip with 120 sec association phase, 120 sec dissociation phase, at a flow rate of 30 ul/min and collection rate of 10 Hz. Reference channel subtraction and solvent correction were applied to all sensograms prior to data analysis. Sensograms were fit to 1:1 steady state affinity model. FIGS. 3 and 4 show the binding of exemplary compounds and the AMP control (a known CNOT7 active site binder) at various concentrations to CNOT7.

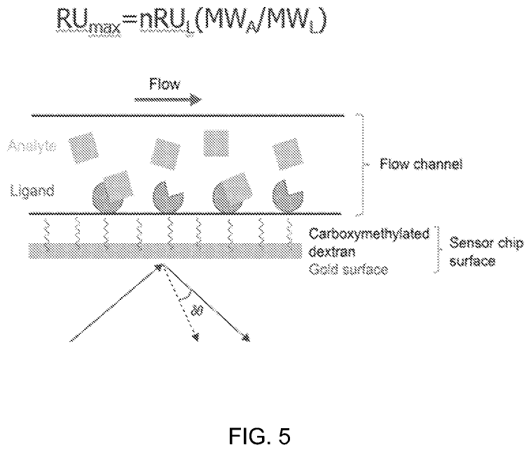

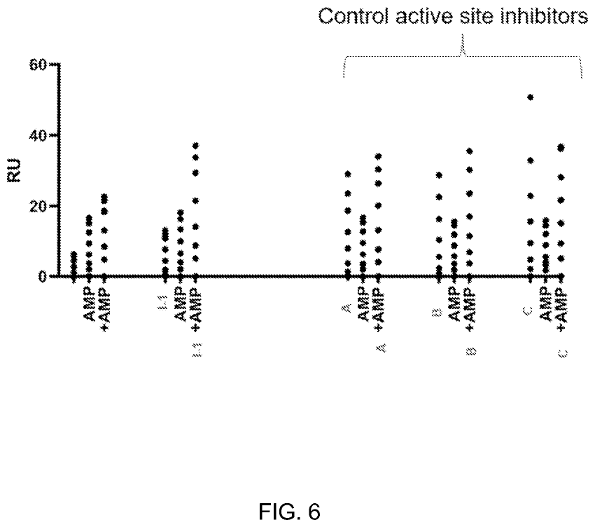

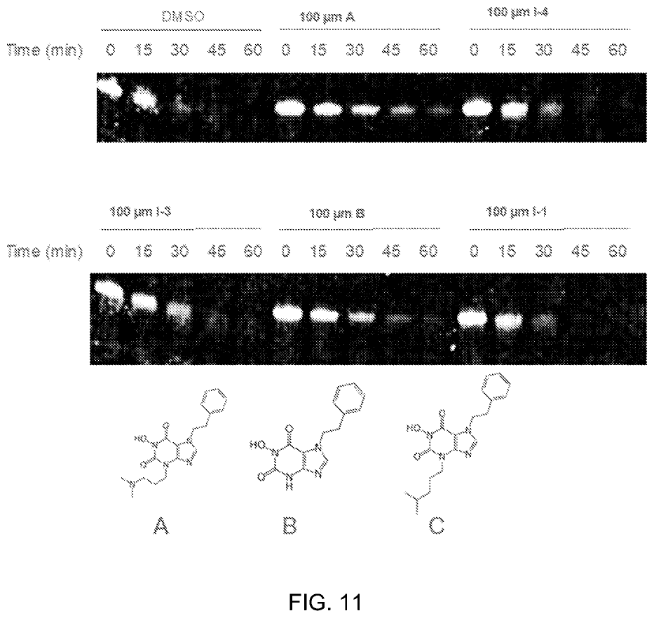

In order to evaluate if the compounds bound to the active site of CNOT7, an AMP competition experiment was performed. FIG. 5 provides a schematic of a surface plasmon resonance (SPR) experiment to interrogate if a CNOT7 binding compound binds in the active site of CNOT7. Briefly, AMP, which is a known binder to the active site, was interrogated at increasing concentrations for binding to CNOT7. As a next step, a CNOT7 binding compound was interrogated at increasing concentrations for binding to CNOT7. As a final step a combination of AMP and CNOT7 binding compound was interrogated by SPR at increasing concentrations for binding to CNOT7. If the signals were additive, AMP and the CNOT7 compounds bind to independent sites on CNOT7. As AMP binds to the active site of CNOT7, an additive signal indicates that the non-AMP compound binds to CNOT7 on a site other than the active site. In contrast, if the signals are not additive, this would indicate that AMP and CNOT7 are not competing for the same site. Table 4 shows the dissociation constant (K_{D}) for some compounds described herein. FIG. 6 shows that compound I-1 have a signal that is additive to AMP, indicating that compound I-1 binds to a location on CNOT7 other than the active site. As a control, literature compounds that are active site binders, compounds A, B and C, are not additive when combined with AMP. This shows that compounds A, B and C are competing for binding to the active site with AMP. The structure of compounds A, B and C is provided in FIG. 11.

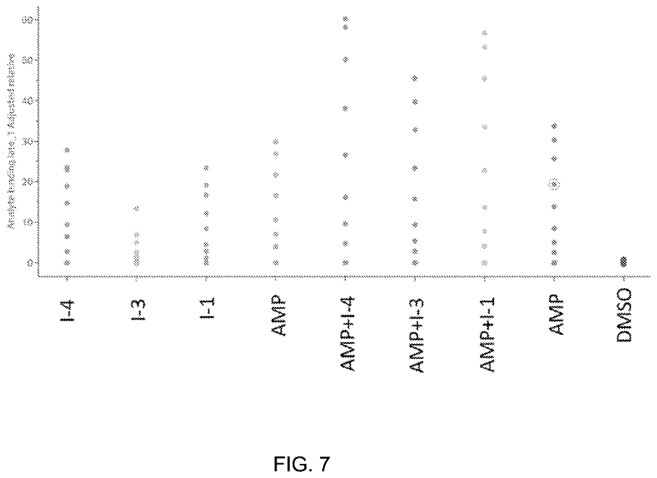

FIG. 7 shows that additional CNOT7 binding compounds disclosed herein, I-3 and I-4 also have a signal that is additive to AMP, indicating that these compounds bind to a location on CNOT7 other than the active site.

Isothermal titration calorimetry (ITC) experiments were performed to evaluate the binding of compounds to CNOT7. All experiments were performed at 25° C. on a Malvern PEAQ-ITC instrument (2bind molecular interactions). Prior to experiments, biotinylated CNOT7 (Ichor Life Sciences) was centrifuged at 18,000 rcf for 30 min at 4 deg followed by buffer exchange via Amicon 10K into 10 mM HEPES pH7.5, 200 mM NaCl, 1 mM TCEP, 1 mM MgCl₂. ITC experiments were performed using 12 consecutive injections of 50 uM ligand (3 μl per injection) into target cell containing 10 μM biotinylated CNOT7 in 10 mM HEPES pH7.5, 200 mM NaCl, 1 mM TCEP, 1 mM MgCl2, 1% DMSO (stirring 750 rpm). Raw heat plots were integrated and data analyzed using PEAQ-ITC Analysis software. DLS (dynamic light scattering) and DSF (differential scanning fluorimetry) were performed on the protein before and after ITC to confirm protein quality over the course of the experiment.

The symbol “***” indicates a K_{D} less than or equal to 1 μM. The symbol “**” indicates a K_{D} in the range of greater than 1 μM to 25 μM. The symbol “*” indicates a K_{D} greater than 25 μM.

TABLE 4

| Compound | |
|---|---|
| Number | K_{D} |
| I-1 | ** |
| I-2 | * |
| I-3 | * |
| I-4 | ** |
| I-5 | * |
| I-6 | * |

### Example 13: Results for Binding of Additional Compounds to CNOT7 by Surface Plasmon Resonance (SPR)

Surface plasmon resonance (SPR) was used to evaluate the binding of compounds to CNOT7 as shown in Example 12 above. Table 5 shows the dissociation constant (K_{D}) for some compounds described herein. The symbol “****” indicates a K_{D} less than or equal to 100 nM. The symbol “***” indicates a K_{D} in the range of greater than 100 nM to 1 μM. The symbol “**” indicates a K_{D} in the range of greater than 1 μM to 25 μM. The symbol “*” indicates a K_{D} greater than 25 μM.

TABLE 5

| Compound | |
|---|---|
| Number | K_{D} |
| I-168 | ** |
| I-169 | ** |
| I-170 | ** |
| I-26 | * |
| I-27 | ** |
| I-28 | ** |
| I-31 | * |
| I-32 | * |
| I-34 | ** |
| I-42 | ** |
| I-43 | * |
| I-44 | ** |
| I-46 | ** |
| I-48 | ** |
| I-50 | ** |
| I-54 | * |
| I-55 | * |
| I-52 | * |
| I-56 | ** |
| I-57 | ** |
| I-51 | * |
| I-197 | ** |
| I-198 | ** |
| I-205 | * |
| I-217 | ** |
| I-219 | ** |
| I-220 | ** |
| I-221 | ** |
| I-226 | ** |
| I-228 | ** |
| I-230 | * |
| I-238 | * |
| I-242 | * |
| I-243 | *** |
| I-266 | ** |
| I-267 | ** |
| I-165 | ** |
| I-166 | ** |
| I-167 | ** |
| I-191 | *** |
| I-192 | ** |
| I-193 | ** |
| I-218 | ** |
| I-225 | * |
| I-239 | * |
| I-240 | * |
| I-247 | ** |
| I-249 | ** |
| I-252 | * |
| I-254 | * |
| I-277 | ** |
| I-287 | * |
| I-294 | ** |
| I-306 | ** |
| I-307 | *** |
| I-314 | ** |

### Example 14: Results for Binding of Additional Compounds to CNOT7 by Surface Plasmon Resonance (SPR)

Surface plasmon resonance (SPR) was used to evaluate the binding of compounds to CNOT7 as shown in Example 6 above. Table 6 shows the dissociation constant (K_{D}) for some compounds described herein. The symbol “****” indicates a K_{D} less than or equal to 100 nM. The symbol “***” indicates a K_{D} in the range of greater than 100 nM to 1 μM. The symbol “**” indicates a K_{D} in the range of greater than 1 μM to 25 μM. The symbol “*” indicates a K_{D} greater than 25 μM.

TABLE 6

| Compound | |
|---|---|
| Number | K_{D} |
| I-310 | *** |
| I-248 | *** |
| I-244 | ** |
| I-312 | ** |
| I-281 | ** |
| I-311 | ** |
| I-45 | ** |
| I-190 | ** |
| I-313 | ** |
| I-172 | ** |
| I-250 | ** |
| I-229 | ** |
| I-203 | ** |
| I-323 | ** |
| I-322 | ** |
| I-324 | ** |
| I-326 | ** |
| I-325 | ** |
| I-261 | ** |
| I-327 | ** |
| I-328 | ** |
| I-173 | ** |
| I-329 | ** |
| I-199 | *** |
| I-330 | *** |
| I-58 | * |

### Example 15: Results for Binding of Additional Compounds to CNOT7 by Surface Plasmon Resonance (SPR)

Surface plasmon resonance (SPR) was used to evaluate the binding of compounds to CNOT7 as shown in Example 6 above. Table 7 shows the dissociation constant (K_{D}) for some compounds described herein. The symbol “****” indicates a K_{D} less than or equal to 100 nM. The symbol “***” indicates a K_{D} in the range of greater than 100 nM to 1 μM. The symbol “**” indicates a K_{D} in the range of greater than 1 μM to 25 μM. The symbol “*” indicates a K_{D} greater than 25 μM.

TABLE 7

| Compound | |
|---|---|
| Number | K_{D} |
| I-326 | ** |
| I-342 | ** |
| I-349 | ** |
| I-324 | *** |
| I-330 | ** |
| I-367 | ** |
| I-368 | ** |
| I-329 | ** |
| I-370 | * |
| I-371 | * |
| I-372 | ** |
| I-373 | * |
| I-378 | ** |
| I-384 | * |
| I-392 | ** |
| I-395 | ** |
| I-397 | ** |
| I-400 | * |
| I-401 | * |
| I-402 | ** |
| I-404 | ** |
| I-405 | ** |
| I-407 | ** |
| I-408 | ** |
| I-409 | * |
| I-410 | * |
| I-411 | ** |
| I-412 | ** |
| I-415 | * |
| I-416 | ** |
| I-421 | * |
| I-426 | ** |
| I-428 | ** |
| I-430 | * |
| I-431 | ** |
| I-432 | ** |
| I-433 | * |
| I-435 | * |
| I-440 | * |
| I-332 | ** |
| I-328 | ** |
| I-331 | ** |
| I-332 | ** |
| I-325 | ** |
| I-323 | ** |
| I-333 | ** |
| I-334 | ** |
| I-338 | * |
| I-339 | * |
| I-327 | ** |
| I-340 | * |
| I-443 | ** |
| I-444 | ** |
| I-445 | ** |
| I-451 | * |
| I-457 | ** |
| I-458 | ** |
| I-459 | * |
| I-464 | ** |
| I-465 | ** |
| I-468 | ** |
| I-469 | ** |
| I-470 | ** |
| I-471 | ** |
| I-472 | ** |
| I-473 | ** |
| I-474 | ** |
| I-475 | ** |
| I-476 | ** |
| I-477 | ** |
| I-478 | ** |
| I-479 | * |
| I-480 | * |
| I-482 | ** |
| I-484 | ** |
| I-485 | * |
| I-487 | * |
| I-488 | ** |
| I-489 | ** |
| I-490 | ** |
| I-491 | * |
| I-492 | * |
| I-493 | ** |
| I-495 | * |
| I-496 | * |
| I-497 | * |
| I-498 | * |
| I-499 | ** |
| I-500 | ** |
| I-501 | ** |
| I-502 | ** |

### Example 16: Evaluating the Degradation of RNA by CNOT7

An assay was established that showed the ability of CNOT7 to degrade RNA, and the acceleration of such degradation by bifunctional molecules. Fluorescently labeled RNA substrates were synthesized (Integrated DNA Technologies) with the following sequences:

(SEQ ID NO: 1)
5′ FAM-AGGGAAGGGCUGGGAUGGCAGUAGACUUGGCUUU
CCCAUUACUCUUUUCUAAAAAAAAAA
(SEQ ID NO: 2)
5′FAM-AGGGAAGGGCUGGGAUGGCAGUAGACUUGGCUUUC
CCAUUACUCUUUUCU

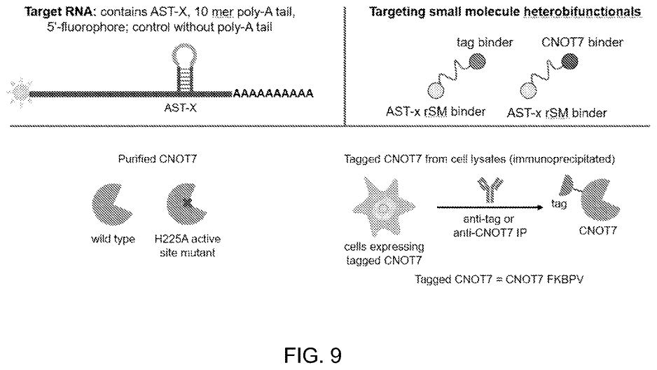

SEQ ID NO:2 corresponds to AST-X, while SEQ ID NO:1 corresponds to SEQ ID NO:2 with a poly-A tail. See also the top left panel in FIG. 9.

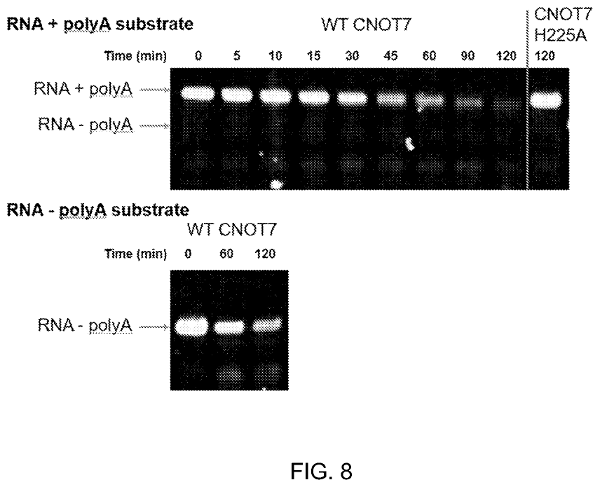

Substrates (500 nM) were combined with wild-type or catalytic mutant (H225A) CNOT7 recombinant enzyme (100 nM) in the following buffer conditions: 50 mM HEPES pH 7.3, 10 mM KCl, 45 mM NaCl, 2 mM MgCl2, 0.1 mM TCEP, 0.5% glycerol. The catalytic mutant of CNOT7 has an H225A mutation in its active site and is not able to degrade RNA. The reactions were incubated at 37° C., with specified timepoints removed from the reaction and combined with 2× formamide RNA loading buffer with 10 mM EDTA before boiling for 3 minutes to quench the reaction. Samples were run on a pre-warmed 15% TBE-Urea polyacrylamide gel at 180V for 90 minutes and then imaged using Cy2 settings on the Azure Biosystems 600 Imaging System. As shown in FIG. 8, deadenylation/degradation of the substrate band over the course of 2 hours was observed, dependent on the catalytic activity of CNOT7, and specific to the adenylated substrate RNA.

### Example 17: Evaluating the Degradation of RNA by CNOT7 Binding Compounds in Biochemical Assays

Biochemical reporter systems were generated to evaluate the ability of CNOT7 binding compounds to accelerate the degradation of RNA by CNOT7. FIG. 9 shows a schematic of biochemical assays used to evaluate the ability of CNOT7 binders to increase the degradation of RNA. Generation of the target RNA was described in the previous experiment. The top right-hand panel shows bifunctional molecules that can increase the degradation of RNA by CNOT7. The bifunctional molecules consist of Compound 1-4 and the following rSM:

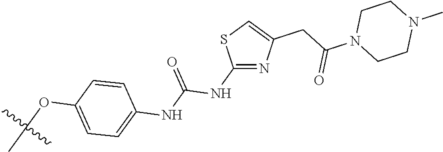 [SMILES: *Oc1ccc(NC(=O)Nc2nc(CC(=O)N3CCN(C)CC3)cs2)cc1]

which binds to the RNA sequence of AST-X, or the rSM linked to a tag that binds CNOT7-FKBPV. Specifically, the tag is compound AP1867 (CAS NO.: 195514-23-9), a known binder of FKBPV, resulting in the rSM-AP1867tag. The CNOT7-FKBPV is obtained by immunoprecipitation of cell engineered to express CNOT-FKBPV. Specifically, H1299 cells were transfected with plasmid expressing HA-tagged CNOT7-FKBP12(F36V) fusion protein. After 24 hours, cells were pelleted and lysed in the following lysis buffer: 20 mM HEPES pH 7.3, 0.2 mM EGTA, 10% glycerol, 0.1 mM DTT with cOmplete EDTA-free protease inhibitors (Millipore). After freeze-thawing, NaCl was added to a concentration of 400 mM and lysates were pelleted to clear cellular debris. Cleared lysate was adjusted to buffer conditions containing 0.1% NP-40 and 150 mM NaCl before being applied to Pierce Anti-HA magnetic beads (ThermoFisher) at 4° C. for 2-20 hours with end-over-end rotation. After washing, bound protein was competitively eluted with 2 mg/mL HA peptide (ThermoFisher) in activity assay buffer at 4° C. for 2-20 hours. It should be appreciated that this immunoprecipitation protocol will result in tagged CNOT7 that will likely have other components of the CCR4-NOT complex bound as well.

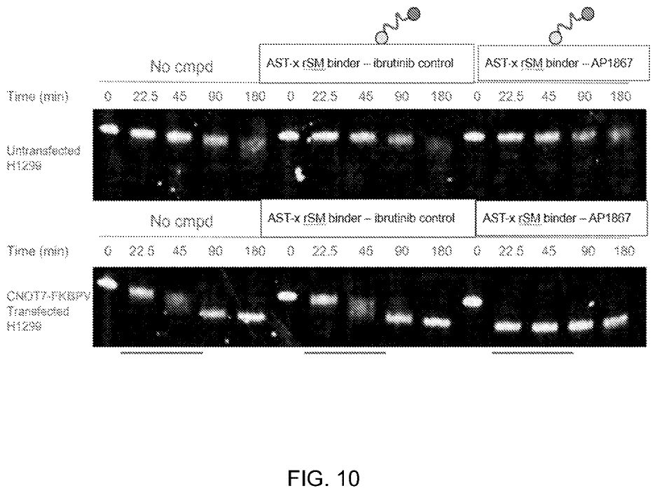

Heterobifunctional molecules were generated by conjugating Compound I-4 to the rSM, which binds RNA with an AST-X sequence, conjugating the rSM to AP1867 (a small molecule binder of FKBP12(F36V), resulting in rSM-AP1867tag, or conjugating the rSM to a non-covalent ibrutinib, which does not bind FKBP12 and acts a control. Fluorescently labeled RNA substrates were refolded in the presence of Mg²⁺ and incubated with equimolar heterobifunctional molecules before enzymatic reaction was initiated with the elution fraction (containing CNOT7-FKBP12(F36V) fusion protein) from the immunoprecipitation protocol above. The reactions were incubated at 37° C., with specified timepoints removed from the reaction and combined with 2× formamide RNA loading buffer with 10 mM EDTA before boiling for 3 minutes to quench the reaction. Samples were run on a pre-warmed 15% TBE-Urea polyacrylamide gel at 180V for 90 minutes and then imaged using Cy2 settings on the Azure Biosystems 600 Imaging System. As shown in FIG. 10, a 4-8-fold increased deadenylation rate of our RNA substrate was observed when pre-incubated with the on-target heterobifunctional molecule (right side of gels) compared with the off-target heterobifunctional (middle of gels) or no compound control (left side of gels). This shows that heterobifunctional molecules that bind RNA with one functionality and CNOT7 or fusion proteins thereof with the other functionality can facilitate the degradation of RNA by CNOT7 or potentially degradation by other components of the CCR4-NOT complex that were obtained in the immunoprecipitation. It should be noted that the CCR4-NOT complex includes proteins other than CNOT7, for instance CNOT6, that have deadenylation and/or exonuclease activity.

As shown previously, e.g., By SPR, the CNOT7 binding compounds disclosed herein do not bind the active site of CNOT7. The compounds therefore do not abrogate the ability of CNOT7 to degrade RNA. These findings were confirmed in the biochemical assay described above. As shown in FIG. 11, CNOT7 binders do not abrogate CNOT7 function. Compounds I-1, 1-3 and 1-4 when added to the CNOT7 and RNA mixture did not interfere with the ability of CNOT7 to degrade RNA. In contrast, literature compounds that are active site binders, compounds A, B and C, structure depicted on the right-hand side, do interfere with the ability of CNOT7 to degrade RNA (See center panel of gel).

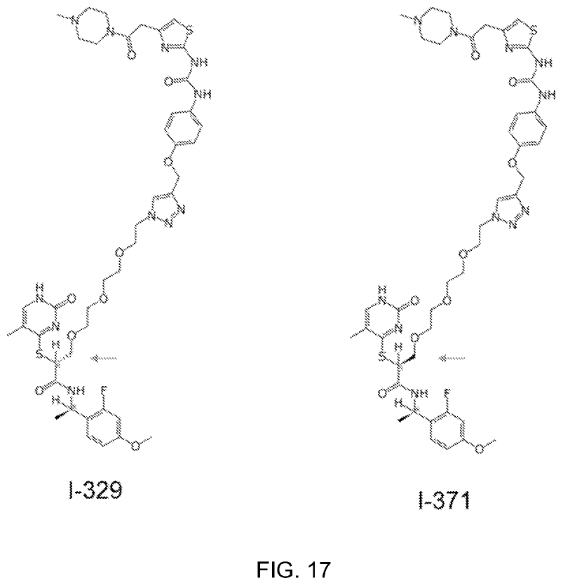

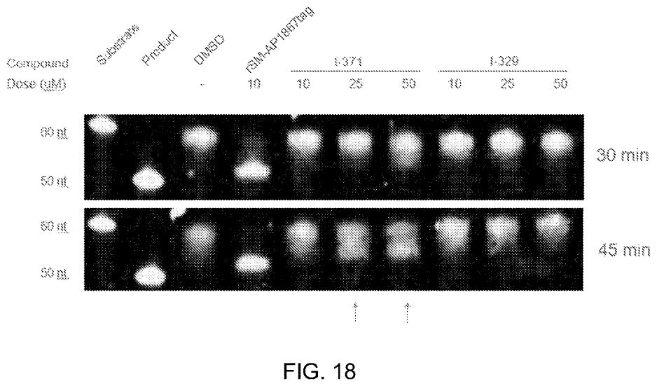

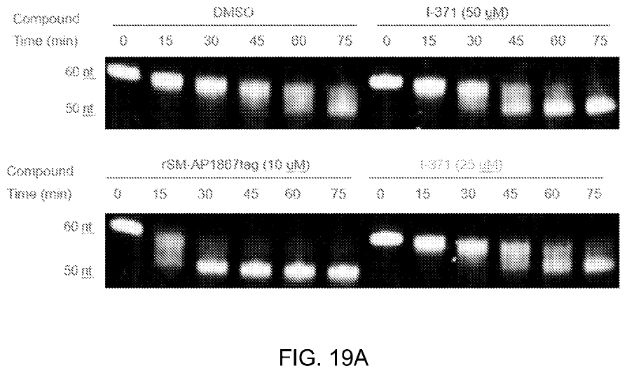

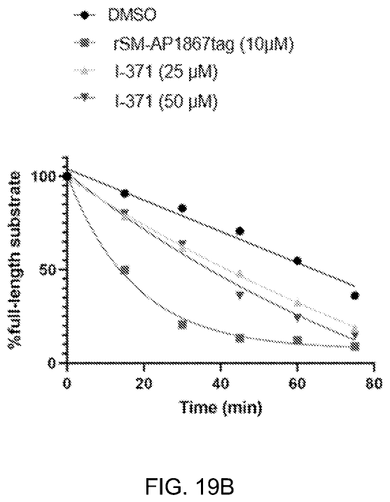

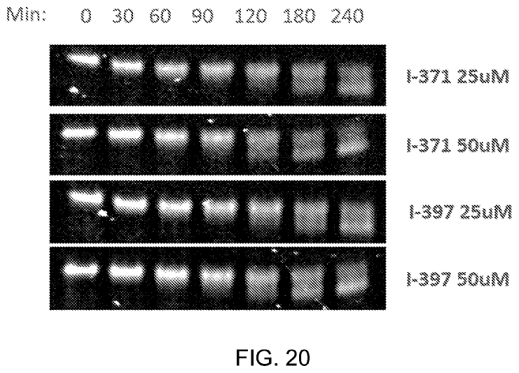

Heterobifunctional molecules were generated by conjugating the rSM depicted above, which binds RNA with an AST-X sequence, to AP1867 (a small molecule binder of FKBP12(F36V)), resulting in rSM-AP1867tag. In addition, I-243 was conjugated to the rSM resulting in compounds 1-329 and I-371, depicted in FIG. 17. Fluorescently labeled RNA substrates were refolded in the presence of Mg²⁺ and incubated 10, 25, or 50 μM doses of heterobifunctional molecules for 30 minutes at room temperature before the enzymatic reaction was initiated with the elution fraction (containing CNOT7-FKBP12(F36V) fusion protein) from the immunoprecipitation protocol above. The reactions were incubated at 37° C., with specified timepoints removed from the reaction and combined with 2× formamide RNA loading buffer with 10 mM EDTA before boiling for 3 minutes to quench the reaction. Samples were run on a pre-warmed 15% TBE-Urea polyacrylamide gel at 180V for 120 minutes and then imaged using Cy2 settings on the Azure Biosystems 600 Imaging System (See FIGS. 18, 19A and 19B). In addition, for 1-371 and the control rSM-AP1867tag, the disappearance of the full-length substrate band was quantified using ImageJ and plotted over time (FIG. 19B). As shown in FIG. 19A, a ~1.5-fold increased deadenylation rate of the RNA substrate was observed when pre-incubated with either a 25 uM or 50 uM dose of the CNOT7-binding heterobifunctional molecule 1-371 (right side of gels) compared with DMSO (left side of top gel). This shows that heterobifunctional molecules that bind RNA with one functionality and CNOT7 with the other functionality can facilitate the degradation of RNA by CNOT7.

### Example 18: Results for Degradation of RNA by CNOT7 Binding Heterobifunctional Compounds in Biochemical Assays

Results for degradation of RNA by additional CNOT7 binding heterobifunctional compounds as described in the assay in Example 17 are shown in Table 8 below.

The symbol “+” indicates an activity observed as compared to DMSO control. The symbol “−” indicates no activity observed as compared to DMSO control.

TABLE 8

| Compound Number | Activity by gel |
|---|---|
| I-329 | − |
| I-371 | + |
| I-397 | + |
| I-398 | − |
| I-372 | + |
| I-373 | + |
| I-382 | + |
| I-454 | + |
| I-533 | − |
| I-396 | − |
| I-395 | + (weak) |
| I-469 | − |
| I-468 | + |
| I-498 | + (weak) |
| I-497 | + (weak) |
| I-496 | + |
| I-495 | − |
| I-465 | + |
| I-464 | − |
| I-471 | − |
| I-470 | − |
| I-503 | − |
| I-504 | − |
| I-506 | − |
| I-505 | − |
| I-514 | − |
| I-515 | − |
| I-509 | − |
| I-510 | − |
| I-477 | − |
| I-478 | + |
| I-473 | − |
| I-474 | − |
| I-475 | − |
| I-476 | − |
| I-384 | |
| I-459 | − |
| I-339 | + |
| I-338 | − |
| I-451 | − |

### Example 19: Evaluating the Degradation of RNA by CNOT7 Binding Compounds in Cellular Assays

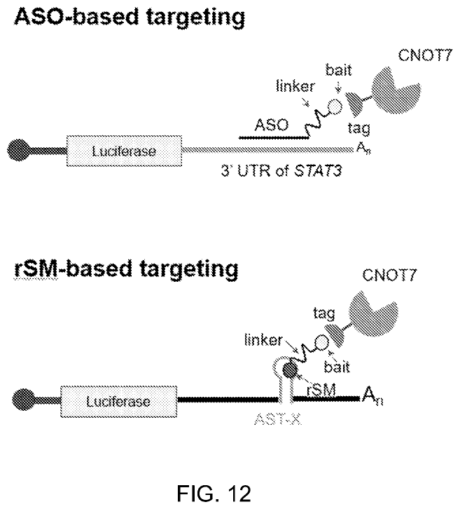

Reporter constructs for cellular assays are depicted in FIG. 12. A luciferase reporter construct was designed to the 3′ UTR of STAT3 fused to Renilla luciferase, which contains the PEST and CP1 destabilizing domains. A second luciferase reporter construct was designed using the 3′UTR of HBB fused to Firefly luciferase, which contains the PEST and CP1 destabilizing domains. The reporter design for both constructs was previously described in Luo E C, et al. Nat Struct Mol Biol. 2020; 27(10):989-1000.

An antisense oligo was conjugated to the bait molecule AP1867, which binds FKPB12 via click-chemistry to generate a heterobifunctional molecule (ASO-bait). The bait molecule was chemically modified to contain an azide to allow for click chemistry. The oligonucleotide sequence is chemically modified to contain locked nucleic acids at alternating positions, phosphorothioate linkages at every position, and addition of a 5′ DBCO-TEG. The oligonucleotide portion of the ASO-bait was designed to bind to a sequence in the 3′UTR of STAT3 for the targeting ASO-bait (SEQ ID NO:3) or is a scrambled control sequence for the non-targeting ASO-bait. (SEQ ID NO:4).

| | Sequence (Asterisk | |
|---|---|---|
| | indicates | |
| | phosphorothioate | |
| | linkage; {N} | |
| | indicates a locked | Length |
| | nucleic acid) | (nt) |
| Targeting ASO | G*{A*}A*{G*}T*{A*}C* | 14 |
| (SEQ ID NO: 3) | {A*}C*{A*}T*{T*}G*{G} | |
| Non-targeting | G*{A*}C*{T*}G*{T*}A* | 15 |
| ASO | {G*}G*{T*}T*{T*}A* | |
| (SEQ ID NO: 4) | {T*}C | |

A similar reporter was designed by incorporating the RNA sequence AST-X instead of the 3′UTR of STAT3.

The reporter systems were used together with plasmids that express CNOT7 tagged fusion proteins. Specifically, plasmids were designed fusing an FKBP12 variant (FKBP12^{F36V}; See, e.g., Nabet et al Nat Chem Biol. 2018; 14(5):431-441), to a CNOT7 or GFP sequence to generate a CNOT7 fusion constructs. The FKBP12 variant selectively recognizes the ligand, AP1867, which serves as the bait in this system. The GFP fusion plasmids serves as a negative control for CNOT7 activity.

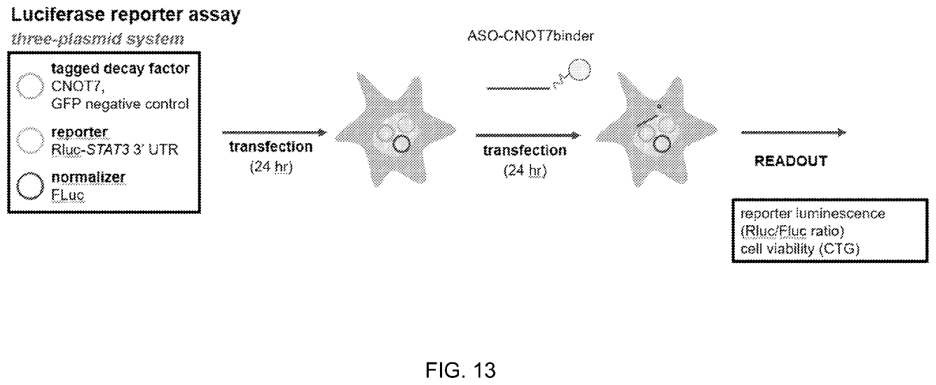

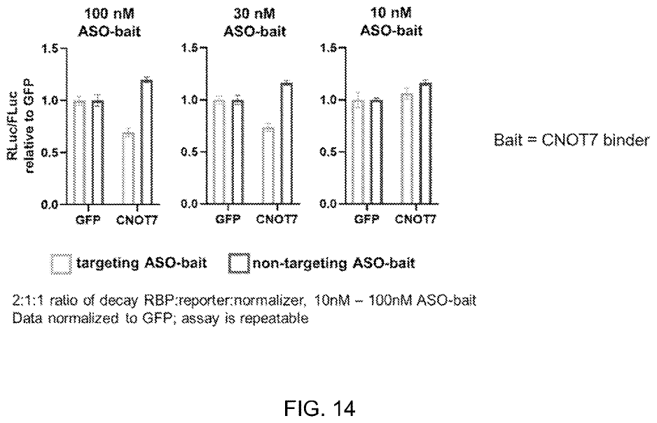

A model system to interrogate the ability of a bifunctional RNA binder—CNOT7 binder is depicted in FIG. 13. A parallel approach utilizing a luciferase readout together with mRNA expression analysis was employed to assess the bifunctional molecule's capability to recruit CNOT7 to target mRNA and mediate mRNA degradation. H1299 cells were seeded in a 96-well plate the day before transfection. On the next day, the cells were transfected with a 2:1:1 ratio of CNOT7, Renilla STAT3 3′UTR reporter, and the Firefly HBB normalizer, respectively, and incubated for 24 hours. On the following day, cells were transfected with 10 nM, 30 nM, and 100 nM of the targeting or non-targeting ASO-bait molecules and incubated for 24 hours. Dual luciferase assay (Promega) for Renilla and Firefly was performed after the ASO-bait incubation. A parallel plate was assessed for cell viability using CellTiter Glo (Promega). Data was analyzed by normalizing reporter signal relative to normalizer signal, and this ratio was then normalized to GFP. All transfections were performed using Lipofectamine 3000 (Invitrogen). The data, depicted in FIG. 14 show that a reduction in reporter signal is observed in the presence of the targeting ASO-bait with CNOT7 relative to conditions with the non-targeting ASO-bait. Additionally, the reduction in reporter signal is sensitive to the amount of ASO-bait present. Thus, the bifunctional molecule is capable of recruiting CNOT7 intracellularly resulting in an increase in the degradation of a target RNA.

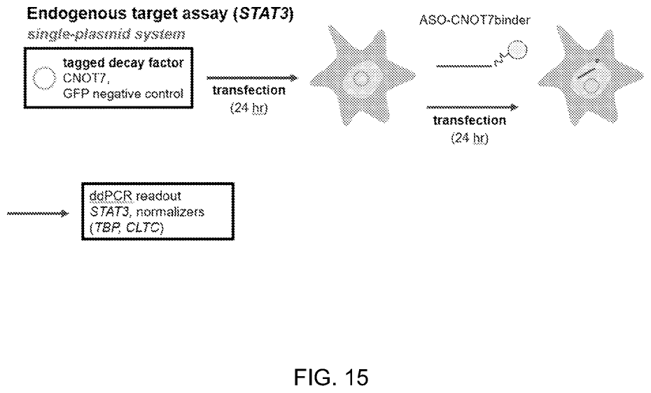

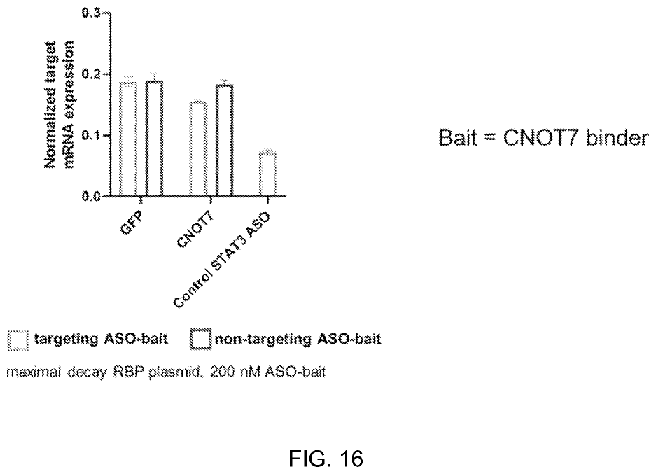

The approach described above was modified to shows that the bifunctional molecule can degrade endogenous cellular RNA (STAT3), as shown in FIG. 15. For the endogenous mRNA expression assay, H1299 cells were seeded in a 12-well plate the day before transfection. On the following day, the cells were transfected with the plasmid expressing tagged CNOT7 and incubated for 24 hours. The following day, cells were transfected with 200 nM of the targeting or non-targeting ASO-bait molecules and incubated for 24 hours. In addition, cells were also transfected with a known STAT3 silencing ASO as a positive control for STAT3 mRNA knockdown. Samples were directly lysed in-well for RNA purification using the Promega Maxwell Automated RNA Extractor, followed by single-strand cDNA synthesis. The resulting cDNA was serially diluted at 1:100, which was used in the ddPCR reaction with primers designed to STAT3 and housekeeping genes (CLTC and TBP), the sequences of which are shown below. Data were analyzed by normalizing STAT3 relative to the geomean of TBP and CLTC. All transfections were performed using Lipofectamine 3000 (Invitrogen). As depicted in FIG. 16, the data show a that a reduction in STAT3 expression is observed in the presence of the targeting ASO-bait with CNOT7 relative to conditions with the non-targeting ASO-bait and relative to the GFP control. Thus, the bifunctional molecule is capable of accelerating the degradation of endogenously expressed RNA.

Primer sequences were designed to STAT3, CLTC, and TBP as listed in the table below.

| | | |
|---|---|---|
| SEQ ID | TGGCCTGTGTT | CLTC forward |
| NO: 5 | GCTTATGAA | |
| SEQ ID | CCAATGTGATC | CLTC_internal. FAM MGB- |
| NO: 6 | TGGAACT | NFQ probe |
| SEQ ID | CCAATGTGATC | CLTC_internal. VIC MGB-NFQ |
| NO: 7 | TGGAACT | probe |
| SEQ ID | CGTACCAGGTA | CLTC_reverse |
| NO: 8 | GCGAGAAAG | |
| SEQ ID | TGATCTTTGCA | TBP_forward |
| NO: 9 | GTGACCCAG | |
| SEQ ID | CTTGGCGTGTG | TBP_internal. FAM MGB-NFQ |
| NO: 10 | AAGAT | probe |
| SEQ ID | CTTGGCGTGTG | TBP_internal. VIC MGB-NFQ |
| NO: 11 | AAGAT | probe |
| SEQ ID | CTAGAGCATCT | TBP_reverse |
| NO: 12 | CCAGCACAC | |
| SEQ ID | GAAACTCTTGG | STAT3 ex18-19 forward |
| NO: 13 | GACCTGGTGTG | |
| | AA | |
| SEQ ID | TTATTCAGGGT | STAT3 ex18-19 internal. |
| NO: 14 | GTCAGATCA | FAM MGB-NFQ probe. |
| SEQ ID | CTTGCCAGCCA | STAT3 ex18-19 reverse |
| NO: 15 | TGTTTTCTTTG | |
| | CAA | |

### Example 20: CNOT7 Inhibition Assays

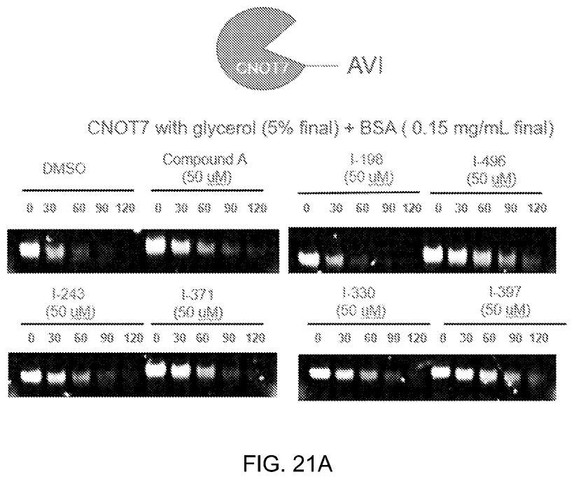

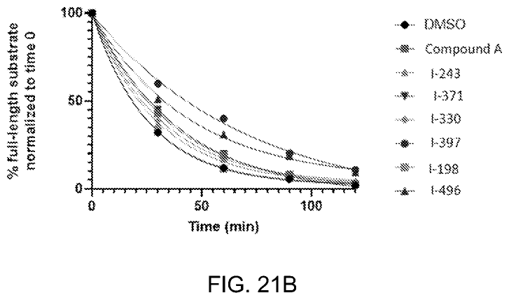

Recombinant CNOT7 (with a C-terminal Avi tag) was produced in E. coli and purified to high purity. For this biochemical assay, deadenylation reactions are carried out by refolding 2 microM fluorescently labeled RNA substrate (5′ 6-FAM-AGGGAAGGGCUGGGAUGGCAGUAGACUUGGCUUUCCCAUUACUCUUUUCUAAAA AAAAAA (SEQ ID NO:1) in the presence of 100 mM KCl and 3 mM MgCl₂ at 37 C for 30 minutes and then pre-incubating the RNA with 500 microM compound (inhibitor, heterobifunctional, or the CNOT7 binding moiety of the heterobifunctional) at room temperature for 30 minutes (in the presence of 0.05% Tween-20) before initiating the deadenylation reaction with the addition of the recombinant CNOT7 (with Avi-tag). In the reactions, the final concentration of RNA was 200 nM, the final concentration of the compounds was 50 μM, and the final concentration of CNOT7 was 100 nM. Glycerol and BSA were included in the reactions to aid in protein stability and activity (final concentrations of 5% and 0.15 mg/mL, respectively). Timepoints were removed from the reaction and quenched by adding formamide loading dye including 10 mM EDTA. Samples were run on 15% TBE-Urea gels at 180V for 2-2.5 hours and imaged using an Azure imager with FAM detection settings. Bands were quantified using ImageJ and % full length substrate over time was normalized to band intensity at time zero. The data are shown in FIGS. 21A and 21B.

A specific compound was qualified as a CNOT7 inhibitor, i.e. a suppressor of CNOT7 activity, if a decrease in the rate of deadenylation was observed in the presence of a compound as compared to DMSO control in which no compound was added. Two of the three CNOT7 binding moieties (i.e., baits) that were evaluated (I-243, and 1-330) slightly inhibited CNOT7 activity, while the CNOT7 binding moiety 1-198 did not inhibit CNOT7 activity. This was to be expected as the CNOT7 binding moieties are not expected to bind in the active site or interfere with the ability of CNOT7 to deadenylate RNA. The heterobifunctionals that correspond to the three CNOT7 binding moieties, in which the CNOT7 binding moiety was linked to an rSM (I-371, I-496 and I-397) were able to inhibit some of the CNOT7 activity. We speculate that the inhibition is caused by specific interaction of the heterobifunctional compounds with the recombinant Avi-tagged CNOT7. As expected, the known CNOT7 active site binder Compound A (See FIG. 11) was also able to inhibit some of the CNOT7 activity.

### Example 21: CNOT7:CNOT1 Recombinant Assay

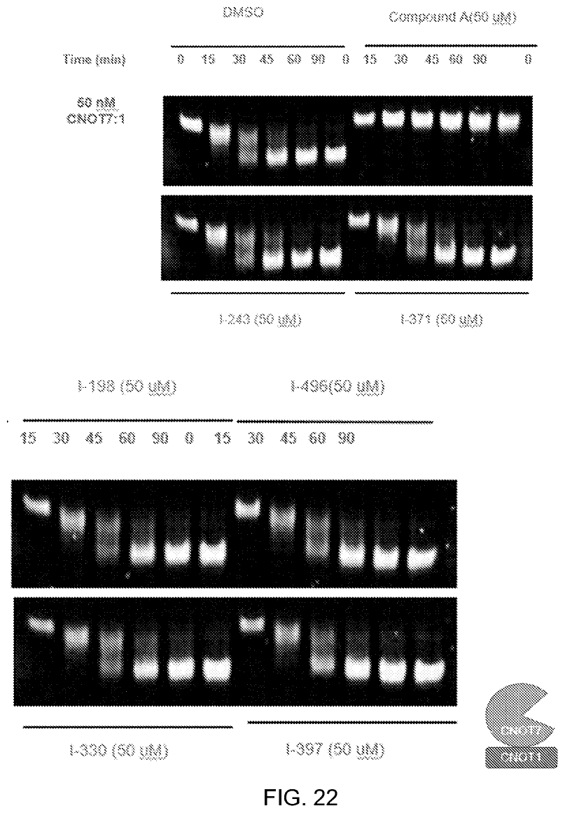

Recombinant CNOT7:CNOT1 (MIF4G domain: aa 1093-1317) complex was produced in E. coli including an 8×His affinity tag, which was cleaved prior to use. The resulting untagged complex was purified to high purity (Helix Biosciences). For this biochemical assay, deadenylation reactions are carried out by refolding 2 microM fluorescently labeled RNA substrate (5′ 6-FAM-AGGGAAGGGCUGGGAUGGCAGUAGACUUGGCUUUCCCAUUACUCUUUUCUAAAA AAAAAA (SEQ ID NO:1)) in the presence of 100 mM KCl and 3 mM MgCl₂ at 37 C for 30 minutes and then pre-incubating the RNA with 500 microM compound (inhibitor, heterobifunctional, or the CNOT7 binding moiety of the heterobifunctional) at room temperature for 30 minutes (in the presence of 0.05% Tween-20) before initiating the deadenylation reaction with the addition of recombinant CNOT7:CNOT1 complex. In the reactions, the final concentration of RNA was 200 microM, the final concentration of the compounds was 50 microM, and the final concentration of CNOT7:CNOT1 was 50 nM. Glycerol and BSA were included in the reactions to aid in protein stability and activity (final concentrations of 5% and 0.15 mg/mL, respectively). Timepoints were removed from the reaction and quenched by adding formamide loading dye including 10 mM EDTA. Samples were run on 15% TBE-Urea gels at 180V for 2-2.5 hours to resolve substrate and product bands and imaged using an Azure imager with FAM detection settings. Bands were quantified using ImageJ and % full length substrate over time was calculated by [Intensity of substrate band]/[Total intensity of full lane of gel]. The data are shown in FIG. 22.

The data show that neither the CNOT7 binding moieties (i.e., baits) that were evaluated (1-243, I-330 and I-198) nor the three CNOT7 heterobifunctionals, in which the CNOT7 binding moiety was linkered to an rSM (1-317, I-496 and ARK I-397) inhibited or accelerated the deadenylation activity of the CNOT7:CNOT1 complex. As expected, the known CNOT7 active site binder Compound A (See FIG. 11) was able to inhibit the CNOT7 activity.

### Example 22: Biochemical AMP-Glo Assay to Measure Deadenylation

The CCR4-NOT complex is generated via a pulldown method. First, H1299 cells are transfected with a plasmid expressing CNOT7 with a C-terminal FKBP(F36V) fusion and an HA affinity tag. 24 hours later, the cells are harvested and lysed via hypotonic lysis. Cleared lysate is applied to equilibrated Anti-HA Magnetic Beads (Pierce) for 24 hours, rotating at 4° C. Beads are then washed three times, and CNOT7 (and associated CCR4-NOT complex members) are eluted via excess synthetic HA peptide into assay buffer. Glycerol is then added to a final concentration of 5%. Immunoblotting and/or mass spectrometry is performed to confirm the presence of CCR4-NOT complex members. Activity assays are performed on each batch to assess overall activity and choose dilution factors and timepoints for subsequent experiments. Triplicate deadenylation reactions are carried out by refolding 8 microM fluorescently labeled RNA substrate (5′ 6-FAM-AGGGAAGGGCUGGGAUGGCAGUAGACUUGGCUUUCCCAUUACUCUUUUCUAAAA AAAAAA) SEQ ID NO:1 in the presence of 100 mM KCl and 3 mM MgCl₂ at 37° C. for 30 minutes and then pre-incubating the RNA with 100 μM bifunctional molecule at room temperature for 30 minutes before initiating the deadenylation reaction with the addition of CCR4-NOT complex. In the reactions, the final concentration of RNA was 0.8 μM and the final concentration of compounds was 10 μM. After 20 minutes at 37° C., reactions are quenched by adding EDTA to a final concentration of 10 mM. AMP detection is performed using the AMP-Glo kit from Promega. In short, 10 μL quenched reactions are mixed with 10 μL AMP-Glo Reagent I, shaken at 500 rpm for 2 min, then centrifuged at 1000 rpm for 1 min. Plate is incubated at room temperature for 1 hour. Then, AMP Detection Solution is prepared by mixing Kinase-Glo and AMP Reagent II at 100:1 proportion, and 20 μL of AMP Detection Solution is added to samples. Again, plates are shaken at 500 rpm for 2 min, then centrifuged at 1000 rpm for 1 min. Plate is incubated at room temperature for 1 hour. Luminescence readings are taken using an Envision plate reader. Raw luminescence values are normalized to DMSO background and values for each compound are reported as fold change over DMSO.

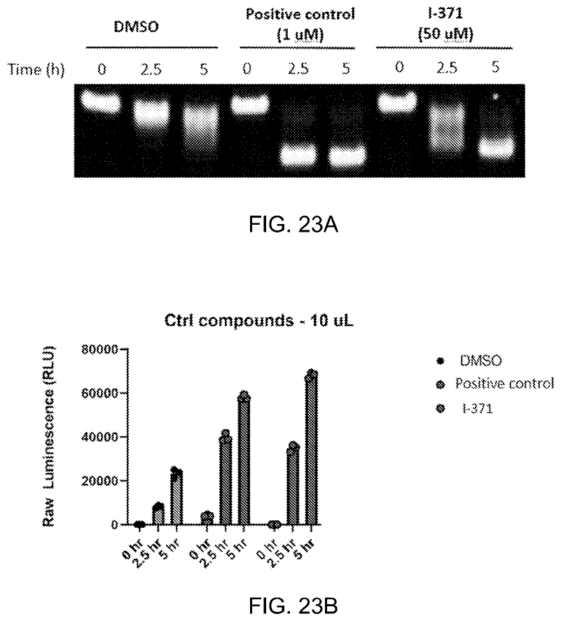

Matched samples were also combined with formamide loading dye and run on a 15% TBE-Urea gel to resolve substrate and deadenylated product bands. Gels were imaged on an Azure imager with FAM detection settings. The data are shown in FIGS. 23A and 23B. The data show that AMP increases over the time course in all conditions and the heterobifunctional compounds, as expected, and that more AMP is generated for the heterobifunctional compounds compared to DMSO background.

### Example 23: Stable CNOT7 Cell Line Assay

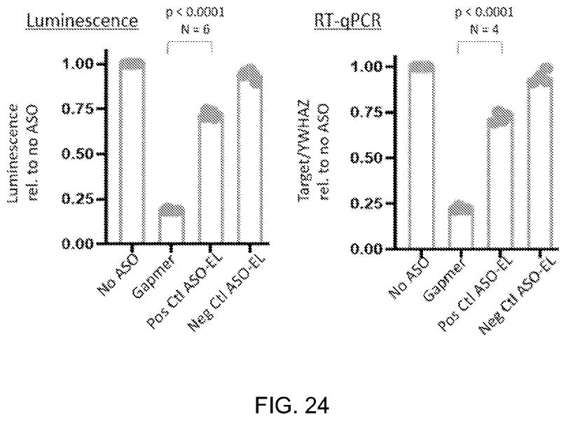

The synthesis of the ASO-bait molecules to a sequence in the 3′UTR of STAT3 for the targeting ASO-bait (SEQ ID NO:3) or its scrambled control sequence for the non-targeting ASO-bait (SEQ ID NO:4) is described in Example 17. An endogenous STAT3 reporter cell line was generated by CRISPR-Cas9 insertion of a HiBiT (Promega) sequence at the native STAT3 stop codon in PC9 cell line, followed by single-cell cloning. A validated clone was then infected with lentivirus generated from plasmids designed by fusing an FKBP12 variant (FKBP12F36V, See, e.g., Nabet et al Nat Chem Biol. 2018; 14(5):431-441), to a CNOT7 sequence as described in Example 11, followed by puromycin selection and western blotting to confirm expression of the CNOT7-FKBP12 fusion variant. The stably transduced pools were maintained with puromycin selection throughout the course of cell culture. A parallel approach utilizing a luciferase readout together with mRNA expression analysis was employed to assess the bifunctional molecule's capability to recruit CNOT7 to target mRNA and mediate mRNA degradation. The CNOT7-FKBP variant stable cell line described above was seeded in a 96-well plate for luciferase readout and 12-well plate for qRT-PCR the day before transfection. On the next day, the cells were transfected with 100 nM of ASO-baits and incubated for 24 hours. The ASO-baits evaluated were the STAT3 targeting ASO-bait (SEQ ID NO:3; this ASO-bait is also referred to as “Pos Ctl; ASO-EL”) and a scrambled control sequence for the non-targeting ASO-bait (SEQ ID NO:4; this ASO-bait is also referred to as “Neg Ctl; ASO-EL”). A positive control transfection was performed in parallel using a STAT3 silencing ASO (100 nM Gapmer; AZD9150) to confirm transfection efficiency and mRNA knockdown. For luciferase readout, NanoGlo HiBiT (Promega) luciferase assay was performed after the ASO-bait incubation. A parallel plate was assessed for cell viability using CellTiter Glo (Promega). Data were analyzed by normalizing reporter signal relative to cell viability signal, and this ratio was then normalized relative to mock transfected cells. All transfections were performed using Lipofectamine 3000 (Invitrogen). For mRNA readout, samples were directly lysed in-well for RNA purification using the Promega Maxwell Automated RNA Extractor, followed by single-strand cDNA synthesis. The resulting cDNA was used in qRT-PCR reaction with commercially available primers designed to STAT3 and the housekeeping gene YWHAZ. mRNA levels were quantified by the standard curve method and data was analyzed by normalizing STAT3 relative to YWHAZ. This ratio was then normalized relative to mock transfected cells. All transfections were performed using Lipofectamine 3000 (Invitrogen). The data are shown in FIG. 24.

### Example 24: Evaluation of Bait-Conjugates in PC9 HiBiT Cell Lines

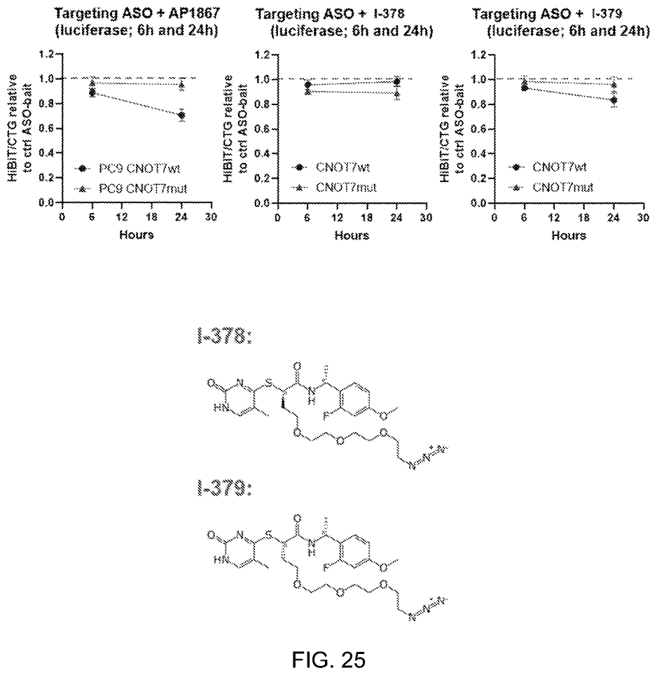

Pc9 HiBiT cell lines were created as described in Example 23. The PC9 STT3-HiBit cell lines were created but with wild type CNOT7 conjugated to FKBPV and an inactive mutant (See Example 16) of CNOT7 conjugated to FKBPV. The cells were transfected with the STAT3 targeting ASO-bait (SEQ ID NO:3; this ASO-bait is also referred to as “Pos Ctl; ASO-EL” in Example 23) conjugated to (1) a positive control bait: AP1867 (CAS NO.: 195514-23-9), a known binder of FKBPV; (2) I-378 or (3) I-379. The luciferase signal was assayed at both 6 h and 24 hrs. The results are shown in FIG. 25. The data show that CNOT7 binders, e.g., I-379, have the ability to induce degradation intracellularly in cell line expressing CNOT7 WT when compared to a cell line expressing an inactive mutant of CNOT7. The cellular data, which indicate that 1-379 has a stronger effect than 1-378, correspond to the biochemical data, see e.g., Example 17 and FIG. 18, which show that the 1-379 bait conjugated to an rSM (I-329), is more potent than 1-378 conjugated to the rSM (1-371).

1. A compound of Formula B: 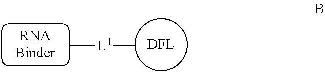
or a pharmaceutically acceptable salt thereof, wherein:
RNA Binder is a moiety that binds to a target RNA transcript;
DFL is a Decay Factor-recruiting Ligand; and
-L¹- is a bivalent linker group that covalently connects the RNA Binder to the DFL;
wherein the DFL binds to or recruits a decay factor.

2. The compound of claim 1, wherein the RNA Binder is an oligonucleotide, a polypeptide or an RNA-binding small molecule (rSM).

3. The compound of claim 1, wherein the RNA Binder is an oligonucleotide.

4. The compound of claim 1, wherein the RNA Binder is an rSM.

5. A compound of Formula A: 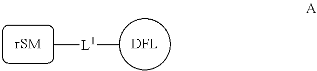
or a pharmaceutically acceptable salt thereof, wherein:
rSM is an RNA-binding small molecule that binds to a target RNA transcript;
DFL is a Decay Factor-recruiting Ligand; and
-L¹- is a bivalent linker group that covalently connects the rSM to the DFL;
wherein the DFL binds to or recruits a decay factor.

6. The compound of claim 5, wherein 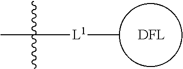
is a compound of Formula I-a: 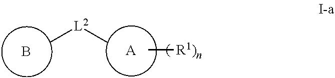
or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
Ring B 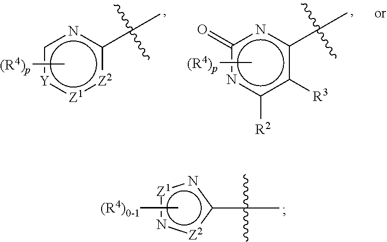
Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is 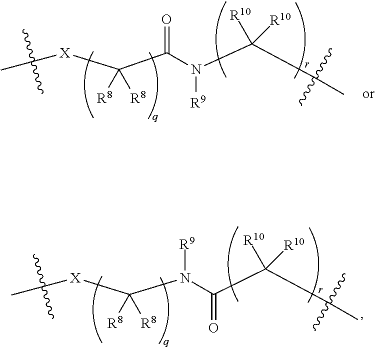
wherein —X— is covalently bound to Ring B; —X— is NR⁶, —O—, —CR⁶R⁷—, or —S—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or -L¹-;
R⁹ is —R, halogen, —CN, —NC, —C(O)OR, —OC(O)R, —OR, —N(R)₂, or —SR; or R⁸ and R⁹ taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur; each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or R⁸ and R¹⁰ or R⁹ and R¹⁰, taken together with the atoms to which they are attached, form a ring substituted with m instances of R¹; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a covalent bond or a C₁₋₈ bivalent straight or branched hydrocarbon chain wherein 1, 2, or 3 methylene units of the chain are independently and optionally replaced with —O—, —C(O)—, —C(O)O—, —OC(O)—, —N(R)—, —C(O)N(R)—, —(R)NC(O)—, —OC(O)N(R)—, —(R)NC(O)O—, —N(R)C(O)N(R)—, —S—, —SO—, —SO₂—, —SO₂N(R)—, —(R)NSO₂—, —C(S)—, —C(S)O—, —OC(S)—, —C(S)N(R)—, —(R)NC(S)—, —(R)NC(S)N(R)—, or -Cy-; wherein one and only one of R¹, R², R³, or R⁸ is -L¹- and one end of -L¹- is covalently bound to rSM;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
m is 0, 1, 2, 3, or 4;
n is 0, 1, 2, 3, or 4;
p is 0, 1, 2, or 3;
q is 0, 1, 2, 3, or 4; and
r is 0, 1, 2, 3, or 4.

7. The compound of claim 5 or 6, wherein Ring A is selected from:

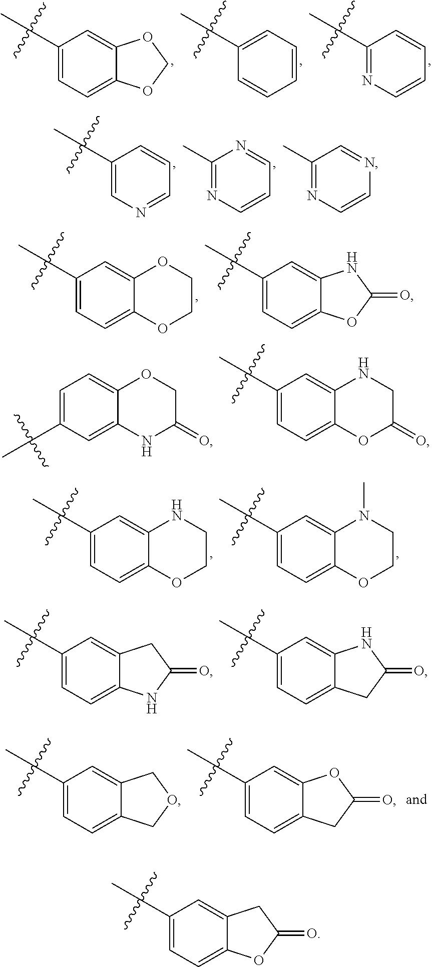

8. The compound of any one of claims 5 to 7, wherein Ring B is

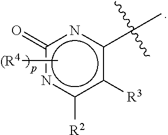

9. The compound of any one of claims 5 to 8, wherein R¹ is -L¹-.

10. The compound of any one of claims 5 to 8, wherein R² is -L¹-.

11. The compound of any one of claims 5 to 8, wherein R³ is -L¹-.

12. The compound of any one of claims 5 to 11, wherein R⁴ is selected from H, ═O, Me, Et, iPr,

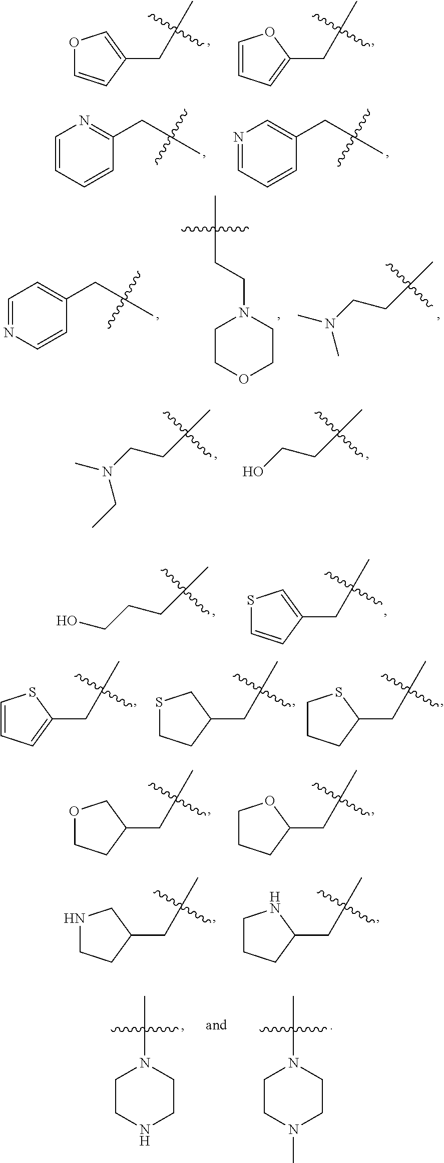

13. The compound of any one of claims 5 to 8, wherein R⁸ is -L¹-.

14. The compound of any one of claims 5 to 13, wherein -L²- is selected from

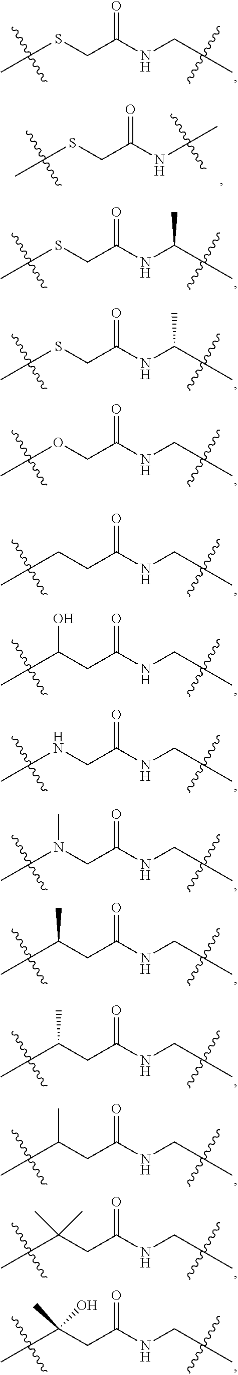

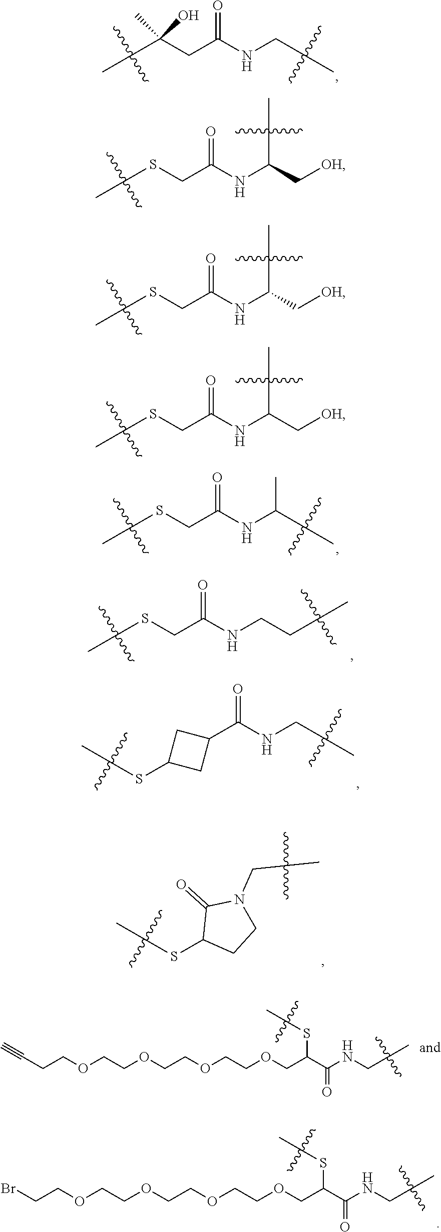

15. The compound of any one of claims 5 to 14, wherein the compound is of Formula II-a or II-b: 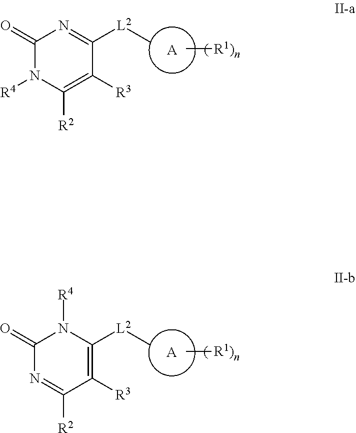
or a pharmaceutically acceptable salt thereof.

16. The compound of any one of claims 5 to 8, wherein the compound is of Formula III-a: 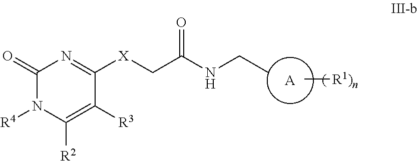
or a pharmaceutically acceptable salt thereof.

17. The compound of any one of claims 5 to 8, wherein the compound is of Formula IV-a: 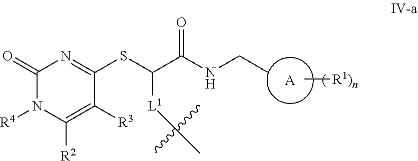
or a pharmaceutically acceptable salt thereof.

18. The compound of claim 5, wherein 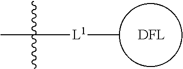
is a compound of Formula I-c: 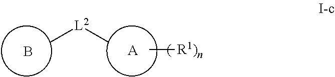
or a pharmaceutically acceptable salt thereof, wherein:
Ring A is a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic heteroaromatic ring having 1-5 heteroatoms independently selected from nitrogen, oxygen, and sulfur; an 8-12 membered bicyclic heterocyclic ring having 1-5 heteroatoms independently selected from nitrogen, oxygen, and sulfur; or an 8-12 membered tricyclic heterocyclic ring having 1-5 heteroatoms independently selected from nitrogen, oxygen, and sulfur;
Ring B is 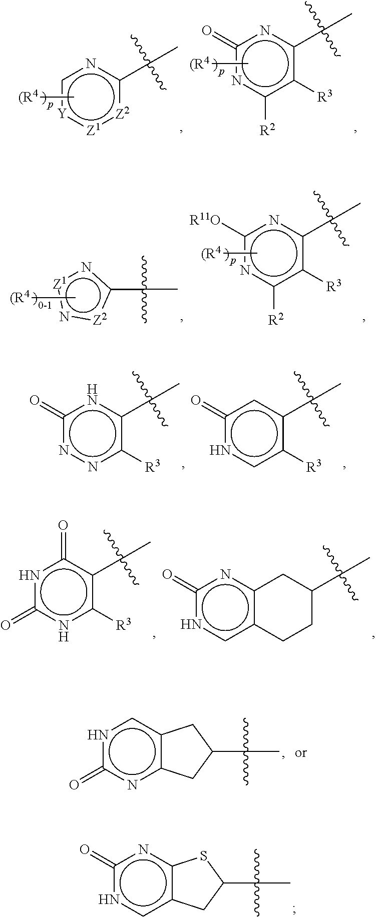
Y is N or CH;
Z¹ is N, C═O or CR²;
Z² is N, C═O, or CR³; provided that Z¹ and Z² are not both N or C═O;
each R¹ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-;
R² and R³ are each independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or R² and R³, taken together with the carbons to which they are attached, form a ring substituted with m instances of R⁵; wherein the ring is a 4-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁴ is independently —R, halogen, ═O, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R or C₁₋₈ bivalent straight or branched hydrocarbon chain wherein 1, 2, 3, or 4 methylene units of the chain are independently and optionally replaced with —O—, —C(O)—, —C(O)O—, —OC(O)—, —N(R)—, —C(O)N(R)—, —(R)NC(O)—, —OC(O)N(R)—, —S—, —SO—, or —SO₂—;
each R⁵ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R;
-L²- is 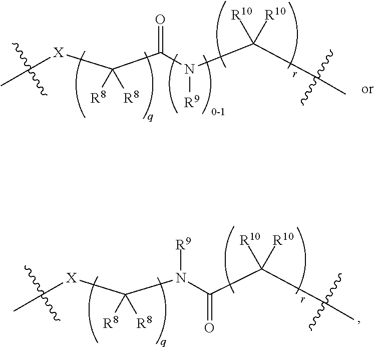
wherein —X— is covalently bound to Ring B; —X— is a bond, —NR⁶, —O—, —CR⁶R⁷—, —C(O)—, —S—, or —S(O)₂—; and one instance of —C(R⁸)₂— or —C(R¹⁰)₂— is optionally replaced by a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-3 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
each R⁶ and R⁷ is independently —R, halogen, —CN, —NC, —C(O)OR, —OC(O)R, —OR, —N(R)₂, or —SR;
each R⁸ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, —NRS(O)₂R, or -L¹-; or two R⁸, taken together with the carbon atom to which they are attached, form a 3-6 membered carbocyclic ring;
R⁹ is —R, halogen, —CN, —NC, —C(O)OR, —OC(O)R, —OR, —N(R)₂, or —SR; or R⁸ and R⁹, taken together with the atoms to which they are attached, form a ring substituted with m instances of R⁵; wherein the ring is a 5-8 membered saturated or partially unsaturated monocyclic heterocyclic ring having 1-2 heteroatoms independently selected from nitrogen, oxygen, and sulfur;
each R¹⁰ is independently —R, halogen, —CN, —NC, —C(O)OR, —OC(O)R, —C(O)N(R)₂, —N(R)C(O)R, —N(R)C(O)N(R)₂, —OC(O)N(R)₂, —N(R)C(O)OR, —OR, —N(R)₂, —NO₂, —N₃, —SR, —S(O)R, —S(O)₂R, —S(O)₂N(R)₂, or —NRS(O)₂R; or two R¹⁰, taken together with the carbon atom to which they are attached, form a 3-6 membered carbocyclic ring; or R⁹ and R¹⁰, taken together with the atoms to which they are attached, form a ring substituted with m instances of R⁵; wherein the ring is a 6-8 membered saturated or partially unsaturated monocyclic heterocyclic ring having 1-2 heteroatoms independently selected from nitrogen, oxygen, and sulfur;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, and sulfur;
-L¹- is a bivalent linker group that covalently connects the rSM to the DFL; wherein one and only one of R₁, R², R³, R⁸, or R¹¹ is -L¹- and one end of -L¹- is covalently bound to the rSM;
each -Cy- is independently a bivalent optionally substituted 3-8 membered saturated or partially unsaturated monocyclic carbocyclic ring, optionally substituted phenylene, an optionally substituted 4-8 membered saturated or partially unsaturated monocyclic heterocyclic ring having 1-3 heteroatoms independently selected from nitrogen, oxygen, and sulfur, an optionally substituted 5-6 membered monocyclic heteroaromatic ring having 1-4 heteroatoms independently selected from nitrogen, oxygen, and sulfur, an optionally substituted 8-10 membered bicyclic or bridged bicyclic saturated or partially unsaturated heterocyclic ring having 1-5 heteroatoms independently selected from nitrogen, oxygen, and sulfur, or an optionally substituted 8-10 membered bicyclic or bridged bicyclic heteroaromatic ring having 1-5 heteroatoms independently selected from nitrogen, oxygen, and sulfur;
R¹¹ is H, C₁₋₃ alkyl, or -L¹-;
m is 0, 1, 2, 3, or 4;
n is 0, 1, 2, 3, 4, or 5;
p is 0, 1, 2, or 3;
q is 0, 1, 2, 3, or 4; and
r is 0, 1, 2, 3, or 4.

19. The compound of claim 18, wherein Ring A is selected from:

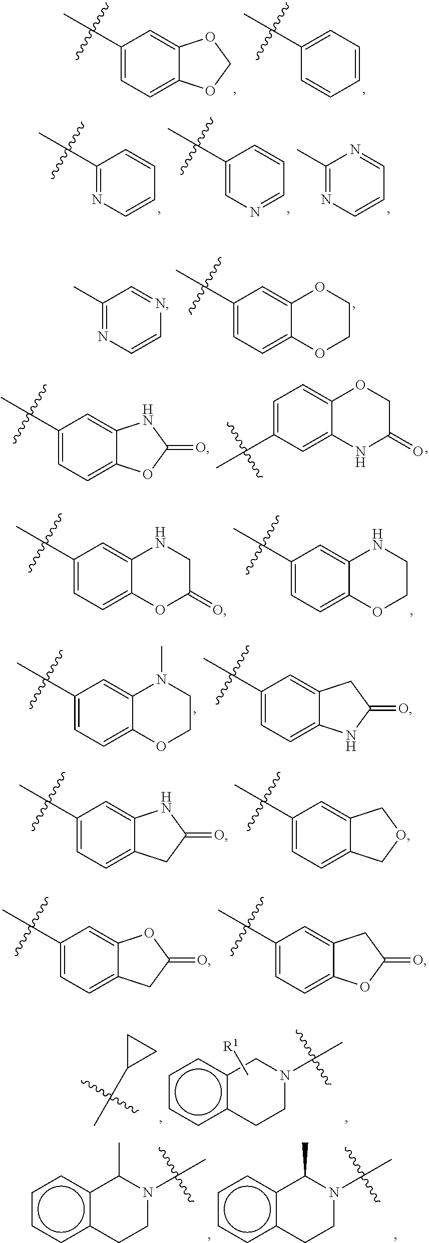

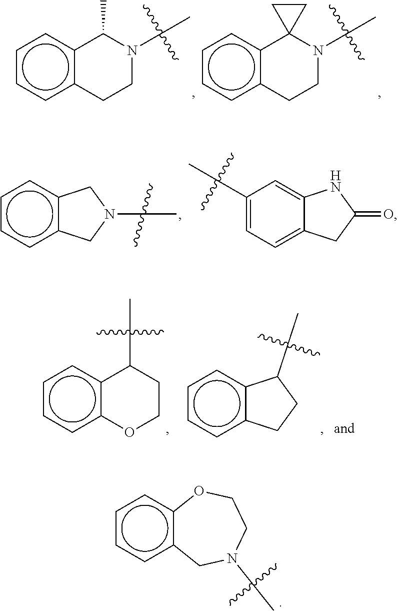

20. The compound of claim 18 or 19, wherein Ring B is

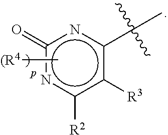

21. The compound of any one of claims 18 to 20, wherein R¹ is -L¹-.

22. The compound of any one of claims 18 to 20, wherein R² is -L¹-.

23. The compound of any one of claims 18 to 20, wherein R³ is -L¹-.

24. The compound of any one of claims 18 to 23, wherein R⁴ is selected from H, ═O, Me, Et, iPr,

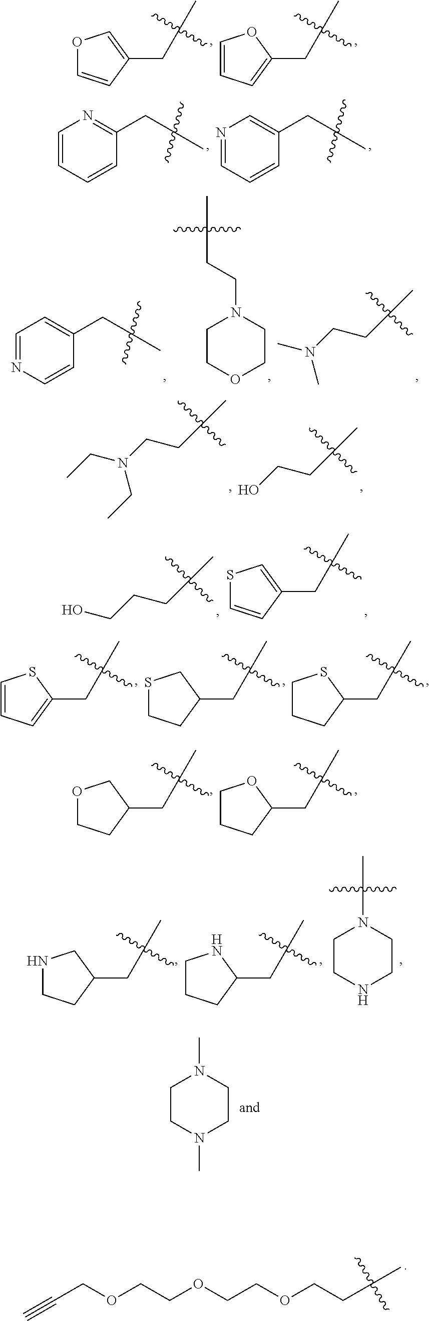

25. The compound of any one of claims 18 to 20, wherein R⁸ is -L¹-.

26. The compound of any one of claims 18 to 20, wherein R¹¹ is H, C₁₋₃ alkyl, or -L¹-.

27. The compound of any one of claims 18 to 20, wherein -L²- is selected from

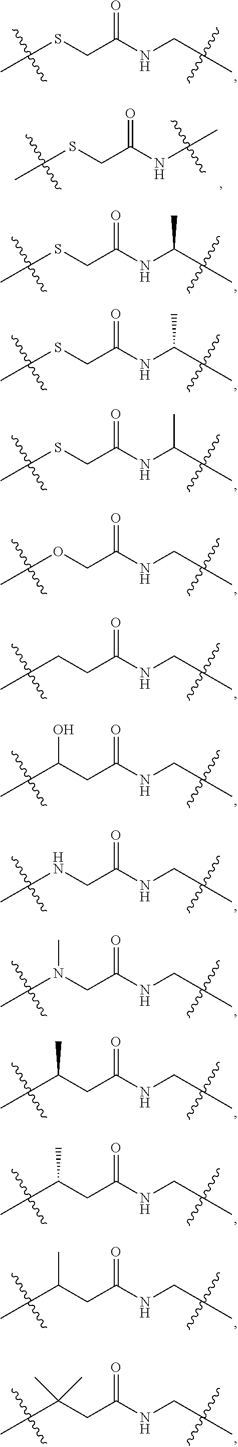

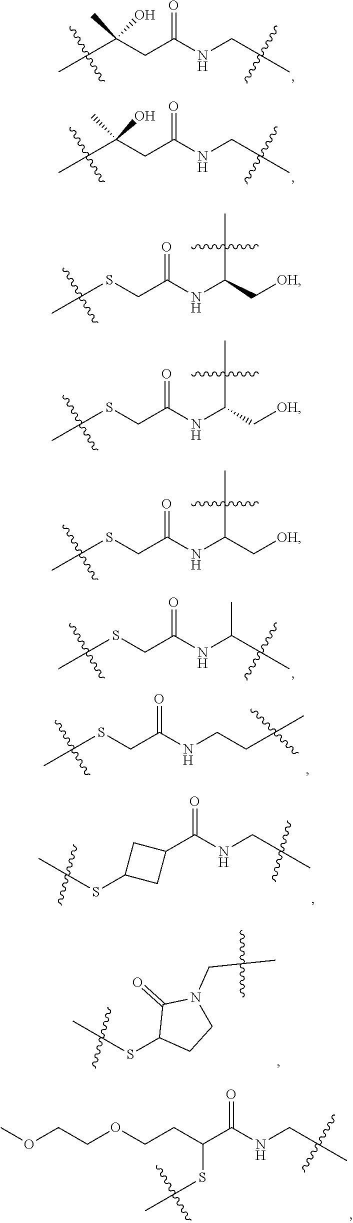

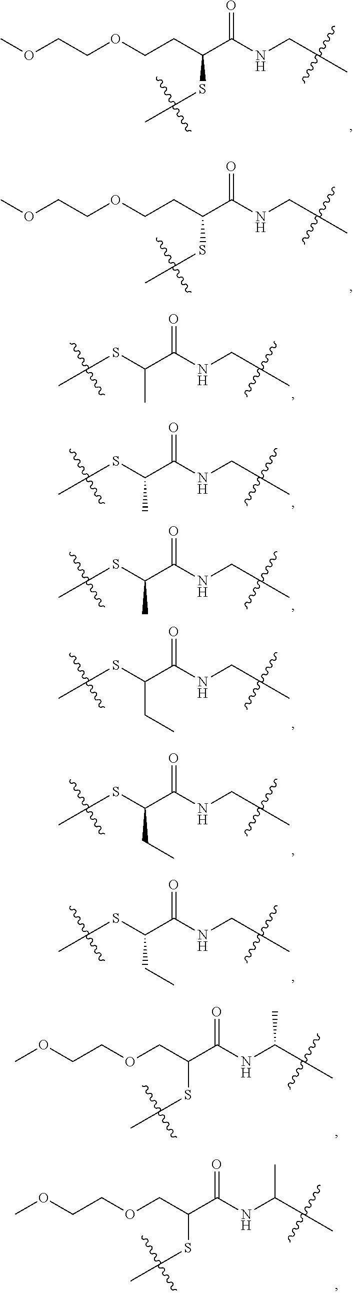

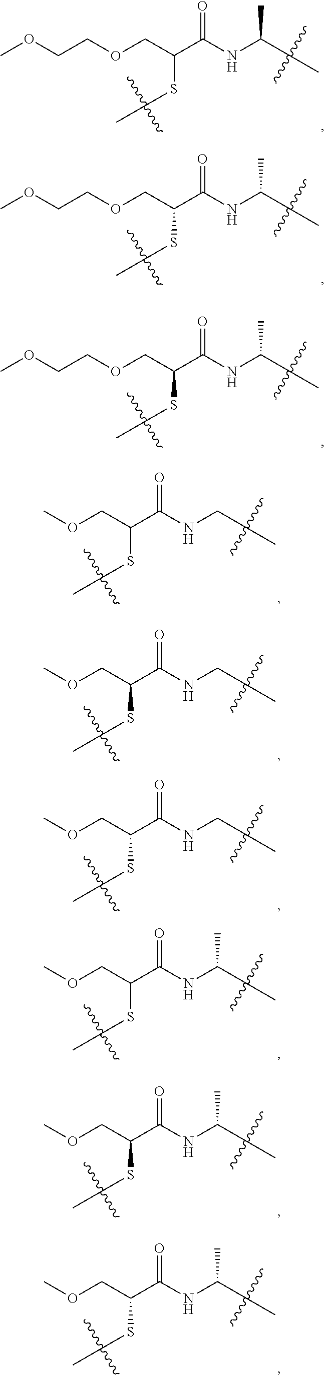

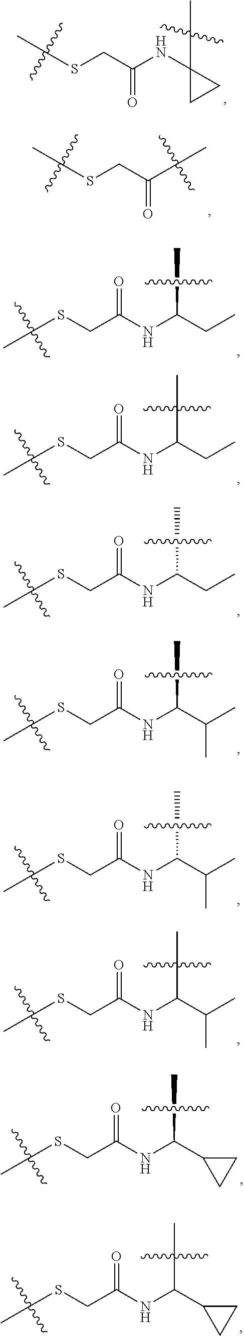

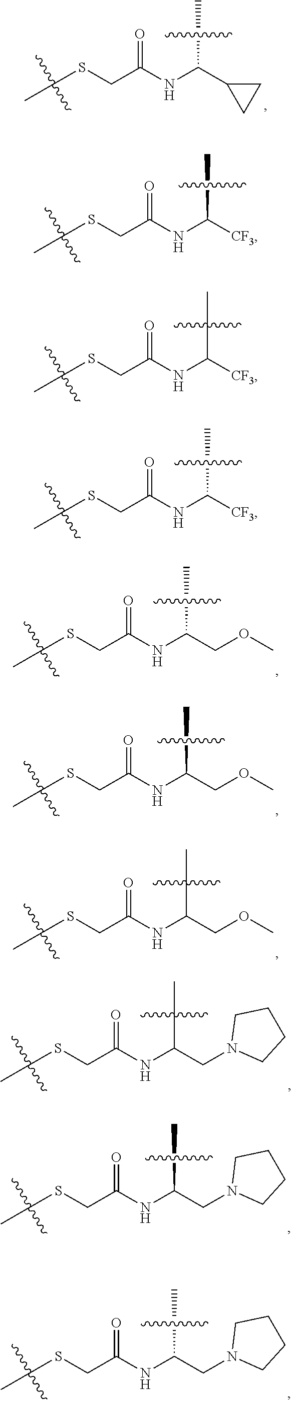

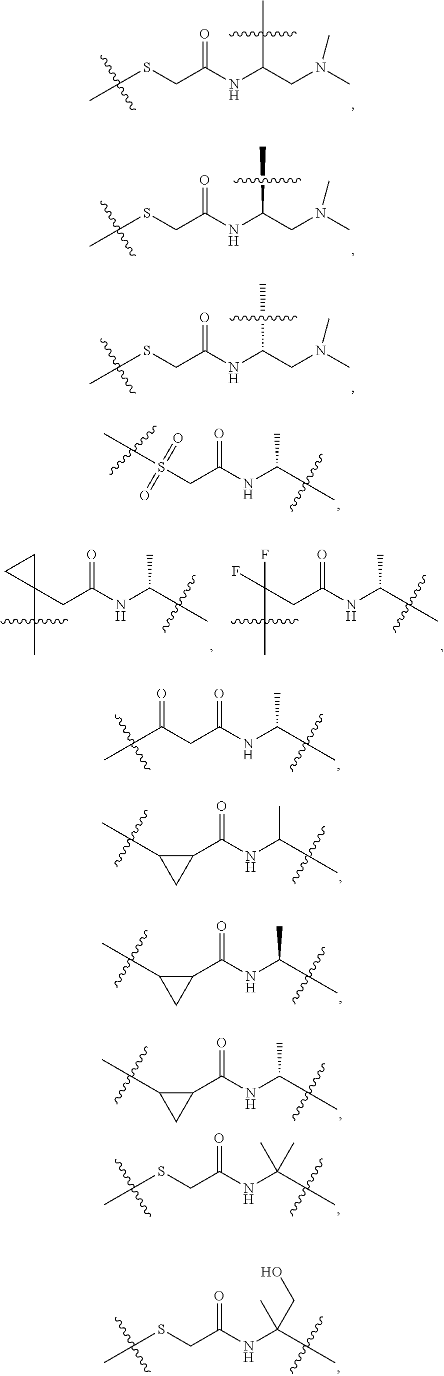

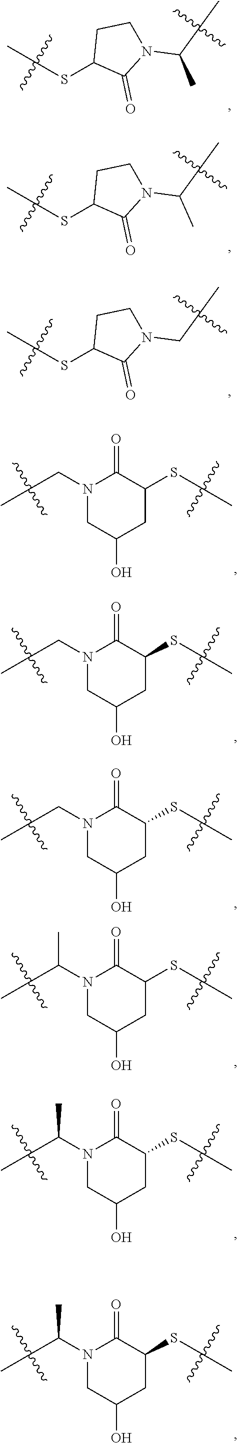

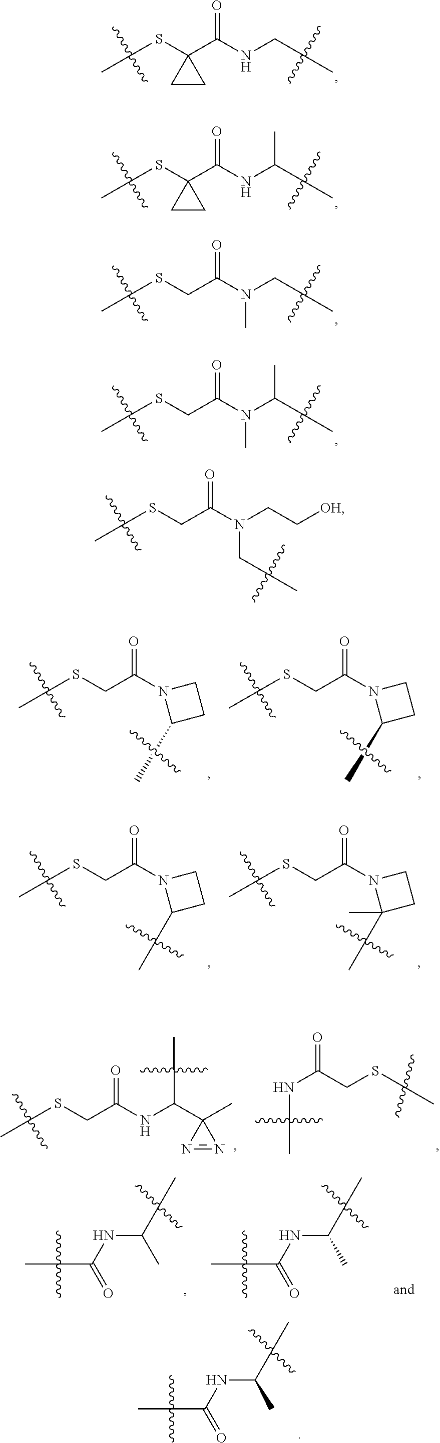

28. The compound of claim 18, wherein the compound is of Formula IX-a, IX-b, IX-c, XVI-a, XVI-b, or XVI-c: 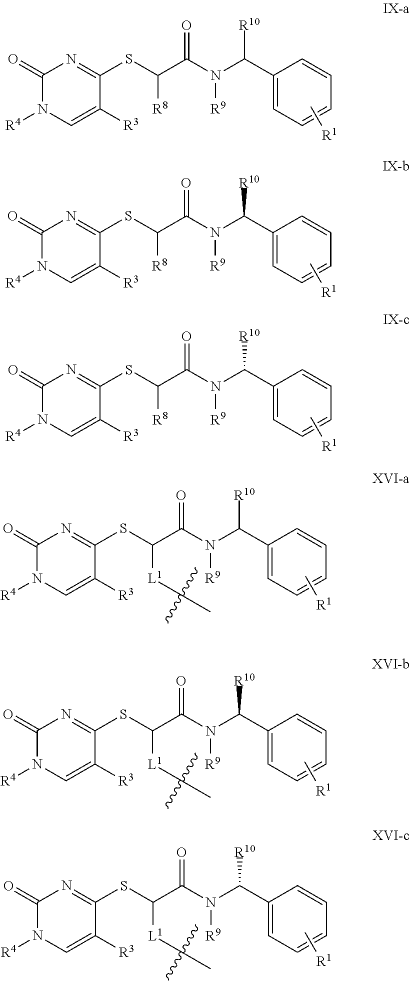
or a pharmaceutically acceptable salt thereof.

29. The compound of claim 18, wherein the compound is of Formula X-a, X-b, X-c, XVII-a, XVII-b, or XVII-c: 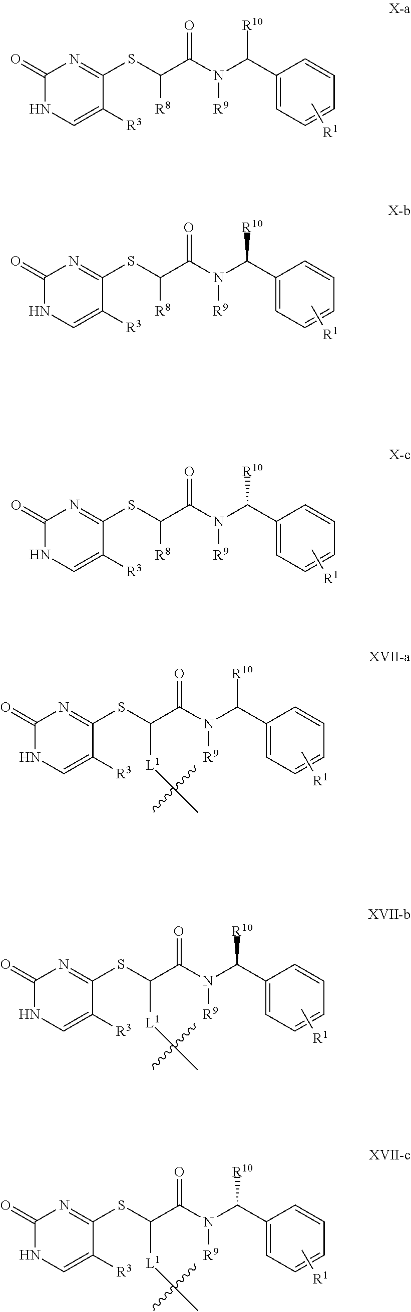
or a pharmaceutically acceptable salt thereof.

30. The compound of claim 18, wherein the compound is of Formula XI-a, XI-b, XI-c, XVIII-a, XVIII-b, or XVIII-c: 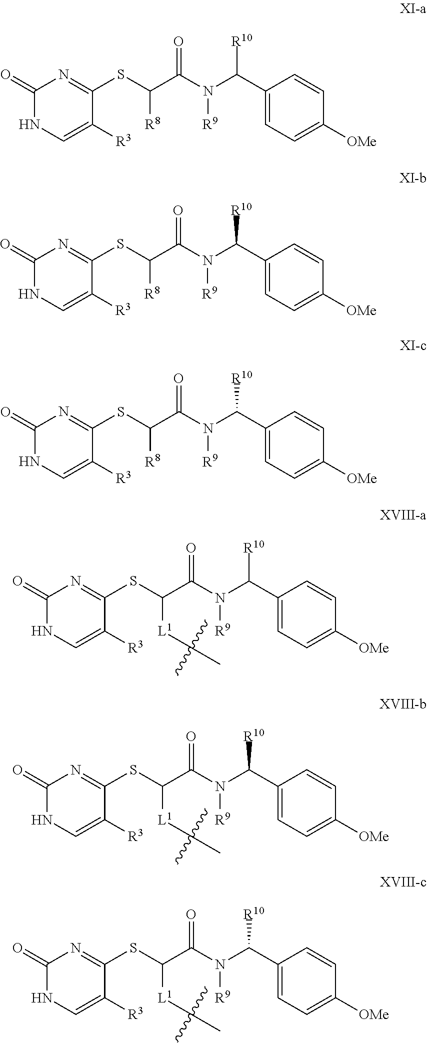
or a pharmaceutically acceptable salt thereof.

31. The compound of claim 18, wherein the compound is of Formula XII-a, XII-b, XII-c, XIX-a, XIX-b or XIX-c: 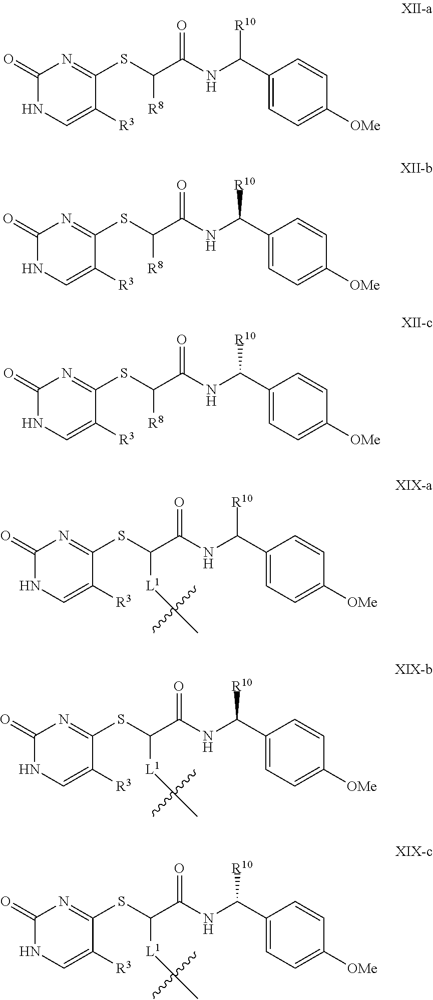
or a pharmaceutically acceptable salt thereof.

32. The compound of claim 18, wherein the compound is of Formula XIII-a, XIII-b, XIII-c, XX-a, XX-b or XX-c: 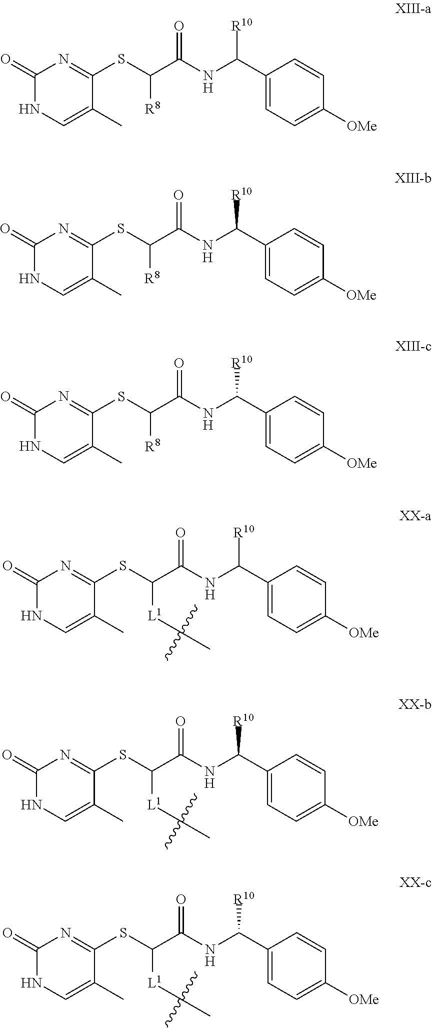
or a pharmaceutically acceptable salt thereof.

33. The compound of claim 18, wherein the compound is of Formula XIV-a, XIV-b, XIV-c, XXI-a, XXI-b or XXI-c: 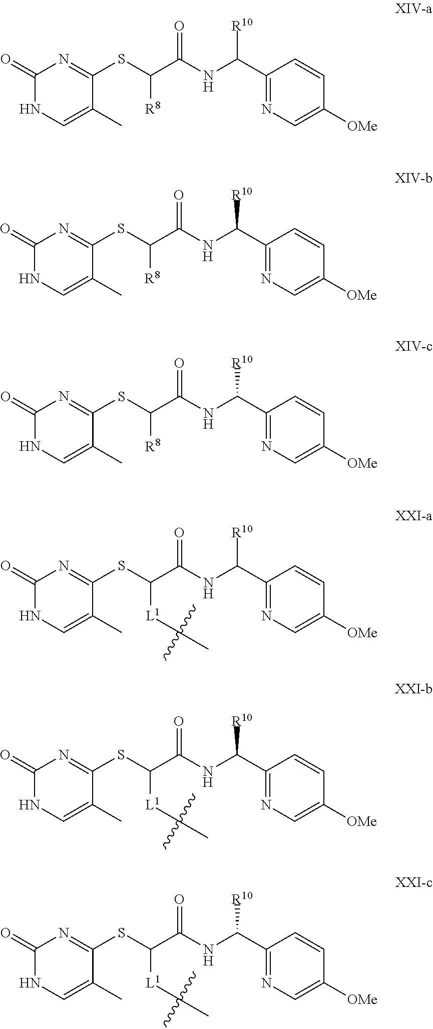
or a pharmaceutically acceptable salt thereof.

34. The compound of claim 18, wherein the compound is of Formula XV-a or XXII-a: 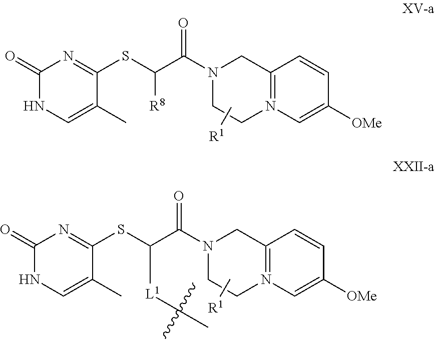
or a pharmaceutically acceptable salt thereof.

35. The compound of claim 18, wherein the compound is of Formula XXIII-a, Formula XXIII-b or Formula XXIII-c: 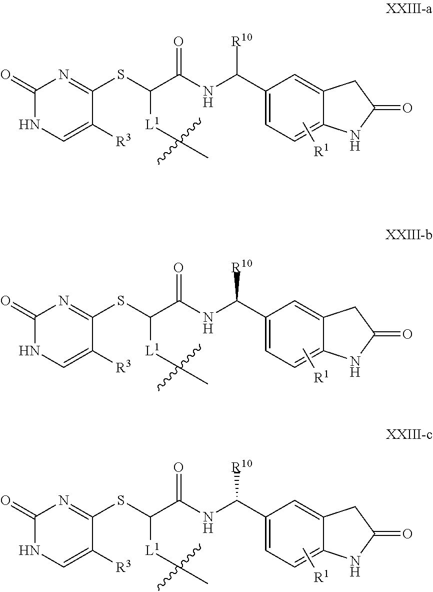
or a pharmaceutically acceptable salt thereof.

36. The compound of claim 18, wherein the compound is of Formula XXIII-d, Formula XXIII-e or Formula XXIII-f: 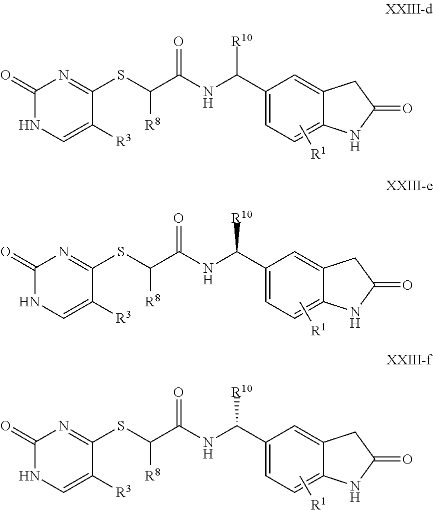
or a pharmaceutically acceptable salt thereof.

37. The compound of claim 18, wherein the compound is of Formula XXIV-a: 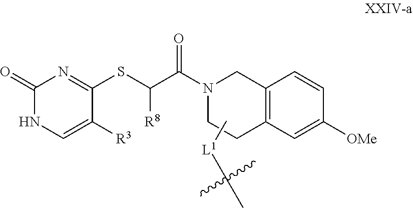
or a pharmaceutically acceptable salt thereof.

38. The compound of claim 18, wherein the compound is of Formula XXIV-b: 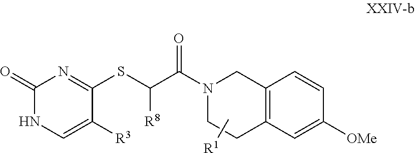
or a pharmaceutically acceptable salt thereof.

39. The compound of claim 18, wherein the compound is of Formula XXV-a: 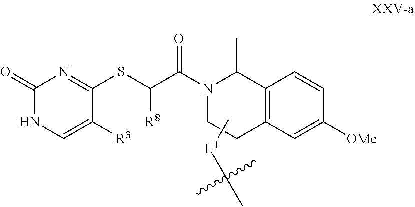
or a pharmaceutically acceptable salt thereof.

40. The compound of claim 18, wherein the compound is of Formula XXV-b: 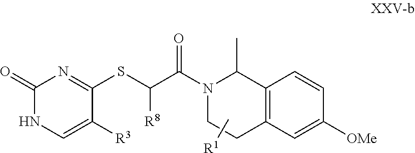
or a pharmaceutically acceptable salt thereof.

41. A compound of any of the preceding claims, wherein the decay factor is a protein that binds or interacts with RNA (an RBP) and wherein the interaction of the RBP with the RNA leads to modulation of the target RNA transcript in vivo.

42. The compound of claim 39, wherein the RBP is part of the CCR4-NOT (Carbon Catabolite Repression-Negative On TATA-less) complex.

43. The compound of claim 41 or 42, wherein the RBP is CNOT7.

44. The compound of claim 43, wherein the DFL does not bind to the active site of CNOT7.

45. The compound of claim 44, wherein the DFL binds CNOT7 without abrogating the enzymatic activity of the CNOT7 and/or the CCR4-NOT complex.

46. The compound of any one of the preceding claims, wherein the target RNA transcript is an mRNA or a precursor, isoform, unspliced isoform, splicing intermediate, fragment, or mutant thereof.

47. The compound of any one of the preceding claims, wherein the target RNA transcript is selected from one of those listed in Table C or D; or a precursor, isoform, unspliced isoform, splicing intermediate, fragment, or mutant thereof.

48. The compound of any one of the preceding claims, wherein the rSM is selected from any one of those described in paragraphs under the heading RNA-Binding Small Molecules (rSMs).

49. The compound of any one of the preceding claims, wherein the rSM is one of those shown in Table 2.

50. A pharmaceutical composition comprising the compound of any one of the preceding claims, or a pharmaceutically acceptable salt thereof, and a pharmaceutically acceptable carrier.

51. A method of modifying the amount of a protein in a cell, the method comprising administering the compound or composition of any of the preceding claims, or a pharmaceutically acceptable salt thereof, that acts on a target RNA transcript or a precursor, isoform, fragment, or mutant thereof, in an amount sufficient to modify the amount of the protein in the cell.

52. The method of claim 47, wherein modifying the amount of a protein in a cell is reducing the amount of protein in the cell.

53. A method of modulating the availability for protein translation of a target RNA transcript or a precursor, isoform, fragment, or mutant thereof, comprising contacting the target RNA transcript or a precursor, isoform, fragment, or mutant thereof with the compound or composition of any one of the preceding claims, or a pharmaceutically acceptable salt thereof, that binds to the target RNA transcript or an isoform, fragment, or mutant thereof.

54. A method of modulating the translation of a target protein or mutant thereof, comprising contacting a target RNA transcript or a precursor, isoform, fragment, or mutant thereof with the compound or composition of any one of preceding claims, or a pharmaceutically acceptable salt thereof.

55. A method of decreasing the half-life or increasing degradation of a target RNA transcript or a precursor, isoform, fragment, or mutant thereof, comprising contacting the target RNA transcript or the precursor, isoform, fragment, or mutant thereof with the compound or composition of any one of the preceding claims, or a pharmaceutically acceptable salt thereof.

56. A method of treating a disease, comprising administering to a subject in need thereof the compound or composition of any one of preceding claims, or a pharmaceutically acceptable salt thereof.

57. The method of claim 56, wherein the disease is characterized by an aberrant level of a protein in a cell.

58. The method of claim 57, wherein the disease is one of those listed in Table C or D.

59. The method of claim 58, wherein the disease is a cancer.

60. The method of any one of claims 51-59, wherein the RBP is CNOT7.
